(19) United States
(12) Patent Application Publication
(10) Pub. No.: US 2025/0059205 A1
(43) Pub. Date: Feb. 20, 2025

(54) Heterocyclic Compounds and Methods of Use

(71) Applicant: AMGEN INC., Thousand Oaks, CA (US)

(72) Inventors: Brian Alan Lanman, Woodland Hills, CA (US); Ryan Paul Wurz, Newbury Park, CA (US); Wei Zhao, Westford, MA (US); Xiaofen Li, Thousand Oaks, CA (US); Michael M. Yamano, Thousand Oaks, CA (US); Yunxiao Li, Thousand Oaks, CA (US); Ning Chen, Thousand Oaks, CA (US); Birgitte Weinreich Husemoen, Copenhagen (DK); Sebastian Leth-Petersen, Copenhagen (DK); Jose M. Medina, Thousand Oaks, CA (US); Kexue Li, Newbury Park, CA (US); Liping Pettus, Thousand Oaks, CA (US); Rene Rahimoff, Santa Monica, CA (US); Primali Vasundera Navaratne, Thousand Oaks, CA (US); Huan Rui, Los Angeles, CA (US); Christopher P. Mohr, Newbury Park, CA (US)

(73) Assignee: AMGEN INC., Thousand Oaks, CA (US)

(21) Appl. No.: 18/696,946

(22) PCT Filed: Apr. 28, 2022
(86) PCT No.: PCT/US22/26623

§ 371 Date: Mar. 28, 2024

Related U.S. Application Data
(60) Provisional application No. 63/181,625, filed on Apr. 29, 2021.
(60) Provisional application No. 63/277,309, filed on Nov. 9, 2021.

(51) Int. Cl. C07D 519/00; A61K 31/4995; A61K 31/517; C07D 487/08

(52) U.S. Cl. CPC C07D 519/00; A61K 31/4995; A61K 31/517; C07D 487/08

(57) ABSTRACT

The present disclosure provides compounds useful for the inhibition of KRAS G12D. The compounds have a general Formula I: (I) wherein the variables of Formula I are defined herein. This disclosure also provides pharmaceutical compositions comprising the compounds, uses of the compounds, and compositions for treatment of, for example, cancer.

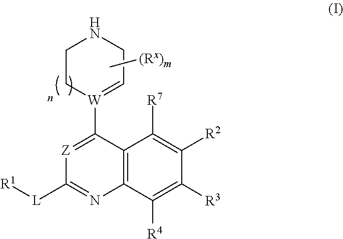

### FIELD

The present disclosure provides compounds having activity as inhibitors of G12D mutant KRAS protein. This disclosure also provides pharmaceutical compositions comprising the compounds, uses and methods of treating certain disorders, such as cancer, including but not limited to Non-Small Cell Lung Cancer (NSCLC), colorectal cancer and/or pancreatic cancer.

### BACKGROUND

From its identification as one of the first human oncogenes in 1982 (Der et al., 1982), KRAS (the Kirsten rat sarcoma viral oncogene homologue) has been the focus of extensive academic and industrial research, as a key node in the MAPK signal transduction pathway, as a transforming factor in a network of parallel effector pathways (e.g., PI3K/AKT) (Vojtek et al., 1998) and as a potential target for anti-cancer agents (Malumbres et al., 2003). Despite progress in the development of inhibitors of upstream and downstream nodes in the MAPK pathway (e.g., EGFR (Sridhar et al., 2003), BRAF (Holderfield et al., 2014) and MOK (Caunt et al., 2015), the KRAS protein has historically proven resistant to direct inhibition.

KRAS is a G-protein that couples extracellular mitogenic signaling to intracellular, pro-proliferative responses. KRAS serves as an intracellular “on/off” switch. Mitogen stimulation induces the binding of GTP to KRAS, bringing about a conformational change which enables the interaction of KRAS with downstream effector proteins, leading to cellular proliferation. Normally, pro-proliferative signaling is regulated by the action of GTPase-activating proteins (GAPs), which return KRAS to its GDP-bound, non-proliferative state. Mutations in KRAS impair the regulated cycling of KRAS between these GDP- and GTP-bound states, leading to the accumulation of the GTP-bound active state and dysregulated cellular proliferation (Simanshu et al., 2017).

Attempts to develop inhibitors of mutated KRAS proteins have historically been thwarted by the absence of druggable pockets on the surface of the protein (Cox et al., 2014). In 2013, Shokat and colleagues identified covalent inhibitors of a common (O'Bryan, 2019) oncogenic mutant of KRAS, KRAS G12C, which bound to a previously unrecognized allosteric pocket on GDP-KRAS G12C and prevented its subsequent activation (Ostream et al., 2013). This discovery brought about significant new efforts in the KRAS inhibitor research, which have recently culminated in the entry of KRAS inhibitors in human clinical trials.

While some progress has been made on KRAS G12C inhibitors, there is a continued interest and effort to develop inhibitors of KRAS, particularly inhibitors of other KRAS such as KRAS G12D. Thus, there is a need to develop new inhibitors for KRAS G12D for the treatment of disorders, such as cancer.

### SUMMARY

In one aspect, the present application is directed to compound of formula (I):

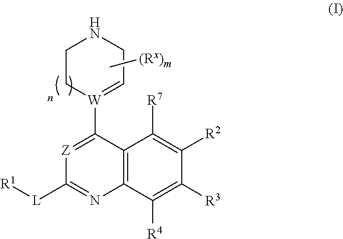

or a pharmaceutically acceptable salt of said compound, wherein;
[symbol] is a single bond or a double bond;
W is C, CH or N;
n is 0, 1, 2, or 3;
m is 0, 1, 2, 3 or 4;
each R^{x} is hydroxyl, halogen, oxo, cyano, C₁₋₄ alkyl, C₁₋₄ alkoxy, C₁₋₄ haloalkyl, —T—R^{y} or two R^{x} taken together can form a bridged ring, wherein the bridge is selected from one of the following: —C₁₋₄ alkylene, —O—C₁₋₄ alkylene, —C₁₋₄ alkylene-O—C₁₋₄ alkylene- or —C₁₋₄ alkylene-S—C₁₋₄ alkylene-;
Z is CH, CR′ or N;
R′ is halogen, cyano or C₁₋₄ alkyl;
L is a bond, C₁₋₆ alkylene, C₁₋₆ alkenylene, —O—C₁₋₆ alkylene, —S—C₁₋₆ alkylene, NR^{z}, O or S;
R¹ is hydroxyl, aryl, heteroaryl, C₃₋₈ cycloalkyl or heterocycloalkyl optionally substituted with 0-3 occurrences of R⁵;
R² is hydrogen, hydroxyl, halogen, amino, C₁₋₄ alkyl, C₁₋₄ alkoxy, C₁₋₄ haloalkyl, C₂₋₄ alkenyl, C₂₋₄ alkynyl, C₃₋₈ cycloalkyl or cyano;
R³ is aryl or heteroaryl optionally substituted with 0-4 occurrences of R⁶;
R⁴ is hydrogen, hydroxyl, halogen, C₁₋₄ alkyl, C₁₋₄ alkoxy, C₁₋₄ haloalkyl, C₂₋₄ alkenyl, C₂₋₄ alkynyl, C₃₋₈ cycloalkyl or cyano;
each R⁵ is halogen, hydroxyl, oxo, amino, C₁₋₄ alkyl or —T—R^{y};
each R⁶ is halogen, hydroxyl, amino, cyano, C₁₋₄ alkyl, C₂₋₄ alkenyl, C₂₋₄ alkynyl, C₁₋₄ haloalkyl or C₃₋₇ cycloalkyl;
R⁷ is hydrogen, halogen or C₁₋₄ alkyl;
T is C₁₋₄ alkylene, —O—, —S— or —C₁₋₄ alkylene-C(O)—;
R^{y} is halogen, hydroxyl, cyano or amino; and
R^{z} is hydrogen or C₁₋₄ alkyl;
wherein when W is N, [symbol] is a single bond, m is 2, 3, or 4 and two R^{x} are taken together to form a bridged ring, wherein the bridge is selected from one of the following: —C₁₋₄ alkylene, —C₁₋₄ alkylene-O—C₁₋₄ alkylene- or —C₁₋₄ alkylene-S—C₁₋₄ alkylene-.

In a second aspect, provided herein is a pharmaceutical composition comprising a compound of Formula I or a pharmaceutically acceptable salt of said compound and a pharmaceutically acceptable excipient.

In a third aspect, provided herein is a compound of Formula I, or a pharmaceutically acceptable salt of said compound, or the pharmaceutical composition as described herein for use in treating cancer (e.g., NSCLC, colorectal cancer or pancreatic cancer).

Reference will now be made in detail to embodiments of the present disclosure. While certain embodiments of the present disclosure will be described, it will be understood that it is not intended to limit the embodiments of the present disclosure to those described embodiments. To the contrary, reference to embodiments of the present disclosure is intended to cover alternatives, modifications, and equivalents as may be included within the spirit and scope of the embodiments of the present disclosure as defined by the appended claims.

### DETAILED DESCRIPTION

Provided herein as embodiment 1 is a compound of formula (I):

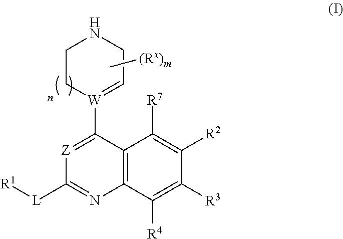

or a pharmaceutically acceptable salt of said compound, wherein;
[symbol] is a single bond or a double bond;
W is C, CH or N;
n is 0, 1, 2, or 3;
m is 0, 1, 2, 3 or 4;
each R^{x} is hydroxyl, halogen, oxo, cyano, C₁₋₄ alkyl, C₁₋₄ alkoxy, C₁₋₄ haloalkyl, —T—R^{y} or two R^{x} taken together can form a bridged ring, wherein the bridge is selected from one of the following: —C₁₋₄ alkylene, —O—C₁₋₄ alkylene, —C₁₋₄ alkylene-O—C₁₋₄ alkylene- or —C₁₋₄ alkylene-S—C₁₋₄ alkylene-;
Z is CH, CR′ or N;
R′ is halogen, cyano or C₁₋₄ alkyl;
L is a bond, C₁₋₆-alkylene, C₁₋₆-alkenylene, —O—C₁₋₆ alkylene, —S—C₁₋₆ alkylene, NR^{z}, O or S;
R¹ is hydroxyl, aryl, heteroaryl, C₃₋₈ cycloalkyl or heterocycloalkyl optionally substituted with 0-3 occurrences of R⁵;
R² is hydrogen, hydroxyl, halogen, amino, C₁₋₄ alkyl, C₁₋₄ alkoxy, C₁₋₄ haloalkyl, C₂₋₄ alkenyl, C₂₋₄ alkynyl, C₃₋₈ cycloalkyl or cyano;
R³ is aryl or heteroaryl optionally substituted with 0-4 occurrences of R⁶;
R⁴ is hydrogen, hydroxyl, halogen, C₁₋₄ alkyl, C₁₋₄ alkoxy, C₁₋₄ haloalkyl, C₂₋₄ alkenyl, C₂₋₄ alkynyl, C₃₋₈ cycloalkyl or cyano;
each R⁵ is halogen, hydroxyl, oxo, amino, C₁₋₄ alkyl or —T—R^{y};
each R⁶ is halogen, hydroxyl, amino, cyano, C₁₋₄ alkyl, C₂₋₄ alkenyl, C₂₋₄ alkynyl, C₁₋₄ haloalkyl or C₃₋₇ cycloalkyl;
R⁷ is hydrogen, halogen or C₁₋₄ alkyl;
T is C₁₋₄ alkylene, —O—, —S— or —C₁₋₄ alkylene-C(O)—;
R^{y} is halogen, hydroxyl, cyano or amino; and
R^{z} is hydrogen or C₁₋₄ alkyl;
wherein when W is N, [symbol] is a single bond, m is 2, 3, or 4 and two R^{x} are taken together to form a bridged ring, wherein the bridge is selected from one of the following: —C₁₋₄ alkylene, —C₁₋₄ alkylene-O—C₁₋₄ alkylene- or —C₁₋₄ alkylene-S—C₁₋₄ alkylene-.

Provided herein as embodiment 2 is the compound according to embodiment 1, wherein Z is CH.

Provided herein as embodiment 3 is the compound according to embodiment 1, wherein Z is CR′. Provided herein as embodiment is the compound according to embodiment 3, wherein R¹ is halogen, C₁₋₄ alkyl or cyano. Provided herein as embodiment 5 is the compound according to embodiment 4, wherein R¹ is fluoro, chloro, methyl, ethyl or cyano. (e.g., fluoro or chloro).

Provided herein as embodiment 7 is the compound according to embodiment 1, wherein Z is N.

Provided herein as embodiment 8 is the compound according to any one of embodiments 1-7, wherein W is N.

Provided herein as embodiment 9 is the compound according to any one of embodiment 8, wherein n is 1 and m is 2.

Provided herein as embodiment 10 is the compound according to embodiment 9, wherein two R^{x} taken together form a bridged ring, wherein the bridge is selected from one of the following: —C₁₋₄ alkylene, —C₁₋₄ alkylene-O—, —C₁₋₄ alkylene-O—C₁₋₄ alkylene-, —C₁₋₄ alkylene-S—C₁₋₄ alkylene- or —C₁₋₄ alkylene-S—. Provided herein as embodiment 11 is the compound according to embodiment 10, wherein two R^{x} taken together form a bridged ring, wherein the bridge is selected from one of the following: methylene, ethylene, propylene or -methylene-O-methylene-. Provided herein as embodiment 12 is the compound according to embodiment 11, wherein two R taken together form a bridged ring, wherein the bridge is —C₁₋₄ alkylene. Provided herein as embodiment 13 is the compound according to embodiment 12, wherein two R^{x} taken together form a bridged ring, wherein the bridge is methylene. Provided herein as embodiment 14 is the compound according to embodiment 12, wherein two R^{x} taken together form a bridged ring, wherein the bridge is ethylene. Provided herein as embodiment 15 is the compound according to embodiment 12, wherein two R^{x} taken together form a bridged ring, wherein the bridge is propylene.

Provided herein as embodiment 16 is the compound according to embodiment 10, wherein two R^{x} taken together form a bridged ring, wherein the bridge is —C₁₋₄ alkylene-O—C₁₋₄ alkylene (e.g., -methylene-O-methylene).

Provided herein as embodiment 17 is the compound according to embodiment 8, wherein n is 1 and m is 3.

Provided herein as embodiment 18 is the compound according to embodiment 17, wherein one R^{x} is halogen (e.g., fluorine), C₁₋₄ alkyl (e.g., methyl or ethyl), cyano, oxo or —T—R^{y} (e.g., —CH₂OH or —CH₂CN) and the other two R^{x} are taken together to form a bridged ring, wherein the bridge is —C₁₋₄ alkylene (e.g., methylene or ethylene) or —C₁₋₄ alkylene-O—C₁₋₄ alkylene (e.g., -methylene-O-methylene-)

Provided herein as embodiment 19 is the compound according to embodiment 18, wherein one R^{x} is oxo and the other two R^{x} are taken together to form a bridged ring, wherein the bridge is —C₁₋₄ alkylene (e.g., ethylene). Provided herein as embodiment 20 is the compound according to embodiment 18, wherein one R^{x} is halogen (e.g., fluorine) and the other two R^{x} are taken together to form a bridged ring, wherein the bridge is —C₁₋₄ alkylene (e.g., ethylene).

Provided herein as embodiment 21 is the compound according to embodiment 8, wherein n is 1 and m is 4.

Provided herein as embodiment 22 is the compound according embodiment 21, wherein two R^{x} are each independently C₁₋₄ alkyl (e.g., methyl) and the other two R^{x} are taken together to form a bridged ring, wherein the bridge is C₁₋₄ alkylene (e.g., ethylene) or —C₁₋₄ alkylene-O—C₁₋₄ alkylene- (e.g., -methylene-O-methylene-).

Provided herein as embodiment 23 is the compound according to embodiment 8, wherein n is 2 and m is 2.

Provided herein as embodiment 24 is the compound according to embodiment 23, wherein two R^{x} taken together form a bridged ring, wherein the bridge is —C₁₋₄ alkylene (e.g., ethylene). Provided herein as embodiment 25 is the compound according to embodiment 24, wherein two R^{x} taken together form a bridged ring, wherein the bridge is ethylene.

Provided herein as embodiment 26 is the compound of any one of embodiments 1-7, wherein W is C. Provided herein as embodiment 27 is the compound according to embodiment 26, wherein [symbol] is a double bond.

Provided herein as embodiment 2 according to embodiment 26 or 27, wherein n is 1 and m is 2.

Provided herein as embodiment 29 is the compound according to embodiment 28, wherein two R^{x} are taken together to form a bridged ring, wherein the bridge is C₁₋₄ alkylene (e.g., ethylene).

Provided herein as embodiment 30 is the compound according to any one of embodiments 1-29, wherein

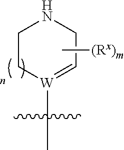

is

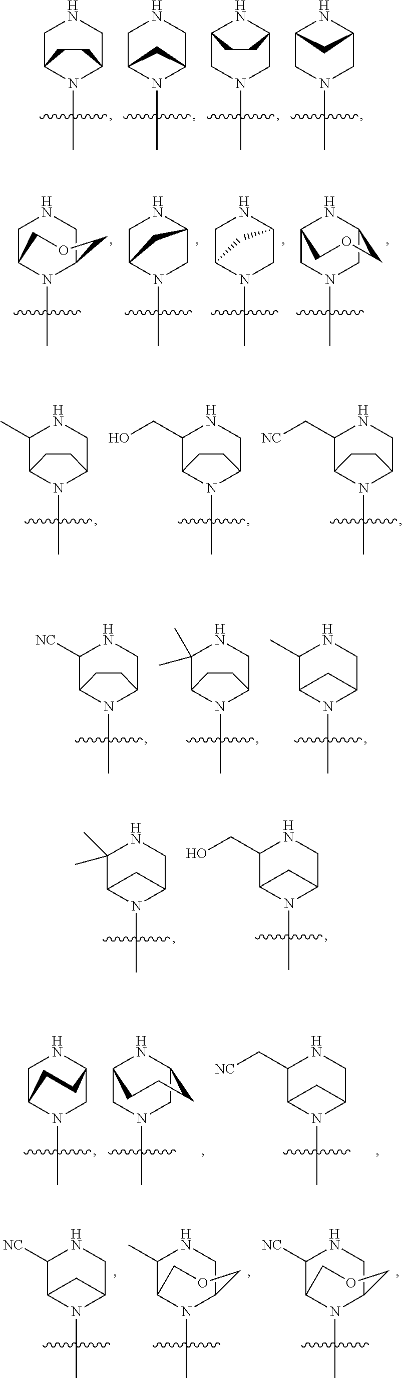

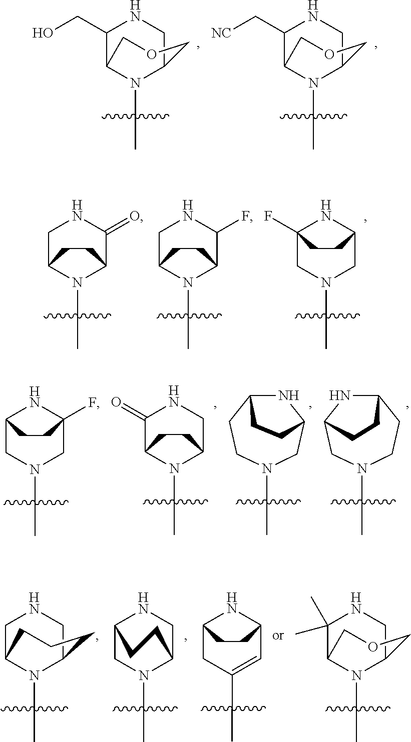

Provided herein as embodiment 31 is the compound according to embodiment 30, wherein

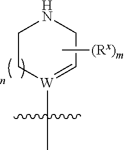

is

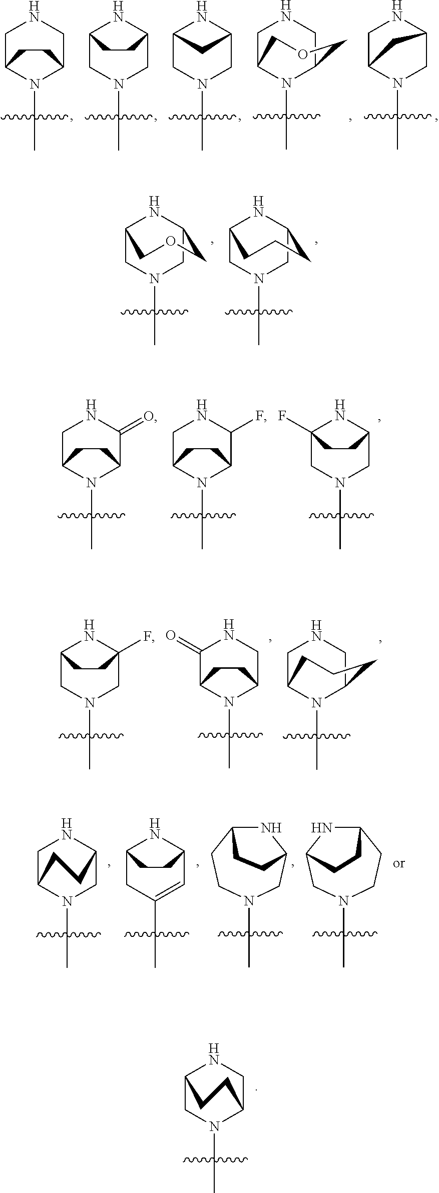

Provided herein as embodiment 32 is the compound according to embodiment 31, wherein

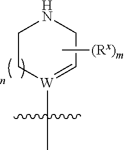

is

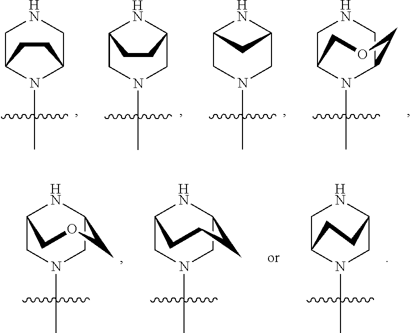

Provided herein as embodiment 33 is the compound according to embodiment 30, wherein

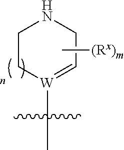

is

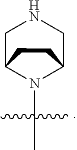

Provided herein as embodiment 34 is the compound according to embodiment 30, wherein

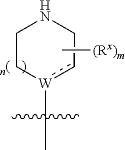

is,

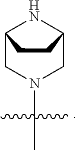

Provided herein as embodiment 35 is the compound according to embodiment 30, wherein

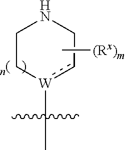

is

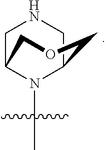

Provided herein as embodiment 36 is the compound according to embodiment 30 wherein

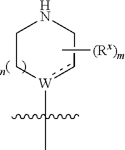

is

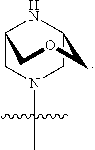

Provided herein as embodiment 37 is the compound according to embodiment 30, wherein

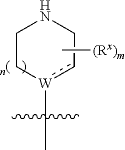

is

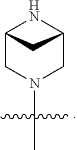 [SMILES: *N1C[C@H]2C[C@@H](C1)N2]

Provided herein as embodiment 38 is the compound according to embodiment 30, wherein

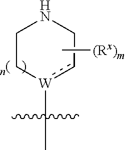

is

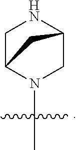 [SMILES: *N1C[C@@H]2C[C@H]1CN2]

Provided herein as embodiment 39 is the compound according to embodiment 30 wherein

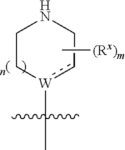

is

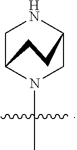

Provided herein an embodiment 40 is the compound according to embodiment 30, wherein

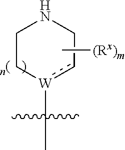

is

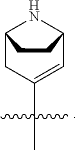

Provided herein an embodiment 41 is the compound according to embodiment 30, wherein

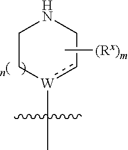

is

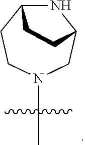

Provided herein an embodiment 42 is the compound according to embodiment 30, wherein

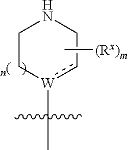

is

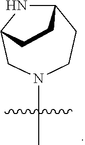

Provided herein an embodiment 43 is the compound according to embodiment 30, wherein

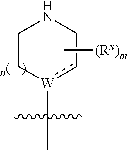

is

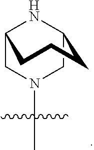

Provided herein an embodiment 44 is the compound according to embodiment 30, wherein

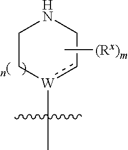

is

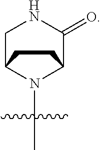

Provided herein an embodiment 45 is the compound according to embodiment 30, wherein

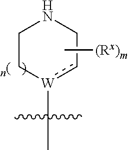

is

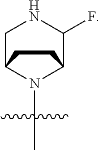

Provided herein an embodiment 46 is the compound according to embodiment 30, wherein

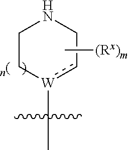

is

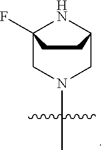

Provided herein an embodiment 47 is the compound according to embodiment 30, wherein

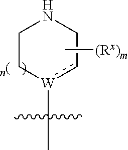

is

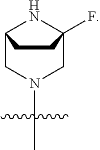

Provided herein an embodiment 48 is the compound according to embodiment 30, wherein

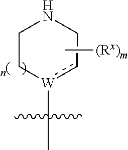

is

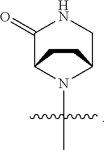

Provided herein an embodiment 49 is the compound according to embodiment 30, wherein

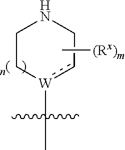

is

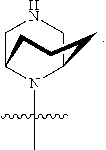

Provided herein an embodiment 50 is the compound according to embodiment 30, wherein

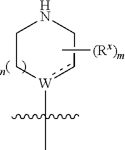

is

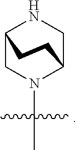

Provided herein as embodiment 51 is the compound according to any one of embodiments 1-50, wherein L is —O—C₁₋₆ alkylene (e.g., —O-methylene-, —O-ethylene-, —O-isopentanylene, —O-n-propyl-, —O—(2-methylpropyl)-, —O—(2-methylbutyl)-, —O—(2-ethylbutyl)-, —O—1,2-dimethylpropyl- or —O—(3-methylbutyl)-). Provided herein as embodiment 52 is the compound according to embodiment 51, wherein L is —O-methylene or —O-ethylene. Provided herein as embodiment 53 is the compound according to embodiment 52, wherein L is —O-methylene. Provided herein as embodiment 54 is the compound according to embodiment 52, wherein L is —O-ethylene.

Provided herein as embodiment 55 is the compound according to any one of embodiments 1-54, wherein R¹ is heterocycloalkyl optionally substituted with 0-3 occurrences of R⁵. Provided herein as embodiment 56 is the compound according to embodiment 55, wherein R¹ is 7-(hexahydro-1H-pyrrolizine), 2-pyrrolidine or 2-tetrahydrofuranyl substituted with 0-3 occurrences of R⁵.

Provided herein as embodiment 57 is the compound according to embodiment 56, wherein R¹ is 7-(hexahydro-1H-pyrrolizine) substituted with 0-3 occurrences of R⁵. Provided herein as embodiment 58 is the compound according to embodiment 57, wherein R¹ is 7-(hexahydro-1H-pyrrolizine) substituted with 1 occurrence of R⁵. Provided herein as embodiment 59 is the compound according to embodiment 58, wherein R⁵ is halogen (e.g., fluorine).

Provided herein as embodiment 60 is the compound according to embodiment 57, wherein R¹ is 7-(hexahydro-1H-pyrrolizine) substituted with 2 occurrences of R⁵. Provided herein as embodiment 61 is the compound according to embodiment 60, wherein both R⁵ are halogen (e.g., fluorine).

Provided herein as embodiment 62 is the compound according to embodiment 56, wherein R¹ is 2-pyrrolidine substituted with 0-3 occurrences of R⁵. Provided herein as embodiment 63 is the compound according to embodiment 62, wherein R¹ is 2-pyrrolidine substituted with 1 occurrence of R⁵. Provided herein as embodiment 64 is the compound according to embodiment 63, wherein R⁵ is halogen, C₁₋₄ alkyl or oxo. Provided herein as embodiment 65 is the compound according to embodiment 64, wherein R⁵ is fluorine, chlorine, methyl or oxo.

Provided herein as embodiment 66 is the compound according to embodiment 62, wherein R¹ is 2-pyrrolidine substituted with 2 occurrences of R⁵. Provided herein as embodiment 67 is the compound according to embodiment 66, wherein each R⁵ is halogen, C₁₋₄ alkyl or oxo. Provided herein as embodiment 68 is the compound according to embodiment 67, wherein each R⁵ is fluorine, methyl or oxo.

Provided herein as embodiment 69 is the compound according to embodiment 56, wherein R¹ is 2-tetrahydrofuranyl substituted with 0-3 occurrences of R⁵. Provided herein as embodiment 70 is the compound according to embodiment 69, wherein R¹ is 2-tetrahydrofuranyl substituted with 2 occurrences of R⁵. Provided herein as embodiment 71 is the compound according to embodiment 70, wherein one R⁵ is oxo and the other R⁵ is C₁₋₄ alkyl (e.g., methyl).

Provided herein as embodiment 72 is the compound according to any one of embodiments 1-54, wherein R¹ is heteroaryl optionally substituted with 0-3 occurrences of R⁵. Provided herein as embodiment 73 is the compound according to embodiment 72, wherein R¹ is 2-imidazolyl optionally substituted with 0-3 occurrences of R⁵. Provided herein as embodiment 74 is the compound according to embodiment 73, wherein R¹ is 2-imidazolyl optionally substituted with 1 occurrence of R⁵. Provided herein as embodiment 75 is the compound according to embodiment 74, wherein R⁵ is C₁₋₄ alkyl (e.g., methyl).

Provided herein as embodiment 76 is the compound according to any one of embodiments 1-54, wherein R¹ is C₃₋₈ cycloalkyl substituted with 0-3 occurrences of R⁵. Provided herein as embodiment 77 is the compound according to embodiment 76, wherein R¹ is cyclopropyl, cyclobutyl or cyclopentyl substituted with 0-3 occurrences of R⁵.

Provided herein as embodiment 78 is the compound according to embodiment 77, wherein R¹ is cyclopropyl substituted with 0-3 occurrences of R⁵. Provided herein as embodiment 79 is the compound according to embodiment 78, wherein R¹ is cyclopropyl substituted with one occurrence of R⁵. Provided herein as embodiment 80 is the compound according to embodiment 79, wherein R⁵ is halogen (e.g., fluorine or chlorine) or hydroxyl.

Provided herein as embodiment 81 is the compound according to embodiment 77, wherein R¹ is cyclobutyl substituted with 0-3 occurrences of R⁵. Provided herein as embodiment 82 is the compound according to embodiment 81, wherein R¹ is cyclobutyl substituted with one occurrence of R⁵, wherein R⁵ is hydroxyl.

Provided herein as embodiment 83 is the compound according to embodiment 77, wherein R¹ is cyclopentyl substituted with 0-3 occurrences of R⁵. Provided herein as embodiment 84 is the compound according to embodiment 83, wherein R¹ is cyclopentyl substituted with one occurrence of R⁵, wherein R⁵ is hydroxyl.

Provided herein as embodiment 85 is the compound according to embodiment 51, wherein L is —O-isopentanylene (i.e., —O—2,2-dimethylethylene). Provided herein as embodiment 86 is the compound according to embodiment 85, wherein R¹ is hydroxyl.

Provided herein as embodiment 87 is the compound according to embodiment 51, wherein L is —O-n-propyl. Provided herein as embodiment 88 is the compound according to embodiment 87, wherein R¹ is hydroxyl.

Provided herein as embodiment 89 is the compound according to embodiment 51, wherein L is —O-(2-methylpropyl). Provided herein as embodiment 90 is the compound according to embodiment 89, wherein R¹ is hydroxyl.

Provided herein as embodiment 91 is the compound according to embodiment 51, wherein L is —O-(2-methylbutyl). Provided herein as embodiment 92 is the compound according to embodiment 91, wherein R¹ is hydroxyl.

Provided herein as embodiment 93 is the compound according to embodiment 51, wherein L is —O-(2-ethylbutyl). Provided herein as embodiment 94 is the compound according to embodiment 93, wherein R¹ is hydroxyl.

Provided herein as embodiment 95 is the compound according to embodiment 51, wherein L is —O—1,2-dimethylpropyl. Provided herein as embodiment 96 is the compound according to embodiment 95, wherein R¹ is hydroxyl.

Provided herein as embodiment 97 is the compound according to embodiment 51, where L is —O-(3-methylbutyl). Provided herein as embodiment 98 is the compound according to embodiment 97, wherein R¹ is hydroxyl.

Provided herein as embodiment 99 is compound according to embodiment 51, wherein L is C₁₋₆ alkenylene (e.g., 3-methyl-buten-1-yl). Provided herein as embodiment 100 is the compound according to embodiment 99, wherein R¹ is hydroxyl.

Provided herein as embodiment 101 is the compound according to any one of embodiments 1-100 wherein —L—R¹ is

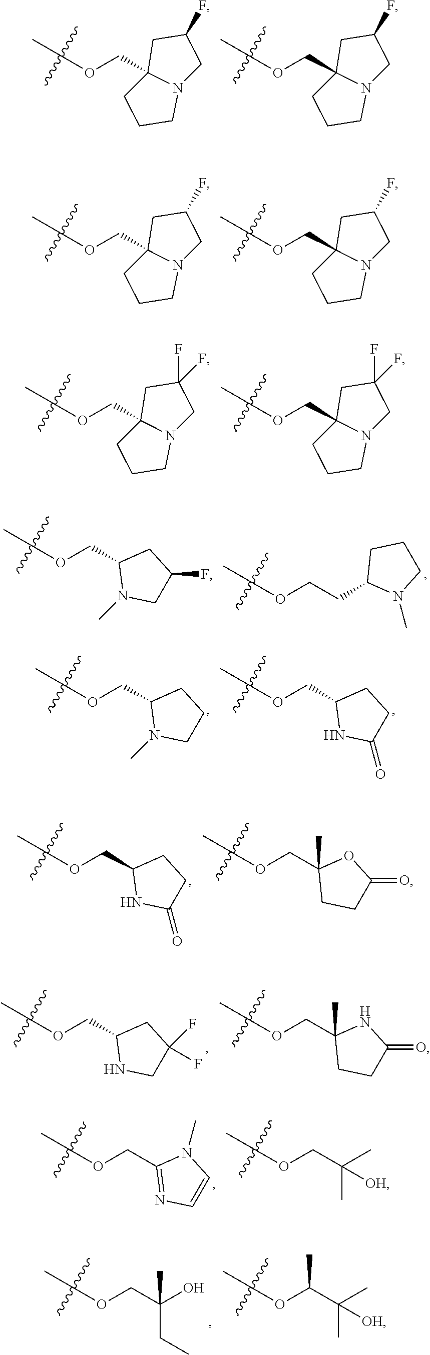

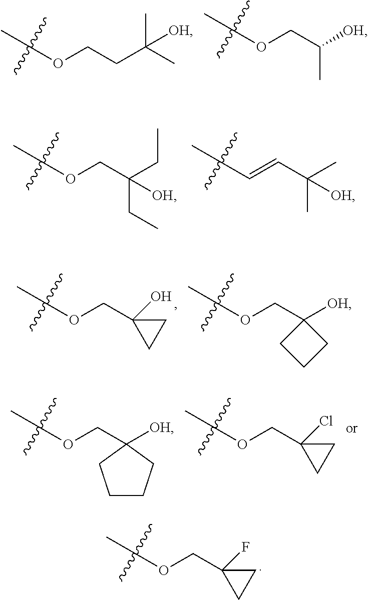

Provided herein as embodiment 102 is the compound according to embodiment 101, wherein —L—R¹ is

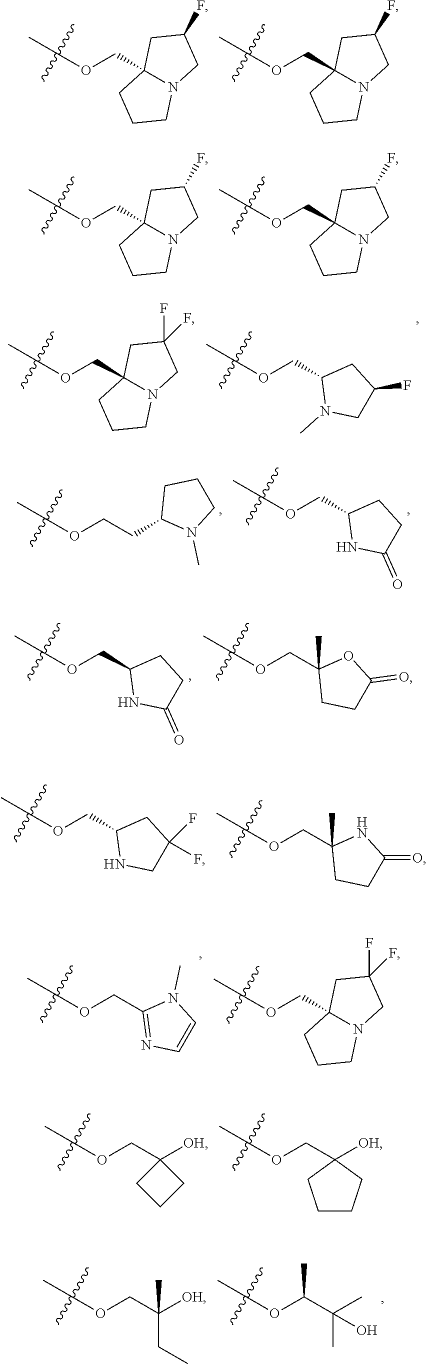

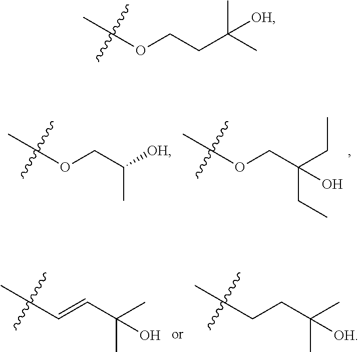

Provided herein as embodiment 103 is the compound according to embodiment 102, wherein —L—R¹ is

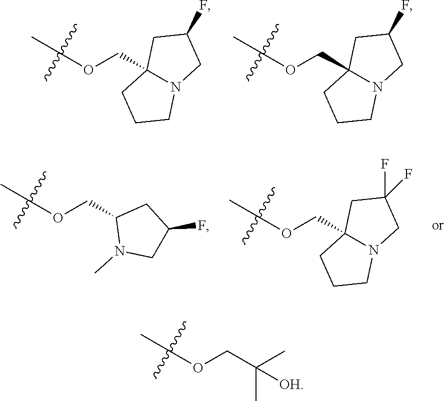

Provided herein as embodiment 104 is the compound according to embodiment 101, wherein —L—R¹ is

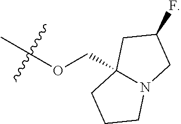 [SMILES: *OC[C@@]12CCCN1C[C@H](F)C2]

Provided herein as embodiment 105 is the compound according to embodiment 101, wherein —L—R¹ is

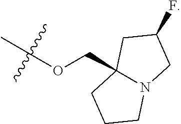 [SMILES: *OC[C@]12CCCN1C[C@H](F)C2]

Provided herein as embodiment 106 is the compound according to embodiment 101, wherein —L—R¹ is

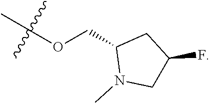 [SMILES: *OC[C@@H]1C[C@@H](F)CN1C]

Provided herein as embodiment 107 is the compound according to embodiment 101, wherein —L—R¹ is

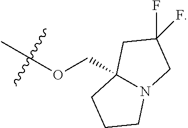 [SMILES: *OC[C@@]12CCCN1CC(F)(F)C2]

Provided herein as embodiment 108 is the compound according to embodiment 101, wherein —L—R¹ is

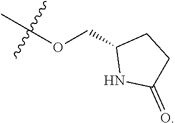 [SMILES: *OC[C@@H]1CCC(=O)N1]

Provided herein as embodiment 109 is the compound according to embodiment 101, wherein —L—R¹ is

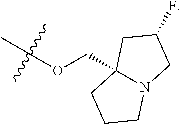 [SMILES: *OC[C@@]12CCCN1C[C@@H](F)C2]

Provided herein as embodiment 110 is the compound according to embodiment 101, wherein —L—R¹ is

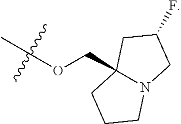 [SMILES: *OC[C@]12CCCN1C[C@@H](F)C2]

Provided herein as embodiment 111 is the compound according to embodiment 101, wherein —L—R¹ is

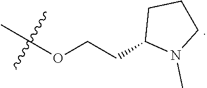 [SMILES: *OCC[C@@H]1CCCN1C]

Provided herein as embodiment 112 is the compound according to embodiment 101, wherein —L—R¹ is

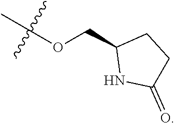 [SMILES: *OC[C@H]1CCC(=O)N1]

Provided herein as embodiment 113 is the compound according to embodiment 101, wherein —L—R¹ is

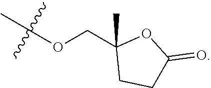 [SMILES: *OC[C@@]1(C)CCC(=O)O1]

Provided herein as embodiment 114 is the compound according to embodiment 101, wherein —L—R¹ is

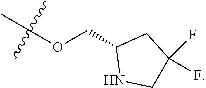 [SMILES: *OC[C@@H]1CC(F)(F)CN1]

Provided herein as embodiment 115 is the compound according to embodiment 101, wherein —L—R¹ is

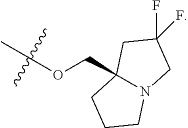 [SMILES: *OC[C@]12CCCN1CC(F)(F)C2]

Provided herein as embodiment 116 is the compound according to embodiment 101, wherein —L—R¹ is

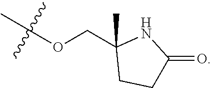 [SMILES: *OC[C@@]1(C)CCC(=O)N1]

Provided herein as embodiment 117 is the compound according to embodiment 101, wherein —L—R¹ is

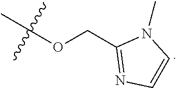 [SMILES: *OCc1nccn1C ; "."]

Provided herein as embodiment 118 is the compound according to embodiment 101, wherein —L—R¹ is

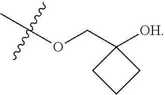 [SMILES: *OCC1(O)CCC1]

Provided herein as embodiment 119 is the compound according to embodiment 101, wherein —L—R¹ is

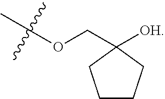 [SMILES: *OCC1(O)CCCC1]

Provided herein as embodiment 120 is the compound according to embodiment 101, wherein —L—R¹ is

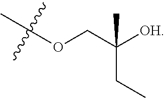 [SMILES: *OC[C@](C)(O)CC]

Provided herein as embodiment 121 is the compound according to embodiment 101, wherein —L—R¹ is

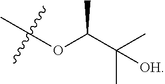 [SMILES: *O[C@@H](C)C(C)(C)O]

Provided herein as embodiment 122 is the compound according to embodiment 101, wherein —L—R¹ is

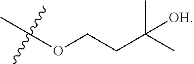 [SMILES: *OCCC(C)(C)O]

Provided herein as embodiment 123 is the compound according to embodiment 101, wherein —L—R¹ is

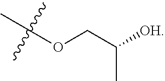 [SMILES: *OC[C@@H](C)O]

Provided herein as embodiment 124 is the compound according to embodiment 101, wherein —L—R¹ is

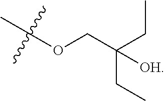 [SMILES: *OCC(O)(CC)CC]

Provided herein as embodiment 125 is the compound according to embodiment 101, wherein —L—R¹ is

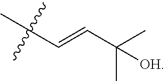 [SMILES: */C=C/C(C)(C)O]

Provided herein as embodiment 126 is the compound according to embodiment 101, wherein —L—R¹ is

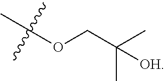 [SMILES: *OCC(C)(C)O]

Provided herein as embodiment 127 is the compound according to any one of embodiments 1-126, wherein R³ is aryl (e.g., phenyl or naphthyl) substituted with 0-3 occurrences of R⁶. Provided herein as embodiment 128 is the compound according to embodiment 127, wherein R³ is aryl substituted with one occurrence of R⁶.

Provided herein as embodiment 129 is the compound according to embodiment 128, wherein R³ is naphthyl substituted with 1 occurrence of R⁶. Provided herein as embodiment 130 is the compound according to embodiment 129, wherein R⁶ is halogen, amino, cyano, C₁₋₄ alkyl (e.g., methyl or ethyl), C₁₋₄ haloalkyl (e.g., trifluoromethyl or difluoromethyl), hydroxyl, C₂₋₄ alkenyl (e.g., 2-ethenyl) or C₂₋₄ alkynyl (e.g., 2-ethynyl). Provided herein as embodiment 131 is the compound according to embodiment 130, wherein R⁶ is fluorine, chlorine, amino, cyano, methyl, ethyl, trifluoromethyl, difluoromethyl, hydroxy, 2-ethenyl or 2-ethynyl. Provided herein as embodiment 132 is the compound according to embodiment 131, wherein R⁶ is fluorine or chlorine. Provided herein as embodiment 133 is the compound according to embodiment 131, wherein R⁶ is methyl or ethyl. Provided herein as embodiment 134 is the compound according to embodiment 131, wherein R⁶ is hydroxyl. Provided herein as embodiment 135 is the compound according to embodiment 131, wherein R⁶ is trifluoromethyl or difluoromethyl.

Provided herein as embodiment 136 is the compound according to embodiment 127, wherein R³ is aryl substituted with two occurrences of R⁶.

Provided herein as embodiment 137 is the compound according to embodiment 136, wherein R³ is naphthyl substituted with 2 occurrences of R⁶. Provided herein as embodiment 138 is the compound according to embodiment 137, wherein each occurrence of R⁶ is hydroxyl, C₂₋₄ alkynyl, C₁₋₄ alkyl, halogen, C₂₋₄ alkenyl, cyano or amino. Provided herein as embodiment 139 is the compound according to embodiment 138, wherein each occurrence of R⁶ is hydroxyl, 2-ethynyl, methyl, ethyl, fluorine, chlorine, 2-ethenyl, cyano or amino (—NH₂).

Provided herein as embodiment 140 is the compound according to embodiment 136, wherein R³ is phenyl substituted with 2 occurrences of R⁶. Provided herein as embodiment 141 is the compound according to embodiment 140, wherein each occurrence of R⁶ is C₁₋₄ haloalkyl, C₁₋₄ alkyl, halogen, C₃₋₇ cycloalkyl or amino. Provided herein as embodiment 142 is the compound according to embodiment 141, wherein each occurrence of R⁶ is trifluoromethyl, methyl, chlorine, cyclopropyl or amino (—NH₂).

Provided herein as embodiment 143 is the compound according to embodiment 127, wherein R³ is aryl substituted with 3 occurrences of R⁶.

Provided herein as embodiment 144 is the compound according to embodiment 143, wherein R³ is naphthyl substituted with 3 occurrences of R⁶. Provided herein as embodiment 145 is the compound according to embodiment 144, wherein each occurrence of R⁶ is hydroxyl, C₂₋₄ alkynyl, C₁₋₄ alkyl or halogen. Provided herein as embodiment 146 is the compound according to embodiment 145, wherein each occurrence of R⁶ is hydroxyl, 2-ethynyl, methyl, ethyl, fluorine or chlorine.

Provided herein as embodiment 147 is the compound according to embodiment 143, wherein R³ is phenyl substituted with 3 occurrences of R⁶. Provided herein as embodiment 148 is the compound according to embodiment 147, wherein each occurrence of R⁶ is halogen, hydroxyl, C₁₋₄ alkyl, C₃₋₇ cycloalkyl, cyano or amino. Provided herein as embodiment 149 is the compound according to embodiment 148, wherein each occurrence of R⁶ is hydroxyl, 2-ethynyl, cyclopropyl, methyl, ethyl, fluorine, chlorine, cyano or amino.

Provided herein as embodiment 150 is the compound according to any one of embodiments 1-126, wherein R³ is heteroaryl substituted with 0-3 occurrences of R⁶. Provided herein as embodiment 151 is the compound according to embodiment 150, wherein R³ is 2-pyridinyl, 8-quinolinyl, 5-quinolinyl, 4-isoquinolinyl, 1-isoquinolinyl, 8-isoquinolinyl, 4-(1H-indazolyl) or 7-(1H-indazolyl) substituted with 0-3 occurrences of R⁶.

Provided herein as embodiment 152 is the compound according to embodiment 151, wherein R³ is 2-pyridinyl substituted with 0-3 occurrences of R⁶. Provided herein as embodiment 153 is the compound according to embodiment 152, wherein R³ is 2-pyridinyl substituted with 2 occurrences of R⁶. Provided herein as embodiment 154 is the compound according to embodiment 153, wherein R⁶ is amino, C₁₋₄ haloalkyl or C₃₋₇ cycloalkyl. Provided herein as embodiment 155 is the compound according to embodiment 154, wherein R⁶ is amino, trifluoromethyl or cyclopropyl.

Provided herein as embodiment 156 is the compound according to embodiment 152, wherein R³ is 2-pyridinyl substituted with 3 occurrences of R⁶. Provided herein as embodiment 157 is the compound according to embodiment 156, wherein R⁶ is amino, halogen, C₁₋₄ haloalkyl or C₃₋₇ cycloalkyl. Provided herein as embodiment 158 is the compound according to embodiment 157, wherein R⁶ is amino, trifluoromethyl, methyl or cyclopropyl.

Provided herein as embodiment 159 is the compound according to embodiment 151, wherein R³ is 8-quinolinyl substituted with 0-3 occurrences of R⁶. Provided herein as embodiment 160 is the compound according to embodiment 159, wherein R³ is 8-quinolinyl substituted with 1 occurrence of R⁶ wherein R⁶ is hydroxyl.

Provided herein as embodiment 161 is the compound according to embodiment 151, wherein R³ is 5-quinolinyl substituted with 0-3 occurrences of R⁶. Provided herein as embodiment 162 is the compound according to embodiment 161, wherein R³ is 5-quinolinyl substituted with 1 occurrence of R⁶ wherein R⁶ is hydroxyl. Provided herein as embodiment 162 is the compound according to embodiment 161, wherein R³ is 5-quinolinyl substituted with 1 occurrence of R⁶ wherein R⁶ is oxo.

Provided herein as embodiment 163 is the compound according to embodiment 151, wherein R³ is 4-isoquinolinyl substituted with 0-3 occurrences of R⁶. Provided herein as embodiment 164 is the compound according to embodiment 163, wherein R³ is 4-isoquinolinyl substituted with 1 occurrence of R⁶ wherein R⁶ is halogen (e.g., chlorine).

Provided herein as embodiment 165 is the compound according to embodiment 151, wherein R³ 8-isoquinolinyl substituted with 0-3 occurrences of R⁶. Provided herein as embodiment 166 is the compound according to embodiment 165, wherein R³ 8-isoquinolinyl substituted with 1 occurrence of R⁶ wherein R⁶ is hydroxyl.

Provided herein as embodiment 167 is the compound according to embodiment 151, wherein R³ is 1-isoquinolinyl substituted with 0-3 occurrences of R⁶. Provided herein as embodiment 168 is the compound according to embodiment 167, wherein R³ is 1-isoquinolinyl is substituted with 1 occurrence of R⁶ wherein R⁶ is amino (—NH₂).

Provided herein as embodiment 169 is the compound according to embodiment 151, wherein R³ is 4-(1H-indazolyl) substituted with 0-3 occurrences of R⁶. Provided herein as embodiment 170 is the compound according to embodiment 169, wherein R³ is 4-(1H-indazolyl) is substituted with 1 occurrence of R⁶. Provided herein as embodiment 171 is the compound according to embodiment 170, wherein R⁶ is C₁₋₄ alkyl, C₃₋₇ cycloalkyl, halo, C₁₋₄ haloalkyl, cyano or C₁₋₄ alkenyl. Provided herein as embodiment 172 is the compound according to embodiment 171, wherein R⁶ is methyl, cyclopropyl, chlorine, trifluoromethyl, cyano or 2-methylprop-1-enyl.

Provided herein as embodiment 173 is the compound according to embodiment 169, wherein R³ is 4-(1H-indazolyl) substituted with 2 occurrences of R⁶. Provided herein as embodiment 174 is the compound according to embodiment 173, wherein each R⁶ is C₁₋₄ alkyl, halogen or amino. Provided herein as embodiment 175 is the compound according to embodiment 174, wherein each R⁶ is methyl, chlorine or amino.

Provided herein as embodiment 176 is the compound according to embodiment 151, wherein R³ is 7-(1H-indazolyl) substituted with 0-3 occurrences of R⁶. Provided herein as embodiment 177 is the compound according to embodiment 176, wherein R³ is 7-(1H-indazolyl) is substituted with one occurrence of R⁶. Provided herein as embodiment 178 is the compound according to embodiment 177, wherein R⁶ is C₁₋₄ alkyl (e.g., methyl).

Provided herein as embodiment 179 is the compound according to embodiment 176, wherein R³ is 7-(1H-indazolyl) substituted with 2 occurrences of R⁶. Provided herein as embodiment 180 is the compound according to embodiment 179, wherein both R⁶ are C₁₋₄ alkyl (e.g., methyl). Provided herein as embodiment 181 is the compound according to embodiment 180, wherein one R⁶ is halo (e.g., chloro) and the other R⁶ is C₁₋₄ alkyl (e.g., methyl).

Provided herein as embodiment 182 is the compound according to embodiment 176, wherein R³ is 7-(1H-indazolyl) substituted with 3 occurrences of R⁶. Provided herein as embodiment 183 is the compound according to embodiment 182, wherein all three R⁶ are C₁₋₄ alkyl (e.g., methyl).

Provided herein as embodiment 184 is the compound according to any one of embodiments 1-183 wherein R³ is

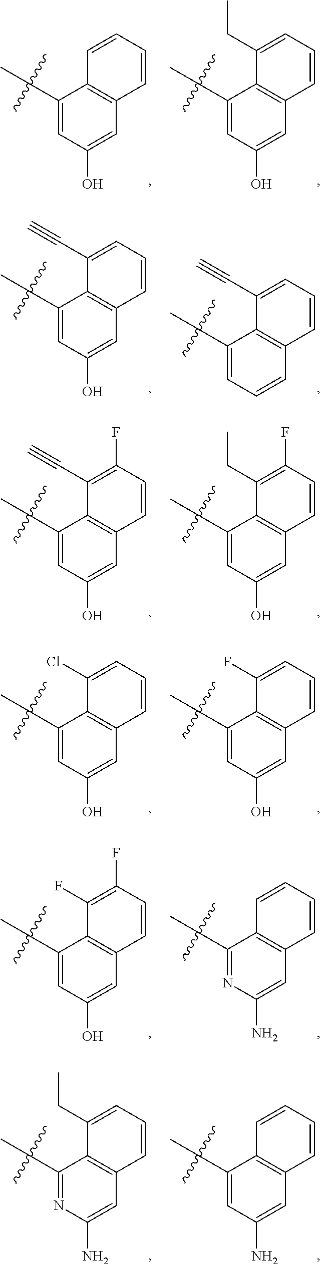

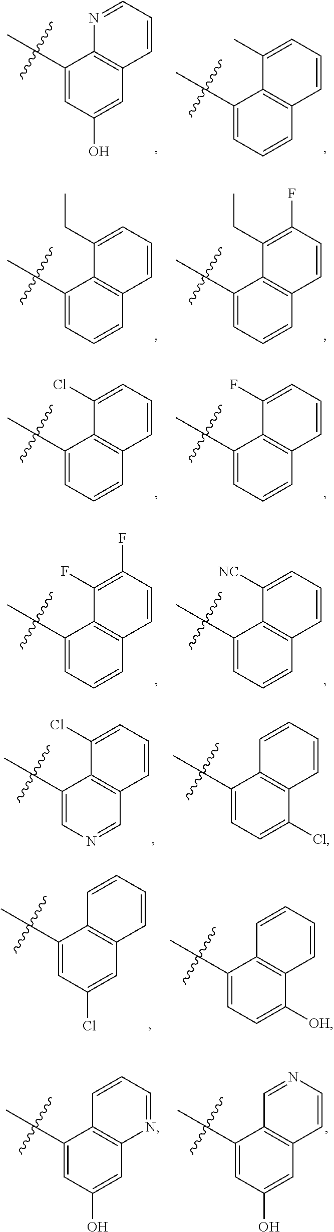

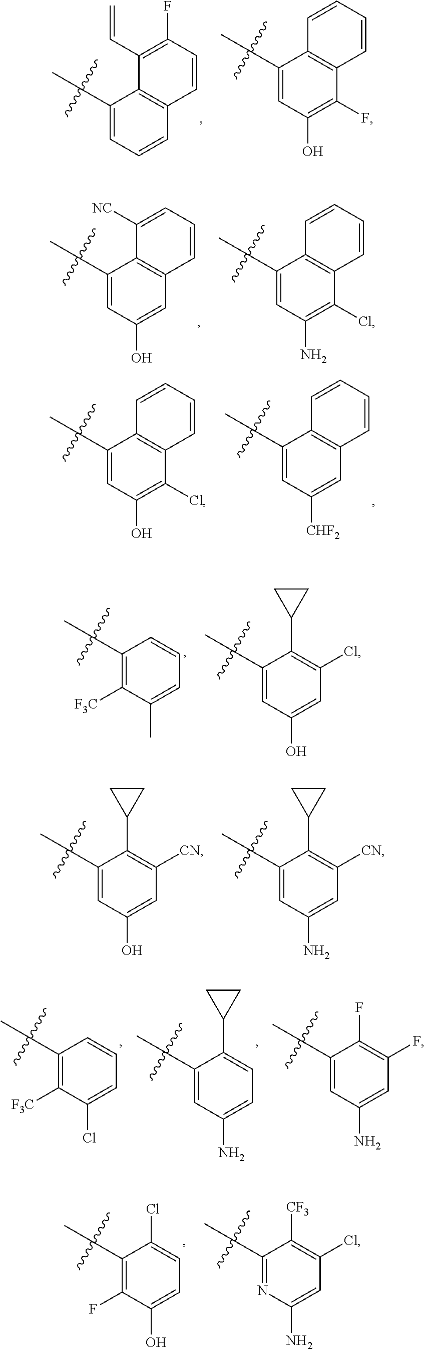

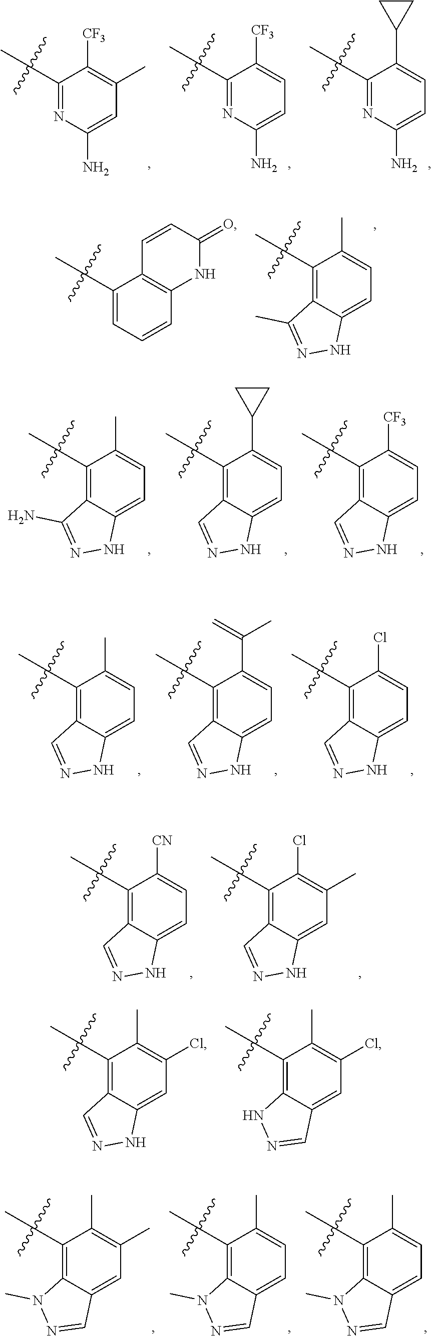

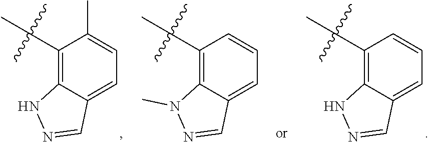

Provided herein as embodiment 185 is the compound according to embodiment 184, wherein R³ is

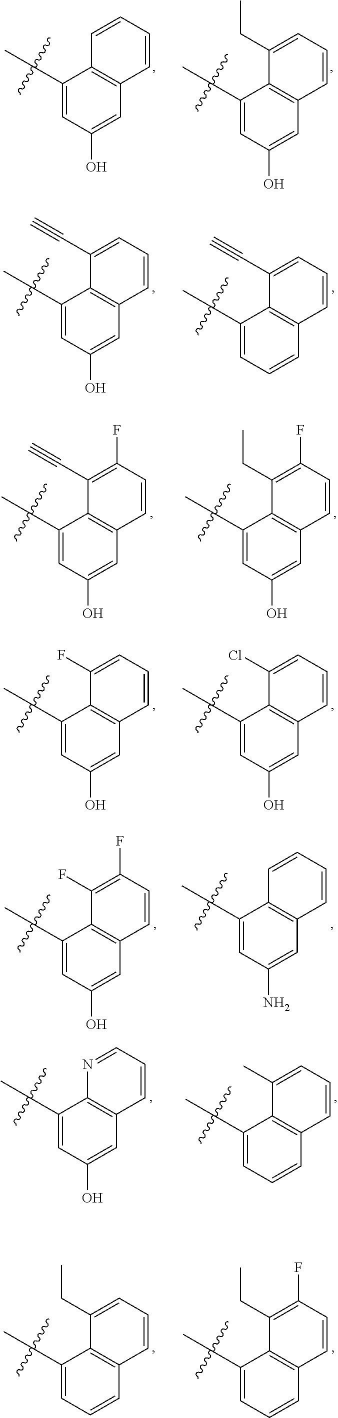

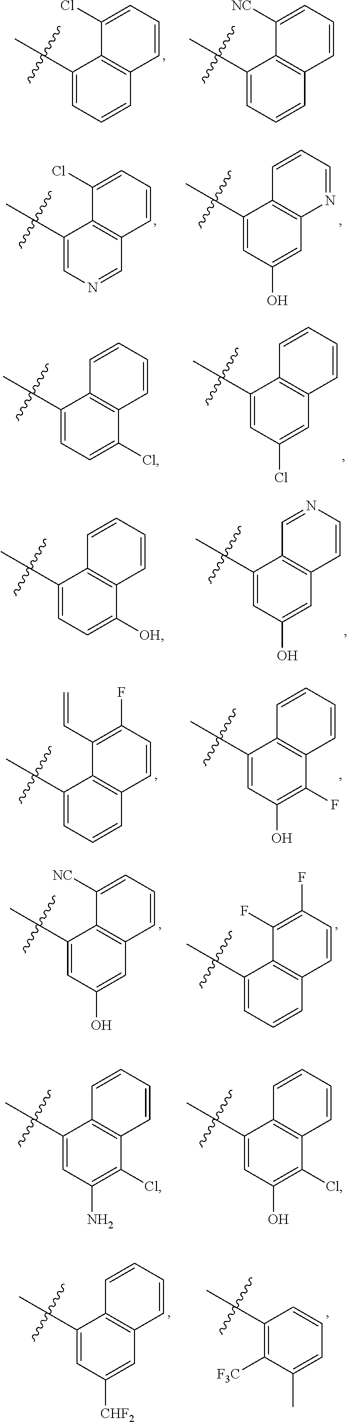

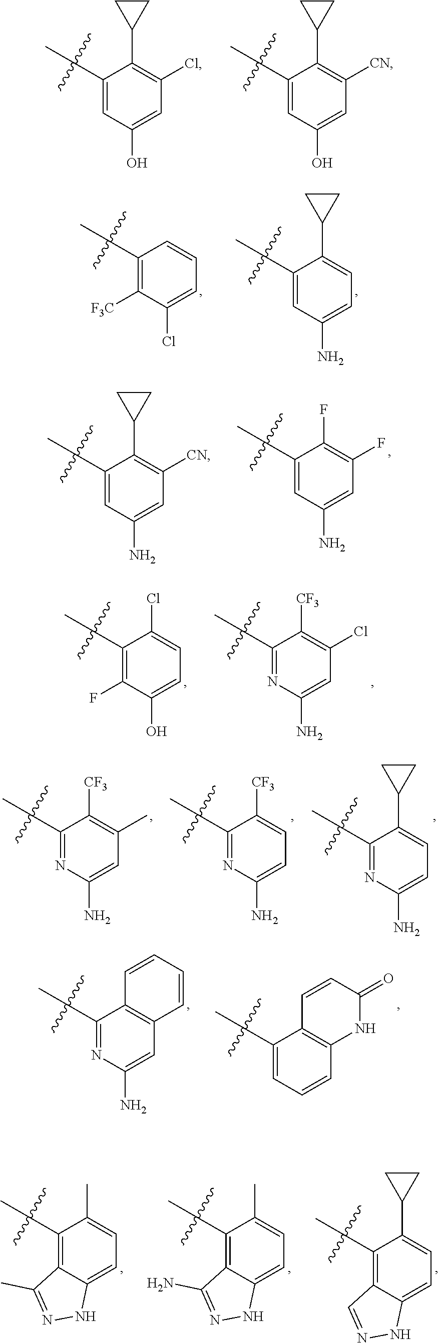

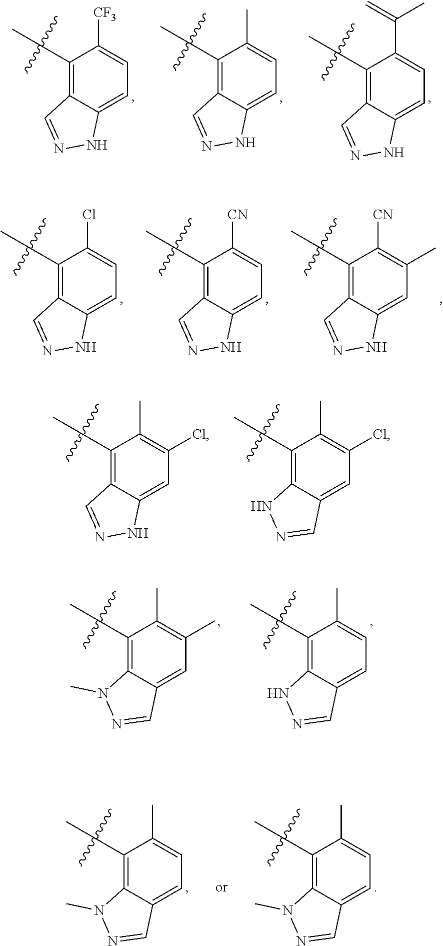

Provided herein as embodiment 186 is the compound according to embodiment 185, wherein R³ is

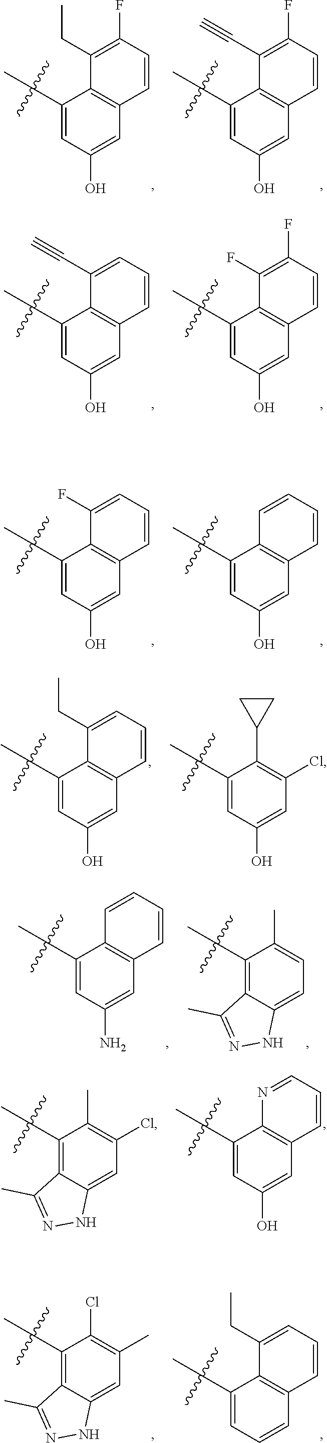

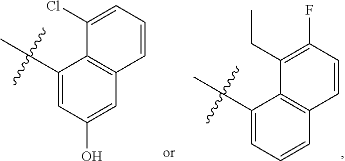 [SMILES: *c1cc(O)cc2cccc(Cl)c12 ; *c1cccc2ccc(F)c(CC)c12]

Provided herein as embodiment 187 is the compound according to embodiment 186, wherein R³ is

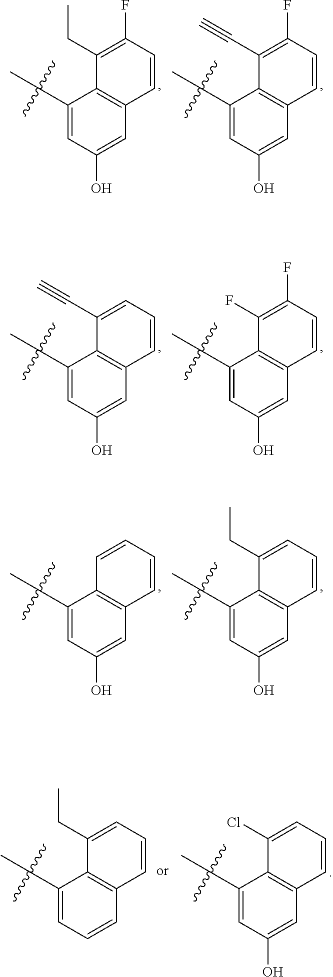

Provided herein as embodiment 188 is the compound according to embodiment 184, wherein R³ is

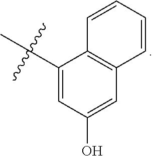 [SMILES: *c1cc(O)cc2ccccc12 ; "."]

Provided herein as embodiment 189 is the compound according to embodiment 184, wherein R³ is

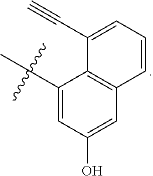 [SMILES: *c1cc(O)cc2cccc(C#C)c12 ; "."]

Provided herein as embodiment 190 is the compound according to embodiment 184, wherein R³ is

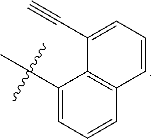 [SMILES: *c1cccc2cccc(C#C)c12 ; "."]

Provided herein as embodiment 191 is the compound according to embodiment 184, wherein R³ is

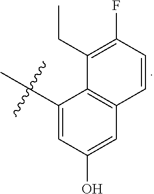 [SMILES: *c1cc(O)cc2ccc(F)c(CC)c12]

Provided herein as embodiment 192 is the compound according to embodiment 184, wherein R³ is

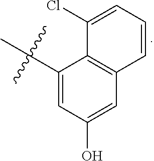 [SMILES: *c1cc(O)cc2cccc(Cl)c12]

Provided herein as embodiment 193 is the compound according to embodiment 184, wherein R³ is

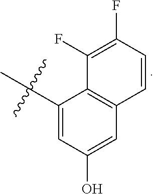 [SMILES: *c1cc(O)cc2ccc(F)c(F)c12]

Provided herein as embodiment 194 is the compound according to embodiment 184, wherein R³ is

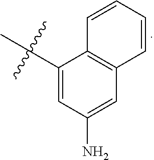 [SMILES: *c1cc(N)cc2ccccc12]

Provided herein as embodiment 195 is the compound according to embodiment 184, wherein R³ is

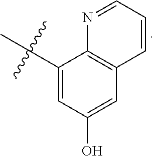 [SMILES: *c1cc(O)cc2cccnc12]

Provided herein as embodiment 196 is the compound according to embodiment 184, wherein R³ is

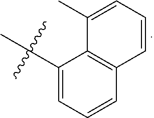 [SMILES: *c1cccc2cccc(C)c12]

Provided herein as embodiment 197 is the compound according to embodiment 184, wherein R³ is

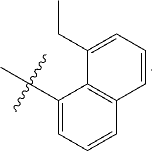 [SMILES: *c1cccc2cccc(CC)c12]

Provided herein as embodiment 198 is the compound according to embodiment 184, wherein R³ is

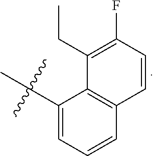 [SMILES: *c1cccc2ccc(F)c(CC)c12]

Provided herein as embodiment 199 is the compound according to embodiment 184, wherein R³ is

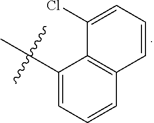 [SMILES: *c1cccc2cccc(Cl)c12]

Provided herein as embodiment 200 is the compound according to embodiment 184, wherein R³ is

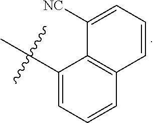 [SMILES: *c1cccc2cccc(C#N)c12]

Provided herein as embodiment 201 is the compound according to embodiment 184, wherein R³ is

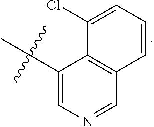 [SMILES: *c1cncc2cccc(Cl)c12]

Provided herein as embodiment 202 is the compound according to embodiment 184, wherein R³ is

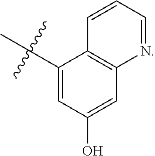 [SMILES: *c1cc(O)cc2ncccc12]

Provided herein as embodiment 203 is the compound according to embodiment 184, wherein R³ is

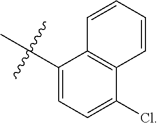 [SMILES: *c1ccc(Cl)c2ccccc12]

Provided herein as embodiment 204 is the compound according to embodiment 184, wherein R³ is

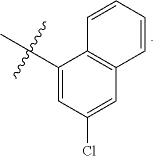 [SMILES: *c1cc(Cl)cc2ccccc12]

Provided herein as embodiment 205 is the compound according to embodiment 184, wherein R³ is

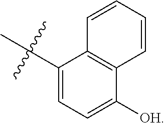 [SMILES: *c1ccc(O)c2ccccc12]

Provided herein as embodiment 206 is the compound according to embodiment 184, wherein R³ is

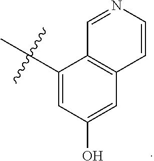 [SMILES: *c1cc(O)cc2ccncc12]

Provided herein as embodiment 207 is the compound according to embodiment 184, wherein R³ is

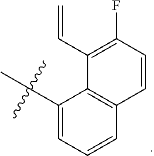 [SMILES: *c1cccc2ccc(F)c(C=C)c12]

Provided herein as embodiment 208 is the compound according to embodiment 184, wherein R³ is

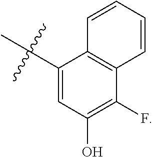 [SMILES: *c1cc(O)c(F)c2ccccc12]

Provided herein as embodiment 209 is the compound according to embodiment 184, wherein R³ is

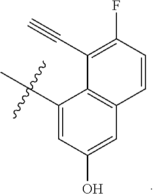 [SMILES: *c1cc(O)cc2ccc(F)c(C#C)c12]

Provided herein as embodiment 210 is the compound according to embodiment 184, wherein R³ is

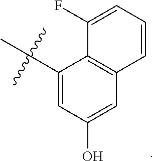 [SMILES: *c1cc(O)cc2cccc(F)c12]

Provided herein as embodiment 211 is the compound according to embodiment 184, wherein R³ is

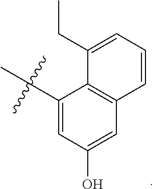 [SMILES: *c1cc(O)cc2cccc(CC)c12]

Provided herein as embodiment 212 is the compound according to embodiment 184, wherein R³ is

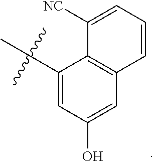 [SMILES: *c1cc(O)cc2cccc(C#N)c12]

Provided herein as embodiment 213 is the compound according to embodiment 184, wherein R³ is

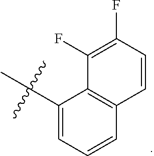 [SMILES: *c1cccc2ccc(F)c(F)c12]

Provided herein as embodiment 214 is the compound according to embodiment 184, wherein R³ is

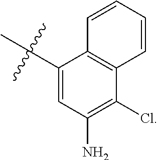 [SMILES: *c1cc(N)c(Cl)c2ccccc12]

Provided herein as embodiment 215 is the compound according to embodiment 184, wherein R³ is

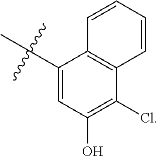 [SMILES: *c1cc(O)c(Cl)c2ccccc12]

Provided herein as embodiment 216 is the compound according to embodiment 184, wherein R³ is

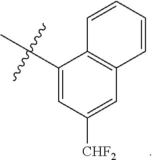 [SMILES: *c1cc(C(F)F)cc2ccccc12]

Provided herein as embodiment 217 is the compound according to embodiment 184, wherein R³ is

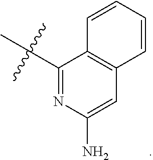 [SMILES: *c1nc(N)cc2ccccc12]

Provided herein as embodiment 218 is the compound according to embodiment 184, wherein R³ is

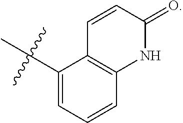 [SMILES: *c1cccc2[nH]c(=O)ccc12]

Provided herein as embodiment 219 is the compound according to embodiment 184, wherein R³ is

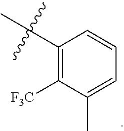 [SMILES: *c1cccc(C)c1C(F)(F)F]

Provided herein as embodiment 220 is the compound according to embodiment 184, wherein R³ is

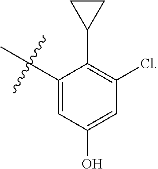 [SMILES: *c1cc(O)cc(Cl)c1C1CC1]

Provided herein as embodiment 221 is the compound according to embodiment 184, wherein R³ is

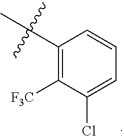 [SMILES: *c1cccc(Cl)c1C(F)(F)F ; "."]

Provided herein as embodiment 222 is the compound according to embodiment 184, wherein R³ is

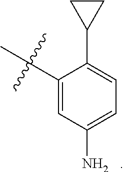 [SMILES: *c1cc(N)ccc1C1CC1 ; "."]

Provided herein as embodiment 223 is the compound according to embodiment 184, wherein R³ is

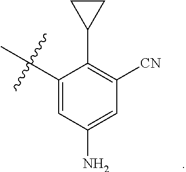 [SMILES: *c1cc(N)cc(C#N)c1C1CC1 ; "."]

Provided herein as embodiment 224 is the compound according to embodiment 184, wherein R³ is

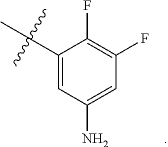 [SMILES: *c1cc(N)cc(F)c1F ; "."]

Provided herein as embodiment 225 is the compound according to embodiment 184, wherein R³ is

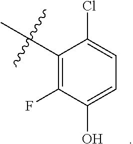 [SMILES: *c1c(Cl)ccc(O)c1F ; "."]

Provided herein as embodiment 226 is the compound according to embodiment 184, wherein R³ is

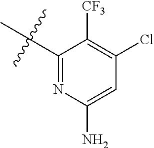 [SMILES: *c1nc(N)cc(Cl)c1C(F)(F)F]

Provided herein as embodiment 227 is the compound according to embodiment 184, wherein R³ is

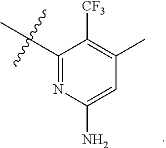 [SMILES: *c1nc(N)cc(C)c1C(F)(F)F]

Provided herein embodiment 228 is the compound according to embodiment 184, wherein R³ is

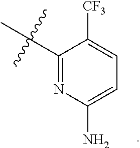 [SMILES: *c1nc(N)ccc1C(F)(F)F]

Provided herein as embodiment 229 is the compound according to embodiment 184, wherein R³ is

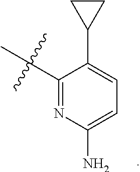 [SMILES: *c1nc(N)ccc1C1CC1]

Provided herein as embodiment 230 is the compound according to embodiment 184, wherein R³ is

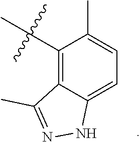 [SMILES: *c1c(C)ccc2[nH]nc(C)c12]

Provided herein as embodiment 231 is the compound according to embodiment 184, wherein R³ is

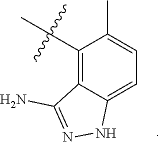 [SMILES: *c1c(C)ccc2[nH]nc(N)c12]

Provided herein as embodiment 232 is the compound according to embodiment 184, wherein R³ is

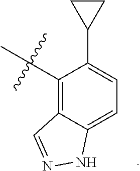 [SMILES: *c1c(C2CC2)ccc2[nH]ncc12]

Provided herein as embodiment 233 is the compound according to embodiment 184, wherein R³ is

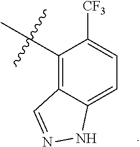 [SMILES: *c1c(C(F)(F)F)ccc2[nH]ncc12]

Provided herein as embodiment 234 is the compound according to embodiment 184, wherein R³ is

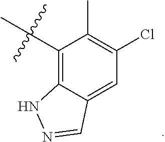 [SMILES: *c1c(C)c(Cl)cc2cn[nH]c12]

Provided herein as embodiment 235 is the compound according to embodiment 184, wherein R³ is

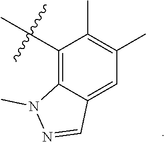

Provided herein as embodiment 236 is the compound according to embodiment 184, wherein R³ is

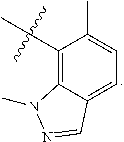 [SMILES: *c1c(C)ccc2cnn(C)c12]

Provided herein as embodiment 237 is the compound according to embodiment 184, wherein R³ is

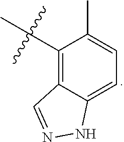 [SMILES: *c1c(C)ccc2[nH]ncc12]

Provided herein as embodiment 238 is the compound according to embodiment 184, wherein R³ is

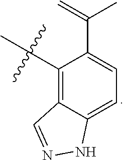 [SMILES: *c1c(C(=C)C)ccc2[nH]ncc12]

Provided herein as embodiment 239 is the compound according to embodiment 184, wherein R³ is

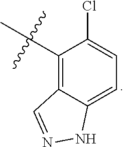 [SMILES: *c1c(Cl)ccc2[nH]ncc12]

Provided herein as embodiment 240 is the compound according to embodiment 184, wherein R³ is

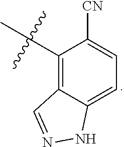 [SMILES: *c1c(C#N)ccc2[nH]ncc12]

Provided herein as embodiment 241 is the compound according to embodiment 184, wherein R³ is

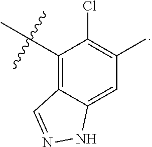 [SMILES: *c1c(Cl)c(C)cc2[nH]ncc12]

Provided herein as embodiment 242 is the compound according to embodiment 184, wherein R³ is

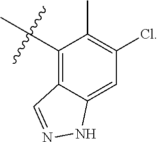 [SMILES: *c1c(C)c(Cl)cc2[nH]ncc12]

Provided herein as embodiment 243 is the compound according to embodiment 184, wherein R³ is

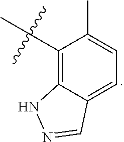 [SMILES: *c1c(C)ccc2cn[nH]c12]

Provided herein as embodiment 244 is the compound according to embodiment 184, wherein R³ is

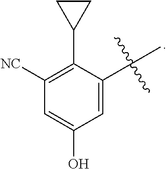 [SMILES: *c1cc(O)cc(C#N)c1C1CC1]

Provided herein as embodiment 245 is the compound according to any one of embodiments 1-244, wherein R² is hydrogen. Provided herein as embodiment 246 is the compound according to any one of embodiments 1-244, wherein R² is halogen (e.g., fluorine or chlorine). Provided herein as embodiment 247 is the compound according to any one of embodiments 1-244, wherein R² is C₁₋₄ alkyl (e.g., methyl or ethyl). Provided herein as embodiment 248 is the compound according to any one of embodiments 1-244, wherein R² is C₂₋₄ alkenyl (e.g., 2-ethenyl).

Provided herein as embodiment 249 is the compound according to any one of embodiments 1-248, wherein R⁴ is halogen (e.g., fluorine). Provided herein as embodiment 250 is the compound according to any one of embodiments 1-248, wherein R⁴ is hydrogen. Provided herein as embodiment 251 is the compound according to any one of embodiments 1-248, wherein R⁴ is hydroxyl. Provided herein as embodiment 252 is the compound according to any one of embodiments 1-248, wherein R² is C₁₋₄ alkyl (e.g., methyl).

Provided herein as embodiment 253 is the compound according to any one of embodiments 1-252, wherein R⁷ is hydrogen. Provided herein as embodiment 254 is the compound according to any one of embodiments 1-252, wherein R⁷ is methyl. Provided herein as embodiment 255 is the compound according to embodiments 1-252, wherein R⁷ is fluorine.

Provided herein as embodiment 256 is the compound according to any one of embodiments 1-255, wherein R³ is not benzo[d]thiazole. Provided herein as embodiment 257 is the compound according to any one of embodiments 1-255, wherein R³ is not 2-aminobenzo[d]thiazole.

Provided herein as embodiment 258 is the compound according to embodiment 1, wherein the compound is a compound of Formula (III):

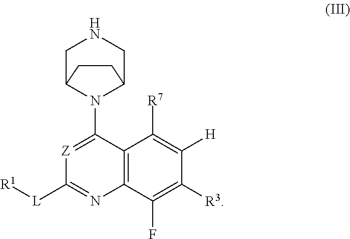

Provided herein as embodiment 259 is the compound according to embodiment 1, wherein the compound is a compound of Formula (IV):

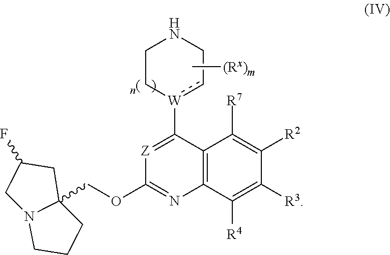

Provided herein as embodiment 260 is the compound according to embodiment 1, wherein the compound is a compound of Formula (V):

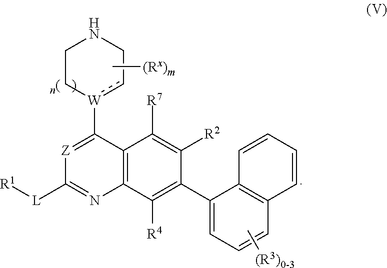

Provided herein as embodiment 261 is the compound according to embodiment 1, wherein the compound is a compound of Formula (VI):

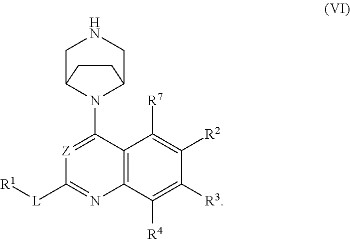

Provided herein as embodiment 262 is the compound according to embodiment 1, wherein the compound is a compound of Formula (VII):

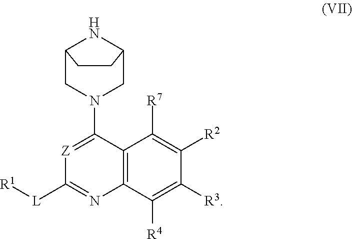

Provided herein as embodiment 263 is the compound according to embodiment 1, wherein the compound is a compound of Formula (VIII):

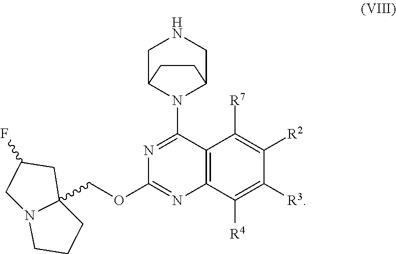

Provided herein as embodiment 264 is the compound according to embodiment 1, wherein the compound is a compound of Formula (IX):

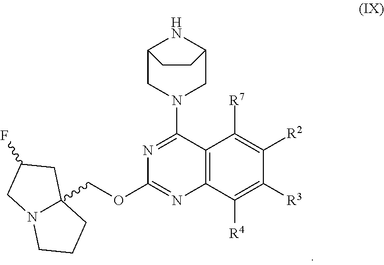

Provided herein as embodiment 265 is the compound according to embodiment 1, wherein the compound is a compound of Formula (X):

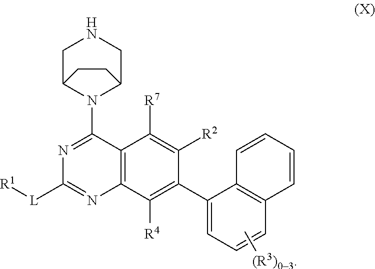

Provided herein as embodiment 266 is the compound according to embodiment 1, wherein the compound is a compound of Formula (XI):

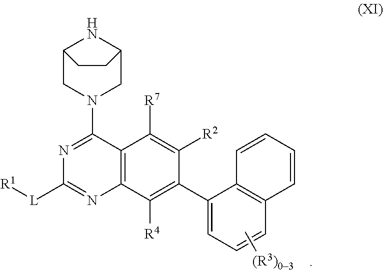

Provided herein as embodiment 267 is the compound according to embodiment 1, wherein the compound is selected from one of the following compounds:
4-(4-((1R,5S)-3,8-diazabicyclo[3.2.1]octan-3-yl)-6,8-difluoro-2-(((2R,7aS)-2-fluorotetrahydro-1H-pyrrolizin-7a(5H)-yl)methoxy)quinazolin-7-yl)-5-ethynyl-6-fluoronaphthalen-2-ol;
4-(4-((1R,5S)-3,8-diazabicyclo[3.2.1]octan-3-yl)-6,8-difluoro-2-(((2R,7aS)-2-fluorotetrahydro-1H-pyrrolizin-7a(5H)-yl)methoxy)quinazolin-7-yl)-5-ethynylnaphthalen-2-ol;
4-(4-((1R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)quinazolin-7-yl)-5-ethynyl-6-fluoronaphthalen-2-ol;
4-(4-((1R,5S)-3,8-diazabicyclo[3.2.1]octan-3-yl)-6,8-difluoro-2-(((2R,7aS)-2-fluorotetrahydro-1H-pyrrolizin-7a(5H)-yl)methoxy)quinazolin-7-yl)-5-ethyl-6-fluoronaphthalen-2-ol;
4-(4-((1S,4S)-2,5-diazabicyclo[2.2.2]octan-2-yl)-6,8-difluoro-2-(((2R,7aS)-2-fluorotetrahydro-1H-pyrrolizin-7a(5H)-yl)methoxy)quinazolin-7-yl)-5-ethyl-6-fluoronaphthalen-2-ol;
4-(4-((1S,4S)-2,5-diazabicyclo[2.2.2]octan-2-yl)-8-fluoro-2-(((2R,7aS)-2-fluorotetrahydro-1H-pyrrolizin-7a(5H)-yl)methoxy)quinazolin-7-yl)-5-ethynyl-6-fluoronaphthalen-2-ol;
4-(4-((1R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)quinazolin-7-yl)-5-ethyl-6-fluoronaphthalen-2-ol;
4-(4-((1R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)quinazolin-7-yl)-5-ethynylnaphthalen-2-ol;
4-(4-((1R,5S)-3,8-diazabicyclo[3.2.1]octan-8-yl)-8-fluoro-2-(((2R,7aS)-2-fluorotetrahydro-1H-pyrrolizin-7a(5H)-yl)methoxy)quinazolin-7-yl)-5-ethynyl-6-fluoronaphthalen-2-ol;
4-(4-((1S,4S)-2,5-diazabicyclo[2.2.2]octan-2-yl)-6,8-difluoro-2-(((2R,7aS)-2-fluorotetrahydro-1H-pyrrolizin-7a(5H)-yl)methoxy)quinazolin-7-yl)-5-ethynyl-6-fluoronaphthalen-2-ol;
4-(4-((1S,4S)-2,5-diazabicyclo[2.2.2]octan-2-yl)-6,8-difluoro-2-(((2R,7aS)-2-fluorotetrahydro-1H-pyrrolizin-7a(5H)-yl)methoxy)quinazolin-7-yl)-5-ethynylnaphthalen-2-ol;
4-(4-((1R,5S)-3,8-diazabicyclo[3.2.1]octan-3-yl)-6-fluoro-2-(((2R,7aS)-2-fluorotetrahydro-1H-pyrrolizin-7a(5H)-yl)methoxy)quinazolin-7-yl)-5-ethynyl-6-fluoronaphthalen-2-ol;
4-(4-((1R,5S)-3,8-diazabicyclo[3.2.1]octan-8-yl)-8-fluoro-2-(((2R,7aS)-2-fluorotetrahydro-1H-pyrrolizin-7a(5H)-yl)methoxy)quinazolin-7-yl)-5-ethynylnaphthalen-2-ol;
4-(4-((1R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)quinazolin-7-yl)-5,6-difluoronaphthalen-2-ol;
4-(4-((1R,5S)-3,8-diazabicyclo[3.2.1]octan-3-yl)-6,8-difluoro-2-(((2R,7aS)-2-fluorotetrahydro-1H-pyrrolizin-7a(5H)-yl)methoxy)quinazolin-7-yl)-5,6-difluoronaphthalen-2-ol;
4-(4-((1R,5S)-3,8-diazabicyclo[3.2.1]octan-3-yl)-6,8-difluoro-2-(((2R,7aS)-2-fluorotetrahydro-1H-pyrrolizin-7a(5H)-yl)methoxy)quinazolin-7-yl)-5-ethylnaphthalen-2-ol;
4-(4-((1S,4S)-2,5-diazabicyclo[2.2.2]octan-2-yl)-8-fluoro-2-(((2R,7aS)-2-fluorotetrahydro-1H-pyrrolizin-7a(5H)-yl)methoxy)quinazolin-7-yl)-5-ethylnaphthalen-2-ol;
4-(4-((1S,4S)-2,5-diazabicyclo[2.2.2]octan-2-yl)-6,8-difluoro-2-(((2R,7aS)-2-fluorotetrahydro-1H-pyrrolizin-7a(5H)-yl)methoxy)quinazolin-7-yl)-5,6-difluoronaphthalen-2-ol;
4-(4-((1S,4S)-2,5-diazabicyclo[2.2.2]octan-2-yl)-6,8-difluoro-2-(((2R,7aS)-2-fluorotetrahydro-1H-pyrrolizin-7a(5H)-yl)methoxy)quinazolin-7-yl)-5-ethylnaphthalen-2-ol;
4-(4-((1R,5S)-3,8-diazabicyclo[3.2.1]octan-3-yl)-6,8-difluoro-2-(((2R,7aS)-2-fluorotetrahydro-1H-pyrrolizin-7a(5H)-yl)methoxy)quinazolin-7-yl)naphthalen-2-ol;
4-(4-((1R,5S)-3,8-diazabicyclo[3.2.1]octan-3-yl)-6-fluoro-2-(((2R,7aS)-2-fluorotetrahydro-1H-pyrrolizin-7a(5H)-yl)methoxy)quinazolin-7-yl)-5-ethynylnaphthalen-2-ol;
4-(4-((1R,5S)-3,8-diazabicyclo[3.2.1]octan-3-yl)-2-(((2R,7aS)-2-fluorotetrahydro-1H-pyrrolizin-7a(5H)-yl)methoxy)-8-methylquinazolin-7-yl)-5-ethynylnaphthalen-2-ol;
4-(4-((1R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)quinazolin-7-yl)naphthalen-2-ol;
3-(4-((1R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)quinazolin-7-yl)-5-chloro-4-cyclopropylphenyl;
3-(4-((1R,5S)-3,8-diazabicyclo[3.2.1]octan-3-yl)-6,8-difluoro-2-(((2R,7aS)-2-fluorotetrahydro-1H-pyrrolizin-7a(5H)-yl)methoxy)quinazolin-7-yl)-5-chloro-4-cyclopropylphenyl;
4-(4-((1S,4S)-2,5-diazabicyclo[2.2.2]octan-2-yl)-8-fluoro-2-(((2R,7aS)-2-fluorotetrahydro-1H-pyrrolizin-7a(5H)-yl)methoxy)quinazolin-7-yl)-5-ethyl-6-fluoronaphthalen-2-ol;
4-((1R,5S)-3,8-diazabicyclo[3.2.1]octan-3-yl)-7-(8-ethyl-7-fluoro-3-hydroxynaphthalen-1-yl)-2-(((2R,7aS)-2-fluorotetrahydro-1H-pyrrolizin-7a(5H)-yl)methoxy)quinazolin-6-ol;
3-(4-((1S,4S)-2,5-diazabicyclo[2.2.2]octan-2-yl)-6,8-difluoro-2-(((2R,7aS)-2-fluorotetrahydro-1H-pyrrolizin-7a(5H)-yl)methoxy)quinazolin-7-yl)-5-chloro-4-cyclopropylphenyl;
4-(4-((1S,4S)-2,5-diazabicyclo[2.2.2]octan-2-yl)-6,8-difluoro-2-(((2R,7aS)-2-fluorotetrahydro-1H-pyrrolizin-7a(5H)-yl)methoxy)quinazolin-7-yl)-5,6-difluoronaphthalen-2-ol;
5-ethyl-6-fluoro-4-(8-fluoro-4-(1-fluoro-3,8-diazabicyclo[3.2.1]octan-3-yl)-2-(((2R,7aS)-2-fluorotetrahydro-1H-pyrrolizin-7a(5H)-yl)methoxy)quinazolin-7-yl)naphthalen-2-ol;
4-(4-((1R,5S)-3,8-diazabicyclo[3.2.1]octan-3-yl)-6,8-difluoro-2-(((2R,7aS)-2-fluorotetrahydro-1H-pyrrolizin-7a(5H)-yl)methoxy)quinazolin-7-yl)-5-chloronaphthalen-2-ol;
4-(4-((1R,5S)-3,8-diazabicyclo[3.2.1]octan-3-yl)-2-(((2R,7aS)-2-fluorotetrahydro-1H-pyrrolizin-7a(5H)-yl)methoxy)quinazolin-7-yl)-5-ethyl-6-fluoronaphthalen-2-ol;
4-((1R,5S)-3,8-diazabicyclo[3.2.1]octan-8-yl)-7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)quinazolin-6-ol;
4-(4-((1R,5S)-3,8-diazabicyclo[3.2.1]octan-3-yl)-2-(((2R,7aS)-2-fluorotetrahydro-1H-pyrrolizin-7a(5H)-yl)methoxy)quinazolin-7-yl)-5,6-difluoronaphthalen-2-ol;
4-(4-((1S,4S)-2,5-diazabicyclo[2.2.2]octan-2-yl)-6-fluoro-2-(((2R,7aS)-2-fluorotetrahydro-1H-pyrrolizin-7a(5H)-yl)methoxy)quinazolin-7-yl)-5-ethyl-6-fluoronaphthalen-2-ol;
4-(4-((1R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)-5-methylquinazolin-7-yl)-5-ethyl-6-fluoronaphthalen-2-ol;
4-(4-((1R,5S)-3,8-diazabicyclo[3.2.1]octan-3-yl)-5,8-difluoro-2-(((2R,7aS)-2-fluorotetrahydro-1H-pyrrolizin-7a(5H)-yl)methoxy)quinazolin-7-yl)-5-ethyl-6-fluoronaphthalen-2-ol;
4-(4-((1S,4S)-2,5-diazabicyclo[2.2.2]octan-2-yl)-8-fluoro-2-(((2R,7aS)-2-fluorotetrahydro-1H-pyrrolizin-7a(5H)-yl)methoxy)quinazolin-7-yl)-5,6-difluoronaphthalen-2-ol;
4-((1R,5S)-3,8-diazabicyclo[3.2.1]octan-3-yl)-7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)quinazolin-6-ol;
4-(4-((1R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)quinazolin-7-yl)-5-ethylnaphthalen-2-ol;
4-(4-((1R,5S)-3,8-diazabicyclo[3.2.1]octan-3-yl)-6-chloro-8-fluoro-2-(((2R,7aS)-2-fluorotetrahydro-1H-pyrrolizin-7a(5H)-yl)methoxy)quinazolin-7-yl)-5-ethyl-6-fluoronaphthalen-2-ol;
4-(4-((1R,5S)-3-oxa-7,9-diazabicyclo[3.3.1]nonan-9-yl)-8-fluoro-2-(((2R,7aS)-2-fluorotetrahydro-1H-pyrrolizin-7a(5H)-yl)methoxy)quinazolin-7-yl)-5-ethyl-6-fluoronaphthalen-2-ol;
4-(4-((1R,5S)-3,8-diazabicyclo[3.2.1]octan-3-yl)-6-fluoro-2-(((2R,7aS)-2-fluorotetrahydro-1H-pyrrolizin-7a(5H)-yl)methoxy)quinazolin-7-yl)-5-ethyl-6-fluoronaphthalen-2-ol;
4-(4-((1R,5S)-3,8-diazabicyclo[3.2.1]octan-3-yl)-6,8-difluoro-2-(((2R,7aS)-2-fluorotetrahydro-1H-pyrrolizin-7a(5H)-yl)methoxy)quinazolin-7-yl)-5-fluoronaphthalen-2-ol;
4-(4-((1S,4S)-2,5-diazabicyclo[2.2.2]octan-2-yl)-6-chloro-8-fluoro-2-(((2R,7aS)-2-fluorotetrahydro-1H-pyrrolizin-7a(5H)-yl)methoxy)quinazolin-7-yl)-5-ethyl-6-fluoronaphthalen-2-ol;
4-(4-((1R,5S)-3,8-diazabicyclo[3.2.1]octan-3-yl)-6,8-difluoro-2-(((2R,7aR)-2-fluorotetrahydro-1H-pyrrolizin-7a(5H)-yl)methoxy)quinazolin-7-yl)naphthalen-2-ol;
4-(4-((1S,4S)-2,5-diazabicyclo[2.2.2]octan-2-yl)-2-(((2R,7aS)-2-fluorotetrahydro-1H-pyrrolizin-7a(5H)-yl)methoxy)quinazolin-7-yl)-5-ethyl-6-fluoronaphthalen-2-ol;
4-(4-((1R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)quinazolin-7-yl)-5-fluoronaphthalen-2-ol;
4-(4-((1R,5S)-3,8-diazabicyclo[3.2.1]octan-8-yl)-6,8-difluoro-2-(((2R,7aS)-2-fluorotetrahydro-1H-pyrrolizin-7a(5H)-yl)methoxy)quinazolin-7-yl)-5-ethyl-6-fluoronaphthalen-2-ol; or
8-(4-((1R,5S)-3,8-diazabicyclo[3.2.1]octan-3-yl)-6,8-difluoro-2-(((2R,7aS)-2-fluorotetrahydro-1H-pyrrolizin-7a(5H)-yl)methoxy)quinazolin-7-yl)-6-hydroxy-1-naphthonitrile.

Provided herein as embodiment 268 is the compound according to embodiment 1, wherein the compound is selected from one of the following compounds:
4-(4-((1R,5S)-3,8-diazabicyclo[3.2.1]octan-3-yl)-6,8-difluoro-2-(((2R,7aS)-2-fluorotetrahydro-1H-pyrrolizin-7a(5H)-yl)methoxy)quinazolin-7-yl)-5-ethynyl-6-fluoronaphthalen-2-ol;
4-(4-((1R,5S)-3,8-diazabicyclo[3.2.1]octan-3-yl)-6,8-difluoro-2-(((2R,7aS)-2-fluorotetrahydro-1H-pyrrolizin-7a(5H)-yl)methoxy)quinazolin-7-yl)-5-ethynylnaphthalen-2-ol;
4-(4-((1R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)quinazolin-7-yl)-5-ethynyl-6-fluoronaphthalen-2-ol;
4-(4-((1R,5S)-3,8-diazabicyclo[3.2.1]octan-3-yl)-6,8-difluoro-2-(((2R,7aS)-2-fluorotetrahydro-1H-pyrrolizin-7a(5H)-yl)methoxy)quinazolin-7-yl)-5-ethyl-6-fluoronaphthalen-2-ol;
4-(4-((1S,4S)-2,5-diazabicyclo[2.2.2]octan-2-yl)-6,8-difluoro-2-(((2R,7aS)-2-fluorotetrahydro-1H-pyrrolizin-7a(5H)-yl)methoxy)quinazolin-7-yl)-5-ethyl-6-fluoronaphthalen-2-ol;
4-(4-((1S,4S)-2,5-diazabicyclo[2.2.2]octan-2-yl)-8-fluoro-2-(((2R,7aS)-2-fluorotetrahydro-1H-pyrrolizin-7a(5H)-yl)methoxy)quinazolin-7-yl)-5-ethynyl-6-fluoronaphthalen-2-ol;
4-(4-((1R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)quinazolin-7-yl)-5-ethyl-6-fluoronaphthalen-2-ol;
4-(4-((1R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)quinazolin-7-yl)-5-ethynylnaphthalen-2-ol;
4-(4-((1R,5S)-3,8-diazabicyclo[3.2.1]octan-8-yl)-8-fluoro-2-(((2R,7aS)-2-fluorotetrahydro-1H-pyrrolizin-7a(5H)-yl)methoxy)quinazolin-7-yl)-5-ethynyl-6-fluoronaphthalen-2-ol;
4-(4-((1S,4S)-2,5-diazabicyclo[2.2.2]octan-2-yl)-6,8-difluoro-2-(((2R,7aS)-2-fluorotetrahydro-1H-pyrrolizin-7a(5H)-yl)methoxy)quinazolin-7-yl)-5-ethynyl-6-fluoronaphthalen-2-ol;
4-(4-((1S,4S)-2,5-diazabicyclo[2.2.2]octan-2-yl)-6,8-difluoro-2-(((2R,7aS)-2-fluorotetrahydro-1H-pyrrolizin-7a(5H)-yl)methoxy)quinazolin-7-yl)-5-ethynylnaphthalen-2-ol;
4-(4-((1R,5S)-3,8-diazabicyclo[3.2.1]octan-3-yl)-6-fluoro-2-(((2R,7aS)-2-fluorotetrahydro-1H-pyrrolizin-7a(5H)-yl)methoxy)quinazolin-7-yl)-5-ethynyl-6-fluoronaphthalen-2-ol;
4-(4-((1R,5S)-3,8-diazabicyclo[3.2.1]octan-8-yl)-8-fluoro-2-(((2R,7aS)-2-fluorotetrahydro-1H-pyrrolizin-7a(5H)-yl)methoxy)quinazolin-7-yl)-5-ethynylnaphthalen-2-ol;
4-(4-((1R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)quinazolin-7-yl)-5,6-difluoronaphthalen-2-ol;
4-(4-((1R,5S)-3,8-diazabicyclo[3.2.1]octan-3-yl)-6,8-difluoro-2-(((2R,7aS)-2-fluorotetrahydro-1H-pyrrolizin-7a(5H)-yl)methoxy)quinazolin-7-yl)-5,6-difluoronaphthalen-2-ol;
4-(4-((1R,5S)-3,8-diazabicyclo[3.2.1]octan-3-yl)-6,8-difluoro-2-(((2R,7aS)-2-fluorotetrahydro-1H-pyrrolizin-7a(5H)-yl)methoxy)quinazolin-7-yl)-5-ethylnaphthalen-2-ol;
4-(4-((1S,4S)-2,5-diazabicyclo[2.2.2]octan-2-yl)-8-fluoro-2-(((2R,7aS)-2-fluorotetrahydro-1H-pyrrolizin-7a(5H)-yl)methoxy)quinazolin-7-yl)-5-ethylnaphthalen-2-ol;
4-(4-((1S,4S)-2,5-diazabicyclo[2.2.2]octan-2-yl)-6,8-difluoro-2-(((2R,7aS)-2-fluorotetrahydro-1H-pyrrolizin-7a(5H)-yl)methoxy)quinazolin-7-yl)-5,6-difluoronaphthalen-2-ol;
4-(4-((1S,4S)-2,5-diazabicyclo[2.2.2]octan-2-yl)-6,8-difluoro-2-(((2R,7aS)-2-fluorotetrahydro-1H-pyrrolizin-7a(5H)-yl)methoxy)quinazolin-7-yl)-5-ethylnaphthalen-2-ol;
4-(4-((1R,5S)-3,8-diazabicyclo[3.2.1]octan-3-yl)-6,8-difluoro-2-(((2R,7aS)-2-fluorotetrahydro-1H-pyrrolizin-7a(5H)-yl)methoxy)quinazolin-7-yl)naphthalen-2-ol;
4-(4-((1R,5S)-3,8-diazabicyclo[3.2.1]octan-3-yl)-6-fluoro-2-(((2R,7aS)-2-fluorotetrahydro-1H-pyrrolizin-7a(5H)-yl)methoxy)quinazolin-7-yl)-5-ethynylnaphthalen-2-ol;
4-(4-((1R,5S)-3,8-diazabicyclo[3.2.1]octan-3-yl)-2-(((2R,7aS)-2-fluorotetrahydro-1H-pyrrolizin-7a(5H)-yl)methoxy)-8-methylquinazolin-7-yl)-5-ethynylnaphthalen-2-ol;
4-(4-((1R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)quinazolin-7-yl)naphthalen-2-ol;
3-(4-((1R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)quinazolin-7-yl)-5-chloro-4-cyclopropylphenyl; or
3-(4-((1R,5S)-3,8-diazabicyclo[3.2.1]octan-3-yl)-6,8-difluoro-2-(((2R,7aS)-2-fluorotetrahydro-1H-pyrrolizin-7a(5H)-yl)methoxy)quinazolin-7-yl)-5-chloro-4-cyclopropylphenyl.

Provided herein as embodiment 269 is the compound according to embodiment 1, wherein the compound is selected from one of the following compounds:
4-(4-((1R,5S)-3,8-diazabicyclo[3.2.1]octan-3-yl)-6,8-difluoro-2-(((2R,7aS)-2-fluorotetrahydro-1H-pyrrolizin-7a(5H)-yl)methoxy)quinazolin-7-yl)-5-ethynyl-6-fluoronaphthalen-2-ol;
4-(4-((1R,5S)-3,8-diazabicyclo[3.2.1]octan-3-yl)-6,8-difluoro-2-(((2R,7aS)-2-fluorotetrahydro-1H-pyrrolizin-7a(5H)-yl)methoxy)quinazolin-7-yl)-5-ethynylnaphthalen-2-ol;
4-(4-((1R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)quinazolin-7-yl)-5-ethynyl-6-fluoronaphthalen-2-ol;
4-(4-((1R,5S)-3,8-diazabicyclo[3.2.1]octan-3-yl)-6,8-difluoro-2-(((2R,7aS)-2-fluorotetrahydro-1H-pyrrolizin-7a(5H)-yl)methoxy)quinazolin-7-yl)-5-ethyl-6-fluoronaphthalen-2-ol;
4-(4-((1S,4S)-2,5-diazabicyclo[2.2.2]octan-2-yl)-6,8-difluoro-2-(((2R,7aS)-2-fluorotetrahydro-1H-pyrrolizin-7a(5H)-yl)methoxy)quinazolin-7-yl)-5-ethyl-6-fluoronaphthalen-2-ol;
4-(4-((1S,4S)-2,5-diazabicyclo[2.2.2]octan-2-yl)-8-fluoro-2-(((2R,7aS)-2-fluorotetrahydro-1H-pyrrolizin-7a(5H)-yl)methoxy)quinazolin-7-yl)-5-ethynyl-6-fluoronaphthalen-2-ol;
4-(4-((1R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)quinazolin-7-yl)-5-ethyl-6-fluoronaphthalen-2-ol;
4-(4-((1R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)quinazolin-7-yl)-5-ethynylnaphthalen-2-ol;
4-(4-((1R,5S)-3,8-diazabicyclo[3.2.1]octan-8-yl)-8-fluoro-2-(((2R,7aS)-2-fluorotetrahydro-1H-pyrrolizin-7a(5H)-yl)methoxy)quinazolin-7-yl)-5-ethynyl-6-fluoronaphthalen-2-ol; or 4-(4-((1S,4S)-2,5-diazabicyclo[2.2.2]octan-2-yl)-6,8-difluoro-2-(((2R,7aS)-2-fluorotetrahydro-1H-pyrrolizin-7a(5H)-yl)methoxy)quinazolin-7-yl)-5-ethynyl-6-fluoronaphthalen-2-ol.

The foregoing merely summarizes certain aspects of this disclosure and is not intended, nor should it be construed, as limiting the disclosure in any way.

### Formulation, and Route of Administration

While it may be possible to administer a compound disclosed herein alone in the uses described, the compound administered normally will be present as an active ingredient in a pharmaceutical composition. Thus, in one embodiment, provided herein is a pharmaceutical composition comprising a compound disclosed herein in combination with one or more pharmaceutically acceptable excipients, such as diluents, carriers, adjuvants and the like, and, if desired, other active ingredients. See, e.g., Remington: The Science and Practice of Pharmacy, Volume I and Volume II, twenty-second edition, edited by Loyd V. Allen Jr., Philadelphia, PA, Pharmaceutical Press, 2012; Pharmaceutical Dosage Forms (Vol. 1-3), Liberman et al., Eds., Marcel Dekker, New York, NY, 1992; Handbook of Pharmaceutical Excipients (3rd Ed.), edited by Arthur H. Kibbe, American Pharmaceutical Association, Washington, 2000; Pharmaceutical Formulation: The Science and Technology of Dosage Forms (Drug Discovery), first edition, edited by GD Tovey, Royal Society of Chemistry, 2018. In one embodiment, a pharmaceutical composition comprises a therapeutically effective amount of a compound disclosed herein.

The compound(s) disclosed herein may be administered by any suitable route in the form of a pharmaceutical composition adapted to such a route and in a dose effective for the treatment intended. The compounds and compositions presented herein may, for example, be administered orally, mucosally, topically, transdermally, rectally, pulmonarily, parentally, intranasally, intravascularly, intravenously, intraarterial, intraperitoneally, intrathecally, subcutaneously, sublingually, intramuscularly, intrasternally, vaginally or by infusion techniques, in dosage unit formulations containing conventional pharmaceutically acceptable excipients.

The pharmaceutical composition may be in the form of, for example, a tablet, chewable tablet, minitablet, caplet, pill, bead, hard capsule, soft capsule, gelatin capsule, granule, powder, lozenge, patch, cream, gel, sachet, microneedle array, syrup, flavored syrup, juice, drop, injectable solution, emulsion, microemulsion, ointment, aerosol, aqueous suspension, or oily suspension. The pharmaceutical composition is typically made in the form of a dosage unit containing a particular amount of the active ingredient.

Provided herein as embodiment 270 is a pharmaceutical composition comprising the compound according to any one of embodiments 1-269, or a tautomer thereof, or a pharmaceutically acceptable salt of said compound or said tautomer, and a pharmaceutically acceptable excipient.

Provided herein as embodiment 271 is a compound according to any one of Embodiments 1-269, or a tautomer thereof, or a pharmaceutically acceptable salt of said compound or said tautomer, or the pharmaceutical composition according to embodiment 270 for use as a medicament.

### Methods of Use

As discussed herein (see, section entitled “Definitions”), the compounds described herein are to be understood to include all stereoisomers, tautomers, or pharmaceutically acceptable salts of any of the foregoing or solvates of any of the foregoing. Accordingly, the scope of the methods and uses provided in the instant disclosure is to be understood to encompass also methods and uses employing all such forms.

Besides being useful for human treatment, the compounds provided herein may be useful for veterinary treatment of companion animals, exotic animals and farm animals, including mammals, rodents, and the like. For example, animals including horses, dogs, and cats may be treated with compounds provided herein.

In one embodiment, the disclosure provides methods of using the compounds or pharmaceutical compositions of the present disclosure to treat disease conditions, including but not limited to conditions implicated by KRAS G12D mutation (e.g., cancer). The cancer types are non-small cell lung cancer, colorectal cancer, pancreatic cancer, appendiceal cancer, endometrial cancer, esophageal cancer, cancer of unknown primary, ampullary cancer, gastric cancer, small bowel cancer, sinonasal cancer, bile duct cancer, or melanoma.

KRAS G12D mutations occur with the alteration frequencies shown in the table below (TCGA data sets;¹⁻³ For example, the table shows that 32.4% of subjects with pancreatic cancer have a cancer wherein one or more cells express KRAS G12D mutant protein. Accordingly, the compounds provided herein, which bind to KRAS^{G12D} (see Section entitled “Biological Evaluation” below) are useful for treatment of subjects having a cancer, including, but not limited to the cancers listed in the table below.

| | Alteration |
|---|---|
| Cancer Type | Frequency |
| Pancreatic Adenocarcinoma (PAAD) | 32.4 |
| Colon Adenocarcinoma (COAD) | 12.25 |
| Rectal adenocarcinoma (READ) | 8.03 |
| Uterine corpus endometrial carcinoma | 6.04 |
| (UCEC) | |
| Lung Adenocarcinoma (LUAD) | 3.53 |
| Plasma Cell Tumors | 2.92 |
| Stomach Adenocarcinoma (STAD) | 2.27 |
| Bladder urothelial carcinoma (BLCA) | 1.46 |
| Cervical Squamous carcinoma (CESC) | 1.38 |
| Kidney Adenocarcinoma | 1.07 |
| Thymic Cancer | 0.81 |
| Myeloid Leukemia (LAML) | 0.69 |
| Liver Hepatocellular Carcinoma (LIHC) | 0.55 |
| Glioblastoma multiforme (GBM) | 0.51 |
| Skin Cutaneous Melanoma (SKCM) | 0.43 |
| Bladder Cancer | 0.4 |
| Prostate Adenocarcinoma (PRAD) | 0.2 |
| Breast Invasive Carcinoma (BRCA) | 0.1 |

Provided herein as embodiment 272 is a compound according to any one of embodiments 1-269 or a pharmaceutically acceptable salt thereof, or the pharmaceutical composition according to embodiment 270 for use in treating cancer.

Provided herein as Embodiment 273 is a compound according to any one of Embodiments 1-269 or a pharmaceutically acceptable salt thereof, or the pharmaceutical composition according to Embodiment 270 for use in treating cancer, wherein one or more cells express KRAS G12D mutant protein.

Provided herein as Embodiment 274 is the compound or pharmaceutical composition for use of Embodiment 272 or 273, wherein the cancer is pancreatic cancer, colorectal cancer, non-small cell lung cancer, small bowel cancer, appendiceal cancer, cancer of unknown primary, endometrial cancer, mixed cancer types, hepatobiliary cancer, small cell lung cancer, cervical cancer, germ cell cancer, ovarian cancer, gastrointestinal neuroendocrine cancer, bladder cancer, myelodysplastic/myeloproliferative neoplasms, head and neck cancer, esophagogastric cancer, soft tissue sarcoma, mesothelioma, thyroid cancer, leukemia, or melanoma.

Provided herein as Embodiment 275 is a use of the compound according to any one of Embodiments 1-269 or a pharmaceutically acceptable salt thereof, or the pharmaceutical composition according to Embodiment 270 in the preparation of a medicament for treating cancer.

Provided herein as Embodiment 276 is a use of the compound according to any one of Embodiments 1-269 or a pharmaceutically acceptable salt thereof, or the pharmaceutical composition according to Embodiment 270 in the preparation of a medicament for treating cancer, wherein one or more cells express KRAS G12D mutant protein.

Provided herein as Embodiment 277 is the use according to Embodiment 275 or 276, wherein the cancer is non-small cell lung cancer, small bowel cancer, appendiceal cancer, colorectal cancer, cancer of unknown primary, endometrial cancer, mixed cancer types, pancreatic cancer, hepatobiliary cancer, small cell lung cancer, cervical cancer, germ cell cancer, ovarian cancer, gastrointestinal neuroendocrine cancer, bladder cancer, myelodysplastic/myeloproliferative neoplasms, head and neck cancer, esophagogastric cancer, soft tissue sarcoma, mesothelioma, thyroid cancer, leukemia, or melanoma.

Provided herein as Embodiment 278 is a method of treating cancer in a subject in need thereof, the method comprising administering to the subject a therapeutically effective amount of the compound according to any one of to any one of Embodiments 1-269 or a pharmaceutically acceptable salt thereof.

Provided herein as Embodiment 279 is a method of treating cancer in a subject in need thereof, the method comprising administering to the subject a therapeutically effective amount of the compound according to any one of to any one of Embodiments 1-269 or a pharmaceutically acceptable salt thereof, wherein one or more cells express KRAS G12D mutant protein.

Provided herein as Embodiment 280 is the method according to Embodiment 278 or 279, wherein the cancer is non-small cell lung cancer, small bowel cancer, appendiceal cancer, colorectal cancer, cancer of unknown primary, endometrial cancer, mixed cancer types, pancreatic cancer, hepatobiliary cancer, small cell lung cancer, cervical cancer, germ cell cancer, ovarian cancer, gastrointestinal neuroendocrine cancer, bladder cancer, myelodysplastic/myeloproliferative neoplasms, head and neck cancer, esophagogastric cancer, soft tissue sarcoma, mesothelioma, thyroid cancer, leukemia, or melanoma.

Provided herein as Embodiment 281 is the method according to Embodiment 278 or 279, wherein the cancer is non-small cell lung cancer, colorectal cancer, pancreatic cancer, appendiceal cancer, endometrial cancer, esophageal cancer, cancer of unknown primary, ampullary cancer, gastric cancer, small bowel cancer, sinonasal cancer, bile duct cancer, or melanoma.

Provided herein as Embodiment 282 is the method according to Embodiment 281, wherein the cancer is non-small cell lung cancer.

Provided herein as Embodiment 283 is the method according to Embodiment 281, wherein the cancer is colorectal cancer.

Provided herein as Embodiment 284 is the method according to Embodiment 281, wherein the cancer is pancreatic cancer.

Provided herein as Embodiment 285 is the method according to anyone of Embodiments 278-284, wherein the subject has a cancer that was determined to have one or more cells expressing the KRAS G12D mutant protein prior to administration of the compound or a pharmaceutically acceptable salt thereof.

### Combination Therapy

The present disclosure also provides methods for combination therapies in which an agent known to modulate other pathways, or other components of the same pathway, or even overlapping sets of target enzymes are used in combination with a compound of the present disclosure or a pharmaceutically acceptable salt thereof. In one aspect, such therapy includes but is not limited to the combination of one or more compounds of the disclosure with chemotherapeutic agents, therapeutic antibodies, and radiation treatment, to provide a synergistic or additive therapeutic effect. See. e.g., U.S. Pat. No. 10,519,146 B2, issued Dec. 31, 2019; specifically, the sections from column 201 (line 37) to column 212 (line 46) and column 219 (line 64) to column 220 (line 39), which are herewith incorporated by reference.

Provided herein as Embodiment 286 is the method according to anyone of Embodiments 278-285, which further comprises simultaneous, separate, or sequential administration of an effective amount of a second compound, wherein the second compound is an Aurora kinase A inhibitor, AKT inhibitor, arginase inhibitor, CDK4/6 inhibitor, ErbB family inhibitor, ERK inhibitor, FAK inhibitor, FGFR inhibitor, Glutaminase inhibitor, IGF-1R inhibitor, KIF18A inhibitor, MCL-1 inhibitor, MEK inhibitor, mTOR inhibitor, PD-1 inhibitor, PD-L1 inhibitor, PI3K inhibitor, Raf kinase inhibitor, SHP2 inhibitor, SOS1 inhibitor, Src kinase inhibitor, or one or more chemotherapeutic agent.

In one embodiment, the second compound is administered as a pharmaceutically acceptable salt. In another embodiment the second compound is administered as a pharmaceutical composition comprising the second compound or a pharmaceutically acceptable salt thereof and a pharmaceutically acceptable excipient.

### Aurora Kinase A Inhibitors

Provided herein is the method according to anyone of Embodiments 278-285, which further comprises simultaneous, separate, or sequential administration of an effective amount of a second compound, wherein the second compound is an Aurora kinase A inhibitor.

Exemplary Aurora kinase A inhibitors for use in the methods provided herein include, but are not limited to, alisertib, cenisertib, danusertib, tozasertib, LY3295668 ((2R,4R)-1-[(3-chloro-2-fluorophenyl)methyl]-4-[[3-fluoro-6-[(5-methyl-1H-pyrazol-3-yl)amino]pyridin-2-yl]methyl]-2-methylpiperidine-4-carboxylic acid), ENMD-2076 (6-(4-methylpiperazin-1-yl)-N-(5-methyl-1H-pyrazol-3-yl)-2-[(E)-2-phenylethenyl]pyrimidin-4-amine), TAK-901 (5-(3-ethylsulfonylphenyl)-3,8-dimethyl-N-(1-methylpiperidin-4-yl)-9H-pyrido[2,3-b]indole-7-carboxamide), TT-00420 (4-[9-(2-chlorophenyl)-6-methyl-2,4,5,8,12-pentazatricyclo[8.4.0.03,7]tetradeca-1(14), 3,6,8, 10,12-hexaen-13-yl]morpholine), AMG 900 (N-[4-[3-(2-aminopyrimidin-4-yl)pyridin-2-yl]oxyphenyl]-4-(4-methylthiophen-2-yl)phthalazin-1-amine), MLN8054 (4-[[9-chloro-7-(2,6-difluorophenyl)-5H-pyrimido[5,4-d][2]benzazepin-2-yl]amino]benzoic acid), PF-03814735 (N-[2-[(1R,8S)-4-[[4-(cyclobutylamino)-5-(trifluoromethyl)pyrimidin-2-yl]amino]-11-azatricyclo[6.2.1.02,7]undeca-2(7),3,5-trien-11-yl]-2-oxoethyl]acetamide), SNS-314 (1-(3-chlorophenyl)-3-[5-[2-(thieno[3,2-d]pyrimidin-4-ylamino)ethyl]-1,3-thiazol-2-yl]urea), CYC116 (4-methyl-5-[2-(4-morpholin-4-ylanilino)pyrimidin-4-yl]-1,3-thiazol-2-amine), TAS-119, BI 811283, and TTP607.

### AKT Inhibitors

Provided herein is the method according to anyone of Embodiments 278-285, which further comprises simultaneous, separate, or sequential administration of an effective amount of a second compound, wherein the second compound is an AKT inhibitor.

Exemplary AKT inhibitors for use in the methods provided herein include, but are not limited to, afuresertib, capivasertib, ipatasertib, uprosertib, BAY1125976 (2-[4-(1-aminocyclobutyl)phenyl]-3-phenylimidazo[1,2-b]pyridazine-6-carboxamide), ARQ 092 (3-[3-[4-(1-aminocyclobutyl)phenyl]-5-phenylimidazo[4,5-b]pyridin-2-yl]pyridin-2-amine), MK2206 (8-[4-(1-aminocyclobutyl)phenyl]-9-phenyl-2H-[1,2,4]triazolo[3,4-f][1,6]naphthyridin-3-one), SR13668 (indolo[2,3-b]carbazole-2,10-dicarboxylic acid, 5,7-dihydro-6-methoxy-, 2,10-diethyl ester), ONC201 (11-benzyl-7-[(2-methylphenyl)methyl]-2,5,7,11-tetrazatricyclo[7.4.0.02,6]trideca-1(9), 5-dien-8-one), ARQ 751 (N-(3-aminopropyl)-N-[(1R)-1-(3-anilino-7-chloro-4-oxoquinazolin-2-yl)but-3-ynyl]-3-chloro-2-fluorobenzamide), RX-0201, and LY2780301.

### Arginase Inhibitors

Provided herein is the method according to anyone of Embodiments 278-285, which further comprises simultaneous, separate, or sequential administration of an effective amount of a second compound, wherein the second compound is an arginase inhibitor.

Exemplary arginase inhibitors for use in the methods provided herein include, but are not limited to, numidargistat and CB 280.

### CDK4/6 Inhibitors

Provided herein is the method according to anyone of Embodiments 278-285, which further comprises simultaneous, separate, or sequential administration of an effective amount of a second compound, wherein the second compound is a CDK4/6 inhibitor.

The term “CDK4/6” as used herein refers to cyclin dependent kinases (“CDK”) 4 and 6, which are members of the mammalian serine/threonine protein kinases.

The term “CDK4/6 inhibitor” as used herein refers to a compound that is capable of negatively modulating or inhibiting all or a portion of the enzymatic activity of CDK4 and/or 6.

Exemplary CDK4/6 inhibitors for use in the methods provided herein include, but are not limited to, abemaciclib, palbociclib, ribociclib, trilaciclib, and PF-06873600 ((pyrido[2,3-d]pyrimidin-7(8H)-one, 6-(difluoromethyl)-8-[(1R,2R)-2-hydroxy-2-methylcyclopentyl]-2-[[1-(methylsulfonyl)-4-piperidinyl]amino]).

In one embodiment, the CDK4/6 inhibitor is palbociclib.

### ErbB Family Inhibitors

Provided herein is the method according to anyone of Embodiments 278-285, which further comprises simultaneous, separate, or sequential administration of an effective amount of a second compound, wherein the second compound is an ErbB family inhibitor.

The term “ErbB family” as used herein refers to a member of a mammalian transmembrane protein tyrosine kinase family including: ErbB1 (EGFR HER1), ErbB2 (HER2), ErbB3 (HER3), and ErbB4 (HER4).

The term “ErbB family inhibitor” as used herein refers to an agent, e.g., a compound or antibody, that is capable of negatively modulating or inhibiting all or a portion of the activity of at least one member of the ErbB family. The modulation or inhibition of one or more ErbB tyrosine kinase may occur through modulating or inhibiting kinase enzymatic activity of one or more ErbB family member or by blocking homodimerization or heterodimerization of ErbB family members.

In one embodiment, the ErbB family inhibitor is an EGFR inhibitor, e.g., an anti-EGFR antibody. Exemplary anti-EGFR antibodies for use in the methods provided herein include, but are not limited to, zalutumumab, nimotuzumab, matuzumab, necitumumab, panitumumab, and cetuximab. In one embodiment, the anti-EGFR antibody is cetuximab. In one embodiment, the anti-EGFR antibody is panitumumab.

In another embodiment the ErbB family inhibitor is a HER2 inhibitor, e.g., an anti-HER2 antibody. Exemplary anti-HER-2 antibodies for use in the methods provided herein include, but are not limited to, pertuzumab, trastuzumab, and trastuzumab emtansine.

In yet another embodiment the ErbB family inhibitor is a HER3 inhibitor, e.g., an anti-HER3 antibody, such as HMBD-001 (Hummingbird Bioscience).

In one embodiment, the ErbB family inhibitor is a combination of an anti-EGFR antibody and anti-HER2 antibody.

In one embodiment, the ErbB family inhibitor is an irreversible inhibitor. Exemplary irreversible ErbB family inhibitors for use in the methods provided herein include, but are not limited to, afatinib, dacomitinib, canertinib, poziotinib, AV 412 ((N-[4-[(3-chloro-4-fluorophenyl)amino]-7-[3-methyl-3-(4-methyl-1-piperazinyl)-1-butyn-1-yl]-6-quinazolinyl]-2-propenamide)), PF 6274484 ((N-[4-[(3-chloro-4-fluorophenyl)amino]-7-methoxy-6-quinazolinyl]-2-propenamide), and HKI 357 ((E)-N-[4-[3-chloro-4-[(3-fluorophenyl)methoxy]anilino]-3-cyano-7-ethoxyquinolin-6-yl]-4-(dimethylamino)but-2-enamide).

In one embodiment, the irreversible ErbB family inhibitor is afatinib. In one embodiment, the irreversible ErbB family inhibitor is dacomitinib.

In one embodiment, the ErbB family inhibitor is a reversible inhibitor. Exemplary reversible ErbB family inhibitors for use in the methods provided herein include, but are not limited to erlotinib, gefitinib, sapitinib, varlitinib, tarloxotinib, TAK-285 (N-(2-(4-((3-chloro-4-(3-(trifluoromethyl)phenoxy)phenyl)amino)-5H-pyrrolo[3,2-d]pyrimidin-5-yl)ethyl)-3-hydroxy-3-methylbutanamide), AEE788 ((S)-6-(4-((4-ethylpiperazin-1-yl)methyl)phenyl)-N-(1-phenylethyl)-7H-pyrrolo[2,3-d]pyrimidin-4-amine), BMS 599626 ((3S)-3-morpholinylmethyl-[4-[[1-[(3-fluorophenyl)methyl]-1H-indazol-5-yl]amino]-5-methylpyrrolo[2,1-f][1,2,4]triazin-6-yl]-carbamate), and GW 583340 (N-[3-chloro-4-[(3-fluorophenyl)methoxy]phenyl]-6-[2-[(2-methylsulfonylethylamino)methyl]-1,3-thiazol-4-yl]quinazolin-4-amine).

In one embodiment, the reversible ErbB family inhibitor is sapitinib. In one embodiment, the reversible ErbB family inhibitor is tarloxotinib.

### ERK Inhibitors

Provided herein is the method according to anyone of Embodiments 278-285, which further comprises simultaneous, separate, or sequential administration of an effective amount of a second compound, wherein the second compound is an ERK inhibitor.

Exemplary ERK inhibitors for use in the methods provided herein include, but are not limited to, ulixertinib, ravoxertinib, CC-90003 (N-[2-[[2-[(2-methoxy-5-methylpyridin-4-yl)amino]-5-(trifluoromethyl)pyrimidin-4-yl]amino]-5-methylphenyl]prop-2-enamide), LY3214996 (6,6-dimethyl-2-[2-[(2-methylpyrazol-3-yl)amino]pyrimidin-4-yl]-5-(2-morpholin-4-ylethyl)thieno[2,3-c]pyrrol-4-one), KO-947 (1,5,6,8-tetrahydro-6-(phenylmethyl)-3-(4-pyridinyl)-7H-pyrazolo[4,3-g]quinazolin-7-one), ASTX029, LTT462, and JSI-1187.

### FAK Inhibitors

Provided herein is the method according to anyone of Embodiments 278-285, which further comprises simultaneous, separate, or sequential administration of an effective amount of a second compound, wherein the second compound is a FAK inhibitor.

Exemplary FAK inhibitors for use in the methods provided herein include, but are not limited to, GSK2256098 (2-[[5-chloro-2-[(5-methyl-2-propan-2-ylpyrazol-3-yl)amino]pyridin-4-yl]amino]-N-methoxybenzamide), PF-00562271 (N-methyl-N-[3-[[[2-[(2-oxo-1,3-dihydroindol-5-yl)amino]-5-(trifluoromethyl)pyrimidin-4-yl]amino]methyl]pyridin-2-yl]methanesulfonamide), VS-4718 (2-[[2-(2-methoxy-4-morpholin-4-ylanilino)-5-(trifluoromethyl)pyridin-4-yl]amino]-N-methylbenzamide), and APG-2449.

### FGFR Inhibitors

Provided herein is the method according to anyone of Embodiments 278-285, which further comprises simultaneous, separate, or sequential administration of an effective amount of a second compound, wherein the second compound is an FGFR inhibitor.

Exemplary FGFR inhibitors for use in the methods provided herein include, but are not limited to, futibatinib, pemigatinib, ASP5878 (2-[4-[[5-[(2,6-difluoro-3,5-dimethoxyphenyl)methoxy]pyrimidin-2-yl]amino]pyrazol-1-yl]ethanol), AZD4547 (N-[5-[2-(3,5-dimethoxyphenyl)ethyl]-1H-pyrazol-3-yl]-4-[(3S,5R)-3,5-dimethylpiperazin-1-yl]benzamide), debio 1347 ([5-amino-1-(2-methyl-3H-benzimidazol-5-yl)pyrazol-4-yl]-(1H-indol-2-yl)methanone), INCB062079, H₃B-6527 (N-[2-[[6-[(2,6-dichloro-3,5-dimethoxyphenyl)carbamoyl-methylamino]pyrimidin-4-yl]amino]-5-(4-ethylpiperazin-1-yl)phenyl]prop-2-enamide), ICP-105, CPL304110, HMPL-453, and HGS1036.

### Glutaminase Inhibitors

Provided herein is the method according to anyone of Embodiments 278-285, which further comprises simultaneous, separate, or sequential administration of an effective amount of a second compound, wherein the second compound is a glutaminase inhibitor.

Exemplary glutaminase inhibitors for use in the methods provided herein include, but are not limited to, telaglenastat, IPN60090, and OP 330.

### IGF-1R Inhibitors

Provided herein is the method according to anyone of Embodiments 278-285, which further comprises simultaneous, separate, or sequential administration of an effective amount of a second compound, wherein the second compound is an IGF-1R inhibitor.

Exemplary IGF-1R inhibitors for use in the methods provided herein include, but are not limited to, cixutumumab, dalotuzumab, linsitinib, ganitumab, robatumumab, BMS-754807 ((2S)-1-[4-[(5-cyclopropyl-1H-pyrazol-3-yl)amino]pyrrolo[2,1-f][1,2,4]triazin-2-yl]-N-(6-fluoropyridin-3-yl)-2-methylpyrrolidine-2-carboxamide), KW-2450 (N-[5-[[4-(2-hydroxyacetyl)piperazin-1-yl]methyl]-2-[(E)-2-(1H-indazol-3-yl)ethenyl]phenyl]-3-methylthiophene-2-carboxamide), PL225B, AVE1642, and BIIB022.

### KIF18A Inhibitors

Provided herein is the method according to anyone of Embodiments 278-285, which further comprises simultaneous, separate, or sequential administration of an effective amount of a second compound, wherein the second compound is a KIF18A inhibitor.

Exemplary KIF18A inhibitors for use in the methods provided herein include, but are not limited to, the inhibitors disclosed in US 2020/0239441, WO 2020/132649, WO 2020/132651, and WO 2020/132653, each of which is herewith incorporated by reference in its entirety.

### MCL-1 Inhibitors

Provided herein is the method according to anyone of Embodiments 278-285, which further comprises simultaneous, separate, or sequential administration of an effective amount of a second compound, wherein the second compound is an MCL-1 inhibitor.

Exemplary MEK inhibitors for use in the methods provided herein include, but are not limited to, murizatoclax, tapotoclax, AZD 5991 ((3aR)-5-chloro-2,11, 12, 24, 27,29-hexahydro-2,3,24,33-tetramethyl-22H-9,4,8-(metheniminomethyno)-14, 20:26,23-dimetheno-10H,20H-pyrazolo[4,3-1][2, 15, 22, 18, 19]benzoxadithiadiazacyclohexacosine-32-carboxylic acid), MIK 665 ((αR)-α-[[(5S)-5-[3-Chloro-2-methyl-4-[2-(4-methyl-1-piperazinyl)ethoxy]phenyl]-6-(4-fluorophenyl)thieno[2,3-d]pyrimidin-4-yl]oxy]-2-[[2-(2-methoxyphenyl)-4-pyrimidinyl]methoxy]benzenepropanoic acid), and ABBV-467.

In one embodiment, the MCL-1 inhibitor is murizatoclax. In another embodiment, the MCL-1 inhibitor is tapotoclax.

### MEK Inhibitors

Provided herein is the method according to anyone of Embodiments 278-285, which further comprises simultaneous, separate, or sequential administration of an effective amount of a second compound, wherein the second compound is MEK inhibitor.

Exemplary MEK inhibitors for use in the methods provided herein include, but are not limited to, trametinib, cobimetinib, selumetinib, pimasertib, refametinib, PD-325901 (N-[(2R)-2,3-dihydroxypropoxy]-3,4-difluoro-2-(2-fluoro-4-iodoanilino)benzamide), AZD8330 (2-(2-fluoro-4-iodoanilino)-N-(2-hydroxyethoxy)-1,5-dimethyl-6-oxopyridine-3-carboxamide), GDC-0623 (5-(2-fluoro-4-iodoanilino)-N-(2-hydroxyethoxy)imidazo[1,5-a]pyridine-6-carboxamide), RO4987655 (3,4-difluoro-2-(2-fluoro-4-iodoanilino)-N-(2-hydroxyethoxy)-5-[(3-oxooxazinan-2-yl)methyl]benzamide), TAK-733 (3-[(2R)-2,3-dihydroxypropyl]-6-fluoro-5-(2-fluoro-4-iodoanilino)-8-methylpyrido[2,3-d]pyrimidine-4,7-dione), PD0325901 (N-[(2R)-2,3-dihydroxypropoxy]-3,4-difluoro-2-(2-fluoro-4-iodoanilino)benzamide), CI-1040 (2-(2-chloro-4-iodophenylamino)-N-(cyclopropylmethoxy)-3,4-difluorobenzamide), PD318088 (5-bromo-N-(2,3-dihydroxypropoxy)-3,4-difluoro-2-(2-fluoro-4-iodophenylamino)benzamide), PD98059 (2-(2-amino-3-methoxyphenyl)-4H-chromen-4-one), PD334581 (N-[5-[3,4-Difluoro-2-[(2-fluoro-4-iodophenyl)amino]phenyl]-1,3,4-oxadiazol-2-yl]-4-morpholineethanamine), FCN-159, CS3006, HL-085, SHR 7390, and WX-554.

In one embodiment, the MEK inhibitor is trametinib.

mTOR Inhibitors

Provided herein is the method according to anyone of Embodiments 278-285, which further comprises simultaneous, separate, or sequential administration of an effective amount of a second compound, wherein the second compound is an mTOR inhibitor.

Exemplary mTOR inhibitors for use in the methods provided herein include, but are not limited to, everolimus, rapamycin, zotarolimus (ABT-578), ridaforolimus (deforolimus, MK-8669), sapanisertib, buparlisib, pictilisib, vistusertib, dactolisib, Torin-1 (1-(4-(4-propionylpiperazin-1-yl)-3-(trifluoromethyl)cyclohexyl)-9-(quinolin-3-yl)benzo[h][1,6]naphthyridin-2(1H)-one), GDC-0349 ((S)-1-ethyl-3-(4-(4-(3-methylmorpholino)-7-(oxetan-3-yl)-5,6,7,8-tetrahydropyrido[3,4-d]pyrimidin-2-yl)phenyl)urea), and VS-5584 (SB2343, (5-(8-methyl-2-morpholin-4-yl-9-propan-2-ylpurin-6-yl)pyrimidin-2-amine).

In one embodiment, the mTOR inhibitor is everolimus.

### PD-1 Inhibitors

Provided herein is the method according to anyone of Embodiments 278-285, which further comprises simultaneous, separate, or sequential administration of an effective amount of a second compound, wherein the second compound is a PD-1 inhibitor.

Exemplary PD-1 inhibitors for use in the methods provided herein include, but are not limited to, pembrolizumab, nivolumab, cemiplimab, spartalizumab (PDR001), camrelizumab (SHR1210), sintilimab (IBI308), tislelizumab (BGB-A317), toripalimab (JS 001), dostarlimab (TSR-042, WBP-285), INCMGA00012 (MGA012), AMP-224, AMP-514, and the anti-PD-1 antibody as described in U.S. Pat. No. 10,640,504 B2 (the “Anti-PD-1 Antibody A,” column 66, line 56 to column 67, line 24 and column 67, lines 54-57), which is incorporated herein by reference.

In one embodiment, the PD-1 inhibitor is pembrolizumab. In another embodiment the PD-1 inhibitor is the Anti-PD-1 Antibody A.

### PD-L1 Inhibitors

Provided herein is the method according to anyone of Embodiments 278-285, which further comprises simultaneous, separate, or sequential administration of an effective amount of a second compound, wherein the second compound is a PD-L1 inhibitor.

Exemplary PD-L1 inhibitors for use in the methods provided herein include, but are not limited to, atezolizumab, avelumab, durvalumab, ZKAB001, TG-1501, SHR-1316, MSB2311, MDX-1105, KN035, IMC-001, HLX20, FAZ053, CS1001, CK-301, CBT-502, BGB-A333, BCD-135, and A167.

In one embodiment, the PD-L1 inhibitor is atezolizumab.

### PI3K Inhibitors

Provided herein is the method according to anyone of Embodiments 278-285, which further comprises simultaneous, separate, or sequential administration of an effective amount of a second compound, wherein the second compound is a PI3K inhibitor.

Exemplary PI3K inhibitors for use in the methods provided herein include, but are not limited to, idelalisib, copanlisib, duvelisib, alpelisib, taselisib, perifosine, buparlisib, umbralisib, pictilisib, dactolisib, voxtalisib, sonolisib, tenalisib, serabelisib, acalisib, CUDC-907 (N-hydroxy-2-[[2-(6-methoxypyridin-3-yl)-4-morpholin-4-ylthieno[3,2-d]pyrimidin-6-yl]methyl-methylamino]pyrimidine-5-carboxamide), ME-401 (N-[2-methyl-1-[2-(1-methylpiperidin-4-yl)phenyl]propan-2-yl]-4-(2-methylsulfonylbenzimidazol-1-yl)-6-morpholin-4-yl-1,3,5-triazin-2-amine), IPI-549 (2-amino-N-[(1S)-1-[8-[2-(1-methylpyrazol-4-yl)ethynyl]-1-oxo-2-phenylisoquinolin-3-yl]ethyl]pyrazolo[1,5-a]pyrimidine-3-carboxamide), SF1126 ((2S)-2-[[(2S)-3-carboxy-2-[[2-[[(2S)-5-(diaminomethylideneamino)-2-[[4-oxo-4-[[4-(4-oxo-8-phenylchromen-2-yl)morpholin-4-ium-4-yl]methoxy]butanoyl]amino]pentanoyl]amino]acetyl]amino]propanoyl]amino]-3-hydroxypropanoate), XL147 (N-[3-(2,1,3-benzothiadiazol-5-ylamino)quinoxalin-2-yl]-4-methylbenzenesulfonamide), GSK1059615 ((5Z)-5-[(4-pyridin-4-ylquinolin-6-yl)methylidene]-1,3-thiazolidine-2,4-dione), and AMG 319 (N-[(1S)-1-(7-fluoro-2-pyridin-2-ylquinolin-3-yl)ethyl]-7H-purin-6-amine).

### Raf Kinase Inhibitors

Provided herein is the method according to anyone of Embodiments 278-285, which further comprises simultaneous, separate, or sequential administration of an effective amount of a second compound, wherein the second compound is a Raf kinase inhibitor.

The term “RAF kinase” as used herein refers to a member of a mammalian serine/threonine kinases composed of three isoforms (C-Raf, B-Raf and A-Raf) and includes homodimers of each isoform as well as heterodimers between isoforms, e.g., C-Raf/B-Raf heterodimers.

The term “Raf kinase inhibitor” as used herein refers to a compound that is capable of negatively modulating or inhibiting all or a portion of the enzymatic activity of one or more member of the Raf family kinases, or is capable of disrupting Raf homodimer or heterodimer formation to inhibit activity.

In one embodiment, the Raf kinase inhibitor includes, but is not limited to, encorafenib, sorafenib, lifirafenib, vemurafenib, dabrafenib, PLX-8394 (N-(3-(5-(2-cyclopropylpyrimidin-5-yl)-3a,7a-dihydro-1H-pyrrolo[2,3-b]pyridine-3-carbonyl)-2,4-difluorophenyl)-3-fluoropyrrolidine-1-sulfonamide), Raf-709 (N-(2-methyl-5,-morpholino-6′-((tetrahydro-2H-pyran-4-yl)oxy)-[3,3′-bipyridin]-5-yl)-3-(trifluoromethyl)benzamide), LXH₂₅₄ (N-(3-(2-(2-hydroxyethoxy)-6-morpholinopyridin-4-yl)-4-methylphenyl)-2-(trifluoromethyl)isonicotinamide), LY3009120 (1-(3,3-dimethylbutyl)-3-(2-fluoro-4-methyl-5-(7-methyl-2-(methylamino)pyrido[2,3-d]pyrimidin-6-yl)phenyl)urea), Tak-632 (N-(7-cyano-6-(4-fluoro-3-(2-(3-(trifluoromethyl)phenyl)acetamido)phenoxy)benzo[d]thiazol-2-yl)cyclopropanecarboxamide), CEP-32496 (1-(3-((6,7-dimethoxyquinazolin-4-yl)oxy)phenyl)-3-(5-(1,1,1-trifluoro-2-methylpropan-2-yl)isoxazol-3-yl)urea), CCT196969 (1-(3-(tert-butyl)-1-phenyl-1H-pyrazol-5-yl)-3-(2-fluoro-4-((3-oxo-3,4-dihydropyrido[2,3-b]pyrazin-8-yl)oxy)phenyl)urea), and RO5126766 (N-[3-fluoro-4-[[4-methyl-2-oxo-7-(2-pyrimidinyloxy)-2H-1-benzopyran-3-yl]methyl]-2-pyridinyl]-N′-methyl-sulfamide).

In one embodiment, the Raf kinase inhibitor is encorafenib. In one embodiment, the Raf kinase inhibitor is sorafenib. In one embodiment, the Raf kinase inhibitor is lifirafenib.

### SHP2 Inhibitors

Provided herein is the method according to anyone of Embodiments 278-285, which further comprises simultaneous, separate, or sequential administration of an effective amount of a second compound, wherein the second compound is a SHP2 inhibitor.

Exemplary SHP2 inhibitors for use in the methods provided herein include, but are not limited to, SHP-099 (6-(4-amino-4-methylpiperidin-1-yl)-3-(2,3-dichlorophenyl)pyrazin-2-amine dihydrochloride), RMC-4550 ([3-[(3S,4S)-4-amino-3-methyl-2-oxa-8-azaspiro[4.5]decan-8-yl]-6-(2,3-dichlorophenyl)-5-methylpyrazin-2-yl]methanol), TNO155, (3S,4S)-8-[6-amino-5-(2-amino-3-chloropyridin-4-yl)sulfanylpyrazin-2-yl]-3-methyl-2-oxa-8-azaspiro[4.5]decan-4-amine), and RMC-4630 (Revolution Medicine). In one embodiment, the SHP inhibitor for use in the methods provided herein is RMC-4630 (Revolution Medicine).

In another embodiment, exemplary SHP2 inhibitors for use in the methods provided herein include, but are not limited to, 3-[(1R,3R)-1-amino-3-methoxy-8-azaspiro[4.5]dec-8-yl]-6-(2,3-dichlorophenyl)-5-methyl-2-pyrazinemethanol (CAS 2172651-08-8), 3-[(3S,4S)-4-amino-3-methyl-2-oxa-8-azaspiro[4.5]dec-8-yl]-6-[(2,3-dichlorophenyl)thio]-5-methyl-2-pyrazinemethanol (CAS 2172652-13-8), 3-[(3S,4S)-4-amino-3-methyl-2-oxa-8-azaspiro[4.5]dec-8-yl]-6-[[3-chloro-2-(3-hydroxy-1-azetidinyl)-4-pyridinyl]thio]-5-methyl-2-pyrazinemethanol (CAS 2172652-38-7), and 6-[(2-amino-3-chloro-4-pyridinyl)thio]-3-[(3S,4S)-4-amino-3-methyl-2-oxa-8-azaspiro[4.5]dec-8-yl]-5-methyl-2-pyrazinemethanol (CAS 2172652-48-9).

In another embodiment, exemplary SHP2 inhibitors for use in the methods provided herein include, but are not limited to, 1-[5-(2,3-dichlorophenyl)-6-methylimidazo[1,5-a]pyrazin-8-yl]-4-methyl-4-piperidinamine (CAS 2240981-75-1), (1R)-8-[5-(2,3-dichlorophenyl)-6-methylimidazo[1,5-a]pyrazin-8-yl]-8-azaspiro[4.5]decan-1-amine (CAS 2240981-78-4), (3S,4S)-8-[7-(2,3-dichlorophenyl)-6-methylpyrazolo[1,5-a]pyrazin-4-yl]-3-methyl-2-oxa-8-azaspiro[4.5]decan-4-amine (CAS 2240982-45-8), (3S,4S)-8-[7-[(2-amino-3-chloro-4-pyridinyl)thio]pyrazolo[1,5-a]pyrazin-4-yl]-3-methyl-2-oxa-8-azaspiro[4.5]decan-4-amine (CAS 2240982-57-2), 4-[(3S,4S)-4-amino-3-methyl-2-oxa-8-azaspiro[4.5]dec-8-yl]-7-(2,3-dichlorophenyl)-6-methyl-pyrazolo[1,5-a]pyrazine-2-methanol (CAS 2240982-69-6), 7-[(2-amino-3-chloro-4-pyridinyl)thio]-4-[(3S,4S)-4-amino-3-methyl-2-oxa-8-azaspiro[4.5]dec-8-yl]-6-methyl-pyrazolo[1,5-a]pyrazine-2-methanol (CAS 2240982-73-2), and (3S,4S)-8-[7-[(2-amino-3-chloro-4-pyridinyl)thio]-6-methylpyrazolo[1,5-a]pyrazin-4-yl]-3-methyl-2-oxa-8-azaspiro[4.5]decan-4-amine (CAS 2240982-77-6).

In one embodiment, the SHP inhibitor for use in the methods provided herein is (1R)-8-[5-(2,3-dichlorophenyl)-6-methylimidazo[1,5-a]pyrazin-8-yl]-8-azaspiro[4.5]decan-1-amine (CAS 2240981-78-4).

In another embodiment, exemplary SHP2 inhibitors for use in the methods provided herein include, but are not limited to 3-[(1R)-1-amino-8-azaspiro[4.5]dec-8-yl]-6-(2,3-dichlorophenyl)-5-hydroxy-2-pyridinemethanol (CAS 2238840-54-3), 3-[(1R)-1-amino-8-azaspiro[4.5]dec-8-yl]-6-[(2,3-dichlorophenyl)thio]-5-hydroxy-2-pyridinemethanol (CAS 2238840-56-5), 5-[(1R)-1-amino-8-azaspiro[4.5]dec-8-yl]-2-(2,3-dichlorophenyl)-3-pyridinol (CAS 2238840-58-7), 3-[(1R)-1-amino-8-azaspiro[4.5]dec-8-yl]-6-(2,3-dichlorophenyl)-5-methyl-2-pyridinemethanol (CAS 2238840-60-1), (1R)-8-[6-(2,3-dichlorophenyl)-5-methyl-3-pyridinyl]-8-azaspiro[4.5]decan-1-amine (CAS 2238840-62-3), 3-[(1R)-1-amino-8-azaspiro[4.5]dec-8-yl]-6-[(2,3-dichlorophenyl)thio]-5-methyl-2-pyridinemethanol (CAS 2238840-63-4), (1R)-8-[6-[(2,3-dichlorophenyl)thio]-5-methyl-3-pyridinyl]-8-azaspiro[4.5]decan-1-amine (CAS 2238840-64-5), 5-(4-amino-4-methyl-1-piperidinyl)-2-[(2,3-dichlorophenyl)thio]-3-pyridinol (CAS 2238840-65-6), 5-[(1R)-1-amino-8-azaspiro[4.5]dec-8-yl]-2-[(2,3-dichlorophenyl)thio]-3-pyridinol (CAS 2238840-66-7), 6-[(2-amino-3-chloro-4-pyridinyl)thio]-3-[(3S,4S)-4-amino-3-methyl-2-oxa-8-azaspiro[4.5]dec-8-yl]-5-hydroxy-2-pyridinemethanol (CAS 2238840-67-8), 3-(4-amino-4-methyl-1-piperidinyl)-6-(2,3-dichlorophenyl)-5-hydroxy-2-pyridinemethanol (CAS 2238840-68-9), 3-[(3S,4S)-4-amino-3-methyl-2-oxa-8-azaspiro[4.5]dec-8-yl]-6-(2,3-dichlorophenyl)-5-methyl-2-pyridinemethanol (CAS 2238840-69-0), 6-[(2-amino-3-chloro-4-pyridinyl)thio]-3-[(3S,4S)-4-amino-3-methyl-2-oxa-8-azaspiro[4.5]dec-8-yl]-5-methyl-2-pyridinemethanol (CAS 2238840-70-3), 3-(4-amino-4-methyl-1-piperidinyl)-6-(2,3-dichlorophenyl)-5-methyl-2-pyridinemethanol (CAS 2238840-71-4), 6-[(2-amino-3-chloro-4-pyridinyl)thio]-3-(4-amino-4-methyl-1-piperidinyl)-2-pyridinemethanol (CAS 2238840-72-5), 5-[(2-amino-3-chloro-4-pyridinyl)thio]-2-[(3S,4S)-4-amino-3-methyl-2-oxa-8-azaspiro[4.5]dec-8-yl]-6-methyl-3-pyridinemethanol (CAS 2238840-73-6), 2-[(3S,4S)-4-amino-3-methyl-2-oxa-8-azaspiro[4.5]dec-8-yl]-5-(2,3-dichlorophenyl)-6-methyl-3-pyridinemethanol (CAS 2238840-74-7), 3-[(3S,4S)-4-amino-3-methyl-2-oxa-8-azaspiro[4.5]dec-8-yl]-6-(2,3-dichlorophenyl)-5-hydroxy-2-pyridinemethanol (CAS 2238840-75-8), and 2-[(2-amino-3-chloro-4-pyridyl)sulfanyl]-5-[(3S,4S)-4-amino-3-methyl-2-oxa-8-azaspiro[4.5]decan-8-yl]-6-(hydroxymethyl)pyridin-3-ol.

In one embodiment, the SHP inhibitor for use in the methods provided herein is 3-[(1R)-1-amino-8-azaspiro[4.5]dec-8-yl]-6-[(2,3-dichlorophenyl)thio]-5-hydroxy-2-pyridinemethanol (CAS 2238840-56-5).

In one embodiment, the SHP2 inhibitor for use in the methods provided herein is an inhibitor disclosed in U.S. Pat. No. 10,590,090 B2, US 2020/017517 A1, US 2020/017511 A1, or WO 2019/075265 A1, each of which is herewith incorporated by reference in its entirety.

### SOS1 Inhibitors

Provided herein is the method according to anyone of Embodiments 278-285, which further comprises simultaneous, separate, or sequential administration of an effective amount of a second compound, wherein the second compound is an SOS1 inhibitor.

Exemplary SOS1 inhibitors for use in the methods provided herein include, but are not limited to, BI 3406 (N-[(1R)-1-[3-amino-5-(trifluoromethyl)phenyl]ethyl]-7-methoxy-2-methyl-6-[(3S)-oxolan-3-yl]oxyquinazolin-4-amine), and BI 1701963.

### Src Kinase Inhibitors

Provided herein is the method according to anyone of Embodiments 278-285, which further comprises simultaneous, separate, or sequential administration of an effective amount of a second compound, wherein the second compound is a Src kinase inhibitor.

The term “Src kinase” as used herein refers to a member of a mammalian nonreceptor tyrosine kinase family including: Src, Yes, Fyn, and Fgr (SrcA subfamily); Lck, Hck, Blk, and Lyn (SrcB subfamily), and Frk subfamily.

The term “Src kinase inhibitor” as used herein refers to a compound that is capable of negatively modulating or inhibiting all or a portion of the enzymatic activity of one or more member of the Src kinases.

Exemplary Src kinase inhibitors for use in the methods provided herein include, but are not limited to, dasatinib, ponatinib, vandetanib, bosutinib, saracatinib, KX2-391 (N-benzyl-2-(5-(4-(2-morpholinoethoxy)phenyl)pyridin-2-yl)acetamide), SU6656 ((Z)-N,N-dimethyl-2-oxo-3-((4,5,6,7-tetrahydro-1H-indol-2-yl)methylene)indoline-5-sulfonamide), PP 1 (1-(tert-butyl)-3-(p-tolyl)-1H-pyrazolo[3,4-d]pyrimidin-4-amine), WH-4-023 (2,6-dimethylphenyl(2,4-dimethoxyphenyl)(2-((4-(4-methylpiperazin-1-yl)phenyl)amino)pyrimidin-4-yl)carbamate), and KX-01 (N-benzyl-2-(5-(4-(2-morpholinoethoxy)phenyl)pyridin-2-yl)acetamide).

In one embodiment, the Src kinase inhibitor is dasatinib. In one embodiment, the Src kinase inhibitor is saracatinib. In one embodiment, the Src kinase inhibitor is ponatinib. In one embodiment, the Src kinase inhibitor is vandetanib. In one embodiment, the Src kinase inhibitor is KX-01.

### Chemotherapeutic Aments

Provided herein is the method according to anyone of Embodiments 278-285, which further comprises simultaneous, separate, or sequential administration of an effective amount of a second compound, wherein the second compound is one or more chemotherapeutic agent.

Exemplary chemotherapeutic agents for use in the methods provided herein include, but are not limited to, leucovorin calcium (calcium folinate), 5-fluorouracil, irinotecan, oxaliplatin, cisplatin, carboplatin, pemetrexed, docetaxel, paclitaxel, gemcitabine, vinorelbine, chlorambucil, cyclophosphamide, and methotrexate.

### Definitions

The following definitions are provided to assist in understanding the scope of this disclosure.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the standard deviation found in their respective testing measurements.

As used herein, if any variable occurs more than one time in a chemical formula, its definition on each occurrence is independent of its definition at every other occurrence. If the chemical structure and chemical name conflict, the chemical structure is determinative of the identity of the compound.

### Stereoisomers

The compounds of the present disclosure may contain, for example, double bonds, one or more asymmetric carbon atoms, and bonds with a hindered rotation, and therefore, may exist as stereoisomers, such as double-bond isomers (i.e., geometric isomers (E/Z)), enantiomers, diastereomers, and atropoisomers. Accordingly, the scope of the instant disclosure is to be understood to encompass all possible stereoisomers of the illustrated compounds, including the stereoisomerically pure form (for example, geometrically pure, enantiomerically pure, diastereomerically pure, and atropoisomerically pure) and stereoisomeric mixtures (for example, mixtures of geometric isomers, enantiomers, diastereomers, and atropoisomers, or mixture of any of the foregoing) of any chemical structures disclosed herein (in whole or in part), unless the stereochemistry is specifically identified.

If the stereochemistry of a structure or a portion of a structure is not indicated with, for example, bold or dashed lines, the structure or portion of the structure is to be interpreted as encompassing all stereoisomers of it. If the stereochemistry of a structure or a portion of a structure is indicated with, for example, bold or dashed lines, the structure or portion of the structure is to be interpreted as encompassing only the stereoisomer indicated, unless otherwise noted.

For example,

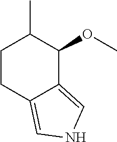

represents

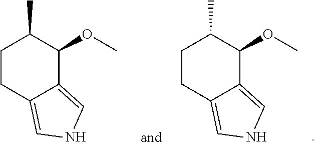

Similarly, for example, the chemical name (4R)-4-methoxy-5-methyl-4,5,6,7-tetrahydro-2H-isoindole represents (4R,5R)-4-methoxy-5-methyl-4,5,6,7-tetrahydro-2H-isoindole and (4R,5S)-4-methoxy-5-methyl-4,5,6,7-tetrahydro-2H-isoindole.

As a further example,

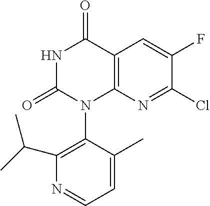 [SMILES: Cc1ccnc(C(C)C)c1-n1c(=O)[nH]c(=O)c2cc(F)c(Cl)nc21]

represents

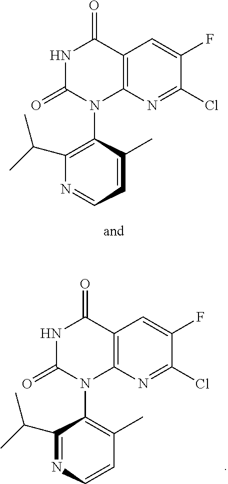

Similarly, for example, the chemical name 7-chloro-6-fluoro-1-(2-isopropyl-4-methylpyridin-3-yl)pyrido[2,3-d]pyrimidine-2,4(1H,3H)-dione represents (M)-7-chloro-6-fluoro-1-(2-isopropyl-4-methylpyridin-3-yl)pyrido[2,3-d]pyrimidine-2,4(1H,3H)-dione and (P)-7-chloro-6-fluoro-1-(2-isopropyl-4-methylpyridin-3-yl)pyrido[2,3-d]pyrimidine-2,4(1H,3H)-dione.

In certain instances, a bond drawn with a wavy line indicates that both stereoisomers are encompassed. This is not to be confused with a wavy line drawn perpendicular to a bond which indicates the point of attachment of a group to the rest of the molecule.

The term “stereoisomer” or “stereoisomerically pure” compound as used herein refers to one stereoisomer (for example, geometric isomer, enantiomer, diastereomer and atropoisomer) of a compound that is substantially free of other stereoisomers of that compound. For example, a stereoisomerically pure compound having one chiral center will be substantially free of the mirror image enantiomer of the compound and a stereoisomerically pure compound having two chiral centers will be substantially free of other enantiomers or diastereomers of the compound. A typical stereoisomerically pure compound comprises greater than about 80% by weight of one stereoisomer of the compound and equal or less than about 20% by weight of other stereoisomers of the compound, greater than about 90% by weight of one stereoisomer of the compound and equal or less than about 10% by weight of the other stereoisomers of the compound, greater than about 95% by weight of one stereoisomer of the compound and equal or less than about 5% by weight of the other stereoisomers of the compound, or greater than about 97% by weight of one stereoisomer of the compound and equal or less than about 3% by weight of the other stereoisomers of the compound.

This disclosure also encompasses the pharmaceutical compositions comprising stereoisomerically pure forms and the use of stereoisomerically pure forms of any compounds disclosed herein. Further, this disclosure also encompasses pharmaceutical compositions comprising mixtures of stereoisomers of any compounds disclosed herein and the use of said pharmaceutical compositions or mixtures of stereoisomers. These stereoisomers or mixtures thereof may be synthesized in accordance with methods well known in the art and methods disclosed herein. Mixtures of stereoisomers may be resolved using standard techniques, such as chiral columns or chiral resolving agents. Further, this disclosure encompasses pharmaceutical compositions comprising mixtures of any of the compounds disclosed herein and one or more other active agents disclosed herein. See, for example, Jacques et al., Enantiomers, Racemates and Resolutions (Wiley-Interscience, New York, 1981); Wilen et al., Tetrahedron 33:2725; Eliel, Stereochemistry of Carbon Compounds (McGraw-Hill, NY, 1962); and Wilen, Tables of Resolving Agents and Optical Resolutions, page 268 (Eliel, Ed., Univ. of Notre Dame Press, Notre Dame, IN, 1972).

### Tautomers

As known by those skilled in the art, certain compounds disclosed herein may exist in one or more tautomeric forms. Because one chemical structure may only be used to represent one tautomeric form, it will be understood that for convenience, referral to a compound of a given structural formula includes other tautomers of said structural formula. Accordingly, the scope of the instant disclosure is to be understood to encompass all tautomeric forms of the compounds disclosed herein.

### Isotopically-Labelled Compounds

Further, the scope of the present disclosure includes all pharmaceutically acceptable isotopically-labelled compounds of the compounds disclosed herein, such as the compounds of Formula I, wherein one or more atoms are replaced by atoms having the same atomic number, but an atomic mass or mass number different from the atomic mass or mass number usually found in nature. Examples of isotopes suitable for inclusion in the compounds disclosed herein include isotopes of hydrogen, such as ²H and ³H, carbon, such as ¹¹C, ¹³C and ¹⁴C, chlorine, such as ³⁶CI, fluorine, such as ¹⁸F, iodine, such as ¹²³I and ¹²⁵I, nitrogen, such as ¹³N and ¹⁵N, oxygen, such as ¹⁵O, ¹⁷ and ¹⁸O, phosphorus, such as ³²P, and sulphur, such as ³⁵S. Certain isotopically-labelled compounds of Formula I, for example, those incorporating a radioactive isotope, are useful in drug and/or substrate tissue distribution studies. The radioactive isotopes tritium (³H) and carbon-14 (¹⁴C) are particularly useful for this purpose in view of their ease of incorporation and ready means of detection. Substitution with isotopes such as deuterium (²H or D) may afford certain therapeutic advantages resulting from greater metabolic stability, for example, increased in vivo half-life or reduced dosage requirements, and hence may be advantageous in some circumstances. Substitution with positron emitting isotopes, such as ¹¹C, ¹⁸F, ¹⁵O and ¹³N, can be useful in Positron Emission Topography (PET) studies, for example, for examining target occupancy. Isotopically-labelled compounds of the compounds disclosed herein can generally be prepared by conventional techniques known to those skilled in the art or by processes analogous to those described in the accompanying General Synthetic Schemes and Examples using an appropriate isotopically-labelled reagent in place of the non-labelled reagent previously employed.

### Solvates

As discussed above, the compounds disclosed herein and the stereoisomers, tautomers, and isotopically-labelled forms thereof or a pharmaceutically acceptable salt of any of the foregoing may exist in solvated or unsolvated forms.

The term “solvate” as used herein refers to a molecular complex comprising a compound or a pharmaceutically acceptable salt thereof as described herein and a stoichiometric or non-stoichiometric amount of one or more pharmaceutically acceptable solvent molecules. If the solvent is water, the solvate is referred to as a “hydrate.”

Accordingly, the scope of the instant disclosure is to be understood to encompass all solvents of the compounds disclosed herein and the stereoisomers, tautomers and isotopically-labelled forms thereof or a pharmaceutically acceptable salt of any of the foregoing.

### Miscellaneous Definitions

This section will define additional terms used to describe the scope of the compounds, compositions and uses disclosed herein.

The term “aryl” refers to an aromatic hydrocarbon group having 6-20 carbon atoms in the ring portion. Typically, aryl is monocyclic, bicyclic or tricyclic aryl having 6-20 carbon atoms. Furthermore, the term “aryl” as used herein, refers to an aromatic substituent which can be a single aromatic ring, or multiple aromatic rings that are fused together. Non-limiting examples include phenyl, naphthyl or tetrahydronaphthyl, each of which may optionally be substituted with 1-4 substituents, such as alkyl, trifluoromethyl, cycloalkyl, halogen, hydroxy, alkoxy, acyl, alkyl-C(O)—O—, aryl-O—, heteroaryl-O—, amino, thiol, alkyl-S—, aryl-S-nitro, cyano, carboxy, alkyl-O—C(O)—, carbamoyl, alkyl-S(O)—, sulfonyl, sulfonamido, phenyl, and heterocyclyl.

The terms “C₁₋₄ alkyl,” and “C₁₋₆alkyl” as used herein refer to a straight or branched chain hydrocarbon containing from 1 to 4, and 1 to 6 carbon atoms, respectively. Representative examples of C₁₋₄ alkyl or C₁₋₆ alkyl include, but are not limited to, methyl, ethyl, n-propyl, iso-propyl, n-butyl, sec-butyl, iso-butyl, tert-butyl, pentyl and hexyl.

The terms “C₁₋₄ alkylene” and “C₁₋₆ alkylene” refer to a straight or branched divalent alkyl group as defined herein containing 1 to 4, and 1 to 6 carbon atoms, respectively. Representative examples of alkylene include, but are not limited to, methylene, ethylene, n-propylene, iso-propylene, n-butylene, sec-butylene, iso-butylene, tert-butylene, n-pentylene, isopentylene, neopentylene, n-hexylene and the like.

The term “C₂₋₄ alkenyl” as used herein refers to a saturated hydrocarbon containing 2 to 4 carbon atoms having at least one carbon-carbon double bond. Alkenyl groups include both straight and branched moieties. Representative examples of C₂₋₄ alkenyl include, but are not limited to, 1-propenyl, 2-propenyl, 2-methyl-2-propenyl, and butenyl.

The term “C₂₋₄ alkynyl” as used herein refers to a saturated hydrocarbon containing 2 to 4 carbon atoms having at least one carbon-carbon triple bond. The term includes both straight and branched moieties. Representative examples of C₃₋₆ alkynyl include, but are not limited to, ethynyl, 1-propynyl, 2-propynyl. 2-butynyl and 3-butynyl.

The term “C₁₋₄ alkoxy” or “C₁₋₆ alkoxy” as used herein refers to —OR^{π}, wherein R^{π} represents a C₁₋₄ alkyl group or C₁₋₆ alkyl group, respectively, as defined herein. Representative examples of C₁₋₄ alkoxy include, but are not limited to, methoxy, ethoxy, propoxy, iso-propoxy, and butoxy. Representative examples of C₁₋₆ alkoxy include, but are not limited to, ethoxy, propoxy, iso-propoxy, and butoxy.

The term “C₃₋₈ cycloalkyl” as used herein refers to a saturated carbocyclic molecule wherein the cyclic framework has 3 to 8 carbons. Representative examples of C₃₋₈ cycloalkyl include, but are not limited to, cyclopropyl and cyclobutyl.

The term “deutero” as used herein as a prefix to another term for a chemical group refers to a modification of the chemical group, wherein one or more hydrogen atoms are substituted with deuterium (“D” or “²H”). For example, the term “C₁₋₄ deuteroalkyl” refers to a C₁₋₄ alkyl as defined herein, wherein one or more hydrogen atoms are substituted with D. Representative examples of C₁₋₄ deuteroalkyl include, but are not limited to, —CH₂D, —CHD₂, —CD₃, —CH₂CD₃, —CDHCD₃, —CD₂CD₃, —CH(CD₃)₂, —CD(CHD₂)₂, and —CH(CH₂D)(CD₃).

The term “halogen” as used herein refers to —F, —CI, —Br, or —I.

The term “halo” as used herein as a prefix to another term for a chemical group refers to a modification of the chemical group, wherein one or more hydrogen atoms are substituted with a halogen as defined herein. The halogen is independently selected at each occurrence. For example, the term “C₁₋₄ haloalkyl” refers to a C₁₋₄ alkyl as defined herein, wherein one or more hydrogen atoms are substituted with a halogen. Representative examples of C₁₋₄ haloalkyl include, but are not limited to, —CH₂F, —CHF₂, —CF₃, —CHFCl, —CH₂CF₃, —CFHCF₃, —CF₂CF₃, —CH(CF₃)₂, —CF(CHF₂)₂, and —CH(CH₂F)(CF₃).

As used herein, the term “heteroaryl” refers to a 5-20 membered monocyclic- or bicyclic- or tricyclic-aromatic ring system, having 1 to 8 heteroatoms selected from N, O and S. In certain preferred aspects, the heteroaryl is a 5-10 membered ring system (e.g., 5-7 membered monocycle, an 8-10 membered bicycle or a 11-14 membered tricycle) or a 5-7 membered ring system. Exemplary monocyclic heteroaryl groups include 2- or 3-thienyl, 2- or 3-furyl, 2- or 3-pyrrolyl, 2-, 4-, or 5-imidazolyl, 3- , 4-, or 5-pyrazolyl, 2- , 4-, or 5-thiazolyl, 3- , 4-, or 5-isothiazolyl, 2- , 4-, or 5-oxazolyl, 3- , 4-, or 5-isoxazolyl, 3- or 5-1,2,4-triazolyl, 4- or 5-1,2,3-triazolyl, tetrazolyl, 2- , 3-, or 4-pyridyl, 3- or 4-pyridazinyl, 3- , 4-, or 5-pyrazinyl, 2-pyrazinyl, and 2- , 4-, and 5-pyrimidinyl. Exemplary bicyclic heteroaryl groups include 1-, 3- , 4-, 5-, 6-, 7-, or 8-isoquinolinyl, 2- , 3-, 4-, 5-, 6-, 7-, or 8-quinolinyl, 1-, 3- , 4-, 5-, 6-, 7-, or 8-isoquinolinyl, 1-, 2- , 4-, 5-, 6-, 7-, or 8-benzimidazolyl and 1-, 2- , 3-, 4-, 5-, 6-, 7-, or 8-indolyl.

The term “heteroaryl” also refers to a group in which a heteroaromatic ring is fused to one or more aryl, cycloaliphatic, or heterocyclyl rings.

As used herein, the term “heterocycle,” “heterocycloalkyl” or “heterocyclo” refers to a saturated or unsaturated non-aromatic ring or ring system, e.g., which is a 4-, 5-, 6-, or 7-membered monocyclic, 7-, 8-, 9-, 10-, 11-, or 12-membered bicyclic or 10-, 11-, 12-, 13-, 14- or 15-membered tricyclic ring system and contains at least one heteroatom selected from O, S and N, where the N and S can also optionally be oxidized to various oxidation states. The heterocyclic group can be attached at a heteroatom or a carbon atom. The heterocyclyl can include fused or bridged rings as well as spirocyclic rings. Examples of heterocycles include tetrahydrofuran, dihydrofuran, 1,4-dioxane, morpholine, 1,4-dithiane, piperazine, piperidine, 1,3-dioxolane, imidazolidine, imidazoline, pyrroline, pyrrolidine, tetrahydropyran, dihydropyran, oxathiolane, dithiolane, 1,3-dioxane, 1,3-dithiane, oxathiane, thiomorpholine, azetidine, thiazolidine, morpholine, and the like.

The term “pharmaceutically acceptable” as used herein refers to generally recognized for use in subjects, particularly in humans.

The term “pharmaceutically acceptable salt” as used herein refers to a salt of a compound that is pharmaceutically acceptable and that possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, and the like; or (2) salts formed when an acidic proton present in the parent compound either is replaced by a metal ion, for example, an alkali metal ion, an alkaline earth ion, or an aluminum ion; or coordinates with an organic base such as ethanolamine, diethanolamine, triethanolamine, N-methylglucamine, dicyclohexylamine, and the like. Additional examples of such salts can be found in Berge et al., J. Pharm. Sci. 66(1):1-19 (1977). See also Stahl et al., Pharmaceutical Salts: Properties, Selection, and Use, 2^{nd} Revised Edition (2011).

The term “pharmaceutically acceptable excipient” as used herein refers to a broad range of ingredients that may be combined with a compound or salt disclosed herein to prepare a pharmaceutical composition or formulation. Typically, excipients include, but are not limited to, diluents, colorants, vehicles, anti-adherents, glidants, disintegrants, flavoring agents, coatings, binders, sweeteners, lubricants, sorbents, preservatives, and the like.

The term “subject” as used herein refers to humans and mammals, including, but not limited to, primates, cows, sheep, goats, horses, dogs, cats, rabbits, rats, and mice. In one embodiment the subject is a human.

The term “therapeutically effective amount” as used herein refers to that amount of a compound disclosed herein that will elicit the biological or medical response of a tissue, a system, or subject that is being sought by a researcher, veterinarian, medical doctor or other clinician.

### General Synthetic Procedures

The compounds provided herein can be synthesized according to the procedures described in this and the following sections. The synthetic methods described herein are merely exemplary, and the compounds disclosed herein may also be synthesized by alternate routes utilizing alternative synthetic strategies, as appreciated by persons of ordinary skill in the art. It should be appreciated that the general synthetic procedures and specific examples provided herein are illustrative only and should not be construed as limiting the scope of the present disclosure in any manner.

Generally, the compounds of Formula I can be synthesized according to the following schemes. Any variables used in the following schemes are the variables as defined for Formula I, unless otherwise noted. All starting materials are either commercially available, for example, from Merck Sigma-Aldrich Inc., Fluorochem Ltd, and Enamine Ltd. or known in the art and may be synthesized by employing known procedures using ordinary skill. Starting material may also be synthesized via the procedures disclosed herein. Suitable reaction conditions, such as, solvent, reaction temperature, and reagents, for the Schemes discussed in this section, may be found in the examples provided herein.

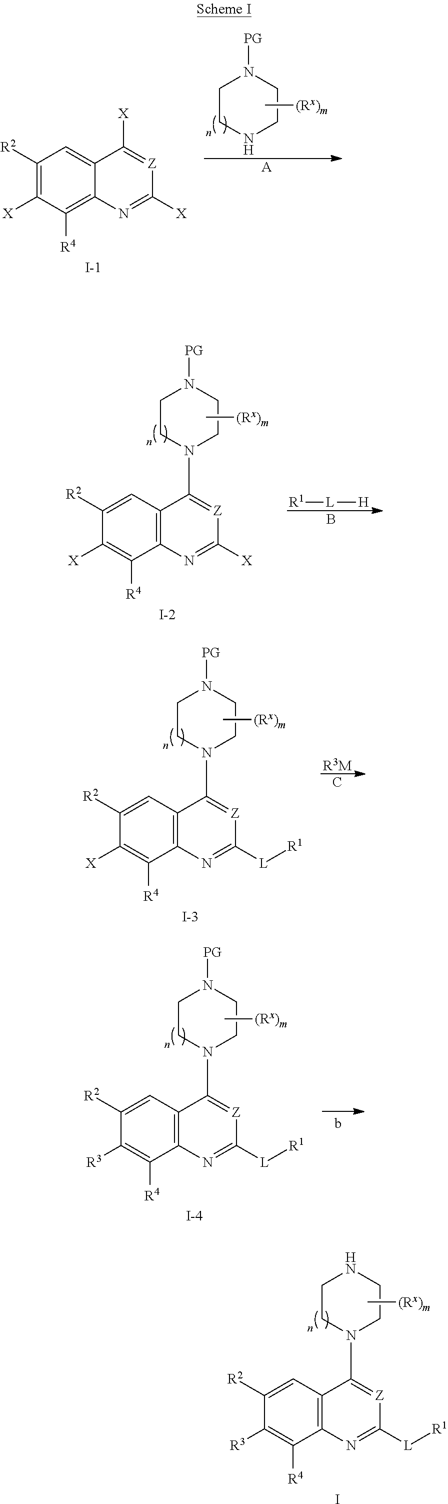

Compounds of Formula (I) can be prepared according to Scheme I. In step A, compound (I-1) undergoes S_{N}Ar reaction with optionally substituted mono-Boc protected amine in a solvent such as acetonitrile and in the presence of a base such as N,N-diisopropylethylamine to give compound (I-2). In step B, compound (I-2) is either pre-treated with a fluoride source such as potassium fluoride, or directly undergoes S_{N}Ar reaction with a nucleophile having the formula R¹—L—H in a solvent such as dimethylsulfoxide, or mixture of solvents such as tetrahydrofuran and N,N-dimethylforamide, in the presence of a base such as sodium hydride or cesium carbonate, with or without a nucleophilic catalyst such as 1,4-diazabicyclo[2.2.2]octane, to give compound (I-3). In step C, compound (I-3) is coupled with an organometallic reagent such as a boronic acid (ester) to provide compound (I-4). This coupling reaction proceeds in a solvent such as 1,4-dioxane and a catalyst such as Pd(dppf)Cl₂, with or without a base such as potassium phosphate. In step D, protecting groups are removed using conditions known in the art. For example, Boc can be removed with TFA or HCl. Silyl groups can be removed using a fluoride source.

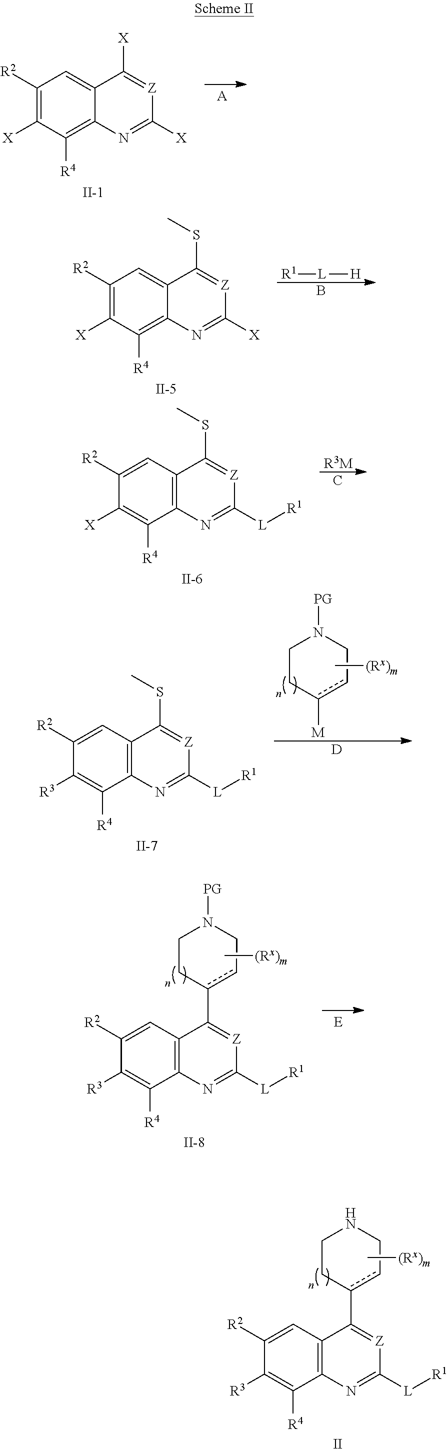

Compounds of Formula (II) can be prepared according to Scheme II. In step A, compound (II-5) is treated with sodium thiomethoxide in a solvent such as tetrahydrofuran to give compound (II-6). In step B, compound (II-6) is either pre-treated with a fluoride source such as potassium fluoride, or directly undergoes S_{N}Ar reaction with a nucleophile having the formula R¹—L—H in a solvent such as dimethylsulfoxide, or mixture of solvents such as tetrahydrofuran and N,N-dimethylforamide, in the presence of a base such as sodium hydride or cesium carbonate, with or without a nucleophilic catalyst such as 1,4-diazabicyclo[2.2.2]octane, to give compound (II-7). In step C, compound (II-7) is coupled with an organometallic reagent such as a boronic acid (ester) to provide compound (II-8). This coupling reaction proceeds in a solvent such as 1,4-dioxane and a catalyst such as Pd(dppf)Cl₂, with or without a base such as potassium phosphate. In step D, compound (II-8) is coupled with an organometallic reagent, such as a boronic acid (ester) to give compound (II-9). This coupling reaction proceeds in a solvent such as 1,4-dioxane and a catalyst such as Pd(PPh₃)₄, in the presence of an additive such as CuTC, with or without a base such as potassium phosphate. In step E, protecting groups are removed using conditions known in the art. For example, Boc can be removed with TFA or HCl. Silyl groups can be removed using a fluoride source.

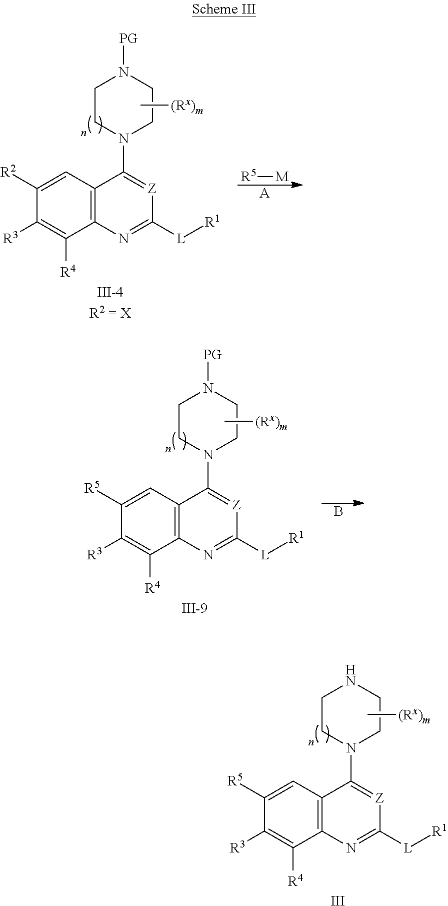

Compounds of Formula (III) can be prepared according to Scheme III. In step A, compound (4) is coupled with a nucleophile or an organometallic reagent such as a boronic acid (ester) to provide compound (9). This coupling reaction proceeds in a solvent such as 1,4-dioxane and a catalyst such as SPhos Pd G3, with or without a base such as potassium phosphate. In step B, protecting groups are removed using conditions known in the art. For example, Boc can be removed with TFA or HCl. Silyl groups can be removed using a fluoride source.

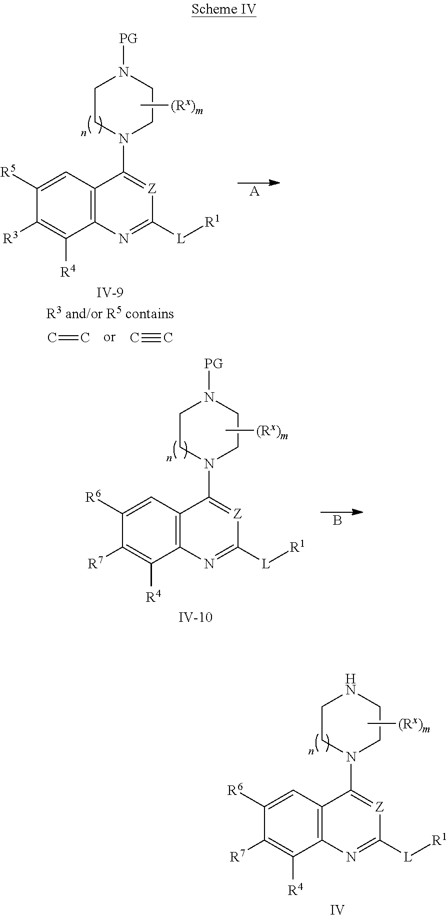

Compounds of Formula (IV) can be prepared according to Scheme IV. In step A, compound (IV-9) is reduced using a reductant such as hydrogen gas, with a catalyst such as Pd/C, in a solvent such as ethanol, to give compound (IV-10). In step B, protecting groups are removed using conditions known in the art. For example, Boc can be removed with TFA or HCl. Silyl groups can be removed using a fluoride source.

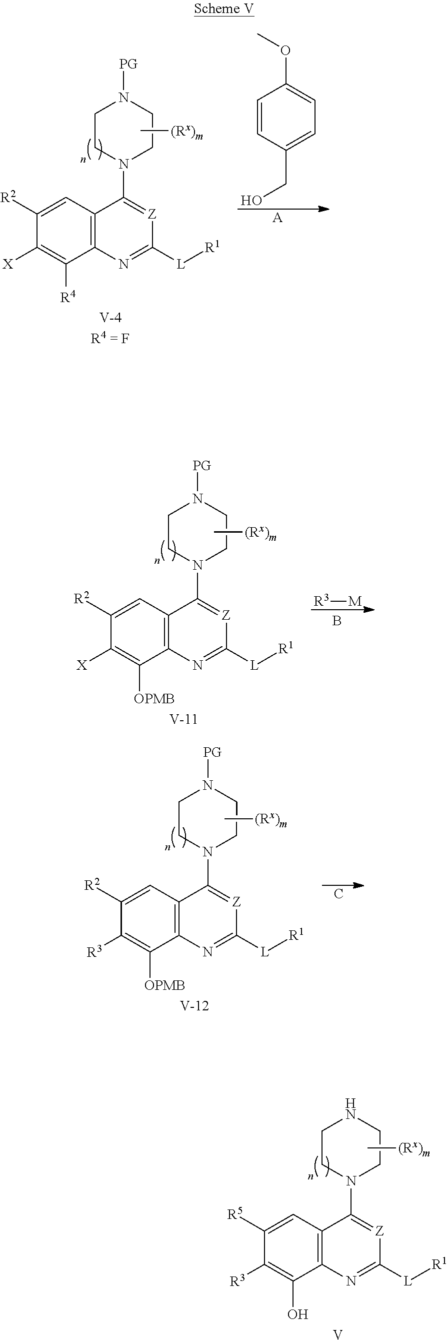

Compounds of Formula (V) can be prepared according to Scheme V. In step A, compound (V-4) undergoes S_{N}Ar with 4-methoxybenzyl alcohol, in the presence of a base such as sodium hydride, in a solvent such as tetrahydrofuran to give compound (V-11). In step B, compound (V-11) is coupled with an organometallic reagent such as a boronic acid (ester) to provide compound (V-12). This coupling reaction proceeds in a solvent such as 1,4-dioxane and a catalyst such as Pd(dppf)Cl₂, with or without a base such as potassium phosphate. In step C, protecting groups are removed using conditions known in the art. For example, PMB and Boc can be removed with TFA or HCl. Silyl groups can be removed using a fluoride source.

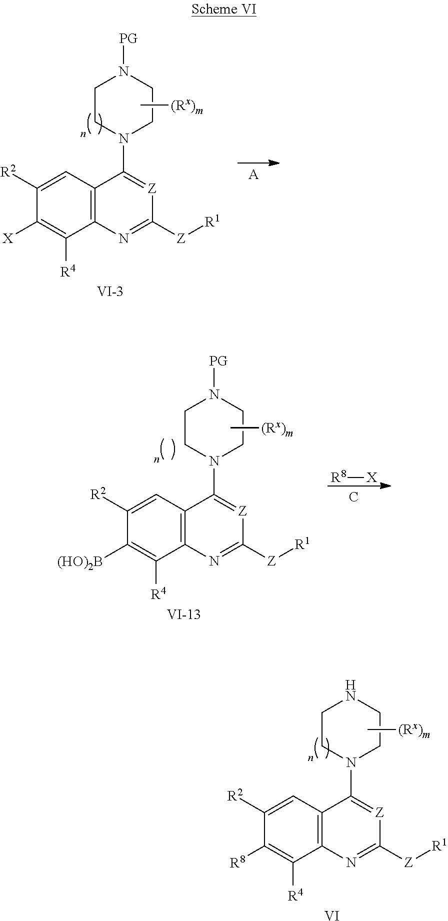

Compounds of Formula (VI) can be prepared according to Scheme VI. In Step A, compound (VI-3) undergoes borylation in the presence of reagents like B₂pin₂, a catalyst such as Pd(dppf)Cl₂, a base such as potassium acetate, in solvents such as 1,4-dioxane. In step B, compound (VI-13) undergoes Pd-catalyzed coupling reactions with an aryl halide to provide. This coupling reaction proceeds in a solvent such as 1,4-dioxane and a catalyst such as XPhos Pd G3, with a base such as potassium phosphate. The resulting compound may undergo further transformation, such as deprotection to give compound (VI).

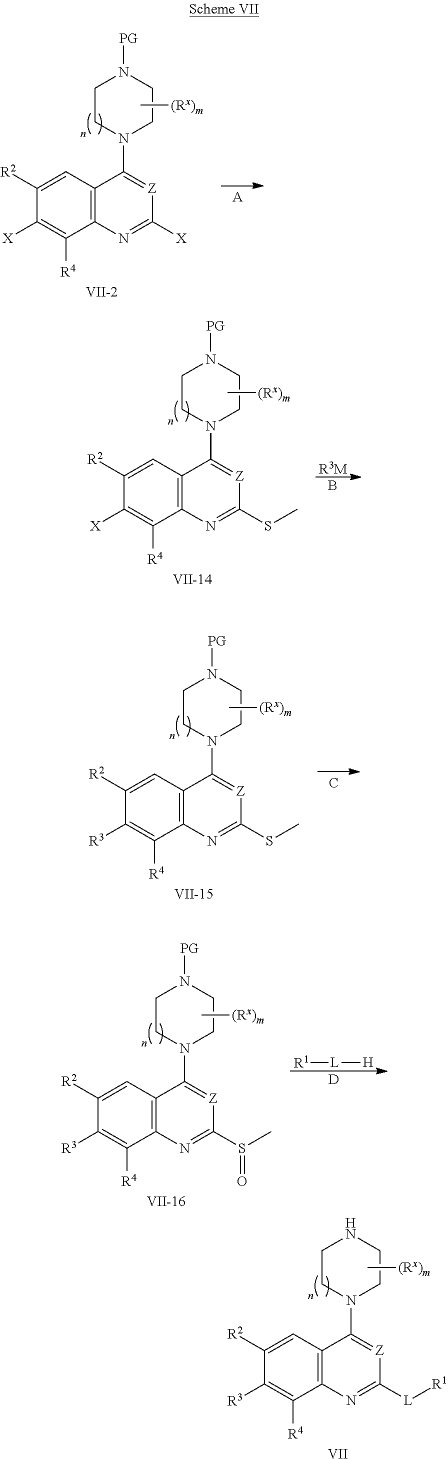

Compounds of Formula (VII) can be prepared according to Scheme VII. In step A, compound (VII-2) is either pre-treated with a fluoride source such as potassium fluoride, or directly undergoes S_{N}Ar reaction with a nucleophile having the formula R¹—L—H in a solvent such as dimethylsulfoxide, or mixture of solvents such as tetrahydrofuran and N,N-dimethylforamide, in the presence of a base such as sodium hydride or cesium carbonate, with or without a nucleophilic catalyst such as 1,4-diazabicyclo[2.2.2]octane, to give compound (VII-14). In step B, compound (VII-14) is coupled with an organometallic reagent such as a boronic acid (ester) to provide compound (VII-15). This coupling reaction proceeds in a solvent such as 1,4-dioxane and a catalyst such as cataCXium A Pd G3, with or without a base such as potassium phosphate. In step C, compound (VII-15) is treated with an oxidizing agent such as 3-chloroperoxybenzoic acid to give compound (VII-16). In step D, compound (VII-16) undergoes S_{N}Ar reaction with a nucleophile having the formula R¹—L—H in a solvent such as tetrahydrofuran in the presence of a base such as potassium tert-butoxide. The resulting product may undergo further transformation, such as deprotection, to provide compound (VII).

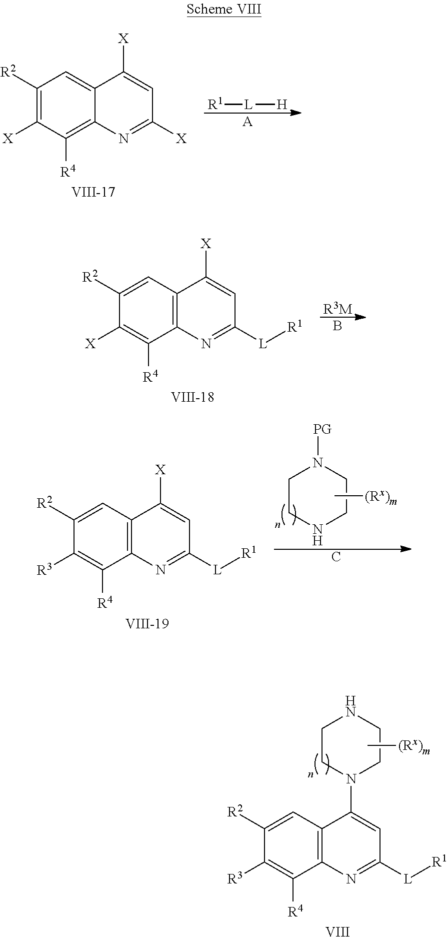

Compounds of Formula (VIII) can be prepared according to Scheme VIII. In step A, compound (VIII-17) undergoes S_{N}Ar reaction with a nucleophile having the formula R¹—L—H in a solvent such as tetrahydrofuran, in the presence of a base such as sodium hydride to give compound (VIII-18). In step B, compound (VIII-18) is coupled with an organometallic reagent such as a boronic acid (ester) to provide compound (VIII-19). This coupling reaction proceeds in a solvent such as 1,4-dioxane and a catalyst such as cataCXium A Pd G3, with or without a base such as potassium phosphate. In Step C, compound (VIII-19) undergoes palladium-catalyzed C—N coupling reaction with optionally substituted mono-Boc protected amine. This coupling reaction proceeds in a solvent such as 1,4-dioxane and a catalyst such as RuPhos Pd G4, with a base such as cesium carbonate. The resulting product may undergo further transformation to provide compound (VIII).

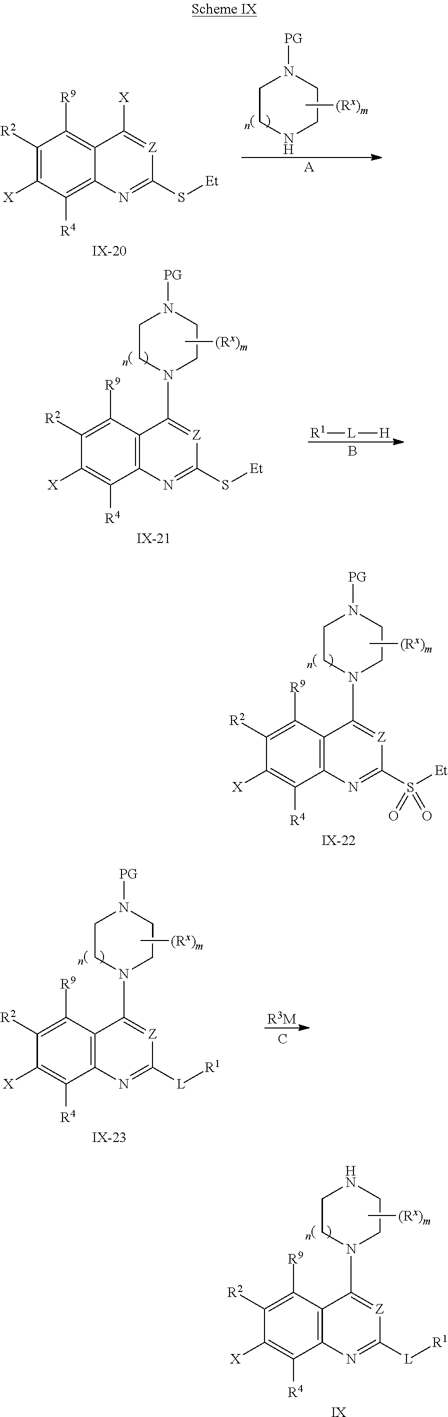

Compounds of Formula (IX) can be prepared according to Scheme IX. In step A, compound (IX-20) undergoes S_{N}Ar reaction with optionally substituted mono-Boc protected amine in a solvent or solvent mixture such as dichloromethane and isopropanol, with or without a base to give compound (IX-21). In step B, compound (IX-21) is treated with an oxidizing agent such as 3-chloroperoxybenzoic acid to give compound (IX-22). In step C, compound (IX-22) is coupled with an organometallic reagent such as a boronic acid (ester). This coupling reaction proceeds in a solvent such as 1,4-dioxane and a catalyst such as cataCXium A Pd G3, with or without a base such as potassium phosphate. The resulting product may undergo further transformation, such as deprotection, to provide compound (IX).

### EXAMPLES

This section provides specific examples of compounds of Formula I and methods of making the same.

### LIST OF ABBREVIATIONS

TABLE 1

| | |
|---|---|
| AcOH | acetic acid |
| aq or aq. | aqueous |
| B₂pin₂ | Bis(pinacolato)diboron |
| BOC or Boc | tert-butyloxycarbonyl |
| BrettPhos Pd G4 | (SP-4-3)-[Dicyclohexyl[3,6-dimethoxy-2′,4′,6′-tris(1- |
| | methylethyl)[1,1′-biphenyl]-2-yl]phosphine- |
| | κP](methanesulfonato-κO)[2′-(methylamino- |
| | κN)[1,1′-biphenyl]-2-yl-κC]palladium |
| cataCXium A Pd | Mesylate[(di(1-adamantyl)-n-butylphosphine)-2-(2′- |
| G3 | amino-1,1′-biphenyl)]palladium(II) |
| COD or cod | 1,5-Cyclooctadiene |
| CuTC | Copper(I)-thiophene-2-carboxylate |
| DABCO | 1,4-diazabicyclo[2.2.2]octane |
| DCM | dichloromethane |
| DMF | N,N-dimethylformamide |
| DMSO | dimethyl sulfoxide |
| Dppf, DPPF or | 1,1′-bis(diphenylphosphino)ferrocene |
| dppf | |
| dtbbpy | 4,4′-Di-tert-butyl-2,2′-dipyridyl |
| eq or eq. or | equivalent |
| equiv. | |
| ESI or ES | electrospray ionization |
| Et | ethyl |
| EtOAc | ethyl acetate |
| g | gram(s) |
| h | hour(s) |
| HBpin | 4,4,5,5-Tetramethyl-1,3,2-dioxaborolane |
| HMPA | Hexamethylphosphoramide |
| HOAc | Acetic acid |
| HPLC | high pressure liquid chromatography |
| iPr | iso-propyl |
| iPr₂NEt or DIPEA | N-ethyl diisopropylamine (Hünig's base) |
| KOAc | potassium acetate |
| LC MS, LCMS, | liquid chromatography mass spectroscopy |
| LC-MS or LC/MS | |
| LHMDS or | lithium hexamethyldisilazide |
| LiHMDS | |
| m/z | mass divided by charge |
| mCPBA | meta-Chloroperoxybenzoic acid |
| Me | methyl |
| MeCN | acetonitrile |
| MeOH | methanol |
| mg | milligrams |
| min | minutes |
| mL | milliliters |
| MS | mass spectra |
| NCS | N-Chlorosuccinimide |
| NMR | nuclear magnetic resonance |
| NIS | N-Iodosuccinimide |
| Pd(dppf)Cl₂•DCM, | [1,1′- |
| Pd(dppf)Cl₂ | bis(diphenylphosphino)ferrocene]dichloropalladium |
| | (II), complex with dichloromethane |
| Pd(dtbpf)Cl₂ | [1,1′-Bis(di-tert- |
| | butylphosphino)ferrocene]dichloropalladium(II) |
| Pd(PPh₃)₄ | tetrakis(triphenylphosphine)palladium(0) |
| Ph | Phenyl |
| PhMe | Toluene |
| pin | pinacolato |
| PMB | 4-Methoxybenzyl |
| Py | pyridine |
| rbf | round-bottom flask |
| RP-HPLC | reverse phase high pressure liquid chromatography |
| RT or rt or r.t. | room temperature |
| RuPhos Pd G4 | (SP-4-3)-[[2′,6′-Bis(1-methylethoxy)[1,1′-biphenyl]- |
| | 2-yl]dicyclohexylphosphine-κP](methanesulfonato- |
| | κO)[2′-(methylamino-κN)[1,1′-biphenyl]-2-yl- |
| | κC]palladium |
| sat. or satd. | saturated |
| SFC | supercritical fluid chromatography |
| TBAF | tetra-n-butylammonium fluoride |
| TBDPS | tert-Butyldiphenylsilyl |
| TBDPSCl | tert-Butyldiphenylsilyl chloride |
| tBuXPhos Pd G3 | [(2-Di-tert-butylphosphino-2′,4′,6′-triisopropyl-1,1′- |
| | biphenyl)-2-(2′-amino-1,1′-biphenyl)] palladium(II) |
| | methanesulfonate |
| TC | Thiophene-2-carboxylate |
| TEA or Et₃N | triethylamine |
| Tf | trifluoromethanesulfonate |
| TFA | trifluoroacetic acid |
| THF | tetrahydrofuran |
| TMEDA | N,N,N′,N′-Tetramethylethylenediamine |
| TIPS | triisopropylsilyl |
| UV | Ultraviolet |
| XPhos | 2-dicyclohexylphosphino-2′,4′,6′-triisopropyl-1,1′- |
| | biphenyl |
| Xphos Pd G2 | chloro(2-dicyclohexylphosphino-2′,4′,6′-triisopropyl- |
| | 1,1′-biphenyl)[2-(2′-amino-1,1′-biphenyl)]palladium |
| | (II) |
| XPhos Pd G3 | (2-Dicyclohexylphosphino-2′,4′,6′-triisopropyl-1,1′- |
| | biphenyl)[2-(2′-amino-1,1′-biphenyl)]palladium(II) |
| | methanesulfonate |

### General Analytical and Purification Methods

Provided in this section are descriptions of the general analytical and purification methods used to prepare the specific examples provided herein.

Chromatography: Unless otherwise indicated, crude product-containing residues were purified by passing the crude material or concentrate through either a Biotage or ISCO brand silica gel column pre-packed with flash silica (SiO2) and eluting the product off the column with a solvent gradient as indicated.

Preparative HPLC Method: Where so indicated, the compounds described herein were purified via reverse phase HPLC using Waters FractionLynx semi-preparative HPLC-MS system utilizing one of the following two HPLC columns: (a) Phenomenex Gemini column (5 micron, C18, 150×30 mm) or (b) Waters X-select CSH column (5 micron, C18, 100×30 mm). A typical run through the instrument included: eluting at 45 mL/min with a linear gradient of 10%(v/v) to 100% MeCN (0.1% v/v formic acid) in water (0.1% formic acid) over 10 minutes; conditions can be varied to achieve optimal separations.

Proton NMR Spectra: Unless otherwise indicated, all 1H NMR spectra were collected on a Bruker NMR Instrument at 300, 400 or 500 MHz. Where so characterized, all observed protons are reported as parts-per-million (ppm) downfield from tetramethylsilane (TMS) using the internal solvent peak as reference.

Mass Spectra (MS): Unless otherwise indicated, all mass spectral data for starting materials, intermediates and/or exemplary compounds are reported as mass/charge (m/z), having an [M+H]⁺ molecular ion. The molecular ion reported was obtained by electrospray detection method (commonly referred to as an ESI MS) utilizing a Waters Acquity UPLC/MS system. Compounds having an isotopic atom, such as bromine and the like, are generally reported according to the detected isotopic pattern, as appreciated by those skilled in the art.

### Preparation of Intermediates

### ((2R,7aR)-2-Fluorotetrahydro-1H-pyrrolizin-7a(5H)-yl)methanol (Intermediate A

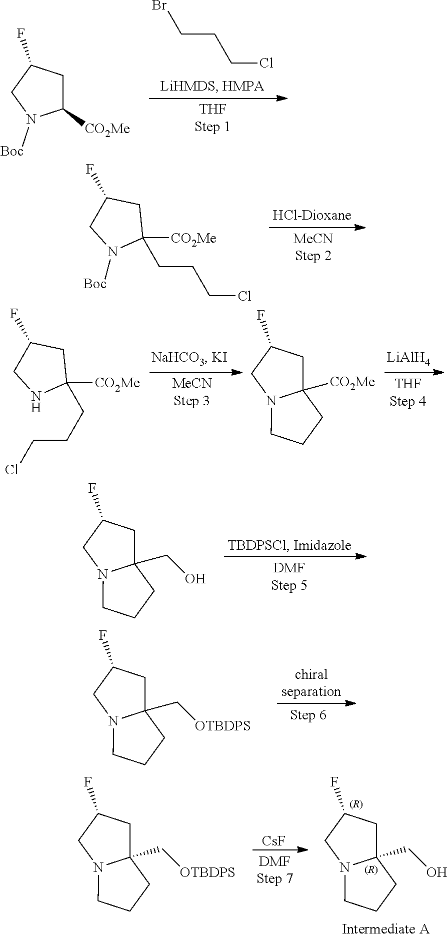

Step 1: 1-(tert-Butyl) 2-methyl (4R)-2-(3-chloropropyl)-4-fluoropyrrolidine-1,2-dicarboxylate. To a mixture of 1-tert-butyl 2-methyl (2S,4R)-4-fluoropyrrolidine-1,2-dicarboxylate (45.0 g, 182 mmol) and HMPA (42.4 g, 237 mmol, 41.6 mL) in THF (250 mL) was added LiHMDS (1.0 M, 237 mL) in portions at −70° C. under N₂. The mixture was stirred at −70° C. for 1 h. Then to the mixture was added 1-bromo-3-chloro-propane (143.3 g, 910 mmol, 90 mL) in portions at −70° C. under N₂. The resulting mixture was warmed to 15° C. and stirred for 5 h. TLC indicated the starting material was consumed completely. The mixture was poured into aq. NH₄Cl (1-L) and stirred for 20 min. The aqueous phase was extracted with EtOAc (500 mL×3). The combined organic phase was dried over anhydrous Na₂SO₄, filtered and concentrated under vacuum. The residue was purified by column chromatography on silica gel, eluting with petroleum ether/EtOAc (10/1 to 1/1) to give 1-(tert-butyl) 2-methyl (4R)-2-(3-chloropropyl)-4-fluoropyrrolidine-1,2-dicarboxylate (68.0 g, 210 mmol, 57% yield) as yellow oil.

Step 2: Methyl (4R)-2-(3-chloropropyl)-4-fluoropyrrolidine-2-carboxylate. To a mixture of 1-(tert-butyl) 2-methyl (4R)-2-(3-chloropropyl)-4-fluoropyrrolidine-1,2-dicarboxylate (34.0 g, 105 mmol) in CH₃CN (200 mL) was added HCl/dioxane (4 M, 150 mL) in one portion at 15° C. under N₂. The mixture was stirred at 15° C. for 2 h. TLC indicated the material was consumed completely. The mixture was concentrated under reduced pressure at 45° C. Crude methyl (4R)-2-(3-chloropropyl)-4-fluoro-pyrrolidine-2-carboxylate (55.0 g, crude) was obtained and used directly in the next step.

Step 3: Methyl (2R)-2-fluorotetrahydro-1H-pyrrolizine-7a(5H)-carboxylate. To a mixture of methyl (4R)-2-(3-chloropropyl)-4-fluoro-pyrrolidine-2-carboxylate (55.0 g, 211 mmol) in CH₃CN (550 mL) was added NaHCO₃ (88.8 g, 1.06 mol, 41 mL) and KI (3.51 g, 21.1 mmol) in one portion at 15° C. under N₂. The mixture was stirred at 50° C. for 12 h. TLC indicated the material was consumed completely and one new spot formed. The mixture was filtered and the filter cake was washed with EtOAc (100 mL×3). The filtrate was concentrated in vacuum. The residue was purified by column chromatography on silica gel, eluting with petroleum ether/EtOAc (3/1 to 0/1) to give methyl (2R)-2-fluorotetrahydro-1H-pyrrolizine-7a(5H)-carboxylate (27.0 g, 144 mmol, 49% yield) as yellow oil.

Step 4: ((2R)-2-Fluorotetrahydro-1H-pyrrolizin-7a(5H)-yl)methanol. To a mixture of methyl (2R)-2-fluorotetrahydro-1H-pyrrolizine-7a(5H)-carboxylate (10.0 g, 53.4 mmol) in THF (100 mL) was added LiAlH₄ (4.05 g, 107 mmol) in portions at −40° C. Then the mixture was stirred at −40° C. for 1 h. TLC showed the reaction was completed. To the reaction mixture was added Na₂SO₄·10 H₂O (20 g) slowly in portions at 0° C. The mixture was diluted with THF (50 mL) and filtered. The filter cake was washed with THF (300 mL). The organic layer was concentrated under vacuum. Crude ((2R)-2-fluorotetrahydro-1H-pyrrolizin-7a(5H)-yl)methanol (10.0 g, crude) as colorless oil was obtained and used directly in the next step.

Step 5: (2R)-7a-(((tert-Butyldiphenylsilyl)oxy)methyl)-2-fluorohexahydro-1H-pyrrolizine. To a mixture of ((2R)-2-fluorotetrahydro-1H-pyrrolizin-7a(5H)-yl)methanol (20.0 g, 126 mmol) and imidazole (34.2 g, 503 mmol) in DMF (25 mL) was added TBDPSCl (69.1 g, 251 mmol, 64.54 mL). Then the mixture was stirred at 20° C. for 3 h. TLC (EtOAc:MeOH=6:1) showed the reaction was complete. The mixture was diluted with H₂O (100 mL) and extracted with EtOAc (400 mL×2). The combined organic layers were washed with brine (150 mL), dried over Na₂SO₄, filtered and concentrated under reduced pressure. The resulting residue was purified by column chromatography on silica gel, eluting with petroleum ether/EtOAc (20/1 to 10/1) to give (2R)-7a-(((tert-butyldiphenylsilyl)oxy)methyl)-2-fluorohexahydro-1H-pyrrolizine (11.0 g, 17.7 mmol, 26% yield over two steps) as colorless oil. m/z (ESI, +ve ion): 398.3 (M+H)⁺.

Step 6: (2R,7aR)-7a-(((tert-Butyldiphenylsilyl)oxy)methyl)-2-fluorohexahydro-1H-pyrrolizine. (2R)-7a-(((tert-butyldiphenylsilyl)oxy)methyl)-2-fluorohexahydro-1H-pyrrolizine (6.50 g, 16.4 mmol) was separated by column chromatography on silica gel, eluting with petroleum ether/EtOAc (7/1 to 0/1) to give (2R,7aR)-7a-(((tert-butyldiphenylsilyl)oxy)methyl)-2-fluorohexahydro-1H-pyrrolizine (2.35 g, 5.66 mmol, 36% yield, 96% purity) as yellow oil.

Step 7: ((2R,7aR)-2-Fluorotetrahydro-1H-pyrrolizin-7a(5H)-yl)methanol. To a mixture of (2R,7aR)-7a-(((tert-butyldiphenylsilyl)oxy)methyl)-2-fluorohexahydro-1H-pyrrolizine (500 mg, 1.26 mmol) in DMF (5 mL) was added CsF (1.91 g, 12.6 mmol) in one portion at 15° C. under N₂. The mixture was stirred at 70° C. for 72 h. Another batch with 9 g of (2R,7aR)-7a-(((tert-butyldiphenylsilyl)oxy)methyl)-2-fluorohexahydro-1H-pyrrolizine was set up following the same procedure. The two batches were combined for the purification. The crude product was purified by column chromatography on silica gel, eluting with EtOAc/methanol (6/1 to 1/1, with NH₃·H₂O additive) to give ((2R,7aR)-2-fluorotetrahydro-1H-pyrrolizin-7a(5H)-yl)methanol (2.60 g, 16.3 mmol, 68% yield) as yellow oil. ¹H NMR (400 MHz, CHLOROFORM-d) δ ppm 5.22 (dt, J=54 Hz, 4.6 Hz, 1H), 3.47-3.31 (m, 3H), 3.00-2.98 (m, 1H), 2.88-2.75 (m, 1H), 2.67-2.65 (m, 1H), 2.25-2.15 (m, 1H), 1.92-1.80 (m, 4H), 1.60-1.55 (m, 1H).

### Triisopropyl((8-(4,4,5,5-tetramethyl-1,3,2-dioxaborolan-2-yl)naphthalen-1-yl)ethynyl)silane (Intermediate B

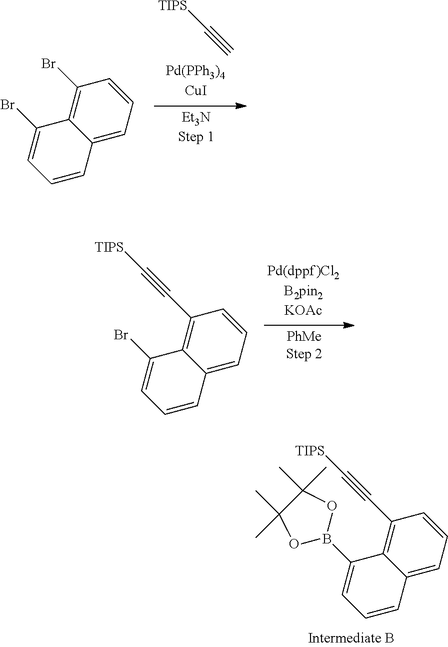

Step 1: ((8-Bromonaphthalen-1-yl)ethynyl)triisopropylsilane. A 40-mL vial was charged with triethylamine (7.43 g, 10.3 mL, 73.4 mmol), tetrakis(triphenylphosphine)palladium(0) (0.30 g, 0.26 mmol), copper(I) iodide (0.10 g, 0.53 mmol), 1,8-dibromonaphthalene (0.75 g, 2.62 mmol), and (triisopropylsilyl)acetylene (0.53 g, 0.65 mL, 2.88 mmol). The reaction was purged with nitrogen for 5 min and then stirred at 80° C. After 6 h, water (20 mL) and EtOAc (20 mL) were added. The mixture was transferred to a separatory funnel. The organic layer was separated, washed with saturated ammonium chloride solution, dried over anhydrous Na₂SO₄, and concentrated under reduced pressure. The resulting crude product was purified by column chromatography on silica gel eluting with a gradient of 0-20% EtOAc in heptane to yield ((8-bromonaphthalen-1-yl)ethynyl)triisopropylsilane (0.99 g, 2.56 mmol, 98% yield). m/z (ESI, +ve ion): 387.1 (M+H)⁺. ¹H NMR (400 MHz, DMSO-d6) δ ppm 7.99-8.14 (m, 2H), 7.78-7.99 (m, 2H), 7.51-7.61 (m, 1H), 7.38-7.48 (m, 1H), 1.10-1.17 (m, 18H), 0.81-0.89 (m, 3H).

Step 2: Triisopropyl((8-(4,4,5,5-tetramethyl-1,3,2-dioxaborolan-2-yl)naphthalen-1-yl)ethynyl)silane. A 20-mL vial was charged with [1,1′-bis(diphenylphosphino)ferrocene]dichloropalladium(II) (0.11 g, 0.16 mmol), bis(pinacalato)diboron (0.26 mg, 1.03 mmol), potassium acetate (0.15 g, 1.55 mmol) and ((8-bromonaphthalen-1-yl)ethynyl)triisopropylsilane (0.20 g, 0.52 mmol). The solids were suspended in toluene (4.7 mL). The reaction was purged with nitrogen for 5 min and then stirred at 80° C. After 16 h, the reaction was cooled to room temperature and then concentrated under reduced pressure. The crude material was purified by column chromatography on silica gel, eluting with a gradient of 0-15% EtOAc in heptane to provide triisopropyl((8-(4,4,5,5-tetramethyl-1,3,2-dioxaborolan-2-yl)naphthalen-1-yl)ethynyl)silane (0.15 g, 0.34 mmol, 66% yield). m/z (ESI, +ve ion): 435.3 (M+H)⁺

TABLE 1
Additional Intermediates Prepared in an Analogous Manner to Step 2 in 5 Intermediate B.

| | | | MS |
|---|---|---|---|
| Int. # | Structure | Name | m/z (ESI, +ve ion) |
| Intermediate C | 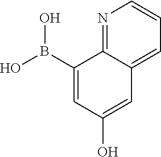 [SMILES: OB(O)c1cc(O)cc2cccnc12] | (6-hydroxyquinolin-8-yl)boronic acid | 190.0 (M + H)⁺ |
| Intermediate D | 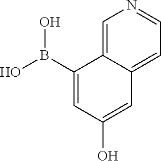 [SMILES: OB(O)c1cc(O)cc2ccncc12] | (6-hydroxyisoquinolin-8- yl)boronic acid | 190.1 (M + H)⁺ |
| Intermediate E | 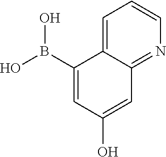 [SMILES: OB(O)c1cc(O)cc2ncccc12] | (7-hydroxyquinolin-5-yl)boronic acid | 190.0 (M + H)⁺ |
| Intermediate F | 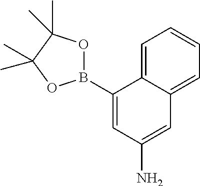 [SMILES: CC1(C)OB(c2cc(N)cc3ccccc23)OC1(C)C] | 4-(4,4,5,5-tetramethyl-1,3,2- dioxaborolan-2-yl)naphthalen-2- amine | 270.1 (M + H)⁺ |
| Intermediate G | 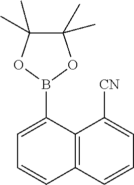 [SMILES: CC1(C)OB(c2cccc3cccc(C#N)c23)OC1(C)C] | 8-(4,4,5,5-tetramethyl-1,3,2- dioxaborolan-2-yl)-1- naphthonitrile | 280.0 (M + H)⁺ |
| Intermediate H | 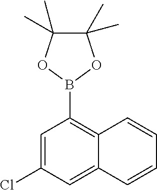 [SMILES: CC1(C)OB(c2cc(Cl)cc3ccccc23)OC1(C)C] | 2-(3-chloronaphthalen-1-yl)- 4,4,5,5-tetramethyl-1,3,2- dioxaborolane | |

### Triisopropyl((6-(methoxymethoxy)-8-(4,4,5,5-tetramethyl-1,3,2-dioxaborolan-2-yl)naphthalen-1-yl)ethynyl)silane (Intermediate I

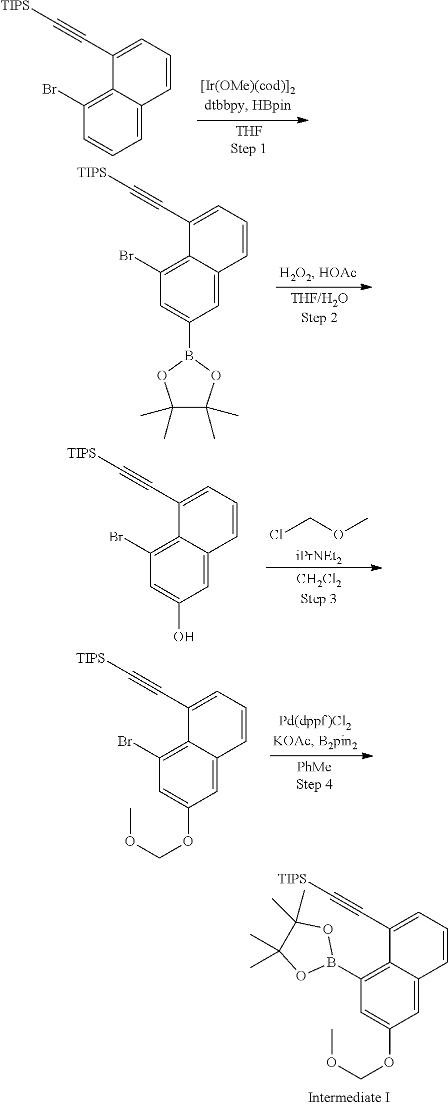

Step 1: ((8-Bromo-6-(4,4,5,5-tetramethyl-1,3,2-dioxaborolan-2-yl)naphthalen-1-yl)ethynyl)triisopropylsilane. A 20-mL vial was charged with ((8-bromonaphthalen-1-yl)ethynyl)triisopropylsilane (0.34 g, 0.88 mmol), 4,4,5,5-tetramethyl-1,3,2-dioxaborolane (0.28 g, 0.32 mL, 2.19 mmol, Sigma-Aldrich Corporation), 4,4′-di-tert-butyl-2,2′-dipyridyl (28.3 mg, 0.11 mmol, Sigma-Aldrich Corporation) and (1,5-cyclooctadiene)(methoxy)iridium(I) dimer (58 mg, 0.088 mmol, Sigma-Aldrich Corporation). The reaction was purged with nitrogen for 5 min and then stirred at 60° C. for 1 h. The solution was concentrated under reduced pressure. The crude material was absorbed onto a plug of silica gel and purified by column chromatography on silica gel, eluting with a gradient of 0-50% EtOAc in hexane to provide the product, which was used in the next step without further manipulation.

Step 2: 4-Bromo-5-((triisopropylsilyl)ethynyl)naphthalen-2-ol. To a 50-mL round-bottomed flask was added ((8-bromo-6-(4,4,5,5-tetramethyl-1,3,2-dioxaborolan-2-yl)naphthalen-1-yl)ethynyl)triisopropylsilane (0.45 g, 0.88 mmol) in tetrahydrofuran (3.3 mL) and water (1.1 mL). Hydrogen peroxide (0.90 g, 0.81 mL, 7.91 mmol, Sigma-Aldrich) was added slowly at 0° C., followed by acetic acid (2.51 mL, 43.9 mmol). The reaction was stirred for 1 h. The reaction mixture was diluted with saturated Na₂S₂O₃ solution (15 mL) and extracted with EtOAc (2×15 mL). The organic layer was separated and concentrated under reduced pressure. The crude material was absorbed onto a plug of silica gel and purified by column chromatography on silica gel, eluting with a gradient of 0-40% EtOAc in hexane to provide a mixture of isomers. Another batch was prepared via the same method. A total of 600 mg of mixture of isomers was purified by SFC using a Chiralpak AD 21×250 mm, 5 micron, mobile phase of 10% 2-propanol using a flow-rate of 80 mL/min to generate 280 mg of desired product with chemical purity of >95%. Peak assignment determined by SFC with Chiralpak AD and 15% 2-propanol.

Step 3: ((8-Bromo-6-(methoxymethoxy)naphthalen-1-yl)ethynyl)triisopropylsilane. To a 250-mL round-bottomed flask was added 4-bromo-5-((triisopropylsilyl)ethynyl)naphthalen-2-ol (0.35 g, 0.87 mmol) and DIEA (0.34 g, 0.45 mL, 2.60 mmol, Sigma-Aldrich) in DCM (4.3 mL). Chlormethyl methyl ether (0.14 g, 0.13 mL, 1.74 mmol, Sigma-Aldrich Corporation) was added slowly at 0° C. The reaction was stirred for 90 min. The crude material was absorbed onto a plug of silica gel and purified by column chromatography on silica gel column, eluting with a gradient of 0-30% EtOAc in heptane to provide the product. ¹H NMR (400 MHz, CHLOROFORM-d) δ ppm 7.77-7.61 (m, 3H), 7.38-7.22 (m, 2H), 5.27-5.32 (m, 2H), 3.52-3.55 (m, 3H), 1.13-1.28 (m, 21H).

Step 4: Triisopropyl((6-(methoxymethoxy)-8-(4,4,5,5-tetramethyl-1,3,2-dioxaborolan-2-yl)naphthalen-1-yl)ethynyl)silane. A 20-mL vial was charged with ((8-bromo-6-(methoxymethoxy)naphthalen-1-yl)ethynyl)triisopropylsilane (0.13 g, 0.30 mmol), bis(pinacalato)diboron (0.15 g, 0.60 mmol, Combi-Blocks Inc.), potassium acetate (88 mg, 0.90 mmol, Sigma-Aldrich Corporation), 1,1′-bis(diphenylphosphino)ferrocene-palladium dichloride (65 mg, 0.090 mmol, Sigma-Aldrich Corporation) and toluene (4.0 mL). The reaction mixture was purged with nitrogen for 5 min and then stirred at 80° C. for 12 h. The crude material was absorbed onto a plug of silica gel and purified by column chromatography on silica gel column, eluting with a gradient of 0-30% EtOAc in hexane to provide the product.

### 5-Chloro-4-(trimethylstannyl)isoquinoline (Intermediate J

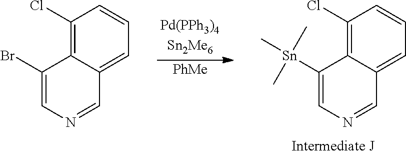

An 8-mL vial was charged with Pd(PPh₃)₄(90 mg, 0.078 mmol) and 4-bromo-5-chloroisoquinoline (0.19 g, 0.78 mmol). The vial was purged with nitrogen for 5 min and then the solids were suspended in degassed toluene (3.9 mL). Hexamethylditin (0.84 g, 0.53 mL, 2.55 mmol) was added. The reaction was then heated to 100° C. and stirred for 30 h. The reaction was then cooled to room temperature and concentrated under reduced pressure and purified by column chromatography on silica gel eluting with a gradient of 0-30% EtOAc in heptane to afford 5-chloro-4-(trimethylstannyl)isoquinoline (0.20 g, 0.60 mmol, 76% yield). m/z (ESI, +ve ion): 328.1 (M+H)⁺.

### 2-(8-Ethylnaphthalen-1-yl)-4,4,5,5-tetramethyl-1,3,2-dioxaborolane (Intermediate K

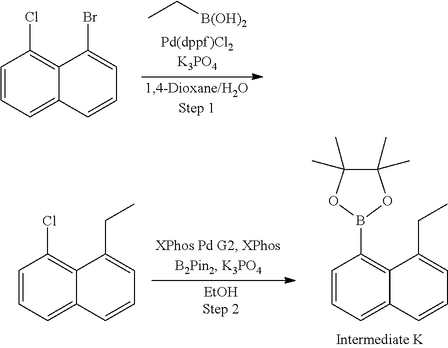

Step 1: 1-Chloro-8-ethylnaphthalene. To a 10 ml pressure vial was added 1-bromo-8-chloronaphthalene (3.5 g, 14.49 mmol, Ambeed), ethylboronic acid (3.21 g, 43.5 mmol, Combi-Blocks Inc.), potassium phosphate tribasic monohydrate (8.34 g, 36.2 mmol, Sigma-Aldrich Corporation) and dichloro-1,1′-bis(diphenylphosphino)ferrocene palladium(II) dichloromethane (0.592 g, 0.725 mmol, Strem Chemicals, Inc.) in 1,4-dioxane (46 mL) and water (2.3 mL) solvent mixture (0.3 M). The mixture was stirred at 70° C. for 2 h. The reaction mixture was concentrated under reduced pressure to afford the crude product. The crude product was isolated and purified by column chromatography on silica gel, eluting with heptane to yield 1-chloro-8-ethylnaphthalene (2.4 g, 12.59 mmol, 87% yield) as a yellow liquid. ¹H NMR (400 MHz, CHLOROFORM-d) δ ppm 7.75-7.78 (m, 1H), 7.69-7.75 (m, 1H), 7.56-7.59 (m, 1H), 7.37-7.44 (m, 2H), 7.29-7.35 (m, 1H), 3.49-3.57 (m, 2H), 1.35-1.42 (m, 3H).

Step 2: 2-(8-Ethylnaphthalen-1-yl)-4,4,5,5-tetramethyl-1,3,2-dioxaborolane. A mixture of 1-chloro-8-ethylnaphthalene (1.77 g, 9.28 mmol), bis(pinacalato)diboron (2.83 g, 11.14 mmol, Sigma-Aldrich Corporation), potassium phosphate tribasic (5.91 g, 27.8 mmol, Sigma-Aldrich Corporation), chloro(2-dicyclohexylphosphino-2′,4′,6′-triisopropyl-1,1′-biphenyl)[2-(2′-amino-1,1′-biphenyl)]palladium(II) (0.146 g, 0.186 mmol, Sigma-Aldrich Corporation), and 2-dicyclohexylphosphino-2,4,6,-tri-i-propyl-1,1-biphenyl (0.044 g, 0.093 mmol, Sigma-Aldrich Corporation) in ethanol (0.275 M) contained in pressure release vial was purged with nitrogen, capped and stirred to 23° C. for 18 h. Upon completion, the resulting solution was filtered, and concentrated under reduced pressure to afford the crude product. The resulting crude was purified by column chromatography on silica gel, eluting with 5% ethyl acetate in heptane to afford 2-(8-ethylnaphthalen-1-yl)-4,4,5,5-tetramethyl-1,3,2-dioxaborolane (1.23 g, 4.36 mmol, 47% yield)) as a white solid. m/z (ESI, +ve ion): 283.2. ¹H NMR (400 MHz, CHLOROFORM-d) δ ppm 7.87 (dd, J=8.2, 1.5 Hz, 1H), 7.60-7.74 (m, 2H), 7.35-7.49 (m, 3H), 3.24 (q, J=7.5 Hz, 2H), 1.43-1.48 (m, 12H), 1.36-1.41 (m, 3H).

### 2-(8-Ethyl-7-fluoronaphthalen-1-yl)-4,4,5,5-tetramethyl-1,3,2-dioxaborolane (Intermediate L

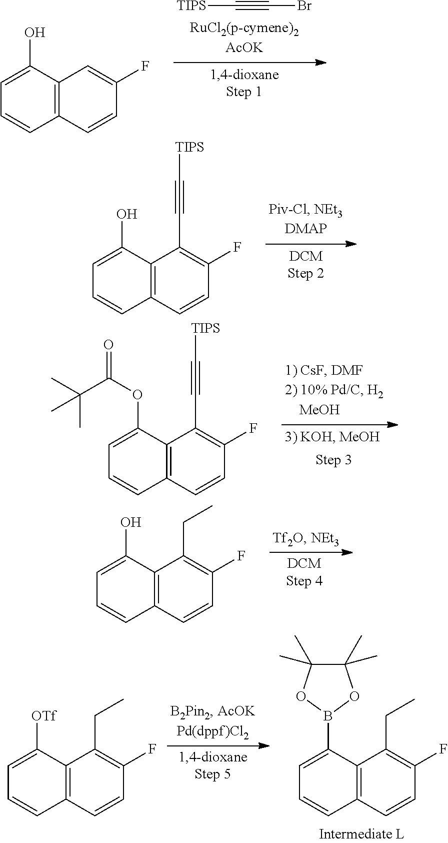

Step 1: 7-Fluoro-8-((triisopropylsilyl)ethynyl)naphthalen-1-ol. A pressure relief vial was charged with potassium acetate (1.21 g, 12.3 mmol, Sigma Aldrich), 7-fluoro-1-naphthol (1.00 g, 6.17 mmol, Enamine), dichloro(p-cymene)ruthenium(II)dimer (0.378 g, 0.617 mmol, Alfa Aesar) and then purged with nitrogen gas for 5 min. The solids were then suspended in 1,4-dioxane (12 mL) and (bromoethynyl)triisopropylsilane (1.77 g, 1.63 mL, 6.78 mmol, Enamine) was added. The reaction was then stirred at 110° C. for 18 h and subsequently at room temperature for 2 days. Volatiles were removed in vacuo and the crude material was absorbed onto silica gel. The crude product was purified by column chromatography on silica gel, eluting with a gradient of 0-20% EtOAc in heptane to yield 7-fluoro-8-((triisopropylsilyl)ethynyl)naphthalen-1-ol (1.9 g, 5.55 mmol, 90% yield) as a yellow oil. m/z (ESI, +ve ion): 343.0 (M+H)⁺.

Step 2: 7-Fluoro-8-((triisopropylsilyl)ethynyl)naphthalen-1-yl pivalate. 7-Fluoro-8-((triisopropylsilyl)ethynyl)naphthalen-1-ol (1.00 g, 2.92 mmol) was dissolved in dichloromethane (11 mL) and cooled to 0° C. DMAP (0.07 g, 0.58 mmol, Sigma-Aldrich Corporation) and TEA (0.89 g, 1.23 mL, 8.76 mmol, Sigma-Aldrich Corporation) were added, followed by dropwise addition of pivaloyl chloride (1.06 g, 1.08 mL, 8.76 mmol, Sigma-Aldrich Corporation). The mixture was warmed to room temperature and stirred for 45 minutes. Water (10 mL) was added and the aqueous layer extracted with DCM (2×10 mL). The combined organic phase was dried over anhydrous Na₂SO₄. Volatiles were removed in vacuo and the crude material was purified by column chromatography on silica gel, eluting with a gradient of 0-10% EtOAc in heptane to yield 7-fluoro-8-((triisopropylsilyl)ethynyl)naphthalen-1-yl pivalate (1.40 g, 3.28 mmol, 112% yield) as a yellow crystalline solid. m/z (ESI, +ve ion): 427.4 (M+H)⁺.

Step 3: 8-Ethyl-7-fluoronaphthalen-1-ol. A scintillation vial was charged with 7-fluoro-8-((triisopropylsilyl)ethynyl)naphthalen-1-yl pivalate (1.13 g, 2.65 mmol) and dissolved in DMF (12 mL). Cesium fluoride (4.02 g, 26.5 mmol, Sigma-Aldrich Corporation) was added and the mixture stirred at room temperature for 30 minutes. Water (100 mL) was added and the aqueous phase extracted with EtOAc (2×20 mL). The combined organic layer was dried over Na₂SO₄ and volatiles were then removed in vacuo to yield 8-ethynyl-7-fluoronaphthalen-1-yl pivalate (0.716 g, 2.65 mmol, quant.) as a crude yellow oil that was used without further purification. 8-Ethynyl-7-fluoronaphthalen-1-yl pivalate (716 mg, 2.65 mmol) was dissolved in MeOH (9 mL) and palladium on activated carbon (85 mg, 0.795 mmol, Sigma-Aldrich Corporation) was added. The reaction vessel was purged with H₂ and then stirred under a H₂ atmosphere (15 psi) at room temperature for 2 h. The mixture was filtered over Celite, washed with EtOAc until the filtrate ran clear and volatiles were removed in vacuo. The crude material was then dissolved in MeOH (10 mL) and potassium hydroxide (446 mg, 7.95 mmol, VWR International, LLC) was added. After stirring at room temperature for 2 h, the pH of the solution was adjusted to pH=3 using 1 M aq. HCl. Water (20 mL) was added and the aqueous phase extracted with EtOAc (3×10 mL). The combined organic layer was dried over Na₂SO₄ and volatiles were removed in vacuo. The crude material was purified by column chromatography on silica gel, eluting with a gradient of 0-20% EtOAc in heptane to yield 8-ethyl-7-fluoronaphthalen-1-ol (350 mg, 1.84 mmol, 70% yield) over 3 steps as a yellow oil. m/z (ESI, +ve ion): 191.2 (M+H)⁺.

Step 4: 8-Ethyl-7-fluoronaphthalen-1-yl trifluoromethanesulfonate. 8-Ethyl-7-fluoronaphthalen-1-ol (350 mg, 1.84 mmol) was dissolved in DCM and cooled to 0° C. TEA (279 mg, 0.388 mL, 2.76 mmol, Sigma-Aldrich Corporation) was added, followed by dropwise addition of a 1 M Tf₂O solution (2.02 mL, 2.02 mmol, Sigma-Aldrich Corporation). The mixture was stirred at room temperature for 20 minutes and poured into ice water (20 mL). The aqueous phase as extracted with DCM (2×10 mL), the combined organic layers were dried over Na₂SO₄ and volatiles were removed in vacuo. The crude mixture was purified by column chromatography on silica gel, eluting with a gradient of 0-5% EtOAc in heptane to yield 8-ethyl-7-fluoronaphthalen-1-yl trifluoromethanesulfonate (474 mg, 1.47 mmol, 80% yield) as a colourless oil. ¹H NMR (400 MHz, CHLOROFORM-d) δ ppm 7.86 (dd, J=8.3, 0.9 Hz, 1H), 7.79 (dd, J=9.4, 6.5 Hz, 1H), 7.59 (dt, J=7.7, 0.8 Hz, 1H), 7.44 (t, J=8.2 Hz, 1H), 7.36 (t, J=9.4 Hz, 1H), 3.32 (qd, J=7.5, 2.9 Hz, 2H), 1.29 (t, J=7.4 Hz, 3H).

Step E: 2-(8-Ethyl-7-fluoronaphthalen-1-yl)-4,4,5,5-tetramethyl-1,3,2-dioxaborolane. Potassium acetate (429 mg, 4.38 mmol, Sigma-Aldrich Corporation) was placed in a pressure relief vial and dried under vacuum. Then, 8-ethyl-7-fluoronaphthalen-1-yl trifluoromethanesulfonate (470 mg, 1.46 mmol), bis(pinacalato)diboron (741 mg, 2.92 mmol, Combi-Blocks Inc.) and [1,1′-bis(diphenylphosphino)ferrocene]dichloropalladium(II) (107 mg, 0.15 mmol, Sigma-Aldrich Corporation) were added and the mixture stirred at 90° C. for 3 h and then at room temperature for 12 h. Volatiles were removed in vacuo and the crude mixture was purified by column chromatography on silica gel, eluting with a gradient of 0-15% EtOAc in heptane to yield 2-(8-ethyl-7-fluoronaphthalen-1-yl)-4,4,5,5-tetramethyl-1,3,2-dioxaborolane (150 mg, 0.50 mmol, 34% yield) as a yellow wax. m/z (ESI, +ve ion): 301.0 (M+H)⁺.

### ((2-Fluoro-8-(4,4,5,5-tetramethyl-1,3,2-dioxaborolan-2-yl)naphthalen-1-yl)ethynyl)triisopropylsilane (Intermediate M

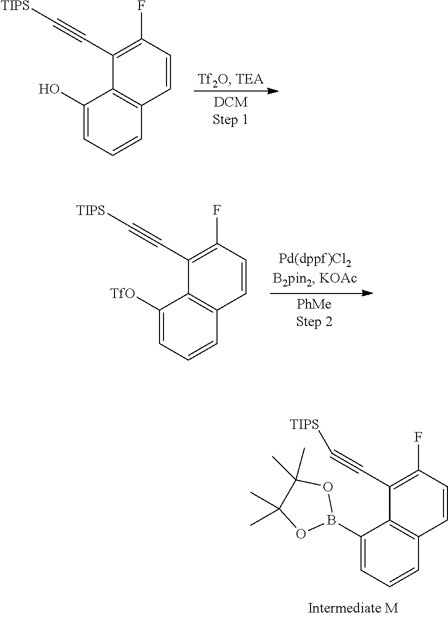

Step 1: 7-Fluoro-8-((triisopropylsilyl)ethynyl)naphthalen-1-yl trifluoromethanesulfonate. To a 25-mL round-bottomed flask was added 7-fluoro-8-((triisopropylsilyl)ethynyl)naphthalen-1-ol (1.1 g, 3.21 mmol), TEA (0.487 g, 0.677 mL, 4.82 mmol), and DCM (32 mL). the solution was cooled to 0° C. and Tf₂O(1 M in DCM, 3.53 mL, 3.53 mmol) was added. The cooling bath was removed and the reaction stirred at room temperature for 45 minutes. Upon completion, reaction was washed with satd NaHCO₃. The organics were dried over sodium sulfate and concentrated under reduced pressure to give 7-fluoro-8-((triisopropylsilyl)ethynyl)naphthalen-1-yl trifluoromethanesulfonate (2.01 g, 4.24 mmol, 132% yield) as a dark red oil used in the following step as is. ¹H NMR (400 MHz, CHLOROFORM-d): δ ppm 7.82-7.89 (m, 2H), 7.58 (d, J=7.9 Hz, 1H), 7.45-7.52 (m, 1H), 7.40 (t, J=8.7 Hz, 1H), 1.17-1.25 (m, 21H).

Step 2: ((2-Fluoro-8-(4,4,5,5-tetramethyl-1,3,2-dioxaborolan-2-yl)naphthalen-1-yl)ethynyl)triisopropylsilane. A 250-mL round bottom flask was charged with 7-fluoro-8-((triisopropylsilyl)ethynyl)naphthalen-1-yl trifluoromethanesulfonate (2.01 g, 4.24 mmol), bis(pinacalato)diboron (2.15 g, 8.47 mmol), KOAc (1.46 g, 14.82 mmol) and 1,1′-bis(diphenylphosphino)ferrocene-palladium dichloride (0.620 g, 0.847 mmol) in toluene (42 mL). The reaction was purged with N₂ for 5 minutes and then stirred at 80° C. for 15 h. Upon completion, the reaction was cooled to room temperature and concentrated under reduced pressure. The crude product was purified by column chromatographed on silica gel, eluting with a gradient of 0-40% EtOAc in heptane to provide ((2-fluoro-8-(4,4,5,5-tetramethyl-1,3,2-dioxaborolan-2-yl)naphthalen-1-yl)ethynyl)triisopropylsilane (783 mg, 1.730 mmol, 41% yield) as an orange solid. m/z (ESI, +ve ion): 453.2 (M+H)⁺.

### 5-Chloro-4-(4,4,5,5-tetramethyl-1,3,2-dioxaborolan-2-yl)naphthalen-2-ol (Intermediate N

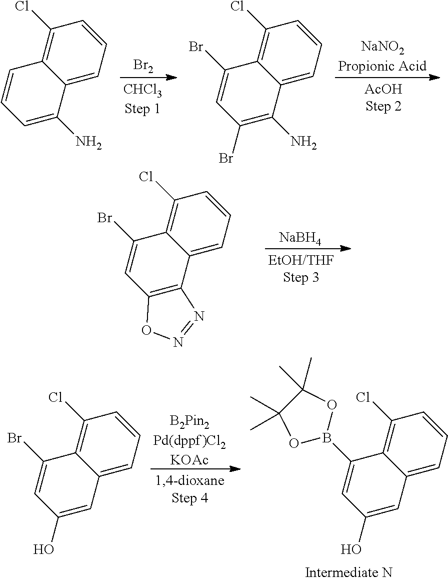

Step 1: 2,4-Dibromo-5-chloronaphthalen-1-amine. To a stirring solution of 5-chloronaphthalen-1-amine (2.0 g, 11.26 mmol, Combi-Blocks Inc.) in chloroform (56.3 mL) was added bromine (3.60 g, 1.154 mL, 22.52 mmol) in chloroform (56.3 mL) dropwise. After the addition was complete the reaction mixture was then heated to 50° C. After 3 h, additional bromine (1.80 g, 0.577 mL, 11.26 mmol) in chloroform (28 mL) was added dropwise. After stirring overnight, the reaction was cooled to room temperature and concentrated under reduced pressure. Water (100 mL) and EtOAc (100 mL) was added and the reaction was transferred to a separatory funnel. The layers were separated and the aqueous layer was extracted with EtOAc (3×100 mL). The organic layers were combined, dried over magnesium sulfate, filtered, and concentrated under reduced pressure. The resultant crude reaction mixture was purified by column chromatography on silica gel eluting with a gradient of 0-25% EtOAc in heptane to provide 2,4-dibromo-5-chloronaphthalen-1-amine (2.46 g, 7.34 mmol, 65% yield) as a dark purple solid. m/z (ESI, +ve ion): 383.8 (M+H)⁺.

Step 2: 5-Bromo-6-chloronaphtho[1,2-d][1,2,3]oxadiazole. To a cooled stirring solution of 2,4-dibromo-5-chloronaphthalen-1-amine (500 mg, 1.491 mmol) in acetic acid (12.218 mL) and propionic acid (1104 mg, 1.115 mL, 14.91 mmol) was added sodium nitrite (154 mg, 2.236 mmol) at 0° C. The reaction was stirred at this temperature for 30 minutes, and then was warmed to room temperature. After 1 h, the reaction was diluted with water (25 mL) and EtOAc (25 mL). The reaction was transferred to a separatory funnel and the layers were separated. The aqueous layer was then extracted with EtOAc (3×25 mL) and the combined organic layers were dried over magnesium sulfate, filtered, and concentrated under reduced pressure. The resultant crude solid was carried forward without further purification. m/z (ESI, +ve ion): 283.0 (M+H)⁺.

Step 3: 4-Bromo-5-chloronaphthalen-2-ol. A 150 mL round bottom flask was charged with 5-bromo-6-chloronaphtho[1,2-d][1,2,3]oxadiazole (423 mg, 1.492 mmol) and the solid was dissolved in ethanol (22.606 mL) and tetrahydrofuran (22.61 mL). The mixture was then cooled to 0° C. and sodium borohydride (130 mg, 3.43 mmol) was added. The reaction was allowed to slowly warm to room temperature over 1.5 h, and then stirred at room temperature. After an additional 2 h, the reaction was concentrated under reduced pressure and then water (25 mL), 1 N HCl (25 mL), and EtOAc (50 mL) was added. The mixture was transferred to a separatory funnel, and the layers were separated. The aqueous layer was extracted with EtOAc (3×50 mL) and the combined organic layers were dried over magnesium sulfate, filtered, and concentrated under reduced pressure. The resultant crude oil was then purified by column chromatography eluting with a gradient of 0-25% EtOAc in heptane to provide 4-bromo-5-chloronaphthalen-2-ol (128 mg, 0.497 mmol, 33.3% yield over two steps). m/z (ESI, +ve ion): 256.7 (M+H)⁺.

Step 4: 5-Chloro-4-(4,4,5,5-tetramethyl-1,3,2-dioxaborolan-2-yl)naphthalen-2-ol. A 20-mL vial was charged with 4-bromo-5-chloronaphthalen-2-ol (128 mg, 0.497 mmol), [1,1′-bis(diphenylphosphino)ferrocene]dichloropalladium(II) (109 mg, 0.149 mmol), potassium acetate (146 mg, 1.491 mmol) and bis(pinacalato)diboron (189 mg, 0.746 mmol) in 1,4-dioxane (4971 μL). The reaction was purged with nitrogen for 5 minutes and then stirred at 80° C. After 2 h the reaction was cooled to room temperature to afford a crude black oil. The crude material was absorbed onto a plug of silica gel and purified by column chromatography on silica gel, eluting with a gradient of 0-30% EtOAc in heptane, to provide 5-chloro-4-(4,4,5,5-tetramethyl-1,3,2-dioxaborolan-2-yl)naphthalen-2-ol (73.2 mg, 0.240 mmol, 48% yield) as a pink solid. m/z (ESI, +ve ion): 305.2 (M+H)⁺. ¹H NMR (400 MHz, DMSO-d₆) δ ppm 9.96 (s, 1H) 7.71 (dd, J=8.15, 1.25 Hz, 1H) 7.34-7.44 (m, 2H) 7.20-7.23 (m, 1H) 7.15-7.19 (m, 1H) 1.36 (s, 12H).

### ((5,6-Difluoro-4-(4,4,5,5-tetramethyl-1,3,2-dioxaborolan-2-yl)naphthalen-2-yl)oxy)triisopropylsilane (Intermediate O

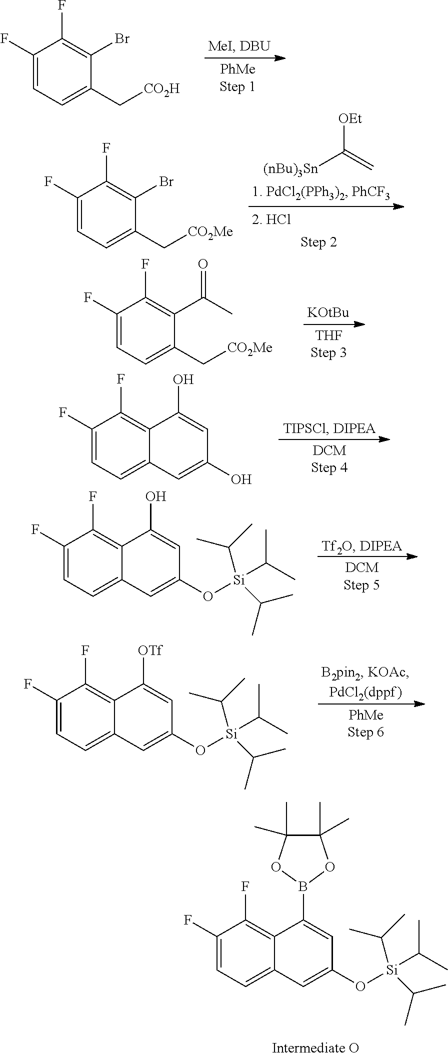

Step 1: Methyl 2-(2-bromo-3,4-difluorophenyl)acetate. To a 100-mL round-bottomed flask was added 2-(2-bromo-3,4-difluorophenyl)acetic acid (2.0 g, 7.97 mmol, AstaTech) and DBU (1.213 g, 1.201 mL, 7.97 mmol), and toluene (40 mL). To the mixture was added MeI (2.26 g, 0.996 mL, 15.93 mmol) and the mixture was stirred at rt for 4 h. Upon completion, the reaction was diluted with water and extracted with EtOAc. The organic layers were combined, dried, concentrated, and chromatographed with 0-50% EtOAc in heptane to give methyl 2-(2-bromo-3,4-difluorophenyl)acetate (1.62 g, 6.11 mmol, 77% yield) as a colorless oil. m/z (ESI): 264.96 (M+H)⁺.

Step 2: Methyl 2-(2-acetyl-3,4-difluorophenyl)acetate. A microwave vial was charged with methyl 2-(2-bromo-3,4-difluorophenyl)acetate (1.62 g, 6.11 mmol), trifluorotoluene (15 mL), tributyl(1-ethoxyvinyl)stannane (4.41 g, 12.22 mmol, Synthonix Inc.), and trans-dichlorobis(triphenyl-phosphine)palladium (ii) (0.858 g, 1.222 mmol, Strem Chemicals, Inc.). The vial was purged with nitrogen for 2 min, sealed, and placed in a microwave reactor for 12 h at 150° C. Upon completion, the mixture was filtered through a celite/silica plug and concentrated. The resulting yellow oil was dissolved in THF (10 mL) and 5 mL of 5 N HCl were added. The reaction was stirred at rt for 30 min. Upon completion, the reaction was poured slowly into a separatory funnel containing saturated NaHCO₃. Mixture was extracted with EtOAc, dried, concentrated and chromatographed with 0-30% EtOAc in heptane to give methyl 2-(2-acetyl-3,4-difluorophenyl)acetate (1.21 g, 5.30 mmol, 87% yield) as a colorless oil. m/z (ESI): 229.07(M+H)⁺.

Step 3: 7,8-Difluoronaphthalene-1,3-diol. To a 250 mL round-bottomed flask was added methyl 2-(2-acetyl-3,4-difluorophenyl)acetate (1.21 g, 5.30 mmol), KOtBu (1.785 g, 15.91 mmol) and THF (40 mL). The flask was capped and placed in a preheated aluminum block kept at 80° C. for 3 h. Upon completion, the reaction was diluted with 1 M HCl, extracted with DCM, and dried to give 7,8-difluoronaphthalene-1,3-diol (1.04 g, 5.30 mmol, 100% yield) as a red oil carried forward as is. m/z (ESI): 197.04 (M+H)⁺.

Step 4: 7,8-Difluoro-3-((triisopropylsilyl)oxy)naphthalen-1-ol. To a 100-mL round-bottomed flask was added 7,8-difluoronaphthalene-1,3-diol (750 mg, 3.82 mmol), DIPEA (2.00 mL, 11.47 mmol) and DCM (38 mL). The solution was cooled to 0° C. and TIPS-C1 (663 mg, 0.729 mL, 3.44 mmol) was added. The reaction was allowed to warm to rt. Upon completion, the reaction was concentrated and chromatographed with 0-30% EtOAc in heptanes. Isomers separated on a silica gel column with 0-30% EtOAc in heptane to give 7,8-difluoro-3-((triisopropylsilyl)oxy)naphthalen-1-ol (883 mg, 2.505 mmol, 65.5% yield) (first eluting isomer) as the major product and 5,6-difluoro-4-((triisopropylsilyl)oxy)naphthalen-2-ol (175 mg, 0.496 mmol, 12.98% yield) (second eluting isomer) as the minor product. m/z (ESI): 353.17 (M+H)⁺.

Step 5: 7,8-Difluoro-3-((triisopropylsilyl)oxy)naphthalen-1-yl trifluoromethanesulfonate. To a 100-mL round-bottomed flask was added 7,8-difluoro-3-((triisopropylsilyl)oxy)naphthalen-1-ol (883 mg, 2.50 mmol), DIPEA (1.31 mL, 7.51 mmol) and DCM (25 mL). The solution was cooled to 0° C. and Tf₂O (1 M in DCM, 2.76 mL, 2.76 mmol) was added. The reaction was allowed to warm to rt and stir for 1 h. Upon completion, the reaction was poured into a separatory funnel containing saturated NaHCO₃ and extracted with DCM. The organics were dried and concentrated to give 7,8-difluoro-3-((triisopropylsilyl)oxy)naphthalen-1-yl trifluoromethanesulfonate (1214 mg, 2.505 mmol, 100% yield) as an orange oil used in the following step as is. m/z (ESI): 485.12 (M+H)⁺.

Step 6: ((5,6-Difluoro-4-(4,4,5,5-tetramethyl-1,3,2-dioxaborolan-2-yl)naphthalen-2-yl)oxy)triisopropylsilane. A 100-mL round-bottomed flask was charged with 7,8-difluoro-3-((triisopropylsilyl)oxy)naphthalen-1-yl trifluoromethanesulfonate (1.21 g, 2.497 mmol), bis(pinacalato)diboron (1.268 g, 4.99 mmol), potassium acetate (0.858 g, 8.74 mmol), 1,1′-bis(diphenylphosphino)ferrocene-palladium dichloride (0.365 g, 0.499 mmol) and toluene (25 mL). The reaction was purged with nitrogen for 5 minutes and then stirred at 80° C. for 12 h. Upon completion, the reaction was concentrated and chromatographed with a gradient of 0-40% EtOAc in hexanes to give ((5,6-difluoro-4-(4,4,5,5-tetramethyl-1,3,2-dioxaborolan-2-yl)naphthalen-2-yl)oxy)triisopropylsilane (966 mg, 2.089 mmol, 84% yield). m/z (ESI): 463.26 (M+H)⁺.

### 2-(8-Ethyl-7-fluoro-3-(methoxymethoxy)naphthalen-1-yl)-4,4,5,5-tetramethyl-1,3,2-dioxaborolane (Intermediate P

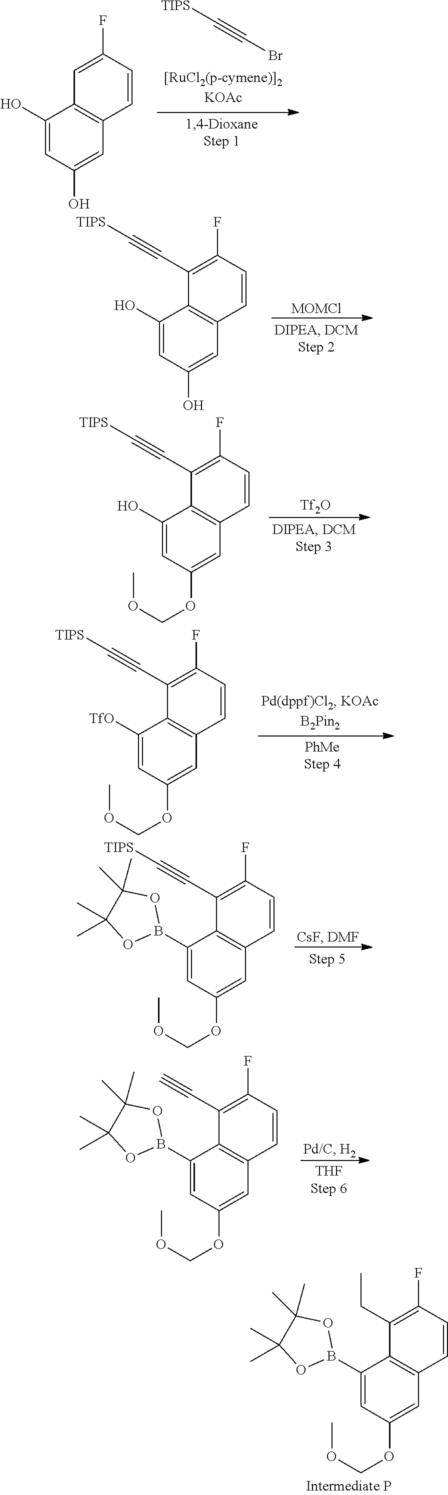

Step 1: 7-Fluoro-8-((triisopropylsilyl)ethynyl)naphthalene-1,3-diol. To a mixture of 7-fluoronaphthalene-1,3-diol (3.30 g, 18.52 mmol) and 2-bromoethynyl(triisopropyl)silane (5.81 g, 22.23 mmol) in dioxane (60 mL) was added KOAc (3.64 g, 37.05 mmol) and dichloro(p-cymene)ruthenium(II) dimer (1.13 g, 1.85 mmol) in one portion at 15° C. under N₂. The mixture was stirred at 110° C. for 2 h. The mixture was cooled to 15° C. and poured into ice-water (w/w=1/1, 60 mL). The mixture was extracted with ethyl acetate (50 mL×3). The combined organic phases were dried with anhydrous Na₂SO₄, filtered and concentrated in vacuo. The residue was purified by column chromatography on silica gel, eluting with petroleum ether/ethyl acetate=8/1, 1/1 to give the product (4.30 g, 11.27 mmol, 65% yield) as yellow solid. m/z (ESI): 359.2 (M+H)⁺.

Step 2: 7-Fluoro-3-(methoxymethoxy)-8-((triisopropylsilyl)ethynyl)naphthalen-1-ol. To a mixture of 7-fluoro-8-((triisopropylsilyl)ethynyl)naphthalene-1,3-diol (4.30 g, 11.99 mmol) in DCM (60 mL) was added DIPEA (4.65 g, 35.98 mmol, 6.27 mL). Then MOMCl (1.16 g, 14.39 mmol, 1.09 mL) was added in portions at 0° C. under N₂. The mixture was stirred at 15° C. for 12 h. The mixture was poured into ice-water (w/w=1/1) (60 mL) and stirred for 20 min. The mixture was extracted with ethyl acetate (60 mL×3). The combined organic phase was washed with brine (60 mL×2), dried with anhydrous Na₂SO₄, filtered and concentrated in vacuo. The residue was purified by column chromatography on silica gel, eluting with petroleum ether/ethyl acetate 4/1 to 1/1 to give 7-fluoro-3-(methoxymethoxy)-8-(2-triisopropylsilylethynyl)naphthalen-1-ol (2.00 g, 4.97 mmol, 41% yield) as a brown solid.

Step 3: 7-Fluoro-3-(methoxymethoxy)-8-((triisopropylsilyl)ethynyl)naphthalen-1-yl trifluoromethanesulfonate. To a mixture of 7-fluoro-3-(methoxymethoxy)-8-(2-triisopropylsilylethynyl)naphthalen-1-ol (2.5 g, 6.21 mmol) in DCM (30 mL) was added DIPEA (2.41 g, 18.63 mmol, 3.25 mL) in one portion at 15° C. under N₂. Then to the mixture was added Tf₂O (2.63 g, 9.32 mmol, 1.54 mL) in portions at −40° C. under N₂. The mixture was stirred at −40° C. for 1 h. The mixture was poured into ice-water (w/w=1/1, 30 mL). The aqueous phase was extracted with dichloromethane (30 mL×3). The combined organic phase was dried with anhydrous Na₂SO₄, filtered and concentrated in vacuo. The residue was purified by column chromatography on silica gel, eluting with petroleum ether/ethyl acetate 40/1 to 8/1 to give 7-fluoro-3-(methoxymethoxy)-8-((triisopropylsilyl)ethynyl)naphthalen-1-yl trifluoromethanesulfonate (2.50 g, 4.68 mmol, 75% yield) as yellow oil.

Step 4: ((2-Fluoro-6-(methoxymethoxy)-8-(4,4,5,5-tetramethyl-1,3,2-dioxaborolan-2-yl)naphthalen-1-yl)ethynyl)triisopropylsilane. To a mixture of 7-fluoro-3-(methoxymethoxy)-8-((triisopropylsilyl)ethynyl)naphthalen-1-yl trifluoromethanesulfonate (2.50 g, 4.68 mmol) in toluene (25 mL) was added KOAc (1.38 g, 14.03 mmol), bis(pinacalato)diboron (2.37 g, 9.35 mmol) and Pd(dppf)Cl₂ (0.34 g, 0.47 mmol) in one portion at 15° C. under N₂. The mixture was stirred at 130° C. for 3 h. The mixture was cooled to 15° C. and concentrated under reduced pressure at 45° C. The residue was poured into ice-water (w/w=1/1, 40 mL). The mixture was extracted with ethyl acetate (40 mL×3). The combined organic phase was dried with anhydrous Na₂SO₄, filtered and concentrated in vacuo. The residue was purified by column chromatography on silica gel, eluting with petroleum ether/ethyl acetate 40/1 to 10/1 to give ((2-fluoro-6-(methoxymethoxy)-8-(4,4,5,5-tetramethyl-1,3,2-dioxaborolan-2-yl)naphthalen-1-yl)ethynyl)triisopropylsilane (1.20 g, 2.34 mmol, 50% yield) as yellow solid. m/z (ESI): 513.2 (M+H)⁺.

Step 5: 2-(8-Ethynyl-7-fluoro-3-(methoxymethoxy)naphthalen-1-yl)-4,4,5,5-tetramethyl-1,3,2-dioxaborolane. To a mixture of ((2-fluoro-6-(methoxymethoxy)-8-(4,4,5,5-tetramethyl-1,3,2-dioxaborolan-2-yl)naphthalen-1-yl)ethynyl)triisopropylsilane (1.1 g, 2.15 mmol) in DMF (20 mL) was added CsF (1.96 g, 12.88 mmol, 0.47 mL) in one portion at 15° C. under N₂. The reaction mixture was stirred at 50° C. for 2 h. After 2 h, the mixture was poured into water (20 mL). The resulting aqueous mixture was extracted with ethyl acetate (20 mL×3). The combined organic phase was washed with brine (30 mL×2), dried with anhydrous Na₂SO₄, filtered and concentrated in vacuo to give the crude 2-(8-ethynyl-7-fluoro-3-(methoxymethoxy)naphthalen-1-yl)-4,4,5,5-tetramethyl-1,3,2-dioxaborolane (1.2 g, crude) as brown oil.

Step 6: 2-(8-Ethyl-7-fluoro-3-(methoxymethoxy)naphthalen-1-yl)-4,4,5,5-tetramethyl-1,3,2-dioxaborolane. To a solution of 2-(8-ethynyl-7-fluoro-3-(methoxymethoxy)naphthalen-1-yl)-4,4,5,5-tetramethyl-1,3,2-dioxaborolane (1.20 g, 3.37 mmol) in THF (10 mL) was added Pd—C(10/6, 20 mg) under Ar. The suspension was degassed under vacuum and purged with H₂ several times. The mixture was stirred under H₂ (15 psi) at 15° C. for 1 h. The reaction mixture was filtered and the filter cake was washed with THF (10 mL×3). The combined filtrate was concentrated under reduced pressure. The crude product was purified by column chromatography on silica gel, eluting with petroleum ether/ethyl acetate 25/1 to 10/1 to give 2-(8-ethyl-7-fluoro-3-(methoxymethoxy)naphthalen-1-yl)-4,4,5,5-tetramethyl-1,3,2-dioxaborolane (0.62 g, 1.69 mmol, 75% yield over two steps) as a yellow solid. m/z (ESI): 361.1 (M+H)⁺.

### tert-Butyl (1S,)-1-fluoro-3,8-diazabicyclo[3.2.1]octane-8-carboxylate (Intermediate Q1 and Q2

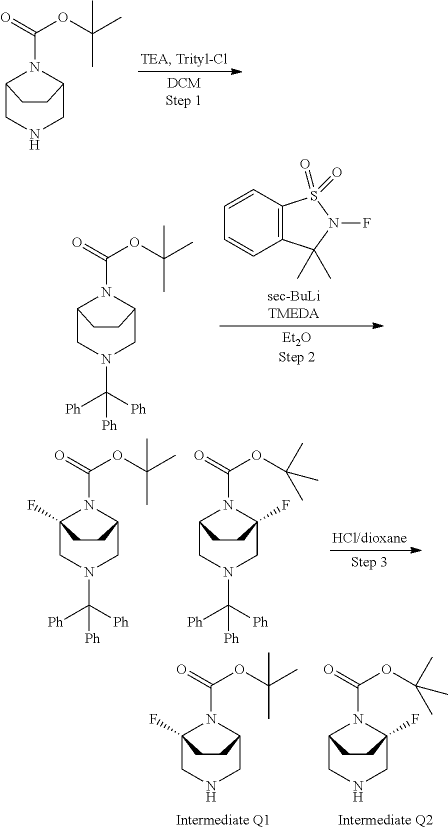

Step 1: A 40 mL vial was charged with tert-butyl 3,8-diazabicyclo[3.2.1]octane-8-carboxylate (1.00 g, 4.71 mmol, Pharmablock, Inc.) and (chlorodiphenylmethyl)benzene (1.4 g, 4.99 mmol, Enamine). The solids were then dissolved in dichloromethane (24 mL) and triethylamine (0.60 g, 0.8 mL, 5.65 mmol, Sigma-Aldrich Corporation) was added. The reaction was stirred for 2 d. The reaction was then concentrated and purified by column chromatography on silica gel, eluting with a gradient of 0-40% EtOAc in heptane to provide tert-butyl (1R,5S)-3-trityl-3,8-diazabicyclo[3.2.1]octane-8-carboxylate (2.00 g, 4.48 mmol, 95% yield) as white solid.

Step 2: To an oven dried vial, tert-butyl (1R,5S)-3-trityl-3,8-diazabicyclo[3.2.1]octane-8-carboxylate (0.35 g, 0.77 mmol) and N,N,N′,N′-tetramethylethylenediamine (0.20 g, 0.3 mL, 1.75 mmol, Sigma-Aldrich Corporation) was added and the solids were suspended in diethyl ether (7.7 mL). The reaction was cooled to −40° C. and sec-butyllithium solution (1.4 M in cyclohexane, 1.3 mL, 1.75 mmol, Sigma-Aldrich Corporation) was added dropwise. The reaction was slowly warmed to 0° C. and then stirred for 60 min. 2-Fluoro-3,3-dimethyl-2,3-dihydro-1,2-benzisothiazole 1,1-dioxide (0.41 g, 1.93 mmol, Sigma-Aldrich Corporation) in ether (1.5 mL) and 2-methyl THF (1.5 mL) was then added. The reaction was allowed to stir at this temperature for an additional 3 h. The reaction was quenched with saturated ammonium chloride (10 mL), and then was transferred to a separatory funnel. The layers were separated and the aqueous layer was extracted with DCM (3×10 mL). The combined organic layers were dried with sodium sulfate, filtered, and concentrated under reduced pressure. The crude solid was then purified by column chromatography on silica gel, eluting with a gradient of 0-25% EtOAc in heptane to provide white solid. The sample was purified by SFC using a (S,S) Whelk-O1 21×250, 5 micron, mobile phase of 15% methanol with triethylamine using a flowrate of 80 mL/min to generate 138 mg of peak 1 with ee of >95/a, and 139 mg of peak 2 with ee of >99%. Peak assignment determined by SFC with (S,S) Whelk-O1 and 15% methanol with 0.2% triethylamine. The two peaks were assigned tert-butyl (1S,5S)-1-fluoro-3-trityl-3,8-diazabicyclo[3.2.1]octane-8-carboxylate and tert-butyl (1R,5R)-1-fluoro-3-trityl-3,8-diazabicyclo[3.2.1]octane-8-carboxylate.

Step 3: A scintillation vial was charged with tert-butyl-1-fluoro-3-trityl-3,8-diazabicyclo[3.2.1]octane-8-carboxylate (92 mg, 0.20 mmol) and the solid was dissolved in 1,4-dioxane (1.9 mL). Then HCl in 1,4-dioxane (4 M, 0.1 mL, 0.58 mmol, Sigma-Aldrich Corporation) (diluted to 1 M) was added dropwise and the reaction was allowed to stir for 6 h. Sodium bicarbonate (49 mg, 0.58 mmol, Sigma-Aldrich Corporation) was then added and the reaction was stirred for an additional 30 min. The reaction was then concentrated under reduced pressure. The solid was then suspended in dichloromethane (5.0 mL) and passed through a short plug of celite. The organics were then concentrated to give the crude product, which used directly in the next step without further purification. m/z (ESI, +ve ion): 175.2 (M−^{t}Bu+H)⁺.

### 4,6-Dichloro-N,N-bis(4-methoxybenzyl)-5-(trifluoromethyl)pyridin-2-amine (Intermediate R

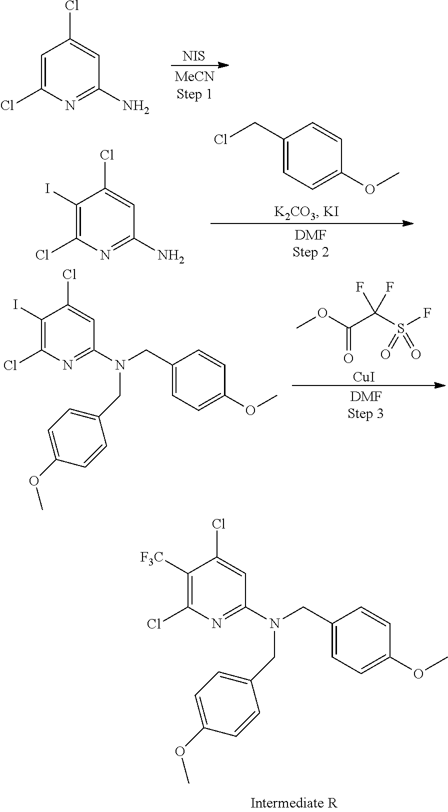

Step 1: 4,6-Dichloro-5-iodopyridin-2-amine. 2-Amino-4,6-dichloropyridine (2.0 g, 12.27 mmol, CombiBlocks) was dissolved in acetonitrile (24.5 mL) and NIS (3.6 g, 15.95 mmol, Oakwood Products, Inc.) was added. The mixture was stirred at 55° C. for 4 h. Saturated solution of sodium thiosulfate was added until the mixture discolored. MeCN was removed in vacuo and the aqueous layer was extracted with EtOAc (3×20 mL). Combined organic phases were dried over anhydrous Na₂SO₄. Volatiles were removed in vacuo and the residue was purified by column chromatography on silica gel, eluting with a gradient of 0-50% EtOAc in heptane to yield 4,6-dichloro-5-iodopyridin-2-amine (2.6 g, 9.00 mmol, 73% yield). m/z (ESI): 288.9 (M+H)⁺. ¹H NMR (400 MHz, DMSO-d6) δ ppm 6.73-6.78 (m, 2H), 6.60-6.62 (m, 1H).

Step 2: 4,6-Dichloro-5-iodo-N,N-bis(4-methoxybenzyl)pyridin-2-amine. 4,6-Dichloro-5-iodopyridin-2-amine (0.92 g, 3.18 mmol) was dissolved in N, N-dimethylformamide (6.4 mL), potassium carbonate (1.8 g, 12.74 mmol, Sigma-Aldrich Corporation), potassium iodide (0.26 g, 1.59 mmol, Sigma-Aldrich Corporation) and 4-methoxybenzyl chloride (1.6 g, 1.4 mL, 10.23 mmol, TCI America) was added and the mixture stirred at 110° C. for 8 h. After cooling to rt, water (20 mL) was added and the aqueous phase was extracted with EtOAc (2×15 mL). Combined organic phases were dried over anhydrous Na₂SO₄ and volatiles were removed in vacuo. The crude material was purified by column chromatography on silica gel, eluting with a gradient of 0-10% EtOAc in heptane to yield 4,6-dichloro-5-iodo-N,N-bis(4-methoxybenzyl)pyridin-2-amine (0.63 g, 1.19 mmol, 37% yield). m/z (ESI, +ve ion): 529.0 (M+H)⁺.

Step 3: 4,6-Dichloro-N,N-bis(4-methoxybenzyl)-5-(trifluoromethyl)pyridin-2-amine. Under nitrogen atmosphere, 4,6-dichloro-5-iodo-N,N-bis(4-methoxybenzyl)pyridin-2-amine (0.62 g, 1.17 mmol) was dissolved in N, N-dimethylformamide (5.8 mL), copper(I) iodide (0.33 g, 1.76 mmol, Strem Chemicals, Inc.) and 1,1-difluoro-2-methoxy-2-oxoethane-1-sulphonyl fluoride (0.34 g, 0.2 mL, 1.76 mmol, Sigma-Aldrich Corporation) were added and the mixture stirred at 100° C. for 4 h. The mixture was filtered through a syringe filter and purified via reverse phase HPLC to yield 4,6-dichloro-N,N-bis(4-methoxybenzyl)-5-(trifluoromethyl)pyridin-2-amine (0.43 g, 0.91 mmol, 78% yield) as white solid. ¹H NMR (400 MHz, CHLOROFORM-d) δ ppm 7.13-7.18 (m, 4H), 6.86-6.91 (m, 4H), 6.44-6.47 (m, 1H), 4.63-4.73 (m, 4H), 3.83 (s, 6H).

### 6-Bromo-N,N-bis(4-methoxybenzyl)-4-methyl-5-(trifluoromethyl)pyridin-2-amine (Intermediate S

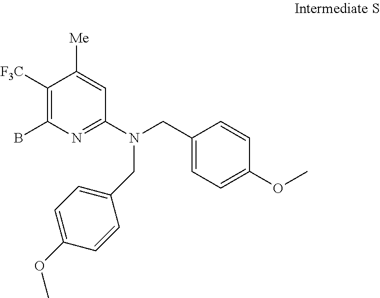

Synthesized in an analogous manner to Intermediate R, using 2-amino-4-methyl-6-bromopyridine (Ark Pharm, Inc.). m/z (ESI, +ve ion): 495.0 (M+H)⁺. ¹H NMR (400 MHz, CHLOROFORM-d) δ ppm 7.15-7.22 (m, 4H), 6.84-6.93 (m, 4H), 6.40-6.44 (m, 1H), 4.72-4.80 (m, 3H), 3.79-3.87 (m, 6H), 2.37-2.43 (m, 3H), 2.21-2.30 (m, 2H).

### Experimental Procedures

### 4-(4-((1R,5S)-3,8-Diazabicyclo[3.2.1]octan-8-yl)-8-fluoro-2-(((2R,7aS)-2-fluorotetrahydro-1H-pyrrolizin-7a(5H)-yl)methoxy)quinazolin-7-yl)naphthalen-2-ol

### Example 1

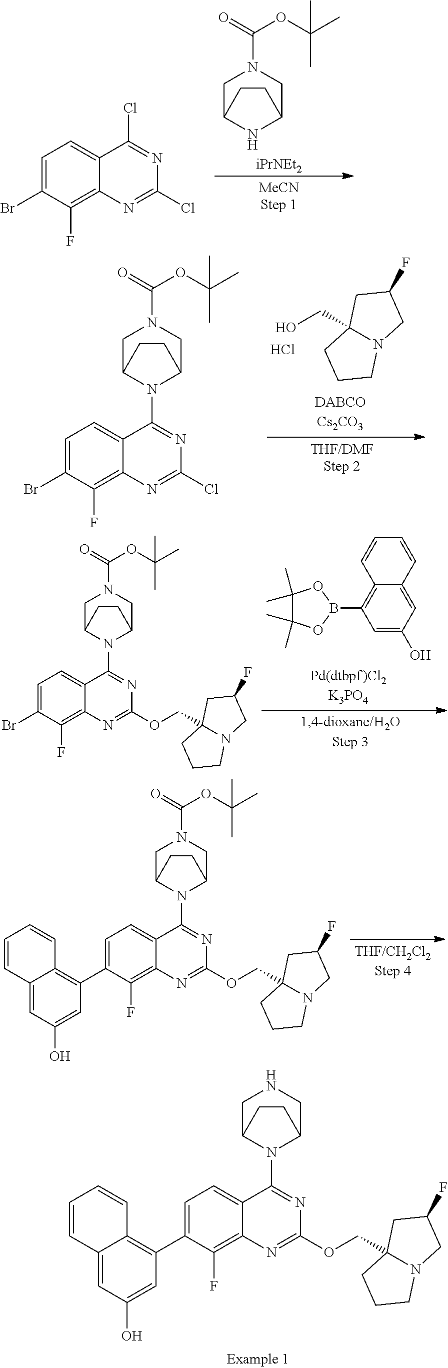

Step 1: tert-Butyl (1R,5S)-8-(7-bromo-2-chloro-8-fluoroquinazolin-4-yl)-3,8-diazabicyclo[3.2.1]octane-3-carboxylate. To a mixture of 7-bromo-2,4-dichloro-8-fluoroquinazoline (1.23 g, 4.15 mmol, Enamine) in acetonitrile (10.4 mL) was added N,N-diisopropylethylamine (0.56 g, 0.76 mL, 4.35 mmol, Sigma-Aldrich Corporation) and tert-butyl (1R,5S)-3,8-diazabicyclo[3.2.1]octane-3-carboxylate (0.90 g, 4.23 mmol, PharmaBlock). The reaction was stirred at room temperature for 1 h. The mixture was then filtered. The solid product was collected and dried under vacuum to yield tert-butyl (1R,5S)-8-(7-bromo-2-chloro-8-fluoroquinazolin-4-yl)-3,8-diazabicyclo[3.2.1]octane-3-carboxylate (1.46 g, 3.09 mmol, 75% yield). m/z (ESI, +ve ion): 471.0 (M+H)⁺.

Step 2: tert-butyl (1R,5S)-8-(7-bromo-8-fluoro-2-(((2R,7aS)-2-fluorotetrahydro-1H-pyrrolizin-7a(511)-yl)methoxy)quinazolin-4-yl)-3,8-diazabicyclo[3.2.1]octane-3-carboxylate. A 20-mL vial was charged with 1,4-diazabicyclo[2.2.2]octane (4.9 mg, 0.043 mmol, Sigma-Aldrich Corporation), cesium carbonate (0.85 g, 2.61 mmol, Sigma-Aldrich Corporation), ((2R,7aS)-2-fluorotetrahydro-1H-pyrrolizin-7a(5H)-yl)methanol hydrochloride (0.26 g, 1.30 mmol, AChemBlock), tert-butyl (1R,5S)-8-(7-bromo-2-chloro-8-fluoroquinazolin-4-yl)-3,8-diazabicyclo[3.2.1]octane-3-carboxylate (0.21 g, 0.44 mmol), N,N-dimethylformamide (0.72 mL) and tetrahydrofuran (1.45 mL). The reaction was stirred at 40° C. overnight. The crude product was purified by column chromatography on silica gel, eluting with 0-50% 3:1 EtOAc/EtOH in heptane with 2% triethylamine additive to yield tert-butyl (1R,5S)-8-(7-bromo-8-fluoro-2-(((2R,7aS)-2-fluorotetrahydro-1H-pyrrolizin-7a(5H)-yl)methoxy)quinazolin-4-yl)-3,8-diazabicyclo[3.2.1]octane-3-carboxylate (0.22 g, 0.37 mmol, 84% yield). m/z (ESI, +ve ion): 594.0 (M+H)⁺.

Step 3: tert-Butyl (1R,5S)-8-(8-fluoro-2-(((2R,7aS)-2-fluorotetrahydro-1H-pyrrolizin-7a(5H)-yl)methoxy)-7-(3-hydroxynaphthalen-1-yl)quinazolin-4-yl)-3,8-diazabicyclo[3.2.1]octane-3-carboxylate. An 8-mL vial was charged with [1,1′-bis(di-tert-butylphosphino)ferrocene]dichloropalladium(II) (11 mg, 0.017 mmol, Sigma-Aldrich Corporation), potassium phosphate tribasic (54 mg, 0.25 mmol, Acros Organics), 4-(4,4,5,5-tetramethyl-1,3,2-dioxaborolan-2-yl)-2-naphthol (34 mg, 0.13 mmol, Aurum Pharmatech LLC), tert-butyl (1R,5S)-8-(7-bromo-8-fluoro-2-(((2R,7aS)-2-fluorotetrahydro-1H-pyrrolizin-7a(5H)-yl)methoxy)quinazolin-4-yl)-3,8-diazabicyclo[3.2.1]octane-3-carboxylate (50 mg, 0.084 mmol), water (64.7 μL) and 1,4-dioxane (0.26 mL). The reaction was stirred at 90° C. for 1 h. After cooling, the crude mixture was purified by column chromatography on silica gel eluting with 0-50% 3:1 EtOAc/EtOH in heptane with 2% triethylamine additive to yield tert-butyl (1R,5S)-8-(8-fluoro-2-(((2R,7aS)-2-fluorotetrahydro-1H-pyrrolizin-7a(5H)-yl)methoxy)-7-(3-hydroxynaphthalen-1-yl)quinazolin-4-yl)-3,8-diazabicyclo[3.2.1]octane-3-carboxylate (37 mg, 0.057 mmol, 67% yield). m/z (ESI, +ve ion): 658.3 (M+H)⁺.

Step 4: 4-(4-((1R,5S)-3,8-Diazabicyclo[3.2.1]octan-8-yl)-8-fluoro-2-(((2R,7aS)-2-fluorotetrahydro-1H-pyrrolizin-7a(5H)-yl)methoxy)quinazolin-7-yl)naphthalen-2-ol. tert-Butyl (1R,5S)-8-(8-fluoro-2-(((2R,7aS)-2-fluorotetrahydro-1H-pyrrolizin-7a(5H)-yl)methoxy)-7-(3-hydroxynaphthalen-1-yl)quinazolin-4-yl)-3,8-diazabicyclo[3.2.1]octane-3-carboxylate was stirred in dichloromethane (0.26 mL) and trifluoroacetic acid (0.26 mL) at room temperature until completion. Solvents were removed under reduced pressure. The crude product was purified by reverse phase HPLC to yield 4-(4-((1R,5S)-3,8-diazabicyclo[3.2.1]octan-8-yl)-8-fluoro-2-(((2R,7aS)-2-fluorotetrahydro-1H-pyrrolizin-7a(5H)-yl)methoxy)quinazolin-7-yl)naphthalen-2-ol (25 mg, 0.045 mmol, 54% yield). m/z (ESI, +ve ion): 558.8 (M+H)⁺. ¹H NMR (400 MHz, METHANOL-d4) ppm 8.05 (d, J=0.6 Hz, 1H), 7.71-7.83 (m, 1H), 7.40-7.54 (m, 3H), 7.19-7.32 (m, 2H), 7.11-7.16 (m, 1H), 5.46-5.69 (2, 1H), 5.30 (br s, 2H), 4.74 (br s, 2H), 3.92 (br s, 3H), 3.65-3.78 (m, 2H), 3.41-3.54 (m, 3H), 2.56-2.80 (m, 2H), 2.28-2.52 (m, 5H), 2.09-2.26 (i, 3H).

TABLE 2
Additional Examples. Prepared in an Analogous Manner to Example 1, using
Intermediates A-P. Products were isolated as corresponding TFA salts.

| | | | MS | |
|---|---|---|---|---|
| | | | m/z | |
| | | | (ESI, | |
| Ex. | | | +ve | |
| # | Structure | Name | ion) | ¹H NMR |
| 2 | 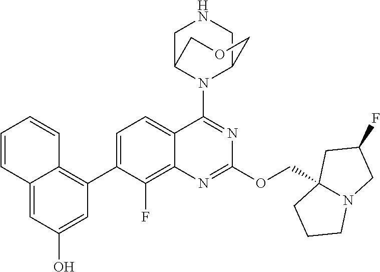 [SMILES: Oc1cc(-c2ccc3c(N4C5CNCC4COC5)nc(OC[C@@]45CCCN4C[C@H](F)C5)nc3c2F)c2ccccc2c1] | 4-(4-((1R,5S)-3- oxa-7,9- diazabicyclo[3.3. 1]nonan-9-yl)-8- fluoro-2- (((2R,7aS)-2- fluorotetrahydro- 1H-pyrrolizin- 7a(5H)- yl)methoxy) quinazolin-7- yl)naphthalen-2-ol | 574.9 (M + H)⁺ | ¹H NMR (400 MHz, METHANOL- d4) δ ppm 7.84 − 7.92 (m, 1 H), 7.73 − 7.82 (m, 1 H), 7.40 − 7.52 (m, 3 H), 7.19 − 7.32 (m, 2 H), 7.08 − 7.15 (m, 1 H), 5.45 − 5.74 (m, 1 H), 4.88 − 4.92 (m, 2 H), 4.68 − 4.78 (m, 2 H), 4.22 − 4.38 (m, 4 H), 3.70 − 4.13 (m, 7 H), 3.41 − 3.61 (m, 1 H), 2.56 − 2.83 (m, 2 H), 2.30 − 2.52 (m, 3 H), 2.11 − 2.27 (m, 1 H). |
| 3 | 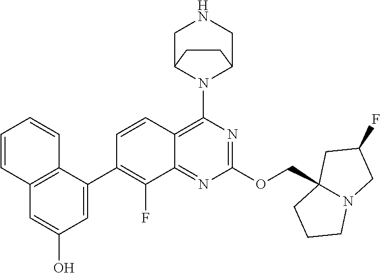 [SMILES: Oc1cc(-c2ccc3c(N4C5CCC4CNC5)nc(OC[C@]45CCCN4C[C@H](F)C5)nc3c2F)c2ccccc2c1] | 4-(4-((1R,5S)- 3,8- diazabicyclo[3.2. 1]octan-8-yl)-8- fluoro-2- (((2R,7aR)-2- fluorotetrahydro- 1H-pyrrolizin- 7a(5H)- yl)methoxy) quinazolin-7- yl)naphthalen-2- ol | 558.3 (M + H)⁺ | ¹H NMR (400 MHz, METHANOL- d4) δ ppm 8.01 − 8.10 (m, 1 H), 7.74 − 7.82 (m, 1 H), 7.41 − 7.55 (m, 3 H), 7.20 − 7.30 (m, 2 H), 7.09 − 7.17 (m, 1 H), 5.43 − 5.64 (m, 1 H), 5.20 − 5.35 (m, 2 H), 4.75 − 4.82 (m, 2 H), 3.97 − 4.14 (m, 1 H), 3.63 − 3.76 (m, 3 H), 3.43 − 3.61 (m, 4 H), 2.67 − 2.81 (m, 1 H), 2.40 − 2.58 (m, 2 H), 2.24 − 2.39 (m, 4 H), 2.10 − 2.22 (m, 3 H). |
| 4 | 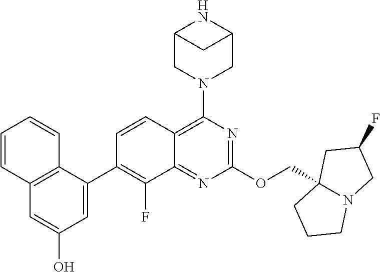 [SMILES: Oc1cc(-c2ccc3c(N4CC5CC(C4)N5)nc(OC[C@@]45CCCN4C[C@H](F)C5)nc3c2F)c2ccccc2c1] | 4-(4-((1R,5S)- 3,6- diazabicyclo[3.1. 1]heptan-3-yl)-8- fluoro-2- (((2R,7aS)-2- fluorotetrahydro- 1H-pyrrolizin- 7a(5H)- yl)methoxy) quinazolin-7- yl)naphthalen-2-ol | 544.6 (M + H)⁺ | ¹H NMR (400 MHz, METHANOL- d₄) δ ppm 8.41 (d, J = 8.6 Hz, 1 H), 7.73 − 7.82 (m, 1 H), 7.41 − 7.54 (m, 3 H), 7.27 − 7.31 (m, 1 H), 7.20 − 7.26 (m, 1 H), 7.11 − 7.18 (m, 1 H), 5.48 − 5.71 (m, 1 H), 4.75 − 4.82 (m, 2 H), 4.62 − 4.74 (m, 4 H), 3.80 − 4.13 (m, 3 H), 3.43 − 3.54 (m, 1 H), 3.29 − 3.36 (m, 2 H), 3.12 − 3.16 (m, 1 H), 2.55 − 2.82 (m, 2 H), 2.42 − 2.53 (m, 1 H), 2.30 − 2.41 (m, 2 H), 2.12 − 2.28 (m, 1 H), 2.02 − 2.12 (m, 1 H). |
| 5 | 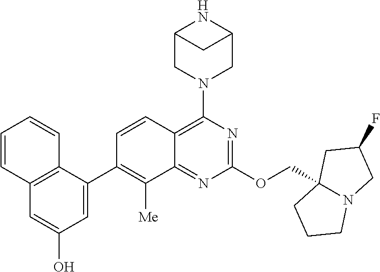 [SMILES: Cc1c(-c2cc(O)cc3ccccc23)ccc2c(N3CC4CC(C3)N4)nc(OC[C@@]34CCCN3C[C@H](F)C4)nc12] | 4-(4-((1R,5S)- 3,6- diazabicyclo[3.1. 1]heptan-3-yl)-2- (((2R,7aS)-2- fluorotetrahydro- 1H-pyrrolizin- 7a(5H)- yl)methoxy)-8- methylquinazolin- 7-yl)naphthalen- 2-ol | 540.2 (M + H)⁺ | ¹H NMR (400 MHz, METHANOL- d4) δ ppm 8.37 − 8.48 (m, 1 H), 7.72 − 7.85 (m, 1 H), 7.37 − 7.47 (m, 2 H), 7.22 − 7.29 (m, 1 H), 7.13 − 7.21 (m, 2 H), 6.96 − 7.03 (m, 1 H), 5.51 − 5.75 (m, 1 H), 4.89 − 4.98 (m, 2 H), 4.81 − 4.84 (m, 2 H), 4.59 − 4.76 (m, 4 H), 3.84 − 4.17 (m, 3 H), 3.44 − 3.59 (m, 1 H), 3.09 − 3.19 (m, 1 H), 2.59 − 2.88 (m, 2 H), 2.34 − 2.55 (m, 3 H), 2.20 − 2.33 (m, 4 H), 1.99 − 2.10 (m, 1 H). |
| 6 | 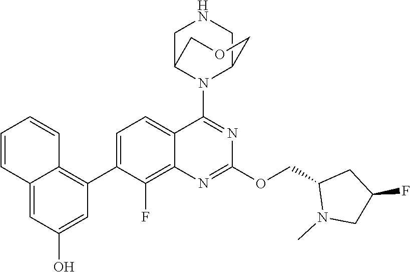 [SMILES: CN1C[C@H](F)C[C@H]1COc1nc(N2C3CNCC2COC3)c2ccc(-c3cc(O)cc4ccccc34)c(F)c2n1] | 4-(4-((1R,5S)-3- oxa-7,9- diazabicyclo[3.3. 1]nonan-9-yl)-8- fluoro-2- (((2S,4R)-4- fluoro-1- methylpyrrolidin- 2- yl)methoxy) quinazolin-7- yl)naphthalen-2- ol | 548.9 (M + H)⁺ | ¹H NMR (400 MHz, METHANOL- d4) δ ppm 7.86 − 7.93 (m, 1 H), 7.75 − 7.81 (m, 1 H), 7.41 − 7.52 (m, 3 H), 7.27 − 7.31 (m, 1 H), 7.20 − 7.26 (m, 1 H), 7.10 − 7.15 (m, 1 H), 5.37 − 5.60 (m, 1 H), 4.99 − 5.11 (m, 1 H), 4.89 − 4.97 (m, 1 H), 4.81 − 4.84 (m, 1 H), 4.78 (br dd, J = 13.4, 5.4 Hz, 1 H), 4.22 − 4.38 (m, 5 H), 3.99 − 4.19 (m, 1 H), 3.58 − 3.88 (m, 5 H), 3.16 − 3.27 (m, 3 H), 2.62 − 2.79 (m, 1 H), 2.32 − 2.56 (m, 1 H). |
| 7 | 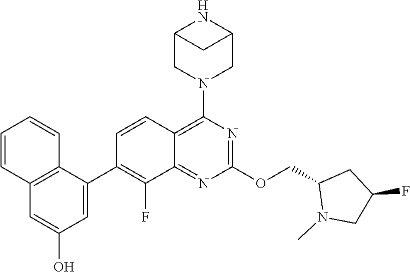 [SMILES: CN1C[C@H](F)C[C@H]1COc1nc(N2CC3CC(C2)N3)c2ccc(-c3cc(O)cc4ccccc34)c(F)c2n1] | 4-(4-((1R,5S)- 3,6- diazabicyclo[3.1. 1]heptan-3-yl)-8- fluoro-2- (((2S,4R)-4- fluoro-1- methylpyrrolidin- 2- yl)methoxy)quina zolin-7- yl)naphthalen-2- ol | 518.8 (M + H)⁺ | ¹H NMR (400 MHz, METHANOL- d4) δ ppm 8.38 − 8.45 (m, 1 H), 7.75 − 7.81 (m, 1 H), 7.42 − 7.52 (m, 3 H), 7.22 − 7.31 (m, 2 H), 7.12 − 7.17 (m, 1 H), 5.37 − 5.61 (m, 1 H), 5.00 − 5.12 (m, 1 H), 4.75 − 4.81 (m, 1 H), 4.60 − 4.72 (m, 4 H), 4.24 − 4.35 (m, 1 H), 3.97 − 4.19 (m, 1 H), 3.57 − 3.75 (m, 1 H), 3.10 − 3.26 (m, 4 H), 2.63 − 2.79 (m, 1 H), 2.28 − 2.54 (m, 1 H), 2.01 − 2.11 (m, 1 H). |
| 8 | 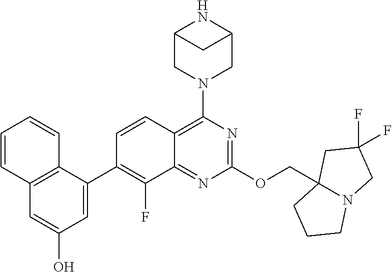 [SMILES: Oc1cc(-c2ccc3c(N4CC5CC(C4)N5)nc(OCC45CCCN4CC(F)(F)C5)nc3c2F)c2ccccc2c1] | 4-(4-((1R,5S)- 3,6- diazabicyclo[3.1. 1]heptan-3-yl)-2- ((2,2- difluorotetrahydro- 1H-pyrrolizin- 7a(5H)- yl)methoxy)-8- fluoroquinazolin- 7-yl)naphthalen- 2-ol | 562.2 (M + H)⁺ | ¹H NMR (400 MHz, METHANOL- d4) δ ppm 8.42 (dd, J = 9.0, 0.8 Hz, 1 H), 7.73 − 7.81 (m, 1 H), 7.40 − 7.52 (m, 3 H), 7.21 − 7.32 (m, 2 H), 7.11 − 7.18 (m, 1 H), 4.77 − 4.82 (m, 2H), 4.76 − 4.82 (m, 2 H), 4.60 − 4.72 (m, 4 H), 4.21 − 4.37 (m, 1 H), 3.83 − 4.01 (m, 2 H), 3.41 − 3.53 (m, 1 H), 3.10 − 3.21 (m, 1 H), 2.79 − 3.07 (m, 2 H), 2.44 − 2.55 (m, 1 H), 2.18 − 2.43 (m, 3 H), 2.01 − 2.11 (m, 1 H). |
| 9 | 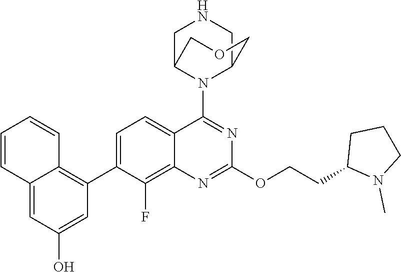 [SMILES: CN1CCC[C@H]1CCOc1nc(N2C3CNCC2COC3)c2ccc(-c3cc(O)cc4ccccc34)c(F)c2n1] | 4-(4-((1R,5S)-3- oxa-7,9- diazabicyclo[3.3. 1]nonan-9-yl)-8- fluoro-2-(2-((S)- 1- methylpyrrolidin- 2- yl)ethoxy)quinaz olin-7- yl)naphthalen-2- ol | 543.8 (M + H)⁺ | ¹H NMR (400 MHz, METHANOL- d4) δ ppm 7.83 − 7.89 (m, 1 H), 7.75 − 7.81 (m, 1 H), 7.41 − 7.48 (m, 3 H), 7.26 − 7.30 (m, 1 H), 7.20 − 7.26 (m, 1 H), 7.10 − 7.13 (m, 1 H), 4.80 − 4.84 (m, 2 H), 4.59 − 4.75 (m, 2 H), 4.30 (br s, 4 H), 3.78 (br s, 5 H), 3.54 − 3.67 (m, 1 H), 3.16 − 3.27 (m, 1 H), 3.02 (s, 3 H), 2.52 (br s, 2 H), 2.04 − 2.26 (m, 3 H), 1.88 − 2.02 (m, 1 H). |
| 10 | 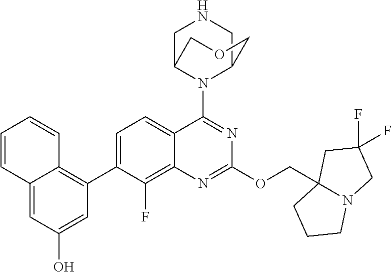 [SMILES: Oc1cc(-c2ccc3c(N4C5CNCC4COC5)nc(OCC45CCCN4CC(F)(F)C5)nc3c2F)c2ccccc2c1] | 4-(4-((1R,5S)-3- oxa-7,9- diazabicyclo[3.3. 1]nonan-9-yl)-2- ((2,2- difluorotetrahydro- 1H-pyrrolizin- 7a(5H)- yl)methoxy)-8- fluoroquinazolin- 7-yl)naphthalen- 2-ol | 592.8 (M + H)⁺ | ¹H NMR (400 MHz, METHANOL- d4) δ ppm 7.85 − 7.94 (m, 1 H), 7.75 − 7.81 (m, 1 H), 7.40 − 7.53 (m, 3 H), 7.26 − 7.31 (m, 1 H), 7.19 − 7.26 (m, 1 H), 7.12 − 7.15 (m, 1 H), 4.89 − 4.93 (m, 2 H), 4.76 − 4.81 (m, 2 H), 4.22 − 4.36 (m, 5 H), 3.86 − 4.02 (m, 2 H), 3.72 − 3.86 (m, 4 H), 3.42 − 3.54 (m, 1 H), 2.94 − 3.11 (m, 1 H), 2.78 − 2.93 (m, 1 H), 2.44 − 2.58 (m, 1 H), 2.21 − 2.44 (m, 3 H). |
| 11 | 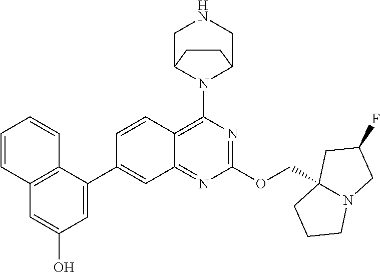 | 4-(4-((1R,5S)- 3,8- diazabicyclo[3.2. 1]octan-8-yl)-2- (((2R,7aS)-2- fluorotetrahydro- 1H-pyrrolizin- 7a(5H)- yl)methoxy) quinazolin-7- yl)naphthalen-2- ol | 540.2 (M + H)⁺ | ¹H NMR (400 MHz, METHANOL- d4) δ ppm 8.29 (d, J = 8.6 Hz, 1 H), 7.76 − 7.80 (m, 2 H), 7.69 (t, J = 9.1 Hz, 2 H), 7.45 (ddd, J = 8.2, 7.0, 1.1 Hz, 1 H), 7.23 − 7.28 (m, 2 H), 7.13 (d, J = 2.5 Hz, 1 H), 5.53 − 5.78 (m, 1 H), 5.39 (br s, 2 H), 4.78 (s, 2 H), 3.89 − 4.05 (m, 3 H), 3.75 (br d, J = 12.5 Hz, 2 H), 3.47 − 3.56 (m, 3 H), 2.59 − 2.88 (m, 2 H), 2.33 − 2.52 (m, 5 H), 2.14 − 2.32 (m, 3 H). |
| 12 | 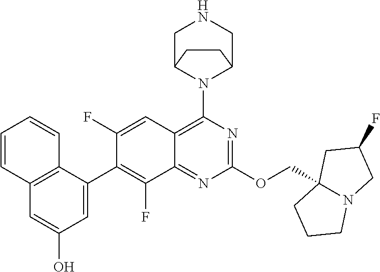 | 4-(4-((1R,5S)- 3,8- diazabicyclo[3.2. 1]octan-8-yl)-6,8- difluoro-2- (((2R,7aS)-2- fluorotetrahydro- 1H-pyrrolizin- 7a(5H)- yl)methoxy) quinazolin-7- yl)naphthalen-2-ol | 576.2 (M + H)⁺ | ¹H NMR (400 MHz, METHANOL- d4) δ ppm 7.75 − 7.88 (m, 2 H), 7.42 − 7.49 (m, 1 H), 7.30 − 7.38 (m, 2 H), 7.21 − 7.28 (m, 1 H), 7.12 − 7.17 (m, 1 H), 5.47 − 5.70 (m, 1 H), 5.17 − 5.27 (m, 2 H), 4.66 − 4.77 (m, 2 H), 3.83 − 4.09 (m, 3 H), 3.66 − 3.76 (m, 2 H), 3.43 − 3.55 (m, 3 H), 2.56 − 2.80 (m, 2 H), 2.31 − 2.52 (m, 5 H), 2.11 − 2.26 (m, 3 H). |
| 13 | 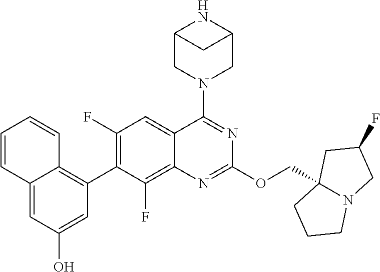 | 4-(4-((1R,5S)- 3,6- diazabicyclo[3.1. 1]heptan-3-yl)- 6,8-difluoro-2- (((2R,7aS)-2- fluorotetrahydro- 1H-pyrrolizin- 7a(5H)- yl)methoxy) quinazolin-7- yl)naphthalen-2-ol | 562.2 (M + H)⁺ | ¹H NMR (400 MHz, METHANOL- d4) δ ppm 8.21 (dd, J = 11.2, 1.6 Hz, 1 H), 7.80 (d, J = 8.2 Hz, 1 H), 7.46 (s, 1 H), 7.32 (br d, J = 2.5 Hz, 2 H), 7.22 − 7.28 (m, 1 H), 7.16 (t, J = 2.1 Hz, 1 H), 5.45 − 5.67 (m, 1 H), 4.57 − 4.83 (m, 8 H), 3.82 − 4.11 (m, 3 H), 3.43 − 3.54 (m, 1 H), 3.10 − 3.23 (m, 1 H), 2.55 − 2.78 (m, 2 H), 2.33 − 2.50 (m, 3 H), 2.10 − 2.24 (m, 1 H), 2.02 − 2.09 (m, 1 H). |
| 14 | 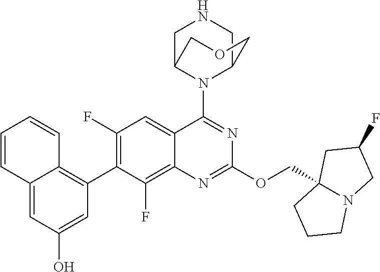 | 4-(4-((1R,5S)-3- oxa-7,9- diazabicyclo[3.3.1] nonan-9-yl)- 6,8-difluoro-2- (((2R,7aS)-2- fluorotetrahydro- 1H-pyrrolizin- 7a(5H)- yl)methoxy) quinazolin-7- yl)naphthalen-2- ol | 592.2 (M + H)⁺ | ¹H NMR (400 MHz, METHANOL- d4) δ ppm 7.79 (d, J = 8.4 Hz, 1 H), 7.61 − 7.69 (m, 1 H), 7.46 (s, 1 H), 7.32 (br d, J = 2.5 Hz, 2 H), 7.20 − 7.28 (m, 1 H), 7.14 (d, J = 2.1 Hz, 1 H), 5.44 − 5.71 (m, 1 H), 4.83 (br s, 2 H), 4.72 (s, 2 H), 4.29 (s, 4 H), 3.87 − 4.11 (m, 3 H), 3.78 (s, 4 H), 3.44 − 3.54 (m, 1 H), 2.55 − 2.81 (m, 2 H), 2.29 − 2.52 (m, 3 H), 2.14 − 2.28 (m, 1 H). |
| 15 | 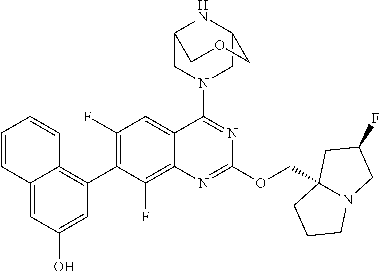 | 4-(4-((1R,5S)-3- oxa-7,9- diazabicyclo[3.3. 1]nonan-7-yl)- 6,8-difluoro-2- (((2R,7aS)-2- fluorotetrahydro- 1H-pyrrolizin- 7a(5H)- yl)methoxy) quinazolin-7- yl)naphthalen-2- ol | 592.2 (M + H)⁺ | ¹H NMR (500 MHz, DMSO-d6) δ ppm 8.10 − 8.18 (m, 1 H) 7.81 (d, J = 8.31 Hz, 1 H) 7.73 − 7.79 (m, 1 H) 7.45 (s, 1 H) 7.32 − 7.38 (m, 1 H) 7.30 (d, J = 2.20 Hz, 1 H) 7.22 − 7.27 (m, 1 H) 7.15 (dd, J = 2.26, 1.16 Hz, 1 H) 5.17 − 5.43 (m, 1 H) 4.67 (br t, J = 13.27 Hz, 2 H) 4.06 − 4.13 (m, 1 H) 3.96 − 4.06 (m, 1 H) 3.81 − 3.89 (m, 2 H) 3.69 − 3.80 (m, 4 H) 2.98 − 3.21 (m, 3 H) 2.92 (br s, 2 H) 2.73 − 2.85 (m, 1 H) 2.05 (br s, 3 H) 1.77 (br s, 3 H). |
| 16 | 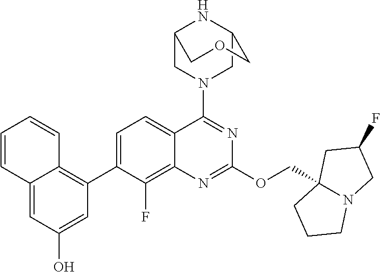 | 4-(4-((1R,5S)-3- oxa-7,9- diazabicyclo[3.3. 1]nonan-7-yl)-8- fluoro-2- (((2R,7aS)-2- fluorotetrahydro- 1H-pyrrolizin- 7a(5H)- yl)methoxy) quinazolin-7- yl)naphthalen-2- ol | 574.2 (M + H)⁺ | ¹H NMR (500 MHz, DMSO-d6) δ ppm 7.92 (d, J = 8.56 Hz, 1 H) 7.79 (d, J = 8.19 Hz, 1 H) 7.39 − 7.48 (m, 2 H) 7.17 − 7.32 (m, 4 H) 7.09 (d, J = 2.45 Hz, 1 H) 5.13 − 5.40 (m, 1 H) 4.61 − 4.76 (m, 2 H) 3.95 − 4.17 (m, 3 H) 3.87 (br s, 2 H) 3.66 − 3.77 (m, 3 H) 2.97 − 3.20 (m, 4 H) 2.83 (br d, J = 6.36 Hz, 1 H) 1.98 − 2.24 (m, 3 H) 1.71 − 1.92 (m, 3 H). |
| 17 | 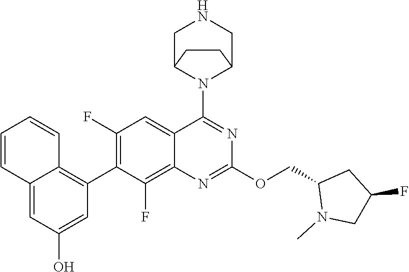 | 4-(4-((1R,5S)- 3,8- diazabicyclo[3.2. 1]octan-8-yl)-6,8- difluoro-2- (((2S,4R)-4- fluoro-1- methylpyrrolidin- 2- yl)methoxy) quinazolin-7- yl)naphthalen-2- ol | 550.2 (M + H)⁺ | ¹H NMR (400 MHz, METHANOL- d4) δ ppm 7.75 − 7.87 (m, 2 H), 7.42 − 7.51 (m, 1 H), 7.29 − 7.40 (m, 2 H), 7.21 − 7.28 (m, 1 H), 7.12 − 7.18 (m, 1 H), 5.39 − 5.58 (m, 1 H), 5.16 − 5.26 (m, 2 H), 4.97 − 5.06 (m, 1 H), 4.71 − 4.79 (m, 1 H), 4.21 − 4.39 (m, 1 H), 3.97 − 4.21 (m, 1 H), 3.57 − 3.78 (m, 3 H), 3.47 (br d, J = 11.3 Hz, 2 H), 3.21 (s, 3 H), 2.67 (s, 1 H), 2.27 − 2.54 (m, 3 H), 2.17 (d, J = 8.4 Hz, 2 H). |
| 18 | 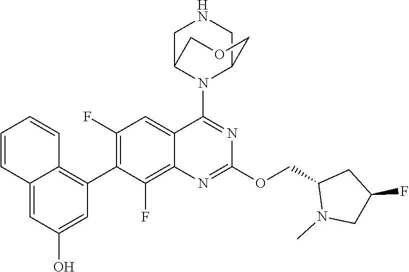 [SMILES: CN1C[C@H](F)C[C@H]1COc1nc(N2C3CNCC2COC3)c2cc(F)c(-c3cc(O)cc4ccccc34)c(F)c2n1] | 4-(4-((1R,5S)-3- oxa-7,9- diazabicyclo[3.3. 1]nonan-9-yl)- 6,8-difluoro-2- (((2S,4R)-4- fluoro-1- methylpyrrolidin- 2- yl)methoxy) quinazolin-7- yl)naphthalen-2-ol | 566.2 (M + H)⁺ | ¹H NMR (400 MHz, METHANOL- d4) δ ppm 7.76 − 7.84 (m, 1 H), 7.62 − 7.70 (m, 1 H), 7.42 − 7.49 (m, 1 H), 7.30 − 7.38 (m, 2 H), 7.21 − 7.29 (m, 1 H), 7.11 − 7.17 (m, 1 H), 5.36 − 5.61 (m, 1 H), 4.97 − 5.09 (m, 1 H), 4.71 − 4.83 (m, 3 H), 4.29 (br s, 5 H), 3.97 − 4.21 (m, 1 H), 3.59 − 3.88 (m, 5 H), 3.21 (s, 3 H), 2.63 − 2.78 (m, 1 H), 2.32 − 2.56 (m, 1 H). |
| 19 | 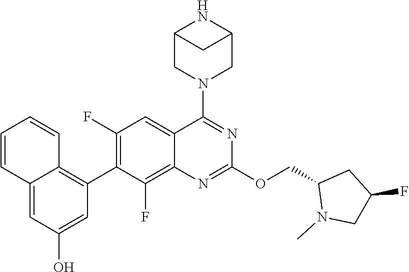 [SMILES: CN1C[C@H](F)C[C@H]1COc1nc(N2CC3CC(C2)N3)c2cc(F)c(-c3cc(O)cc4ccccc34)c(F)c2n1] | 4-(4-((1R,5S)- 3,6- diazabicyclo[3.1. 1]heptan-3-yl)- 6,8-difluoro-2- (((2S,4R)-4- fluoro-1- methylpyrrolidin- 2- yl)methoxy) quinazolin-7- yl)naphthalen-2- ol | 536.2 (M + H)⁺ | ¹H NMR (400 MHz, METHANOL- d4) δ ppm 8.17 − 8.25 (m, 1 H), 7.75 − 7.83 (m, 1 H), 7.43 − 7.51 (m, 1 H), 7.30 − 7.39 (m, 2 H), 7.22 − 7.29 (m, 1 H), 7.13 − 7.19 (m, 1 H), 5.34 − 5.61 (m, 1 H), 4.97 − 5.10 (m, 1 H), 4.81 (br s, 3 H), 4.65 (br d, J = 6.1 Hz, 4 H), 4.21 − 4.36 (m, 1 H), 3.98 − 4.18 (m, 1 H), 3.59 − 3.76 (m, 1 H), 3.20 (d, J = 1.0 Hz, 4 H), 2.62 − 2.77 (m, 1 H), 2.28 − 2.53 (m, 1 H), 2.06 (d, J = 11.1 Hz, 1 H). |
| 20 | 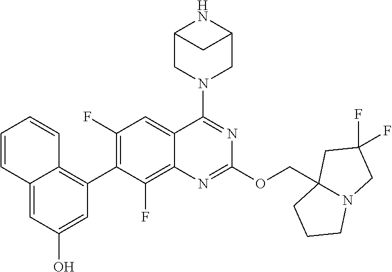 [SMILES: Oc1cc(-c2c(F)cc3c(N4CC5CC(C4)N5)nc(OCC45CCCN4CC(F)(F)C5)nc3c2F)c2ccccc2c1] | 4-(4-((1R,5S)- 3,6- diazabicyclo[3.1. 1]heptan-3-yl)-2- ((2,2- difluorotetrahydro- 1H-pyrrolizin- 7a(5H)- yl)methoxy)-6,8- difluoroquinazolin- 7-yl)naphthalen-2- ol | 579.8 (M + H)⁺ | ¹H NMR (400 MHz, METHANOL- d4) δ ppm 8.18 − 8.26 (m, 1 H), 7.76 − 7.84 (m, 1 H), 7.42 − 7.50 (m, 1 H), 7.31 − 7.40 (m, 2 H), 7.21 − 7.28 (m, 1 H), 7.16 (s, 1 H), 4.77 (s, 4 H), 4.65 (br s, 4 H), 4.17 − 4.36 (m, 1 H), 3.82 − 4.02 (m, 2 H), 3.39 − 3.54 (m, 1 H), 3.10 − 3.21 (m, 1 H), 2.79 − 3.07 (m, 2 H), 2.18 − 2.54 (m, 4 H), 2.07 (d, J = 10.6 Hz, 1 H). |
| 21 | 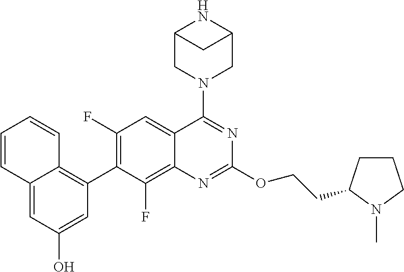 [SMILES: CN1CCC[C@H]1CCOc1nc(N2CC3CC(C2)N3)c2cc(F)c(-c3cc(O)cc4ccccc34)c(F)c2n1] | 4-(4-((1R,5S)- 3,6- diazabicyclo[3.1. 1]heptan-3-yl)- 6,8-difluoro-2-(2- ((S)-1- methylpyrrolidin- 2- yl)ethoxy) quinazolin-7- yl)naphthalen-2- ol | 531.8 (M + H)⁺ | ¹H NMR (400 MHz, METHANOL- d4) δ ppm 8.13 − 8.23 (m, 1 H), 7.77 − 7.83 (m, 1 H), 7.40 − 7.49 (m, 1 H), 7.32 (d, J = 2.5 Hz, 2 H), 7.21 − 7.28 (m, 1 H), 7.15 (d, J = 2.5 Hz, 1 H), 4.80 − 4.84 (m, 2 H), 4.59 − 4.74 (m, 6 H), 3.67 − 3.82 (m, 1 H), 3.56 − 3.66 (m, 1 H), 3.10 − 3.28 (m, 2 H), 3.01 (s, 3 H), 2.39 − 2.59 (m, 2 H), 2.12 − 2.23 (m, 2 H), 2.01 − 2.12 (m, 2 H), 1.88 − 2.01 (m, 1 H). |
| 22 | 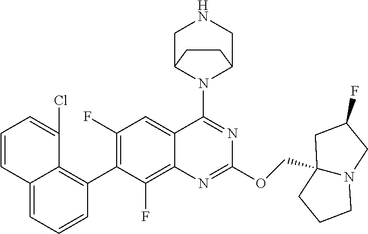 [SMILES: Fc1cc2c(N3C4CCC3CNC4)nc(OC[C@@]34CCCN3C[C@H](F)C4)nc2c(F)c1-c1cccc2cccc(Cl)c12] | 4-((1R,5S)-3,8- diazabicyclo[3.2. 1]octan-8-yl)-7-(8- chloronaphthalen- 1-yl)-6,8- difluoro-2- (((2R,7aS)-2- fluorotetrahydro- 1H-pyrrolizin- 7a(5H)- yl)methoxy) quinazoline | 594.2 (M + H)⁺ | ¹H NMR (400 MHz, METHANOL- d4) δ ppm 8.16 (s, 1 H), 8.01 − 8.08 (m, 1 H), 7.67 − 7.78 (m, 2 H), 7.63 (dd, J = 7.5, 1.0 Hz, 1 H), 7.49 − 7.57 (m, 2 H), 5.42 − 5.68 (m, 1 H), 5.10 − 5.33 (m, 2 H), 4.70 (d, J = 3.1 Hz, 2 H), 3.81 − 4.10 (m, 3 H), 3.61 − 3.81 (m, 2 H), 3.47 (br d, J = 12.6 Hz, 3 H), 2.55 − 2.82 (m, 2 H), 2.37 (br s, 5 H), 2.16 (br d, J = 8.9 Hz, 3 H). |
| 23 | 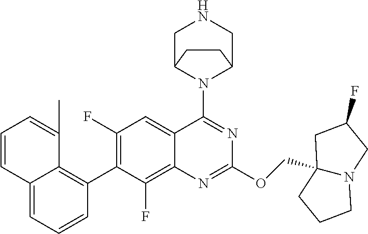 [SMILES: Cc1cccc2cccc(-c3c(F)cc4c(N5C6CCC5CNC6)nc(OC[C@@]56CCCN5C[C@H](F)C6)nc4c3F)c12] | 4-((1R,5S)-3,8- diazabicyclo[3.2. 1]octan-8-yl)-6,8- difluoro-2- (((2R,7aS)-2- fluorotetrahydro- 1H-pyrrolizin- 7a(5H)- yl)methoxy)-7-(8- methylnaphthalen- 1-yl)quinazoline | 574.2 (M + H)⁺ | ¹H NMR (400 MHz, METHANOL- d4) δ ppm 8.03 − 8.10 (m, 1 H), 7.87 − 7.93 (m, 1 H), 7.75 − 7.81 (m, 1 H), 7.55 − 7.63 (m, 1 H), 7.42 − 7.49 (m, 1 H), 7.37 − 7.42 (m, 1 H), 7.31 − 7.36 (m, 1 H), 5.48 − 5.68 (m, 1 H), 5.15 − 5.27 (m, 2 H), 4.70 (s, 2H), 3.83 − 4.07 (m, 3 H), 3.64 − 3.79 (m, 2 H), 3.43 − 3.54 (m, 3 H), 2.55 − 2.81 (m, 2 H), 2.36 (br dd, J = 11.7, 5.2 Hz, 5 H), 2.08 − 2.26 (m, 6 H). |
| 24 | 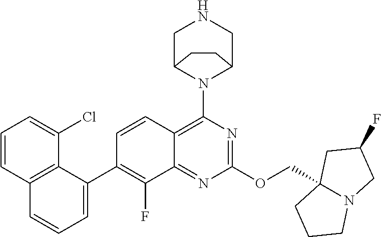 [SMILES: Fc1c(-c2cccc3cccc(Cl)c23)ccc2c(N3C4CCC3CNC4)nc(OC[C@@]34CCCN3C[C@H](F)C4)nc12] | 4-((1R,5S)-3,8- diazabicyclo[3.2. 1]octan-8-yl)-7-(8- chloronaphthalen- 1-yl)-8-fluoro-2- (((2R,7aS)-2- fluorotetrahydro- 1H-pyrrolizin- 7a(5H)- yl)methoxy) quinazoline | 576.3 (M + H)⁺ | ¹H NMR (400 MHz, METHANOL- d4) δ ppm 8.10 − 8.16 (m, 1 H), 7.97 − 8.07 (m, 2 H), 7.65 − 7.72 (m, 1 H), 7.58 − 7.63 (m, 1 H), 7.45 − 7.56 (m, 3 H), 5.48 − 5.68 (m, 1 H), 5.23 − 5.36 (m, 2 H), 4.65 − 4.79 (m, 2 H), 3.82 − 4.09 (m, 3 H), 3.65 − 3.78 (m, 2 H), 3.42 − 3.56 (m, 3 H), 2.53 − 2.80 (m, 2 H), 2.28 − 2.52 (m, 5 H), 2.08 − 2.25 (m, 3 H). |
| 25 | 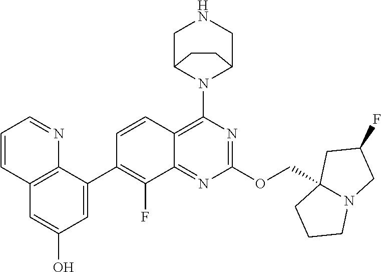 [SMILES: Oc1cc(-c2ccc3c(N4C5CCC4CNC5)nc(OC[C@@]45CCCN4C[C@H](F)C5)nc3c2F)c2ncccc2c1] | 8-(4-((1R,5S)-3,8- diazabicyclo[3.2. 1]octan-8-yl)-8- fluoro-2- (((2R,7aS)-2- fluorotetrahydro- 1H-pyrrolizin- 7a(5H)- yl)methoxy) quinazolin-7- yl)quinolin-6-ol | 559.9 (M + H)⁺ | ¹H NMR (400 MHz, METHANOL- d4) δ ppm 8.73 (dd, J = 4.8, 1.7 Hz, 1 H), 8.60 − 8.69 (m, 1 H), 8.02 − 8.12 (m, 1 H), 7.71 − 7.79 (m, 1 H), 7.54 − 7.62 (m, 2 H), 7.45 − 7.52 (m, 1 H), 5.47 − 5.70 (m, 1 H), 5.25 − 5.36 (m, 2 H), 4.71 − 4.79 (m, 2 H), 3.82 − 4.14 (m, 3 H), 3.66 − 3.77 (m, 2 H), 3.42 − 3.55 (m, 3 H), 2.57 − 2.79 (m, 2 H), 2.29 − 2.53 (m, 5 H), 2.11 − 2.26 (m, 3 H). |
| 26 | 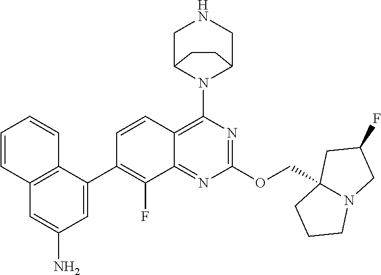 [SMILES: Nc1cc(-c2ccc3c(N4C5CCC4CNC5)nc(OC[C@@]45CCCN4C[C@H](F)C5)nc3c2F)c2ccccc2c1] | 4-(4-((1R,5S)-3,8- diazabicyclo[3.2. 1]octan-8-yl)-8- fluoro-2- (((2R,7aS)-2- fluorotetrahydro- 1H-pyrrolizin- 7a(5H)- yl)methoxy) quinazolin-7- yl)naphthalen-2- amine | 557.3 (M + H)⁺ | ¹H NMR (400 MHz, METHANOL- d4) δ ppm 8.05 − 8.11 (m, 1 H), 7.91 − 7.97 (m, 1 H), 7.70 − 7.75 (m, 1 H), 7.53 − 7.61 (m, 2 H), 7.47 − 7.53 (m, 1 H), 7.40 − 7.45 (m, 1 H), 7.36 − 7.39 (m, 1 H), 5.49 − 5.71 (m, 1 H), 5.23 − 5.34 (m, 2 H), 4.69 − 4.77 (m, 2 H), 3.82 − 4.12 (m, 3 H), 3.66 − 3.78 (m, 2 H), 3.43 − 3.55 (m, 3 H), 2.55 − 2.83 (m, 2 H), 2.30 − 2.52 (m, 5 H), 2.12 − 2.29 (m, 3 H). |
| 27 | 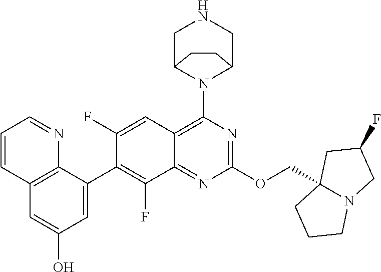 [SMILES: Oc1cc(-c2c(F)cc3c(N4C5CCC4CNC5)nc(OC[C@@]45CCCN4C[C@H](F)C5)nc3c2F)c2ncccc2c1] | 8-(4-((1R,5S)-3,8- diazabicyclo[3.2. 1]octan-8-yl)-6,8- difluoro-2- (((2R,7aS)-2- fluorotetrahydro- 1H-pyrrolizin- 7a(5H)- yl)methoxy) quinazolin-7- yl)quinolin-6-ol | 577.9 (M + H)⁺ | ¹H NMR (400 MHz, METHANOL- d4) δ ppm 8.63 − 8.71 (m, 1 H), 8.47 − 8.55 (m, 1 H), 7.78 − 7.86 (m, 1 H), 7.63 − 7.69 (m, 1 H), 7.52 − 7.58 (m, 1 H), 7.42 − 7.48 (m, 1 H), 5.47 − 5.72 (m, 1 H), 5.16 − 5.32 (m, 2 H), 4.64 − 4.79 (m, 2 H), 3.82 − 4.12 (m, 3 H), 3.64 − 3.79 (m, 2 H), 3.42 − 3.55 (m, 3 H), 2.54 − 2.84 (m, 2 H), 2.28 − 2.52 (m, 5 H), 2.10 − 2.26 (m, 3 H). |
| 28 | 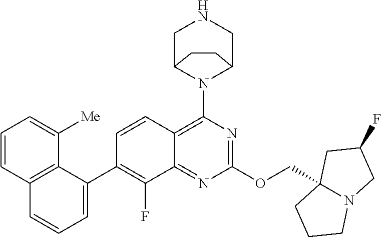 [SMILES: Cc1cccc2cccc(-c3ccc4c(N5C6CCC5CNC6)nc(OC[C@@]56CCCN5C[C@H](F)C6)nc4c3F)c12] | 4-((1R,5S)-3,8- diazabicyclo[3.2. 1]octan-8-yl)-8- fluoro-2- (((2R,7aS)-2- fluorotetrahydro- 1H-pyrrolizin- 7a(5H)- yl)methoxy)-7-(8- methylnaphthalen- 1-yl)quinazoline | 556.3 (M + H)⁺ | ¹H NMR (400 MHz, METHANOL- d4) δ ppm 7.98 − 8.05 (m, 2 H), 7.85 − 7.90 (m, 1 H), 7.53 − 7.60 (m, 1 H), 7.40 − 7.50 (m, 2 H), 7.29 − 7.39 (m, 2 H), 5.48 − 5.69 (m, 1 H), 5.21 − 5.36 (m, 2 H), 4.66 − 4.79 (m, 2 H), 3.82 − 4.12 (m, 3 H), 3.65 − 3.75 (m, 2 H), 3.41 − 3.53 (m, 3 H), 2.54 − 2.81 (m, 2 H), 2.30 − 2.52 (m, 5 H), 2.13 − 2.25 (m, 3 H), 2.03 − 2.12 (m, 3 H). |
| 29 | 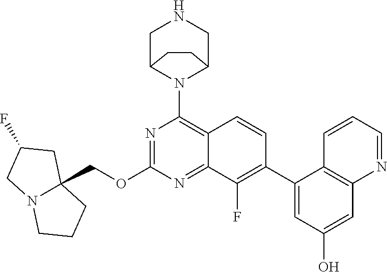 [SMILES: Oc1cc(-c2ccc3c(N4C5CCC4CNC5)nc(OC[C@@]45CCCN4C[C@H](F)C5)nc3c2F)c2cccnc2c1] | 5-(4-((1R,5S)-3,8- diazabicyclo[3.2. 1]octan-8-yl)-8- fluoro-2- (((2R,7aS)-2- fluorotetrahydro- 1H-pyrrolizin- 7a(5H)- yl)methoxy) quinazolin-7- yl)quinolin-7-ol | 559.2 (M + H)⁺ | ¹H NMR (400 MHz, METHANOL- d4) δ ppm 9.03 (dd, J = 5.4, 1.5 Hz, 1 H), 8.56 − 8.67 (m, 1 H), 8.07 − 8.16 (m, 1 H), 7.70 − 7.82 (m, 1 H), 7.55 − 7.61 (m, 2 H), 7.48 − 7.55 (m, 1 H), 5.50 − 5.73 (m, 1 H), 5.20 − 5.34 (m, 2 H), 4.68 − 4.78 (m, 2 H), 3.84 − 4.17 (m, 3 H), 3.65 − 3.81 (m, 2 H), 3.42 − 3.56 (m, 3 H), 2.56 − 2.84 (m, 2 H), 2.29 − 2.53 (m, 5 H), 2.10 − 2.28 (m, 3 H). |
| 30 | 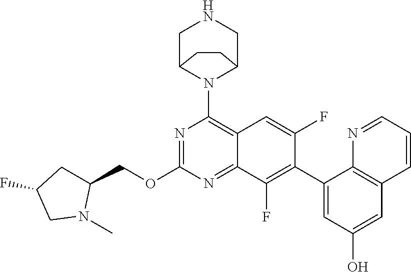 [SMILES: CN1C[C@H](F)C[C@H]1COc1nc(N2C3CCC2CNC3)c2cc(F)c(-c3cc(O)cc4cccnc34)c(F)c2n1] | 8-(4-((1R,5S)- 3,8- diazabicyclo[3.2. 1]octan-8-yl)-6,8- difluoro-2- (((2S,4R)-4- fluoro-1- methylpyrrolidin- 2- yl)methoxy) quinazolin-7- yl)quinolin-6-ol | 551.8 (M + H)⁺ | ¹H NMR (400 MHz, METHANOL- d4) δ ppm 8.62 − 8.71 (m, 1 H), 8.45 − 8.54 (m, 1 H), 7.77 − 7.85 (m, 1 H), 7.61 − 7.70 (m, 1 H), 7.52 − 7.58 (m, 1 H), 7.43 − 7.50 (m, 1 H), 5.38 − 5.65 (m, 1 H), 5.15 − 5.27 (m, 2 H), 4.94 − 5.11 (m, 1 H), 4.68 − 4.80 (m, 1 H), 3.98 − 4.35 (m, 2 H), 3.57 − 3.79 (m, 3 H), 3.40 − 3.53 (m, 2 H), 3.14 − 3.27 (m, 3 H), 2.61 − 2.79 (m, 1 H), 2.28 − 2.55 (m, 3 H), 2.08 − 2.24 (m, 2 H). |
| 31 | 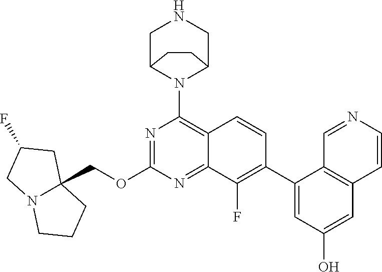 [SMILES: Oc1cc(-c2ccc3c(N4C5CCC4CNC5)nc(OC[C@@]45CCCN4C[C@H](F)C5)nc3c2F)c2cnccc2c1] | 8-(4-((1R,5S)- 3,8- diazabicyclo[3.2. 1]octan-8-yl)-8- fluoro-2- (((2R,7aS)-2- fluorotetrahydro- 1H-pyrrolizin- 7a(5H)- yl)methoxy) quinazolin-7- yl)isoquinolin-6- ol | 559.3 (M + H)⁺ | ¹H NMR (400 MHz, METHANOL- d4) δ ppm 9.04 − 9.16 (m, 1 H), 8.40 − 8.45 (m, 1 H), 8.25 − 8.30 (m, 1 H), 8.09 − 8.16 (m, 1 H), 7.60 − 7.64 (m, 1 H), 7.55 − 7.60 (m, 2 H), 5.48 − 5.74 (m, 1 H), 5.16 − 5.38 (m, 2 H), 4.62 − 4.79 (m, 2 H), 3.83 − 4.18 (m, 3 H), 3.64 − 3.80 (m, 2 H), 3.41 − 3.58 (m, 3 H), 2.56 − 2.87 (m, 2 H), 2.43 − 2.54 (m, 1 H), 2.29 − 2.42 (m, 4 H), 2.09 − 2.26 (m, 3 H). |
| 32 | 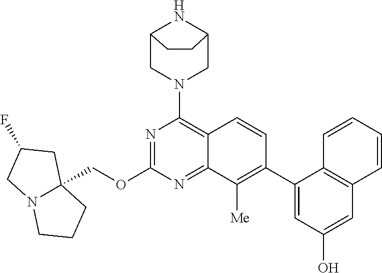 [SMILES: Cc1c(-c2cc(O)cc3ccccc23)ccc2c(N3CC4CCC(C3)N4)nc(OC[C@]34CCCN3C[C@H](F)C4)nc12] | 4-(4-((1R,5S)- 3,8- diazabicyclo[3.2. 1]octan-3-yl)-2- (((2R,7aS)-2- fluorotetrahydro- 1H-pyrrolizin- 7a(5H)- yl)methoxy)-8- methylquinazolin- 7-yl)naphthalen- 2-ol | 554.2 (M + H)⁺ | ¹H NMR (400 MHz, METHANOL- d4) δ ppm 7.95 − 8.02 (m, 1 H), 7.73 − 7.80 (m, 1 H), 7.39 − 7.45 (m, 1 H), 7.35 − 7.39 (m, 1 H), 7.23 − 7.25 (m, 1 H), 7.14 − 7.23 (m, 2 H), 6.96 − 7.01 (m, 1 H), 5.43 − 5.66 (m, 1 H), 4.87 − 4.92 (m, 1 H), 4.79 − 4.83 (m, 1 H), 4.65 − 4.77 (m, 2 H), 4.22 − 4.31 (m, 2 H), 4.04 − 4.17 (m, 1 H), 3.85 − 3.96 (m, 2 H), 3.67 − 3.79 (m, 1 H), 3.46 − 3.63 (m, 2 H), 2.69 − 2.82 (m, 1 H), 2.40 − 2.62 (m, 2 H), 2.28 − 2.38 (m, 5 H), 2.11 − 2.27 (m, 5 H). |
| 33 | 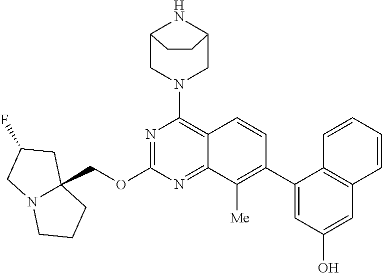 [SMILES: Cc1c(-c2cc(O)cc3ccccc23)ccc2c(N3CC4CCC(C3)N4)nc(OC[C@@]34CCCN3C[C@H](F)C4)nc12] | 4-(4-((1R,5S)- 3,8- diazabicyclo[3.2. 1]octan-3-yl)-2- (((2R,7aS)-2- fluorotetrahydro- 1H-pyrrolizin- 7a(5H)- yl)methoxy)-8- methylquinazolin- 7-yl)naphthalen- 2-ol | 554.4 (M + H)⁺ | ¹H NMR (400 MHz, METHANOL- d4) δ ppm 7.94 − 8.01 (m, 1 H), 7.74 − 7.80 (m, 1 H), 7.39 − 7.45 (m, 1 H), 7.32 − 7.38 (m, 1 H), 7.22 − 7.25 (m, 1 H), 7.14 − 7.22 (m, 2 H), 6.95 − 7.00 (m, 1 H), 5.50 − 5.73 (m, 1 H), 4.72 − 4.78 (m, 2 H), 4.60 − 4.71 (m, 2 H), 4.21 − 4.31 (m, 2 H), 3.90 − 4.13 (m, 3 H), 3.80 − 3.89 (m, 2 H), 3.46 − 3.59 (m, 1 H), 2.56 − 2.89 (m, 2 H), 2.45 − 2.55 (m, 1 H), 2.34 − 2.44 (m, 2 H), 2.30 − 2.34 (m, 3 H), 2.12 − 2.28 (m, 5 H). |
| 34 | 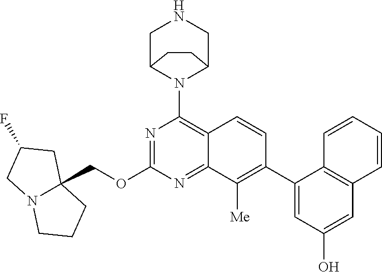 [SMILES: Cc1c(-c2cc(O)cc3ccccc23)ccc2c(N3C4CCC3CNC4)nc(OC[C@@]34CCCN3C[C@H](F)C4)nc12] | 4-(4-((1R,5S)- 3,8- diazabicyclo[3.2. 1]octan-8-yl)-2- (((2R,7aS)-2- fluorotetrahydro- 1H-pyrrolizin- 7a(5H)- yl)methoxy)-8- methylquinazolin- 7-yl)naphthalen- 2-ol | 554.4 (M + H)⁺ | ¹H NMR (400 MHz, METHANOL- d4) δ ppm 8.01 − 8.09 (m, 1 H), 7.73 − 7.82 (m, 1 H), 7.37 − 7.45 (m, 2 H), 7.14 − 7.26 (m, 3 H), 6.96 − 7.01 (m, 1 H), 5.51 − 5.73 (m, 1 H), 5.17 − 5.29 (m, 2 H), 4.71 − 4.79 (m, 2 H), 3.85 − 4.13 (m, 3 H), 3.68 − 3.79 (m, 2 H), 3.44 − 3.57 (m, 3 H), 2.57 − 2.87 (m, 2 H), 2.44 − 2.55 (m, 1 H), 2.21 − 2.44 (m, 8 H), 2.11 − 2.21 (m, 2 H). |
| 35 | 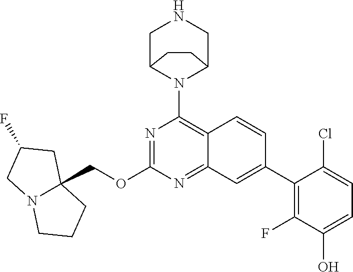 [SMILES: Oc1ccc(Cl)c(-c2ccc3c(N4C5CCC4CNC5)nc(OC[C@@]45CCCN4C[C@H](F)C5)nc3c2)c1F] | 3-(4-((1R,5S)- 3,8- diazabicyclo[3.2. 1]octan-8-yl)-2- (((2R,7aS)-2- fluorotetrahydro- 1H-pyrrolizin- 7a(5H)- yl)methoxy) quinazolin-7-yl)-4- chloro-2- fluorophenol | 542.2 (M + H)⁺ | ¹H NMR (400 MHz, DMSO-d₆) δ ppm 10.71 − 10.88 (m, 1 H), 10.21 − 10.53 (m, 1 H), 9.36 − 9.64 (m, 1 H), 8.72 − 9.05 (m, 1 H), 8.22 (d, J = 8.6 Hz, 1 H), 7.61 (d, J = 1.5 Hz, 1 H), 7.39 (dd, J = 8.6, 0.8 Hz, 1 H), 7.28 (dd, J = 8.8, 1.7 Hz, 1 H), 7.08 (t, J = 9.0 Hz, 1 H), 5.51 − 5.68 (m, 1 H), 5.06 (br s, 2 H), 4.59 (d, J = 1.5 Hz, 2 H), 3.71 − 3.93 (m, 4 H), 3.49 (br d, J = 11.7 Hz, 2 H), 3.36 (br d, J = 12.1 Hz, 3 H), 2.00 − 2.23 (m, 7 H) |
| 36 | 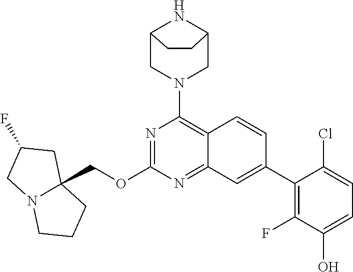 [SMILES: Oc1ccc(Cl)c(-c2ccc3c(N4CC5CCC(C4)N5)nc(OC[C@@]45CCCN4C[C@H](F)C5)nc3c2)c1F] | 3-(4-((1R,5S)- 3,8- diazabicyclo[3.2. 1]octan-3-yl)-2- (((2R,7aS)-2- fluorotetrahydro- 1H-pyrrolizin- 7a(5H)- yl)methoxy) quinazolin-7-yl)- 4-chloro-2- fluorophenol | 542.2 (M + H)⁺ | ¹H NMR (400 MHz, DMSO-d₆) δ ppm 10.65 − 10.87 (m, 1 H), 10.25 − 10.48 (m, 1 H), 9.15 − 9.34 (m, 1 H), 8.95 − 9.12 (m, 1 H), 8.12 (d, J = 8.6 Hz, 1 H), 7.61 (d, J = 1.7 Hz, 1 H), 7.33 − 7.41 (m, 1 H), 7.27 (dd, J = 8.8, 1.7 Hz, 1 H), 7.07 (t, J = 9.0 Hz, 1 H), 5.49 − 5.68 (m, 1 H), 4.59 (s, 2 H), 4.45 (br d, J = 14.6 Hz, 2 H), 4.20 (br s, 3 H), 3.69 − 3.85 (m, 6 H), 3.29 − 3.37 (m, 1 H), 1.97 − 2.19 (m, 6 H). |
| 37 | 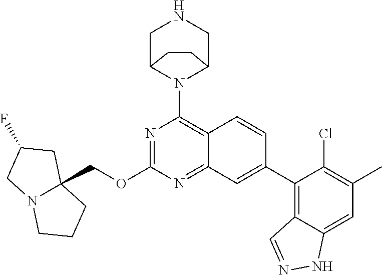 [SMILES: Cc1cc2[nH]ncc2c(-c2ccc3c(N4C5CCC4CNC5)nc(OC[C@@]45CCCN4C[C@H](F)C5)nc3c2)c1Cl] | 4-((1R,5S)-3,8- diazabicyclo[3.2. 1]octan-8-yl)-7- (5-chloro-6- methyl-1H- indazol-4-yl)-2- (((2R,7aS)-2- fluorotetrahydro- 1H-pyrrolizin- 7a(5H)- yl)methoxy) quinazoline | 562.2 (M + H)⁺ | ¹H NMR (400 MHz, DMSO-d₆) δ ppm 10.68 − 10.91 (m, 1 H), 9.30 − 9.64 (m, 1 H), 8.80 − 9.06 (m, 1 H), 8.27 (d, J = 8.6 Hz, 1 H), 7.72 (d, J = 1.9 Hz, 1 H), 7.62 − 7.68 (m, 2 H), 7.53 (dd, J = 8.6, 1.9 Hz, 1 H), 5.51 − 5.67 (m, 1 H), 5.09 (br s, 2 H), 4.61 (d, J = 1.7 Hz, 2 H), 3.75 − 3.95 (m, 3 H), 3.49 − 3.60 (m, 2 H), 3.30 − 3.43 (m, 3 H), 2.54 − 2.57 (m, 3 H), 2.01 − 2.35 (m, 9 H). |
| 38 | 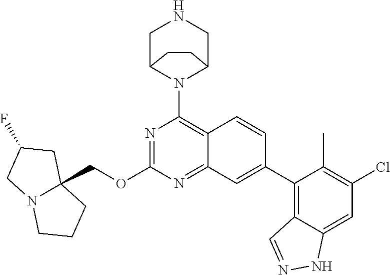 [SMILES: Cc1c(Cl)cc2[nH]ncc2c1-c1ccc2c(N3C4CCC3CNC4)nc(OC[C@@]34CCCN3C[C@H](F)C4)nc2c1] | 4-((1R,5S)-3,8- diazabicyclo[3.2. 1]octan-8-yl)-7- (6-chloro-5- methyl-1H- indazol-4-yl)-2- (((2R,7aS)-2- fluorotetrahydro- 1H-pyrrolizin- 7a(5H)- yl)methoxy) quinazoline | 562.2 (M + H)⁺ | ¹H NMR (400 MHz, DMSO-d₆) δ ppm 10.64 − 10.95 (m, 1 H), 9.36 − 9.57 (m, 1 H), 8.76 − 9.06 (m, 1 H), 8.26 (d, J = 8.6 Hz, 1 H), 7.78 (s, 1 H), 7.63 (dd, J = 17.5, 1.4 Hz, 2 H), 7.47 (dd, J = 8.5, 1.8 Hz, 1 H), 5.50 − 5.70 (m, 1 H), 5.09 (br d, J = 0.6 Hz, 2 H), 4.60 (s, 2 H), 3.73 − 3.93 (m, 3 H), 3.47 − 3.58 (m, 2 H), 3.28 − 3.43 (m, 3 H), 1.98 − 2.32 (m, 12 H). |
| 39 | 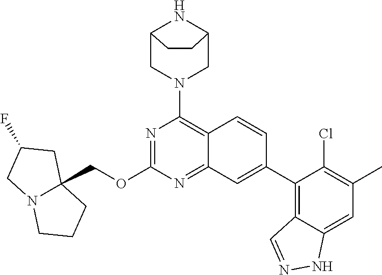 [SMILES: Cc1cc2[nH]ncc2c(-c2ccc3c(N4CC5CCC(C4)N5)nc(OC[C@@]45CCCN4C[C@H](F)C5)nc3c2)c1Cl] | 4-((1R,5S)-3,8- diazabicyclo[3.2. 1]octan-3-yl)-7- (5-chloro-6- methyl-1H- indazol-4-yl)-2- (((2R,7aS)-2- fluorotetrahydro- 1H-pyrrolizin- 7a(5H)- yl)methoxy) quinazoline | 562.1 (M + H)⁺ | ¹H NMR (400 MHz, DMSO-d6) δ ppm 10.65 − 10.82 (m, 1 H), 9.22 (br d, J = 11.1 Hz, 1 H), 8.93 − 9.09 (m, 1 H), 8.18 (d, J = 8.6 Hz, 1 H), 7.73 (d, J = 1.7 Hz, 1 H), 7.61 − 7.67 (m, 2 H), 7.51 (dd, J = 8.7, 1.8 Hz, 1 H), 5.49 − 5.68 (m, 1 H), 4.60 (s, 2 H), 4.47 (br d, J = 14.0 Hz, 2 H), 4.22 (br s, 2 H), 3.69 − 3.86 (m, 6 H), 2.55 (d, J = 0.6 Hz, 3 H), 1.95 − 2.34 (m, 9 H). |
| 40 | 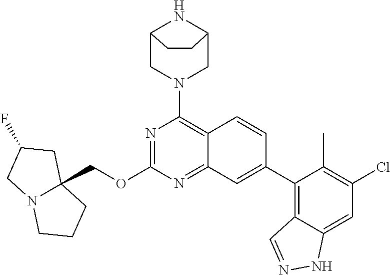 [SMILES: Cc1c(Cl)cc2[nH]ncc2c1-c1ccc2c(N3CC4CCC(C3)N4)nc(OC[C@@]34CCCN3C[C@H](F)C4)nc2c1] | 4-((1R,5S)-3,8- diazabicyclo[3.2. 1]octan-3-yl)-7- (6-chloro-5- methyl-1H- indazol-4-yl)-2- (((2R,7aS)-2- fluorotetrahydro- 1H-pyrrolizin- 7a(5H)- yl)methoxy) quinazoline | 562.1 (M + H)⁺ | ¹H NMR (400 MHz, DMSO-d₆) δ ppm 10.77 (br dd, J = 7.1, 2.9 Hz, 1 H), 9.23 (br d, J = 9.0 Hz, 1 H), 8.97 - 9.12 (m, 1 H), 8.17 (d, J = 8.8 Hz, 1 H), 7.78 (s, 1 H), 7.63 (dd, J = 18.8, 1.3 Hz, 2 H), 7.45 (dd, J = 8.6, 1.9 Hz, 1 H), 5.51 − 5.70 (m, 1 H), 4.60 (s, 2 H), 4.49 (br d, J = 13.0 Hz, 2 H), 4.22 (br s, 3 H), 3.74 (br dd, J = 13.6, 2.7 Hz, 4 H), 3.30 − 3.39 (m, 1 H), 2.31 (s, 3 H), 1.94 − 2.30 (m, 9 H). |
| 41 | 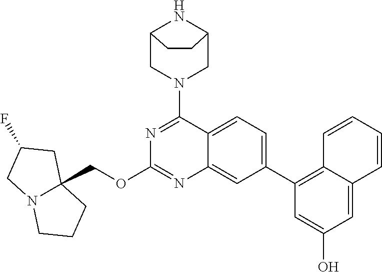 [SMILES: Oc1cc(-c2ccc3c(N4CC5CCC(C4)N5)nc(OC[C@@]45CCCN4C[C@H](F)C5)nc3c2)c2ccccc2c1] | 4-(4-((1R,5S)- 3,8- diazabicyclo[3.2. 1]octan-3-yl)-2- (((2R,7aS)-2- fluorotetrahydro- 1H-pyrrolizin- 7a(5H)- yl)methoxy) quinazolin-7- yl)naphthalen-2- ol | 540.4 (M + H)⁺ | ¹H NMR (400 MHz, METHANOL- d4) δ ppm 8.21 (d, J = 8.6 Hz, 1 H), 7.78 (s, 1 H), 7.78 (d, J = 6.8 Hz, 2 H), 7.70 (d, J = 8.6 Hz, 1 H), 7.65 (dd, J = 8.6, 1.7 Hz, 1 H), 7.45 (ddd, J = 8.2, 7.0, 1.1 Hz, 1 H), 7.23 − 7.28 (m, 2 H), 7.13 (d, J = 2.5 Hz, 1 H), 5.44 − 5.82 (m, 1 H), 4.87 − 4.93 (m, 1 H), 4.75 − 4.80 (m, 2 H), 4.30 (br s, 2 H), 3.87 − 4.05 (m, 5 H), 3.47 − 3.55 (m, 1 H), 2.59 − 2.81 (m, 2 H), 2.34 − 2.53 (m, 3 H), 2.15 − 2.30 (m, 5 H). |
| 42 | 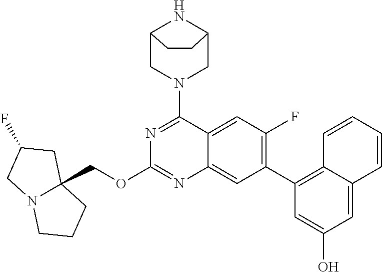 [SMILES: Oc1cc(-c2cc3nc(OC[C@@]45CCCN4C[C@H](F)C5)nc(N4CC5CCC(C4)N5)c3cc2F)c2ccccc2c1] | 4-(4-((1R,5S)-3,8- diazabicyclo[3.2. 1]octan-3-yl)-6- fluoro-2- (((2R,7aS)-2- fluorotetrahydro- 1H-pyrrolizin- 7a(5H)- yl)methoxy) quinazolin-7- yl)naphthalen-2-ol | 558.2 (M + H)⁺ | ¹H NMR (400 MHz, METHANOL- d4) δ ppm 7.89 (d, J = 10.2 Hz, 1 H), 7.75 − 7.79 (m, 2 H), 7.42 − 7.49 (m, 2 H), 7.22 − 7.30 (m, 2 H), 7.12 (d, J = 2.3 Hz, 1 H), 5.48 − 5.72 (m, 1 H), 4.68 − 4.80 (m, 4 H), 4.28 (br s, 2 H), 3.85 − 4.05 (m, 5 H), 3.46 − 3.54 (m, 1 H), 2.55 − 2.83 (m, 2 H), 2.33 − 2.50 (m, 3 H), 2.13 − 2.28 (m, 5 H). |
| 43 | 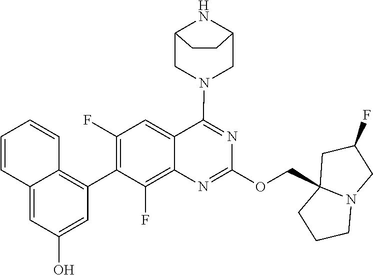 [SMILES: Oc1cc(-c2c(F)cc3c(N4CC5CCC(C4)N5)nc(OC[C@]45CCCN4C[C@H](F)C5)nc3c2F)c2ccccc2c1] | 4-(4-((1R,5S)- 3,8- diazabicyclo[3.2. 1]octan-3-yl)-6,8- difluoro-2- (((2R,7aR)-2- fluorotetrahydro- 1H-pyrrolizin- 7a(5H)- yl)methoxy) quinazolin-7- yl)naphthalen-2- ol | 576.2 (M + H)⁺ | ¹H NMR (400 MHz, METHANOL- d4) δ ppm 7.79 (br d, J = 8.4 Hz, 2 H), 7.43 − 7.50 (m, 1 H), 7.36 (d, J = 8.4 Hz, 1 H), 7.32 (d, J = 2.3 Hz, 1 H), 7.26 (d, J = 6.9 Hz, 1 H), 7.13 − 7.17 (m, 1 H), 5.40 − 5.61 (m, 1 H), 4.78 (br dd, J = 5.4, 3.6 Hz, 4 H), 4.27 (br s, 2 H), 3.99 − 4.15 (m, 1 H), 3.90 (br s, 2 H), 3.63 − 3.76 (m, 1 H), 3.45 − 3.62 (m, 2 H), 2.68 − 2.80 (m, 1 H), 2.37 − 2.59 (m, 2 H), 2.25 − 2.35 (m, 2 H), 2.19 (br s, 5 H). |
| 44 | 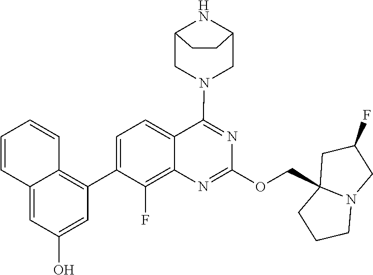 [SMILES: Oc1cc(-c2ccc3c(N4CC5CCC(C4)N5)nc(OC[C@]45CCCN4C[C@H](F)C5)nc3c2F)c2ccccc2c1] | 4-(4-((1R,5S)- 3,8- diazabicyclo[3.2. 1]octan-3-yl)-8- fluoro-2- (((2R,7aR)-2- fluorotetrahydro- 1H-pyrrolizin- 7a(5H)- yl)methoxy) quinazolin-7- yl)naphthalen-2- ol | 558.2 (M + H)⁺ | ¹H NMR (400 MHz, METHANOL- d4) δ ppm 7.94 − 8.00 (m, 1 H), 7.75 − 7.81 (m, 1 H), 7.40 − 7.51 (m, 3 H), 7.28 (d, J = 2.5 Hz, 2 H), 7.10 − 7.16 (m, 1 H), 5.41 − 5.64 (m, 1 H), 4.71 − 4.82 (m, 4 H), 4.24 − 4.32 (m, 2 H), 3.99 − 4.11 (m, 1 H), 3.84 − 3.97 (m, 2 H), 3.62 − 3.78 (m, 1 H), 3.43 − 3.61 (m, 2 H), 2.68 − 2.81 (m, 1 H), 2.40 − 2.59 (m, 2 H), 2.25 − 2.36 (m, 2 H), 2.19 (br s, 5 H). |
| 45 | 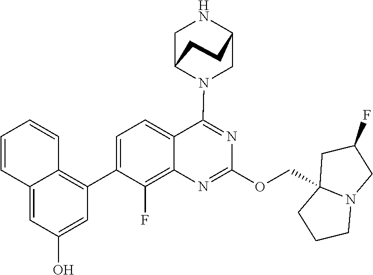 | 4-(4-((1S,4S)-2,5- diazabicyclo[2.2. 2]octan-2-yl)-8- fluoro-2- (((2R,7aS)-2- fluorotetrahydro- 1H-pyrrolizin- 7a(5H)- yl)methoxy) quinazolin-7- yl)naphthalen-2- ol | 558.2 (M + H)⁺ | ¹H NMR (400 MHz, METHANOL- d4) δ ppm 8.11 (d, J = 8.4 Hz, 1 H), 7.74 − 7.81 (m, 1 H), 7.40 − 7.50 (m, 3 H), 7.28 (d, J = 2.3 Hz, 1 H), 7.24 (dd, J = 7.1, 1.3 Hz, 1 H), 7.13 (br s, 1 H), 5.46 − 5.71 (m, 1 H), 5.18 − 5.26 (m, 1 H), 4.66 − 4.78 (m, 2 H), 4.51 − 4.61 (m, 1 H), 4.36 − 4.46 (m, 1 H), 3.80 − 4.11 (m, 5 H), 3.56 − 3.65 (m, 1 H), 3.43 − 3.55 (m, 1 H), 2.05 − 2.83 (m, 10 H). |
| 46 | 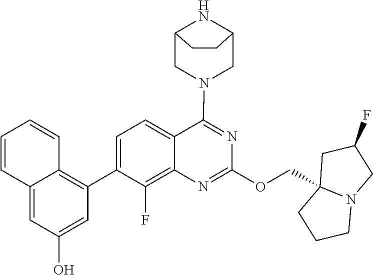 [SMILES: Oc1cc(-c2ccc3c(N4CC5CCC(C4)N5)nc(OC[C@@]45CCCN4C[C@H](F)C5)nc3c2F)c2ccccc2c1] | 4-(4-((1R,5S)- 3,8- diazabicyclo[3.2. 1]octan-3-yl)-8- fluoro-2- (((2R,7aS)-2- fluorotetrahydro- 1H-pyrrolizin- 7a(5H)- yl)methoxy) quinazolin-7- yl)naphthalen-2-ol | 558.2 (M + H)⁺ | ¹H NMR (400 MHz, METHANOL- d4) δ ppm 7.93 − 8.02 (m, 1 H), 7.74 − 7.81 (m, 1 H), 7.41 − 7.50 (m, 3 H), 7.26 − 7.30 (m, 1 H), 7.20 − 7.26 (m, 1 H), 7.08 − 7.15 (m, 1 H), 5.46 − 5.70 (m, 1 H), 4.65 − 4.81 (m, 4 H), 4.28 (br s, 2 H), 3.79 − 4.10 (m, 5 H), 3.41 − 3.55 (m, 1 H), 2.55 − 2.81 (m, 2 H), 2.31 − 2.51 (m, 3 H), 2.19 (br s, 5 H). |
| 47 | 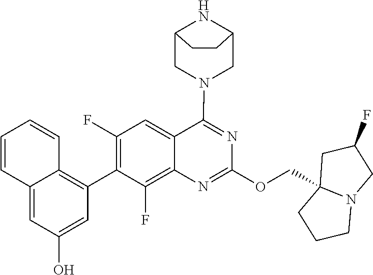 [SMILES: Oc1cc(-c2c(F)cc3c(N4CC5CCC(C4)N5)nc(OC[C@@]45CCCN4C[C@H](F)C5)nc3c2F)c2ccccc2c1] | 4-(4-((1R,5S)- 3,8- diazabicyclo[3.2. 1]octan-3-yl)-6,8- difluoro-2- (((2R,7aS)-2- fluorotetrahydro- 1H-pyrrolizin- 7a(5H)- yl)methoxy) quinazolin-7- yl)naphthalen-2-ol | 576.2 (M + H)⁺ | ¹H NMR (400 MHz, METHANOL- d4) δ ppm 7.71 − 7.82 (m, 2 H), 7.42 − 7.49 (m, 1 H), 7.30 − 7.38 (m, 2 H), 7.21 − 7.28 (m, 1 H), 7.12 − 7.17 (m, 1 H), 5.49 − 5.68 (m, 1 H), 4.71 (s, 4 H), 4.27 (br s, 2 H), 3.83 − 4.10 (m, 5 H), 3.44 − 3.53 (m, 1 H), 2.67 (s, 2 H), 2.31 − 2.51 (m, 3 H), 2.19 (s, 5 H). |
| 48 | 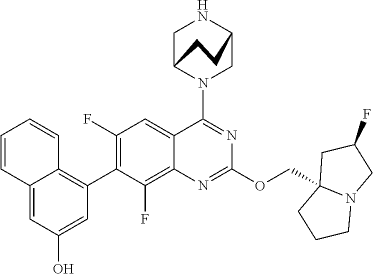 | 4-(4-((1S,4S)-2,5- diazabicyclo[2.2. 2]octan-2-yl)-6,8- difluoro-2- (((2R,7aS)-2- fluorotetrahydro- 1H-pyrrolizin- 7a(5H)- yl)methoxy) quinazolin-7- yl)naphthalen-2- ol | 576.2 (M + H)⁺ | ¹H NMR (400 MHz, METHANOL- d4) δ ppm 7.86 − 7.94 (m, 1 H), 7.79 (d, J = 8.2 Hz, 1 H), 7.45 (d, J = 7.9 Hz, 1 H), 7.30 − 7.38 (m, 2 H), 7.25 (d, J = 6.9 Hz, 1 H), 7.14 (t, J = 2.6 Hz, 1 H), 5.41 − 5.70 (m, 1 H), 5.12 − 5.22 (m, 1 H), 4.70 (t, J = 4.3 Hz, 2 H), 4.50 − 4.60 (m, 1 H), 4.36 − 4.47 (m, 1 H), 3.81 − 4.11 (m, 5 H), 3.54 − 3.67 (m, 1 H), 3.40 − 3.52 (m, 1 H), 2.08 − 2.79 (m, 10 H). |
| 49 | 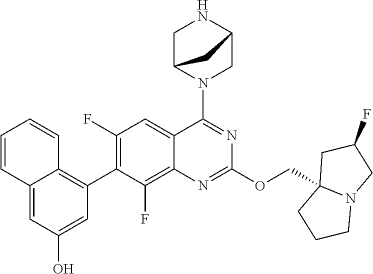 [SMILES: Oc1cc(-c2c(F)cc3c(N4C[C@@H]5C[C@H]4CN5)nc(OC[C@@]45CCCN4C[C@H](F)C5)nc3c2F)c2ccccc2c1] | 4-(4-((1S,4S)-2,5- diazabicyclo[2.2. 1]heptan-2-yl)- 6,8-difluoro-2- (((2R,7aS)-2- fluorotetrahydro- 1H-pyrrolizin- 7a(5H)- yl)methoxy) quinazolin-7- yl)naphthalen-2- ol | 562.2 (M + H)⁺ | ¹H NMR (400 MHz, METHANOL- d4) δ ppm 7.83 − 7.89 (m, 1 H) 7.77 − 7.82 (m, 1 H) 7.43 − 7.49 (m, 1 H) 7.29 − 7.37 (m, 2 H) 7.21 − 7.28 (m, 1 H) 7.11 − 7.17 (m, 1 H) 5.46 − 5.68 (m, 2 H) 4.69 (s, 3 H) 4.51 − 4.62 (m, 1 H) 4.18 − 4.28 (m, 1 H) 3.76 − 4.10 (m, 4 H) 3.54 − 3.65 (m, 1 H) 3.43 − 3.54 (m, 1 H) 2.53 − 2.77 (m, 2 H) 2.29 − 2.53 (m, 4 H) 2.09 − 2.28 (m, 2 H). |
| 50 | 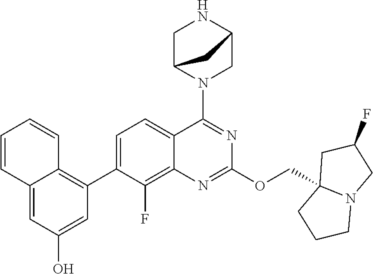 [SMILES: Oc1cc(-c2ccc3c(N4C[C@@H]5C[C@H]4CN5)nc(OC[C@@]45CCCN4C[C@H](F)C5)nc3c2F)c2ccccc2c1] | 4-(4-((1S,4S)-2,5- diazabicyclo[2.2. 1]heptan-2-yl)-8- fluoro-2- (((2R,7aS)-2- fluorotetrahydro- 1H-pyrrolizin- 7a(5H)- yl)methoxy) quinazolin-7- yl)naphthalen-2- ol | 544.1 (M + H)⁺ | ¹H NMR (400 MHz, METHANOL- d4) δ ppm 8.03 − 8.11 (m, 1 H) 7.75 − 7.82 (m, 1 H) 7.40 − 7.51 (m, 3 H) 7.26 − 7.31 (m, 1 H) 7.20 − 7.26 (m, 1 H) 7.09 − 7.16 (m, 1 H) 5.47 − 5.67 (m, 2 H) 4.65 − 4.80 (m, 3 H) 4.50 − 4.59 (m, 1 H) 4.22 − 4.30 (m, 1 H) 3.74 − 4.10 (m, 4 H) 3.56 − 3.66 (m, 1 H) 3.43 − 3.53 (m, 1 H) 2.54 − 2.76 (m, 2 H) 2.28 − 2.51 (m, 4 H) 2.11 − 2.25 (m, 2 H). |
| 51 | 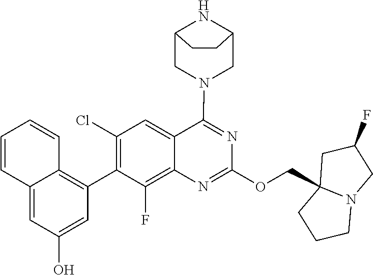 [SMILES: Oc1cc(-c2c(Cl)cc3c(N4CC5CCC(C4)N5)nc(OC[C@]45CCCN4C[C@H](F)C5)nc3c2F)c2ccccc2c1] | 4-(4-((1R,5S)- 3,8- diazabicyclo[3.2. 1]octan-3-yl)-6- chloro-8-fluoro- 2-(((2R,7aR)-2- fluorotetrahydro- 1H-pyrrolizin- 7a(5H)- yl)methoxy) quinazolin-7- yl)naphthalen-2- ol | 592.2 (M + H)⁺ | ¹H NMR (400 MHz, METHANOL- d4) δ ppm 8.05 (d, J = 1.5 Hz, 1 H), 7.79 (d, J = 8.4 Hz, 1 H), 7.45 (ddd, J = 8.3, 6.0, 2.1 Hz, 1 H), 7.31 (d, J = 2.5 Hz, 1 H), 7.19 − 7.26 (m, 2 H), 7.05 − 7.07 (m, 1 H), 5.43 − 5.61 (m, 1 H), 4.73 − 4.82 (m, 4 H), 4.28 (br s, 2 H), 4.05 (br d, J = 2.7 Hz, 1 H), 3.94 (dt, J = 13.8, 3.8 Hz, 2 H), 3.63 − 3.73 (m, 1 H), 3.42 − 3.62 (m, 2 H), 2.67 − 2.79 (m, 1 H), 2.39 − 2.56 (m, 2 H), 2.22 − 2.35 (m, 2 H), 2.17 (br s, 5 H). |
| 52 | 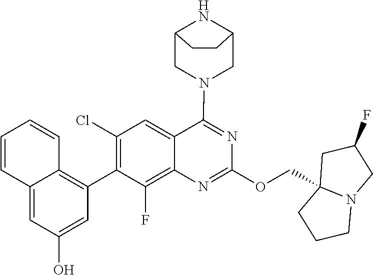 [SMILES: Oc1cc(-c2c(Cl)cc3c(N4CC5CCC(C4)N5)nc(OC[C@@]45CCCN4C[C@H](F)C5)nc3c2F)c2ccccc2c1] | 4-(4-((1R,5S)- 3,8- diazabicyclo[3.2. 1]octan-3-yl)-6- chloro-8-fluoro- 2-(((2R,7aS)-2- fluorotetrahydro- 1H-pyrrolizin- 7a(5H)- yl)methoxy) quinazolin-7- yl)naphthalen-2- ol | 592.2 (M + H)⁺ | ¹H NMR (400 MHz, METHANOL- d4) δ ppm 8.05 (d, J = 1.5 Hz, 1 H), 7.79 (d, J = 8.4 Hz, 1 H), 7.44 (ddd, J = 8.3, 5.7, 2.3 Hz, 1 H), 7.30 (d, J = 2.5 Hz, 1 H), 7.19 − 7.25 (m, 2 H), 7.05 (dd, J = 2.3, 1.3 Hz, 1 H), 5.46 − 5.71 (m, 1 H), 4.67 − 4.82 (m, 4 H), 4.28 (br s, 2 H), 3.80 − 4.12 (m, 5 H), 3.44 − 3.52 (m, 1 H), 2.55 - 2.81 (m, 2 H), 2.31 − 2.49 (m, 3 H), 2.11 − 2.24 (m, 5 H). |
| 53 | 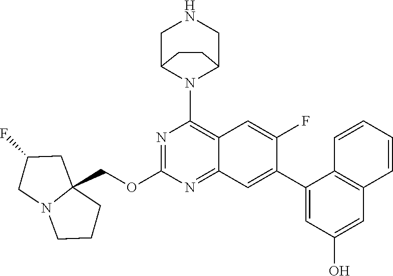 [SMILES: Oc1cc(-c2cc3nc(OC[C@@]45CCCN4C[C@H](F)C5)nc(N4C5CCC4CNC5)c3cc2F)c2ccccc2c1] | 4-(4-((1R,5S)- 3,8- diazabicyclo[3.2. 1]octan-8-yl)-6- fluoro-2- (((2R,7aS)-2- fluorotetrahydro- 1H-pyrrolizin- 7a(5H)- yl)methoxy) quinazolin-7- yl)naphthalen-2- ol | 558.2 (M + H)⁺ | ¹H NMR (400 MHz, METHANOL- d4) δ ppm 7.95 (d, J = 9.8 Hz, 1 H), 7.75 − 7.79 (m, 2 H), 7.42 − 7.48 (m, 2 H), 7.22 − 7.29 (m, 2 H), 7.12 (d, J = 2.5 Hz, 1 H), 5.47 − 5.73 (m, 1 H), 5.21 (br s, 2 H), 4.65 − 4.77 (m, 2 H), 3.87 − 4.04 (m, 3 H), 3.70 − 3.78 (m, 2 H), 3.43 − 3.54 (m, 3 H), 2.54 − 2.82 (m, 2 H), 2.30 − 2.49 (m, 5 H), 2.12 − 2.29 (m, 3 H). |
| 54 | 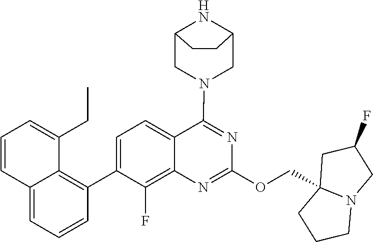 [SMILES: CCc1cccc2cccc(-c3ccc4c(N5CC6CCC(C5)N6)nc(OC[C@@]56CCCN5C[C@H](F)C6)nc4c3F)c12] | 4-((1R,5S)-3,8- diazabicyclo[3.2. 1]octan-3-yl)-7- (8- ethylnaphthalen- 1-yl)-8-fluoro-2- (((2R,7aS)-2- fluorotetrahydro- 1H-pyrrolizin- 7a(5H)- yl)methoxy) quinazoline | 570.1 (M + H)⁺ | ¹H NMR (400 MHz, METHANOL- d4) δ ppm 7.96 − 8.05 (m, 1 H), 7.86 (t, J = 7.9 Hz, 2 H), 7.46 − 7.57 (m, 2 H), 7.37 − 7.42 (m, 1 H), 7.32 − 7.37 (m, 1 H), 7.22 − 7.30 (m, 1 H), 5.24 − 5.47 (m, 1 H), 4.48 − 4.62 (m, 2 H), 4.35 (d, J = 10.5 Hz, 1 H), 4.24 − 4.31 (m, 1 H), 3.61 − 3.73 (m, 4 H), 3.20 − 3.34 (m, 3 H), 3.07 (td, J = 9.2, 6.0 Hz, 1 H), 2.47 − 2.57 (m, 2 H), 2.13 − 2.46 (m, 3 H), 2.01 − 2.10 (m, 2 H), 1.83 − 1.98 (m, 5 H), 0.93 (td, J = 7.4, 1.8 Hz, 3 H). |
| 55 | 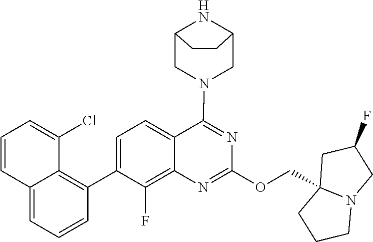 [SMILES: Fc1c(-c2cccc3cccc(Cl)c23)ccc2c(N3CC4CCC(C3)N4)nc(OC[C@@]34CCCN3C[C@H](F)C4)nc12] | 4-((1R,5S)-3,8- diazabicyclo[3.2. 1]octan-3-yl)-7- (8- chloronaphthalen- 1-yl)-8-fluoro-2- (((2R,7aS)-2- fluorotetrahydro- 1H-pyrrolizin- 7a(5H)- yl)methoxy) quinazoline | 576.2 (M + H)⁺ | ¹H NMR (400 MHz, METHANOL- d4) δ ppm 8.07 − 8.12 (m, 1 H), 8.06 − 8.12 (m, 1 H), 7.98 − 8.03 (m, 1 H), 7.83 (d, J = 8.6 Hz, 1 H), 7.56 − 7.71 (m, 2 H), 7.47 − 7.54 (m, 2 H), 7.28 (dd, J = 8.6, 6.9 Hz, 1 H), 5.19 − 5.53 (m, 1 H), 4.48 − 4.65 (m, 2 H), 4.21 − 4.40 (m, 2 H), 3.60 − 3.73 (m, 4 H), 3.24 − 3.34 (m, 2 H), 3.07 (td, J = 9.4, 6.0 Hz, 1 H), 2.14 − 2.46 (m, 4 H), 1.87 − 2.11 (m, 7 H). |
| 56 | 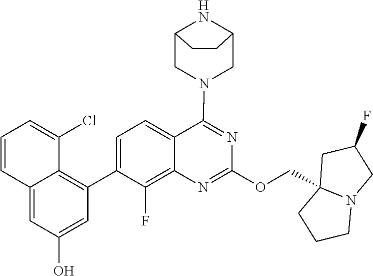 [SMILES: Oc1cc(-c2ccc3c(N4CC5CCC(C4)N5)nc(OC[C@@]45CCCN4C[C@H](F)C5)nc3c2F)c2c(Cl)cccc2c1] | 4-(4-((1R,5S)- 3,8- diazabicyclo[3.2. 1]octan-3-yl)-8- fluoro-2- (((2R,7aS)-2- fluorotetrahydro- 1H-pyrrolizin- 7a(5H)-yl)methoxy) quinazolin-7-yl)-5- chloronaphthalen- 2-ol | 592.0 (M + H)⁺ | ¹H NMR (400 MHz, METHANOL- d4) δ ppm 7.97 (d, J = 8.4 Hz, 1 H), 7.82 (dd, J = 8.0, 1.4 Hz, 1 H), 7.36 − 7.52 (m, 4 H), 7.12 (dd, J = 2.5, 0.8 Hz, 1 H), 5.51 − 5.75 (m, 1 H), 4.81 - 4.84 (m, 1 H), 4.77 − 4.80 (m, 2 H), 4.77 − 4.80 (m, 3 H), 4.30 − 4.38 (m, 2 H), 4.03 − 4.15 (m, 1 H), 3.86 − 4.02 (m, 4 H), 3.47 − 3.60 (m, 1 H), 2.35 − 2.86 (m, 6 H), 2.18 − 2.29 (m, 5 H). |
| 57 | 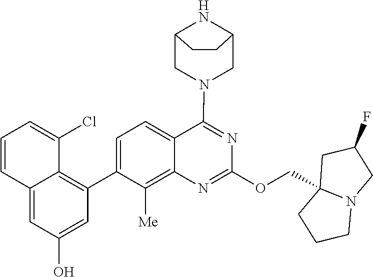 [SMILES: Cc1c(-c2cc(O)cc3cccc(Cl)c23)ccc2c(N3CC4CCC(C3)N4)nc(OC[C@@]34CCCN3C[C@H](F)C4)nc12] | 4-(4-((1R,5S)- 3,8- diazabicyclo[3.2. 1]octan-3-yl)-2- (((2R,7aS)-2- fluorotetrahydro- 1H-pyrrolizin- 7a(5H)- yl)methoxy)-8- methylquinazolin- 7-yl)-5- chloronaphthalen- 2-ol | 588.2 (M + H)⁺ | ¹H NMR (400 MHz, METHANOL- d4) δ ppm 7.89 (d, J = 8.6 Hz, 1 H), 7.75 (dd, J = 8.2, 1.0 Hz, 1 H), 7.27 − 7.38 (m, 4 H), 6.92 (d, J = 2.9 Hz, 1 H), 0.00 (dt, J = 51.4, 3.3 Hz, 1 H), 4.56 − 4.77 (m, 5 H), 4.27 (br s, 2 H), 3.90 − 4.09 (m, 3 H), 3.76 − 3.84 (m, 2 H), 3.47 − 3.56 (m, 1 H), 2.30 (s, 14 H). |
| 58 | 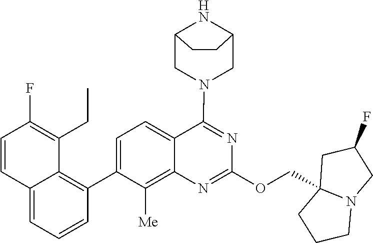 [SMILES: CCc1c(F)ccc2cccc(-c3ccc4c(N5CC6CCC(C5)N6)nc(OC[C@@]56CCCN5C[C@H](F)C6)nc4c3C)c12] | 4-((1R,5S)-3,8- diazabicyclo[3.2. 1]octan-3-yl)-7- (8-ethyl-7- fluoronaphthalen- 1-yl)-2- (((2R,7aS)-2- fluorotetrahydro- 1H-pyrrolizin- 7a(5H)- yl)methoxy)-8- methylquinazolin | 584.2 (M + H)⁺ | ¹H NMR (400 MHz, METHANOL- d4) δ ppm 7.90 − 8.02 (m, 3 H), 7.48 − 7.55 (m, 1 H), 7.43 − 7.48 (m, 1 H), 7.33 − 7.40 (m, 1 H), 7.21 − 7.26 (m, 1 H), 5.52 − 5.71 (m, 1 H), 4.68 − 4.78 (m, 2 H), 4.56 − 4.65 (m, 2 H), 4.24 − 4.32 (m, 2 H), 3.76 − 4.08 (m, 5 H), 3.47 − 3.56 (m, 1 H), 2.31 − 2.85 (m, 7 H), 2.17 − 2.31 (m, 8 H), 0.69 − 0.80 (m, 3 H). |
| 59 | 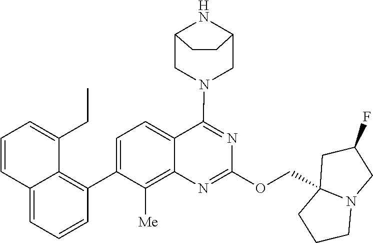 [SMILES: CCc1cccc2cccc(-c3ccc4c(N5CC6CCC(C5)N6)nc(OC[C@@]56CCCN5C[C@H](F)C6)nc4c3C)c12] | 4-((1R,5S)-3,8- diazabicyclo[3.2. 1]octan-3-yl)-7-(8- ethylnaphthalen- 1-yl)-2- (((2R,7aS)-2- fluorotetrahydro- 1H-pyrrolizin- 7a(5H)- yl)methoxy)-8- methylquinazoline | 566.4 (M + H)⁺ | ¹H NMR (400 MHz, METHANOL- d4) δ ppm 7.98 (dd, J = 8.4, 1.3 Hz, 1 H), 7.94 (d, J = 8.6 Hz, 1 H), 7.83 − 7.89 (m, 1 H), 7.35 − 7.56 (m, 4 H), 7.20 (dd, J = 7.0, 1.4 Hz, 1 H), 0.00 (qd, J = 52.0, 4.2 Hz, 1 H), 4.58 − 4.77 (m, 4 H), 4.28 (s, 2 H), 3.78 − 4.09 (m, 5 H), 3.46 − 3.55 (m, 1 H), 2.16 − 2.85 (m, 16 H), 0.86 (t, J = 7.3 Hz, 3 H). |
| 60 | 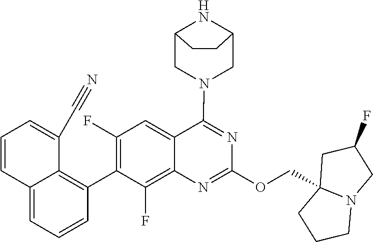 [SMILES: N#Cc1cccc2cccc(-c3c(F)cc4c(N5CC6CCC(C5)N6)nc(OC[C@@]56CCCN5C[C@H](F)C6)nc4c3F)c12] | 8-(4-((1R,5S)- 3,8- diazabicyclo[3.2. 1]octan-3-yl)-6,8- difluoro-2- (((2R,7aS)-2- fluorotetrahydro- 1H-pyrrolizin- 7a(5H)-yl)methoxy) quinazolin- 7-yl)-1- naphthonitrile | 585.2 (M + H)⁺ | ¹H NMR (400 MHz, METHANOL- d4) δ ppm 8.43 (dd, J = 8.6, 1.3 Hz, 1 H), 8.27 (dd, J = 8.3, 0.9 Hz, 1 H), 8.08 (dd, J = 7.2, 0.7 Hz, 1 H), 7.81 − 7.88 (m, 1 H), 7.71 − 7.76 (m, 3 H), 5.61 (td, J = 51.8, 4.0 Hz, 1 H), 4.64 − 4.74 (m, 4 H), 4.27 (br s, 2 H), 3.85 − 4.10 (m, 5 H), 3.44 − 3.54 (m, 1 H), 2.32 − 2.81 (m, 5 H), 2.19 (s, 5 H). |
| 61 | 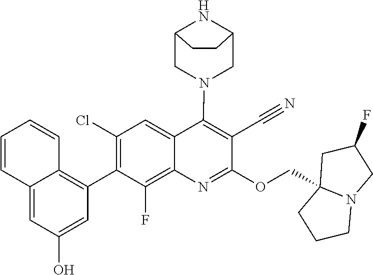 [SMILES: N#Cc1c(OC[C@@]23CCCN2C[C@H](F)C3)nc2c(F)c(-c3cc(O)cc4ccccc34)c(Cl)cc2c1N1CC2CCC(C1)N2] | 4-((1R,5S)-3,8- diazabicyclo[3.2. 1]octan-3-yl)-6- chloro-8-fluoro- 2-(((2R,7aS)-2- fluorotetrahydro- 1H-pyrrolizin- 7a(5H)- yl)methoxy)-7-(3- hydroxynaphthalen- 1-yl)quinoline-3- carbonitrile | 616.2 (M + H)⁺ | ¹H NMR (METHANOL-d4, 400 MHz) δ 8.23 (d, J = 1.7 Hz, 1 H), 7.75 (d, J = 8.4 Hz, 1H), 7.40 (ddd, J = 2.4, 5.6, 8.3 Hz, 1H), 7.26 (d, J = 2.5 Hz, 1H), 7.2-7.2 (m, 2 H), 7.03 (dd, J = 1.3, 2.3 Hz, 1 H), 5.2- 5.4 (m, 1 H), 4.3-4.4 (m, 2 H), 3.8- 3.9 (m, 2 H), 3.65 (br s, 2 H), 3.5- 3.6 (m, 2 H), 3.41 (td, J = 1.5, 12.5 Hz, 1 H), 3.1-3.3 (m, 2 H), 3.03 (dt, J = 6.0, 9.2 Hz, 1 H), 1.9-2.4 (m, 10 H). |
| 62 | 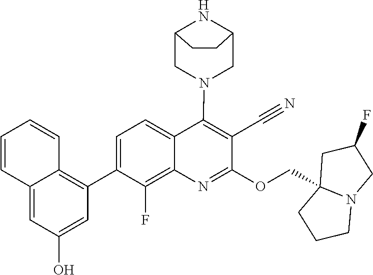 [SMILES: N#Cc1c(OC[C@@]23CCCN2C[C@H](F)C3)nc2c(F)c(-c3cc(O)cc4ccccc34)ccc2c1N1CC2CCC(C1)N2] | 4-((1R,5S)-3,8- diazabicyclo[3.2. 1]octan-3-yl)-8- fluoro-2- (((2R,7aS)-2- fluorotetrahydro- 1H-pyrrolizin- 7a(5H)- yl)methoxy)-7-(3- hydroxynaphthalen- 1-yl)quinoline-3- carbonitrile | 582.2 (M + H)⁺ | ¹H NMR (400 MHz, METHANOL- d4, δ ppm 8.16 (d, J = 8.2 Hz, 1 H), 7.78 (d, J = 8.4 Hz, 1 H), 7.64 (dd, J = 8.7, 6.6 Hz, 1 H), 7.37 − 7.50 (m, 2 H), 7.28 (d, J = 2.3 Hz, 1 H), 7.22 (ddd, J = 8.4, 6.9, 1.0 Hz, 1 H), 7.13 (d, J = 2.5 Hz, 1 H), 5.54 − 5.76 (m, 1 H), 4.84 (br d, J = 1.9 Hz, 1 H), 4.73 − 4.79 (m, 1 H), 4.33 (br d, J = 1.5 Hz, 2 H), 3.90 − 4.22 (m, 5 H), 3.81 (br dd, J = 11.3, 5.4 Hz, 2 H), 3.47 − 3.60 (m, 1 H), 2.23 − 2.88 (m, 10 H). |
| 63 | 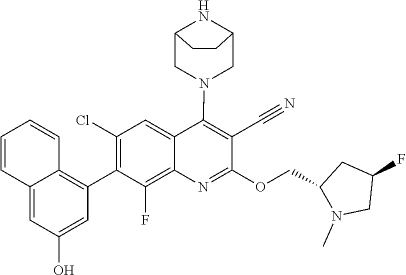 [SMILES: CN1C[C@H](F)C[C@H]1COc1nc2c(F)c(-c3cc(O)cc4ccccc34)c(Cl)cc2c(N2CC3CCC(C2)N3)c1C#N] | 4-((1R,5S)-3,8- diazabicyclo[3.2. 1]octan-3-yl)-6- chloro-8-fluoro- 2-(((2S,4R)-4- fluoro-1- methylpyrrolidin- 2-yl)methoxy)-7-(3- hydroxynaphthalen- 1-yl)quinoline-3- carbonitrile | 589.75 (M + H)⁺ | ¹H NMR (METHANOL-d4, 400 MHz) δ 8.22 (d, J = 1.5 Hz, 1 H), 7.74 (d, J = 8.2 Hz, 1 H), 7.40 (ddd, J = 1.8, 6.3, 8.2 Hz, 1 H), 7.27 (d, J = 2.5 Hz, 1 H), 7.1-7.2 (m, 2 H), 7.04 (d, J = 2.5 Hz, 1 H), 5.1-5.3 (m, 1 H), 4.7-4.7 (m, 1 H), 4.46 (ddd, J = 1.4, 5.6, 11.4 Hz, 1 H), 3.8-3.9 (m, 2 H), 3.67 (br s, 2 H), 3.4-3.6 (m, 3 H), 3.1-3.2 (m, 1 H), 2.6-2.8 (m, 1 H), 2.58 (s, 3 H), 2.2-2.4 (m, 3 H), 1.9-2.1 (m, 3 H). |
| 64 | 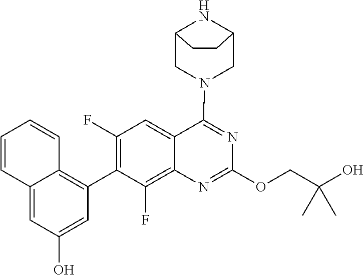 [SMILES: CC(C)(O)COc1nc(N2CC3CCC(C2)N3)c2cc(F)c(-c3cc(O)cc4ccccc34)c(F)c2n1] | 4-(4-((1R,5S)- 3,8- diazabicyclo[3.2. 1]octan-3-yl)-6,8- difluoro-2-(2- hydroxy-2- methylpropoxy) quinazolin-7- yl)naphthalen-2- ol | 506.9 (M + H)⁺ | ¹H NMR (CHLOROFORM-d, 400 MHz) δ 7.74 (d, J = 8.4 Hz, 1 H), 7.6-7.7 (m, 1 H), 7.40 (dt, J = 1.0, 7.5 Hz, 1 H), 7.35 (br d, J = 8.4 Hz, 1 H), 7.27 (d, J = 2.3 Hz, 1 H), 7.20 (ddd, J = 1.0, 7.0, 8.3 Hz, 1 H), 7.12 (d, J = 2.3 Hz, 1 H), 4.45 (br t, J = 11.2 Hz, 2 H), 4.27 (s, 2 H), 3.5- 3.7 (m, 4 H), 1.83 (s, 4 H), 1.33 (s, 6 H). |
| 65 | 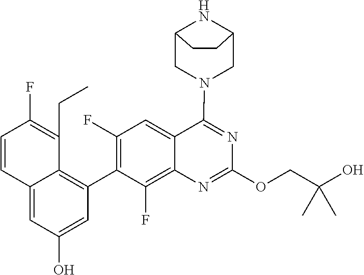 [SMILES: CCc1c(F)ccc2cc(O)cc(-c3c(F)cc4c(N5CC6CCC(C5)N6)nc(OCC(C)(C)O)nc4c3F)c12] | 4-(4-((1R,5S)- 3,8- diazabicyclo[3.2. 1]octan-3-yl)-6,8- difluoro-2-(2- hydroxy-2- methylpropoxy) quinazolin-7-yl)-5- ethyl-6- fluoronaphthalen- 2-ol | 553.2 (M + H)⁺ | ¹H NMR (METHANOL-d4, 400 MHz) δ 7.67 (dd, 1H, J = 5.9, 9.2 Hz), 7.61 (dd, 1H, J = 1.7, 10.0 Hz), 7.29 (d, 1H, J = 2.7 Hz), 7.24 (t, 1H, J = 9.4 Hz), 6.98 (d, 1H, J = 2.5 Hz), 4.4-4.5 (m, 2H), 4.27 (s, 2H), 3.6- 3.7 (m, 4H), 2.3-2.7 (m, 2H), 1.86 (br s, 4H), 1.33 (s, 6H), 0.81 (t, 3H, J = 7.4 Hz). |
| 66 | 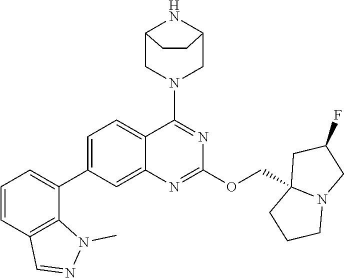 [SMILES: Cn1ncc2cccc(-c3ccc4c(N5CC6CCC(C5)N6)nc(OC[C@@]56CCCN5C[C@H](F)C6)nc4c3)c21] | 4-((1R,5S)-3,8- diazabicyclo [3.2. 1]octan-3-yl)-2- (((2R,7aS)-2- fluorotetrahydro- 1H-pyrrolizin- 7a(5H)- yl)methoxy)-7- (1-methyl-1H- indazol-7- yl)quinazoline | 528.2 (M + H)⁺ | ¹H NMR (400 MHz, DMSO-d6) δ ppm 8.20 (s, 1 H), 8.12 (d, J = 8.8 Hz, 1 H), 7.88 (dd, J = 7.7, 1.5 Hz, 1 H), 7.71 (d, J = 1.7 Hz, 1 H), 7.53 (dd, J = 8.6, 1.7 Hz, 1 H), 7.22 − 7.32 (m, 2 H), 5.50 − 5.69 (m, 1 H), 4.60 (s, 2 H), 4.48 (br d, J = 14.6 Hz, 2 H), 4.21 (br s, 3 H), 3.70 − 3.88 (m, 7 H), 3.63 (s, 3 H), 3.34 (br dd, J = 12.0, 6.4 Hz, 1 H), 1.96 − 2.33 (m, 8 H). |
| 67 | 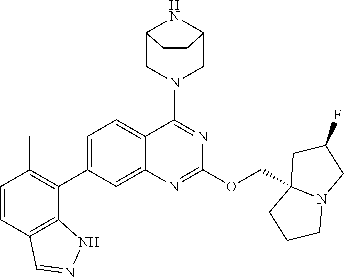 [SMILES: Cc1ccc2cn[nH]c2c1-c1ccc2c(N3CC4CCC(C3)N4)nc(OC[C@@]34CCCN3C[C@H](F)C4)nc2c1] | 4-((1R,5S)-3,8- diazabicyclo[3.2. 1]octan-3-yl)-2- (((2R,7aS)-2- fluorotetrahydro- 1H-pyrrolizin- 7a(5H)- yl)methoxy)-7- (6-methyl-1H- indazol-7- yl)quinazoline | 528.2 (M + H)⁺ | ¹H NMR (400 MHz, DMSO-d6) δ ppm 8.15 (d, J = 8.6 Hz, 1 H), 8.10 (s, 1 H), 7.72 (d, J = 8.2 Hz, 1 H), 7.64 − 7.66 (m, 1 H), 7.43 (dd, J = 8.6, 1.9 Hz, 1 H), 7.14 (d, J = 8.4 Hz, 1 H), 5.48 − 5.70 (m, 1 H), 4.60 (s, 2 H), 4.47 (br d, J = 13.8 Hz, 3 H), 4.23 (br s, 3 H), 3.72 − 3.89 (m, 6 H), 3.31 − 3.39 (m, 1 H), 2.43 (s, 2 H), 2.31 (s, 6 H), 2.01 (br s, 4 H). |
| 68 | 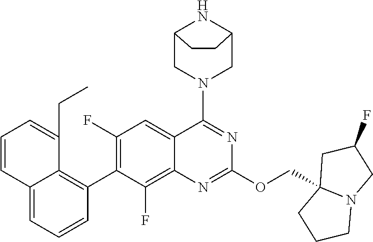 [SMILES: CCc1cccc2cccc(-c3c(F)cc4c(N5CC6CCC(C5)N6)nc(OC[C@@]56CCCN5C[C@H](F)C6)nc4c3F)c12] | 4-((1R,5S)-3,8- diazabicyclo[3.2. 1]octan-3-yl)-7- (8- ethylnaphthalen- 1-yl)-6,8- difluoro-2- (((2R,7aS)-2- fluorotetrahydro- 1H-pyrrolizin- 7a(5H)- yl)methoxy) quinazoline | 587.9 (M + H)⁺ | ¹H NMR (400 MHz, METHANOL- d4) δ ppm 8.04 − 8.10 (m, 1 H), 7.90 (dd, J = 8.0, 0.9 Hz, 1 H), 7.71 (dd, J = 9.7, 1.4 Hz, 1 H), 7.58 (dd, J = 8.2, 7.1 Hz, 1 H), 7.47 − 7.54 (m, 1 H), 7.33 − 7.44 (m, 2 H), 5.48 − 5.68 (m, 1 H), 4.70 (s, 4 H), 4.23 − 4.35 (m, 2 H), 3.77 − 4.09 (m, 5 H), 3.45 − 3.58 (m, 1 H), 2.30 − 2.80 (m, 7 H), 2.12 − 2.29 (m, 5 H), 0.95 (td, J = 7.4, 1.7 Hz, 3 H). |
| 69 | 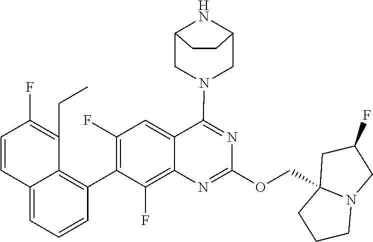 [SMILES: CCc1c(F)ccc2cccc(-c3c(F)cc4c(N5CC6CCC(C5)N6)nc(OC[C@@]56CCCN5C[C@H](F)C6)nc4c3F)c12] | 4-((1R,5S)-3,8- diazabicyclo[3.2. 1]octan-3-yl)-7- (8-ethyl-7- fluoronaphthalen- 1-yl)-6,8- difluoro-2- (((2R,7aS)-2- fluorotetrahydro- 1H-pyrrolizin- 7a(5H)- yl)methoxy) quinazoline | 605.9 (M + H)⁺ | ¹H NMR (400 MHz, METHANOL- d4) δ ppm 8.04 − 8.13 (m, 1 H) 7.96 (dd, J = 8.99, 6.06 Hz, 1 H) 7.73 (dd, J = 9.82, 1.46 Hz, 1 H) 7.53 − 7.62 (m, 1 H) 7.41 (d, J = 8.99 Hz, 2 H) 5.40 − 5.73 (m, 1 H) 4.70 (s, 4 H) 4.22 − 4.34 (m, 2 H) 3.80 − 4.12 (m, 5 H) 3.43 − 3.54 (m, 1 H) 2.30 − 2.82 (m, 7 H) 2.20 (br s, 5 H) 0.84 (br d, J = 1.88 Hz, 3 H). |
| 70 | 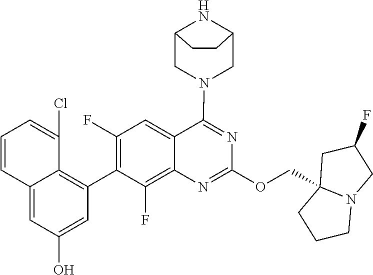 [SMILES: Oc1cc(-c2c(F)cc3c(N4CC5CCC(C4)N5)nc(OC[C@@]45CCCN4C[C@H](F)C5)nc3c2F)c2c(Cl)cccc2c1] | 4-(4-((1R,5S)- 3,8- diazabicyclo[3.2. 1]octan-3-yl)-6,8- difluoro-2- (((2R,7aS)-2- fluorotetrahydro- 1H-pyrrolizin- 7a(5H)-yl)methoxy) quinazolin- 7-yl)-5- chloronaphthalen- 2-ol | 610.2 (M + H)⁺ | ¹H NMR (400 MHz, METHANOL- d₄) δ ppm 7.74 − 7.81 (m, 1 H) 7.62 − 7.69 (m, 1 H) 7.37 (s, 3 H) 7.07 − 7.13 (m, 1 H) 5.47 − 5.71 (m, 1 H) 4.60 − 4.77 (m, 4 H) 4.21 − 4.34 (m, 2 H) 3.91 (br d, J = 15.47 Hz, 5 H) 3.45 − 3.54 (m, 1 H) 2.53 − 2.82 (m, 2 H) 2.30 − 2.51 (m, 3 H) 2.18 (br s, 5 H). |
| 71 | 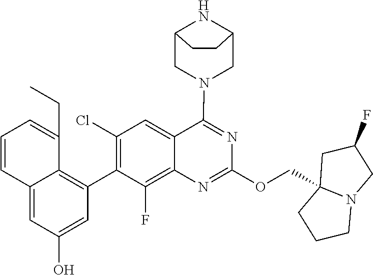 | 4-((1R,5S)-3,8- diazabicyclo[3.2. 1]octan-3-yl)-6- chloro-7-(8- ethylnaphthalen- 1-yl)-8-fluoro-2- (((2R,7aS)-2- fluorotetrahydro- 1H-pyrrolizin- 7a(5H)- yl)methoxy) quinazoline | 605.9 (M + H)⁺ | ¹H NMR (400 MHz, METHANOL- d4) δ ppm 8.01 − 8.08 (m, 1 H), 7.81 − 7.91 (m, 1 H), 7.50 (m, 2 H), 7.38 − 7.43 (m, 2 H), 7.27 (m, J = 6.7 Hz, 1 H), 5.62 − 5.66 (m, 1 H), 5.48 − 5.54 (m, 1 H), 4.28 (m, 2 H), 3.93 (m, J = 16.7 Hz, 7 H), 3.46 − 3.55 (m, 2 H), 2.29 − 2.48 (m, 7 H), 2.20 (m, 5 H), 0.97 (t, J = 6.9 Hz, 3 H). |
| 72 | 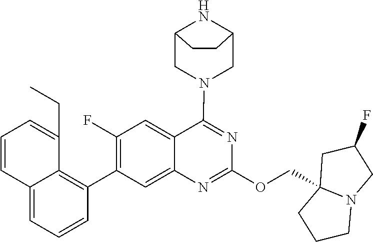 [SMILES: CCc1cccc2cccc(-c3cc4nc(OC[C@@]56CCCN5C[C@H](F)C6)nc(N5CC6CCC(C5)N6)c4cc3F)c12] | 4-((1R,5S)-3,8- diazabicyclo[3.2. 1]octan-3-yl)-7- (8- ethylnaphthalen- 1-yl)-6-fluoro-2- (((2R,7aS)-2- fluorotetrahydro- 1H-pyrrolizin- 7a(5H)- yl)methoxy) quinazoline | 570.2 (M + H)⁺ | ¹H NMR (400 MHz, METHANOL- d4) δ ppm 7.99 − 8.07 (m, 1 H), 7.87 (d, J = 7.3 Hz, 1 H), 7.74 − 7.83 (m, 2 H), 7.46 − 7.64 (m, 2 H), 7.41 (d, J = 6.7 Hz, 1 H), 7.33 (dd, J = 7.1, 1.3 Hz, 1 H), 5.48 − 5.73 (m, 1 H), 4.69 (m, J = 2.7, 2.7 Hz, 4 H), 4.28 (br s, 2 H), 3.79 − 4.09 (m, 5 H), 3.46 − 3.55 (m, 1 H), 2.34 − 2.82 (m, 7 H), 2.12 − 2.29 (m, 5 H), 0.91 (td, J = 7.4, 1.3 Hz, 3 H). |
| 73 | 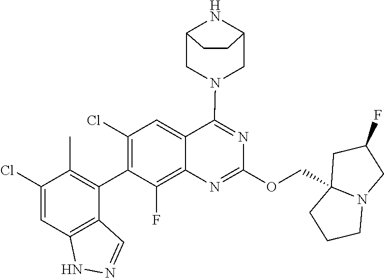 [SMILES: Cc1c(Cl)cc2[nH]ncc2c1-c1c(Cl)cc2c(N3CC4CCC(C3)N4)nc(OC[C@@]34CCCN3C[C@H](F)C4)nc2c1F] | 4-((1R,5S)-3,8- diazabicyclo[3.2. 1]octan-3-yl)-6- chloro-7-(6- chloro-5-methyl- 1H-indazol-4-yl)- 8-fluoro-2- (((2R,7aS)-2- fluorotetrahydro- 1H-pyrrolizin- 7a(5H)- yl)methoxy) quinazoline | 614.2 (M + H)⁺ | ¹H NMR (400 MHz, METHANOL- d4) δ ppm 8.08 (d, 1 H), 8.02 (s, 1H), 7.82 (s, 1 H), 7.52 (s, 1 H), 5.50 − 5.70 (m, 1 H), 4.70-4.78 (m, 4 H), 4.25 − 4.30 (m, 2 H), 3.81 − 4.00 (m, 5 H), 3.40- 3.55 (m, 1 H), 2.50 − 2.55 (m, 2 H), 2.30-2.45 (m, 3 H), 2.10-2.25 (m, 8 H). |
| 74 | 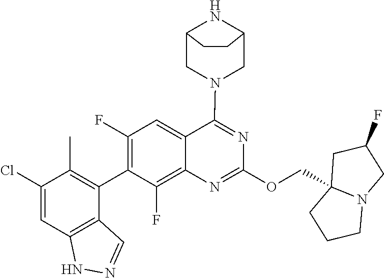 [SMILES: Cc1c(Cl)cc2[nH]ncc2c1-c1c(F)cc2c(N3CC4CCC(C3)N4)nc(OC[C@@]34CCCN3C[C@H](F)C4)nc2c1F] | 4-((1R,5S)-3,8- diazabicyclo[3.2. 1]octan-3-yl)-7- (6-chloro-5- methyl-1H- indazol-4-yl)-6,8- difluoro-2- (((2R,7aS)-2- fluorotetrahydro- 1H-pyrrolizin- 7a(5H)- yl)methoxy) quinazoline | 598.2 (M + H)⁺ | ¹H NMR (500 MHz, DMSO-d6) δ ppm 8.08 − 8.21 (m, 1 H) 7.84 (s, 1 H) 7.65 − 7.77 (m, 2 H) 5.12 − 5.36 (m, 1 H) 4.22 − 4.38 (m, 2 H) 4.04 − 4.14 (m, 1 H) 3.93 − 4.04 (m, 1 H) 3.47 − 3.53 (m, 2 H) 3.23 − 3.38 (m, 1 H) 2.93 − 3.15 (m, 3 H) 2.77 − 2.86 (m, 1 H) 2.23 (s, 3 H) 1.94 − 2.17 (m, 4 H) 1.63 − 1.88 (m, 8 H). |
| 75 | 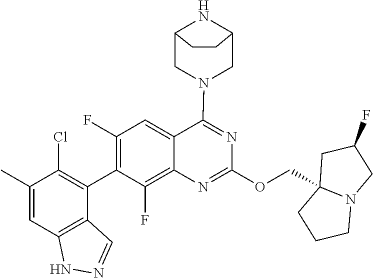 [SMILES: Cc1cc2[nH]ncc2c(-c2c(F)cc3c(N4CC5CCC(C4)N5)nc(OC[C@@]45CCCN4C[C@H](F)C5)nc3c2F)c1Cl] | 4-((1R,5S)-3,8- diazabicyclo[3.2. 1]octan-3-yl)-7- (5-chloro-6- methyl-1H- indazol-4-yl)-6,8- difluoro-2- (((2R,7aS)-2- fluorotetrahydro- 1H-pyrrolizin- 7a(5H)- yl)methoxy) quinazoline | 598.2 (M + H)⁺ | ¹H NMR (400 MHz, METHANOL- d4) δ ppm 8.51 − 8.59 (m, 1 H) 7.61 − 7.75 (m, 3 H) 5.28 − 5.49 (m, 1 H) 4.50 − 4.64 (m, 2 H) 4.29 − 4.46 (m, 2 H) 3.78 − 3.87 (m, 2 H) 3.64 − 3.73 (m, 2 H) 3.37 − 3.57 (m, 3 H) 3.09 − 3.22 (m, 1 H) 2.62 (s, 3 H) 2.16 − 2.52 (m, 3 H) 2.04 − 2.15 (m, 2 H) 1.97 (br s, 5 H). |
| 76 | 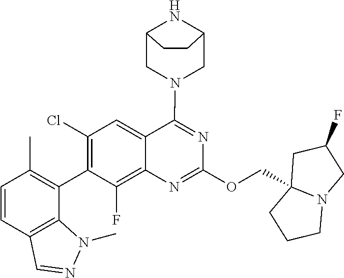 [SMILES: Cc1ccc2cnn(C)c2c1-c1c(Cl)cc2c(N3CC4CCC(C3)N4)nc(OC[C@@]34CCCN3C[C@H](F)C4)nc2c1F] | 4-((1R,5S)-3,8- diazabicyclo[3.2. 1]octan-3-yl)-6- chloro-7-(1,6- dimethyl-1H- indazol-7-yl)-8- fluoro-2- (((2R,7aS)-2- fluorotetrahydro- 1H-pyrrolizin- 7a(5H)- yl)methoxy) quinazoline | 593.8 (M + H)⁺ | ¹H NMR (400 MHz, METHANOL- d4) δ ppm 8.08 (d, 1 H), 8.02 (s, 1H), 7.79 − 7.81 (m, 1 H), 7.20 − 7.22 (m, 1 H), 5.49 − 5.75 (m, 1 H), 4.69-4.74 (m, 4 H), 4.22 − 4.32 (m, 2 H), 3.81 − 4.00 (m, 5 H), 3.47- 3.49 (m, 4 H), 2.54 − 2.69 (m, 2 H), 2.27-2.48 (m, 3 H), 2.12-2.22 (m, 8 H). |
| 77 | 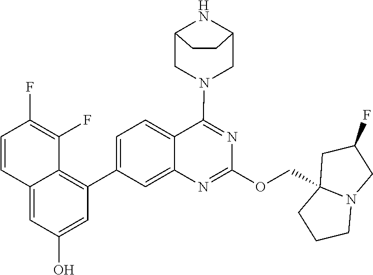 [SMILES: Oc1cc(-c2ccc3c(N4CC5CCC(C4)N5)nc(OC[C@@]45CCCN4C[C@H](F)C5)nc3c2)c2c(F)c(F)ccc2c1] | 4-(4-((1R,5S)- 3,8- diazabicyclo[3.2. 1]octan-3-yl)-2- (((2R,7aS)-2- fluorotetrahydro- 1H-pyrrolizin- 7a(5H)- yl)methoxy)quina zolin-7-yl)-5,6- difluoronaphthalen- 2-ol | 576.2 (M + H)⁺ | ¹H NMR (400 MHz, DMSO-d6) δ ppm 8.05 (d, J = 8.7 Hz, 1 H), 7.73 (dd, J = 8.0, 5.3 Hz, 1 H), 7.64 (s, 1 H), 7.52 − 7.62 (m, 1 H), 7.42 − 7.48 (m, 1 H), 7.34 (t, J = 2.0 Hz, 1 H), 7.05 (d, J = 2.3 Hz, 1 H), 5.48 − 5.72 (m, 1 H), 4.59 (s, 2 H), 4.42 − 4.50 (m, 2 H), 4.21 (br s, 2 H), 3.84 (br s, 3 H), 3.69 − 3.75 (m, 5 H), 1.94 − 2.34 (m, 10 H). |
| 78 | 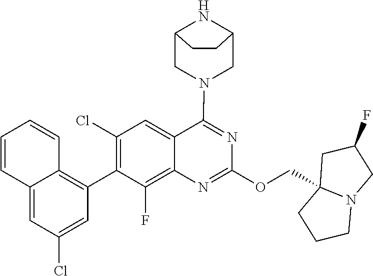 [SMILES: Fc1c(-c2cc(Cl)cc3ccccc23)c(Cl)cc2c(N3CC4CCC(C3)N4)nc(OC[C@@]34CCCN3C[C@H](F)C4)nc12] | 4-((1R,5S)-3,8- diazabicyclo[3.2. 1]octan-3-yl)-6- chloro-7-(3- chloronaphthalen- 1-yl)-8-fluoro-2- (((2R,7aS)-2- fluorotetrahydro- 1H-pyrrolizin- 7a(5H)- yl)methoxy) quinazoline | 610.2 (M + H)⁺ | ¹H NMR (METHANOL-d4, 400 MHz) δ 8.06 (d, 1H, J = 1.9 Hz), 7.9- 8.0 (m, 2H), 7.5-7.6 (m, 1H), 7.4- 7.5 (m, 2H), 7.4-7.4 (m, 1H), 5.2- 5.4 (m, 1H), 4.5-4.6 (m, 2H), 4.2- 4.3 (m, 1H), 4.2-4.2 (m, 1H), 3.6- 3.7 (m, 4H), 3.1-3.3 (m, 3H), 3.00 (dt, 1H, J = 5.9, 9.4 Hz), 2.2-2.4 (m, 2H), 2.1-2.2 (m, 1H), 1.9-2.0 (m, 2H), 1.8-1.9 (m, 5H). |
| 79 | 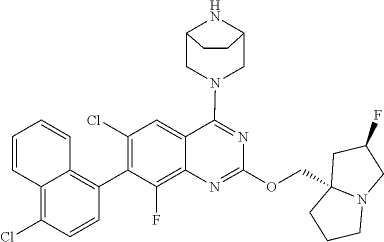 [SMILES: Fc1c(-c2ccc(Cl)c3ccccc23)c(Cl)cc2c(N3CC4CCC(C3)N4)nc(OC[C@@]34CCCN3C[C@H](F)C4)nc12] | 4-((1R,5S)-3,8- diazabicyclo[3.2. 1]octan-3-yl)-6- chloro-7-(4- chloronaphthalen- 1-yl)-8-fluoro-2- (((2R,7aS)-2- fluorotetrahydro- 1H-pyrrolizin- 7a(5H)- yl)methoxy) quinazoline | 610.2 (M + H)⁺ | ¹H NMR (METHANOL-d4, 400 MHz) δ 8.37 (d, 1H, J = 8.4 Hz), 7.95 (d, 1H, J = 1.7 Hz), 7.75 (d, 1H, J = 7.7 Hz), 7.67 (ddd, 1H, J = 1.1, 7.0, 8.4 Hz), 7.5-7.6 (m, 1H), 7.4- 7.5 (m, 2H), 5.2-5.4 (m, 1H), 4.4- 4.6 (m, 2H), 4.1-4.3 (m, 2H), 3.5- 3.7 (m, 4H), 3.1-3.3 (m, 3H), 2.99 (dt, 1H, J = 5.7, 9.5 Hz), 2.1-2.4 (m, 3H), 1.8-2.0 (m, 7H). |
| 80 | 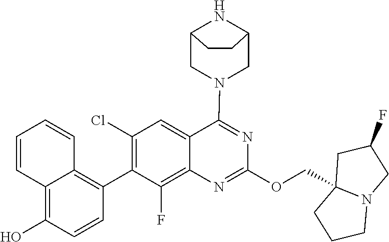 [SMILES: Oc1ccc(-c2c(Cl)cc3c(N4CC5CCC(C4)N5)nc(OC[C@@]45CCCN4C[C@H](F)C5)nc3c2F)c2ccccc12] | 4-(4-((1R,5S)- 3,8- diazabicyclo[3.2. 1]octan-3-yl)-6- chloro-8-fluoro- 2-(((2R,7aS)-2- fluorotetrahydro- 1H-pyrrolizin- 7a(5H)- yl)methoxy) quinazolin-7- yl)naphthalen-1- ol | 592.2 (M + H)⁺ | ¹H NMR (METHANOL-d4, 400 MHz) δ 8.3-8.4 (m, 1H), 7.91 (d, 1H, J = 1.5 Hz), 7.3-7.5 (m, 2H), 7.27 (d, 1H, J = 8.4 Hz), 7.20 (dd, 1H, J = 1.5, 7.7 Hz), 6.93 (d, 1H, J = 7.7 Hz), 5.2-5.4 (m, 1H), 4.4-4.5 (m, 2H), 4.1-4.3 (m, 2H), 3.6-3.7 (m, 4H), 3.1-3.3 (m, 3H), 3.00 (dt, 1H, J = 5.7, 9.4 Hz), 2.1-2.4 (m, 3H), 1.8-2.0 (m, 7H). |
| 81 | 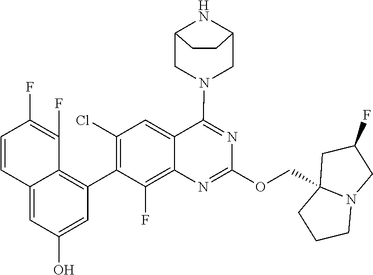 [SMILES: Oc1cc(-c2c(Cl)cc3c(N4CC5CCC(C4)N5)nc(OC[C@@]45CCCN4C[C@H](F)C5)nc3c2F)c2c(F)c(F)ccc2c1] | 4-(4-((1R,5S)- 3,8- diazabicyclo[3.2. 1]octan-3-yl)-6- chloro-8-fluoro- 2-(((2R,7aS)-2- fluorotetrahydro- 1H-pyrrolizin- 7a(5H)- yl)methoxy) quinazolin- 7-yl)-5,6- difluoronaphthalen- 2-ol | 616.2 (M + H)⁺ | ¹H NMR (400 MHz, METHANOL- d4) δ ppm 8.00 (d, J = 1.5 Hz, 1 H), 7.64 (ddd, J = 9.2, 4.8, 1.5 Hz, 1 H), 7.33 − 7.45 (m, 2 H), 7.06 − 7.09 (m, 1 H), 5.45 − 5.72 (m, 1 H), 4.67 − 4.80 (m, 4 H), 4.27 (br s, 2 H), 3.79 - 4.13 (m, 5 H), 3.44 − 3.53 (m, 1 H), 2.55 − 2.79 (m, 2 H), 2.31 − 2.50 (m, 3 H), 2.05 − 2.25 (m, 5 H). |
| 82 | 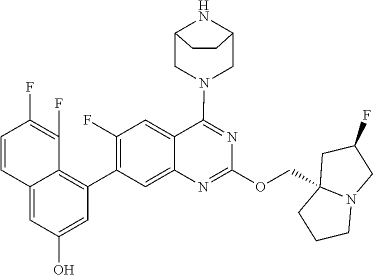 [SMILES: Oc1cc(-c2cc3nc(OC[C@@]45CCCN4C[C@H](F)C5)nc(N4CC5CCC(C4)N5)c3cc2F)c2c(F)c(F)ccc2c1] | 4-(4-((1R,5S)- 3,8- diazabicyclo[3.2. 1]octan-3-yl)-6- fluoro-2- (((2R,7aS)-2- fluorotetrahydro- 1H-pyrrolizin- 7a(5H)- yl)methoxy) quinazolin-7- yl)-5,6- difluoronaphthalen- 2-ol | 594.2 (M + H)⁺ | ¹H NMR (400 MHz, METHANOL- d4) δ ppm 7.83 (d, J = 10.5 Hz, 1 H), 7.77 (d, J = 6.7 Hz, 1 H), 7.62 (ddd, J = 9.3, 4.8, 1.6 Hz, 1 H), 7.31 − 7.45 (m, 2 H), 7.13 (d, J = 2.3 Hz, 1 H), 5.49 − 5.75 (m, 1 H), 4.70 − 4.80 (m, 4 H), 4.28 (br s, 2 H), 3.86 − 4.06 (m, 5 H), 3.35 − 3.54 (m, 1 H), 2.56 - 2.86 (m, 2 H), 2.33 − 2.52 (m, 3 H), 2.14 − 2.29 (m, 5 H). |
| 83 | 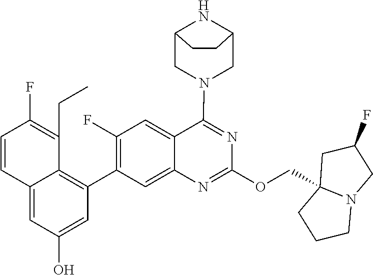 [SMILES: CCc1c(F)ccc2cc(O)cc(-c3cc4nc(OC[C@@]56CCCN5C[C@H](F)C6)nc(N5CC6CCC(C5)N6)c4cc3F)c12] | 4-(4-((1R,5S)- 3,8- diazabicyclo[3.2. 1]octan-3-yl)-6- fluoro-2- (((2R,7aS)-2- fluorotetrahydro- 1H-pyrrolizin- 7a(5H)-yl)methoxy) quinazolin-7-yl)-5- ethyl-6- fluoronaphthalen- 2-ol | 604.2 (M + H)⁺ | ¹H NMR (400 MHz, METHANOL- d4) δ ppm 7.76 − 7.83 (m, 2 H), 7.69 (dd, J = 9.1, 5.7 Hz, 1 H), 7.24 − 7.31 (m, 2 H), 6.95 (d, J = 2.5 Hz, 1 H), 5.48 − 5.72 (m, 1 H), 4.60 − 4.74 (m, 5 H), 4.29 (br s, 1 H), 3.79 − 4.01 (m, 5 H), 3.47 − 3.55 (m, 1 H), 2.55 − 2.82 (m, 3 H), 2.30 − 2.49 (m, 4 H), 2.16 − 2.29 (m, 5 H), 0.79 (td, J = 7.4, 1.6 Hz, 3 H). |
| 84 | 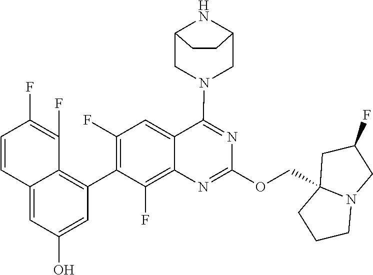 [SMILES: Oc1cc(-c2c(F)cc3c(N4CC5CCC(C4)N5)nc(OC[C@@]45CCCN4C[C@H](F)C5)nc3c2F)c2c(F)c(F)ccc2c1] | 4-(4-((1R,5S)- 3,8- diazabicyclo[3.2. 1]octan-3-yl)-6,8- difluoro-2- (((2R,7aS)-2- fluorotetrahydro- 1H-pyrrolizin- 7a(5H)-yl)methoxy) quinazolin-7- yl)-5,6- difluoronaphthalen- 2-ol | 612.2 (M + H)⁺ | ¹H NMR (400 MHz, METHANOL- d4) δ ppm 7.70 (dd, J = 9.93, 1.36 Hz, 1 H) 7.61 − 7.67 (m, 1 H) 7.43 (d, J = 7.73 Hz, 1 H) 7.36 (t, J = 2.19 Hz, 1 H) 7.17 (s, 1 H) 5.48 − 5.69 (m, 1 H) 4.71 (d, J = 6.27 Hz, 4 H) 4.24 − 4.33 (m, 2 H) 3.80 − 4.10 (m, 5 H) 3.43 − 3.55 (m, 1 H) 2.55 − 2.80 (m, 2 H) 2.30 − 2.51 (m, 3 H) 2.19 (br s, 5 H). |
| 85 | 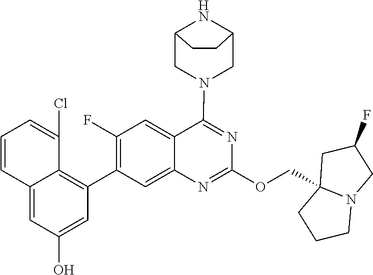 [SMILES: Oc1cc(-c2cc3nc(OC[C@@]45CCCN4C[C@H](F)C5)nc(N4CC5CCC(C4)N5)c3cc2F)c2c(Cl)cccc2c1] | 4-(4-((1R,5S)- 3,8- diazabicyclo[3.2. 1]octan-3-yl)-6- fluoro-2- (((2R,7aS)-2- fluorotetrahydro- 1H-pyrrolizin- 7a(5H)- yl)methoxy) quinazolin-7-yl)-5- chloronaphthalen- 2-ol | 592.2 (M + H)⁺ | ¹H NMR (400 MHz, METHANOL- d4) δ ppm 7.69 − 7.78 (m, 3 H), 7.33 − 7.40 (m, 3 H), 7.04 (d, J = 2.5 Hz, 1 H), 5.46 − 5.72 (m, 1 H), 4.58 − 4.75 (m, 4 H), 4.27 (br s, 2 H), 3.77 − 4.01 (m, 5 H), 3.44 − 3.55 (m, 1 H), 2.58 − 2.83 (m, 2 H), 2.33 − 2.50 (m, 3 H), 2.15 − 2.28 (m, 5 H). |
| 86 | 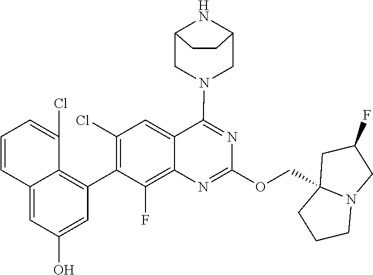 [SMILES: Oc1cc(-c2c(Cl)cc3c(N4CC5CCC(C4)N5)nc(OC[C@@]45CCCN4C[C@H](F)C5)nc3c2F)c2c(Cl)cccc2c1] | 4-(4-((1R,5S)- 3,8- diazabicyclo[3.2. 1]octan-3-yl)-6- chloro-8-fluoro- 2-(((2R,7aS)-2- fluorotetrahydro- 1H-pyrrolizin- 7a(5H)-yl)methoxy) quinazolin- 7-yl)-5- chloronaphthalen -2-ol | 626.2 (M + H)⁺ | ¹H NMR (400 MHz, METHANOL- d4) δ ppm 7.96 (d, J = 1.5 Hz, 1 H), 7.78 (dd, J = 8.2, 1.3 Hz, 1 H), 7.32 − 7.40 (m, 3 H), 7.00 (dd, J = 2.5, 0.8 Hz, 1 H), 5.46 − 5.71 (m, 1 H), 4.60 − 4.77 (m, 4 H), 4.27 (br d, J = 8.2 Hz, 2 H), 3.83 − 4.06 (m, 5 H), 3.43 − 3.53 (m, 1 H), 2.55 − 2.81 (m, 2 H), 2.32 − 2.48 (m, 3 H), 2.10 − 2.25 (m, 5 H). |
| 87 | 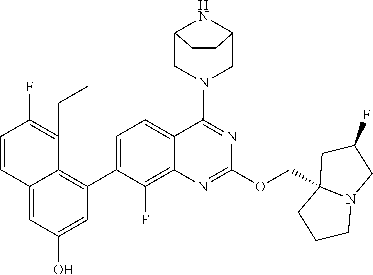 [SMILES: CCc1c(F)ccc2cc(O)cc(-c3ccc4c(N5CC6CCC(C5)N6)nc(OC[C@@]56CCCN5C[C@H](F)C6)nc4c3F)c12] | 4-(4-((1R,5S)- 3,8- diazabicyclo[3.2. 1]octan-3-yl)-8- fluoro-2- (((2R,7aS)-2- fluorotetrahydro- 1H-pyrrolizin- 7a(5H)- yl)methoxy) quinazolin-7-yl)-5- ethyl-6- fluoronaphthalen- 2-ol | 604.3 (M + H)⁺ | ¹H NMR (400 MHz, METHANOL- d4) δ ppm 8.34 − 8.48 (m, 1 H), 7.96 (t, J = 8.4 Hz, 1 H), 7.69 (dd, J = 9.0, 5.9 Hz, 1 H), 7.39 − 7.59 (m, 1 H), 7.22 − 7.35 (m, 1 H), 7.07 − 7.19 (m, 1 H), 6.94 − 7.01 (m, 1 H), 5.44 − 5.71 (m, 1 H), 4.72 − 4.84 (m, 4 H), 4.31 (br s, 2 H), 3.91 − 4.12 (m, 4 H), 3.44 − 3.64 (m, 1 H), 3.38 − 3.39 (m, 1 H), 2.70 − 2.84 (m, 1 H), 2.57 − 2.68 (m, 1 H), 2.24 − 2.53 (m, 6 H), 2.17 − 2.24 (m, 4 H), 0.70 − 0.88 (m, 3 H). |
| 88 | 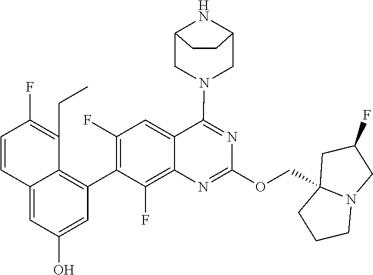 [SMILES: CCc1c(F)ccc2cc(O)cc(-c3c(F)cc4c(N5CC6CCC(C5)N6)nc(OC[C@@]56CCCN5C[C@H](F)C6)nc4c3F)c12] | 4-(4-((1R,5S)- 3,8- diazabicyclo[3.2. 1]octan-3-yl)-6,8- difluoro-2- (((2R,7aS)-2- fluorotetrahydro- 1H-pyrrolizin- 7a(5H)- yl)methoxy) quinazolin-7-yl)-5- ethyl-6- fluoronaphthalen- 2-ol | 622.2 (M + H)⁺ | ¹H NMR (400 MHz, METHANOL- d4) δ ppm 7.72 (s, 2 H) 7.34 (d, J = 2.70 Hz, 1 H) 7.24 − 7.32 (m, 1 H) 6.99 (d, J = 2.70 Hz, 1 H) 5.48 − 5.69 (m, 1 H) 4.62 − 4.80 (m, 4 H) 4.24 − 4.32 (m, 2 H) 3.79 − 4.09 (m, 5 H) 3.42 − 3.56 (m, 1 H) 2.51 − 2.80 (m, 3 H) 2.32 − 2.50 (m, 4 H) 2.12 − 2.27 (m, 5 H) 0.82 (br d, J = 1.87 Hz, 3 H). |
| 89 | 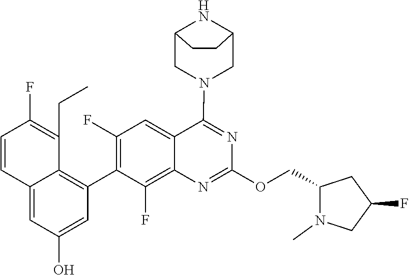 [SMILES: CCc1c(F)ccc2cc(O)cc(-c3c(F)cc4c(N5CC6CCC(C5)N6)nc(OC[C@@H]5C[C@@H](F)CN5C)nc4c3F)c12] | 4-(4-((1R,5S)- 3,8- diazabicyclo[3.2. 1]octan-3-yl)-6,8- difluoro-2- (((2S,4R)-4- fluoro-1- methylpyrrolidin- 2-yl)methoxy) quinazolin-7-yl)-5- ethyl-6- fluoronaphthalen- 2-ol | 596.0 (M + H)⁺ | ¹H NMR (400 MHz, METHANOL- d4) δ ppm 7.65 − 7.75 (m, 2 H) 7.31 − 7.35 (m, 1 H) 7.23 − 7.30 (m, 1 H) 6.96 − 7.02 (m, 1 H) 5.36 − 5.59 (m, 1 H) 4.92 − 5.01 (m, 1 H) 4.61 − 4.78 (m, 3 H) 4.26 (br s, 3 H) 3.95 − 4.18 (m, 1 H) 3.77 − 3.93 (m, 2 H) 3.57 − 3.76 (m, 1 H) 3.20 (s, 3 H) 2.35 − 2.78 (m, 4 H) 2.12 − 2.29 (m, 4 H) 0.82 (t, J = 7.36 Hz, 3 H). |
| 90 | 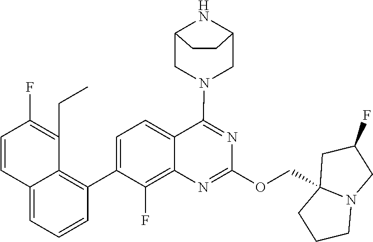 [SMILES: CCc1c(F)ccc2cccc(-c3ccc4c(N5CC6CCC(C5)N6)nc(OC[C@@]56CCCN5C[C@H](F)C6)nc4c3F)c12] | 4-((1R,5S)-3,8- diazabicyclo[3.2. 1]octan-3-yl)-7- (8-ethyl-7- fluoronaphthalen- 1-yl)-8-fluoro-2- (((2R,7aS)-2- fluorotetrahydro- 1H-pyrrolizin- 7a(5H)- yl)methoxy) quinazoline | 588.1 (M + H)⁺ | ¹H NMR (400 MHz, METHANOL- d4) δ ppm 8.07 − 8.12 (m, 1 H), 7.97 − 8.05 (m, 2 H), 7.51 − 7.64 (m, 2 H), 7.40 − 7.49 (m, 2 H), 5.54 − 5.74 (m, 1 H), 4.74 − 4.84 (m, 4 H), 4.29 − 4.40 (m, 2 H), 3.89 − 4.15 (m, 5 H), 3.49 − 3.62 (m, 1 H), 2.37 − 2.73 (m, 7 H), 2.21 − 2.30 (m, 5 H), 0.87 (s, 3 H). |
| 91 | 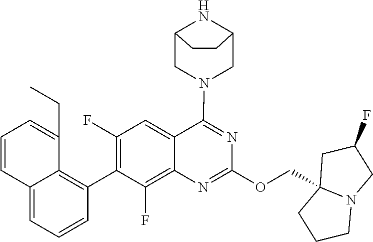 [SMILES: CCc1cccc2cccc(-c3c(F)cc4c(N5CC6CCC(C5)N6)nc(OC[C@@]56CCCN5C[C@H](F)C6)nc4c3F)c12] | 4-((1R,5S)-3,8- diazabicyclo[3.2. 1]octan-8-yl)-7- (8- ethylnaphthalen- 1-yl)-6,8- difluoro-2- (((2R,7aS)-2- fluorotetrahydro- 1H-pyrrolizin- 7a(5H)- yl)methoxy) quinazoline | 588.2 (M + H)⁺ | ¹H NMR (400 MHz, METHANOL- d4) δ ppm 8.04 − 8.11 (m, 1 H) 7.87 − 7.94 (m, 1 H) 7.79 (dd, J = 9.41, 1.67 Hz, 1 H) 7.58 (dd, J = 8.15, 7.11 Hz, 1 H) 7.48 − 7.54 (m, 1 H) 7.42 (d, J = 6.27 Hz, 1 H) 7.37 (d, J = 7.11 Hz, 1 H) 5.48 − 5.68 (m, 1 H) 5.15 − 5.27 (m, 2 H) 4.70 (s, 2 H) 3.81 − 4.08 (m, 3 H) 3.65 − 3.79 (m, 2 H) 3.49 (s, 3 H) 2.49 − 2.81 (m, 4 H) 2.27 − 2.48 (m, 5 H) 2.10 − 2.25 (m, 3 H) 0.95 (d, J = 1.25 Hz, 3 H). |
| 92 | 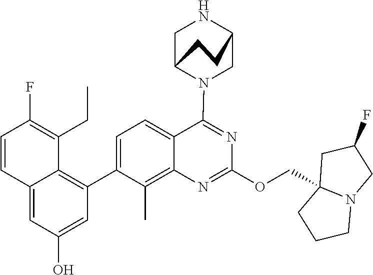 | 4-(4-((1S,4S)-2,5- diazabicyclo[2.2. 2]octan-2-yl)-2- (((2R,7aS)-2- fluorotetrahydro- 1H-pyrrolizin- 7a(5H)- yl)methoxy)-8- methylquinazolin- 7-yl)-5-ethyl-6- fluoronaphthalen- 2-ol | 600.3 (M + H)⁺ | ¹H NMR (400 MHz, METHANOL- d4) δ ppm 6.85 − 6.93 (m, 1 H), 6.52 − 6.60 (m, 1 H), 5.90 − 6.00 (m, 1 H), 5.53 − 5.61 (m, 1 H), 5.45 − 5.51 (m, 1 H), 4.02 − 4.20 (m, 1 H), 3.59 − 3.65 (m, 1 H), 3.45 − 3.50 (m, 1 H), 3.22 (s, 2 H), 2.78 − 3.08 (m, 2 H), 2.34 − 2.51 (m, 5 H), 1.96 − 2.12 (m, 2 H), 0.75 (br d, J = 7.5 Hz, 17 H), −0.87 − −0.79 (m, 3 H). |
| 93 | 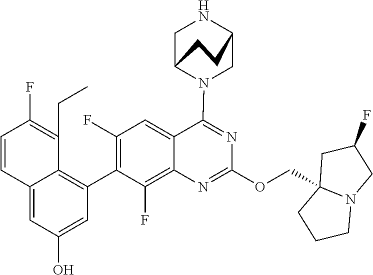 | 4-(4-((1S,4S)-2,5- diazabicyclo[2.2. 2]octan-2-yl)-6,8- difluoro-2- (((2R,7aS)-2- fluorotetrahydro- 1H-pyrrolizin- 7a(5H)- yl)methoxy) quinazolin-7-yl)-5- ethyl-6- fluoronaphthalen- 2-ol | 622.2 (M + H)⁺ | ¹H NMR (400 MHz, METHANOL- d4) δ ppm 7.87 (dd, J = 10.16, 1.45 Hz, 1 H) 7.71 (dd, J = 9.12, 6.01 Hz, 1 H) 7.33 (d, J = 2.70 Hz, 1 H) 7.28 (s, 1 H) 6.96 − 7.02 (m, 1 H) 5.48 − 5.68 (m, 1 H) 5.14 − 5.22 (m, 1 H) 4.69 (t, J = 2.59 Hz, 2 H) 4.50 − 4.58 (m, 1 H) 4.37 − 4.44 (m, 1 H) 3.85 − 4.10 (m, 5 H) 3.55 − 3.64 (m, 1 H) 3.41 − 3.52 (m, 1 H) 2.53 − 2.80 (m, 3 H) 2.29 − 2.52 (m, 5 H) 2.05 − 2.26 (m, 4 H) 0.83 (br d, J = 3.11 Hz, 3 H). |
| 94 | 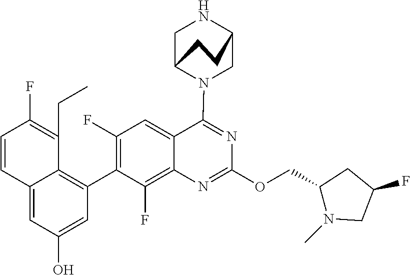 | 4-(4-((1S,4S)-2,5- diazabicyclo[2.2. 2]octan-2-yl)-6,8- difluoro-2- (((2S,4R)-4- fluoro-1- methylpyrrolidin- 2- yl)methoxy)quina zolin-7-yl)-5- ethyl-6- fluoronaphthalen- 2-ol | 596.3 (M + H)⁺ | ¹H NMR (400 MHz, METHANOL- d4) δ ppm 7.82 − 7.92 (m, 1 H) 7.71 (dd, J = 9.02, 5.91 Hz, 1 H) 7.33 (d, J = 2.70 Hz, 1 H) 7.28 (s, 1 H) 6.95 − 7.02 (m, 1 H) 5.37 − 5.63 (m, 1 H) 5.12 − 5.22 (m, 1 H) 4.94 − 5.02 (m, 1 H) 4.68 − 4.77 (m, 1 H) 4.48 − 4.58 (m, 1 H) 4.36 − 4.45 (m, 1 H) 4.24 − 4.34 (m, 1 H) 3.96 − 4.14 (m, 2 H) 3.82 − 3.93 (m, 1 H) 3.55 − 3.75 (m, 2 H) 3.19 (s, 3 H) 2.41 − 2.75 (m, 5 H) 2.22 − 2.32 (m, 1 H) 2.13 (br d, J = 2.28 Hz, 2 H) 0.83 (br d, J = 3.94 Hz, 3 H). |
| 95 | 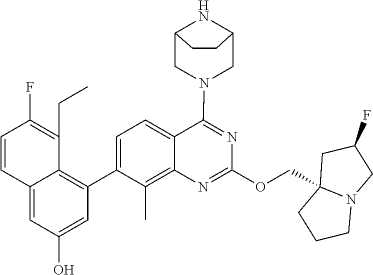 [SMILES: CCc1c(F)ccc2cc(O)cc(-c3ccc4c(N5CC6CCC(C5)N6)nc(OC[C@@]56CCCN5C[C@H](F)C6)nc4c3C)c12] | 4-(4-((1R,5S)- 3,8- diazabicyclo[3.2. 1]octan-3-yl)-2- (((2R,7aS)-2- fluorotetrahydro- 1H-pyrrolizin- 7a(5H)- yl)methoxy)-8- methylquinazolin- 7-yl)-5-ethyl-6- fluoronaphthalen- 2-ol | 600.1 (M + H)⁺ | ¹H NMR (400 MHz, METHANOL- d4) δ ppm 8.32 − 8.44 (m, 1 H), 7.87 − 8.00 (m, 1 H), 7.41 − 7.50 (m, 1 H), 7.20 − 7.30 (m, 1 H), 7.02 − 7.10 (m, 1 H), 6.75 − 6.86 (m, 1 H), 5.52 − 5.73 (m, 1 H), 4.96 − 5.02 (m, 1 H), 4.54 − 4.75 (m, 4 H), 4.22 − 4.33 (m, 2 H), 3.76 − 4.10 (m, 5 H), 3.45 − 3.57 (m, 1 H), 2.28 (s, 16 H), 0.61 − 0.76 (m, 3 H). |
| 96 | 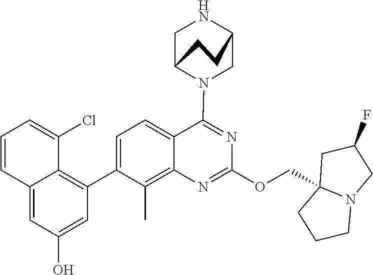 | 4-(4-((1S,4S)-2,5- diazabicyclo[2.2. 2]octan-2-yl)-2- (((2R,7aS)-2- fluorotetrahydro- 1H-pyrrolizin- 7a(5H)- yl)methoxy)-8- methylquinazolin- 7-yl)-5- chloronaphthalen- 2-ol | 588.1 (M + H)⁺ | ¹H NMR (400 MHz, METHANOL- d4) δ ppm 8.02 (d, J = 8.6 Hz, 1 H), 7.76 (dd, J = 8.2, 1.3 Hz, 1 H), 7.28 − 7.38 (m, 4 H), 6.91 (d, J = 2.5 Hz, 1 H), 0.00 (td, J = 51.6, 3.6 Hz, 1 H), 5.14 (br s, 1 H), 4.71 − 4.75 (m, 2 H), 4.47 − 4.56 (m, 1 H), 4.33 − 4.42 (m, 1 H), 3.84 − 4.10 (m, 5 H), 3.46 − 3.64 (m, 2 H), 2.57 − 2.85 (m, 2 H), 2.35 − 2.56 (m, 4 H), 2.28 (s, 7 H). |
| 97 | 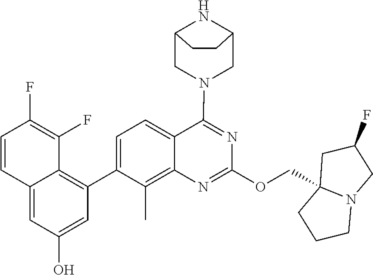 [SMILES: Cc1c(-c2cc(O)cc3ccc(F)c(F)c23)ccc2c(N3CC4CCC(C3)N4)nc(OC[C@@]34CCCN3C[C@H](F)C4)nc12] | 4-(4-((1R,5S)- 3,8- diazabicyclo[3.2. 1]octan-3-yl)-2- (((2R,7aS)-2- fluorotetrahydro- 1H-pyrrolizin- 7a(5H)- yl)methoxy)-8- methylquinazolin- 7-yl)-5,6- difluoronaphthalen- 2-ol | 590.2 (M + H)⁺ | ¹H NMR (400 MHz, METHANOL- d4) δ ppm 8.00 (d, J = 8.6 Hz, 1 H), 7.62 (dd, J = 8.7, 4.5 Hz, 1 H), 7.36 − 7.48 (m, 2 H), 7.26 − 7.33 (m, 1 H), 6.98 (d, J = 2.3 Hz, 1 H), 0.00 (dd, J = 51.8, 4.2 Hz, 1 H), 4.29 (br s, 2 H), 3.86 − 4.13 (m, 6 H), 3.47 − 3.57 (m, 1 H), 2.15 − 2.87 (m, 14 H). |
| 98 | 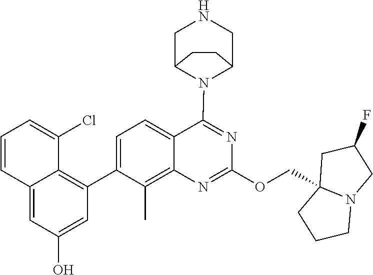 [SMILES: Cc1c(-c2cc(O)cc3cccc(Cl)c23)ccc2c(N3C4CCC3CNC4)nc(OC[C@@]34CCCN3C[C@H](F)C4)nc12] | 4-(4-((1R,5S)- 3,8- diazabicyclo[3.2. 1]octan-8-yl)-2- (((2R,7aS)-2- fluorotetrahydro- 1H-pyrrolizin- 7a(5H)- yl)methoxy)-8- methylquinazolin- 7-yl)-5- chloronaphthalen- 2-ol | 588.1 (M + H)⁺ | ¹H NMR (400 MHz, METHANOL- d4) δ ppm 8.00 − 8.04 (m, 1 H), 7.77 (d, J = 8.2 Hz, 1 H), 7.45 (d, J = 8.6 Hz, 1 H), 7.25 − 7.41 (m, 3 H), 6.92 (d, J = 2.5 Hz, 1 H), 5.62 (td, J = 51.8, 4.2 Hz, 1 H), 5.35 (br s, 2 H), 3.47 − 4.13 (m, 9 H), 2.46 − 2.86 (m, 3 H), 2.33 − 2.44 (m, 4 H), 2.29 (s, 6 H), 2.00 − 2.32 (m, 1 H). |

### 4-(4-((1R,5S)-3,8-Diazabicyclo[3.2.1]octan-8-yl)-8-fluoro-2-(((2R,7aS)-2-fluorotetrahydro-1H-pyrrolizin-7a(511)-yl)methoxy)quinazolin-7-yl)-5-ethylnaphthalen-2-ol (Example 99

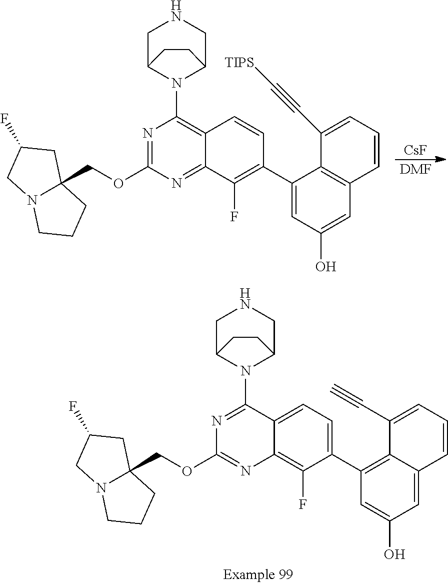

4-(4-((1R,5S)-3,8-Diazabicyclo[3.2.1]octan-8-yl)-8-fluoro-2-(((2R,7aS)-2-fluorotetrahydro-1H-pyrrolizin-7a(5H)-yl)methoxy)quinazolin-7-yl)-5-ethynylnaphthalen-2-ol. To an 8-mL vial was added 4-(4-((1R,5S)-3,8-diazabicyclo[3.2.1]octan-8-yl)-8-fluoro-2-(((2R,7aS)-2-fluorotetrahydro-1H-pyrrolizin-7a(5H)-yl)methoxy)quinazolin-7-yl)-5-((triisopropylsilyl)ethynyl)naphthalen-2-ol (17.0 mg, 0.023 mmol, synthesized according to Example 1), cesium fluoride (18 mg, 0.12 mmol, Sigma-Aldrich Corporation) and N,N-dimethylformamide (0.23 mL). The reaction was stirred at room temperature overnight. The crude product was purified by reverse phase HPLC to yield 4-(4-((1R,5S)-3,8-diazabicyclo[3.2.1]octan-8-yl)-8-fluoro-2-(((2R,7aS)-2-fluorotetrahydro-1H-pyrrolizin-7a(5H)-yl)methoxy)quinazolin-7-yl)-5-ethynylnaphthalen-2-ol as bis(2,2,2-trifluoroacetate) (9.8 mg, 0.017 mmol, 73% yield). m/z (ESI, +ve ion): 582.2 (M+H)⁺. ¹H NMR (400 MHz, METHANOL-d4) δ ppm 7.93-7.99 (m, 1H), 7.83 (s, 1H), 7.45-7.55 (m, 2H), 7.38-7.44 (m, 1H), 7.29-7.33 (m, 1H), 7.05-7.09 (m, 1H), 5.45-5.68 (m, 1H), 5.28 (br s, 2H), 4.63-4.80 (m, 2H), 3.81-4.09 (m, 3H), 3.62-3.77 (m, 2H), 3.41-3.54 (m, 3H), 2.89-2.94 (m, 1H), 2.55-2.76 (m, 2H), 2.27-2.52 (m, 5H), 2.06-2.25 (m, 3H).

### 4-((1R,5S)-3,8-Diazabicyclo[3.2.1]octan-8-yl)-7-(8-ethynylnaphthalen-1-yl)-6,8-difluoro-2-(((2R,7aS)-2-fluorotetrahydro-1H-pyrrolizin-7a(5H)-yl)methoxy)quinazoline Example 100

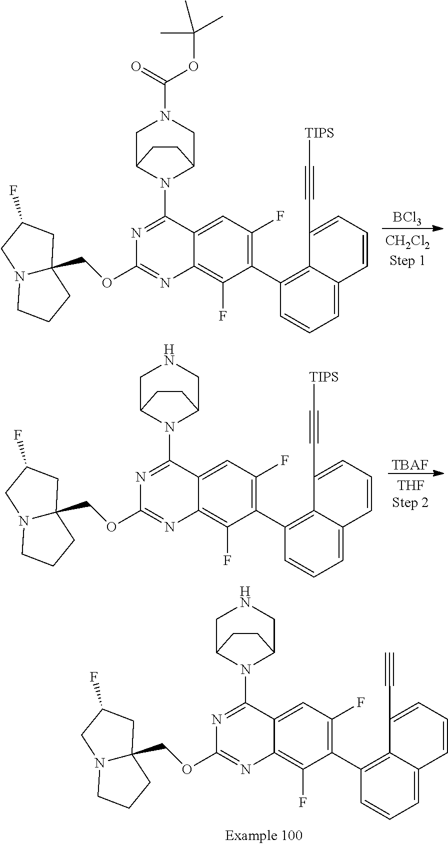

Step 1: 4-((1R,5S)-3,8-Diazabicyclo[3.2.1]octan-8-yl)-6,8-difluoro-2-(((2R,7aS)-2-fluorotetrahydro-1H-pyrrolizin-7a(5H)-yl)methoxy)-7-(8-((triisopropylsilyl)ethynyl)naphthalen-1-yl)quinazoline. A solution of tert-butyl (1R,5S)-8-(6,8-difluoro-2-(((2R,7aS)-2-fluorotetrahydro-1H-pyrrolizin-7a(5H)-yl)methoxy)-7-(8-((triisopropylsilyl)ethynyl)naphthalen-1-yl)quinazolin-4-yl)-3,8-diazabicyclo[3.2.1]octane-3-carboxylate (15.4 mg, 0.018 mmol, synthesized according to Example 1) in dichloromethane (0.61 mL) was cooled to 0° C. and then boron trichloride (65.4 μL, 0.065 mmol) was added dropwise. The reaction was stirred for 30 min at this temperature. The reaction was quenched by the addition of saturated sodium bicarbonate (3 mL). The mixture was then warmed to room temperature and transferred to a separatory funnel. The layers were separated and the aqueous layer was extracted with DCM (3×3 mL). The organic layers were then combined, dried with anhydrous sodium sulfate, filtered, and concentrated under reduced pressure to afford 4-((1R,5S)-3,8-diazabicyclo[3.2.1]octan-8-yl)-6,8-difluoro-2-(((2R,7aS)-2-fluorotetrahydro-1H-pyrrolizin-7a(5H)-yl)methoxy)-7-(8-((triisopropylsilyl)ethynyl)naphthalen-1-yl)quinazoline. m/z (ESI, +ve ion): 740.397 (M+H)⁺.

Step 2: 4-((1R,5S)-3,8-Diazabicyclo[3.2.1]octan-8-yl)-7-(8-ethynylnaphthalen-1-yl)-6,8-difluoro-2-(((2R,7aS)-2-fluorotetrahydro-1H-pyrrolizin-7a(5H)-yl)methoxy)quinazoline. An 8-mL vial was charged with 4-((1R,5S)-3,8-diazabicyclo[3.2.1]octan-8-yl)-6,8-difluoro-2-(((2R,7aS)-2-fluorotetrahydro-1H-pyrrolizin-7a(5H)-yl)methoxy)-7-(8-((triisopropylsilyl)ethynyl)naphthalen-1-yl)quinazoline was dissolved in THF (0.6 mL). The resultant solution was cooled to 0° C. and TBAF (1.0 M in THF, 33.0 μL, 0.033 mmol) was slowly added and the reaction was allowed to stir at this temperature. After 30 min, the reaction was concentrated under reduced pressure and purified via HPLC to provide 4-((1R,5S)-3,8-diazabicyclo[3.2.1]octan-8-yl)-7-(8-ethynylnaphthalen-1-yl)-6,8-difluoro-2-(((2R,7aS)-2-fluorotetrahydro-1H-pyrrolizin-7a(5H)-yl)methoxy)quinazoline as bis(2,2,2-trifluoroacetate) (4.3 mg, 7.37 μmol, 40% yield over two steps) as white solid. m/z (ESI, +ve ion): 584.3 (M+H)⁺. ¹H NMR (400 MHz, METHANOL-d₄) δ ppm 8.06-8.17 (m, 2H), 7.75-7.80 (m, 1H), 7.64-7.73 (m, 2H), 7.49-7.59 (m, 2H), 5.45-5.68 (m, 1H), 5.12-5.26 (m, 2H), 4.65-4.76 (m, 2H), 3.81-4.07 (m, 3H), 3.63-3.80 (m, 2H), 3.43-3.54 (m, 3H), 3.11-3.20 (m, 1H), 3.02-3.08 (m, 1H), 2.68 (s, 2H), 2.42-2.50 (m, 1H), 2.36 (br d, J=5.0 Hz, 3H), 2.08-2.24 (m, 3H).

### 4-((1R,5S)-3,8-Diazabicyclo[3.2.1]octan-8-yl)-7-(5-chloroisoquinolin-4-yl)-6,8-difluoro-2-(((2R,7aS)-2-fluorotetrahydro-1H-pyrrolizin-7a(5H)-yl)methoxy)quinazoline (Example 101

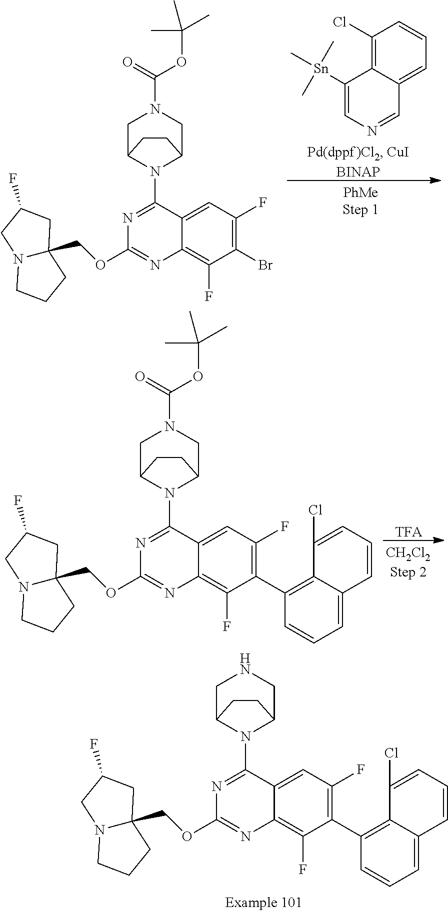

Step 1: tert-Butyl (1R,5S)-8-(7-(5-chloroisoquinolin-4-yl)-6,8-difluoro-2-(((2R,7aS)-2-fluorotetrahydro-1H-pyrrolizin-7a(5H)-yl)methoxy)quinazolin-4-yl)-3,8-diazabicyclo[3.2.1]octane-3-carboxylate. An 8-mL vial was charged with tert-butyl (1R,5S)-8-(7-bromo-6,8-difluoro-2-(((2R,7aS)-2-fluorotetrahydro-1H-pyrrolizin-7a(5H)-yl)methoxy)quinazolin-4-yl)-3,8-diazabicyclo[3.2.1]octane-3-carboxylate (45 mg, 0.073 mmol, synthesized according to Example 1), copper(I) iodide (4.2 mg, 0.022 mmol), 5-chloro-4-(trimethylstannyl)isoquinoline (48 mg, 0.15 mmol), and [1,1′-bis(diphenylphosphino)ferrocene]dichloropalladium(II) (5.4 mg, 7.35 μmol), rac-2,2-bis(diphenylphosphino)-1,1-binaphthyl (9.2 mg, 0.015 mmol), and toluene (0.74 mL). The reaction was stirred at 100° C. for 26 h. After cooling, the crude mixture was concentrated under reduced pressure and then purified by column chromatography on silica gel eluting with 0-50% 3:1 EtOAc/EtOH in heptane with 2% triethylamine additive to yield tert-butyl (1R,5S)-8-(7-(5-chloroisoquinolin-4-yl)-6,8-difluoro-2-(((2R,7aS)-2-fluorotetrahydro-1H-pyrrolizin-7a(5H)-yl)methoxy)quinazolin-4-yl)-3,8-diazabicyclo[3.2.1]octane-3-carboxylate. m/z (ESI, +ve ion): 695.2 (M+H)⁺.

Step 2: 4-((1R,5S)-3,8-Diazabicyclo[3.2.1]octan-8-yl)-7-(5-chloroisoquinolin-4-yl)-6,8-difluoro-2-(((2R,7aS)-2-fluorotetrahydro-1H-pyrrolizin-7a(5H)-yl)methoxy)quinazoline. tert-Butyl (1R,5S)-8-(7-(5-chloroisoquinolin-4-yl)-6,8-difluoro-2-(((2R,7aS)-2-fluorotetrahydro-1H-pyrrolizin-7a(5H)-yl)methoxy)quinazolin-4-yl)-3,8-diazabicyclo[3.2.1]octane-3-carboxylate was stirred in dichloromethane (2.5 mL) and trifluoroacetic acid (0.33 mL) at 35° C. until completion. Solvents were removed under reduced pressure. The crude product was purified by reverse phase HPLC to yield 4-((1R,5S)-3,8-diazabicyclo[3.2.1]octan-8-yl)-7-(5-chloroisoquinolin-4-yl)-6,8-difluoro-2-(((2R,7aS)-2-fluorotetrahydro-1H-pyrrolizin-7a(5H)-yl)methoxy)quinazoline as bis(2,2,2-trifluoroacetate) (18 mg, 0.031 mmol, 42% yield over two steps). m/z (ESI, +ve ion): 595.2 (M+H)⁺. ¹H NMR (400 MHz, METHANOL-d4) δ ppm 9.51 (s, 1H), 8.48 (s, 1H), 8.28-8.37 (m, 1H), 7.92-8.02 (m, 1H), 7.78 (s, 2H), 5.47-5.71 (m, 1H), 5.15-5.27 (m, 2H), 4.70 (br d, J=1.5 Hz, 2H), 3.84-4.08 (m, 3H), 3.63-3.80 (m, 2H), 3.48 (s, 3H), 2.55-2.79 (m, 2H), 2.27-2.52 (m, 5H), 2.17 (s, 3H).

### 4-((1R,5S)-3,8-Diazabicyclo[3.2.1]octan-8-yl)-6-fluoro-2-(((2R,7aS)-2-fluorotetrahydro-1H-pyrrolizin-7a(5H)-yl)methoxy)-7-(3-hydroxynaphthalen-1-yl)quinazolin-8-ol (Example 102

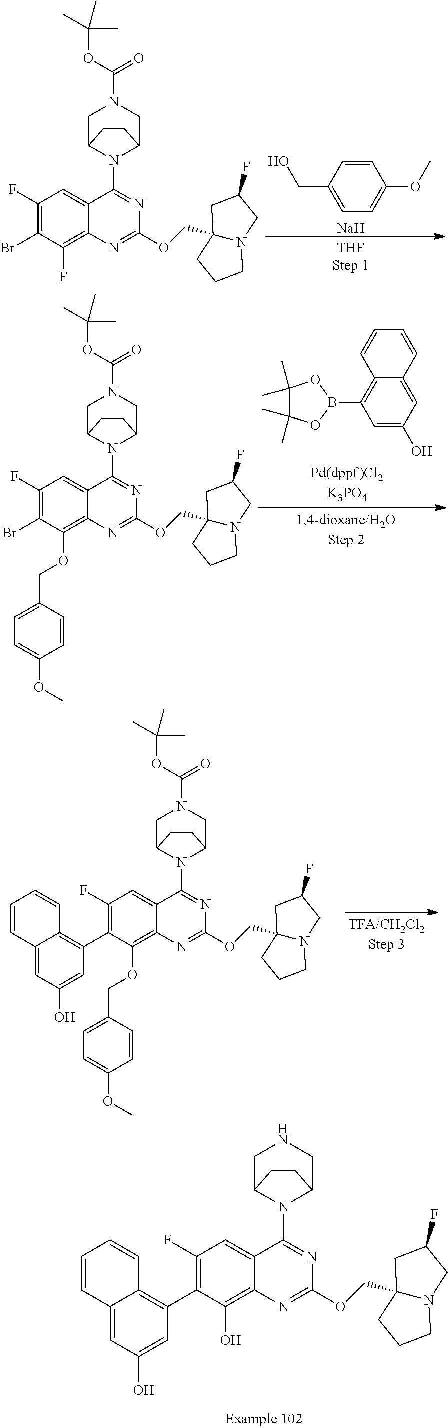

Step 1: tert-Butyl (1R,5S)-8-(7-bromo-6-fluoro-2-(((2R,7aS)-2-fluorotetrahydro-1H-pyrrolizin-7a(5H)-yl)methoxy)-8-((4-methoxybenzyl)oxy)quinazolin-4-yl)-3,8-diazabicyclo[3.2.1]octane-3-carboxylate. A solution of 4-methoxybenzyl alcohol (34 mg, 31 μL, 0.25 mmol) in tetrahydrofuran (0.82 mL) was cooled to 0° C. and then sodium hydride (9.8 mg, 0.25 mmol) was added. The reaction was allowed to stir at 0° C. After 5 min, the flask was warmed to room temperature for 25 min before re-cooling the flask to 0° C. In a separate flask, tert-butyl (1R,5S)-8-(7-bromo-6,8-difluoro-2-(((2R,7aS)-2-fluorotetrahydro-1H-pyrrolizin-7a(5H)-yl)methoxy)quinazolin-4-yl)-3,8-diazabicyclo[3.2.1]octane-3-carboxylate (75 mg, 0.12 mmol) was added and dissolved in tetrahydrofuran (1.6 mL). The solution was cooled to 0° C. and added dropwise to the flask containing 4-methoxybenzyl alcohol. The reaction was slowly warmed to room temperature over 2 h and then stirred for an additional 2 h at this temperature. The reaction was then quenched by the addition of water (1.5 mL) and saturated ammonium chloride (1.5 mL), and the aqueous layer was extracted with EtOAc (3×5 mL). The organic layers were combined, dried over anhydrous sodium sulfate, filtered, and concentrated under reduced pressure. The crude reaction mixture was then purified by column chromatography on silica gel eluting with a gradient of 0-50% of a 3:1 EtOAc:EtOH mixture in heptane with 2% triethylamine to provide tert-butyl (1R,5S)-8-(7-bromo-6-fluoro-2-(((2R,7aS)-2-fluorotetrahydro-1H-pyrrolizin-7a(5H)-yl)methoxy)-8-((4-methoxybenzyl)oxy)quinazolin-4-yl)-3,8-diazabicyclo[3.2.1]octane-3-carboxylate (90 mg, 0.12 mmol, 100% yield). m/z (ESI, +ve ion): 730.0 (M+H)⁺.

Step 2&3: 4-((1R,5S)-3,8-Diazabicyclo[3.2.1]octan-8-yl)-6-fluoro-2-(((2R,7aS)-2-fluorotetrahydro-1H-pyrrolizin-7a(5H)-yl)methoxy)-7-(3-hydroxynaphthalen-1-yl)quinazolin-8-ol. Prepared in an analogous manner to Example 1, as bis(2,2,2-trifluoroacetate). m/z (ESI, +ve ion): 574.2 (M+H)⁺. ¹H NMR (400 MHz, METHANOL-d4) δ ppm 7.74-7.80 (m, 1H), 7.40-7.47 (m, 2H), 7.33-7.37 (m, 1H), 7.26-7.31 (m, 1H), 7.16-7.23 (m, 1H), 7.09-7.13 (m, 1H), 5.46-5.65 (m, 1H), 5.13-5.25 (m, 2H), 4.73 (s, 2H), 3.75-4.02 (m, 3H), 3.65-3.74 (m, 2H), 3.41-3.52 (m, 3H), 2.55-2.75 (m, 2H), 2.30-2.49 (m, 5H), 2.08-2.21 (m, 3H).

### 4-((1R,5S)-3,8-diazabicyclo[3.2.1]octan-8-yl)-6-fluoro-2-(((2S,4R)-4-fluoro-1-methylpyrrolidin-2-yl)methoxy)-7-(3-hydroxynaphthalen-1-yl)quinazolin-8-ol (Example 103

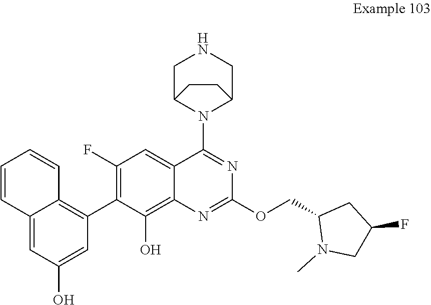 [SMILES: CN1C[C@H](F)C[C@H]1COc1nc(N2C3CCC2CNC3)c2cc(F)c(-c3cc(O)cc4ccccc34)c(O)c2n1]

Prepared in an analogous manner to Example 102, as bis(2,2,2-trifluoroacetate). m/z (ESI, +ve ion): 548.2 (M+H). ¹H NMR (400 MHz, METHANOL-d4) δ ppm 7.77 (s, 1H), 7.44 (d, J=9.8 Hz, 2H), 7.33-7.37 (m, 1H), 7.29 (d, J=2.5 Hz, 1H), 7.17-7.25 (m, 1H), 7.11 (s, 1H), 5.35-5.57 (m, 1H), 5.13-5.24 (m, 2H), 5.00-5.10 (m, 1H), 4.70-4.79 (m, 1H), 4.18-4.29 (m, 1H), 3.91-4.14 (m, 1H), 3.65-3.74 (m, 2H), 3.49-3.65 (m, 1H), 3.41-3.49 (m, 2H), 3.16 (d, J=2.9 Hz, 3H), 2.60-2.76 (m, 1H), 2.27-2.47 (m, 3H), 2.15 (d, J=8.2 Hz, 2H).

### 4-((1R,5S)-3,8-diazabicyclo[3.2.1]octan-8-yl)-7-(7-fluoro-8-vinylnaphthalen-1-yl)-2-(((2R,7aS)-2-fluorotetrahydro-1H-pyrrolizin-7a(5H)-yl)methoxy)quinazoline (Example 104

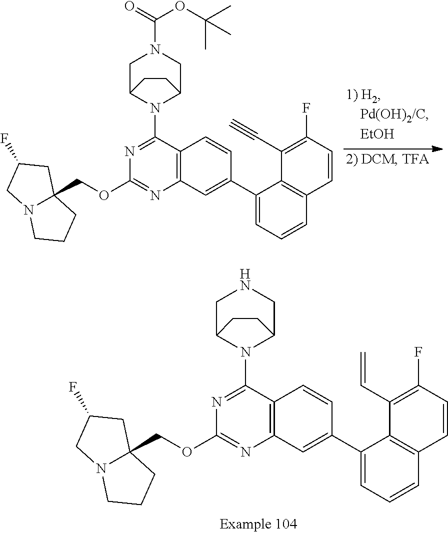 [SMILES: C#Cc1c(F)ccc2cccc(-c3ccc4c(N5C6CCC5CN(C(=O)OC(C)(C)C)C6)nc(OC[C@@]56CCCN5C[C@H](F)C6)nc4c3)c12 ; C=Cc1c(F)ccc2cccc(-c3ccc4c(N5C6CCC5CNC6)nc(OC[C@@]56CCCN5C[C@H](F)C6)nc4c3)c12]

A 25 mL vial was charged with Pd(OH)₂/C (9.49 mg, 0.068 mmol), tert-butyl (1R,5S)-8-(7-(8-ethynyl-7-fluoronaphthalen-1-yl)-2-(((2R,7aS)-2-fluorotetrahydro-1H-pyrrolizin-7a(5H)-yl)methoxy)quinazolin-4-yl)-3,8-diazabicyclo[3.2.1]octane-3-carboxylate (45 mg, 0.068 mmol) and EtOH (1 mL). The reaction was placed under a 45 psi H₂ atmosphere and was allowed to stir 18 h at room temperature. Upon completion, the mixture was filtered through a plug of celite and concentrated to dryness. The resulting orange oil was suspended in DCM (1 mL) and TFA (521 μL, 6.76 mmol) was added. The reaction was stirred for 1 h. Upon completion, the mixture was concentrated, resuspended in DMSO, filtered, and purified by reverse phase chromatography to give 4-((1R,5S)-3,8-diazabicyclo[3.2.1]octan-8-yl)-7-(7-fluoro-8-vinylnaphthalen-1-yl)-2-(((2R,7aS)-2-fluorotetrahydro-1H-pyrrolizin-7a(5H)-yl)methoxy)quinazoline (7.8 mg, 0.014 mmol, 20% yield) as a bis(2,2,2-trifluoroacetate) and yellow solid. m/z (ESI, +ve ion): 568.2 (M+H)⁺. ¹H NMR (400 MHz, METHANOL-d₄) δ ppm 8.14 (d, J=8.8 Hz, 1H), 7.73-7.82 (m, 2H), 7.33-7.49 (m, 4H), 7.17 (d, J=6.9 Hz, 1H), 5.36-5.76 (m, 4H), 5.21 (br dd, J=8.7, 3.7 Hz, 1H), 4.10 (br dd, J=17.5, 8.5 Hz, 1H), 3.83-4.01 (m, 4H), 3.63-3.79 (m, 3H), 3.42-3.60 (m, 5H), 2.16-2.40 (m, 9H).

### 4-(4-((1R,5S)-3,8-Diazabicyclo[3.2.1]octan-3-yl)-8-fluoro-2-(((2R,7aS)-2-fluorotetrahydro-1H-pyrrolizin-7a(5H)-yl)methoxy)-6-vinylquinazolin-7-yl)naphthalen-2-ol (Example 105

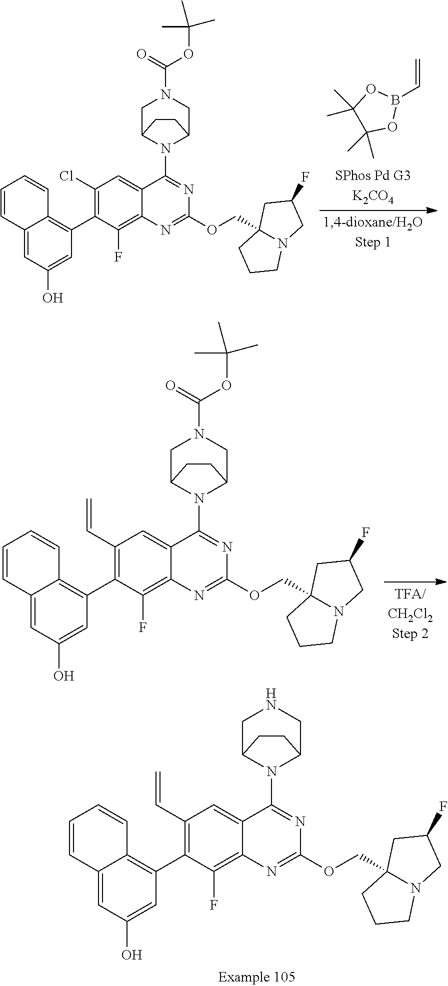 [SMILES: CC(C)(C)OC(=O)N1CC2CCC(C1)N2c1nc(OC[C@@]23CCCN2C[C@H](F)C3)nc2c(F)c(-c3cc(O)cc4ccccc34)c(Cl)cc12 ; C=CB1OC(C)(C)C(C)(C)O1 ; C=Cc1cc2c(N3C4CCC3CN(C(=O)OC(C)(C)C)C4)nc(OC[C@@]34CCCN3C[C@H](F)C4)nc2c(F)c1-c1cc(O)cc2ccccc12 ; C=Cc1cc2c(N3C4CCC3CNC4)nc(OC[C@@]34CCCN3C[C@H](F)C4)nc2c(F)c1-c1cc(O)cc2ccccc12]

Step 1: tert-Butyl (1R,5S)-3-(8-fluoro-2-(((2R,7aS)-2-fluorotetrahydro-1H-pyrrolizin-7a(5H)-yl)methoxy)-7-(3-hydroxynaphthalen-1-yl)-6-vinylquinazolin-4-yl)-3,8-diazabicyclo[3.2.1]octane-8-carboxylate. To a microwave vial was added tert-butyl (1R,5S)-3-(6-chloro-8-fluoro-2-(((2R,7aS)-2-fluorotetrahydro-1H-pyrrolizin-7a(5H)-yl)methoxy)-7-(3-hydroxynaphthalen-1-yl)quinazolin-4-yl)-3,8-diazabicyclo[3.2.1]octane-8-carboxylate (160 mg, 0.231 mmol, synthesize in a manner similar to Example 1), vinylboronic acid pinacol ester (182 mg, 0.200 mL, 1.179 mmol), (2-dicyclohexylphosphino-2′,6′-dimethoxybiphenyl) [2-(2′-amino-1,1′-biphenyl)]palladium(II) methanesulfonate (72.1 mg, 0.092 mmol), and potassium phosphate (255 mg, 1.202 mmol). The vial was purged with nitrogen and suspended in 1,4-dioxane (3 mL)/water (0.75 mL). The reaction was then heated in microwave to 150° C. for 1 h. The reaction mixture was concentrated and purified by ISCO Combiflash RF (4 g Redisep Gold column, using a gradient of 0-50% [3:1 EtOAc: EtOH, with 2% Et₃N]/Heptane) to afford tert-butyl (1R,5S)-3-(8-fluoro-2-(((2R,7aS)-2-fluorotetrahydro-1H-pyrrolizin-7a(5H)-yl)methoxy)-7-(3-hydroxynaphthalen-1-yl)-6-vinylquinazolin-4-yl)-3,8-diazabicyclo[3.2.1]octane-8-carboxylate (81 mg, 0.118 mmol, 51.2% yield) as white solid. m/z (ESI, +ve ion): 684.2 (M+H)⁺.

Step 2: 4-(4-((1R,5S)-3,8-Diazabicyclo[3.2.1]octan-3-yl)-8-fluoro-2-(((2R,7aS)-2-fluorotetrahydro-1H-pyrrolizin-7a(5H)-yl)methoxy)-6-vinylquinazolin-7-yl)naphthalen-2-ol. tert-Butyl (1R,5S)-3-(8-fluoro-2-(((2R,7aS)-2-fluorotetrahydro-1H-pyrrolizin-7a(5H)-yl)methoxy)-7-(3-hydroxynaphthalen-1-yl)-6-vinylquinazolin-4-yl)-3,8-diazabicyclo[3.2.1]octane-8-carboxylate (16.0 mg, 0.023 mmol) was dissolved in dichloromethane (0.5 mL). Trifluoroacetic acid (765 mg, 0.5 mL, 6.71 mmol) was added and the reaction mixture was stirred at rt for 1 h. The reaction mixture was concentrated and purified by HPLC to afford 4-(4-((1R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)-6-vinylquinazolin-7-yl)naphthalen-2-ol (13.0 mg, 0.022 mmol, 95% yield) as a bis(2,2,2-trifluoroacetate) and light-yellow solid. ¹H NMR (400 MHz, METHANOL-d4) δ ppm 8.11 (s, 1H), 7.78 (d, J=8.6 Hz, 1H), 7.43 (ddd, J=8.3, 5.1, 2.9 Hz, 1H), 7.29 (d, J=2.3 Hz, 1H), 7.19 (s, 1H), 7.18 (d, J-=4.4 Hz, 2H), 7.01 (s, 1H), 6.31 (dd, J=17.3, 11.1 Hz, 1H), 5.75-5.83 (m, 1H), 5.45-5.71 (m, 1H), 5.17 (d, J=11.3 Hz, 1H), 4.84-4.89 (m, 1H), 4.67-4.80 (m, 2H), 4.29 (br s, 2H), 3.82-4.07 (m, 5H), 3.48 (dt, J=10.1, 5.3 Hz, 1H), 2.55-2.86 (m, 2H), 2.29-2.50 (m, 3H), 2.12-2.24 (m, 5H). m/z (ESI, +ve ion): 584.2 (M+H)⁺.

### 4-(4-((1R,5S)-3,8-diazabicyclo[3.2.1]octan-3-yl)-6-ethyl-8-fluoro-2-(((2R,7aS)-2-fluorotetrahydro-1H-pyrrolizin-7a(5H)-yl)methoxy)quinazolin-7-yl)naphthalen-2-ol Example 106

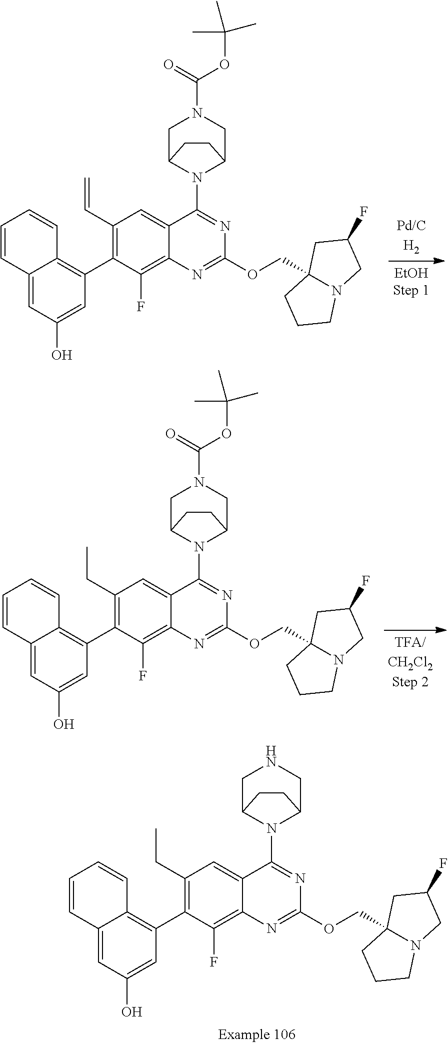 [SMILES: C=Cc1cc2c(N3C4CCC3CN(C(=O)OC(C)(C)C)C4)nc(OC[C@@]34CCCN3C[C@H](F)C4)nc2c(F)c1-c1cc(O)cc2ccccc12 ; CCc1cc2c(N3C4CCC3CN(C(=O)OC(C)(C)C)C4)nc(OC[C@@]34CCCN3C[C@H](F)C4)nc2c(F)c1-c1cc(O)cc2ccccc12 ; CCc1cc2c(N3C4CCC3CNC4)nc(OC[C@@]34CCCN3C[C@H](F)C4)nc2c(F)c1-c1cc(O)cc2ccccc12]

Step 1: tert-Butyl (1R,5S)-3-(6-ethyl-8-fluoro-2-(((2R,7aS)-2-fluorotetrahydro-1H-pyrrolizin-7a(5H)-yl)methoxy)-7-(3-hydroxynaphthalen-1-yl)quinazolin-4-yl)-3,8-diazabicyclo[3.2.1]octane-8-carboxylate. A test tube was charged with tert-butyl (1R,5S)-3-(8-fluoro-2-(((2R,7aS)-2-fluorotetrahydro-1H-pyrrolizin-7a(5H)-yl)methoxy)-7-(3-hydroxynaphthalen-1-yl)-6-vinylquinazolin-4-yl)-3,8-diazabicyclo[3.2.1]octane-8-carboxylate (65 mg, 0.095 mmol), and the solid was dissolved in ethanol (2.5 mL). To this 5% Pd(s)/carbon (20.23 mg, 9.51 μmol) was added and the reaction was placed under 40 Psi hydrogen gas and stirred at rt for 1 day. The reaction mixture was filtered through celite and washed with MeOH. The filtrate was concentrated and purified by HPLC to afford tert-butyl (1R,5S)-3-(6-ethyl-8-fluoro-2-(((2R,7aS)-2-fluorotetrahydro-1H-pyrrolizin-7a(5H)-yl)methoxy)-7-(3-hydroxynaphthalen-1-yl)quinazolin-4-yl)-3,8-diazabicyclo[3.2.1]octane-8-carboxylate as light yellow solid. m/z (ESI, +ve ion): 686.2 (M+H)⁺.

Step 2: 4-(4-((1R,5S)-3,8-Diazabicyclo[3.2.1]octan-3-yl)-6-ethyl-8-fluoro-2-(((2R,7aS)-2-fluorotetrahydro-1H-pyrrolizin-7a(5H)-yl)methoxy)quinazolin-7-yl)naphthalen-2-ol. tert-Butyl (1R,5S)-3-(6-ethyl-8-fluoro-2-(((2R,7aS)-2-fluorotetrahydro-1H-pyrrolizin-7a(5H)-yl)methoxy)-7-(3-hydroxynaphthalen-1-yl)quinazolin-4-yl)-3,8-diazabicyclo[3.2.1]octane-8-carboxylate was dissolved in dichloromethane (1 mL). Trifluoroacetic acid (765 mg, 0.5 mL, 6.71 mmol) was added and the reaction mixture was stirred at rt for 1 h. The reaction mixture was concentrated and purified by HPLC to afford 4-(4-((1R,5S)-3,8-diazabicyclo[3.2.1]octan-3-yl)-6-ethyl-8-fluoro-2-(((2R,7aS)-2-fluorotetrahydro-1H-pyrrolizin-7a(5H)-yl)methoxy)quinazolin-7-yl)naphthalen-2-ol (39.8 mg, 0.068 mmol, 71.5% yield) as a bis(2,2,2-trifluoroacetate) and light-yellow solid. ¹H NMR (400 MHz, METHANOL-d4) δ ppm 7.81 (s, 1H), 7.78 (d, J=8.4 Hz, 1H), 7.43 (dt, J=8.3, 4.0 Hz, 1H), 7.29 (d, J=2.3 Hz, 1H), 7.18 (d, J=4.4 Hz, 2H), 7.01-7.05 (m, 1H), 5.44-5.68 (m, 1H), 4.68-4.81 (m, 4H), 4.29 (br s, 2H), 3.81-4.07 (m, 5H), 3.47 (td, J=10.4, 6.0 Hz, 1H), 2.58-2.80 (m, 2H), 2.32-2.57 (m, 5H), 2.19 (s, 5H), 1.05 (t, J=7.5 Hz, 3H). m/z (ESI, +ve ion): 586.2 (M+H)⁺.

### 4-(4-((1R,5S)-3,8-Diazabicyclo[3.2.1]octan-3-yl)-8-fluoro-2-(((2R,7aS)-2-fluorotetrahydro-1H-pyrrolizin-7a(5H)-yl)methoxy)-6-methylquinazolin-7-yl)naphthalen-2-ol (Example 107

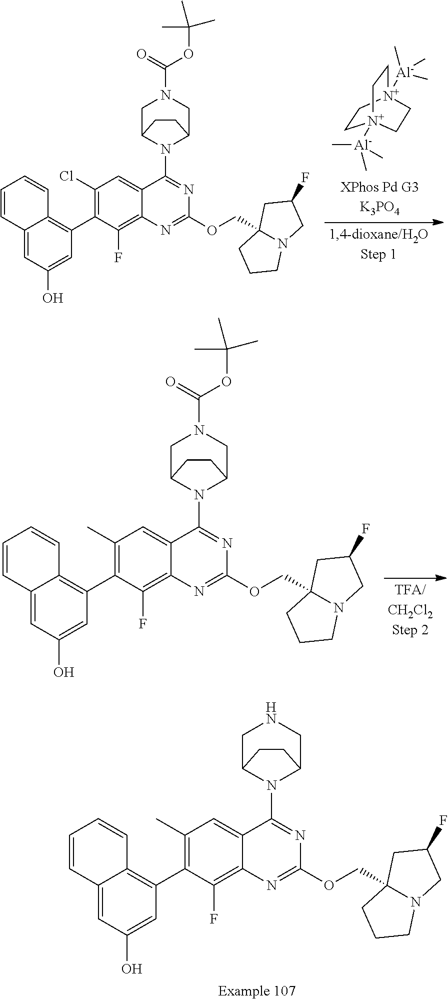

Step 1: tert-Butyl (1R,5S)-3-(8-fluoro-2-(((2R,7aS)-2-fluorotetrahydro-1H-pyrrolizin-7a(5H)-yl)methoxy)-7-(3-hydroxynaphthalen-1-yl)-6-methylquinazolin-4-yl)-3,8-diazabicyclo[3.2.1]octane-8-carboxylate. In a microwave vial, the mixture of tert-butyl (1R,5S)-3-(6-chloro-8-fluoro-2-(((2R,7aS)-2-fluorotetrahydro-1H-pyrrolizin-7a(5H)-yl)methoxy)-7-(3-hydroxynaphthalen-1-yl)quinazolin-4-yl)-3,8-diazabicyclo[3.2.1]octane-8-carboxylate (46 mg, 0.066 mmol), 1,4-diazabicyclo[2.2.2]octane bis(trimethylalumane) (34.1 mg, 0.133 mmol), and (2-dicyclohexylphosphino-2′,4′,6′-triisopropyl-1,1′-biphenyl)[2-(2′-amino-1,1′-biphenyl)]palladium(II) methanesulfonate (11.25 mg, 0.013 mmol) was suspended in degassed tetrahydrofuran (2 mL) and stirred at 70° C. for 1 h. The reaction mixture was concentrated and purified by ISCO Combiflash RF (4 g Redisep Gold column, using a gradient of 0-50% [3:1 EtOAc:EtOH, with 2% Et₃N]/Heptane) to afford tert-butyl (1R,5S)-3-(8-fluoro-2-(((2R,7aS)-2-fluorotetrahydro-1H-pyrrolizin-7a(5H)-yl)methoxy)-7-(3-hydroxynaphthalen-1-yl)-6-methylquinazolin-4-yl)-3,8-diazabicyclo[3.2.1]octane-8-carboxylate as white solid. m/z (ESI, +ve ion): 672.2 (M+H)⁺.

Step 2: 4-(4-((1R,5S)-3,8-Diazabicyclo[3.2.1]octan-3-yl)-8-fluoro-2-(((2R,7aS)-2-fluorotetrahydro-1H-pyrrolizin-7a(5H)-yl)methoxy)-6-methylquinazolin-7-yl)naphthalen-2-ol. tert-Butyl (1R,5S)-3-(8-fluoro-2-(((2R,7aS)-2-fluorotetrahydro-1H-pyrrolizin-7a(5H)-yl)methoxy)-7-(3-hydroxynaphthalen-1-yl)-6-methylquinazolin-4-yl)-3,8-diazabicyclo[3.2.1]octane-8-carboxylate was dissolved in 0.5 mL DCM. Trifluoroacetic acid (765 mg, 0.5 mL, 6.71 mmol) was added and the reaction mixture was stirred at rt for 1 h. The reaction mixture was concentrated and purified by HPLC to afford 4-(4-((1R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)-6-methylquinazolin-7-yl)naphthalen-2-ol (23.9 mg, 0.042 mmol, 62.9% yield) as a bis(2,2,2-trifluoroacetate) and white solid. ¹H NMR (400 MHz, METHANOL-d4) δ ppm 7.77-7.83 (m, 2H), 7.44 (ddd, J=8.3, 5.5, 2.5 Hz, 1H), 7.28 (d, J=2.5 Hz, 1H), 7.19 (d, J=4.9 Hz, 2H), 7.19 (s, 1H), 7.01 (t, J=2.2 Hz, 1H), 5.42-5.72 (m, 1H), 4.69-4.79 (m, 3H), 4.28 (br s, 2H), 3.82-4.07 (m, 5H), 3.43-3.51 (m, 1H), 2.53-2.82 (m, 2H), 2.31-2.48 (m, 3H), 2.12-2.23 (m, 8H). m/z (ESI, +ve ion): 571.9 (M+H)⁺.

### 4-(4-((8-Azabicyclo[3.2.1]oct-2-en-3-yl)-2-(((2R,7aS)-2-fluorotetrahydro-1H-pyrrolizin-7a(5H)-yl)methoxy)quinazolin-7-yl)naphthalen-2-ol (Example 108

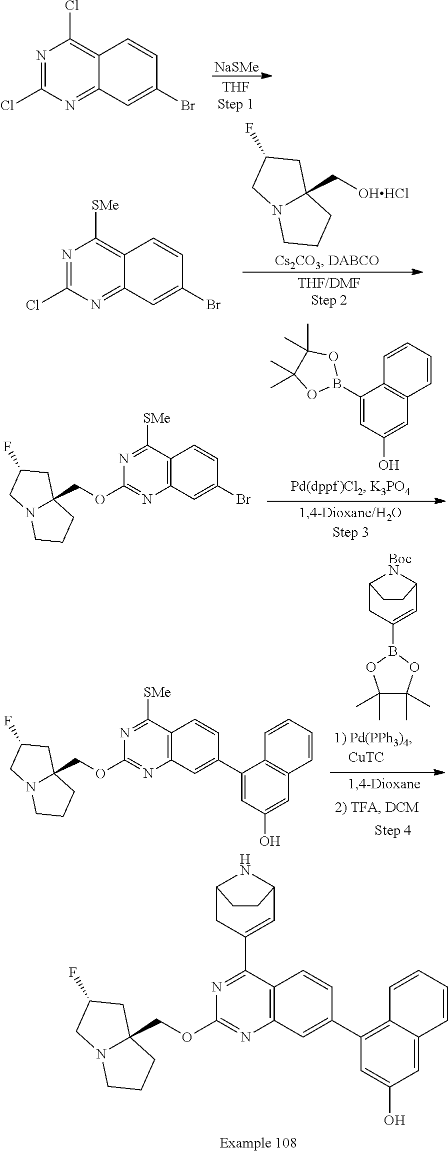

Step 1: 7-Bromo-2-chloro-4-(methylthio)quinazoline. To a stirring solution of 7-bromo-2,4-dichloroquinazoline (1.0 g, 3.60 mmol, pharmablock) in THF (40 mL) and water (1 mL) at 0° C. was added NaSMe (0.277 g, 3.96 mmol). The reaction was stirred at room temperature for 6 h. Upon completion, the mixture was dried with MgSO₄ and concentrated to give 7-bromo-2-chloro-4-(methylthio)quinazoline (1.04 g, 3.59 mmol, 100% yield) as a yellow solid, which was used in the following step without further manipulation. m/z (ESI, +ve ion): 288.9 (M+H)⁺.

Step 2: 7-Bromo-2-(((2R,7aS)-2-fluorotetrahydro-1H-pyrrolizin-7a(5H)-yl)methoxy)-4-(methylthio)quinazoline. The mixture of 7-bromo-2-chloro-4-(methylthio)quinazoline (1.05 g, 3.63 mmol), ((2R,7aS)-2-fluorotetrahydro-1H-pyrrolizin-7a(5H)-yl)methanol hydrochloride (1.064 g, 5.44 mmol, PharmaBlock), 1,4-diazabicyclo[2.2.2]octane (0.203 g, 1.813 mmol), and cesium carbonate (3.54 g, 10.88 mmol) was stirred in DMF (5 mL) and THF (30 mL) at 45° C. for 12 h. Upon completion, the reaction mixture was diluted with water and extracted with DCM. The organic layer was concentrated and purified by column chromatography eluting with a gradient of 0-100%(3:1 EtOAc:EtOH with 2% TEA) in heptane to afford 7-bromo-2-(((2R,7aS)-2-fluorotetrahydro-1H-pyrrolizin-7a(5H)-yl)methoxy)-4-(methylthio)quinazoline (968 mg, 2.348 mmol, 65% yield) as a yellow solid. m/z (ESI, +ve ion): 412.0 (M+H)⁺.

Step 3: 4-(2-(((2R,7aS)-2-Fluorotetrahydro-1H-pyrrolizin-7a(5H)-yl)methoxy)-4-(methylthio)quinazolin-7-yl)naphthalen-2-ol. A 100 mL round bottom flask was charged with 4-(4,4,5,5-tetramethyl-1,3,2-dioxaborolan-2-yl)naphthalen-2-ol (634 mg, 2.348 mmol, Aurum), potassium phosphate tribasic (1096 mg, 5.16 mmol), [1,1′-bis(diphenylphosphino)ferrocene]dichloropalladium(II) (344 mg, 0.470 mmol, Sigma-Aldrich Corporation) and 7-bromo-2-(((2R,7aS)-2-fluorotetrahydro-1H-pyrrolizin-7a(5H)-yl)methoxy)-4-(methylthio)quinazoline (968 mg, 2.348 mmol). The round bottom flask was purged with nitrogen gas and then the solids were suspended in degassed water (3.80 mL) and 1,4-dioxane (19 mL). The reaction was then stirred at 90° C. After 3 h, the reaction was cooled to room temperature and diluted with water (3 mL) and EtOAc (3 mL). The aqueous layer was extracted with EtOAc (3×30 mL). The organic layers were then combined, dried with sodium sulfate, filtered, and concentrated under reduced pressure. The crude residue was then purified by column chromatography on silica gel eluting with a gradient of 0-50% (3:1 EtOAc:EtOH with 2% TEA) in heptane to provide 4-(2-(((2R,7aS)-2-fluorotetrahydro-1H-pyrrolizin-7a(5H)-yl)methoxy)-4-(methylthio)quinazolin-7-yl)naphthalen-2-ol (354 mg, 0.744 mmol, 32% yield) as a brown solid. m/z (ESI, +ve ion): 476.1 (M+H)⁺.

Step 4: 4-(4-(8-azabicyclo[3.2.1]oct-2-en-3-yl)-2-(((2R,7aS)-2-fluorotetrahydro-1H-pyrrolizin-7a(5H)-yl)methoxy)quinazolin-7-yl)naphthalen-2-ol. A vial was charged with copper(I) thiophene-2-carboxylate (88 mg, 0.463 mmol), Pd(PPh₃)₄(48.6 mg, 0.042 mmol), tert-butyl 3-(4,4,5,5-tetramethyl-1,3,2-dioxaborolan-2-yl)-8-azabicyclo[3.2.1]oct-2-ene-8-carboxylate (155 mg, 0.463 mmol, Pharmablock), 4-(2-(((2R,7aS)-2-fluorotetrahydro-1H-pyrrolizin-7a(5H)-yl)methoxy)-4-(methylthio)quinazolin-7-yl)naphthalen-2-ol (100 mg, 0.210 mmol) and 1,4-dioxane. The vial was purged with nitrogen for 5 minutes and placed in a preheated aluminum block kept at 50° C. for 12 h. Upon completion, the reaction was cooled to room temperature and concentrated under reduced pressure to afford a crude black oil. The oil was then purified by column chromatography on silica gel, eluting with a gradient of 0-50%(3:1 EtOAc:EtOH with 2% TEA) in heptane to provide tert-butyl 3-(2-(((2R,7aS)-2-fluorotetrahydro-1H-pyrrolizin-7a(5H)-yl)methoxy)-7-(3-hydroxynaphthalen-1-yl)quinazolin-4-yl)-8-azabicyclo[3.2.1]oct-2-ene-8-carboxylate as a red oil.

The resulting red oil was dissolved in DCM (1 mL) and TFA (1.62 mL, 21.03 mmol, Aldrich) was added. The reaction was allowed to stir at 50° C. for 30 minutes. Upon completion the mixture was concentrated, redissolved in DMSO, filtered, and purified via reverse phase HPLC to give 4-(4-(8-azabicyclo[3.2.1]oct-2-en-3-yl)-2-(((2R,7aS)-2-fluorotetrahydro-1H-pyrrolizin-7a(5H)-yl)methoxy)quinazolin-7-yl)naphthalen-2-ol as a bis(2,2,2-trifluoroacetate) and orange solid. m/z (ESI, +ve ion): 537.2 (M+H)⁺. ¹H NMR (400 MHz, DMSO-d₆) δ ppm 8.42 (d, J=8.6 Hz, 1H), 7.77-7.93 (m, 3H), 7.71 (dd, J=8.6, 1.7 Hz, 1H), 7.63 (d, J=8.8 Hz, 1H), 7.45-7.51 (m, 1H), 7.25-7.31 (m, 2H), 7.13 (d, J=2.3 Hz, 1H), 6.59 (br d, J=5.6 Hz, 1H), 5.51-5.72 (m, 1H), 4.61-4.74 (m, 2H), 4.51-4.58 (m, 1H), 4.38 (br s, 1H), 3.56-3.96 (m, 7H), 3.17-3.24 (m, 1H), 2.76 (br d, J=18.2 Hz, 1H), 2.60-2.69 (m, 1H), 2.15-2.40 (m, 7H).

### 4-(4-((1S,4S)-2,5-Diazabicyclo[2.2.2]octan-2-yl)-6,8-difluoro-2-(((2R,7aS)-2-fluorotetrahydro-1H-pyrrolizin-7a(5H)-yl)methoxy)quinazolin-7-yl)-5-ethylnaphthalen-2-ol (Example 109

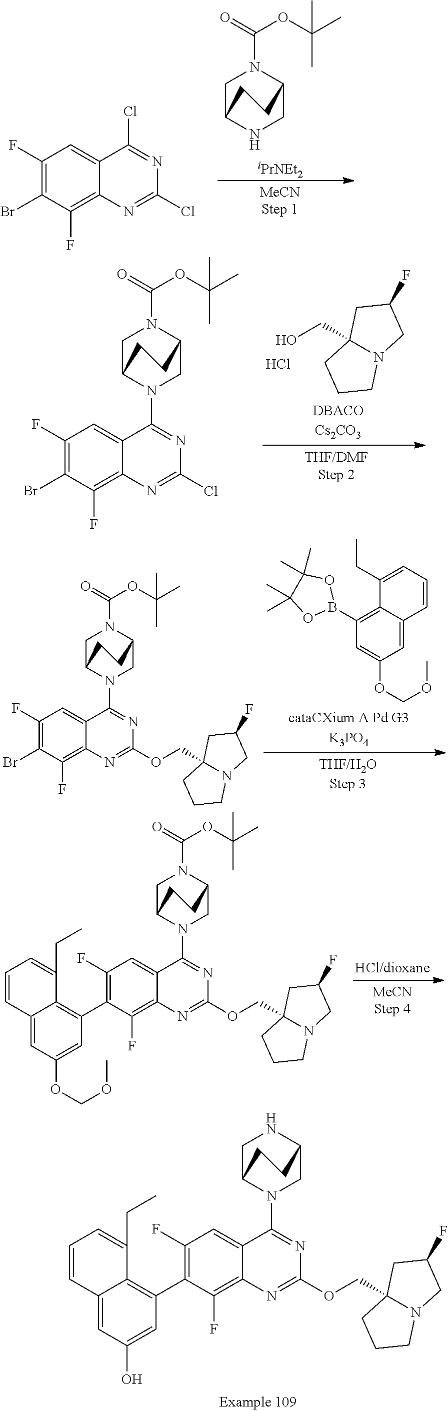

Step 1: tert-Butyl (1S,4S)-5-(7-bromo-2-chloro-6,&-difluoroquinazolin-4-yl)-2,5-diazabicyclo[2.2.2]octane-2-carboxylate. To a stirring solution of 7-bromo-2,4-dichloro-6,8-difluoroquinazolin (0.50 g, 1.59 mmol, Enamine) in acetonitrile (8.0 mL) was added (1S,4S)-2-Boc-2,5-diazabicyclo(2.2.2)octane (0.36 g, 1.67 mmol, AstaTech, Inc) and N,N-dimethyltriethylamine (0.62 mg, 0.8 mL, 4.78 mmol, Sigma-Aldrich Corporation). The reaction was then stirred for 1 h. The reaction was then diluted with water (10 mL) and brine (10 mL). The resulting aqueous layer was then extracted with CH₂Cl₂ (3×20 mL). The combined organic layers were dried over anhydrous sodium sulfate, filtered and concentrated under reduced pressure to provide tert-butyl (1S,4S)-5-(7-bromo-2-chloro-6,8-difluoroquinazolin-4-yl)-2,5-diazabicyclo[2.2.2]octane-2-carboxylate (0.78 g, 1.59 mmol, 100% yield) as a light yellow solid, which was used without further purification. m/z (ESI, +ve ion): 488.8 (M+H)⁺.

Step 2: tert-Butyl (1S,4S)-5-(7-bromo-6,8-difluoro-2-(((2R,7aS)-2-fluorotetrahydro-1H-pyrrolizin-7a(5H)-yl)methoxy)quinazolin-4-yl)-2,5-diazabicyclo[2.2.2]octane-2-carboxylate. A scintillation vial was charged with tert-butyl (1S,4S)-5-(7-bromo-2-chloro-6,8-difluoroquinazolin-4-yl)-2,5-diazabicyclo[2.2.2]octane-2-carboxylate (0.78 g, 1.59 mmol), cesium carbonate (1.60 g, 4.77 mmol, Sigma-Aldrich Corporation), 1,4-diazabicyclo[2.2.2]octane (36 mg, 0.32 mmol, Sigma-Aldrich Corporation), and ((2R,7aS)-2-fluorotetrahydro-1H-pyrrolizin-7a(5H)-yl)methanol (0.36 g, 2.23 mmol, PharmaBlock). Solids were then suspended in N,N-dimethylformamide (5.3 mL) and tetrahydrofuran (10.6 mL), and the reaction mixture was stirred at 35° C. After stirring for 2-d, the reaction was diluted with water (25 mL) and transferred to a separatory funnel. The layers were separated and the aqueous layer was extracted with EtOAc (3×25 mL). The organic layers were combined, dried over anhydrous sodium sulfate, filtered, and concentrated under reduced pressure. The crude reaction mixture was then purified by column chromatography on silica gel, eluting with a gradient of 0-50% 3:1 EtOAc/EtOH blend with 2% triethylamine in heptane to provide tert-butyl (1S,4S)-5-(7-bromo-6,8-difluoro-2-(((2R,7aS)-2-fluorotetrahydro-1H-pyrrolizin-7a(5H)-yl)methoxy)quinazolin-4-yl)-2,5-diazabicyclo[2.2.2]octane-2-carboxylate (0.84 g, 1.37 mmol, 86% yield) as a yellow solid. m/z (ESI, +ve ion): 612.2 (M+H)⁺.

Step 3: tert-Butyl (1S,4S)-5-(7-(8-ethyl-3-(methoxymethoxy)naphthalen-1-yl)-6,8-difluoro-2-(((2R,7aS)-2-fluorotetrahydro-1H-pyrrolizin-7a(5H)-yl)methoxy)quinazolin-4-yl)-2,5-diazabicyclo[2.2.2]octane-2-carboxylate. A vial was charged with tert-butyl (1S,4S)-5-(7-bromo-6,8-difluoro-2-(((2R,7aS)-2-fluorotetrahydro-1H-pyrrolizin-7a(5H)-yl)methoxy)quinazolin-4-yl)-2,5-diazabicyclo[2.2.2]octane-2-carboxylate (65 mg, 0.11 mmol), 2-(8-ethyl-3-(methoxymethoxy)naphthalen-1-yl)-4,4,5,5-tetramethyl-1,3,2-dioxaborolane (65 mg, 0.19 mmol, LabNetwork), potassium phosphate (68 mg, 0.32 mmol, Sigma Aldrich corporation), and cataCXium A Pd G3 (12 mg, 0.016 mmol, Sigma Aldrich Corporation). The vial was purged with nitrogen and the reactants were suspended in degassed tetrahydrofuran (1.0 mL) and water (0.1 mL). The reaction was then sealed and heated to 60° C. After stirring overnight, the reaction was cooled to room temperature and concentrated under reduced pressure. The crude oil was then purified via column chromatography on silica gel, eluting with a gradient of 0-50% 3:1 EtOAc/EtOH (with 2% triethylamine) in heptane to provide tert-butyl (1S,4S)-5-(7-(8-ethyl-3-(methoxymethoxy)naphthalen-1-yl)-6,8-difluoro-2-(((2R,7aS)-2-fluorotetrahydro-1H-pyrrolizin-7a(5H)-yl)methoxy)quinazolin-4-yl)-2,5-diazabicyclo[2.2.2]octane-2-carboxylate as off-white solid, which was used directly in the next step. m/z (ESI, +ve ion): 748.2 (M+H)⁺.

Step 4: 4-(4-((1S,4S)-2,5-Diazabicyclo[2.2.2]octan-2-yl)-6,8-difluoro-2-(((2R,7aS)-2-fluorotetrahydro-1H-pyrrolizin-7a(5H)-yl)methoxy)quinazolin-7-yl)-5-ethylnaphthalen-2-ol. The above tert-Butyl (1S,4S)-5-(7-(8-ethyl-3-(methoxymethoxy)naphthalen-1-yl)-6,8-difluoro-2-(((2R,7aS)-2-fluorotetrahydro-1H-pyrrolizin-7a(5H)-yl)methoxy)quinazolin-4-yl)-2,5-diazabicyclo[2.2.2]octane-2-carboxylate product was dissolved in MeCN (3.3 mL). The solution was cooled to 0° C. before HCl (4 M in 1,4-dioxane, 0.7 mL, 2.65 mmol, Sigma-Aldrich Corporation) was added. The reaction was stirred for 15 min. After an additional 15 min at rt, the reaction was concentrated under reduced pressure to provide a crude orange oil. The oil was then purified by reverse phase HPLC to provide 4-(4-((1S,4S)-2,5-diazabicyclo[2.2.2]octan-2-yl)-6,8-difluoro-2-(((2R,7aS)-2-fluorotetrahydro-1H-pyrrolizin-7a(5H)-yl)methoxy)quinazolin-7-yl)-5-ethylnaphthalen-2-ol as a bis(2,2,2-trifluoroacetate) and light yellow solid (61 mg, 0.073 mmol, 69% yield). m/z (ESI, +ve ion): 604.2 (M+H)⁺. ¹H NMR (400 MHz, METHANOL-d₄) δ ppm 7.84 (dd, J=10.14, 1.57 Hz, 1H) 7.61-7.69 (m, 1H) 7.34-7.41 (m, 1H) 7.32 (d, J=2.72 Hz, 1H) 7.18 (d, J=6.27 Hz, 1H) 6.95 (d, J=2.51 Hz, 1H) 5.46-5.71 (m, 1H) 5.12-5.24 (m, 1H) 4.60-4.73 (m, 2H) 4.50-4.59 (m, 1H) 4.35-4.45 (m, 1H) 3.83-4.09 (m, 5H) 3.56-3.63 (m, 1H) 3.43-3.53 (m, 1H) 2.58-2.81 (m, 2H) 2.42-2.57 (m, 4H) 2.33-2.42 (m, 2H) 2.08-2.29 (m, 4H) 0.93 (td, J=7.37, 3.45 Hz, 3H).

TABLE 3
Additional Examples. Prepared in an Analogous Manner to Example 109.
Products were isolated as corresponding TFA salts.

| | | | MS | |
|---|---|---|---|---|
| | | | m/z | |
| | | | (ESI, | |
| | | | +ve | |
| Ex.# | Structure | Name | ion) | ¹H NMR |
| 110 | 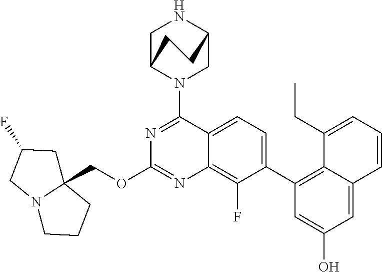 [SMILES: CCc1cccc2cc(O)cc(-c3ccc4c(N5C[C@@H]6CC[C@H]5CN6)nc(OC[C@@]56CCCN5C[C@H](F)C6)nc4c3F)c12] | 4-(4-((1S,4S)-2,5- diazabicyclo[2.2.2] octan-2-yl)-8- fluoro-2- (((2R,7aS)-2- fluorotetrahydro- 1H-pyrrolizin- 7a(5H)-yl)methoxy) quinazolin- 7-yl)-5- ethylnaphthalen-2- ol | 586.2 (M + H)⁺ | ¹H NMR (400 MHz, METHANOL-d₄) δ ppm 8.05 (d, J = 8.6 Hz, 1 H), 7.63 (d, J = 7.7 Hz, 1 H), 7.40 − 7.47 (m, 1 H), 7.32 − 7.39 (m, 1 H), 7.24 − 7.30 (m, 1 H), 7.13 − 7.19 (m, 1 H), 6.89 − 6.94 (m, 1 H), 5.44 − 5.72 (m, 1 H), 5.18 − 5.25 (m, 1 H), 4.67 − 4.73 (m, 2 H), 4.51 − 4.60 (m, 1 H), 4.36 − 4.46 (m, 1 H), 3.78 − 4.11 (m, 5 H), 3.55 − 3.65 (m, 1 H), 3.39 − 3.52 (m, 1 H), 2.11 − 2.77 (m, 12 H), 0.87 (br d, J = 3.8 Hz, 3 H). |
| 111 | 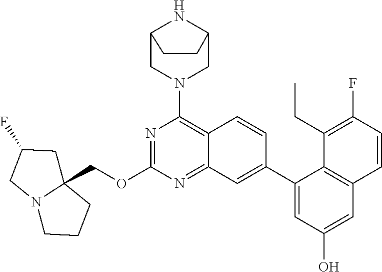 [SMILES: CCc1c(F)ccc2cc(O)cc(-c3ccc4c(N5CC6CCC(C5)N6)nc(OC[C@@]56CCCN5C[C@H](F)C6)nc4c3)c12] | 4-(4-((1R,5S)-3,8- diazabicyclo[3.2.1 octan-3-yl)-2- (((2R,7aS)-2- fluorotetrahydro- 1H-pyrrolizin- 7a(5H)-yl)methoxy) quinazolin- 7-yl)-5-ethyl- 6- fluoronaphthalen- 2-ol | 586.3 (M + H)⁺ | ¹H NMR (400 MHz, DMSO-d₆) δ ppm 8.06 (d, J = 8.5 Hz, 1 H), 7.76 (dd, J = 9.2, 6.1 Hz, 1 H), 7.62 (d, J = 1.7 Hz, 1 H), 7.32 − 7.44 (m, 2 H), 7.27 (d, J = 2.5 Hz, 1 H), 6.88 (d, J = 2.5 Hz, 1 H), 5.47 − 5.70 (m, 1 H), 4.57 − 4.66 (m, 2 H), 4.43 (br d, J = 6.6 Hz, 2 H), 4.17 − 4.26 (m, 3 H), 3.76 − 3.92 (m, 7 H), 3.66 − 3.72 (m, 2 H), 3.32 (br d, J = 5.8 Hz, 1 H), 1.94 − 2.26 (m, 9 H), 0.71 (t, J = 7.4 Hz, 3 H). |
| 112 | 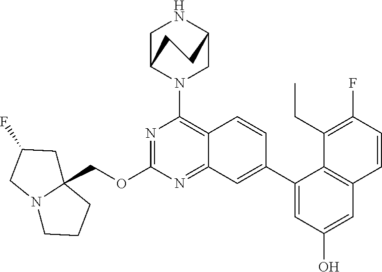 [SMILES: CCc1c(F)ccc2cc(O)cc(-c3ccc4c(N5C[C@@H]6CC[C@H]5CN6)nc(OC[C@@]56CCCN5C[C@H](F)C6)nc4c3)c12] | 4-(4-((1S,4S)-2,5- diazabicyclo[2.2.2] octan-2-yl)-2- (((2R,7aS)-2- fluorotetrahydro- 1H-pyrrolizin- 7a(5H)- yl)methoxy) quinazolin- 7-yl)-5-ethyl-6- fluoronaphthalen- 2-ol | 586.3 (M + H)⁺ | ¹H NMR (400 MHz, DMSO-d₆) δ ppm 8.13 (br d, J = 8.7 Hz, 1 H), 7.76 (dd, J = 9.1, 6.0 Hz, 1 H), 7.59 (t, J = 2.1 Hz, 1 H), 7.31 − 7.42 (m, 2 H), 7.27 (d, J = 2.5 Hz, 1 H), 6.87 (d, J = 1.9 Hz, 1 H), 5.48 − 5.68 (m, 1 H), 4.96 (br s, 1 H), 4.58 (s, 2 H), 4.19 − 4.39 (m, 3 H), 3.81 − 3.95 (m, 7 H), 3.27 − 3.61 (m, 6 H), 1.91 − 2.22 (m, 7 H), 0.73 (t, J = 7.0 Hz, 3 H). |
| 113 | 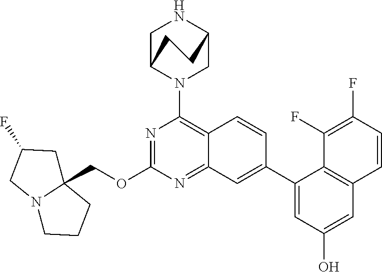 [SMILES: Oc1cc(-c2ccc3c(N4C[C@@H]5CC[C@H]4CN5)nc(OC[C@@]45CCCN4C[C@H](F)C5)nc3c2)c2c(F)c(F)ccc2c1] | 4-(4-((1S,4S)-2,5- diazabicyclo[2.2.2] octan-2-yl)-2- (((2R,7aS)-2- fluorotetrahydro- 1H-pyrrolizin- 7a(5H)- yl)methoxy) quinazolin- 7-yl)-5,6- difluoronaphthalen- 2-ol | 576.3 (M + H)⁺ | ¹H NMR (400 MHz, DMSO-d₆) δ ppm 8.11 (d, J = 8.7 Hz, 1 H), 7.69 − 7.78 (m, 1 H), 7.53 − 7.63 (m, 2 H), 7.44 (br d, J = 8.3 Hz, 1 H), 7.34 (t, J = 2.1 Hz, 1 H), 7.05 (d, J = 2.3 Hz, 1 H), 5.47 − 5.69 (m, 1 H), 4.96 (br s, 1 H), 4.59 (s, 2 H), 4.23 − 4.39 (m, 2 H), 3.87 − 3.95 (m, 3 H), 3.26 − 3.54 (m, 7 H), 1.88 − 2.32 (m, 9 H). |
| 114 | 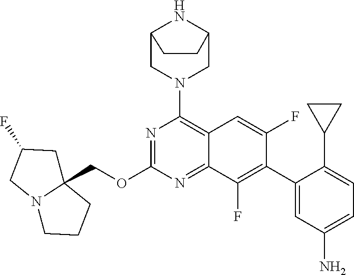 [SMILES: Nc1ccc(C2CC2)c(-c2c(F)cc3c(N4CC5CCC(C4)N5)nc(OC[C@@]45CCCN4C[C@H](F)C5)nc3c2F)c1] | 3-(4-((1R,5S)-3,8- diazabicyclo[3.2.1] octan-3-yl)-6,8- difluoro-2- (((2R,7aS)-2- fluorotetrahydro- 1H-pyrrolizin- 7a(5H)- yl)methoxy) quinazolin-7-yl)-4- cyclopropylaniline | 565.3 (M + H)⁺ | ¹H NMR (400 MHz, DMSO-d₆) δ ppm 7.75 (d, J = 9.8 Hz, 1 H), 6.88 (d, J = 8.2 Hz, 1 H), 6.74 (br d, J = 8.6 Hz, 1 H), 6.55 (br s, 1 H), 5.51 − 5.70 (m, 1 H), 4.58 (s, 2 H), 4.47 (br t, J = 14.1 Hz, 2 H), 4.19 (br s, 3 H), 3.71 − 3.82 (m, δ H), 3.33 (br d, J = 1.0 Hz, 1 H), 1.93 − 2.32 (m, 9 H), 1.40 − 1.49 (m, 1 H), 0.56 − 0.63 (m, 2 H), 0.43 − 0.56 (m, 2 H). |
| 115 | 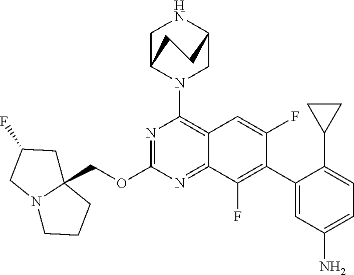 [SMILES: Nc1ccc(C2CC2)c(-c2c(F)cc3c(N4C[C@@H]5CC[C@H]4CN5)nc(OC[C@@]45CCCN4C[C@H](F)C5)nc3c2F)c1] | 3-(4-((1S,4S)-2,5- diazabicyclo[2.2.2] octan-2-yl)-6,8- difluoro-2- (((2R,7aS)-2- fluorotetrahydro- 1H-pyrrolizin- 7a(5H)- yl)methoxy) quinazolin-7-yl)-4- cyclopropylaniline | 565.3 (M + H)⁺ | ¹H NMR (400 MHz, DMSO-d₆) δ ppm 7.80 (d, J = 10.7 Hz, 1 H), 6.90 (dd, J = 8.4, 4.6 Hz, 1 H), 6.76 (br d, J = 8.8 Hz, 1 H), 6.57 (br s, 1 H), 5.50 − 5.69 (m, 1 H), 4.94 (br d, J = 2.1 Hz, 1 H), 4.57 (s, 2 H), 4.20 − 4.36 (m, 3 H), 3.79 − 3.93 (m, 10 H), 1.87 − 2.30 (m, 9 H), 1.45 (br dd, J = 7.9, 5.9 Hz, 1 H), 0.55 − 0.63 (m, 2 H), 0.42 − 0.53 (m, 2 H). |
| 116 | 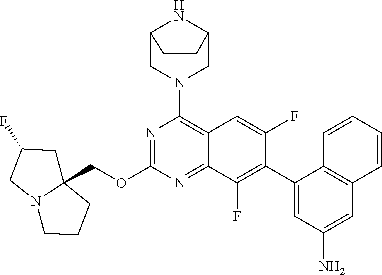 [SMILES: Nc1cc(-c2c(F)cc3c(N4CC5CCC(C4)N5)nc(OC[C@@]45CCCN4C[C@H](F)C5)nc3c2F)c2ccccc2c1] | 4-(4-((1R,5S)-3,8- diazabicyclo[3.2.1] octan-3-yl)-6,8- difluoro-2- (((2R,7aS)-2- fluorotetrahydro- 1H-pyrrolizin- 7a(5H)- yl)methoxy) quinazolin-7- yl)naphthalen-2- amine | 575.3 (M + H)⁺ | ¹H NMR (400 MHz, DMSO-d₆) δ ppm 7.83 (d, J = 10.0 Hz, 1 H), 7.67 (d, J = 8.2 Hz, 1 H), 7.37 (ddd, J = 8.2, 6.8, 1.0 Hz, 1 H), 7.15 − 7.23 (m, 1 H), 7.01 − 7.14 (m, 3 H), 5.48 − 5.71 (m, 1 H), 4.49 − 4.64 (m, 4 H), 4.20 (br s, 2 H), 3.78 − 3.93 (m, 9 H), 3.26 − 3.37 (m, 2 H), 2.32 − 2.39 (m, 1 H), 2.14 − 2.26 (m, 2 H), 1.99 (br s, 5 H). |
| 117 | 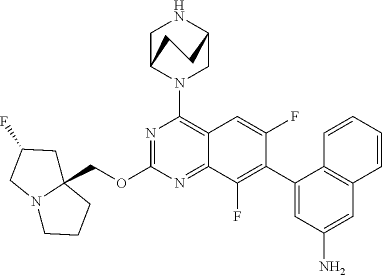 [SMILES: Nc1cc(-c2c(F)cc3c(N4C[C@@H]5CC[C@H]4CN5)nc(OC[C@@]45CCCN4C[C@H](F)C5)nc3c2F)c2ccccc2c1] | 4-(4-((1S,4S)-2,5- diazabicyclo[2.2.2] octan-2-yl)-6,8- difluoro-2- (((2R,7aS)-2- fluorotetrahydro- 1H-pyrrolizin- 7a(5H)- yl)methoxy) quinazolin-7- yl)naphthalen-2- amine | 575.3 (M + H)⁺ | ¹H NMR (400 MHz, DMSO-d₆) δ ppm 7.88 (br dd, J = 10.5, 2.3 Hz, 1 H), 7.68 (d, J = 8.4 Hz, 1 H), 7.37 (ddd, J = 8.2, 6.6, 1.4 Hz, 1 H), 7.01 − 7.21 (m, 4 H), 5.47 − 5.68 (m, 1 H), 4.98 (br s, 1 H), 4.60 (d, J = 2.5 Hz, 2 H), 4.28 − 4.43 (m, 2 H), 3.92 (br s, 6 H), 3.48 (br s, 3 H), 3.26 − 3.39 (m, 2 H), 1.90 − 2.37 (m, 9 H). |
| 118 | 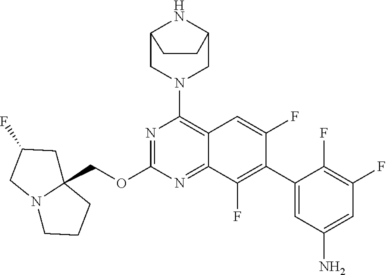 [SMILES: Nc1cc(F)c(F)c(-c2c(F)cc3c(N4CC5CCC(C4)N5)nc(OC[C@@]45CCCN4C[C@H](F)C5)nc3c2F)c1] | 3-(4-((1R,5S)-3,8- diazabicyclo[3.2.1 octan-3-yl)-6,8- difluoro-2- (((2R,7aS)-2- fluorotetrahydro- 1H-pyrrolizin- 7a(5H)-yl)methoxy) quinazolin- 7-yl)-4,5- difluoroaniline | 561.2 (M + H)⁺ | ¹H NMR (400 MHz, DMSO-d₆) δ ppm 7.75 − 7.86 (m, 1 H), 6.70 (ddd, J = 13.0, 6.7, 2.7 Hz, 1 H), 6.41 (br s, 1 H), 5.51 − 5.69 (m, 1 H), 4.41 − 4.67 (m, 5 H), 4.18 (brs, 3 H), 3.86 (br d, J = 17.8 Hz, 5 H), 3.70 − 3.73 (m, 2 H), 3.33 (br d, J = 6.3 Hz, 1 H), 1.86 − 2.33 (m, 9 H). |
| 119 | 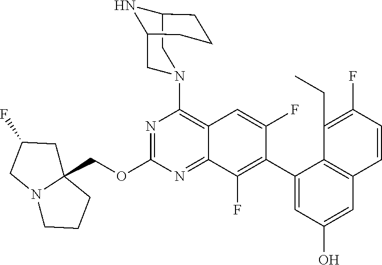 [SMILES: CCc1c(F)ccc2cc(O)cc(-c3c(F)cc4c(N5CC6CCCC(C5)N6)nc(OC[C@@]56CCCN5C[C@H](F)C6)nc4c3F)c12] | 4-(4-((1R,5S)-3,9- diazabicyclo[3.3.1] nonan-3-yl)-6,8- difluoro-2- (((2R,7aS)-2- fluorotetrahydro- 1H-pyrrolizin- 7a(5H)-yl)methoxy) quinazolin- 7-yl)-5-ethyl- 6- fluoronaphthalen- 2-ol | 636.3 (M + H)⁺ | ¹H NMR (METHANOL-d₄, 400 MHz) δ 7.6-7.8 (m, 2H), 7.2-7.4 (m, 2H), 7.00 (br s, 1H), 5.5-5.7 (m, 1H), 4.71 (br s, 3H), 4.7-4.7 (m, 1H), 4.7-4.8 (m, 1H), 4.7-4.7 (m, 1H), 4.5-4.5 (m, 1H), 3.8-4.0 (m, 6H), 3.8-4.2 (m, 1H), 3.50 (br s, 1H), 2.5-2.7 (m, 2H), 2.3-2.5 (m, 4H), 2.2-2.3 (m, 6H), 0.81 (br d, 3H, J = 7.1 Hz). |
| 120 | 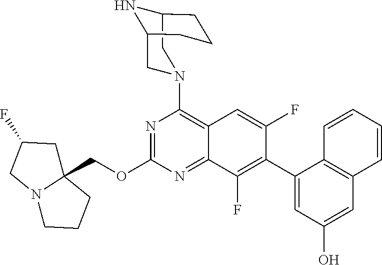 [SMILES: Oc1cc(-c2c(F)cc3c(N4CC5CCCC(C4)N5)nc(OC[C@@]45CCCN4C[C@H](F)C5)nc3c2F)c2ccccc2c1] | 4-(4-((1R,5S)-3,9- diazabicyclo[3.3.1] nonan-3-yl)-6,8- difluoro-2- (((2R,7aS)-2- fluorotetrahydro- 1H-pyrrolizin- 7a(5H)- yl)methoxy) quinazolin-7- yl)naphthalen-2-ol | 590.2 (M + H)⁺ | ¹H NMR (METHANOL-d₄, 400 MHz) δ 7.8-7.9 (m, 2H), 7.47 (ddd, 1H, J = 1.0, 6.8, 8.2 Hz), 7.37 (br d, 1H, J = 7.9 Hz), 7.33 (d, 1H, J = 2.5 Hz), 7.2-7.3 (m, 1H), 7.1-7.2 (m, 1H), 5.5-5.7 (m, 1H), 4.97 (br dd, 2H, J = 7.8, 13.7 Hz), 4.7-4.8 (m, 2H), 3.9-4.1 (m, 7H), 3.5-3.5 (m, 1H), 2.74 (br t, 1H, J = 3.7 Hz), 2.6-2.8 (m, 1H), 2.3-2.5 (m, 4H), 2.1-2.3 (m, 6H). |
| 121 | 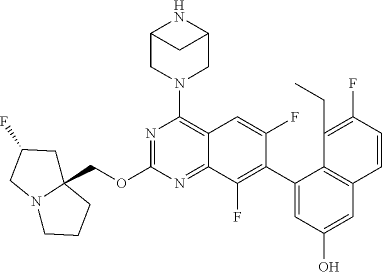 [SMILES: CCc1c(F)ccc2cc(O)cc(-c3c(F)cc4c(N5CC6CC(C5)N6)nc(OC[C@@]56CCCN5C[C@H](F)C6)nc4c3F)c12] | 4-(4-((1R,5S)-3,6- diazabicyclo[3.1.1] heptan-3-yl)-6,8- difluoro-2- (((2R,7aS)-2- fluorotetrahydro- 1H-pyrrolizin- 7a(5H)- yl)methoxy) quinazolin-7- yl)-5-ethyl-6- fluoronaphthalen- 2-ol | 608.2 (M + H)⁺ | ¹H NMR (METHANOL-d₄, 400 MHz) δ 7.6-7.8 (m, 2H), 7.2-7.4 (m, 2H), 6.99 (s, 1H), 5.5-5.7 (m, 1H), 5.2-5.3 (m, 2H), 4.69 (d, 2H, J = 2.1 Hz), 3.8-4.1 (m, 6H), 3.71 (br d, 2H, J = 13.4 Hz), 3.4-3.6 (m, 1H), 2.8-2.8 (m, 1H), 2.5-2.7 (m, 2H), 2.5-2.8 (m, 1H), 2.3-2.5 (m, 4H), 2.1-2.3 (m, 1H), 0.83 (t, 3H, J = 7.3 Hz). |
| 122 | 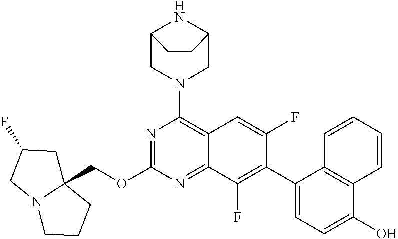 [SMILES: Oc1ccc(-c2c(F)cc3c(N4CC5CCC(C4)N5)nc(OC[C@@]45CCCN4C[C@H](F)C5)nc3c2F)c2ccccc12] | 4-(4-((1R,5S)-3,8- diazabicyclo[3.2.1] octan-3-yl)-6,8- difluoro-2- (((2R,7aS)-2- fluorotetrahydro- 1H-pyrrolizin- 7a(5H)- yl)methoxy) quinazolin-7- yl)naphthalen-1-ol | 576.2 (M + H)⁺ | ¹H NMR (METHANOL-d₄, 400 MHz) δ 8.31 (d, 1H, J = 8.2 Hz), 7.58 (dd, 1H, J = 1.8, 10.1 Hz), 7.4- 7.5 (m, 1H), 7.40 (d, 2H, J = 4.0 Hz), 7.30 (d, 1H, J = 7.7 Hz), 6.94 (d, 1H, J = 7.9 Hz), 5.1-5.4 (m, 1H), 4.4-4.5 (m, 2H), 4.2-4.3 (m, 1H), 4.2-4.2 (m, 1H), 3.5-3.7 (m, 4H), 3.1-3.3 (m, 3H), 3.00 (dt, 1H, J = 5.9, 9.3 Hz), 2.1-2.4 (m, 3H), 1.9-2.0 (m, 2H), 1.8-1.9 (m, 5H). |
| 123 | 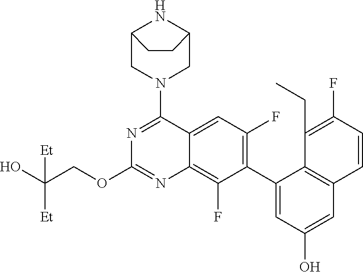 [SMILES: CCc1c(F)ccc2cc(O)cc(-c3c(F)cc4c(N5CC6CCC(C5)N6)nc(OCC(O)(CC)CC)nc4c3F)c12] | 4-(4-((1R,5S)-3,8- diazabicyclo[3.2.1] octan-3-yl)-2-(2- ethyl-2- hydroxybutoxy)- 6,8- difluoroquinazolin- 7-yl)-5-ethyl-6- fluoronaphthalen- 2-ol | 581.2 (M + H)⁺ | ¹H NMR (METHANOL-d₄, 400 MHz) δ 7.66 (dd, 1H, J = 5.9, 9.0 Hz), 7.60 (dd, 1H, J = 1.7, 10.0 Hz), 7.29 (d, 1H, J = 2.7 Hz), 7.24 (t, 1H, J = 9.3 Hz), 6.98 (d, 1H, J = 2.5 Hz), 4.45 (br t, 2H, J = 14.8 Hz), 4.32 (d, 2H, J = 1.3 Hz), 3.5-3.7 (m, 4H), 2.5-2.7 (m, 1H), 2.4-2.5 (m, 1H), 1.86 (br s, 4H), 1.67 (q, 4H, J = 7.5 Hz), 0.95 (t, 6H, J = 7.5 Hz), 0.81 (t, 3H, J = 7.4 Hz). |
| 124 | 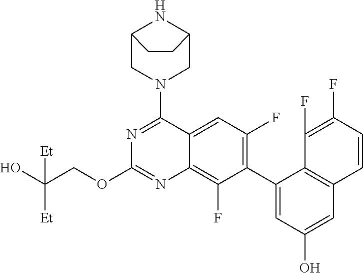 [SMILES: CCC(O)(CC)COc1nc(N2CC3CCC(C2)N3)c2cc(F)c(-c3cc(O)cc4ccc(F)c(F)c34)c(F)c2n1] | 4-(4-((1R,5S)-3,8- diazabicyclo[3.2.1] octan-3-yl)-2-(2- ethyl-2- hydroxybutoxy)- 6,8- difluoroquinazolin- 7-yl)-5,6- difluoronaphthalen- 2-ol | 571.2 (M + H)⁺ | ¹H NMR (METHANOL-d₄, 400 MHz) δ 7.5-7.6 (m, 2H), 7.38 (dt, 1H, J = 7.6, 9.6 Hz), 7.31 (t, 1H, J = 2.2 Hz), 7.15 (d, 1H, J = 2.3 Hz), 4.4-4.5 (m, 2H), 4.33 (s, 2H), 3.5- 3.7 (m, 4H), 1.86 (br s, 4H), 1.6- 1.7 (m, 4H), 0.95 (t, 6H, J = 7.5 Hz). |
| 125 | 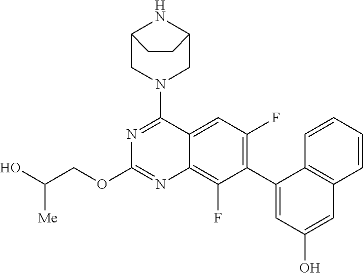 [SMILES: CC(O)COc1nc(N2CC3CCC(C2)N3)c2cc(F)c(-c3cc(O)cc4ccccc34)c(F)c2n1] | 4-(4-((1R,5S)-3,8- diazabicyclo[3.2.1] octan-3-yl)-6,8- difluoro-2-(2- hydroxypropoxy) quinazolin-7- yl)naphthalen-2-ol | 493.2 (M + H)⁺ | ¹H NMR (METHANOL-d₄, 400 MHz) δ 7.77 (d, 1H, J = 8.2 Hz), 7.6-7.7 (m, 1H), 7.4-7.5 (m, 1H), 7.35 (d, 1H, J = 8.6 Hz), 7.29 (d, 1H, J = 2.1 Hz), 7.2-7.3 (m, 1H), 7.12 (d, 1H, J = 2.3 Hz), 4.66 (br t, 2H, J = 13.0 Hz), 4.39 (d, 1H, J = 5.4 Hz), 4.25 (br s, 2H), 4.1-4.2 (m, 1H), 3.7-3.9 (m, 3H), 2.1-2.3 (m, 4H), 1.2-1.4 (m, 3H). |
| 126 | 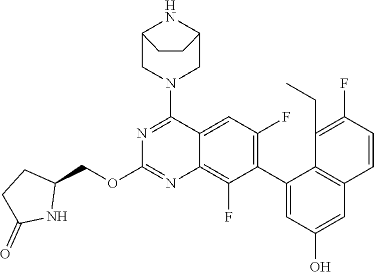 [SMILES: CCc1c(F)ccc2cc(O)cc(-c3c(F)cc4c(N5CC6CCC(C5)N6)nc(OC[C@@H]5CCC(=O)N5)nc4c3F)c12] | (5S)-5-(((4- ((1R,5S)-3,8- diazabicyclo[3.2.1] octan-3-yl)-7-(8- ethyl-7-fluoro-3- hydroxynaphthalen- 1-yl)-6,8- difluoroquinazolin- 2-yl)oxy)methyl) pyrrolidin-2-one | 578.2 (M + H)⁺ | ¹H NMR (400 MHz, METHANOL-d₄) δ ppm 7.66 − 7.73 (m, 2 H), 7.25 − 7.34 (m, 2 H), 6.99 (d, J = 2.7 Hz, 1 H), 4.55 − 4.70 (m, 3 H), 4.42 − 4.48 (m, 1 H), 4.27 (br s, 2 H), 4.11 − 4.17 (m, 1 H), 3.76 − 3.89 (m, 2 H), 2.56 − 2.63 (m, 1 H), 2.33 − 2.54 (m, 4 H), 2.15 − 2.30 (m, 4 H), 2.01 − 2.12 (m, 1 H), 0.83 (t, J = 7.4 Hz, 3 H). |
| 127 | 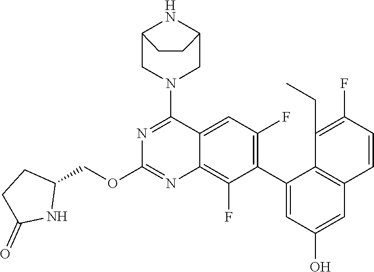 [SMILES: CCc1c(F)ccc2cc(O)cc(-c3c(F)cc4c(N5CC6CCC(C5)N6)nc(OC[C@H]5CCC(=O)N5)nc4c3F)c12] | (5R)-5-(((4- ((1R,5S)-3,8- diazabicyclo[3.2.1] octan-3-yl)-7-(8- ethyl-7-fluoro-3- hydroxynaphthalen- 1-yl)-6,8- difluoroquinazolin- 2- yl)oxy)methyl) pyrrolidin-2-one | 578.2 (M + H)⁺ | ¹H NMR (400 MHz, METHANOL-d₄) δ ppm 7.67 − 7.73 (m, 2 H), 7.25 − 7.34 (m, 2 H), 6.99 (d, J = 2.5 Hz, 1 H), 4.56 − 4.71 (m, 3 H), 4.42 − 4.48 (m, 1 H), 4.27 (br s, 2 H), 4.11 − 4.18 (m, 1 H), 3.77 − 3.90 (m, 2 H), 2.33 − 2.63 (m, 5 H), 2.15 − 2.30 (m, 4 H), 2.01 − 2.12 (m, 1 H), 0.83 (t, J = 7.4 Hz, 3 H). |
| 128 | 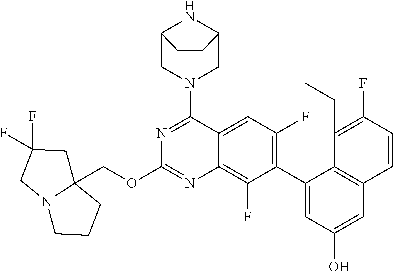 [SMILES: CCc1c(F)ccc2cc(O)cc(-c3c(F)cc4c(N5CC6CCC(C5)N6)nc(OCC56CCCN5CC(F)(F)C6)nc4c3F)c12] | 4-(4-((1R,5S)-3,8- diazabicyclo[3.2.1] octan-3-yl)-2- ((2,2- difluorotetrahydro- 1H-pyrrolizin- 7a(5H)- yl)methoxy)-6,8- difluoroquinazolin- 7-yl)-5-ethyl-6- fluoronaphthalen- 2-ol | 640.2 (M + H)⁺ | ¹H NMR (400 MHz, METHANOL-d₄) δ ppm 7.68 − 7.75 (m, 2 H), 7.34 (d, J = 2.7 Hz, 1 H), 7.28 (t, J = 9.3 Hz, 1 H), 7.00 (t, J = 2.0 Hz, 1 H), 4.60 − 4.75 (m, 4 H), 4.18 − 4.31 (m, 3 H), 3.81 − 3.99 (m, 4 H), 3.42 − 3.52 (m, 1 H), 2.94 − 3.07 (m, 1 H), 2.79 − 2.90 (m, 1 H), 2.55 − 2.62 (m, 1 H), 2.37 − 2.53 (m, 3 H), 2.17 − 2.36 (m, 6 H), 0.82 (t, J = 7.4 Hz, 3 H). |
| 129 | 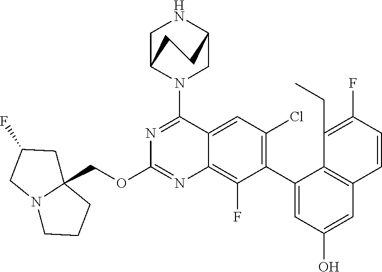 [SMILES: CCc1c(F)ccc2cc(O)cc(-c3c(Cl)cc4c(N5C[C@@H]6CC[C@H]5CN6)nc(OC[C@@]56CCCN5C[C@H](F)C6)nc4c3F)c12] | 4-(4-((1S,4S)-2,5- diazabicyclo[2.2.2] octan-2-yl)-6- chloro-8-fluoro-2- (((2R,7aS)-2- fluorotetrahydro- 1H-pyrrolizin- 7a(5H)-yl)methoxy) quinazolin- 7-yl)-5-ethyl- 6- fluoronaphthalen- 2-ol | 638.2 (M + H)⁺ | ¹H NMR (400 MHz, METHANOL-d₄) δ ppm 8.16 (t, J = 1.7 Hz, 1 H), 7.70 (dd, J = 9.2, 5.9 Hz, 1 H), 7.24 − 7.33 (m, 2 H), 6.90 (d, J = 2.5 Hz, 1 H), 5.44 − 5.73 (m, 1 H), 5.18 (br s, 1 H), 4.64 − 4.72 (m, 2 H), 4.54 (br d, J = 12.5 Hz, 1 H), 4.43 (ddd, J = 12.2, 5.5, 1.9 Hz, 1 H), 3.83 − 4.08 (m, 5 H), 3.61 (dd, J = 12.6, 1.8 Hz, 1 H), 3.44 − 3.53 (m, 1 H), 2.57 − 2.79 (m, 3 H), 2.22 − 2.50 (m, 6 H), 2.10 − 2.20 (m, 3 H), 0.82 (td, J = 7.4, 4.2 Hz, 3 H). |
| 130 | 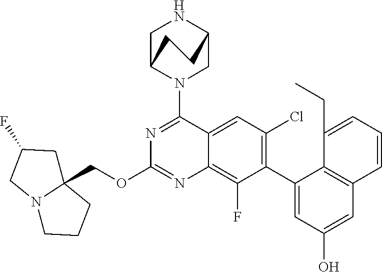 [SMILES: CCc1cccc2cc(O)cc(-c3c(Cl)cc4c(N5C[C@@H]6CC[C@H]5CN6)nc(OC[C@@]56CCCN5C[C@H](F)C6)nc4c3F)c12] | 4-(4-((1S,4S)-2,5- diazabicyclo[2.2.2] octan-2-yl)-6- chloro-8-fluoro-2- (((2R,7aS)-2- fluorotetrahydro- 1H-pyrrolizin- 7a(5H)-yl)methoxy) quinazolin- 7-yl)-5- ethylnaphthalen-2- ol | 620.2 (M + H)⁺ | ¹H NMR (400 MHz, METHANOL-d₄) δ ppm 8.14 (d, J = 1.3 Hz, 1 H), 7.65 (d, J = 7.5 Hz, 1 H), 7.38 (t, J = 7.3 Hz, 1 H), 7.30 (d, J = 2.7 Hz, 1 H), 7.16 (d, J = 6.9 Hz, 1 H), 6.85 (dd, J = 2.6, 0.9 Hz, 1 H), 5.46 − 5.69 (m, 1 H), 5.16 − 5.21 (m, 1 H), 4.65 − 4.74 (m, 2 H), 4.54 (dd, J = 12.3, 1.9 Hz, 1 H), 4.43 (br d, J = 12.5 Hz, 1 H), 3.83 − 4.07 (m, 5 H), 3.60 (dd, J = 12.5, 1.9 Hz, 1 H), 3.43 − 3.52 (m, 1 H), 2.56 − 2.81 (m, 2 H), 2.10 − 2.52 (m, 10 H), 0.94 (td, J = 7.4, 3.2 Hz, 3 H). |
| 131 | 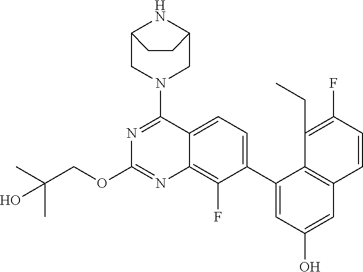 [SMILES: CCc1c(F)ccc2cc(O)cc(-c3ccc4c(N5CC6CCC(C5)N6)nc(OCC(C)(C)O)nc4c3F)c12] | 4-(4-((1R,5S)-3,8- diazabicyclo[3.2.1] octan-3-yl)-8- fluoro-2-(2- hydroxy-2- methylpropoxy) quinazolin-7-yl)-5- ethyl-6- fluoronaphthalen- 2-ol | 535.2 (M + H)⁺ | ¹H NMR (400 MHz, METHANOL-d₄) δ ppm 7.96 (d, J = 8.8 Hz, 1 H), 7.69 (dd, J = 9.0, 5.9 Hz, 1 H), 7.49 (dd, J = 8.6, 6.9 Hz, 1 H), 7.24 − 7.31 (m, 2 H), 6.97 (d, J = 2.7 Hz, 1 H), 4.82 − 4.84 (m, 2 H), 4.42 (s, 2 H), 4.31 (br s, 2 H), 3.95 (dd, J = 13.9, 8.7 Hz, 2 H), 2.39 − 2.53 (m, 2 H), 2.21 (s, 4 H), 1.38 (s, 6 H), 0.81 (t, J = 7.3 Hz, 3 H). |
| 132 | 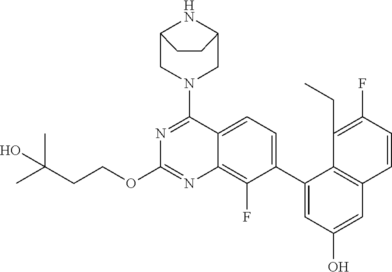 [SMILES: CCc1c(F)ccc2cc(O)cc(-c3ccc4c(N5CC6CCC(C5)N6)nc(OCCC(C)(C)O)nc4c3F)c12] | 4-(4-((1R,5S)-3,8- diazabicyclo[3.2.1] octan-3-yl)-8- fluoro-2-(3- hydroxy-3- methylbutoxy) quinazolin-7-yl)-5- ethyl-6- fluoronaphthalen- 2-ol | 549.2 (M + H)⁺ | ¹H NMR (400 MHz, METHANOL-d₄) δ ppm 7.97 (d, J = 8.2 Hz, 1 H), 7.69 (dd, J = 9.0, 5.9 Hz, 1 H), 7.50 (dd, J = 8.8, 6.9 Hz, 1 H), 7.24 − 7.30 (m, 2 H), 6.97 (d, J = 2.5 Hz, 1 H), 4.84 − 4.93 (m, 1 H), 4.72 − 4.80 (m, 3 H), 4.31 (br s, 2 H), 3.96 (br dd, J = 13.6, 6.1 Hz, 2 H), 2.40 − 2.53 (m, 2 H), 2.21 (s, 4 H), 2.05 − 2.10 (m, 2 H), 1.33 (s, 6 H), 0.82 (t, J = 7.4 Hz, 3 H). |
| 133 | 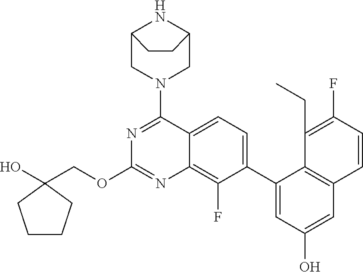 [SMILES: CCc1c(F)ccc2cc(O)cc(-c3ccc4c(N5CC6CCC(C5)N6)nc(OCC5(O)CCCC5)nc4c3F)c12] | 4-(4-((1R,5S)-3,8- diazabicyclo[3.2.1] octan-3-yl)-8- fluoro-2-((1- hydroxycyclopentyl) methoxy) quinazolin- 7-yl)-5-ethyl-6- fluoronaphthalen- 2-ol | 561.2 (M + H)⁺ | ¹H NMR (400 MHz, METHANOL-d₄) δ ppm 7.98 (d, J = 8.6 Hz, 1 H), 7.69 (dd, J = 9.2, 5.9 Hz, 1 H), 7.51 (dd, J = 8.6, 6.9 Hz, 1 H), 7.24 − 7.33 (m, 2 H), 6.98 (d, J = 2.7 Hz, 1 H), 4.68 − 4.77 (m, 2 H), 4.57 (s, 2 H), 4.32 (br s, 2 H), 3.98 (dd, J = 13.5, 9.3 Hz, 2 H), 2.46 (td, J = 7.6, 2.6 Hz, 2 H), 2.20 (s, 4 H), 1.70 − 1.96 (m, 8 H), 0.81 (t, J = 7.4 Hz, 3 H). |
| 134 | 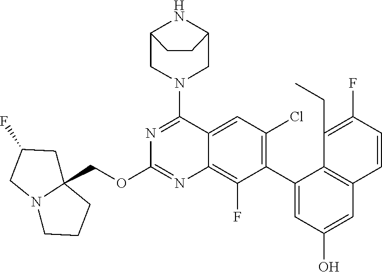 [SMILES: CCc1c(F)ccc2cc(O)cc(-c3c(Cl)cc4c(N5CC6CCC(C5)N6)nc(OC[C@@]56CCCN5C[C@H](F)C6)nc4c3F)c12] | 4-(4-((1R,5S)-3,8- diazabicyclo[3.2.1] octan-3-yl)-6- chloro-8-fluoro-2- (((2R,7aS)-2- fluorotetrahydro- 1H-pyrrolizin- 7a(5H)-yl)methoxy) quinazolin- 7-yl)-5-ethyl- 6- fluoronaphthalen- 2-ol | 538.2 (M + H)⁺ | ¹H NMR (400 MHz, METHANOL-d₄) δ ppm 8.02 (d, J = 1.3 Hz, 1 H), 7.70 (dd, J = 9.1, 6.0 Hz, 1 H), 7.23 − 7.37 (m, 2 H), 6.90 (d, J = 2.5 Hz, 1 H), 5.41 − 5.73 (m, 1 H), 4.64 − 4.78 (m, 4 H), 4.29 (br s, 2 H), 3.86 − 4.05 (m, 5 H), 3.45 − 3.56 (m, 1 H), 2.57 − 2.79 (m, 3 H), 2.30 − 2.48 (m, 4 H), 2.10 − 2.22 (m, 5 H), 0.81 (td, J = 7.4, 2.5 Hz, 3 H). |
| 135 | 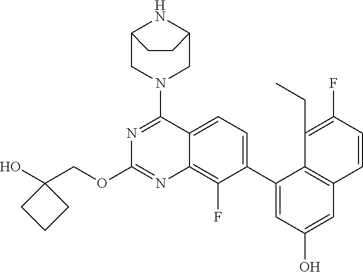 [SMILES: CCc1c(F)ccc2cc(O)cc(-c3ccc4c(N5CC6CCC(C5)N6)nc(OCC5(O)CCC5)nc4c3F)c12] | 4-(4-((1R,5S)-3,8- diazabicyclo[3.2.1] octan-3-yl)-8- fluoro-2-((1- hydroxycyclobutyl) methoxy)quinazol in-7-yl)-5-ethyl-6- fluoronaphthalen- 2-ol | 547.2 (M + H)⁺ | ¹H NMR (400 MHz, METHANOL-d₄) δ ppm 7.96 (d, J = 8.7 Hz, 1 H), 7.69 (dd, J = 9.0, 5.9 Hz, 1 H), 7.49 (dd, J = 8.6, 6.9 Hz, 1 H), 7.24 − 7.31 (m, 2 H), 6.97 (d, J = 2.5 Hz, 1 H), 4.83 − 4.88 (m, 1 H), 4.73 − 4.80 (m, 1 H), 4.62 (s, 2 H), 4.31 (br s, 2 H), 3.94 (dd, J = 13.7, 8.3 Hz, 2 H), 2.39 − 2.56 (m, 2 H), 2.13 − 2.34 (m, δ H), 1.81 − 1.91 (m, 1 H), 1.71 (dt, J = 11.4, 9.0 Hz, 1 H), 0.81 (t, J = 7.4 Hz, 3 H). |
| 136 | 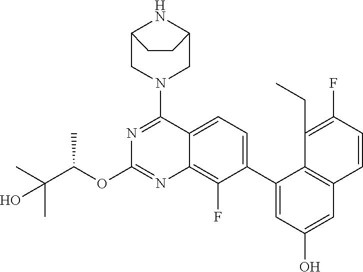 [SMILES: CCc1c(F)ccc2cc(O)cc(-c3ccc4c(N5CC6CCC(C5)N6)nc(O[C@@H](C)C(C)(C)O)nc4c3F)c12] | 4-(4-((1R,5S)-3,8- diazabicyclo[3.2.1] octan-3-yl)-8- fluoro-2-(((S)-3- hydroxy-3- methylbutan-2- yl)oxy)quinazolin- 7-yl)-5-ethyl-6- fluoronaphthalen- 2-ol | 549.2 (M + H)⁺ | ¹H NMR (400 MHz, METHANOL-d₄) δ ppm 7.91 (d, J = 8.4 Hz, 1 H), 7.68 (dd, J = 9.2, 5.9 Hz, 1 H), 7.43 (dd, J = 8.2, 7.1 Hz, 1 H), 7.24 − 7.29 (m, 2 H), 6.97 (d, J = 2.7 Hz, 1 H), 5.29 (qd, J = 6.4, 1.3 Hz, 1 H), 4.68 − 4.79 (m, 3 H), 4.29 (br s, 2 H), 3.86 (br dd, J = 14.2, 8.2 Hz, 2 H), 2.40 − 2.55 (m, 2 H), 2.16 − 2.29 (m, 4 H), 1.43 (dd, J = 6.4, 3.4 Hz, 3 H), 1.29 − 1.36 (m, 6 H), 0.81 (t, J = 7.4 Hz, 3 H). |
| 137 | 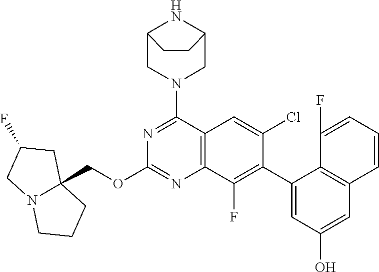 [SMILES: Oc1cc(-c2c(Cl)cc3c(N4CC5CCC(C4)N5)nc(OC[C@@]45CCCN4C[C@H](F)C5)nc3c2F)c2c(F)cccc2c1] | 4-(4-((1R,5S)-3,8- diazabicyclo[3.2.1] octan-3-yl)-6- chloro-8-fluoro-2- (((2R,7aS)-2- fluorotetrahydro- 1H-pyrrolizin- 7a(5H)-yl)methoxy) quinazolin-7-yl)-5- fluoronaphthalen- 2-ol | 610.2 (M + H)⁺ | ¹H NMR (400 MHz, METHANOL-d₄) δ ppm 7.97 (d, J = 1.7 Hz, 1 H), 7.61 (d, J = 7.9 Hz, 1 H), 7.31 − 7.45 (m, 2 H), 7.01 (s, 1 H), 6.86 − 6.93 (m, 1 H), 5.49 − 5.68 (m, 1 H), 4.72 − 4.79 (m, 6 H), 4.23 − 4.38 (m, 1 H), 3.78 − 4.12 (m, 4 H), 3.42 − 3.54 (m, 1 H), 2.65 (s, 1 H), 2.54 (br t, J = 1.9 Hz, 2 H), 2.30 − 2.43 (m, 2 H), 2.12 − 2.20 (m, 5 H). |
| 138 | 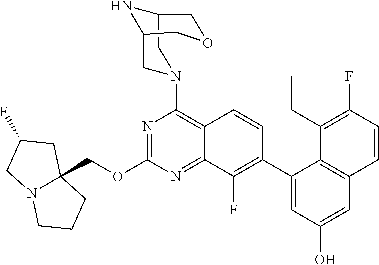 [SMILES: CCc1c(F)ccc2cc(O)cc(-c3ccc4c(N5CC6COCC(C5)N6)nc(OC[C@@]56CCCN5C[C@H](F)C6)nc4c3F)c12] | 4-(4-((1R,5S)-3- oxa-7,9- diazabicyclo[3.3.1] nonan-7-yl)-8- fluoro-2- (((2R,7aS)-2- fluorotetrahydro- 1H-pyrrolizin- 7a(5H)-yl)methoxy) quinazolin- 7-yl)-5-ethyl- 6- fluoronaphthalen- 2-ol | 620.2 (M + H)⁺ | ¹H NMR (400 MHz, METHANOL-d₄) δ ppm 8.01 − 8.06 (m, 1 H), 7.72 − 7.78 (m, 1 H), 7.50 − 7.57 (m, 1 H), 7.29 − 7.37 (m, 2 H), 7.02 − 7.05 (m, 1 H), 5.54 − 5.73 (m, 1 H), 5.23 − 5.33 (m, 2 H), 4.72 − 4.80 (m, 2 H), 4.09 − 4.36 (m, 7 H), 3.91 − 4.06 (m, 2 H), 3.81 (br s, 2 H), 3.49 − 3.61 (m, 1 H), 2.36 − 2.72 (m, 7 H), 2.17 − 2.30 (m, 1 H), 0.83 − 0.89 (m, 3 H). |
| 139 | 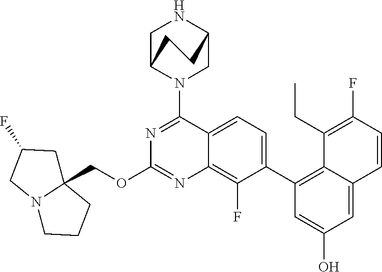 [SMILES: CCc1c(F)ccc2cc(O)cc(-c3ccc4c(N5C[C@@H]6CC[C@H]5CN6)nc(OC[C@@]56CCCN5C[C@H](F)C6)nc4c3F)c12] | 4-(4-((1S,4S)-2,5- diazabicyclo[2.2.2] octan-2-yl)-8- fluoro-2- (((2R,7aS)-2- fluorotetrahydro- 1H-pyrrolizin- 7a(5H)- yl)methoxy) quinazolin- 7-yl)-5-ethyl-6- fluoronaphthalen- 2-ol | 604.2 (M + H)⁺ | ¹H NMR (400 MHz, METHANOL-d₄) δ ppm 8.12 (s, 2 H), 6.98 − 7.56 (m, 4 H), 5.54 − 5.72 (m, 1 H), 5.27 (br d, J = 2.1 Hz, 1 H), 4.71 − 4.80 (m, 2 H), 4.56 − 4.67 (m, 1 H), 4.41 − 4.53 (m, 1 H), 3.86 − 4.16 (m, 5 H), 3.62 − 3.73 (m, 1 H), 3.48 − 3.60 (m, 1 H), 2.13 − 2.73 (m, 12 H), 0.73 − 0.98 (m, 3 H). |
| 140 | 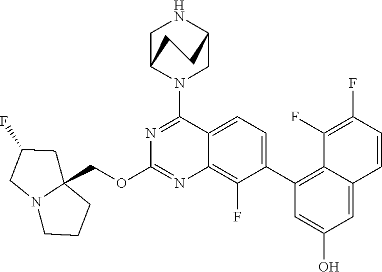 [SMILES: Oc1cc(-c2ccc3c(N4C[C@@H]5CC[C@H]4CN5)nc(OC[C@@]45CCCN4C[C@H](F)C5)nc3c2F)c2c(F)c(F)ccc2c1] | 4-(4-((1S,4S)-2,5- diazabicyclo[2.2.2] octan-2-yl)-8- fluoro-2- (((2R,7aS)-2- fluorotetrahydro- 1H-pyrrolizin- 7a(5H)-yl)methoxy) quinazolin-7- yl)-5,6- difluoronaphthalen- 2-ol | 594.2 (M + H)⁺ | ¹H NMR (400 MHz, METHANOL-d₄) δ ppm 8.13 (d, J = 9.0 Hz, 1 H), 7.65 − 7.73 (m, 1 H), 7.43 − 7.58 (m, 2 H), 7.38 (s, 1 H), 7.16 − 7.21 (m, 1 H), 5.65 − 5.71 (m, 1 H), 5.24 − 5.32 (m, 1 H), 4.78 (s, 2 H), 4.55 − 4.65 (m, 1 H), 4.42 − 4.52 (m, 1 H), 3.85 − 4.16 (m, 5 H), 3.63 − 3.74 (m, 1 H), 3.49 − 3.61 (m, 1 H), 2.13 − 2.73 (m, 10 H). |
| 141 | 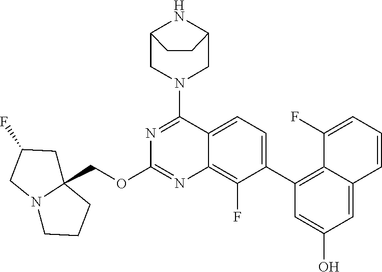 [SMILES: Oc1cc(-c2ccc3c(N4CC5CCC(C4)N5)nc(OC[C@@]45CCCN4C[C@H](F)C5)nc3c2F)c2c(F)cccc2c1] | 4-(4-((1R,5S)-3,8- diazabicyclo[3.2.1] octan-3-yl)-8- fluoro-2- (((2R,7aS)-2- fluorotetrahydro- 1H-pyrrolizin- 7a(5H)-yl)methoxy) quinazolin- 7-yl)-5- fluoronaphthalen- 2-ol | 576.1 (M + H)⁺ | ¹H NMR (400 MHz, METHANOL-d₄) δ ppm 7.98 (d, J = 8.8 Hz, 1 H), 7.66 (d, J = 7.7 Hz, 1 H), 7.41 − 7.58 (m, 2 H), 7.35 − 7.39 (m, 1 H), 7.09 − 7.13 (m, 1 H), 6.92 − 7.01 (m, 1 H), 5.53 − 5.74 (m, 1 H), 4.78 (s, 4 H), 4.33 (br s, 2 H), 3.87 − 4.18 (m, 5 H), 3.48 − 3.64 (m, 1 H), 2.74 − 2.86 (m, 1 H), 2.61 − 2.72 (m, 1 H), 2.36 − 2.56 (m, 3 H), 2.24 (s, 5 H). |
| 142 | 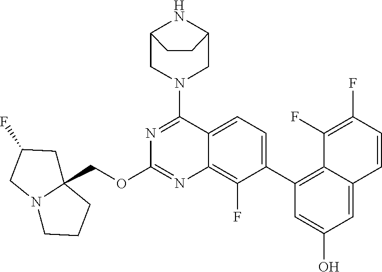 [SMILES: Oc1cc(-c2ccc3c(N4CC5CCC(C4)N5)nc(OC[C@@]45CCCN4C[C@H](F)C5)nc3c2F)c2c(F)c(F)ccc2c1] | 4-(4-((1R,5S)-3,8- diazabicyclo[3.2.1] octan-3-yl)-8- fluoro-2- (((2R,7aS)-2- fluorotetrahydro- 1H-pyrrolizin- 7a(5H)-yl)methoxy) quinazolin- 7-yl)-5,6- difluoronaphthalen- 2-ol | 594.1 (M + H)⁺ | ¹H NMR (400 MHz, METHANOL-d₄) δ ppm 7.91 − 7.97 (m, 1 H), 7.60 − 7.67 (m, 1 H), 7.37 − 7.52 (m, 2 H), 7.30 − 7.33 (m, 1 H), 7.10 − 7.17 (m, 1 H), 5.47 − 5.67 (m, 1 H), 4.68 − 4.77 (m, 4 H), 4.24 − 4.34 (m, 2 H), 3.82 − 4.11 (m, 5 H), 3.43 − 3.57 (m, 1 H), 2.70 − 2.79 (m, 1 H), 2.58 − 2.67 (m, 1 H), 2.31 − 2.53 (m, 3 H), 2.13 − 2.26 (m, 5 H). |
| 143 | 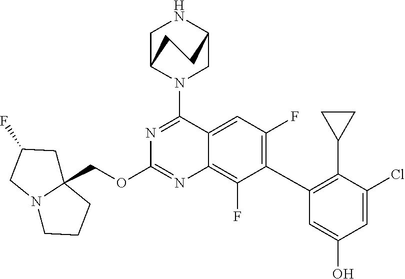 [SMILES: Oc1cc(Cl)c(C2CC2)c(-c2c(F)cc3c(N4C[C@@H]5CC[C@H]4CN5)nc(OC[C@@]45CCCN4C[C@H](F)C5)nc3c2F)c1] | 3-(4-((1R,4R)-2,5- diazabicyclo[2.2.2] octan-2-yl)-6,8- difluoro-2- (((2R,7aS)-2- fluorotetrahydro- 1H-pyrrolizin- 7a(5H)- yl)methoxy) quinazolin-7-yl)-5- chloro-4- cyclopropylphenol | 600.1 (M + H)⁺ | ¹H NMR (400 MHz, METHANOL-d₄) δ ppm 7.74 (s, 1 H), 6.93 − 7.04 (m, 1 H), 6.59 − 6.73 (m, 1 H), 5.47 − 5.67 (m, 1 H), 5.09 − 5.16 (m, 1 H), 4.63 − 4.69 (m, 2 H), 4.32 − 4.53 (m, 2 H), 3.79 − 4.09 (m, 5 H), 3.42 − 3.63 (m, 2 H), 2.03 − 2.65 (m, 10 H), 1.68 − 1.82 (m, 1 H), 0.54 − 0.69 (m, 2 H), 0.12 − 0.25 (m, 2 H). |
| 144 | 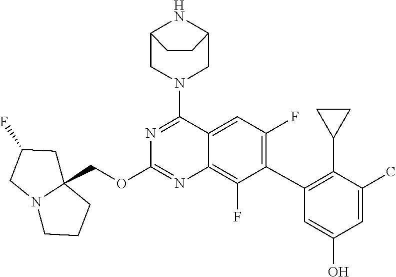 | 3-(4-((1R,5S)-3,8- diazabicyclo[3.2.1] octan-3-yl)-6,8- difluoro-2- (((2R,7aS)-2- fluorotetrahydro- 1H-pyrrolizin- 7a(5H)- yl)methoxy) quinazolin-7-yl)-5- chloro-4- cyclopropylphenol | 600.1 (M + H)⁺ | ¹H NMR (400 MHz, METHANOL-d₄) δ ppm 7.60 − 7.72 (m, 1 H), 6.93 − 7.03 (m, 1 H), 6.64 − 6.70 (m, 1 H), 5.49 − 5.67 (m, 1 H), 4.56 − 4.71 (m, 4 H), 4.18 − 4.33 (m, 2 H), 3.74 − 4.13 (m, 5 H), 3.42 − 3.58 (m, 1 H), 2.66 − 2.80 (m, 1 H), 2.53 − 2.65 (m, 1 H), 2.30 − 2.49 (m, 3 H), 2.09 − 2.27 (m, 5 H), 1.65 − 1.83 (m, 1 H), 0.53 − 0.70 (m, 2 H), 0.08 − 0.27 (m, 2 H). |
| 145 | 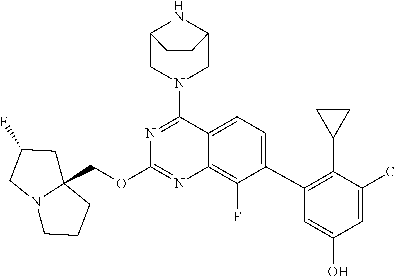 | 3-(4-((1R,5S)-3,8- diazabicyclo[3.2.1] octan-3-yl)-8- fluoro-2- (((2R,7aS)-2- fluorotetrahydro- 1H-pyrrolizin- 7a(5H)- yl)methoxy) quinazolin-7-yl)-5- chloro-4- cyclopropylphenol | 582.1 (M + H)⁺ | ¹H NMR (400 MHz, METHANOL-d₄) δ ppm 7.97 (d, J = 8.6 Hz, 1 H), 7.44 (dd, J = 8.7, 7.0 Hz, 1 H), 7.03 (d, J = 2.5 Hz, 1 H), 6.74 (d, J = 2.5 Hz, 1 H), 5.56 − 5.74 (m, 1 H), 4.75 − 4.83 (m, 4 H), 4.30 − 4.37 (m, 2 H), 3.88 − 4.17 (m, 5 H), 3.51 − 3.63 (m, 1 H), 2.62 − 2.72 (m, 1 H), 2.38 − 2.56 (m, 3 H), 2.20 − 2.31 (m, 5 H), 1.84 − 1.95 (m, 1 H), 0.53 − 0.85 (m, 2 H), 0.02 − 0.33 (m, 2 H). |
| 146 | 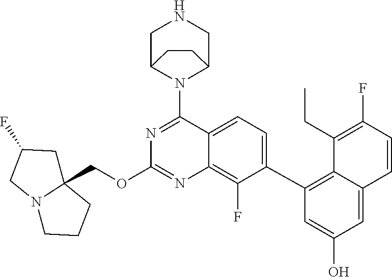 [SMILES: CCc1c(F)ccc2cc(O)cc(-c3ccc4c(N5C6CCC5CNC6)nc(OC[C@@]56CCCN5C[C@H](F)C6)nc4c3F)c12] | 4-(4-((1R,5S)-3,8- diazabicyclo[3.2.1] octan-8-yl)-8- fluoro-2- (((2R,7aS)-2- fluorotetrahydro- 1H-pyrrolizin- 7a(5H)-yl)methoxy) quinazolin- 7-yl)-5-ethyl- 6- fluoronaphthalen- 2-ol | 604.2 (M + H)⁺ | ¹H NMR (400 MHz, METHANOL-d₄) δ ppm 7.96 − 8.09 (m, 1 H), 7.63 − 7.74 (m, 1 H), 7.46 − 7.58 (m, 1 H), 7.19 − 7.36 (m, 2 H), 6.97 (d, J = 2.5 Hz, 1 H), 5.45 − 5.70 (m, 1 H), 5.21 − 5.36 (m, 2 H), 4.73 (t, J = 2.5 Hz, 2 H), 3.80 − 4.14 (m, 3 H), 3.63 − 3.77 (m, 2 H), 3.51 (s, 3 H), 2.54 − 2.83 (m, 2 H), 2.36 (br d, J = 6.1 Hz, 7 H), 2.07 − 2.26 (m, 3 H), 0.79 (t, J = 7.3 Hz, 3 H). |
| 147 | 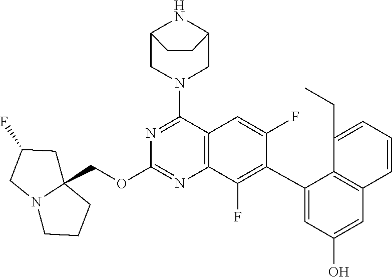 [SMILES: CCc1cccc2cc(O)cc(-c3c(F)cc4c(N5CC6CCC(C5)N6)nc(OC[C@@]56CCCN5C[C@H](F)C6)nc4c3F)c12] | 4-(4-((1R,5S)-3,8- diazabicyclo[3.2.1] octan-3-yl)-6,8- difluoro-2- (((2R,7aS)-2- fluorotetrahydro- 1H-pyrrolizin- 7a(5H)-yl)methoxy) quinazolin- 7-yl)-5- ethylnaphthalen-2- ol | 603.9 (M + H)⁺ | ¹H NMR (400 MHz, METHANOL-d₄) δ ppm 7.69 − 7.74 (m, 1 H) 7.66 (d, J = 7.52 Hz, 1 H) 7.35 − 7.42 (m, 1 H) 7.32 (d, J = 2.72 Hz, 1 H) 7.18 (d, J = 6.90 Hz, 1 H) 6.95 (d, J = 2.51 Hz, 1 H) 5.47 − 5.67 (m, 1 H) 4.70 (s, 4 H) 4.24 − 4.33 (m, 2 H) 3.79 − 4.11 (m, 5 H) 3.43 − 3.55 (m, 1 H) 2.54 − 2.81 (m, 2 H) 2.46 (br d, J = 7.32 Hz, 5 H) 2.12 − 2.29 (m, 5 H) 0.92 (td, J = 7.42, 1.46 Hz, 3 H). |
| 148 | 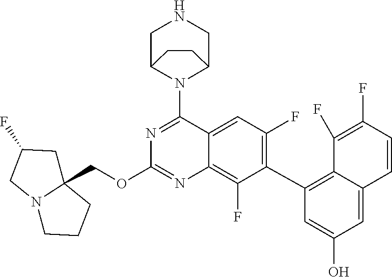 [SMILES: Oc1cc(-c2c(F)cc3c(N4C5CCC4CNC5)nc(OC[C@@]45CCCN4C[C@H](F)C5)nc3c2F)c2c(F)c(F)ccc2c1] | 4-(4-((1R,5S)-3,8- diazabicyclo[3.2.1] octan-8-yl)-6,8- difluoro-2- (((2R,7aS)-2- fluorotetrahydro- 1H-pyrrolizin- 7a(5H)-yl)methoxy) quinazolin- 7-yl)-5,6- difluoronaphthalen- 2-ol | 612.2 (M + H)⁺ | ¹H NMR (400 MHz, METHANOL-d₄) δ ppm 7.73 − 7.81 (m, 1 H) 7.62 − 7.68 (m, 1 H) 7.38 − 7.47 (m, 1 H) 7.36 (s, 1 H) 7.18 (s, 1 H) 5.51 − 5.71 (m, 1 H) 5.17 − 5.27 (m, 2 H) 4.71 (d, J = 3.76 Hz, 2 H) 3.82 − 4.09 (m, 3 H) 3.65 − 3.78 (m, 2 H) 3.48 (s, 3 H) 2.54 − 2.81 (m, 2 H) 2.28 − 2.51 (m, 5 H) 2.17 (br d, J = 8.78 Hz, 3 H). |
| 149 | 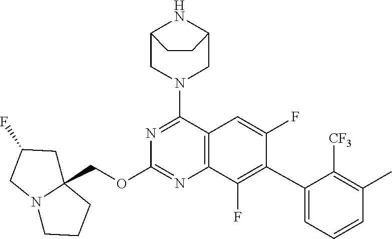 [SMILES: Cc1cccc(-c2c(F)cc3c(N4CC5CCC(C4)N5)nc(OC[C@@]45CCCN4C[C@H](F)C5)nc3c2F)c1C(F)(F)F] | 4-((1R,5S)-3,8- diazabicyclo[3.2.1] octan-3-yl)-6,8- difluoro-2- (((2R,7aS)-2- fluorotetrahydro- 1H-pyrrolizin- 7a(5H)- yl)methoxy)-7-(3- methyl-2- (trifluoromethyl) phenyl)quinazoline | 592.2 (M + H)⁺ | ¹H NMR (400 MHz, METHANOL-d₄) δ ppm 7.51 − 7.73 (m, 3 H) 7.16 − 7.33 (m, 1 H) 5.45 − 5.73 (m, 1 H) 4.69 (br s, 4 H) 4.20 − 4.33 (m, 2 H) 3.78 − 4.12 (m, 5 H) 3.43 − 3.59 (m, 1 H) 2.63 (br s, 5 H) 2.31 − 2.51 (m, 3 H) 2.17 (br s, 5 H). |
| 150 | 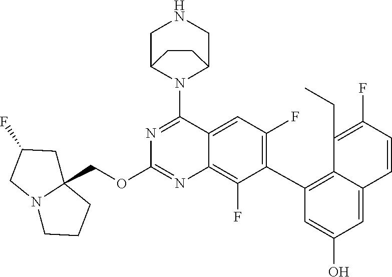 [SMILES: CCc1c(F)ccc2cc(O)cc(-c3c(F)cc4c(N5C6CCC5CNC6)nc(OC[C@@]56CCCN5C[C@H](F)C6)nc4c3F)c12] | 4-(4-((1R,5S)-3,8- diazabicyclo[3.2.1] octan-8-yl)-6,8- difluoro-2- (((2R,7aS)-2- fluorotetrahydro- 1H-pyrrolizin- 7a(5H)-yl)methoxy) quinazolin- 7-yl)-5-ethyl-6- fluoronaphthalen- 2-ol | 622.4 (M + H)⁺ | ¹H NMR (500 MHz, DMSO-d₆) δ ppm 7.78 (dd, J = 9.11, 6.05 Hz, 1 H) 7.71 (d, J = 9.17 Hz, 1 H) 7.32 − 7.40 (m, 2 H) 6.97 − 7.03 (m, 1 H) 5.18 − 5.37 (m, 1 H) 4.85 (br s, 2 H) 4.08 (br d, J = 7.46 Hz, 1 H) 4.01 (br d, J = 4.28 Hz, 1 H) 2.96 − 3.18 (m, 5 H) 2.81 (br d, J = 11.74 Hz, 3 H) 2.28 − 2.49 (m, 1 H) 2.27 − 2.53 (m, 1 H) 1.74 − 2.21 (m, 10 H) 0.75 (t, J = 7.40 Hz, 3 H). |
| 151 | 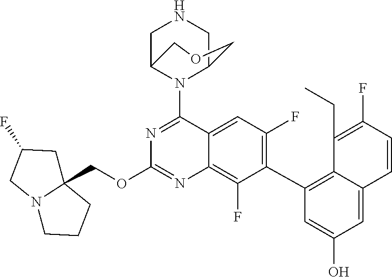 [SMILES: CCc1c(F)ccc2cc(O)cc(-c3c(F)cc4c(N5C6CNCC5COC6)nc(OC[C@@]56CCCN5C[C@H](F)C6)nc4c3F)c12] | 4-(4-((1R,5S)-3- oxa-7,9- diazabicyclo[3.3.1] nonan-9-yl)-6,8- difluoro-2- (((2R,7aS)-2- fluorotetrahydro- 1H-pyrrolizin- 7a(5H)-yl)methoxy) quinazolin- 7-yl)-5-ethyl- 6- fluoronaphthalen- 2-ol | 638.2 (M + H)⁺ | ¹H NMR (400 MHz, METHANOL-d₄) δ ppm 7.71 (dd, J = 9.20, 5.85 Hz, 1 H) 7.62 (dd, J = 9.30, 1.36 Hz, 1 H) 7.34 (d, J = 2.51 Hz, 1 H) 7.28 (t, J = 9.41 Hz, 1 H) 6.99 (d, J = 2.51 Hz, 1 H) 5.48 − 5.69 (m, 1 H) 4.82 − 4.88 (m, 2 H) 4.71 (s, 2 H) 4.21 − 4.36 (m, 4 H) 3.83 − 4.11 (m, 4 H) 3.72 − 3.81 (m, 3 H) 3.45 − 3.55 (m, 1 H) 2.53 − 2.86 (m, 3 H) 2.30 − 2.51 (m, 4 H) 2.13 − 2.29 (m, 1 H) 0.81 (td, J = 7.37, 1.57 Hz, 3 H). |
| 152 | 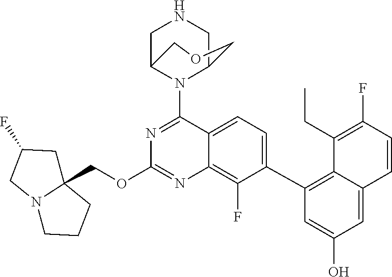 [SMILES: CCc1c(F)ccc2cc(O)cc(-c3ccc4c(N5C6CNCC5COC6)nc(OC[C@@]56CCCN5C[C@H](F)C6)nc4c3F)c12] | 4-(4-((1R,5S)-3- oxa-7,9- diazabicyclo[3.3.1] nonan-9-yl)-8- fluoro-2- (((2R,7aS)-2- fluorotetrahydro- 1H-pyrrolizin- 7a(5H)- yl)methoxy) quinazolin- 7-yl)-5-ethyl-6- fluoronaphthalen- 2-ol | 620.2 (M + H)⁺ | ¹H NMR (400 MHz, METHANOL-d₄) δ ppm 7.85 (d, J = 8.99 Hz, 1 H) 7.69 (s, 1 H) 7.45 − 7.52 (m, 1 H) 7.22 − 7.31 (m, 2 H) 6.96 (d, J = 2.72 Hz, 1 H) 5.48 − 5.67 (m, 1 H) 4.85 − 4.92 (m, 2 H) 4.72 (s, 2 H) 4.22 − 4.35 (m, 4 H) 3.74 − 4.10 (m, 7 H) 3.42 − 3.55 (m, 1 H) 2.55 − 2.87 (m, 2 H) 2.31 − 2.55 (m, 5 H) 2.10 − 2.27 (m, 1 H) 0.74 − 0.83 (m, 3 H). |
| 153 | 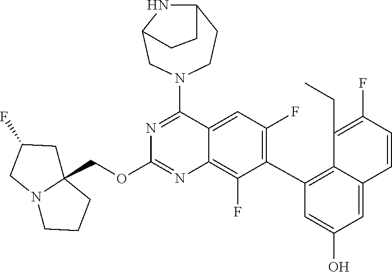 [SMILES: CCc1c(F)ccc2cc(O)cc(-c3c(F)cc4c(N5CCC6CCC(C5)N6)nc(OC[C@@]56CCCN5C[C@H](F)C6)nc4c3F)c12] | 4-(4-(3,9- diazabicyclo[4.2.1] nonan-3-yl)-6,8- difluoro-2- (((2R,7aS)-2- fluorotetrahydro- 1H-pyrrolizin- 7a(5H)-yl)methoxy) quinazolin-7- yl)-5-ethyl-6- fluoronaphthalen- 2-ol | 636.2 (M + H)⁺ | ¹H NMR (400 MHz, METHANOL-d₄) δ ppm 7.76 (br d, J = 10.16 Hz, 1 H) 7.71 (dd, J = 9.02, 5.91 Hz, 1 H) 7.33 (d, J = 2.70 Hz, 1 H) 7.24 − 7.32 (m, 1 H) 6.97 − 7.03 (m, 1 H) 5.49 − 5.69 (m, 1 H) 4.63 − 4.77 (m, 2 H) 4.50 (s, 1 H) 4.25 (br d, J = 3.94 Hz, 4 H) 3.92 (br s, 4 H) 3.44 − 3.55 (m, 1 H) 2.52 − 2.83 (m, 4 H) 2.31 − 2.51 (m, 6 H) 2.04 − 2.29 (m, 4 H) 0.78 − 0.89 (m, 3 H). |
| 154 | 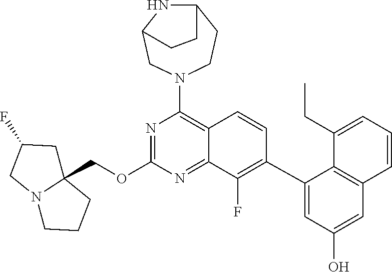 [SMILES: CCc1cccc2cc(O)cc(-c3ccc4c(N5CCC6CCC(C5)N6)nc(OC[C@@]56CCCN5C[C@H](F)C6)nc4c3F)c12] | 4-(4-(3,9- diazabicyclo[4.2.1] nonan-3-yl)-8- fluoro-2- (((2R,7aS)-2- fluorotetrahydro- 1H-pyrrolizin- 7a(5H)-yl)methoxy) quinazolin- 7-yl)-5- ethylnaphthalen-2- ol | 600.3 (M + H)⁺ | ¹H NMR (400 MHz, METHANOL-d₄) δ ppm 7.98 (d, J = 9.0 Hz, 1 H), 7.64 (d, J = 7.5 Hz, 1 H), 5.50 (d, J = 7.7 Hz, 1 H), 0.00 (t, J = 7.3 Hz, 1 H), 7.27 (d, J = 2.7 Hz, 1 H), 7.17 (dd, J = 6.7, 2.3 Hz, 1 H), 6.93 (s, 1 H), 5.65 (d, J = 51.6 Hz, 1 H), 4.60 − 4.78 (m, 4 H), 4.33 − 4.45 (m, 2 H), 3.86 − 4.25 (m, 6 H), 3.45 − 3.53 (m, 1 H), 2.01 − 2.78 (m, 14 H), 0.85 − 0.93 (m, 3 H). |
| 155 | 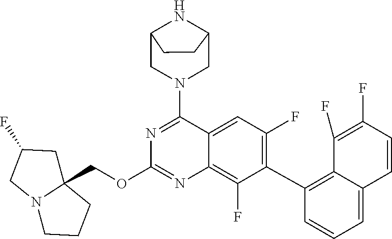 [SMILES: Fc1cc2c(N3CC4CCC(C3)N4)nc(OC[C@@]34CCCN3C[C@H](F)C4)nc2c(F)c1-c1cccc2ccc(F)c(F)c12] | 4-((1R,5S)-3,8- diazabicyclo[3.2.1] octan-3-yl)-7- (7,8- difluoronaphthalen- 1-yl)-6,8- difluoro-2- (((2R,7aS)-2- fluorotetrahydro- 1H-pyrrolizin- 7a(5H)- yl)methoxy) quinazoline | 595.9 (M + H)⁺ | ¹H NMR (400 MHz, METHANOL-d₄) δ ppm 8.15 (d, J = 8.15 Hz, 1 H) 7.92 (ddd, J = 6.95, 4.86, 2.61 Hz, 1 H) 7.67 − 7.75 (m, 2 H) 7.61 (d, J = 6.90 Hz, 2 H) 5.44 − 5.71 (m, 1 H) 4.65 − 4.76 (m, 4 H) 4.23 − 4.33 (m, 2 H) 3.78 − 4.11 − (m, 5 H) 3.44 − 3.56 (m, 1 H) 2.53 2.79 (m, 2 H) 2.32 − 2.50 (m, 3 H) 2.11 − 2.28 (m, 5 H). |
| 156 | 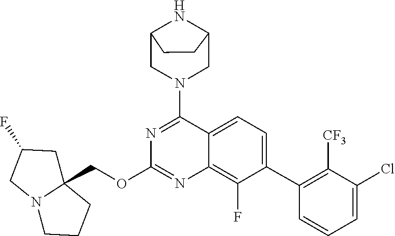 [SMILES: Fc1c(-c2cccc(Cl)c2C(F)(F)F)ccc2c(N3CC4CCC(C3)N4)nc(OC[C@@]34CCCN3C[C@H](F)C4)nc12] | 4-((1R,5S)-3,8- diazabicyclo[3.2.1] octan-3-yl)-7-(3- chloro-2- (trifluoromethyl)p henyl)-8-fluoro-2- (((2R,7aS)-2- fluorotetrahydro- 1H-pyrrolizin- 7a(5H)- yl)methoxy) quinazoline | 594.2 (M + H)⁺ | ¹H NMR (400 MHz, METHANOL-d₄) δ ppm 7.89 − 7.95 (m, 1 H) 7.77 − 7.82 (m, 1 H) 7.68 − 7.74 (m, 1 H) 7.30 − 7.39 (m, 2 H) 5.41 − 5.71 (m, 1 H) 4.66 − 4.72 (m, 3 H) 4.21 − 4.30 (m, 2 H) 3.83 − 4.09 (m, 5 H) 3.45 − 3.57 (m, 1 H) 2.55 − 2.83 (m, 2 H) 2.29 − 2.52 (m, 3 H) 2.12 − 2.26 (m, 5 H) 1.36 − 1.42 (m, 1 H). |
| 157 | 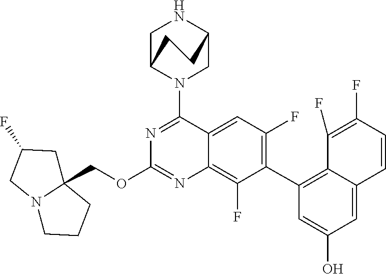 [SMILES: Oc1cc(-c2c(F)cc3c(N4C[C@@H]5CC[C@H]4CN5)nc(OC[C@@]45CCCN4C[C@H](F)C5)nc3c2F)c2c(F)c(F)ccc2c1] | 4-(4-((1S,4S)-2,5- diazabicyclo[2.2.2] octan-2-yl)-6,8- difluoro-2- (((2R,7aS)-2- fluorotetrahydro- 1H-pyrrolizin- 7a(5H)-yl)methoxy) quinazolin- 7-yl)-5,6- difluoronaphthalen- 2-ol | 612.2 (M + H)⁺ | ¹H NMR (400 MHz, METHANOL-d₄) δ ppm 7.79 − 7.87 (m, 1 H), 7.61 − 7.68 (m, 1 H), 7.36 (t, J = 2.1 Hz, 2 H), 7.17 (d, J = 2.1 Hz, 1 H), 5.43 − 5.67 (m, 1 H), 5.11 − 5.19 (m, 1 H), 4.61 − 4.70 (m, 2 H), 4.46 − 4.56 (m, 1 H), 4.35 − 4.43 (m, 1 H), 3.97 (br s, 2 H), 3.79 − 3.91 (m, 3 H), 3.56 − 3.64 (m, 1 H), 3.41 − 3.52 (m, 1 H), 2.57 − 2.77 (m, 2 H), 2.40 − 2.56 (m, 2 H), 2.31 − 2.40 (m, 2 H), 2.19 − 2.29 (m, 1 H), 2.05 − 2.17 (m, 3 H). |
| 158 | 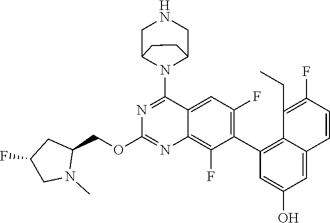 [SMILES: CCc1c(F)ccc2cc(O)cc(-c3c(F)cc4c(N5C6CCC5CNC6)nc(OC[C@@H]5C[C@@H](F)CN5C)nc4c3F)c12] | 4-(4-((1R,5S)-3,8- diazabicyclo[3.2.1] octan-8-yl)-6,8- difluoro-2- (((2S,4R)-4- fluoro-1- methylpyrrolidin- 2-yl)methoxy) quinazolin- 7-yl)-5-ethyl- 6- fluoronaphthalen- 2-ol | 596.4 (M + H)⁺ | ¹H NMR (400 MHz, METHANOL-d₄) δ ppm 7.69 (d, J = 9.0 Hz, 2 H), 7.31 (d, J = 2.7 Hz, 1 H), 7.26 (s, 1 H), 6.99 − 7.03 (m, 1 H), 5.11 − 5.32 (m, 1 H), 4.97 − 5.04 (m, 2 H), 4.53 (s, 2 H), 3.48 − 3.63 (m, 1 H), 3.35 − 3.41 (m, 2 H), 3.18 − 3.27 (m, 1 H), 2.98 − 3.06 (m, 2 H), 2.68 − 2.80 (m, 1 H), 2.61 (s, 4 H), 2.41 − 2.50 (m, 1 H), 2.27 − 2.39 (m, 1 H), 1.94 − 2.24 (m, 5 H), 0.83 (s, 3 H). |
| 159 | 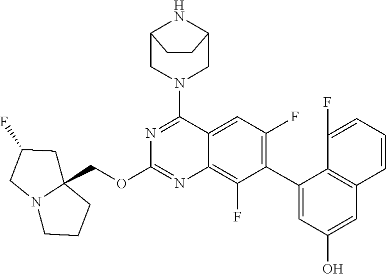 [SMILES: Oc1cc(-c2c(F)cc3c(N4CC5CCC(C4)N5)nc(OC[C@@]45CCCN4C[C@H](F)C5)nc3c2F)c2c(F)cccc2c1] | 4-(4-((1R,5S)-3,8- diazabicyclo[3.2.1] octan-3-yl)-6,8- difluoro-2- (((2R,7aS)-2- fluorotetrahydro- 1H-pyrrolizin- 7a(5H)- yl)methoxy) quinazolin- 7-yl)-5- fluoronaphthalen- 2-ol | 594.2 (M + H)⁺ | ¹H NMR (400 MHz, METHANOL-d₄) δ ppm 7.65 − 7.70 (m, 1 H) 7.59 − 7.64 (m, 1 H) 7.38 − 7.46 (m, 1 H) 7.36 (s, 1 H) 7.10 (dd, J = 2.30, 1.05 Hz, 1 H) 6.88 − 6.98 (m, 1 H) 5.51 − 5.67 (m, 1 H) 4.63 − 4.76 (m, 4 H) 4.25 − 4.33 (m, 2 H) 3.79 − 4.10 (m, 5 H) 3.44 − 3.55 (m, 1 H) 2.55 − 2.80 (m, 2 H) 2.32 − 2.51 (m, 3 H) 2.19 (br s, 5 H). |
| 160 | 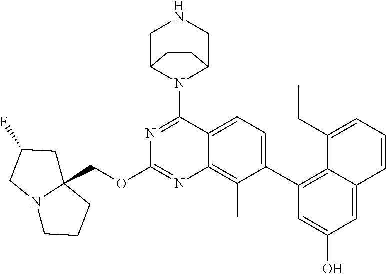 [SMILES: CCc1cccc2cc(O)cc(-c3ccc4c(N5C6CCC5CNC6)nc(OC[C@@]56CCCN5C[C@H](F)C6)nc4c3C)c12] | 4-(4-((1R,5S)-3,8- diazabicyclo[3.2.1] octan-8-yl)-2- (((2R,7aS)-2- fluorotetrahydro- 1H-pyrrolizin- 7a(5H)- yl)methoxy)-8- methylquinazolin- 7-yl)-5- ethylnaphthalen-2- ol | 582.2 (M + H)⁺ | ¹H NMR (400 MHz, METHANOL-d₄) δ ppm 8.00 (d, J = 8.8 Hz, 1 H), 7.63 (d, J = 7.7 Hz, 1 H), 7.45 (d, J = 8.6 Hz, 1 H), 7.36 (t, J = 7.6 Hz, 1 H), 7.23 (d, J = 2.5 Hz, 1 H), 7.13 (d, J = 6.9 Hz, 1 H), 6.79 (d, J = 2.7 Hz, 1 H), 0.00 (dd, J = 52.0, 3.8 Hz, 1 H), 5.16 (br s, 2 H), 4.70 − 4.74 (m, 2 H), 3.90 − 4.07 (m, 3 H), 3.69 − 3.76 (m, 2 H), 3.45 − 3.56 (m, 3 H), 2.30 (s, 17 H), 0.83 (t, J = 7.3 Hz, 3 H). |
| 161 | 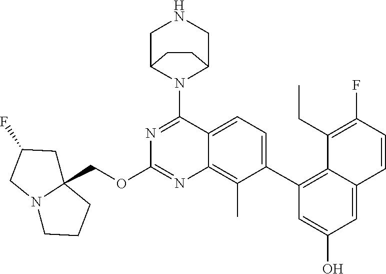 [SMILES: CCc1c(F)ccc2cc(O)cc(-c3ccc4c(N5C6CCC5CNC6)nc(OC[C@@]56CCCN5C[C@H](F)C6)nc4c3C)c12] | 4-(4-((1R,5S)-3,8- diazabicyclo[3.2.1] octan-8-yl)-2- (((2R,7aS)-2- fluorotetrahydro- 1H-pyrrolizin- 7a(5H)- yl)methoxy)-8- methylquinazolin- 7-yl)-5-ethyl-6- fluoronaphthalen- 2-ol | 600.2 (M + H)⁺ | ¹H NMR (400 MHz, METHANOL-d₄) δ ppm 8.39 (dd, J = 9.5, 6.0 Hz, 1 H), 0.00 (d, J = 8.6 Hz, 1 H), 7.46 − 7.52 (m, 1 H), 0.00 (t, J = 10.0 Hz, 1 H), 7.00 (s, 1 H), 5.53 − 5.73 (m, 1 H), 5.17 (br s, 2 H), 4.99 (s, 1 H), 4.69 − 4.76 (m, 2 H), 3.88 − 4.10 (m, 3 H), 3.67 − 3.76 (m, 2 H), 3.44 − 3.55 (m, 3 H), 2.28 (br d, J = 6.7 Hz, 16 H), 0.63 − 0.77 (m, 3 H). |
| 162 | 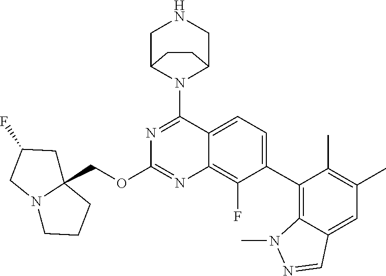 [SMILES: Cc1cc2cnn(C)c2c(-c2ccc3c(N4C5CCC4CNC5)nc(OC[C@@]45CCCN4C[C@H](F)C5)nc3c2F)c1C] | 4-((1R,5S)-3,8- diazabicyclo[3.2.1] octan-3-yl)-8- fluoro-2- (((2R,7aS)-2- fluorotetrahydro- 1H-pyrrolizin- 7a(5H)- yl)methoxy)-7- (1,5,6-trimethyl- 1H-indazol-7- yl)quinazoline | 573.8 (M + H)⁺ | ¹H NMR (400 MHz, METHANOL-d₄) δ ppm 7.95 (s, 2 H), 7.67 (s, 1 H), 7.12 − 7.50 (m, 1 H), 5.44 − 5.75 (m, 1 H), 4.73-4.81 (m, 4 H), 4.21 − 4.42 (m, 2 H), 3.78 − 4.11 (m, 5 H), 3.46 − 3.57 (m, 1 H), 3.42 (s, 3 H), 2.55 − 2.83 (m, 2 H), 2.46-2.50 (m, 6 H), 2.14- 2.40 (m, δ H). |
| 163 | 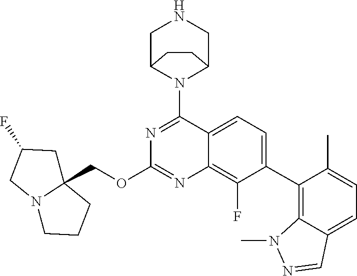 [SMILES: Cc1ccc2cnn(C)c2c1-c1ccc2c(N3C4CCC3CNC4)nc(OC[C@@]34CCCN3C[C@H](F)C4)nc2c1F] | 4-((1R,5S)-3,8- diazabicyclo[3.2.1] octan-3-yl)-7- (1,6-dimethyl-1H- indazol-7-yl)-8- fluoro-2- (((2R,7aS)-2- fluorotetrahydro- 1H-pyrrolizin- 7a(5H)- yl)methoxy) quinazoline | 560.2 (M + H)⁺ | ¹H NMR (400 MHz, METHANOL-d₄) δ ppm 8.03 (s, 2 H), 7.68 − 7.80 (m, 1 H), 7.34 − 7.48 (m, 1 H), 7.06 − 7.26 (m, 1 H), 5.44 − 5.91 (m, 1 H), 4.73-4.81 (m, 4 H), 4.16 − 4.40 (m, 2 H), 3.83 − 4.12 (m,5 H), 3.48-3.51 (m, 4 H), 2.54 − 2.84 (m, 2 H), 2.27 − 2.51 (m, 3 H), 2.20-2.24 (m, 8 H). |
| 164 | 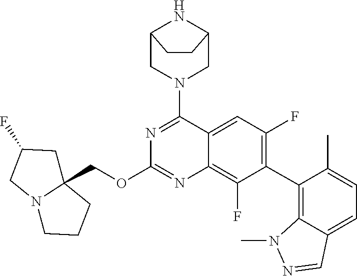 [SMILES: Cc1ccc2cnn(C)c2c1-c1c(F)cc2c(N3CC4CCC(C3)N4)nc(OC[C@@]34CCCN3C[C@H](F)C4)nc2c1F] | 4-((1R,5S)-3,8- diazabicyclo[3.2.1] octan-3-yl)-7- (1,6-dimethyl-1H- indazol-7-yl)-6,8- difluoro-2- (((2R,7aS)-2- fluorotetrahydro- 1H-pyrrolizin- 7a(5H)- yl)methoxy) quinazoline | 578.2 (M + H)⁺ | ¹H NMR (400 MHz, METHANOL-d₄) δ ppm 8.05 (s, 1 H), 7.69 − 7.91 (m, 2 H), 7.01 − 7.34 (m, 1 H), 5.46 − 5.74 (m, 1 H), 4.61-4.81 (m, 4 H), 4.21 − 4.38 (m, 2 H), 3.84 − 4.10 (m, 5 H), 3.55 (s, 4 H), 2.55 − 2.86 (m, 2 H), 2.31 − 2.52 (m, 3 H), 2.27 (s, 8 H). |
| 165 | 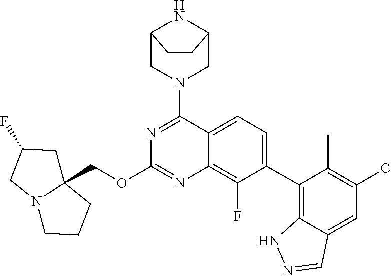 | 4-((1R,5S)-3,8- diazabicyclo[3.2.1] octan-3-yl)-7-(5- chloro-6-methyl- 1H-indazol-7-yl)- 8-fluoro-2- (((2R,7aS)-2- fluorotetrahydro- 1H-pyrrolizin- 7a(5H)- yl)methoxy) quinazoline | 580.2 (M + H)⁺ | ¹H NMR (400 MHz, METHANOL-d₄) δ ppm 8.03 − 8.16 (m, 1 H), 7.98 (s, 1 H), 7.90 − 8.00 (m, 1 H), 7.34 − 7.49 (m, 1 H), 5.36 − 5.82 (m, 1 H), 4.71 − 4.81 (m, 4 H), 4.23 − 4.40 (m, 2 H), 3.85 − 4.04 (m, 5 H), 3.44 − 3.62 (m, 1 H), 2.56 − 2.82 (m, 2 H), 2.33-2.50 (m, 6 H), 2.19 (br s, 5 H). |
| 166 | 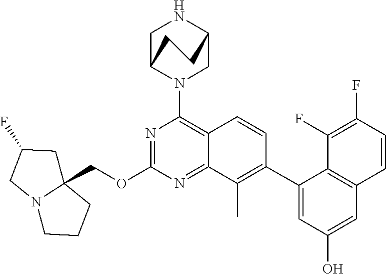 [SMILES: Cc1c(-c2cc(O)cc3ccc(F)c(F)c23)ccc2c(N3C[C@@H]4CC[C@H]3CN4)nc(OC[C@@]34CCCN3C[C@H](F)C4)nc12] | 4-(4-((1S,4S)-2,5- diazabicyclo[2.2.2] octan-2-yl)-2- (((2R,7aS)-2- fluorotetrahydro- 1H-pyrrolizin- 7a(5H)- yl)methoxy)-8- methylquinazolin- 7-yl)-5,6- difluoronaphthalen- 2-ol | 590.2 (M + H)⁺ | ¹H NMR (400 MHz, METHANOL-d₄) δ ppm 8.24 − 8.32 (m, 1 H), 8.04 (d, J = 8.6 Hz, 1 H), 7.37 (dt, J = 8.0, 4.0 Hz, 1 H), 7.24 (q, J = 8.8 Hz, 1 H), 7.13 (d, J = 2.1 Hz, 1 H), 5.62 (td, J = 51.6, 3.8 Hz, 1 H), 5.14 (br s, 1 H), 4.97 − 5.01 (m, 1 H), 4.72 − 4.76 (m, 2 H), 4.32 − 4.57 (m, 2 H), 3.85 − 4.11 (m, 5 H), 3.47 − 3.65 (m, 2 H), 2.30 (d, J = 6.7 Hz, 14 H). |
| 168 | 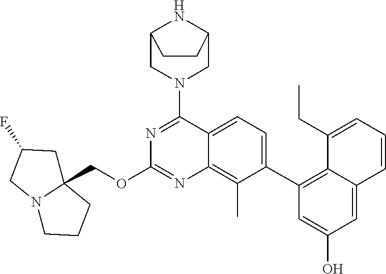 [SMILES: CCc1cccc2cc(O)cc(-c3ccc4c(N5CC6CCC(C5)N6)nc(OC[C@@]56CCCN5C[C@H](F)C6)nc4c3C)c12] | 4-(4-((1R,5S)-3,8- diazabicyclo[3.2.1] octan-3-yl)-2- (((2R,7aS)-2- fluorotetrahydro- 1H-pyrrolizin- 7a(5H)- yl)methoxy)-8- methylquinazolin- 7-yl)-5- ethylnaphthalen-2- ol | 582.4 (M + H)⁺ | ¹H NMR (400 MHz, METHANOL-d₄) δ ppm 7.92 (br d, J = 8.4 Hz, 1 H), 7.63 (br d, J = 7.9 Hz, 1 H), 7.32 − 7.46 (m, 2 H), 7.23 (br s, 1 H), 7.13 (br d, J = 6.7 Hz, 1 H), 6.78 (br s, 1 H), 5.51 − 5.71 (m, 1 H), 4.58 (br d, J = 14.2 Hz, 3 H), 4.27 (br s, 2 H), 3.88 − 4.08 (m, 3 H), 3.78 (br t, J = 11.9 Hz, 2 H), 3.50 (br d, J = 4.2 Hz, 1 H), 2.13 − 2.86 (m, 16 H), 0.83 (br s, 3 H). |
| 169 | 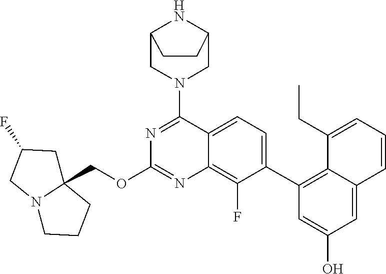 [SMILES: CCc1cccc2cc(O)cc(-c3ccc4c(N5CC6CCC(C5)N6)nc(OC[C@@]56CCCN5C[C@H](F)C6)nc4c3F)c12] | 4-(4-((1R,5S)-3,8- diazabicyclo[3.2.1] octan-3-yl)-8- fluoro-2- (((2R,7aS)-2- fluorotetrahydro- 1H-pyrrolizin- 7a(5H)- yl)methoxy)quinaz olin-7-yl)-5- ethylnaphthalen-2- ol | 586.2 (M + H)⁺ | ¹H NMR (400 MHz, METHANOL-d₄) δ ppm 7.92 (br d, J = 8.8 Hz, 1 H), 7.64 (br d, J = 7.9 Hz, 1 H), 7.44 (br d, J = 8.4 Hz, 1 H), 7.30 − 7.40 (m, 1 H), 7.24 − 7.30 (m, 1 H), 7.12 − 7.19 (m, 1 H), 6.86 − 6.96 (m, 1 H), 5.47 − 5.69 (m, 1 H), 4.24 − 4.32 (m, 2 H), 3.78 − 4.00 (m, 5 H), 3.42 − 3.57 (m, 1 H), 2.54 − 2.71 (m, 5 H), 2.29 − 2.51 (m, 6 H), 2.13 − 2.29 (m, 5 H), 0.88 (br t, J = 6.5 Hz, 3 H). |
| 170 | 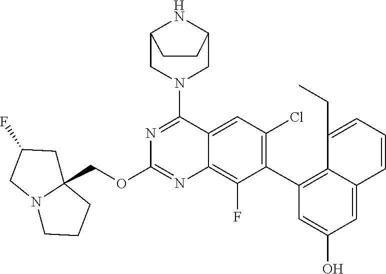 | 4-(4-((1R,5S)-3,8- diazabicyclo[3.2.1] octan-3-yl)-6- chloro-8-fluoro-2- (((2R,7aS)-2- fluorotetrahydro- 1H-pyrrolizin- 7a(5H)- yl)methoxy) quinazolin- 7-yl)-5- ethylnaphthalen-2- ol | 586.2 (M + H)⁺ | ¹H NMR (400 MHz, METHANOL-d₄) δ ppm 8.00 (d, J = 1.3 Hz, 1 H), 7.65 (d, J = 7.7 Hz, 1 H), 7.38 (t, J = 7.7 Hz, 1 H), 7.30 (d, J = 2.7 Hz, 1 H), 7.16 (d, J = 7.1 Hz, 1 H), 6.85 (d, J = 2.7 Hz, 1 H), 5.48 − 5.68 (m, 1 H), 4.64 − 4.70 (m, 2 H), 4.28 (br d, J = 8.8 Hz, 2 H), 3.82 − 4.07 (m, 6 H), 3.43 − 3.55 (m, 1 H), 2.54 − 2.65 (m, 1 H), 2.32 − 2.51 (m, 6 H), 2.12 − 2.25 (m, 6 H), 0.94 (td, J = 7.4, 1.9 Hz, 3 H). |
| 171 | 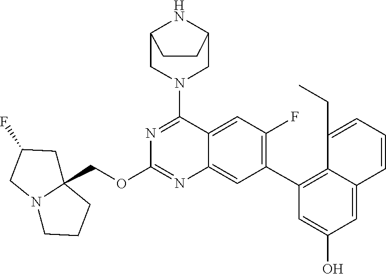 [SMILES: CCc1cccc2cc(O)cc(-c3cc4nc(OC[C@@]56CCCN5C[C@H](F)C6)nc(N5CC6CCC(C5)N6)c4cc3F)c12] | 4-(4-((1R,5S)-3,8- diazabicyclo[3.2.1] octan-3-yl)-6- fluoro-2- (((2R,7aS)-2- fluorotetrahydro- 1H-pyrrolizin- 7a(5H)- yl)methoxy) quinazolin-7-yl)-5- ethylnaphthalen-2- ol | 586.2 (M + H)⁺ | ¹H NMR (400 MHz, METHANOL-d₄) δ ppm 7.79 (s, 1 H), 7.75 (s, 1 H), 7.58 − 7.66 (m, 1 H), 7.37 (t, J = 1.0 Hz, 1 H), 7.28 (br s, 1 H), 7.17 (br d, J = 6.7 Hz, 1 H), 6.87 − 6.94 (m, 1 H), 5.49 − 5.73 (m, 1 H), 4.22 − 4.33 (m, 2 H), 3.72 − 4.06 (m, 6 H), 3.45 − 3.57 (m, 1 H), 2.56 − 2.86 (m, 3 H), 2.31 − 2.51 (m, 6 H), 2.12 − 2.31 (m, 6 H), 0.89 (br t, J = 7.1 Hz, 3 H). |
| 172 | 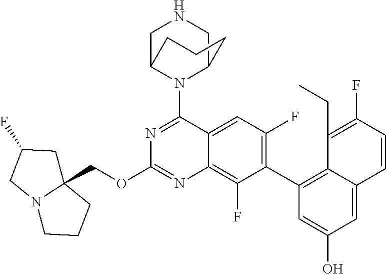 [SMILES: CCc1c(F)ccc2cc(O)cc(-c3c(F)cc4c(N5C6CCCC5CNC6)nc(OC[C@@]56CCCN5C[C@H](F)C6)nc4c3F)c12] | 4-(4-((1R,5S)-3,9- diazabicyclo[3.3.1] nonan-9-yl)-6,8- difluoro-2- (((2R,7aS)-2- fluorotetrahydro- 1H-pyrrolizin- 7a(5H)-yl)methoxy) quinazolin- 7-yl)-5-ethyl- 6- fluoronaphthalen- 2-ol | 636.3 (M + H)⁺ | ¹H NMR (METHANOL-d₄, 400 MHz) δ 7.7-7.8 (m, 2 H), 7.36 (s, 1 H), 7.29 (t, 1 H, J = 9.4 Hz), 7.04 (br s, 1 H), 5.5-5.7 (m, 1 H), 5.27 (br s, 2 H), 3.8-4.1 (m, 4 H), 3.5- 3.8 (m, 6 H), 3.4-3.5 (m, 1 H), 2.1- 2.7 (m, 14 H), 0.87 (br s, 3 H). |
| 173 | 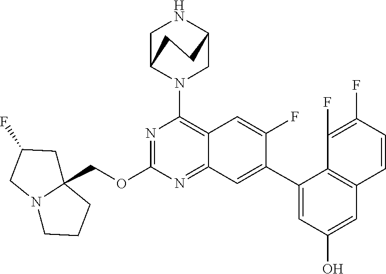 [SMILES: Oc1cc(-c2cc3nc(OC[C@@]45CCCN4C[C@H](F)C5)nc(N4C[C@@H]5CC[C@H]4CN5)c3cc2F)c2c(F)c(F)ccc2c1] | 4-(4-((1S,4S)-2,5- diazabicyclo[2.2.2] octan-2-yl)-6- fluoro-2- (((2R,7aS)-2- fluorotetrahydro- 1H-pyrrolizin- 7a(5H)-yl)methoxy) quinazolin- 7-yl)-5,6- difluoronaphthalen- 2-ol | 594.1 (M + H)⁺ | ¹H NMR (400 MHz, DMSO-d₆) δ ppm 7.88 (d, J = 11.50 Hz, 1 H) 7.68 − 7.78 (m, 1 H) 7.56 (d, J = 7.52 Hz, 2 H) 7.36 (t, J = 2.09 Hz, 1 H) 7.12 (s, 1 H) 5.15 − 5.45 (m, 1 H) 4.72 (br s, 1 H) 4.13 − 4.22 (m, 1 H) 4.04 − 4.12 (m, 2 H) 3.94 − 4.03 (m, 1 H) 2.97 − 3.18 (m, 6 H) 2.76 − 2.90 (m, 1 H) 1.66 − 2.20 (m, 10 H). |
| 174 | 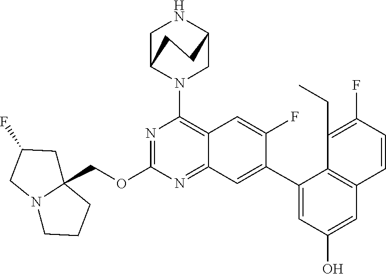 [SMILES: CCc1c(F)ccc2cc(O)cc(-c3cc4nc(OC[C@@]56CCCN5C[C@H](F)C6)nc(N5C[C@@H]6CC[C@H]5CN6)c4cc3F)c12] | 4-(4-((1S,4S)-2,5- diazabicyclo[2.2.2] octan-2-yl)-6- fluoro-2- (((2R,7aS)-2- fluorotetrahydro- 1H-pyrrolizin- 7a(5H)- yl)methoxy) quinazolin-7-yl)- 5-ethyl-6- fluoronaphthalen- 2-ol | 604.2 (M + H)⁺ | ¹H NMR (400 MHz, DMSO-d₆) δ ppm 10.94 − 11.07 (br s, 1 H) 9.91 − 10.07 (br s, 1 H) 9.30 (br s, 2 H) 7.92 (br d, J = 10.87 Hz, 1 H) 7.77 (dd, J = 8.99, 6.06 Hz, 1 H) 7.71 (dd, J = 7.42, 1.78 Hz, 1 H) 7.30 − 7.41 (m, 2 H) 6.93 (d, J = 2.51 Hz, 1 H) 5.48 − 5.68 (m, 1 H) 4.97 (br s, 1 H) 4.58 (s, 2 H) 4.23 − 4.42 (m, 3 H) 3.68 − 3.94 (m, 5 H) 3.56 − 3.66 (m, 1 H) 3.39 − 3.50 (m, 1 H) 3.25 − 3.36 (m, 1 H) 2.53 − 2.63 (m, 1 H) 1.87 − 2.36 (m, 9 H) 0.76 (t, J = 7.32 Hz, 3 H). |
| 175 | 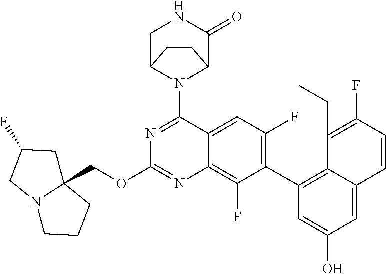 [SMILES: CCc1c(F)ccc2cc(O)cc(-c3c(F)cc4c(N5C6CCC5C(=O)NC6)nc(OC[C@@]56CCCN5C[C@H](F)C6)nc4c3F)c12] | 8-(7-(8-ethyl-7- fluoro-3- (methoxymethoxy) naphthalen-1-yl)- 6,8-difluoro-2- (((2R,7aS)-2- fluorotetrahydro- 1H-pyrrolizin- 7a(5H)-yl)methoxy) quinazolin- 4-yl)-3,8- diazabicyclo[3.2.1] octan-2-one Isomer 1 | 636.1 (M + H)⁺ | ¹H NMR (400 MHz, METHANOL-d₄) δ ppm 7.91 (dd, J = 9.4, 1.7 Hz, 1 H), 7.71 (dd, J = 9.0, 5.9 Hz, 1 H), 7.34 (d, J = 2.5 Hz, 1 H), 7.28 (t, J = 9.4 Hz, 1 H), 7.01 (d, J = 2.7 Hz, 1 H), 5.50 − 5.70 (m, 1 H), 5.24 − 5.32 (m, 1 H), 4.68 (s, 2 H), 3.87 − 4.10 (m, 3 H), 3.36 − 3.54 (m, 3 H), 3.24 (dd, J = 12.3, 1.5 Hz, 1 H), 2.14 − 2.81 (m, 14 H), 0.83 (br d, J = 1.7 Hz, 3 H). |
| 176 | 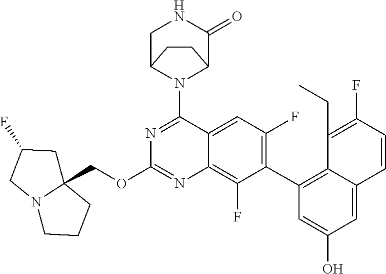 [SMILES: CCc1c(F)ccc2cc(O)cc(-c3c(F)cc4c(N5C6CCC5C(=O)NC6)nc(OC[C@@]56CCCN5C[C@H](F)C6)nc4c3F)c12] | 8-(7-(8-ethyl-7- fluoro-3- (methoxymethoxy) naphthalen-1-yl)- 6,8-difluoro-2- (((2R,7aS)-2- fluorotetrahydro- 1H-pyrrolizin- 7a(5H)-yl)methoxy) quinazolin-4- yl)-3,8- diazabicyclo[3.2.1 octan-2-one Isomer 2 | 636.1 (M + H)⁺ | ¹H NMR (400 MHz, METHANOL-d₄) δ ppm 7.93 (d, J = 8.1 Hz, 1 H), 7.71 (dd, J = 9.1, 6.0 Hz, 1 H), 0.00 (d, J = 2.5 Hz, 1 H), 0.00 (t, J = 9.5 Hz, 1 H), 7.03 (d, J = 2.5 Hz, 1 H), 5.51 − 5.68 (m, 1 H), 5.25 − 5.31 (m, 1 H), 4.60 − 4.73 (m, 2 H), 3.86 − 4.13 (m, 3 H), 3.38 − 3.54 (m, 2 H), 3.15 − 3.26 (m, 1 H), 2.12 − 2.79 (m, 13 H), 0.80 (td, J = 7.4, 2.5 Hz, 3 H). |
| 178 | 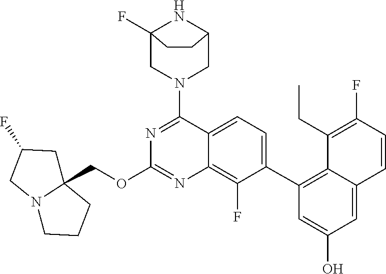 [SMILES: CCc1c(F)ccc2cc(O)cc(-c3ccc4c(N5CC6CCC(F)(C5)N6)nc(OC[C@@]56CCCN5C[C@H](F)C6)nc4c3F)c12] | 5-ethyl-6-fluoro-4- (8-fluoro-4- ((1S,5S)-1-fluoro- 3,8- diazabicyclo[3.2.1] octan-3-yl)-2- (((2R,7aS)-2- fluorotetrahydro- 1H-pyrrolizin- 7a(5H)- yl)methoxy) quinazolin-7- yl)naphthalen-2-ol | 622.0 (M + H)⁺ | ¹H NMR (400 MHz, METHANOL-d₄) δ ppm 7.97 (d, J = 8.78 Hz, 1 H) 7.69 (dd, J = 8.99, 5.85 Hz, 1 H) 7.49 (s, 1 H) 7.22 − 7.31 (m, 2 H) 6.97 (s, 1 H) 5.48 − 5.72 (m, 1 H) 4.97 − 5.12 (m, 1 H) 4.71 − 4.74 (m, 2 H) 4.59 − 4.70 (m, 1 H) 4.19 − 4.27 (m, 1 H) 3.74 − 4.11 (m, 5 H) 3.44 − 3.57 (m, 1 H) 2.55 − 2.82 (m, 2 H) 2.31 − 2.55 (m, 7 H) 2.13 − 2.28 (m, 2 H) 1.86 − 2.01 (m, 1 H) 0.76 − 0.82 (m, 3 H). |
| 179 | 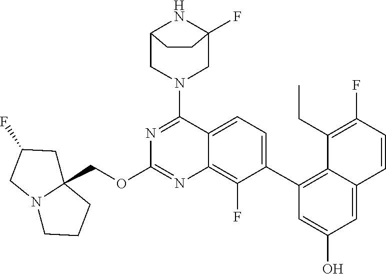 [SMILES: CCc1c(F)ccc2cc(O)cc(-c3ccc4c(N5CC6CCC(F)(C5)N6)nc(OC[C@@]56CCCN5C[C@H](F)C6)nc4c3F)c12] | 5-ethyl-6-fluoro-4- (8-fluoro-4- ((1R,5R)-1-fluoro- 3,8- diazabicyclo[3.2.1] octan-3-yl)-2- (((2R,7aS)-2- fluorotetrahydro- 1H-pyrrolizin- 7a(5H)- yl)methoxy) quinazolin-7- yl)naphthalen-2-ol | 622.2 (M + H)⁺ | ¹H NMR (400 MHz, METHANOL-d₄) δ ppm 7.93 − 8.00 (m, 1 H) 7.65 − 7.72 (m, 1 H) 7.45 − 7.52 (m, 1 H) 7.23 − 7.31 (m, 2 H) 6.97 (d, J = 2.51 Hz, 1 H) 5.47 − 5.68 (m, 1 H) 4.94 − 5.05 (m, 1 H) 4.69 − 4.74 (m, 2 H) 4.59 − 4.69 (m, 1 H) 4.10 − 4.23 (m, 1 H) 3.69 − 4.09 (m, 5 H) 3.42 − 3.55 (m, 1 H) 2.53 − 2.83 (m, 2 H) 2.32 − 2.53 (m, 7 H) 2.06 − 2.27 (m, 2 H) 1.86 − 1.99 (m, 1 H) 0.79 (s, 3 H). |

### 4-(4-((1S,4S)-2,5-diazabicyclo[2.2.2]octan-2-yl)-6,8-difluoro-2-(((2R,7aS)-2-fluorotetrahydro-1H-pyrrolizin-7a(5H)-yl)methoxy)quinazolin-7-yl)-5-ethynylnaphthalen-2-ol (Example 180

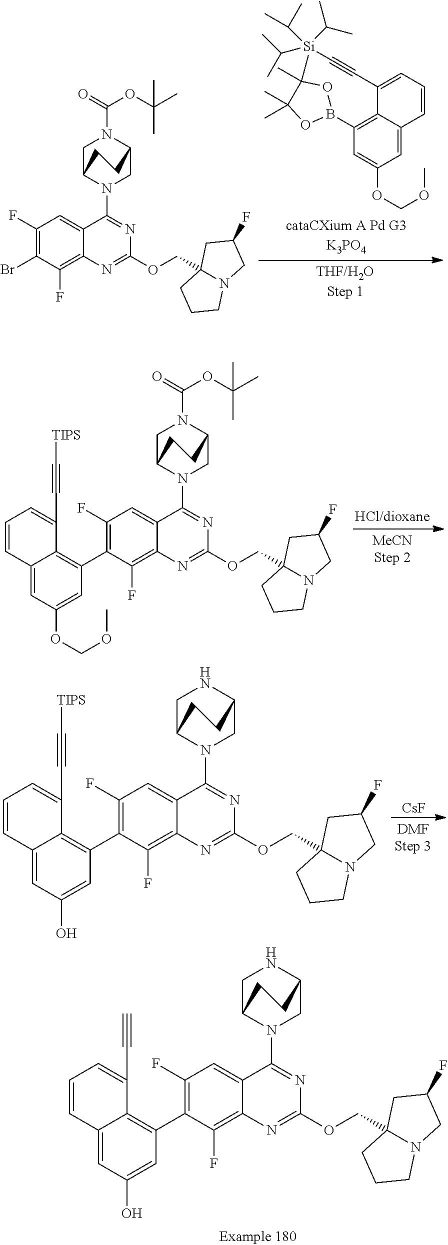

Step 1: tert-Butyl (1S,4S)-5-(6,8-difluoro-2-(((2R,7aS)-2-fluorotetrahydro-1H-pyrrolizin-7a(5H)-yl)methoxy)-7-(3-(methoxymethoxy)-8-((triisopropylsilyl)ethynyl)naphthalen-1-yl)quinazolin-4-yl)-2,5-diazabicyclo[2.2.2]octane-2-carboxylate. A vial was charged with tert-butyl (1S,4S)-5-(7-bromo-6,8-difluoro-2-(((2R,7aS)-2-fluorotetrahydro-1H-pyrrolizin-7a(5H)-yl)methoxy)quinazolin-4-yl)-2,5-diazabicyclo[2.2.2]octane-2-carboxylate (30 mg, 0.049 mmol), triisopropyl((6-(methoxymethoxy)-8-(4,4,5,5-tetramethyl-1,3,2-dioxaborolan-2-yl)naphthalen-1-yl)ethynyl)silane (48 mg, 0.098 mmol, LabNetwork), potassium phosphate (31 mg, 0.15 mmol, Sigma Aldrich Corporation), and cataCXium A Pd G3 (5.4 mg, 7.35 μmol, Sigma Aldrich Corporation). The vial was purged with nitrogen and then the reactants were suspended in degassed tetrahydrofuran (0.4 mL) and water (0.04 mL). The reaction was then sealed and heated to 60° C. After stirring overnight, the reaction was cooled to room temperature and concentrated under reduced pressure. The crude oil was then purified by column chromatography on silica gel, eluting with a gradient of 0-50% 3:1 EtOAc/EtOH (with 2% triethylamine) in heptane to provide tert-butyl (1S,4S)-5-(6,8-difluoro-2-(((2R,7aS)-2-fluorotetrahydro-1H-pyrrolizin-7a(5H)-yl)methoxy)-7-(3-(methoxymethoxy)-8-((triisopropylsilyl)ethynyl)naphthalen-1-yl)quinazolin-4-yl)-2,5-diazabicyclo[2.2.2]octane-2-carboxylate as an off-white solid. m/z (ESI, +ve ion): 900.4 (M+H)⁺.

Step 2: 4-(4-((1S,4S)-2,5-Diazabicyclo[2.2.2]octan-2-yl)-6,8-difluoro-2-(((2R,7aS)-2-fluorotetrahydro-1H-pyrrolizin-7a(5H)-yl)methoxy)quinazolin-7-yl)-5-((triisopropylsilyl)ethynyl)naphthalen-2-ol. tert-Butyl (1S,4S)-5-(6,8-difluoro-2-(((2R,7aS)-2-fluorotetrahydro-1H-pyrrolizin-7a(5H)-yl)methoxy)-7-(3-(methoxymethoxy)-8-((triisopropylsilyl)ethynyl)naphthalen-1-yl)quinazolin-4-yl)-2,5-diazabicyclo[2.2.2]octane-2-carboxylate was dissolved in MeCN (1.5 mL). The solution was cooled to 0° C. before HCl (4 M in 1,4-dioxane, 0.3 mL, 1.23 mmol, Sigma-Aldrich Corporation) was added. The reaction was stirred for 15 min. After an additional 30 min at rt, the reaction was concentrated under reduced pressure to provide 4-(4-((1S,4S)-2,5-diazabicyclo[2.2.2]octan-2-yl)-6,8-difluoro-2-(((2R,7aS)-2-fluorotetrahydro-1H-pyrrolizin-7a(5H)-yl)methoxy)quinazolin-7-yl)-5-((triisopropylsilyl)ethynyl)naphthalen-2-ol as a yellow solid. m/z (ESI, +ve ion): 755.8 (M+H)⁺.

Step 3: 4-(4-((1S,4S)-2,5-Diazabicyclo[2.2.2]octan-2-yl)-6,8-difluoro-2-(((2R,7aS)-2-fluorotetrahydro-1H-pyrrolizin-7a(5H)-yl)methoxy)quinazolin-7-yl)-5-ethynylnaphthalen-2-ol. 4-(4-((1S,4S)-2,5-Diazabicyclo[2.2.2]octan-2-yl)-6,8-difluoro-2-(((2R,7aS)-2-fluorotetrahydro-1H-pyrrolizin-7a(5H)-yl)methoxy)quinazolin-7-yl)-5-((triisopropylsilyl)ethynyl)naphthalen-2-ol was dissolved in DMF (1.5 mL). Cesium fluoride (74 mg, 0.49 mmol, Sigma-Aldrich Corporation) was added. The reaction was stirred overnight. The reaction was filtered through a syringe filter and purified by reverse phase HPLC to provide 4-(4-((1S,4S)-2,5-diazabicyclo[2.2.2]octan-2-yl)-6,8-difluoro-2-(((2R,7aS)-2-fluorotetrahydro-1H-pyrrolizin-7a(5H)-yl)methoxy)quinazolin-7-yl)-5-ethynylnaphthalen-2-ol as bis(2,2,2-trifluoroacetate) and off-white solid (14 mg, 0.017 mmol, 34% combined yield over 3 steps). m/z (ESI, +ve ion): 600.2 (M+H)⁺. ¹H NMR (400 MHz, METHANOL-d₄) δ ppm 7.85 (d, J=8.15 Hz, 1H) 7.74 (dt, J=9.98, 2.01 Hz, 1H) 7.51-7.56 (m, 1H) 7.43 (d, J=7.94 Hz, 1H) 7.36 (d, J=2.51 Hz, 1H) 7.07-7.11 (m, 1H) 5.44-5.66 (m, 1H) 5.09-5.16 (m, 1H) 4.60-4.73 (m, 2H) 4.47-4.57 (m, 1H) 4.33-4.43 (m, 1H) 3.81-4.07 (m, 5H) 3.55-3.64 (m, 1H) 3.40-3.52 (m, 1H) 2.96-3.07 (m, 1H) 2.57-2.74 (m, 2H) 2.21-2.56 (m, 5H) 2.04-2.20 (in, 3H).

TABLE 4
Additional Examples. Prepared in an Analogous Manner to Example 180.
Products were isolated as corresponding TFA salts.

| | | | MS | |
|---|---|---|---|---|
| | | | m/z | |
| | | | (ESI, | |
| Ex.# | Structure | Name | +ve ion) | ¹H NMR |
| 181 | 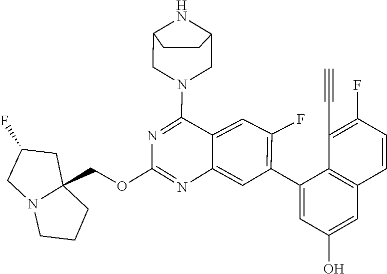 [SMILES: C#Cc1c(F)ccc2cc(O)cc(-c3cc4nc(OC[C@@]56CCCN5C[C@H](F)C6)nc(N5CC6CCC(C5)N6)c4cc3F)c12] | 4-(4-((1R,5S)-3,8- diazabicyclo[3.2.1] octan-3-yl)-6- fluoro-2- (((2R,7aS)-2- fluorotetrahydro- 1H-pyrrolizin- 7a(5H)- yl)methoxy) quinazolin- 7-yl)-5- ethynyl-6- fluoronaphthalen- 2-ol | 600.2 (M + H)⁺ | ¹H NMR (400 MHz, METHANOL-d₄) δ ppm 8.00 (s, 1 H), 7.87 (dd, J = 9.1, 5.7 Hz, 1 H), 7.71 (s, 1 H), 7.69 (d, J = 4.4 Hz, 1 H), 7.31-7.37 (m, 2 H), 7.09 (d, J = 2.5 Hz, 1 H), 5.46-5.77 (m, 1 H), 4.60-4.71 (m, 4 H), 4.27 (br s, 2 H), 3.80-4.04 (m, 5 H), 3.45- 3.54 (m, 1 H), 3.01 (s, 1 H), 2.86- 2.89 (m, 1 H), 2.56-2.80 (m, 2 H), 2.33-2.50 (m, 3 H), 2.15- 2.27 (m, 5 H). |
| 182 | 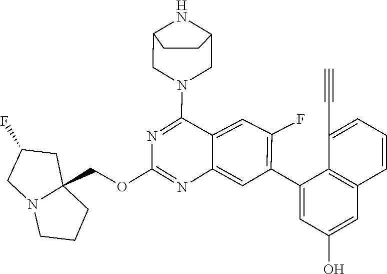 [SMILES: C#Cc1cccc2cc(O)cc(-c3cc4nc(OC[C@@]56CCCN5C[C@H](F)C6)nc(N5CC6CCC(C5)N6)c4cc3F)c12] | 4-(4-((1R,5S)-3,8- diazabicyclo[3.2.1] octan-3-yl)-6- fluoro-2- (((2R,7aS)-2- fluorotetrahydro- 1H-pyrrolizin- 7a(5H)- yl)methoxy) quinazolin- 7-yl)-5- ethynylnaphthalen- 2-ol | 582.2 (M + H)⁺ | ¹H NMR (400 MHz, METHANOL-d₄) δ ppm 8.00 (s, 1 H), 7.83 (dd, J = 8.3, 1.1 Hz, 1 H), 7.68-7.73 (m, 2 H), 7.53 (dd, J = 7.1, 1.3 Hz, 1 H), 7.41 (dd, J = 8.2, 7.3 Hz, 1 H), 7.32 (d, J = 2.5 Hz, 1 H), 7.05 (d, J = 2.7 Hz, 1 H), 5.47-5.74 (m, 1 H), 4.64-4.75 (m, 4 H), 4.25-4.32 (m, 2 H), 3.84-4.04 (m, 5 H), 3.46-3.54 (m, 1 H), 2.98-3.01 (m, 1 H), 2.88 (d, J = 0.6 Hz, 1 H), 2.58- 2.81 (m, 2 H), 2.33-2.51 (m, 3 H), 2.14-2.27 (m, 5 H). |
| 183 | 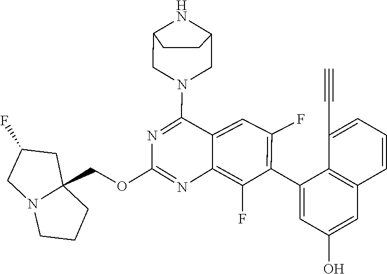 | 4-(4-((1R,5S)-3,6- diazabicyclo[3.1.1] heptan-3-yl)-6,8- difluoro-2- (((2R,7aS)-2- fluorotetrahydro- 1H-pyrrolizin- 7a(5H)- yl)methoxy) quinazolin- 7-yl)-5- ethynylnaphthalen- 2-ol | 585.9 (M + H)⁺ | ¹H NMR (METHANOL-d₄, 400 MHz) δ 7.84 (br d, 1H, J = 7.9 Hz), 7.5-7.6 (m, 2 H), 7.3-7.5 (m, 2 H), 7.09 (br s, 1 H), 5.4-5.7 (m, 1 H), 5.1-5.3 (m, 2 H), 4.69 (br s, 3 H), 4.0-4.1 (m, 2 H), 3.8-4.0 (m, 2 H), 3.6-3.7 (m, 2 H), 3.4-3.5 (m, 2 H), 3.0-3.1 (m, 1 H), 2.5-2.8 (m, 2 H), 2.3-2.5 (m, 3 H), 2.0-2.2 (m, 2 H). |
| 184 | 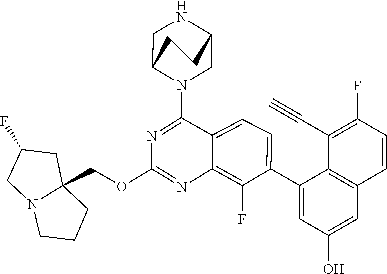 [SMILES: C#Cc1c(F)ccc2cc(O)cc(-c3ccc4c(N5C[C@@H]6CC[C@H]5CN6)nc(OC[C@@]56CCCN5C[C@H](F)C6)nc4c3F)c12] | 4-(5-((1S,4S)-2,5- diazabicyclo[2.2.2] octan-2-yl)-8- fluoro-2- (((2R,7aS)-2- fluorotetrahydro- 1H-pyrrolizin- 7a(5H)- yl)methoxy) quinazolin-7-yl)-5- ethynyl-6- fluoronaphthalen- 2-ol | 600.1 (M + H)⁺ | ¹H NMR (400 MHz, METHANOL-d₄) δ ppm 7.91 (dd, J = 8.6, 3.1 Hz, 1 H), 7.76-7.86 (m, 1 H), 7.17-7.34 (m, 3 H), 7.08 (t, J = 3.0 Hz, 1 H), 5.19-5.44 (m, 1 H), 4.87-4.94 (m, 1 H), 4.10-4.42 (m, 4 H), 3.35 (s, 2 H), 3.23 (br d, J = 0.8 Hz, 5 H), 2.99- 3.09 (m, 1 H), 1.86-2.41 (m, 10 H). |
| 185 | 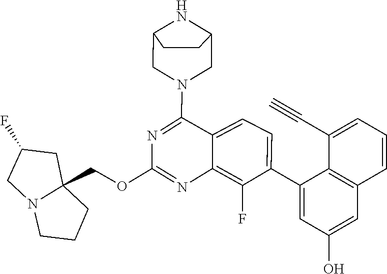 [SMILES: C#Cc1cccc2cc(O)cc(-c3ccc4c(N5CC6CCC(C5)N6)nc(OC[C@@]56CCCN5C[C@H](F)C6)nc4c3F)c12] | 4-(4-((1R,5S)-3,8- diazabicyclo[3.2.1] octan-3-yl)-8- fluoro-2- (((2R,7aS)-2- fluorotetrahydro- 1H-pyrrolizin- 7a(5H)- yl)methoxy) quinazolin-7-yl)-5- ethynylnaphthalen- 2-ol | 582.100 (M + H)⁺ | ¹H NMR (400 MHz, METHANOL-d₄) δ ppm 7.77- 7.92 (m, 2 H), 7.35-7.54 (m, 3 H), 7.28-7.33 (m, 1 H), 7.05 (s, 1 H), 5.44-5.63 (m, 1 H), 4.63- 4.72 (m, 3 H), 4.22-4.35 (m, 2 H), 3.75-4.08 (m, 5 H), 3.40- 3.56 (m, 1 H), 2.87-2.92 (m, 1 H), 2.28-2.50 (m, 3 H), 2.02- 2.78 (m, 11 H). |
| 186 | 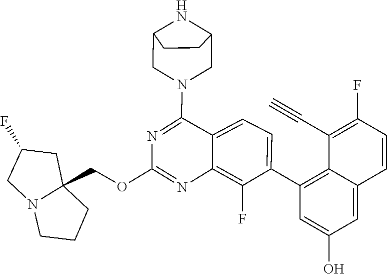 [SMILES: C#Cc1c(F)ccc2cc(O)cc(-c3ccc4c(N5CC6CCC(C5)N6)nc(OC[C@@]56CCCN5C[C@H](F)C6)nc4c3F)c12] | 4-(4-((1R,5S)-3,8- diazabicyclo[3.2.1] octan-3-yl)-8- fluoro-2- (((2R,7aS)-2- fluorotetrahydro- 1H-pyrrolizin- 7a(5H)- yl)methoxy) quinazolin-7-yl)-5- ethynyl-6- fluoronaphthalen- 2-ol | 600.1 (M + H)⁺ | ¹H NMR (400 MHz, METHANOL-d₄) δ ppm 7.81- 7.92 (m, 2 H), 7.40-7.48 (m, 1 H), 7.28-7.36 (m, 2 H), 7.07- 7.13 (m, 1 H), 5.44-5.67 (m, 1 H), 4.70 (s, 4 H), 4.20-4.34 (m, 2 H), 3.76-4.07 (m, 5 H), 3.41- 3.56 (m, 1 H), 3.21-3.26 (m, 1 H), 2.66-2.75 (m, 1 H), 2.55- 2.65 (m, 1 H), 2.28-2.50 (m, 3 H), 2.07-2.25 (m, 5 H). |
| 187 | 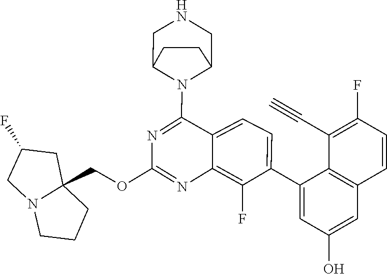 [SMILES: C#Cc1c(F)ccc2cc(O)cc(-c3ccc4c(N5C6CCC5CNC6)nc(OC[C@@]56CCCN5C[C@H](F)C6)nc4c3F)c12] | 4-(4-((1R,5S)-3,8- diazabicyclo[3.2.1] octan-8-yl)-8- fluoro-2- (((2R,7aS)-2- fluorotetrahydro- 1H-pyrrolizin- 7a(5H)- yl)methoxy) quinazolin- 7-yl)-5- ethynyl-6- fluoronaphthalen- 2-ol | 600.2 (M + H)⁺ | ¹H NMR (400 MHz, METHANOL-d₄) δ ppm 7.93- 8.02 (m, 1 H), 7.83-7.92 (m, 1 H), 7.43-7.54 (m, 1 H), 7.30- 7.40 (m, 2 H), 7.09-7.16 (m, 1 H), 5.46-5.65 (m, 1 H), 5.23- 5.36 (m, 2 H), 4.73 (s, 2 H), 3.81- 4.11 (m, 3 H), 3.63-3.78 (m, 2 H), 3.39-3.58 (m, 3 H), 3.18- 3.28 (m, 1 H), 2.54-2.82 (m, 2 H), 2.28-2.54 (m, 5 H), 2.18 (s, 3 H). |
| 188 | 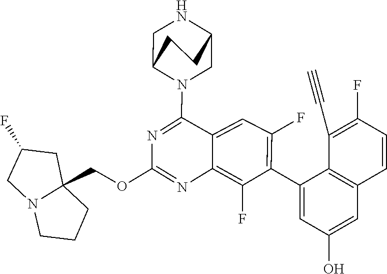 [SMILES: C#Cc1c(F)ccc2cc(O)cc(-c3c(F)cc4c(N5C[C@@H]6CC[C@H]5CN6)nc(OC[C@@]56CCCN5C[C@H](F)C6)nc4c3F)c12] | 4-(4-((15,4S)-2,5- diazabicyclo[2.2.2] octan-2-yl)-6,8- difluoro-2- (((2R,7aS)-2- fluorotetrahydro- 1H-pyrrolizin- 7a(5H)- yl)methoxy) quinazolin- 7-yl)-5- ethynyl-6- fluoronaphthalen- 2-ol | 618.2 (M + H)⁺ | ¹H NMR (400 MHz, METHANOL-d₄) δ ppm 7.86- 7.94 (m, 1 H) 7.72-7.78 (m, 1 H) 7.33-7.41 (m, 2 H) 7.14 (d, J = 2.51 Hz, 1 H) 5.48-5.70 (m, 1 H) 5.09-5.18 (m, 1 H) 4.63-4.75 (m, 2 H) 4.47-4.58 (m, 1 H) 4.33- 4.44 (m, 1 H) 3.82-4.11 (m, 5 H) 3.57-3.66 (m, 1 H) 3.43-3.54 (m, 1 H) 3.36-3.40 (m, 1 H) 2.67 (s, 2 H) 2.21-2.52 (m, 5 H) 2.01- 2.20 (m, 3 H). |
| 189 | 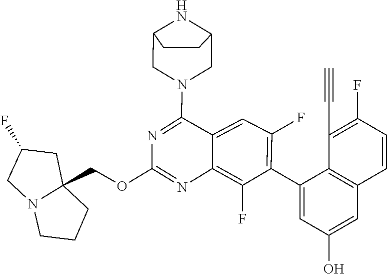 [SMILES: C#Cc1c(F)ccc2cc(O)cc(-c3c(F)cc4c(N5CC6CCC(C5)N6)nc(OC[C@@]56CCCN5C[C@H](F)C6)nc4c3F)c12] | 4-(4-((1R,5S)-3,8- diazabicyclo[3.2.1] octan-3-yl)-6,8- difluoro-2- (((2R,7aS)-2- fluorotetrahydro- 1H-pyrrolizin- 7a(5H)- yl)methoxy) quinazolin- 7-yl)-5- ethynyl-6- fluoronaphthalen- 2-ol | 618.2 (M + H)⁺ | ¹H NMR (400 MHz, METHANOL-d₄) δ ppm 7.85- 7.93 (m, 1 H) 7.59-7.65 (m, 1 H) 7.32-7.40 (m, 2 H) 7.14 (d, J = 2.51 Hz, 1 H) 5.47-5.68 (m, 1 H) 4.70 (br d, J = 1.67 Hz, 4 H) 4.23-4.32 (m, 2 H) 3.75-4.10 (m, 5 H) 3.44-3.54 (m, 1 H) 3.30- 3.31 (m, 1 H) 2.54-2.78 (m, 2 H) 2.31-2.50 (m, 3 H) 2.19 (br d, J = 8.15 Hz, 5 H). |
| 190 | 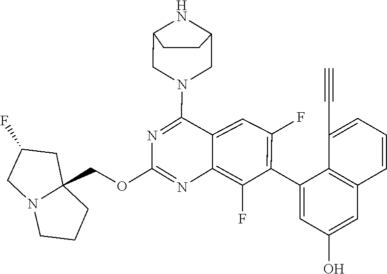 [SMILES: C#Cc1cccc2cc(O)cc(-c3c(F)cc4c(N5CC6CCC(C5)N6)nc(OC[C@@]56CCCN5C[C@H](F)C6)nc4c3F)c12] | 4-(4-((1R,5S)-3,8- diazabicyclo[3.2.1] octan-3-yl)-6,8- difluoro-2- (((2R,7aS)-2- fluorotetrahydro- 1H-pyrrolizin- 7a(5H)- yl)methoxy) quinazolin-7-yl)-5- ethynylnaphthalen- 2-ol | 600.2 (M + H)⁺ | ¹H NMR (400 MHz, METHANOL-d₄) δ ppm 7.85 (d, J = 8.36 Hz, 1 H) 7.61 (d, J = 9.82 Hz, 1 H) 7.53 (dd, J = 7.11, 1.25 Hz, 1 H) 7.42 (dd, J = 8.26, 7.21 Hz, 1 H) 7.36 (d, J = 2.51 Hz, 1 H) 7.09 (d, J = 2.51 Hz, 1 H) 5.45- 5.66 (m, 1 H) 4.71 (s, 4 H) 4.24- 4.32 (m, 2 H) 3.86-4.07 (m, 4 H) 3.77-3.85 (m, 1 H) 3.43-3.54 (m, 1 H) 2.98 (s, 1 H) 2.57-2.76 (m, 2 H) 2.29-2.50 (m, 3 H) 2.18 (br s, 5 H). |
| 191 | 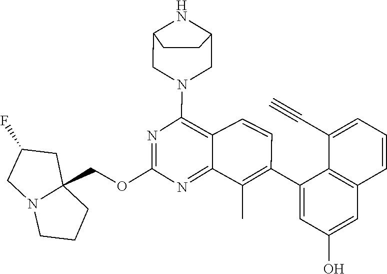 [SMILES: C#Cc1cccc2cc(O)cc(-c3ccc4c(N5CC6CCC(C5)N6)nc(OC[C@@]56CCCN5C[C@H](F)C6)nc4c3C)c12] | 4-(4-((1R,5S)-3,8- diazabicyclo[3.2.1] octan-3-yl)-2- (((2R,7aS)-2- fluorotetrahydro- 1H-pyrrolizin- 7a(5H)- yl)methoxy)-8- methylquinazolin- 7-yl)-5- ethynylnaphthalen- 2-ol | 578.3 (M + H)⁺ | ¹H NMR (400 MHz, METHANOL-d₄) δ ppm 7.86 (d, J = 8.4 Hz, 1 H), 0.00 (dd, J = 8.2, 1.3 Hz, 1 H), 7.48 (dd, J = 7.1, 1.3 Hz, 1 H), 7.34-7.41 (m, 2 H), 7.28 (d, J = 2.5 Hz, 1 H), 6.93 (d, J = 2.5 Hz, 1 H), 5.68 (td, J = 52.0, 3.6 Hz, 1 H), 4.74 (br s, 2 H), 4.59- 4.69 (m, 2 H), 4.27 (br s, 2 H), 3.81-4.09 (m, 5 H), 3.47-3.55 (m, 1 H), 2.60-2.85 (m, 3 H), 2.28 (s, 11 H). |

### 4-((1R,5S)-3,8-Diazabicyclo[3.2.1]octan-3-yl)-7-(6-chloro-5-methyl-1H-indazol-4-yl)-8-fluoro-2-(((2R,7aS)-2-fluorotetrahydro-1H-pyrrolizin-7a(5H)-yl)methoxy)quinazoline Example 192

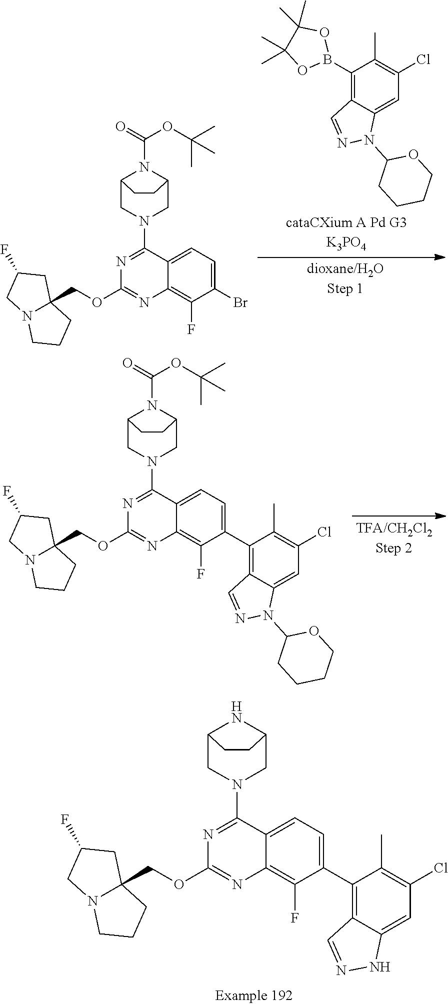

Step 1: tert-Butyl (1R,5S)-3-(7-(6-chloro-5-methyl-1-(tetrahydro-2H-pyran-2-yl)-1H-indazol-4-yl)-8-fluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate. A vial was charged with tert-butyl (1R,5S)-3-(7-bromo-8-fluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate (70 mg, 0.12 mmol), 6-chloro-5-methyl-1-(tetrahydro-2H-pyran-2-yl)-4-(4,4,5,5-tetramethyl-1,3,2-dioxaborolan-2-yl)-1H-indazole (89 mg, 0.24 mmol, PharmaBlock), potassium phosphate (55 mg, 0.26 mmol), and cataCXium A Pd G3 (15 mg, 0.024 mmol). The vial was purged with nitrogen gas and then the reactants were suspended in degassed 1,4-dioxane (1.0 mL) and water (0.2 mL). The reaction was then sealed and heated to 90° C. After stirring for 3 h, the crude material was purified by column chromatography on silica gel, eluting with a gradient of 0-100% 3:1 EtOAc/EtOH blend (with 1% triethylamine) in heptane to provide tert-butyl (1R,5S)-3-(7-(6-chloro-5-methyl-1-(tetrahydro-2H-pyran-2-yl)-1H-indazol-4-yl)-8-fluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate as tan solid, which was used directly in the subsequent step. m/z (ESI, +ve ion): 763.750 (M+H)⁺.

Step 2: 4-((1R,5S)-3,8-Diazabicyclo[3.2.1]octan-3-yl)-7-(6-chloro-5-methyl-1H-indazol-4-yl)-8-fluoro-2-(((2R,7aS)-2-fluorotetrahydro-1H-pyrrolizin-7a(5H)-yl)methoxy)quinazoline. tert-Butyl (1R,5S)-3-(7-(6-chloro-5-methyl-1-(tetrahydro-2H-pyran-2-yl)-1H-indazol-4-yl)-8-fluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate was dissolved in DCM (2 mL) and TFA (1 mL). The reaction was stirred at rt for 2 h. The mixture was purified by reverse phase HPLC to give 4-((1R,5S)-3,8-diazabicyclo[3.2.1]octan-3-yl)-7-(6-chloro-5-methyl-1H-indazol-4-yl)-8-fluoro-2-(((2R,7aS)-2-fluorotetrahydro-1H-pyrrolizin-7a(5H)-yl)methoxy)quinazoline as TFA salt and off-white solid (40 mg, 0.058 mmol, 49% combined yield over 2 steps). m/z (ESI, +ve ion): 580.2 (M+H)⁺. ¹H NMR (400 MHz, METHANOL-d₄) δ ppm 7.95-8.06 (m, 1H), 7.79 (s, 1H), 7.60 (s, 1H), 7.36-7.49 (m, 1H), 5.51-5.71 (m, 1H), 4.67-4.80 (m, 4H), 4.22-4.38 (m, 2H), 3.77-4.12 (m, 5H), 3.40-3.59 (m, 1H), 2.55-2.87 (m, 2H), 2.33 (s, 6H), 2.20 (br s, 5H).

TABLE 5
Additional Examples. Prepared in an Analogous Manner to Example 192.
Products were isolated as corresponding TFA salts.

| | | | MS | |
|---|---|---|---|---|
| | | | m/z | |
| | | | (ESI, | |
| Ex.# | Structure | Name | +ve ion) | ¹H NMR |
| 193 | 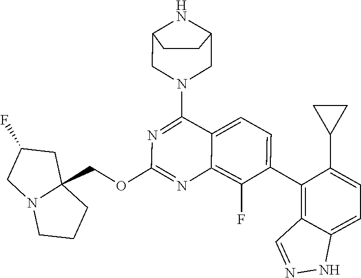 [SMILES: Fc1c(-c2c(C3CC3)ccc3[nH]ncc23)ccc2c(N3CC4CCC(C3)N4)nc(OC[C@@]34CCCN3C[C@H](F)C4)nc12] | 4-((1R,5S)-3,8- diazabicyclo[3.2.1] octan-3-yl)-7-(5- cyclopropyl-1H- indazol-4-yl)-8- fluoro-2- (((2R,7aS)-2- fluorotetrahydro- 1H-pyrrolizin- 7a(5H)- yl)methoxy) quinazoline | 572.2 (M + H)⁺ | ¹H NMR (400 MHz, METHANOL-d₄) δ ppm 7.85- 8.14 (m, 1 H), 7.56-7.66 (m, 2 H), 7.46-7.56 (m, 1 H), 7.18 (d, J = 8.9 Hz, 1 H), 5.22-5.79 (m, 1 H), 4.63-4.83 (m, 4 H), 4.23- 4.41 (m, 2 H), 3.77-4.11 (m, 5 H), 3.42-3.62 (m, 1 H), 2.57-2.80 (m, 2 H), 2.30-2.52 (m, 3 H), 2.20 (br s, 5 H), 1.80-1.95 (m, 1 H), 0.57-0.97 (m, 4 H). |
| 194 | 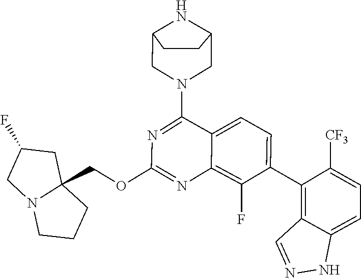 [SMILES: Fc1c(-c2c(C(F)(F)F)ccc3[nH]ncc23)ccc2c(N3CC4CCC(C3)N4)nc(OC[C@@]34CCCN3C[C@H](F)C4)nc12] | 4-((1R,5S)-3,8- diazabicyclo[3.2.1] octan-3-yl)-8- fluoro-2- (((2R,7aS)-2- fluorotetrahydro- 1H-pyrrolizin- 7a(5H)- yl)methoxy)-7-(5- (trifluoromethyl)- 1H-indazol-4- yl)quinazoline | 600.2 (M + H)⁺ | ¹H NMR (400 MHz, METHANOL-d₄) δ ppm 7.96- 8.07 (m, 1 H), 7.85 (s, 2 H), 7.69 (s, 1 H), 7.40-7.50 (m, 1 H), 5.38- 5.82 (m, 1 H), 4.74-4.81 (m, 4 H), 4.28 (br s, 2 H), 3.77-4.13 (m, 5 H), 3.42-3.59 (m, 1 H), 2.57-2.80 (m, 4 H), 2.33-2.50 (m, 2 H), 2.20-2.25 (m, 4 H). |
| 195 | 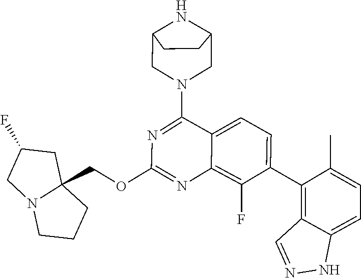 [SMILES: Cc1ccc2[nH]ncc2c1-c1ccc2c(N3CC4CCC(C3)N4)nc(OC[C@@]34CCCN3C[C@H](F)C4)nc2c1F] | 4-((1R,5S)-3,8- diazabicyclo[3.2.1] octan-3-yl)-8- fluoro-2- (((2R,7aS)-2- fluorotetrahydro- 1H-pyrrolizin- 7a(5H)- yl)methoxy)-7-(5- methyl-1H- indazol-4- yl)quinazoline | 546.2 (M + H)⁺ | ¹H NMR (400 MHz, METHANOL-d₄) δ ppm 7.89- 8.05 (m, 1 H), 7.60 (s, 2 H), 7.39- 7.50 (m, 2 H), 5.32-5.88 (m, 1 H), 4.65-4.80 (m, 4 H), 4.17- 4.41 (m, 2 H), 3.80-4.10 (m, 5 H), 3.44-3.58 (m, 1 H), 2.54- 2.84 (m, 2 H), 2.33-2.48 (m, 6 H), 2.20 (br s, 5 H). |
| 196 | 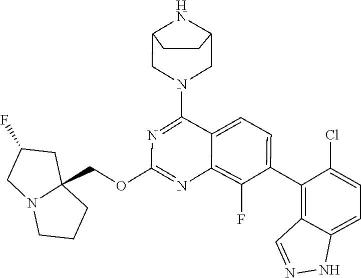 [SMILES: Fc1c(-c2c(Cl)ccc3[nH]ncc23)ccc2c(N3CC4CCC(C3)N4)nc(OC[C@@]34CCCN3C[C@H](F)C4)nc12] | 4-((1R,5S)-3,8- diazabicyclo[3.2.1] octan-3-yl)-7-(5- chloro-1H- indazol-4-yl)-8- fluoro-2- (((2R,7aS)-2- fluorotetrahydro- 1H-pyrrolizin- 7a(5H)- yl)methoxy) quinazoline | 566.2 (M + H)⁺ | ¹H NMR (400 MHz, METHANOL-d₄) δ ppm 7.93- 8.12 (m, 1 H), 7.63-7.75 (m, 2 H), 7.54-7.63 (m, 1 H), 7.32- 7.54 (m, 1 H), 5.39-5.80 (m, 1 H), 4.71-4.81 (m, 4 H), 4.18-4.40 (m, 2 H), 3.70-4.14 (m, 5 H), 3.41-3.60 (m, 1 H), 2.51-2.74 (m, 2 H), 2.27-2.52 (m, 3 H), 2.19 (br s, 5 H). |

### 4-(4-((1R,5S)-3,&-Diazabicyclo[3.2.1]octan-3-yl)-8fluoro-2-(((2R,7aS)-2-fluorotetrahydro-1H-pyrrolizin-7a(511)-yl)methoxy)quinazolin-7-yl)-1H-indazole-5-carbonitrile (Example 197

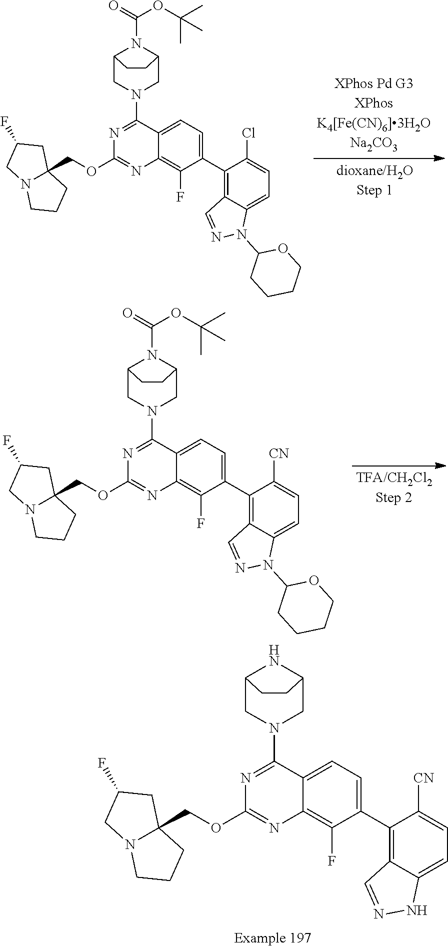

Step 1: tert-Butyl (1R,5S)-3-(7-(5-cyano-1-(tetrahydro-2H-pyran-2-yl)-1H-indazol-4-yl)-8-fluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate. To a 4-mL vial was added tert-butyl (1R,5S)-3-(7-(5-chloro-1-(tetrahydro-2H-pyran-2-yl)-1H-indazol-4-yl)-8-fluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate (30 mg, 0.040 mmol, synthesized in an analogous manner to Example 192), Na₂CO₃ (0.5 mg, 5.00 μmol), potassium ferrocyanide trihydrate (8.4 mg, 0.020 mmol), 2-dicyclohexylphosphino-2′,4′,6′,-triisopropylbiphenyl (3.8 mg, 8.00 μmol) and (2-dicyclohexylphosphino-2′,4′,6′-triisopropyl-1,1′-biphenyl)[2-(2′-amino-1,1′-biphenyl)]palladium(II) methanesulfonate (3.4 mg, 4.00 μmol) in dioxane (0.3 mL) and water (0.08 mL). The reaction was stirred at 90° C. for 4 h. The crude material was purified by column chromatography on silica gel, eluting with a gradient of 0-100% 3:1 EtOAc/EtOH (with 1% triethylamine) in heptane to provide tert-butyl (1R,5S)-3-(7-(5-cyano-1-(tetrahydro-2H-pyran-2-yl)-1H-indazol-4-yl)-8-fluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate, which was used directly in the subsequent step.

Step 2: 4-(4-((1R,5S)-3,8-Diazabicyclo[3.2.1]octan-3-yl)-8-fluoro-2-(((2R,7aS)-2-fluorotetrahydro-1H-pyrrolizin-7a(5H)-yl)methoxy)quinazolin-7-yl)-1H-indazole-5-carbonitrile. The above material was dissolved in DCM (2 mL) and TFA (1 mL). The reaction was stirred at rt for 2 h. The mixture was purified by reverse phase HPLC to give 4-(4-((1R,5S)-3,8-Diazabicyclo[3.2.1]octan-3-yl)-8-fluoro-2-(((2R,7aS)-2-fluorotetrahydro-1H-pyrrolizin-7a(5H)-yl)methoxy)quinazolin-7-yl)-1H-indazole-5-carbonitrile as TFA salt and white solid (10 mg, 0.015 mmol, 37% combined yield over 2 steps). m/z (ESI, +ve ion): 557.2 (M+H)⁺. ¹H NMR (400 MHz, METHANOL-d₄) δ ppm 7.92-8.17 (m, 2H), 7.84 (s, 1 H), 7.55-7.74 (m, 1H), 7.30-7.48 (m, 1H), 5.38-5.78 (m, 1H), 4.62-4.85 (m, 4H), 4.22-4.46 (m, 2H), 3.80-4.10 (m, 5H), 3.44-3.67 (m, 1H), 2.30-2.88 (m, 5H), 2.20 (br s, 5H).

### 4-((1R,5S)-3,8-Diazabicyclo[3.2.1]octan-3-yl)-8-fluoro-2-(((2R,7aS)-2-fluorotetrahydro-1H-pyrrolizin-7a(5H)-yl)methoxy)-7-(5-(prop-1-en-2-yl)-1H-indazol-4-yl)quinazoline Example 198

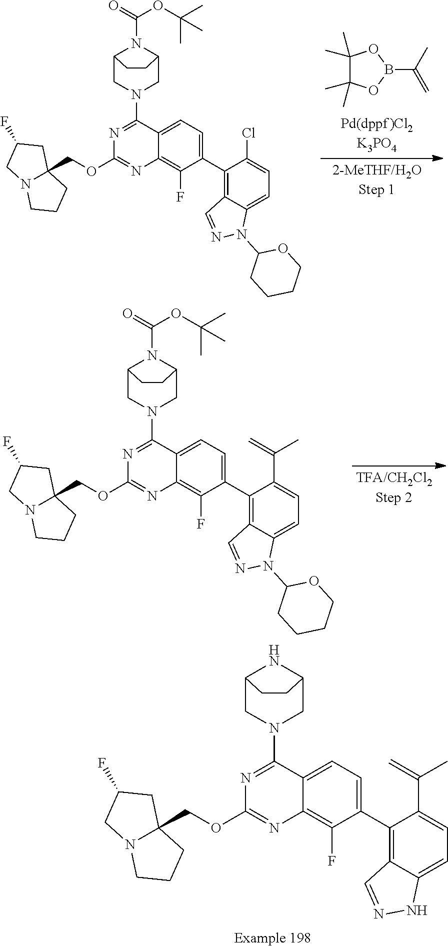

Step 1: tert-Butyl (1R,5S)-3-(8-fluoro-2-(((2R,7aS)-2-fluorotetrahydro-1H-pyrrolizin-7a(5H)-yl)methoxy)-7-(5-(prop-1-en-2-yl)-1-(tetrahydro-2H-pyran-2-yl)-1H-indazol-4-yl)quinazolin-4-yl)-3,8-diazabicyclo[3.2.1]octane-8-carboxylate. A 4-mL vial was charged with tert-butyl (1R,5S)-3-(7-(5-chloro-1-(tetrahydro-2H-pyran-2-yl)-1H-indazol-4-yl)-8-fluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate (50 mg, 0.067 mmol), 1,1′-bis(diphenylphosphino)ferrocene-palladium dichloride (4.9 mg, 6.66 μmol), potassium phosphate (50 mg, 0.23 mmol), and (4,4,5,5-tetramethyl-1,3,2-dioxaborolan-2-yl)isopropene (34 mg, 0.20 mmol, Combi-Blocks Inc.). The vial was purged with nitrogen and the reactants were suspended in 2-methyl THF (0.6 mL) and water (0.1 mL). The reaction was heated to 90° C. for 6 h. The crude material was purified by column chromatography on silica gel, eluting with a gradient of 0-100% 3:1 EtOAc/EtOH (with 1% triethylamine) in heptane to provide tert-butyl (1R,5S)-3-(8-fluoro-2-(((2R,7aS)-2-fluorotetrahydro-1H-pyrrolizin-7a(5H)-yl)methoxy)-7-(5-(prop-1-en-2-yl)-1-(tetrahydro-2H-pyran-2-yl)-1H-indazol-4-yl)quinazolin-4-yl)-3,8-diazabicyclo[3.2.1]octane-8-carboxylate, which was used directly in the subsequent step. m/z (ESI, +ve ion): 756.4 (M+H)⁺.

Step 2: 4-((1R,5S)-3,8-Diazabicyclo[3.2.1]octan-3-yl)-8-fluoro-2-(((2R,7aS)-2-fluorotetrahydro-1H-pyrrolizin-7a(5H)-yl)methoxy)-7-(5-(prop-1-en-2-yl)-1H-indazol-4-yl)quinazoline. The above material was dissolved in DCM (2 mL) and TFA (1 mL). The reaction was stirred at rt for 2 h. The reaction was concentrated under reduced pressure. The mixture was purified by reverse phase HPLC to give 4-((1R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)-7-(5-(prop-1-en-2-yl)-1H-indazol-4-yl)quinazoline as TFA salt as white solid (9 mg, 0.013 mmol, 19% combined yield over 2 steps). m/z (ESI, +ve ion): 572.2 (M+H)⁺. ¹H NMR (400 MHz, METHANOL-d4) δ ppm 8.90-9.20 (m, 1H), 8.22-8.42 (m, 1H), 7.86-8.13 (m, 1H), 7.63-7.68 (m, 1H), 7.61-7.77 (m, 1H), 7.48 (d, J=8.6 Hz, 1H), 5.48-5.83 (m, 1H), 4.96-5.21 (m, 1H), 4.84-4.95 (m, 1H), 4.61-4.76 (m, 5H), 4.20-4.39 (m, 2H), 3.79-4.16 (m, 5H), 3.41-3.67 (m, 1H), 3.00 (s, 3H), 2.25-2.51 (m, 4H), 2.20 (br s, 4H), 1.99 (s, 2H).

### 4-((1R,5S)-3,8-Diazabicyclo[3.2.1]octan-3-yl)-7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)quinazolin-6-ol (Example 199

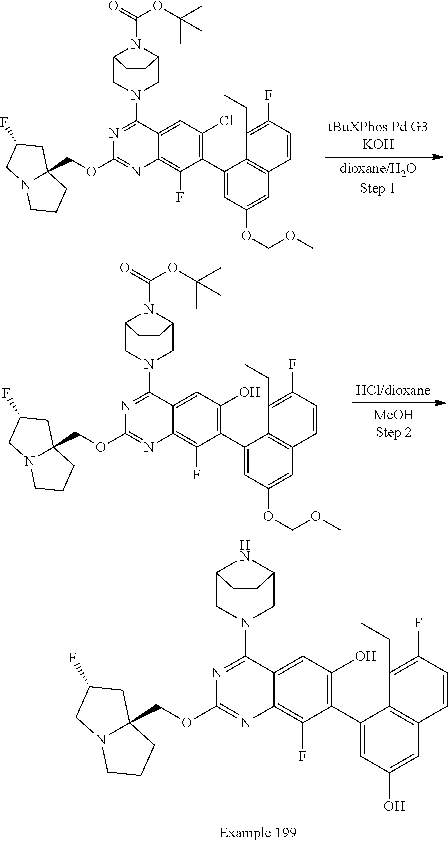 [SMILES: CCc1c(F)ccc2cc(OCOC)cc(-c3c(Cl)cc4c(N5CC6CCC(C5)N6C(=O)OC(C)(C)C)nc(OC[C@@]56CCCN5C[C@H](F)C6)nc4c3F)c12 ; CCc1c(F)ccc2cc(OCOC)cc(-c3c(O)cc4c(N5CC6CCC(C5)N6C(=O)OC(C)(C)C)nc(OC[C@@]56CCCN5C[C@H](F)C6)nc4c3F)c12 ; CCc1c(F)ccc2cc(O)cc(-c3c(O)cc4c(N5CC6CCC(C5)N6)nc(OC[C@@]56CCCN5C[C@H](F)C6)nc4c3F)c12]

Step 1: tert-Butyl (1R,5S)-3-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)-6-hydroxyquinazolin-4-yl)-3,8-diazabicyclo[3.2.1]octane-8-carboxylate. An 8-mL vial was charged with potassium hydroxide (43 mg, 0.77 mmol), [(2-di-tert-butylphosphino-2′,4′,6′-triisopropyl-1,1′-biphenyl)-2-(2′-amino-1,1′-biphenyl)]palladium(II) methanesulfonate (41 mg, 0.051 mmol), tert-butyl (1R,5S)-3-(6-chloro-7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate (0.20 g, 0.26 mmol, synthesized in an analogous manner to Example 109), 1,4-dioxane (0.6 mL) and water (0.6 mL). The reaction was stirred at 100° C. for 1 h. The crude product was purified by column chromatography on silica gel, eluting with 0-50% 3:1 EtOAc/EtOH blend in heptane with 2% triethylamine additive to yield tert-butyl (1R,5S)-3-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)-6-hydroxyquinazolin-4-yl)-3,8-diazabicyclo[3.2.1]octane-8-carboxylate (66 mg, 0.086 mmol, 34% yield). m/z (ESI, +ve ion): 764.2 (M+H)⁺.

Step 2: 4-((1R,5S)-3,8-Diazabicyclo[3.2.1]octan-3-yl)-7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)quinazolin-6-ol. tert-Butyl (1R,5S)-3-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)-6-hydroxyquinazolin-4-yl)-3,8-diazabicyclo[3.2.1]octane-8-carboxylate (66 mg, 0.086 mmol) was stirred in HCl solution (4.0 M in dioxane, 0.4 mL, 1.73 mmol) and methanol (0.4 mL) at rt for 1 h. Solvents were removed under reduced pressure. The crude product was purified by reverse phase HPLC to yield 4-((1R,5S)-3,8-diazabicyclo[3.2.1]octan-3-yl)-7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)quinazolin-6-ol as bis(2,2,2-trifluoroacetate) (35 mg, 0.041 mmol, 47% yield). m/z (ESI, +ve ion): 620.2 (M+H)⁺. ¹H NMR (400 MHz, METHANOL-d₄) δ ppm 7.61-7.73 (m, 1H), 7.18-7.34 (m, 3H), 6.87-6.96 (m, 1H), 5.44-5.71 (m, 1H), 4.51-4.73 (m, 4H), 4.21-4.36 (m, 2H), 3.70-4.10 (m, 5H), 3.40-3.57 (m, 1H), 2.20-2.59 (m, 12H), 0.71-0.91 (m, 3H).

TABLE 6
Additional Examples. Prepared in an Analogous Manner to Example 199.
Products were isolated as corresponding TFA salts.

| | | | MS | |
|---|---|---|---|---|
| | | | m/z | |
| | | | (ESI, | |
| Ex. # | Structure | Name | +ve ion) | ¹H NMR |
| 200 | 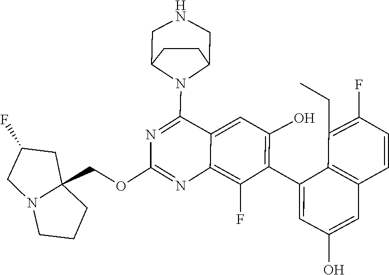 | 4-((1R,5S)-3,8- diazabicyclo[3.2.1] octan-8-yl)-7-(8- ethyl-7-fluoro-3- hydroxynaphthalen- 1-yl)-8-fluoro-2- (((2R,7aS)-2- fluorotetrahydro- 1H-pyrrolizin- 7a(5H)- yl)methoxy) quinazolin-6-ol | 549.2 (M + H)⁺ | ¹H NMR (400 MHz, METHANOL-d₄) δ ppm 7.62- 7.72 (m, 1 H), 7.32-7.41 (m, 1 H), 7.19-7.29 (m, 2 H), 6.87- 6.97 (m, 1 H), 5.45-5.67 (m, 1 H), 5.14-5.23 (m, 2 H), 4.62- 4.72 (m, 2 H), 3.80-4.10 (m, 3 H), 3.62-3.77 (m, 2 H), 3.42- 3.57 (m, 3 H), 2.27-2.81 (m, 9 H), 2.08-2.25 (m, 3 H), 0.77- 0.86 (m, 3 H). |
| 201 | 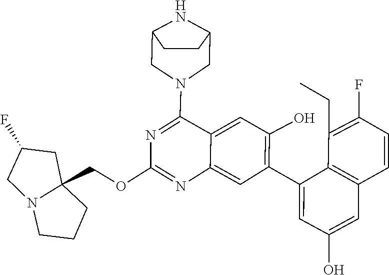 [SMILES: CCc1c(F)ccc2cc(O)cc(-c3cc4nc(OC[C@@]56CCCN5C[C@H](F)C6)nc(N5CC6CCC(C5)N6)c4cc3O)c12] | 4-((1R,5S)-3,8- diazabicyclo[3.2.1] octan-3-yl)-7-(8- ethyl-7-fluoro-3- hydroxynaphthalen- 1-yl)-2- (((2R,7aS)-2- fluorotetrahydro- 1H-pyrrolizin- 7a(5H)- yl)methoxy) quinazolin-6-ol | 602.2 (M + H)⁺ | ¹H NMR (400 MHz, METHANOL-d₄) δ ppm 7.58- 7.73 (m, 2 H), 7.41-7.50 (m, 1 H), 7.14-7.31 (m, 2 H), 6.85- 6.96 (m, 1 H), 5.47-5.75 (m, 1 H), 4.74 (s, 4 H), 4.23-4.39 (m, 2 H), 3.80-4.07 (m, 5 H), 3.43- 3.59 (m, 1 H), 2.55-2.84 (m, 3 H), 2.32-2.52 (m, 4 H), 2.12- 2.31 (m, 5 H), 0.71-0.87 (m, 3 H). |

### 4-(6-Amino 4-((1R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)quinazolin-7-yl)-5-ethyl-6-fluoronaphthalen-2-ol (Example 202

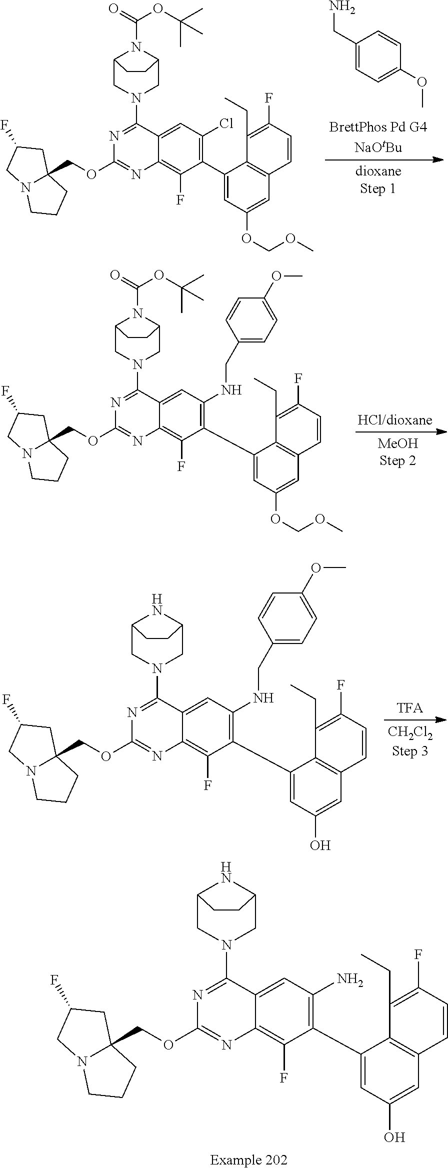

Step 1: tert-Butyl (1R,5S)-3-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)-6-((4-methoxybenzyl)amino)quinazolin-4-yl)-3,8-diazabicyclo[3.2.1]octane-8-carboxylate. An 8-mL vial was charged with 4-methoxybenzylamine (70 mg, 0.07 mL, 0.51 mmol), sodium tert-butoxide (74 mg, 0.77 mmol), BrettPhos Pd G4 (47 mg, 0.051 mmol, Sigma Aldrich Corporation), tert-butyl (1R,5S)-3-(6-chloro-7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate (0.20 g, 0.26 mmol, synthesized in an analogous manner to Example 109) and 1,4-dioxane (2.6 mL). The reaction was stirred at 100° C. for 3 h. The crude mixture was purified by column chromatography on silica gel, eluting with 0-50% 3:1 EtOAc/EtOH in heptane with 2% triethylamine additive to yield tert-butyl (1R,5S)-3-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)-6-((4-methoxybenzyl)amino)quinazolin-4-yl)-3,8-diazabicyclo[3.2.1]octane-8-carboxylate (38 mg, 0.043 mmol, 17% yield). m/z (ESI, +ve ion): 883.4 (M+H)⁺.

Step 2: 4-(4-((1R,5S)-3,8-Diazabicyclo[3.2.1]octan-3-yl)-8-fluoro-2-(((2R,7aS)-2-fluorotetrahydro-1H-pyrrolizin-7a(5H)-yl)methoxy)-6-((4-methoxybenzyl)amino)quinazolin-7-yl)-5-ethyl-6-fluoronaphthalen-2-ol. tert-Butyl (1R,5S)-3-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)-6-((4-methoxybenzyl)amino)quinazolin-4-yl)-3,8-diazabicyclo[3.2.1]octane-8-carboxylate (38 mg, 0.043 mmol) was stirred in HCl solution (4.0 M in dioxane, 0.2 mL, 0.86 mmol) and methanol (0.2 mL) at rt for 1 h. Solvents were removed under reduced pressure. The crude product was used in the subsequent step without further manipulation. m/z (ESI, +ve ion): 739.4 (M+H)⁺.

Step 3: 4-(6-Amino-4-((1R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)quinazolin-7-yl)-5-ethyl-6-fluoronaphthalen-2-ol. The crude 4-(4-((1R,5S)-3,8-Diazabicyclo[3.2.1]octan-3-yl)-8-fluoro-2-(((2R,7aS)-2-fluorotetrahydro-1H-pyrrolizin-7a(5H)-yl)methoxy)-6-((4-methoxybenzyl)amino)quinazolin-7-yl)-5-ethyl-6-fluoronaphthalen-2-ol was stirred in trifluoroacetic acid (0.2 mL) and dichloromethane (0.2 mL) at rt for 2 h. Solvents were then removed under reduced pressure. The crude product was purified by reverse phase HPLC to yield 4-(6-amino-4-((1R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)quinazolin-7-yl)-5-ethyl-6-fluoronaphthalen-2-ol bis(2,2,2-trifluoroacetate) (11 mg, 0.013 mmol, 30% combined yield over two steps). m/z (ESI, +ve ion): 619.2 (M+H)⁺. ¹H NMR (400 MHz, METHANOL-d₄) δ ppm 7.67-7.74 (m, 1H), 7.32-7.35 (m, 1H), 7.23-7.31 (m, 1H), 7.11-7.16 (m, 1H), 6.94-6.98 (m, 1H), 5.43-5.65 (m, 1H), 4.53-4.71 (m, 3H), 4.23-4.37 (m, 2H), 3.69-4.07 (m, 5H), 3.39-3.55 (m, 1H), 2.57-2.78 (m, 3H), 2.02-2.56 (m, 10H), 0.84 (s, 3H).

### 4-(6-Amino-4-((1R,5S)-3,8-diazabicyclo[3.2.1]octan-3-yl)-2-(((2R,7aS)-2-fluorotetrahydro-1H-pyrrolizin-7a(5H)-yl)methoxy)quinazolin-7-yl)-5-ethyl-6-fluoronaphthalen-2-ol (Example 203

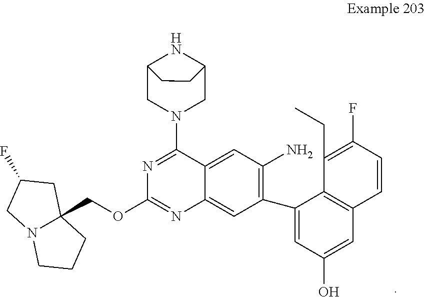 [SMILES: CCc1c(F)ccc2cc(O)cc(-c3cc4nc(OC[C@@]56CCCN5C[C@H](F)C6)nc(N5CC6CCC(C5)N6)c4cc3N)c12]

Synthesized in an analogous manner to Example 202. The product was isolated as a bis(2,2,2-trifluoroacetate) salt. m/z (ESI, +ve ion): 601.0 (M+H)⁺. ¹H NMR (400 MHz, METHANOL-d₄) δ ppm 7.64-7.75 (m, 1H), 7.48-7.54 (m, 1H), 7.34-7.38 (m, 1H), 7.21-7.32 (m, 2H), 6.89-6.97 (m, 1H), 5.49-5.72 (m, 1H), 4.79-4.87 (m, 4H), 4.26-4.37 (m, 2H), 3.84-4.06 (m, 5H), 3.42-3.58 (m, 1H), 2.59-2.82 (m, 3H), 2.33-2.57 (m, 4H), 2.13-2.30 (m, 5H), 0.77-0.86 (m, 3H).

### 8-(4-((1R,5S)-3,8-Diazabicyclo[3.2.1]octan-3-yl)-6,8-difluoro-2-(((2R,7aS)-2-fluorotetrahydro-1H-pyrrolizin-7a(5H)-yl)methoxy)quinazolin-7-yl)-6-hydroxy-1-naphthonitrile (Example 204

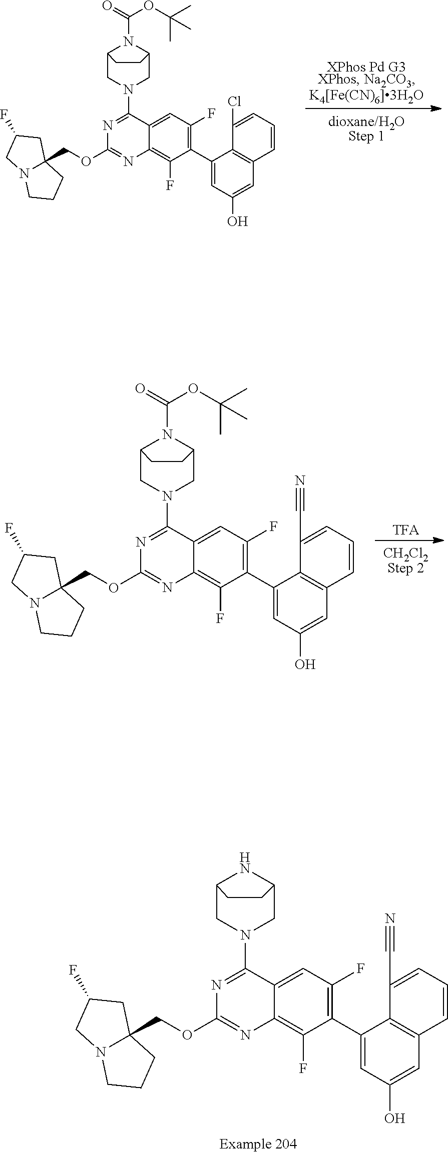

Step 1: tert-Butyl (1R,5S)-3-(7-(8-cyano-3-hydroxynaphthalen-1-yl)-6,8-difluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate. tert-Butyl (1R,5S)-3-(7-(8-chloro-3-hydroxynaphthalen-1-yl)-6,8-difluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate (0.10 g, 0.14 mmol, synthesized in an analogous manner to Example 109), Na₂CO₃ (1.9 mg, 0.018 mmol), 2-dicyclohexylphosphino-2′,4′,6′,-triisopropylbiphenyl (13 mg, 0.028 mmol), (2-dicyclohexylphosphino-2′,4′,6′-triisopropyl-1,1′-biphenyl)[2-(2′-amino-1,1′-biphenyl)]palladium(II) methanesulfonate (12 mg, 0.014 mmol, Sigma Aldrich Corporation) and potassium ferrocyanide trihydrate (30 mg, 0.070 mmol, Toronto Research Chemicals) were suspended in degassed dioxane (0.7 mL) and water (0.7 mL) and stirred at 90° C. for 90 min. After cooling to rt, water (3 mL) was added and the mixture was extracted with EtOAc (3×5 mL). The combined organic layer was dried over anhydrous sodium sulfate. Volatiles were removed under reduced pressure, and the residue was purified via column chromatography on silica gel, eluting with a gradient of 0-100% 3:1 EtOAc/EtOH (with 2% triethylamine) in heptane to yield tert-butyl (1R,5S)-3-(7-(8-cyano-3-hydroxynaphthalen-1-yl)-6,8-difluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate (0.10 g) as shiny white solid, which was used directly in the next step without being fully dried.

Step 2: 8-(4-((1R,5S)-3,8-Diazabicyclo[3.2.1]octan-3-yl)-6,8-difluoro-2-(((2R,7aS)-2-fluorotetrahydro-1H-pyrrolizin-7a(5H)-yl)methoxy)quinazolin-7-yl)-6-hydroxy-1-naphthonitrile. The above product tert-butyl (1R,5S)-3-(7-(8-cyano-3-hydroxynaphthalen-1-yl)-6,8-difluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate was suspended in DCM (1.8 mL) and TFA was added (0.3 mL). The mixture was stirred at rt for 2 h. Volatiles were removed under reduced pressure and the residue was purified by reverse phase HPLC to yield 8-(4-((1R,5S)-3,8-diazabicyclo[3.2.1]octan-3-yl)-6,8-difluoro-2-(((2R,7aS)-2-fluorotetrahydro-1H-pyrrolizin-7a(5H)-yl)methoxy)quinazolin-7-yl)-6-hydroxy-1-naphthonitrile as bis(2,2,2-trifluoroacetate) (44 mg, 0.053 mmol, 38% yield). m/z (ESI, +ve ion): 601.0 (M+H)⁺. ¹H NMR (400 MHz, METHANOL-d₄) δ ppm 8.16 (dd, J=8.5, 0.9 Hz, 1H), 7.77-7.86 (m, 1H), 7.72 (br d, J=9.8 Hz, 1H), 7.59 (dd, J=8.4, 7.3 Hz, 1H), 7.47 (d, J=2.5 Hz, 1H), 7.28 (d, J=2.3 Hz, 1H), 5.50-5.68 (m, 1H), 4.63-4.79 (m, 5H), 4.25-4.31 (m, 2H), 3.85-4.09 (m, 5H), 3.44-3.54 (m, 1H), 2.32-2.80 (m, 5H), 2.18 (br s, 5H).

### 8-(4-((1R,5S)-3,8-Diazabicyclo[3.2.1]octan-3-yl)-8-fluoro-2-(((2R,7aS)-2-fluorotetrahydro-1H-pyrrolizin-7a(5H)-yl)methoxy)quinazolin-7-yl)-6-hydroxy-1-naphthonitrile (Example 205

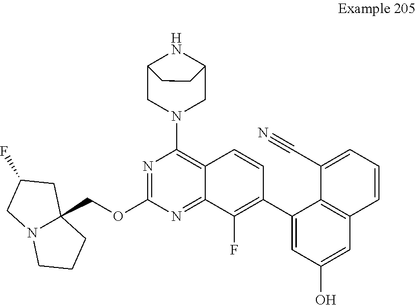 [SMILES: N#Cc1cccc2cc(O)cc(-c3ccc4c(N5CC6CCC(C5)N6)nc(OC[C@@]56CCCN5C[C@H](F)C6)nc4c3F)c12]

Synthesized in an analogous manner to Example 204. The product was isolated as the corresponding bis(2,2,2-trifluoroacetate). m/z (ESI, +ve ion): 583.2 (M+H)⁺. ¹H NMR (400 MHz, METHANOL-d₄) δ ppm 8.00 (d, J=8.4 Hz, 1H), 7.82 (d, J=8.8 Hz, 1H), 7.44 (d, J=1.0 Hz, 1H), 7.41 (d, J=6.7 Hz, 1H), 7.13-7.29 (m, 3H), 5.35 (dd, J=53.9, 1.0 Hz, 1H), 4.46-4.64 (m, 1H), 4.20-4.35 (m, 2H), 3.49-3.71 (m, 4H), 3.14-3.28 (m, 3H), 2.95-3.06 (m, 1H), 2.58 (br d, J=6.7 Hz, 7H), 1.81-2.05 (m, 7H).

### 4-(4-((1R,4R)-2,5-Diazabicyclo[2.2.2]octan-2-yl)-6,8-difluoro-2-(((2R,7aS)-2-fluorotetrahydro-1H-pyrrolizin-7a(5H)-yl)methoxy)quinazolin-7-yl)-1-chloronaphthalen-2-ol (Example 206

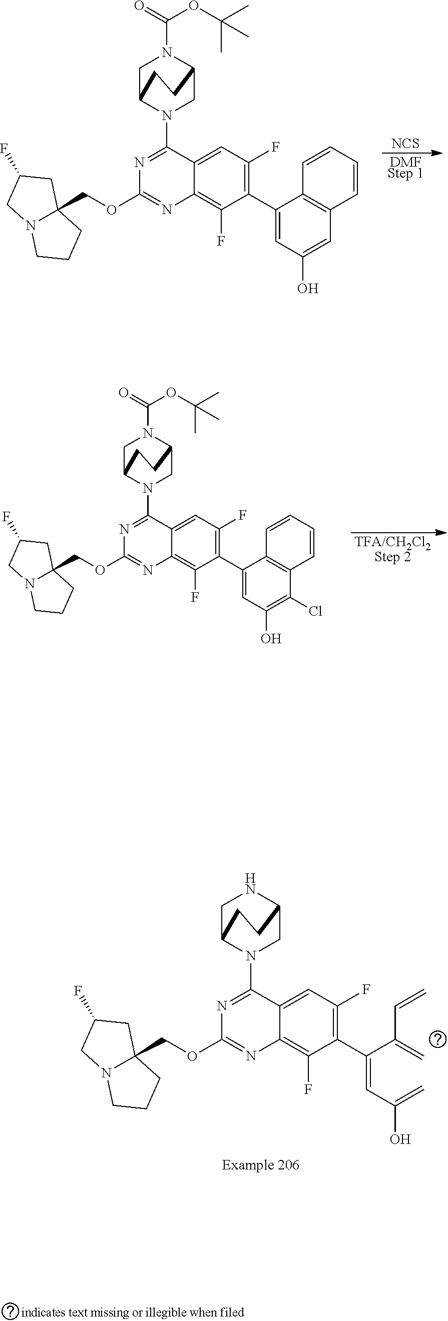

Step 1: tert-Butyl (1R,4R)-5-(7-(4-chloro-3-hydroxynaphthalen-1-yl)-6,8-difluoro-2-(((2R,7aS)-2-fluorotetrahydro-1H-pyrrolizin-7a(5H)-yl)methoxy)quinazolin-4-yl)-2,5-diazabicyclo[2.2.2]octane-2-carboxylate. To a stirred solution of tert-butyl (1R,4R)-5-(6,8-difluoro-2-(((2R,7aS)-2-fluorotetrahydro-1H-pyrrolizin-7a(5H)-yl)methoxy)-7-(3-hydroxynaphthalen-1-yl)quinazolin-4-yl)-2,5-diazabicyclo[2.2.2]octane-2-carboxylate (47 mg, 0.070 mmol, synthesized in an analogous manner to Example 109) in DMF (0.8 mL) was added N-chlorosuccinimide (9.3 mg, 0.070 mmol) at rt. The resulting mixture was stirred at rt for 2.5 h. The crude mixture was purified by column chromatography on silica gel, eluting with a gradient of 30-100% 3:1 EtOH/EtOAc in heptane to give tert-butyl (1R,4R)-5-(7-(4-chloro-3-hydroxynaphthalen-1-yl)-6,8-difluoro-2-(((2R,7aS)-2-fluorotetrahydro-1H-pyrrolizin-7a(5H)-yl)methoxy)quinazolin-4-yl)-2,5-diazabicyclo[2.2.2]octane-2-carboxylate, as colorless film, which was directly taken onto the next step. m/z (ESI, +ve ion): 710.2 (M+H)⁺.

Step 2: 4-(4-((1R,4R)-2,5-Diazabicyclo[2.2.2]octan-2-yl)-6,8-difluoro-2-(((2R,7aS)-2-fluorotetrahydro-1H-pyrrolizin-7a(5H)-yl)methoxy)quinazolin-7-yl)-1-chloronaphthalen-2-ol. To a stirred solution of a mixture of tert-butyl (1R,4R)-5-(7-(4-chloro-3-hydroxynaphthalen-1-yl)-6,8-difluoro-2-(((2R,7aS)-2-fluorotetrahydro-1H-pyrrolizin-7a(5H)-yl)methoxy)quinazolin-4-yl)-2,5-diazabicyclo[2.2.2]octane-2-carboxylate (30 mg, 0.042 mmol) in DCM (1.0 mL) was added TFA (4.8 mg, 1.0 mL, 0.042 mmol) at rt. The resulting mixture was stirred at rt for 2 h. Volatiles were removed and the residue was dissolved in MeOH and was purified by reverse phase HPLC to give a mixture as off-white solid, which was dissolved in MeOH/DCM and neutralized with ammonium hydroxide (commercial). The mixture was purified again by column chromatography on silica gel, eluting with a gradient of 1-20% (20% MeOH in DCM with 0.5% ammonium hydroxide) in DCM to give 4-(4-((1R,4R)-2,5-diazabicyclo[2.2.2]octan-2-yl)-6,8-difluoro-2-(((2R,7aS)-2-fluorotetrahydro-1H-pyrrolizin-7a(5H)-yl)methoxy)quinazolin-7-yl)-1-chloronaphthalen-2-ol as an off-white solid (2.7 mg, 0.004 mmol, 10% yield). m/z (ESI, +ve ion): 610.2 (M+H)⁺. ¹H NMR (METHANOL-d₄, 400 MHz) δ 8.22 (d, 1H, J=8.6 Hz), 7.83 (dd, 1H, J=1.7, 10.7 Hz), 7.57 (ddd, 1H, J=1.3, 6.8, 8.4 Hz), 7.38 (br d, 1H, J=8.2 Hz), 7.3-7.3 (m, 1H), 7.24 (s, 1H), 5.2-5.4 (m, 1H), 4.88 (br s, 1H), 4.2-4.4 (m, 2H), 4.1-4.2 (m, 2H), 3.47 (td, 1H, J=2.5, 11.5 Hz), 3.1-3.3 (m, 5H), 3.00 (dt, 1H, J=5.9, 9.3 Hz), 2.2-2.4 (m, 3H), 2.0-2.1 (m, 5H), 1.8-2.0 (m, 2H).

### 4-((1R,5S)-3,8-Diazabicyclo[3.2.1]octan-3-yl)-6-chloro-7-(3,5-dimethyl-1H-indazol-4-yl)-8-fluoro-2-(((2R,7aS)-2-fluorotetrahydro-1H-pyrrolizin-7a(5H)-yl)methoxy)quinazoline Example 207

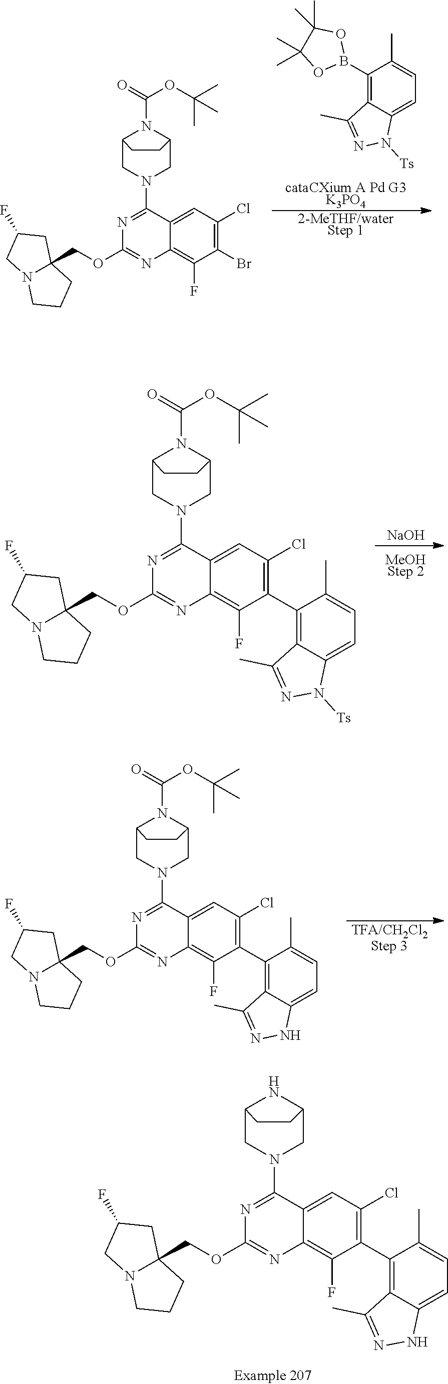

Step 1: tert-Butyl (1R,5S)-3-(6-chloro-7-(3,5-dimethyl-1-tosyl-1H-indazol-4-yl)-8-fluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate. A microwave vial was charged with tert-butyl (1R,5S)-3-(7-bromo-6-chloro-8-fluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate (50 mg, 0.079 mmol), 3,5-dimethyl-4-(4,4,5,5-tetramethyl-1,3,2-dioxaborolan-2-yl)-1-tosyl-1H-indazole (85 mg, 0.20 mmol, CombiBlocks), potassium phosphate (59 mg, 0.28 mmol), and cataCXium A Pd G3 (8.7 mg, 0.012 mmol, Sigma Aldrich Corporation). The vial was purged with nitrogen gas and then the reactants were suspended in 2-methyl THF (0.7 mL) and water (0.1 mL). The reaction was then sealed and heated to 115° C. under microwave for 120 min. The crude material was purified by column chromatography on silica gel, eluting with a gradient of 0-100% 3:1 EtOAc/EtOH in heptane with 1% triethylamine additive to provide tert-butyl (1R,5S)-3-(6-chloro-7-(3,5-dimethyl-1-tosyl-1H-indazol-4-yl)-8-fluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate, which was used directly in the subsequent step. m/z (ESI, +ve ion): 848.2 (M+H)⁺.

Step 2: tert-Butyl (1R,5S)-3-(6-chloro-7-(3,5-dimethyl-1H-indazol-4-yl)-8-fluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate. To a solution of the above tert-butyl (1R,5S)-3-(6-chloro-7-(3,5-dimethyl-1-tosyl-1H-indazol-4-yl)-8-fluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate in MeOH (2 mL) was added 0.5 mL of 10 N NaOH solution. The reaction was stirred at rt for 3 h. The reaction mixture was diluted with saturated NaCl solution (5 mL) and extracted with EtOAc (2×10 mL). The combined organic layer was dried over MgSO₄. The solution was filtered and concentrated in vacuo to give crude product, which was further purified by reverse phase HPLC to give tert-butyl (1R,5S)-3-(6-chloro-7-(3,5-dimethyl-1H-indazol-4-yl)-8-fluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate (10 mg, 0.014 mmol, 18% combined yield over 2 steps). m/z (ESI, +ve ion): 694.2 (M+H)⁺.

Step 3: 4-((1R,5S)-3,8-Diazabicyclo[3.2.1]octan-3-yl)-6-chloro-7-(3,5-dimethyl-1H-indazol-4-yl)-8-fluoro-2-(((2R,7aS)-2-fluorotetrahydro-1H-pyrrolizin-7a(5H)-yl)methoxy)quinazoline. A solution of tert-butyl (1R,5S)-3-(6-chloro-7-(3,5-dimethyl-1H-indazol-4-yl)-8-fluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate (10 mg, 0.014 mmol) in DCM (2 mL) was treated with 1 mL of TFA. The reaction was stirred at rt for 2 h. The mixture was concentrated under reduced pressure. The crude product was purified by reverse phase HPLC to give 4-((1R,5S)-3,8-diazabicyclo[3.2.1]octan-3-yl)-6-chloro-7-(3,5-dimethyl-1H-indazol-4-yl)-8-fluoro-2-(((2R,7aS)-2-fluorotetrahydro-1H-pyrrolizin-7a(5H)-yl)methoxy)quinazoline as a TFA salt and white solid (3.8 mg, 0.005 mmol, 36% yield). m/z (ESI, +ve ion): 594.2 (M+H)⁺. ¹H NMR (400 MHz, METHANOL-d₄) δ ppm 7.94-8.15 (m, 1H), 7.50-7.59 (m, 1H), 7.30-7.46 (m, 1H), 5.57-5.83 (m, 1H), 5.41-5.81 (m, 1H), 4.70 (br d, J=2.9 Hz, 3H), 4.18-4.36 (m, 2H), 3.81-4.13 (m, 5H), 3.44-3.67 (m, 1H), 2.54-2.89 (m, 2H), 2.29-2.50 (m, 3H), 2.19 (s, 8H), 1.91 (d, J=1.9 Hz, 3H).

TABLE 7
Additional Examples. Prepared in an Analogous Manner to Example 207.
Products were isolated as corresponding TFA salts.

| | | | MS | |
|---|---|---|---|---|
| | | | m/z | |
| | | | (ESI, | |
| Ex. # | Structure | Name | +ve ion) | ¹H NMR |
| 208 | 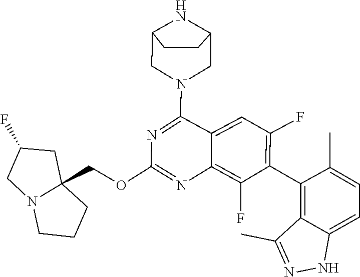 [SMILES: Cc1ccc2[nH]nc(C)c2c1-c1c(F)cc2c(N3CC4CCC(C3)N4)nc(OC[C@@]34CCCN3C[C@H](F)C4)nc2c1F] | 4-((1R,5S)-3,8- diazabicyclo[3.2.1] octan-3-yl)-7- (3,5-dimethyl-1H- indazol-4-yl)-6,8- difluoro-2- (((2R,7aS)-2- fluorotetrahydro- 1H-pyrrolizin- 7a(5H)- yl)methoxy) quinazoline | 578.4 (M + H)⁺ | ¹H NMR (400 MHz, METHANOL-d₄) δ ppm 7.70- 7.92 (m, 1 H), 7.54 (s, 1 H), 7.44 (s, 1 H), 5.47-5.72 (m, 1 H), 4.61-4.81 (m, 4 H), 4.20-4.38 (m, 2 H), 3.76-4.14 (m, 5 H), 3.38- 3.58 (m, 1 H), 2.67 (s, 2 H), 2.26- 2.48 (m, 3 H), 2.10-2.25 (m, 8 H), 1.97 (d, J = 1.7 Hz, 3 H). |
| 209 | 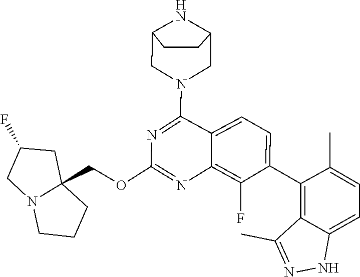 [SMILES: Cc1ccc2[nH]nc(C)c2c1-c1ccc2c(N3CC4CCC(C3)N4)nc(OC[C@@]34CCCN3C[C@H](F)C4)nc2c1F] | 4-((1R,5S)-3,8- diazabicyclo[3.2.1] octan-3-yl)-7- (3,5-dimethyl-1H- indazol-4-yl)-8- fluoro-2- (((2R,7aS)-2- fluorotetrahydro- 1H-pyrrolizin- 7a(5H)- yl)methoxy) quinazoline | 560.4 (M + H)⁺ | ¹H NMR (400 MHz, METHANOL-d₄) δ ppm 7.94- 8.18 (m, 1 H), 7.45-7.60 (m, 1 H), 7.30-7.44 (m, 2 H), 5.49- 5.70 (m, 1 H), 4.66-4.82 (m, 4 H), 4.24-4.42 (m, 2 H), 3.81- 4.12 (m, 5 H), 3.42-3.63 (m, 1 H), 2.51-2.76 (m, 2 H), 2.27-2.45 (m, 3 H), 2.10-2.25 (m, 8H), 1.91 (d, J = 1.3 Hz, 3 H). |

### 4-(4-((1R,5S)-3,&-Diazabicyclo[3.2.1]octan-3-yl)-2-(((2R,7aS)-2-fluorotetrahydro-1H-pyrrolizin-7a(5H)-yl)methoxy)quinolin-7-yl)-5-ethyl-6-fluoronaphthalen-2-ol (Example 210

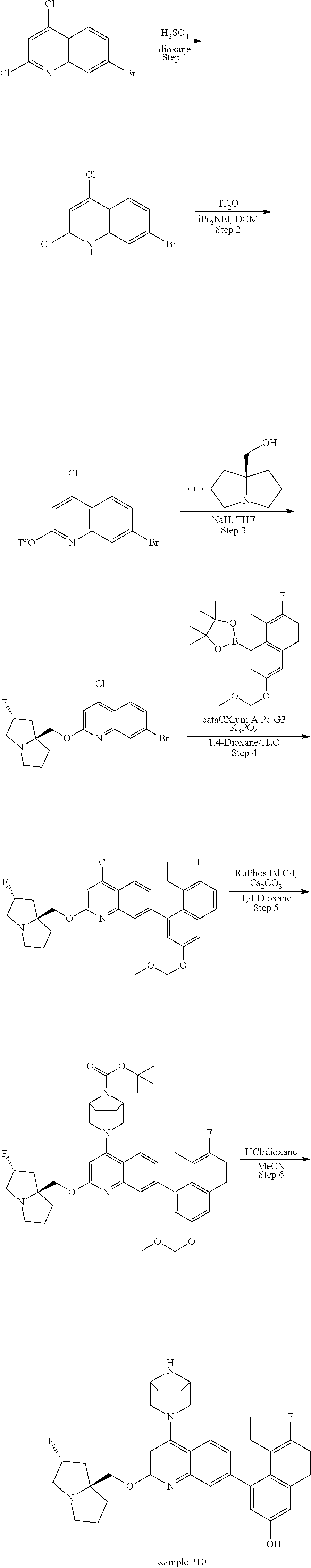

Step 1: 7-Bromo-4-chloroquinolin-2(1H)-one. To a mixture of 7-bromo-2,4-dichloroquinoline (1.00 g, 3.61 mmol) in sulfuric acid (5.88 g, 5.9 mL, 59.9 mmol) diluted to 20% with water was added 1,4-Dioxane (30 mL) and the reaction was stirred at 100° C. for 22 h. After cooling to rt, the reaction mixture was quenched with water. The resulting solid was collected by filtration, washed with water, dried in vacuo to give 7-bromo-4-chloroquinolin-2(1H)-one (0.78 g, 3.02 mmol, 84% yield) as an off-white solid. m/z (ESI, +ve ion): 258.1 (M+H)⁺.

Step 2: 7-Bromo-4-chloroquinolin-2-yl trifluoromethanesulfonate. To a suspension of 7-bromo-4-chloroquinolin-2(1H)-one (0.29 g, 1.13 mmol) in dichloromethane (3.8 mL) was added N,N-dimethyltriethylamine (0.22 mg, 0.3 mL, 1.69 mmol), followed by dropwise addition of trifluoromethanesulfonic anhydride solution (1 M in DCM, 1.2 mL, 1.24 mmol). The reaction mixture was stirred at rt for 1 h. The solvent was evaporated and the residue solid was used directly in the subsequent step. m/z (ESI, +ve ion): 391.6 (M+H)⁺.

Step 3: 7-Bromo-4-chloro-2-(((2R,7aS)-2-fluorotetrahydro-1H-pyrrolizin-7a(5H)-yl)methoxy)quinoline. To the solution of ((2R,7aS)-2-fluorotetrahydro-1H-pyrrolizin-7a(5H)-yl)methanol (0.36 g, 2.23 mmol, PharmaBlock) in THF (2.0 mL) was added sodium hydride (94 mg, 2.34 mmol, TCI America). The reaction mixture was stirred at rt for 15 min. The mixture was then added to a solution of 7-bromo-4-chloroquinolin-2-yl trifluoromethanesulfonate (0.44 g, 1.11 mmol) in THF (2.0 mL). The vial was rinsed with THF (2.0 mL) and the solution was combined into the reaction mixture, which was stirred at 60° C. for 48 h. After cooling to rt, water was added to the reaction mixture and the mixture was extracted with EtOAc (3×). The combined organic layer was dried over anhydrous Na₂SO₄ and concentrated under reduced pressure. The crude material was purified by column chromatography on silica gel, eluting with a gradient of 0-20% 3:1 EtOAc/EtOH in heptane to provide 7-bromo-4-chloro-2-(((2R,7aS)-2-fluorotetrahydro-1H-pyrrolizin-7a(5H)-yl)methoxy)quinoline (80 mg, 0.20 mmol, 18% yield) as an off-white solid. m/z (ESI, +ve ion): 417.1 (M+H)⁺.

Step 4: 4-Chloro-7-(8-ethyl-7-fluoro-3-(methoxymethoxy)naphthalen-1-yl)-2-(((2R,7aS)-2-fluorotetrahydro-1H-pyrrolizin-7a(5H)-yl)methoxy)quinoline. A mixture of 7-bromo-4-chloro-2-(((2R,7aS)-2-fluorotetrahydro-1H-pyrrolizin-7a(5H)-yl)methoxy)quinoline (75 mg, 0.19 mmol), 2-(8-ethyl-7-fluoro-3-(methoxymethoxy)naphthalen-1-yl)-4,4,5,5-tetramethyl-1,3,2-dioxaborolane (0.10 g, 0.28 mmol, LabNetwork), cataCXium A Pd G3 (27 mg, 0.038 mmol, Sigma Aldrich Corporation), and potassium phosphate (0.12 g, 0.56 mmol) in a round-bottom flask was flushed with nitrogen. 1,4-Dioxane (1.6 mL) and water (0.3 mL) were added and the reaction mixture was stirred at 85-90° C. for 3 h. The reaction mixture was concentrated under reduced pressure and the residue was purified by column chromatography on silica gel, eluting with a gradient of 0-25% 3:1 EtOAc/EtOH in heptane to provide 4-chloro-7-(8-ethyl-7-fluoro-3-(methoxymethoxy)naphthalen-1-yl)-2-(((2R,7aS)-2-fluorotetrahydro-1H-pyrrolizin-7a(5H)-yl)methoxy)quinoline (32 mg, 0.058 mmol, 31% yield) as an off-white solid. m/z (ESI, +ve ion): 552.9 (M+H)⁺.

Step 5: tert-Butyl (1R,5S)-3-(7-(8-ethyl-7-fluoro-3-(methoxymethoxy)naphthalen-1-yl)-2-(((2R,7aS)-2-fluorotetrahydro-1H-pyrrolizin-7a(5H)-yl)methoxy)quinolin-4-yl)-3,8-diazabicyclo[3.2.1]octane-8-carboxylate. A mixture of 4-chloro-7-(8-ethyl-7-fluoro-3-(methoxymethoxy)naphthalen-1-yl)-2-(((2R,7aS)-2-fluorotetrahydro-1H-pyrrolizin-7a(5H)-yl)methoxy)quinoline (32 mg, 0.058 mmol), 8-Boc-3,8-diaza-bicyclo[3.2.1]octane (25 mg, 0.11 mmol, Chem-Impex International, Inc.), RuPhos Pd G4 (9.8 mg, 0.012 mmol, Sigma Aldrich Corporation), and cesium carbonate (57 mg, 0.17 mmol) in a round-bottom flask was flushed with nitrogen. 1,4-Dioxane (0.4 mL) was added and the reaction mixture was stirred at 70° C. for 1 h. After cooling to rt, the crude material was concentrated under reduced pressure and the residue was purified by column chromatography on silica gel, eluting with a gradient of 0-80% EtOAc in heptane to provide tert-butyl (1R,5S)-3-(7-(8-ethyl-7-fluoro-3-(methoxymethoxy)naphthalen-1-yl)-2-(((2R,7aS)-2-fluorotetrahydro-1H-pyrrolizin-7a(5H)-yl)methoxy)quinolin-4-yl)-3,8-diazabicyclo[3.2.1]octane-8-carboxylate (16 mg, 0.022 mmol, 38% yield) as white solid. m/z (ESI, +ve ion): 729.0 (M+H)⁺.

Step 6: 4-(4-((1R,5S)-3,8-Diazabicyclo[3.2.1]octan-3-yl)-2-(((2R,7aS)-2-fluorotetrahydro-1H-pyrrolizin-7a(5H)-yl)methoxy)quinolin-7-yl)-5-ethyl-6-fluoronaphthalen-2-ol. To a solution of tert-butyl (1R,5S)-3-(7-(8-ethyl-7-fluoro-3-(methoxymethoxy)naphthalen-1-yl)-2-(((2R,7aS)-2-fluorotetrahydro-1H-pyrrolizin-7a(5H)-yl)methoxy)quinolin-4-yl)-3,8-diazabicyclo[3.2.1]octane-8-carboxylate (10 mg, 0.014 mmol) in acetonitrile (0.2 mL) cooled to 0° C. was added HCl solution (4.0 M in dioxane, 0.4 mL, 0.35 mmol) dropwise. The reaction mixture was stirred at 0° C. for 30 min. The reaction mixture was concentrated and the residual white solid was triturated with diethyl ether (1 mL). The solid was dried under reduced pressure, then dissolved in MeOH and concentrated again. Dichloromethane was added and the mixture was concentrated in vacuo to give 4-(4-((1R,5S)-3,8-diazabicyclo[3.2.1]octan-3-yl)-2-(((2R,7aS)-2-fluorotetrahydro-1H-pyrrolizin-7a(5H)-yl)methoxy)quinolin-7-yl)-5-ethyl-6-fluoronaphthalen-2-ol trihydrochloride as HCl salt (9.6 mg, 0.014 mmol, 100% yield, 95% purity) as a light-yellow solid. m/z (ESI, +ve ion): 585.2 (M+H)⁺. ¹H NMR (METHANOL-d₄, 400 MHz) δ 8.15 (d, 1H, J=8.6 Hz), 7.85 (d, 1H, J=1.5 Hz), 7.5-7.7 (m, 2H), 7.2-7.3 (m, 2H), 6.95 (s, 1H), 6.83 (s, 1H), 5.5-5.7 (m, 1H), 4.32 (br s, 2H), 3.8-4.0 (m, 5H), 3.6-3.8 (m, 3H), 3.5-3.6 (m, 3H), 2.1-2.7 (m, 12H), 0.74 (t, 3H, J=7.3 Hz).

### 4-(4-((1R,5S)-3,8-Diazabicyclo[3.2.1]octan-3-yl)-8-fluoro-2-(((2R,7aS)-2-fluorotetrahydro-1H-pyrrolizin-7a(5H)-yl)methoxy)quinolin-7-yl)-5-ethyl-6-fluoronaphthalen-2-ol (Example 211

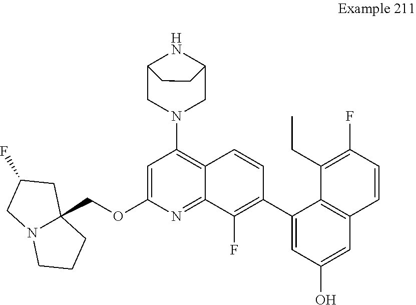 [SMILES: CCc1c(F)ccc2cc(O)cc(-c3ccc4c(N5CC6CCC(C5)N6)cc(OC[C@@]56CCCN5C[C@H](F)C6)nc4c3F)c12]

Synthesized in an analogous manner as Example 210. m/z (ESI, +ve ion): 603.3 (M+H)⁺. ¹H NMR (METHANOL-d₄, 400 MHz) δ 7.98 (br d, 1H, J=8.4 Hz), 7.68 (dd, 1H, J=5.9, 9.0 Hz), 7.56 (br t, 1H, J=6.6 Hz), 7.2-7.3 (m, 2H), 6.8-7.0 (m, 2H), 5.5-5.7 (m, 1H), 4.32 (br s, 2H), 3.6-4.0 (m, 7H), 3.50 (br d, 3H, J=6.5 Hz), 2.2-2.8 (m, 11H), 0.77 (br t, 3H, J=6.6 Hz).

### 5-(((4-((1R,5S)-3,8-Diazabicyclo[3.2.1]octan-3-yl)-7-(8-ethyl-7-fluoro-3-hydroxynaphthalen-1-yl)-6,8-difluoroquinazolin-2-yl)oxy)methyl)-5-methyldihydrofuran-2(3H)-one (Example 212

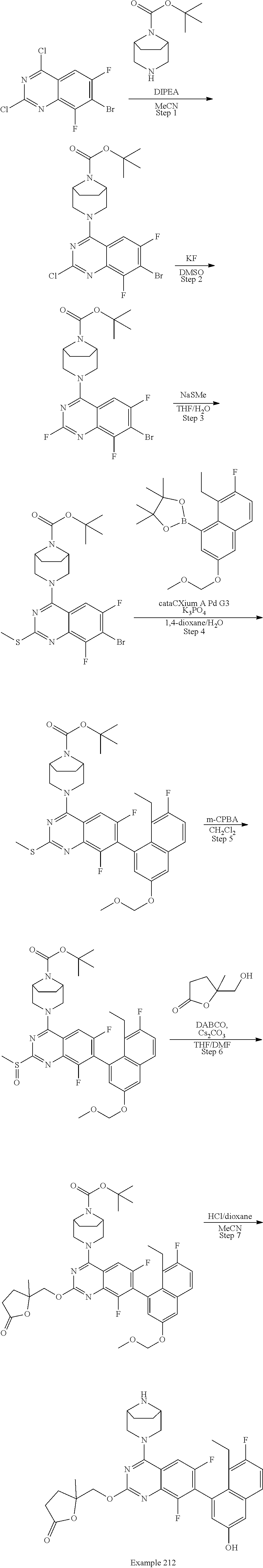

Step 1: tert-Butyl (1R,5S)-3-(7-bromo-2-chloro-6,8-difluoroquinazolin-4-yl)-3,8-diazabicyclo[3.2.1]octane-8-carboxylate. A mixture of 7-bromo-2,4-dichloro-6,8-difluoroquinazoline (2.37 g, 7.54 mmol), tert-butyl 3,84diazabicyclo[3.2.1]octane-8-carboxylate (1.70 g, 7.92 mmol), and DIPEA (2.90 g, 3.4 mL, 22.6 mmol,) in acetonitrile (35 mL) was stirred at rt for 1 h. The precipitate was collected by filtration and washed with heptane. The filtrate was diluted with water and extracted with DCM. The organic layer was dried over sodium sulfate and evaporated in vacuo to give tert-butyl (1R,5S)-3-(7-bromo-2-chloro-6,8-difluoroquinazolin-4-yl)-3,8-diazabicyclo[3.2.1]octane-8-carboxylate (3.70 g, 7.62 mmol, 101% yield) as a light yellow solid, which was used directly for next step without further purification. m/z (ESI, +ve ion): 490.0 (M+H)⁺.

Step 2: tert-Butyl (1R,5S)-3-(7-bromo-2,6,8-trifluoroquinazolin-4-yl)-3,8-diazabicyclo[3.2.1]octane-8-carboxylate. A mixture of tert-butyl (1R,5S)-3-(7-bromo-2-chloro-6,8-difluoroquinazolin-4-yl)-3,8-diazabicyclo[3.2.]octane-8-carboxylate (0.80 g, 1.63 mmol) and anhydrous potassium fluoride (0.14 g, 2.45 mmol) in dimethyl sulfoxide (6.0 mL) was stirred at 120° C. overnight. The reaction mixture was diluted with water and extracted with DCM. The organic layer was dried over Na₂SO₄ and evaporated in vacuo to afford tert-butyl (1R,5S)-3-(7-bromo-2,6,8-trifluoroquinazolin-4-yl)-3,8-diazabicyclo[3.2.1]octane-8-carboxylate (0.91 g), which was used in the next step without further purification. m/z (ESI, +ve ion): 473.0 (M+H)⁺.

Step 3: tert-Butyl (1R,5S)-3-(7-bromo-6,8-difluoro-2-(methylthio)quinazolin-4-yl)-3,8-diazabicyclo[3.2.1]octane-8-carboxylate. To a stirring solution of tert-butyl (1R,5S)-3-(7-bromo-2,6,8-trifluoroquinazolin-4-yl)-3,8-diazabicyclo[3.2.1]octane-8-carboxylate (0.91 g, 1.91 mmol) in tetrahydrofuran (8.0 mL) at 0° C. was added sodium methanethiolate (0.13 g, 1.91 mmol) in water (2.0 mL). The reaction was then stirred at rt for 4 h. The reaction mixture was diluted with water and extracted with DCM. The organic layer was concentrated under reduced pressure and purified by column chromatography on silica gel, eluting with a gradient of 0-20% EtOAc in heptane to afford tert-butyl (1R,5S)-3-(7-bromo-6,8-difluoro-2-(methylthio)quinazolin-4-yl)-3,8-diazabicyclo[3.2.1]octane-8-carboxylate (0.44 g, 0.88 mmol, 46% yield) as a yellow solid. m/z (ESI, +ve ion): 501.0 (M+H)⁺.

Step 4: tert-Butyl (1R,5S)-3-(7-(8-ethyl-7-fluoro-3-(methoxymethoxy)naphthalen-1-yl)-6,8-difluoro-2-(methylthio)quinazolin-4-yl)-3,8-diazabicyclo[3.2.1]octane-8-carboxylate. In a vial, the mixture of tert-butyl (1R,5S)-3-(7-bromo-6,8-difluoro-2-(methylthio)quinazolin-4-yl)-3,8-diazabicyclo[3.2.1]octane-8-carboxylate (0.44 g, 0.88 mmol), 2-(8-ethyl-7-fluoro-3-(methoxymethoxy)naphthalen-1-yl)-4,4,5,5-tetramethyl-1,3,2-dioxaborolane (0.48 g, 1.33 mmol, LabNetwork), cataCXium A Pd G3 (0.13 g, 0.18 mmol, Sigma Aldrich Corporation), and potassium phosphate tribasic (0.56 g, 2.65 mmol) was suspended in degassed water (0.8 mL) and 1,4-dioxane (4.0 mL). The reaction was stirred at 90° C. for 1.5 h. The reaction mixture was concentrated and purified by column chromatography on silica gel, eluting with a gradient of 0-50% 3:1 EtOAc/EtOH in heptane with 2% triethylamine additive to afford tert-butyl (1R,5S)-3-(7-(8-ethyl-7-fluoro-3-(methoxymethoxy)naphthalen-1-yl)-6,8-difluoro-2-(methylthio)quinazolin-4-yl)-3,8-diazabicyclo[3.2.1]octane-8-carboxylate (0.46 g, 0.70 mmol, 79% yield) as a white solid. m/z (ESI, +ve ion): 655.2 (M+H)⁺.

Step 5: tert-Butyl (1R,5S)-3-(7-(8-ethyl-7-fluoro-3-(methoxymethoxy)naphthalen-1-yl)-6,8-difluoro-2-(methylsulfinyl)quinazolin-4-yl)-3,8-diazabicyclo[3.2.1]octane-8-carboxylate. To a solution of tert-butyl (1R,5S)-3-(7-(8-ethyl-7-fluoro-3-(methoxymethoxy)naphthalen-1-yl)-6,8-difluoro-2-(methylthio)quinazolin-4-yl)-3,8-diazabicyclo[3.2.1]octane-8-carboxylate (0.46 mg, 0.70 mmol) in dichloromethane (3 mL) at 0° C. was added 3-chloroperoxybenzoic acid (0.13 mg, 0.77 mmol) in dichloromethane (1.0 mL) slowly. The reaction mixture was stirred at 0° C. for 1 h. The reaction mixture was purified by column chromatography on silica gel, eluting with a gradient of 0-50% 3:1 EtOAc/EtOH blend in heptane with 2% triethylamine additive to afford tert-butyl (1R,5S)-3-(7-(8-ethyl-7-fluoro-3-(methoxymethoxy)naphthalen-1-yl)-6,8-difluoro-2-(methylsulfinyl)quinazolin-4-yl)-3,8-diazabicyclo[3.2.1]octane-8-carboxylate (0.38 g, 0.57 mmol, 81% yield) as a yellow solid. m/z (ESI, +ve ion): 671.2 (M+H)⁺. ¹H NMR (400 MHz, METHANOL-d₄) δ ppm 7.79-7.85 (m, 2H), 7.65 (d, J=2.7 Hz, 1H), 7.34 (t, J=9.3 Hz, 1H), 7.19 (t, J=2.6 Hz, 1H), 5.35 (s, 2H), 4.60-4.79 (m, 2H), 4.43 (br s, 2H), 3.69-3.82 (m, 2H), 3.53 (s, 3H), 3.02 (d, J=2.5 Hz, 3H), 2.55-2.65 (m, 1H), 2.40-2.51 (m, 1H), 1.78-2.03 (m, 4H), 1.55 (s, 9H), 0.76-0.88 (m, 3H).

Step 6: tert-Butyl (1R,5S)-3-(7-(8-ethyl-7-fluoro-3-(methoxymethoxy)naphthalen-1-yl)-6,8-difluoro-2-((2-methyl-5-oxotetrahydrofuran-2-yl)methoxy)quinazolin-4-yl)-3,8-diazabicyclo[3.2.1]octane-8-carboxylate. A mixture of tert-butyl (1R,5S)-3-(7-(8-ethyl-7-fluoro-3-(methoxymethoxy)naphthalen-1-yl)-6,8-difluoro-2-(methylsulfinyl)quinazolin-4-yl)-3,8-diazabicyclo[3.2.1]octane-8-carboxylate (20 mg, 0.03 mmol), 5-(hydroxymethyl)-5-methyldihydrofuran-2(3H)-one (3.9 mg, 0.03 mmol), cesium carbonate (29 mg, 0.09 mmol), and 1,4-diazabicyclo[2.2.2]octane (0.7 mg, 5.96 μmol) in tetrahydrofuran (1.0 mL) and N,N-dimethylformamide (0.3 mL) was stirred at rt for 1 h. Volatiles were removed in vacuo and the crude residue was purified by column chromatography on silica gel, eluting with a gradient of 0-18% MeOH in DCM to give tert-butyl (1R,5S)-3-(7-(8-ethyl-7-fluoro-3-(methoxymethoxy)naphthalen-1-yl)-6,8-difluoro-2-((2-methyl-5-oxotetrahydrofuran-2-yl)methoxy)quinazolin-4-yl)-3,8-diazabicyclo[3.2.1]octane-8-carboxylate, which was used in the subsequent step directly. m/z (ESI, +ve ion): 737.2 (M+1)⁺.

Step 7: 5-(((4-((1R,5S)-3,8-Diazabicyclo[3.2.1]octan-3-yl)-7-(8-ethyl-7-fluoro-3-hydroxynaphthalen-1-yl)-6,8-difluoroquinazolin-2-yl)oxy)methyl)-5-methyldihydrofuran-2(3H)-one. To a solution of the above tert-butyl (1R,5S)-3-(7-(8-ethyl-7-fluoro-3-(methoxymethoxy)naphthalen-1-yl)-6,8-difluoro-2-((2-methyl-5-oxotetrahydrofuran-2-yl)methoxy)quinazolin-4-yl)-3,8-diazabicyclo[3.2.1]octane-8-carboxylate in MeCN (2.0 mL) was added HCl solution (4.0 M in dioxane, 0.2 mL, 0.95 mmol) at rt. The resulting mixture was stirred at rt for 1 h. Volatiles were removed under reduced pressure. The crude residue was purified by reverse-phase HPLC to give 5-(((4-((1R,5S)-3,8-diazabicyclo[3.2.1]octan-3-yl)-7-(8-ethyl-7-fluoro-3-hydroxynaphthalen-1-yl)-6,8-difluoroquinazolin-2-yl)oxy)methyl)-5-methyldihydrofuran-2(3H)-one as a TFA salt and white solid (2.5 mg, 0.004 mmol, 16% combined yield over 2 steps). m/z (ESI, +ve ion): 593.2 (M+H)⁺. ¹H NMR (METHANOL-d₄, 400 MHz) δ 7.6-7.7 (m, 2H), 7.31 (d, 1H, J=2.7 Hz), 7.25 (t, 1H, J=9.4 Hz), 6.98 (d, 1H, J=2.7 Hz), 4.5-4.7 (m, 4H), 4.25 (br s, 2H), 3.7-3.9 (m, 2H), 2.8-2.9 (m, 1H), 2.4-2.7 (m, 4H), 2.1-2.3 (m, 5H), 1.54 (s, 3H), 0.7-0.9 (m, 3H).

TABLE 8
Additional Examples. Prepared in an Analogous Manner to Example 212.
Products were isolated as corresponding TFA salts.

| | | | MS | |
|---|---|---|---|---|
| | | | m/z | |
| | | | (ESI, | |
| Ex. # | Structure | Name | +ve ion) | ¹H NMR |
| 213 | 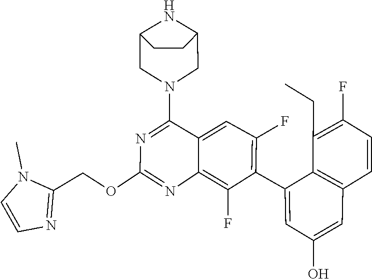 [SMILES: CCc1c(F)ccc2cc(O)cc(-c3c(F)cc4c(N5CC6CCC(C5)N6)nc(OCc5nccn5C)nc4c3F)c12] | 4-(4-((1R,5S)-3,8- diazabicyclo[3.2.1] octan-3-yl)-6,8- difluoro-2-((1- methyl-1H- imidazol-2- yl)methoxy) quinazolin-7- yl)-5-ethyl-6- fluoronaphthalen- 2-ol | 575.2 (M + H)⁺ | ¹H NMR (METHANOL-d₄, 400 MHz) δ 7.6-7.7 (m, 2 H), 7.52 (d, 1 H, J = 2.1 Hz), 7.49 (d, 1 H, J = 1.9 Hz), 7.31 (d, 1 H, J = 2.5 Hz), 7.26 (t, 1 H, J = 9.3 Hz), 6.95 (d, 1 H, J = 2.7 Hz), 5.6-5.8 (m, 2 H), 4.5-4.7 (m, 2 H), 4.25 (br d, 2H, J = 9.0 Hz), 4.05 (s, 3 H), 3.7- 3.9 (m, 2 H), 2.5-2.6 (m, 1 H), 2.3- 2.4 (m, 1 H), 2.1-2.3 (m, 4 H), 0.75 (t, 3 H, J = 7.4 Hz). |
| 214 | 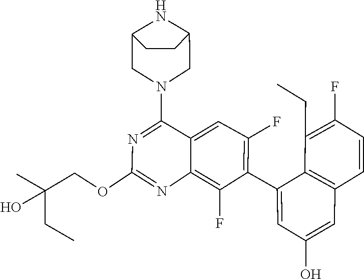 [SMILES: CCc1c(F)ccc2cc(O)cc(-c3c(F)cc4c(N5CC6CCC(C5)N6)nc(OCC(C)(O)CC)nc4c3F)c12] | 4-(4-((1R,5S)-3,8- diazabicyclo[3.2.1] octan-3-yl)-6,8- difluoro-2-(2- hydroxy-2- methylbutoxy) quinazolin-7- yl)-5-ethyl-6- fluoronaphthalen- 2-ol | 567.2 (M + H)⁺ | ¹H NMR (400 MHz, METHANOL-d₄) δ ppm 7.66- 7.73 (m, 2 H), 7.33 (d, J = 2.7 Hz, 1 H), 7.28 (t, J = 9.3 Hz, 1 H), 7.00 (d, J = 2.5 Hz, 1 H), 4.55-4.74 (m, 2 H), 4.37 (s, 2 H), 4.28 (br s, 2 H), 3.78-3.92 (m, 2 H), 2.59 (td, J = 7.1, 2.1 Hz, 1 H), 2.44 (br d, J = 2.7 Hz, 1 H), 2.14-2.31 (m, 4 H), 1.70 (q, J = 7.4 Hz, 2 H), 1.31 (s, 3 H), 1.00 (t, J = 7.5 Hz, 3 H), 0.83 (t, J = 7.3 Hz, 3 H). |
| 216 | 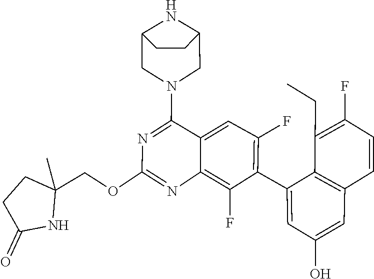 [SMILES: CCc1c(F)ccc2cc(O)cc(-c3c(F)cc4c(N5CC6CCC(C5)N6)nc(OCC5(C)CCC(=O)N5)nc4c3F)c12] | 5-(((4-((1R,5S)- 3,8- diazabicyclo[3.2.1] octan-3-yl)-7-(8- ethyl-7-fluoro-3- hydroxynaphthalen- 1-yl)-6,8- difluoroquinazolin- 2-yl)oxy)methyl)- 5- methylpyrrolidin- 2-one | 592.2 (M + H)⁺ | ¹H NMR (METHANOL-d₄, 400 MHz) δ 7.66 (dd, 1 H, J = 5.9, 9.0 Hz), 7.60 (dd, 1 H, J = 1.7, 10.0 Hz), 7.29 (d, 1 H, J = 2.7 Hz), 7.23 (t, 1 H, J = 9.3 Hz), 6.98 (d, 1 H, J = 2.7 Hz), 4.4-4.5 (m, 3 H), 4.3- 4.4 (m, 1 H), 3.5-3.7 (m, 4 H), 2.5- 2.7 (m, 2H), 2.3-2.5 (m, 2 H), 2.2- 2.3 (m, 1H), 1.9-2.0 (m, 1 H), 1.85 (s, 4 H), 1.41 (s, 3 H), 0.81 (t, 3 H, J = 7.4 Hz). |
| 217 | 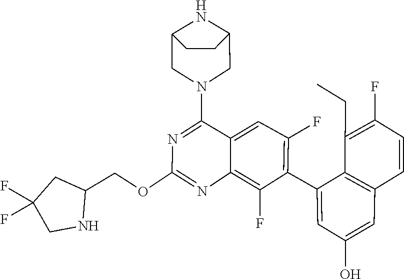 [SMILES: CCc1c(F)ccc2cc(O)cc(-c3c(F)cc4c(N5CC6CCC(C5)N6)nc(OCC5CC(F)(F)CN5)nc4c3F)c12] | 4-(4-((1R,5S)-3,8- diazabicyclo[3.2.1] octan-3-yl)-2- (((S)-4,4- difluoropyrrolidin- 2-yl)methoxy)- 6,8- difluoroquinazolin- 7-yl)-5-ethyl-6- fluoronaphthalen- 2-ol | 600.2 (M + H)⁺ | ¹H NMR (400 MHz, METHANOL-d₄) δ ppm 7.68- 7.74 (m, 2 H), 7.33 (d, J = 2.7 Hz, 1 H), 7.28 (t, J = 9.4 Hz, 1 H), 7.00 (d, J = 2.7 Hz, 1 H), 4.81-4.94 (m, 1 H), 4.60-4.74 (m, 3 H), 4.44- 4.55 (m, 1 H), 4.28 (br s, 2 H), 3.78-3.98 (m, 4 H), 2.87 (dq, J = 14.7, 7.4 Hz, 1 H), 2.53-2.73 (m, 2 H), 2.38-2.51 (m, 1 H), 2.14-2.27 (m, 4 H), 0.79-0.86 (m, 3 H). |

### 4-(4-((1R,5S)-3,8-Diazabicyclo[3.2.1]octan-3-yl)-6,8-difluoro-2-((E)-3-hydroxy-3-methylbut-1-en-1-yl)quinazolin-7-yl)-S-ethyl-6-fluoronaphthalen-2-ol (Example 218

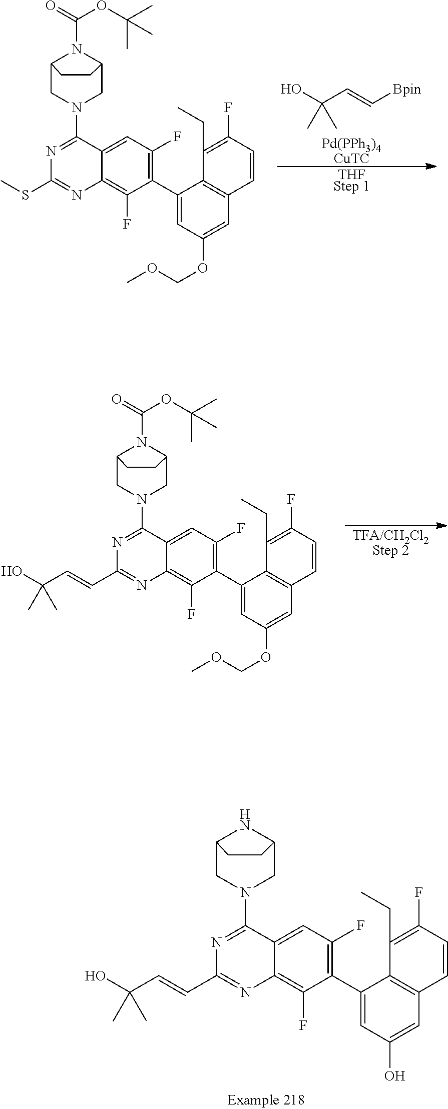

Step 1: tert-Butyl (1R,5S)-3-(7-(8-ethyl-7-fluoro-3-(methoxymethoxy)naphthalen-1-yl)-6,8-difluoro-2-((E)-3-hydroxy-3-methylbut-1-en-1-yl)quinazolin-4-yl)-3,8-diazabicyclo[3.2.1]octane-8-carboxylate. A 5-mL cone-shaped microwave reaction vessel was charged with tert-butyl (1R,5S)-3-(7-(8-ethyl-7-fluoro-3-(methoxymethoxy)naphthalen-1-yl)-6,8-difluoro-2-(methylthio)quinazolin-4-yl)-3,8-diazabicyclo[3.2.1]octane-8-carboxylate (48 mg, 0.07 mmol), (3E)-2-methyl-4-(4,4,5,5-tetramethyl-1,3,2-dioxaborolan-2-yl)but-3-en-2-ol (31 mg, 0.15 mmol), tetrakis(triphenylphosphine)palladium(0) (8.5 mg, 7.33 μmol, Strem), copper(I) thiophene-2-carboxylate (28 mg, 0.15 mmol, CombiBlocks) and THF (4.0 mL). The resulting mixture was purged with nitrogen for 5 min before sealed and irradiated under microwave at 90° C. for 1.5 h. Volatiles were removed under reduced pressure. The crude residue was purified by column chromatography on silica gel, eluting with a gradient of 0-90% MeOH in DCM to give tert-butyl (1R,5S)-3-(7-(8-ethyl-7-fluoro-3-(methoxymethoxy)naphthalen-1-yl)-6,8-difluoro-2-((E)-3-hydroxy-3-methylbut-1-en-1-yl)quinazolin-4-yl)-3,8-diazabicyclo[3.2.1]octane-8-carboxylate as an off-white solid, which was used in the next step without further purification. m/z (ESI, +ve ion): 693.2 (M+H)⁺.

Step 2: 4-(4-((1R,5S)-3,8-Diazabicyclo[3.2.1]octan-3-yl)-6,8-difluoro-2-((E)-3-hydroxy-3-methylbut-1-en-1-yl)quinazolin-7-yl)-5-ethyl-6-fluoronaphthalen-2-ol. To a stirring solution of the above tert-butyl (1R,5S)-3-(7-(8-ethyl-7-fluoro-3-(methoxymethoxy)naphthalen-1-yl)-6,8-difluoro-2-((E)-3-hydroxy-3-methylbut-1-en-1-yl)quinazolin-4-yl)-3,8-diazabicyclo[3.2.1]octane-8-carboxylate in DCM (2.0 mL) was added TFA (1.50 g, 1.0 mL, 13.1 mmol) at rt. The resulting mixture was stirred at rt for 0.5 h. Volatiles were removed under reduced pressure. The crude residue was dissolved in MeOH/DCM and cooled in an ice bath before ammonium hydroxide (1.0 mL) was added to neutralize. The resulting mixture was purified by column chromatography on silica gel, eluting with a gradient of 0-100% MeOH in DCM with 0.5% ammonium hydroxide to give 4-(4-((1R,5S)-3,8-diazabicyclo[3.2.1]octan-3-yl)-6,8-difluoro-2-((E)-3-hydroxy-3-methylbut-1-en-1-yl)quinazolin-7-yl)-5-ethyl-6-fluoronaphthalen-2-ol (5 mg, 9.11 μmol, 13% combined yield over 2 steps) as a white solid. m/z (ESI, +ve ion): 549.2 (M+H)⁺. ¹H NMR (METHANOL-d₄, 400 MHz) δ 7.68 (dd, 1H, J=6.0, 9.1 Hz), 7.61 (dd, 1H, J=1.8, 9.9 Hz), 7.33 (d, 1H, J=15.7 Hz), 7.30 (d, 1H, J=2.7 Hz), 7.24 (t, 1H, J=9.4 Hz), 7.01 (d, 1H, J=2.5 Hz), 6.69 (d, 1H, J=15.5 Hz), 4.4-4.5 (m, 2H), 3.5-3.7 (m, 4H), 2.3-2.7 (m, 2H), 1.87 (br s, 4H), 1.43 (s, 6H), 0.81 (t, 3H, J=7.3 Hz).

### 4-((1R,5S)-3,8-Diazabicyclo[3.2.1]octan-3-yl)-8-fluoro-2-(((2R,7aS)-2-fluorotetrahydro-1H-pyrrolizin-7a(5H)-yl)methoxy)-7-(3-hydroxynaphthalen-1-yl)quinazoline-6-carbonitrile (Example 219

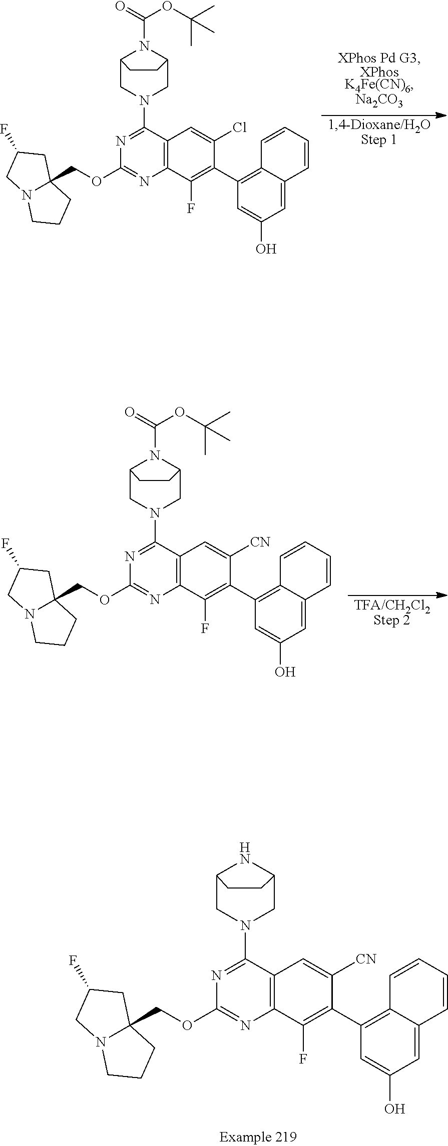 [SMILES: CC(C)(C)OC(=O)N1C2CCC1CN(c1nc(OC[C@@]34CCCN3C[C@H](F)C4)nc3c(F)c(-c4cc(O)cc5ccccc45)c(Cl)cc13)C2 ; "1,4-Dioxane/H2OStep 1" ; "XPhos Pd G3,XPhosK4Fe(CN)6,Na2CO3" ; CC(C)(C)OC(=O)N1C2CCC1CN(c1nc(OC[C@@]34CCCN3C[C@H](F)C4)nc3c(F)c(-c4cc(O)cc5ccccc45)c(C#N)cc13)C2 ; "TFA/CH2Cl2Step 2" ; N#Cc1cc2c(N3CC4CCC(C3)N4)nc(OC[C@@]34CCCN3C[C@H](F)C4)nc2c(F)c1-c1cc(O)cc2ccccc12]

Step 1: tert-Butyl (1R,5S)-3-(6-cyano-8-fluoro-2-(((2R,7aS)-2-fluorotetrahydro-1H-pyrrolizin-7a(5H)-yl)methoxy)-7-(3-hydroxynaphthalen-1-yl)quinazolin-4-yl)-3,8-diazabicyclo[3.2.1]octane-8-carboxylate. To a vial was added tert-butyl (1R,5S)-3-(6-chloro-8-fluoro-2-(((2R,7aS)-2-fluorotetrahydro-1H-pyrrolizin-7a(5H)-yl)methoxy)-7-(3-hydroxynaphthalen-1-yl)quinazolin-4-yl)-3,8-diazabicyclo[3.2.1]octane-8-carboxylate (86 mg, 0.12 mmol, synthesized in an analogous manner to Example 109), potassium ferrocyanide trihydrate (26 mg, 0.06 mmol), Na₂CO₃ (1.6 mg, 0.02 mmol), and 2-dicyclohexylphosphino-2′,4′,6′,-triisopropylbiphenyl (12 mg, 0.03 mmol, Sigma Aldrich Corporation) in dioxane (0.6 mL) and water (0.6 mL). (2-Dicyclohexylphosphino-2′,4′,6′-triisopropyl-1,1′-biphenyl)[2-(2′-amino-1,1′-biphenyl)]palladium(II) methanesulfonate (11 mg, 0.01 mmol, Sigma Aldrich Corporation) was then added. The vial was purged with Ar, and the reaction was stirred at 90° C. for 1 h. The reaction was diluted with aqueous NaHCO₃ (5 mL), extracted with EtOAc (3×5 mL), dried over MgSO₄, filtered and concentrated under reduced pressure. The crude material was purified by column chromatography on silica gel, eluting with a gradient of 0-100% 3:1 EtOAc/EtOH in heptane to provide tert-butyl (1R,5S)-3-(6-cyano-8-fluoro-2-(((2R,7aS)-2-fluorotetrahydro-1H-pyrrolizin-7a(5H)-yl)methoxy)-7-(3-hydroxynaphthalen-1-yl)quinazolin-4-yl)-3,8-diazabicyclo[3.2.1]octane-8-carboxylate (72 mg, 0.11 mmol, 84% yield) as a pale brown solid. m/z (ESI, +ve ion): 683.4 (M+H)⁺.

Step 2: 4-((1R,5S)-3,8-Diazabicyclo[3.2.1]octan-3-yl)-8-fluoro-2-(((2R,7aS)-2-fluorotetrahydro-1H-pyrrolizin-7a(5H)-yl)methoxy)-7-(3-hydroxynaphthalen-1-yl)quinazoline-6-carbonitrile. To a vial was added tert-butyl (1R,5S)-3-(6-cyano-8-fluoro-2-(((2R,7aS)-2-fluorotetrahydro-1H-pyrrolizin-7a(5H)-yl)methoxy)-7-(3-hydroxynaphthalen-1-yl)quinazolin-4-yl)-3,8-diazabicyclo[3.2.1]octane-8-carboxylate (72 mg, 0.11 mmol) and TFA (0.24 mg, 0.2 mL, 2.09 mmol) in dichloromethane (1.0 mL). The reaction was stirred at 35° C. for 1 h. The reaction was concentrated under reduced pressure. The crude product was purified by reverse phase HPLC. The acetonitrile/water solution of product was diluted with sat. NaHCO₃ (10 mL), extracted with EtOAc (3×10 mL), dried over MgSO₄, filtered and concentrated in vacuo to give 4-((1R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)-7-(3-hydroxynaphthalen-1-yl)quinazoline-6-carbonitrile (41 mg, 0.07 mmol, 67% yield) as a tan solid. m/z (ESI, +ve ion): 583.2 (M+H)⁺. ¹H NMR (400 MHz, DMSO-d₆) δ ppm 9.84-10.35 (m, 1H), 8.38 (s, 1H), 7.84 (d, J=8.4 Hz, 1H), 7.47 (ddd, J=8.1, 6.5, 1.5 Hz, 1H), 7.33 (d, J=2.3 Hz, 1H), 7.23-7.32 (m, 2H), 7.17 (dd, J=2.3, 0.8 Hz, 1H), 5.18-5.39 (m, 1H), 4.34-4.48 (m, 2H), 4.13 (dd, J=10.5, 4.2 Hz, 1H), 4.04 (dd, J=10.2, 3.3 Hz, 1H), 3.51-3.70 (m, 4H), 2.97-3.15 (m, 4H), 2.78-2.90 (m, 1H), 1.98-2.19 (m, 3H), 1.74-1.90 (m, 3H), 1.59-1.70 (m, 4H).

### 1-(4-((1R,5S)-3,8-diazabicyclo[3.2.1]octan-3-yl-8-fluoro-2-(((2R,7aS)-2-fluorotetrahydro-1H-pyrrolizin-7a(5H)-yl)methoxy)quinazolin-7-yl)isoquinolin-3-amine Example 220

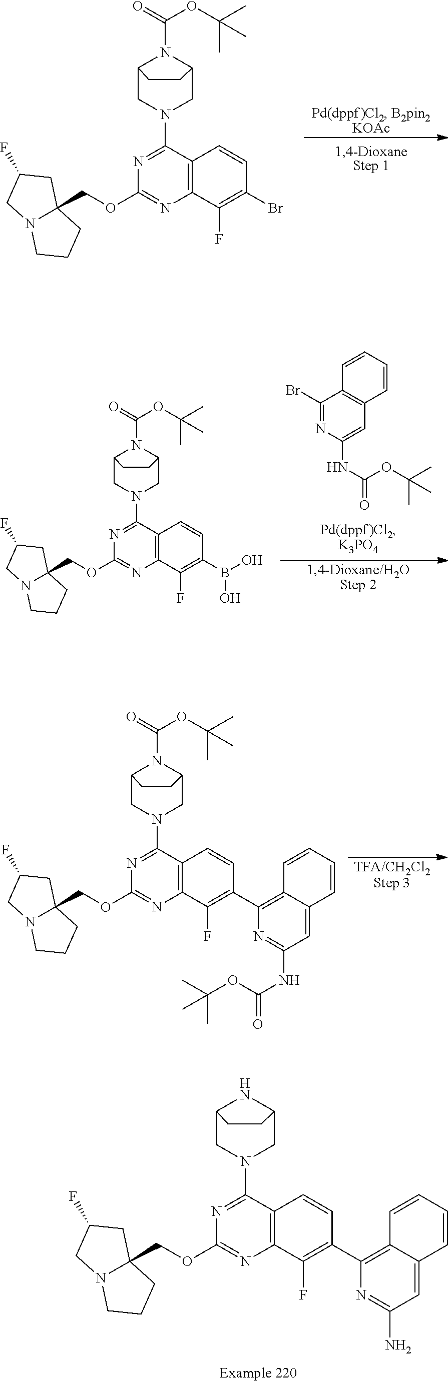

Step 1: (4-((1R,5S)-8-(tert-Butoxycarbonyl)-3,8-diazabicyclo[3.2.1]octan-3-yl)-8-fluoro-2-(((2R,7aS)-2-fluorotetrahydro-1H-pyrrolizin-7a(5H)-yl)methoxy)quinazolin-7-yl)boronic acid. A 20-mL vial was charged with tert-butyl (1R,5S)-3-(7-bromo-8-fluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate (0.50 g, 0.84 mmol), [1,1′-bis(diphenylphosphino)ferrocene]dichloropalladium(II) (0.12 g, 0.17 mmol), potassium acetate (0.25 mg, 2.52 mmol) and bis(pinacalato)diboron (0.27 g, 1.05 mmol) in 1,4-dioxane (4.2 mL). The reaction was purged with nitrogen for 5 min and then stirred at 80° C. overnight. The reaction was then filtered through Celite and the plug was washed with EtOAc. The filtrate was then concentrated under reduced pressure. The crude material (0.72 g) was used in the subsequence steps without further purification. m/z (ESI, +ve ion): 560.2 (M+H⁺).

Step 2: tert-Butyl (1R,5S)-3-(7-(3-((tert-butoxycarbonyl)amino)isoquinolin-1-yl)-8-fluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate. A mixture of (4-((1R,5S)-8-(tert-butoxycarbonyl)-3,8-diazabicyclo[3.2.1]octan-3-yl)-8-fluoro-2-(((2R,7aS)-2-fluorotetrahydro-1H-pyrrolizin-7a(5H)-yl)methoxy)quinazolin-7-yl)boronic acid (0.05 g, 0.089 mmol), tert-butyl (1-bromoisoquinolin-3-yl)carbamate (0.040 g, 0.125 mmol), potassium phosphate tribasic (0.057 g, 0.27 mmol), and [1,1′-bis(diphenylphosphino)ferrocene]dichloropalladium(II) (0.013 g, 0.018 mmol, Sigma Aldrich Corporation) in dioxane (2 mL) and water (1 mL) contained in a vial was purged with Ar, capped and stirred at 90° C. for 3 h. The reaction was then diluted with water and extracted with EtOAc. The organic phase was concentrated under reduced pressure and the residue was purified by column chromatography on silica gel, eluting with 0-100% EtOAc/EtOH (3:1) in heptane to afford tert-butyl (1R,5S)-3-(7-(3-((tert-butoxycarbonyl)amino)isoquinolin-1-yl)-8-fluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate (27 mg, 0.036 mmol, 40% yield). m/z (ESI, +ve ion): 758.6 (M+H)⁺.

Step 3: 1-(4-((1R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)quinazolin-7-yl)isoquinolin-3-amine. A mixture of tert-Butyl (1R,5S)-3-(7-(3-((tert-butoxycarbonyl)amino)isoquinolin-1-yl)-8-fluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate (22 mg, 0.029 mmol) and TFA (0.099 g, 0.07 mL, 0.871 mmol) in DCM (2 mL) was stirred at rt for 2 h. The mixture was concentrated under reduced pressure. The resulting crude residue was purified by reverse phase HPLC to afford 1-(4-((1R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)quinazolin-7-yl)isoquinolin-3-amine tris(2,2,2-trifluoroacetate) (13 mg, 0.014 mmol, 50% yield). m/z (ESI, +ve ion): 558.3 (M+H⁺). ¹H NMR (400 MHz, DMSO-d₆) δ ppm 10.92-11.14 (m, 1H), 9.35-9.54 (m, 1H), 9.09-9.29 (m, 1H), 7.92-8.03 (m, 1H), 7.62-7.70 (m, 1H), 7.29-7.54 (m, 4H), 7.08-7.20 (m, 2H), 6.80 (s, 2H), 5.49-5.71 (m, 2H), 4.61 (s, 6H), 4.16-4.28 (m, 3H), 3.68-3.97 (m, 7H), 3.21-3.43 (m, 1H), 2.11-2.40 (m, 4H).

TABLE 9
Additional Examples. Prepared in an Analogous Manner to Example 220.
Products were isoalted as corresponding TFA salts.

| | | | MS | |
|---|---|---|---|---|
| | | | m/z | |
| | | | (ESI, | |
| Ex. # | Structure | Name | +ve ion) | ¹H NMR |
| 221 | 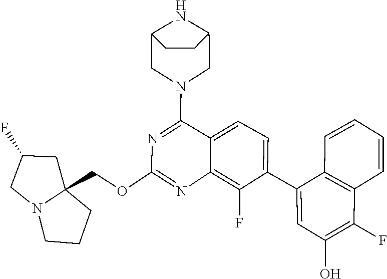 [SMILES: Oc1cc(-c2ccc3c(N4CC5CCC(C4)N5)nc(OC[C@@]45CCCN4C[C@H](F)C5)nc3c2F)c2ccccc2c1F] | 4-(4-((1R,5S)-3,8- diazabicyclo[3.2.1] octan-3-yl)-8- fluoro-2- (((2R,7aS)-2- fluorotetrahydro- 1H-pyrrolizin- 7a(5H)- yl)methoxy) quinazolin-7- yl)-1- fluoronaphthalen- 2-ol | 576.2 (M + H)⁺ | ¹H NMR (METHANOL-d₄, 400 MHz) δ 8.02 (d, 1 H, J = 8.4 Hz), 7.84 (dd, 1 H, J = 0.8, 8.8 Hz), 7.5- 7.6 (m, 2 H), 7.2-7.3 (m, 3 H), 5.2- 5.4 (m, 1 H), 4.4-4.6 (m, 2 H), 4.2- 4.3 (m, 2 H), 3.62 (br s, 4 H), 3.1- 3.3 (m, 3 H), 3.0-3.1 (m, 1 H), 2.1- 2.4 (m, 3 H), 2.0-2.1 (m, 2 H), 1.8- 1.9 (m, 5 H). |
| 222 | 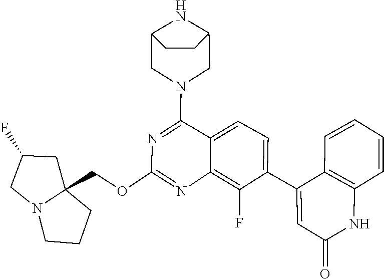 [SMILES: O=c1cc(-c2ccc3c(N4CC5CCC(C4)N5)nc(OC[C@@]45CCCN4C[C@H](F)C5)nc3c2F)c2ccccc2[nH]1] | 4-(4-((1R,5S)-3,8- diazabicyclo[3.2.1] octan-3-yl)-8- fluoro-2- (((2R,7aS)-2- fluorotetrahydro- 1H-pyrrolizin- 7a(5H)- yl)methoxy) quinazolin-7- yl)quinolin- 2-ol | 559.2 (M + H)⁺ | ¹H NMR (METHANOL-d₄, 400 MHz) δ 7.9-8.1 (m, 1 H), 7.6-7.7 (m, 1 H), 7.49 (d, 1 H, J = 7.7 Hz), 7.43 (dd, 1 H, J = 6.7, 8.8 Hz), 7.3- 7.3 (m, 1 H), 7.2-7.2 (m, 1 H), 6.66 (s, 1 H), 5.4-5.7 (m, 1 H), 4.6-4.8 (m, 4 H), 4.26 (br s, 2 H), 3.8-4.1 (m, 5 H), 3.4-3.5 (m, 1 H), 2.5-2.8 (m, 2 H), 2.3-2.5 (m, 3 H), 2.17 (s, 5 H). |
| 223 | 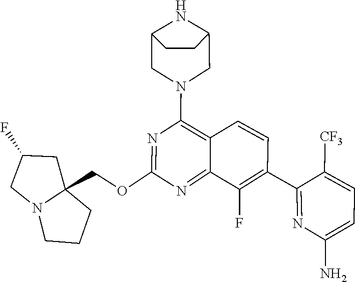 [SMILES: Nc1ccc(C(F)(F)F)c(-c2ccc3c(N4CC5CCC(C4)N5)nc(OC[C@@]45CCCN4C[C@H](F)C5)nc3c2F)n1] | 6-(4-((1R,5S)-3,8- diazabicyclo[3.2.1] octan-3-yl)-8- fluoro-2- (((2R,7aS)-2- fluorotetrahydro- 1H-pyrrolizin- 7a(5H)- yl)methoxy) quinazolin-7-yl)-5- (trifluoromethyl) pyridin-2-amine | 576.2 (M + H)⁺ | ¹H NMR (400 MHz, METHANOL-d₄) δ ppm 7.82- 7.99 (m, 2 H), 7.29-7.47 (m, 1 H), 6.74-6.84 (m, 1 H), 5.47- 5.70 (m, 1 H), 4.71 (d, J = 4.4 Hz, 4 H), 4.26 (br s, 2 H), 3.82-4.10 (m, 5 H), 3.43-3.58 (m, 1 H), 2.53-2.81 (m, 2 H), 2.31-2.52 (m, 3 H), 2.17 (s, 5 H). |
| 224 | 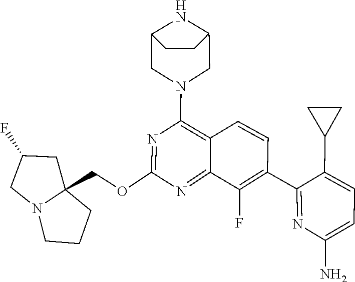 [SMILES: Nc1ccc(C2CC2)c(-c2ccc3c(N4CC5CCC(C4)N5)nc(OC[C@@]45CCCN4C[C@H](F)C5)nc3c2F)n1] | 6-(4-((1R,5S)-3,8- diazabicyclo[3.2.1] octan-3-yl)-8- fluoro-2- (((2R,7aS)-2- fluorotetrahydro- 1H-pyrrolizin- 7a(5H)- yl)methoxy) quinazolin-7-yl)-5- cyclopropylpyridin- 2-amine | 548.2 (M + H)⁺ | ¹H NMR (400 MHz, METHANOL-d₄) δ ppm 7.98- 8.06 (m, 1 H), 7.68-7.76 (m, 1 H), 7.49-7.60 (m, 1 H), 7.04- 7.12 (m, 1 H), 5.48-5.71 (m, 1 H), 4.72 (m, 4 H), 4.21-4.31 (m, 2 H), 3.79-4.15 (m, 6 H), 3.41- 3.62 (m, 1 H), 2.56-2.84 (m, 2 H), 2.32-2.53 (m, 4 H), 2.10- 2.30 (m, 6 H), 1.62-1.78 (m, 1 H), 0.83-0.93 (m, 2 H), 0.65- 0.77 (m, 2 H). |
| 225 | 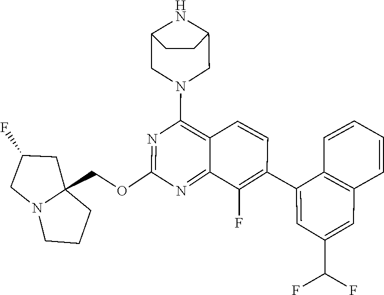 [SMILES: Fc1c(-c2cc(C(F)F)cc3ccccc23)ccc2c(N3CC4CCC(C3)N4)nc(OC[C@@]34CCCN3C[C@H](F)C4)nc12] | 4-((1R,5S)-3,8- diazabicyclo[3.2.1] octan-3-yl)-7-(3- (difluoromethyl) naphthalen-1-yl)-8- fluoro-2- (((2R,7aS)-2- fluorotetrahydro- 1H-pyrrolizin- 7a(5H)- yl)methoxy) quinazoline | 592.2 (M + H)⁺ | ¹H NMR (400 MHz, METHANOL-d₄) δ ppm 8.23- 8.28 (m, 1 H) 8.09-8.16 (m, 1 H) 7.97-8.06 (m, 1 H) 7.62-7.71 (m, 3 H) 7.56-7.63 (m, 1 H) 7.47- 7.54 (m, 1 H) 7.03 (s, 1 H) 5.47- 5.70 (m, 1 H) 4.62-4.78 (m, 4 H) 4.22-4.33 (m, 2 H) 3.81-4.13 (m, 5 H) 3.44-3.55 (m, 1 H) 2.54- 2.80 (m, 2 H) 2.28-2.52 (m, 3 H) 2.20 (br s, 5 H). |
| 226 | 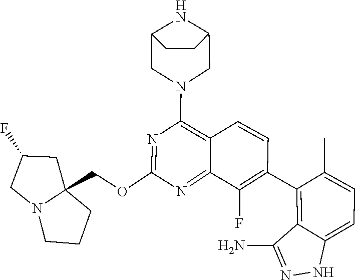 [SMILES: Cc1ccc2[nH]nc(N)c2c1-c1ccc2c(N3CC4CCC(C3)N4)nc(OC[C@@]34CCCN3C[C@H](F)C4)nc2c1F] | 4-(4-((1R,5S)-3,8- diazabicyclo[3.2.1] octan-3-yl)-8- fluoro-2- (((2R,7aS)-2- fluorotetrahydro- 1H-pyrrolizin- 7a(5H)- yl)methoxy) quinazolin-7-yl)-5- methyl-1H- indazol-3-amine | 561.2 (M + H)⁺ | ¹H NMR (400 MHz, METHANOL-d₄) δ ppm 8.00 (d, J = 8.6 Hz, 1 H), 7.30-7.48 (m, 3 H), 5.46-5.69 (m, 1 H), 4.71- 4.92 (m, 4 H), 4.28 (br s, 2 H), 3.79-4.11 (m, 5 H), 3.44-3.58 (m, 1 H), 2.56-2.81 (m, 2 H), 2.31-2.55 (m, 3 H), 2.20 (s, 8 H). |

### 3-(4-((1R,5S)-3,8-Diazabicyclo[3.2.1]octan-3-yl)-6,8-difluoro-2-(((2R,7aS)-2-fluorotetrahydro-1H-pyrrolizin-7a(5H)-yl)methoxy)quinazolin-7-yl)-2-cyclopropyl-5-hydroxybenzonitrile (Example 227

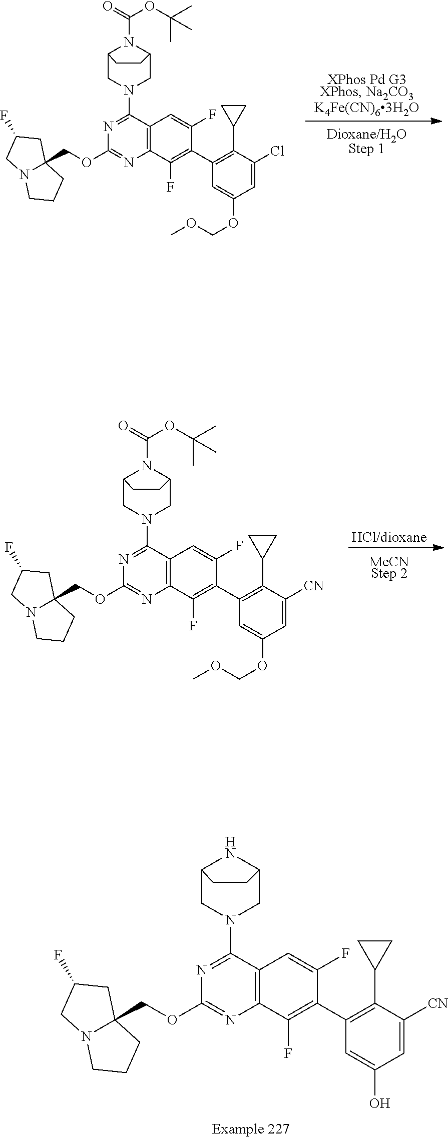

Step 1: tert-Butyl (1R,5S)-3-(7-(3-cyano-2-cyclopropyl-5-(methoxymethoxy)phenyl)-6,8-difluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate. A vial was charged with tert-butyl (1R,5S)-3-(7-(3-chloro-2-cyclopropyl-5-(methoxymethoxy)phenyl)-6,8-difluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate (41 mg, 0.055 mmol, synthesized in a similar way as Example 109), sodium carbonate (0.7 mg, 6.89 μmol), potassium ferrocyanide trihydrate (12 mg, 0.03 mmol), 2-dicyclohexylphosphino-2′,4′,6′,-triisopropylbiphenyl (5 mg, 0.011 mmol) and (2-dicyclohexylphosphino-2′,4′,6′-triisopropyl-1,1′-biphenyl)[2-(2′-amino-1,1′-biphenyl)]palladium(II) methanesulfonate (5 mg, 5.51 μmol) in dioxane (0.3 mL) and water (0.3 mL). The vial was degassed and purged with nitrogen. The reaction was then sealed and stirred at 85° C. Upon completion, the reaction was brought to room temperature and concentrated under reduced pressure. The crude mixture was then purified by column chromatography on silica gel, eluting with a gradient of 0-50% 3:1 EtOAc/EtOH in heptane with 2% triethylamine additive to provide tert-butyl (1R,5S)-3-(7-(3-cyano-2-cyclopropyl-5-(methoxymethoxy)phenyl)-6,8-difluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate as a white solid, which was used in the next step without further purification.

Step 2: 3-(4-((1R,5S)-3,8-Diazabicyclo[3.2.1]octan-3-yl)-6,8-difluoro-2-(((2R,7aS)-2-fluorotetrahydro-1H-pyrrolizin-7a(5H)-yl)methoxy)quinazolin-7-yl)-2-cyclopropyl-5-hydroxybenzonitrile. The above product tert-Butyl (1R,5S)-3-(7-(3-cyano-2-cyclopropyl-5-(methoxymethoxy)phenyl)-6,8-difluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate was dissolved in MeCN (2.5 mL). HCl solution (4 M in dioxane, 0.3 mL, 1.38 mmol) was added and the reaction was stirred at 23° C. After 1 h, the reaction was cooled to room temperature and concentrated under reduced pressure to provide a crude mixture. The crude mixture was then purified by reverse phase HPLC to provide 3-(4-((1R,5S)-3,8-diazabicyclo[3.2.1]octan-3-yl)-6,8-difluoro-2-(((2R,7aS)-2-fluorotetrahydro-1H-pyrrolizin-7a(5H)-yl)methoxy)quinazolin-7-yl)-2-cyclopropyl-5-hydroxybenzonitrile as a TFA salt and yellow liquid (2.2 mg, 0.004 mmol, 7% combined yield over 2 steps). m/z (ESI, +ve ion): 591.2 (M+H). ¹H NMR (400 MHz, METHANOL-d₄) δ ppm 7.55-7.65 (m, 1H), 7.14-7.19 (m, 1H), 6.96-7.03 (m, 1H), 5.20-5.41 (m, 1H), 4.36-4.52 (m, 2H), 4.19-4.32 (m, 2H), 3.52-3.70 (m, 4H), 3.15-3.29 (m, 3H), 2.96-3.08 (m, 1H), 2.09-2.44 (m, 3H), 1.75-2.07 (m, 8H), 0.61-0.74 (m, 2H), 0.31-0.43 (m, 2H).

### 6-(4-((1R,5S)-3,8-Diazabicyclo[3.2.1]octan-3-yl)-8-fluoro-2-(((2R,7aS)-2-fluorotetrahydro-1H-pyrrolizin-7a(5H)-yl)methoxy)quinazolin-7-yl)-4-chloro-5-(trifluoromethyl)pyridin-2-amine (Example 228

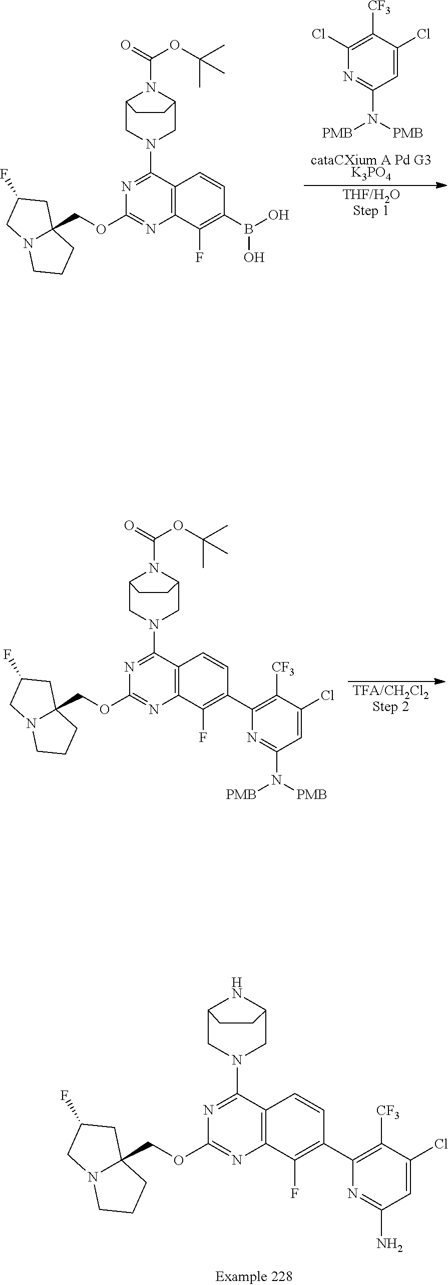

Step 1: tert-Butyl (1R,5S)-3-(7-(6-(bis(4-methoxybenzyl)amino)-4-chloro-3-(trifluoromethyl)pyridin-2-yl)-8-fluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate. (4-((1R,5S)-8-(tert-Butoxycarbonyl)-3,8-diazabicyclo[3.2.1]octan-3-yl)-8-fluoro-2-(((2R,7aS)-2-fluorotetrahydro-1H-pyrrolizin-7a(5H)-yl)methoxy)quinazolin-7-yl)boronic acid (0.12 g, 0.21 mmol), cataCXium A Pd G3 (8.0 mg, 10.6 μmol, Sigma Aldrich Corporation), 4,6-dichloro-N,N-bis(4-methoxybenzyl)-5-(trifluoromethyl)pyridin-2-amine (50 mg, 0.11 mmol, Intermediate R) and phosphoric acid tripotassium salt monohydrate (73 mg, 0.32 mmol) were stirred at 70° C. in degassed water (92 μL) and tetrahydrofuran (0.9 mL) for 6 h. Volatiles were removed in vacuo and the crude material was purified by column chromatography on silica gel, eluting with a gradient of 0-20% MeOH in DCM, with 0.5% 2 N NH₃ in MeOH to yield tert-butyl (1R,5S)-3-(7-(6-(bis(4-methoxybenzyl)amino)-4-chloro-3-(trifluoromethyl)pyridin-2-yl)-8-fluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate, which was used in the next step directly.

Step 2: 6-(4-((1R,5S)-3,8-Diazabicyclo[3.2.1]octan-3-yl)-8-fluoro-2-(((2R,7aS)-2-fluorotetrahydro-1H-pyrrolizin-7a(5H)-yl)methoxy)quinazolin-7-yl)-4-chloro-5-(trifluoromethyl)pyridin-2-amine. tert-Butyl (1R,5S)-3-(7-(6-(bis(4-methoxybenzyl)amino)-4-chloro-3-(trifluoromethyl)pyridin-2-yl)-8-fluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate was stirred in TFA (1.0 mL) at 50° C. for 4 h. Volatiles were removed in vacuo and the crude residue was purified by reverse phase HPLC to yield 6-(4-((1R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)quinazolin-7-yl)-4-chloro-5-(trifluoromethyl)pyridin-2-amine as tetrakis(2,2,2-trifluoroacetate) and colourless glass (18 mg, 0.017 mmol, 16% combined yield over 2 steps). m/z (ESI, +ve ion): 610.0 (M+H⁺). ¹H NMR (400 MHz, METHANOL-d₄) δ ppm 7.90 (d, J=8.4 Hz, 1H), 7.39 (dd, J=8.7, 6.8 Hz, 1H), 6.84 (s, 1H), 5.50-5.68 (m, 1H), 4.70-4.73 (m, 3H), 4.26 (br s, 2H), 3.82-4.09 (m, 5H), 3.46-3.56 (m, 1H), 2.11-2.81 (m, 12H).

### 6-(4-((1R,5S)-3,8-Diazabicyclo[3.2.1]octan-3-yl)-8-fluoro-2-(((2R,7aS)-2-fluorotetrahydro-1H-pyrrolizin-7a(5H)-yl)methoxy)quinazolin-7-yl)-4-methyl-5-(trifluoromethyl)pyridin-2-amine (Example 229

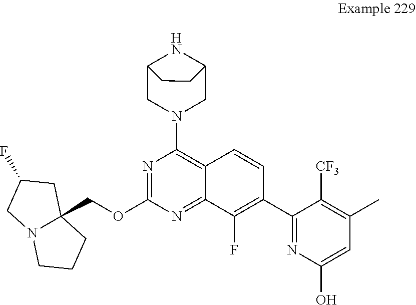 [SMILES: Cc1cc(O)nc(-c2ccc3c(N4CC5CCC(C4)N5)nc(OC[C@@]45CCCN4C[C@H](F)C5)nc3c2F)c1C(F)(F)F]

Synthesized in an analogous manner to Example 228 using Intermediate S. The product was isolated as the corresponding tetrakis(2,2,2-trifluoroacetate). m/z (ESI, +ve ion): 590.2 (M+H⁺). ¹H NMR (400 MHz, METHANOL-d₄) δ ppm 7.95 (d, J=8.7 Hz, 1H), 7.42 (dd, J=8.6, 6.7 Hz, 1H), 6.83 (s, 1H), 5.50-5.69 (m, 1H), 4.69-4.78 (m, 4H), 4.26 (br s, 2H), 4.00-4.10 (m, 1H), 3.87-3.98 (m, 4H), 3.46-3.55 (m, 1H), 2.34-2.80 (m, 9H), 2.14-2.27 (m, 5H).

### 4-(4-((1R,5S)-3,8-diazabicyclo[3.2.1]octan-3-yl-8-fluoro-2-(((2R,7aS)-2-fluorotetrahydro-1H-pyrrolizin-7a(5H)-yl)methoxy)-5-methylquinazolin-7-yl)-5-ethyl-6-fluoronaphthalen-2-ol (Example 230

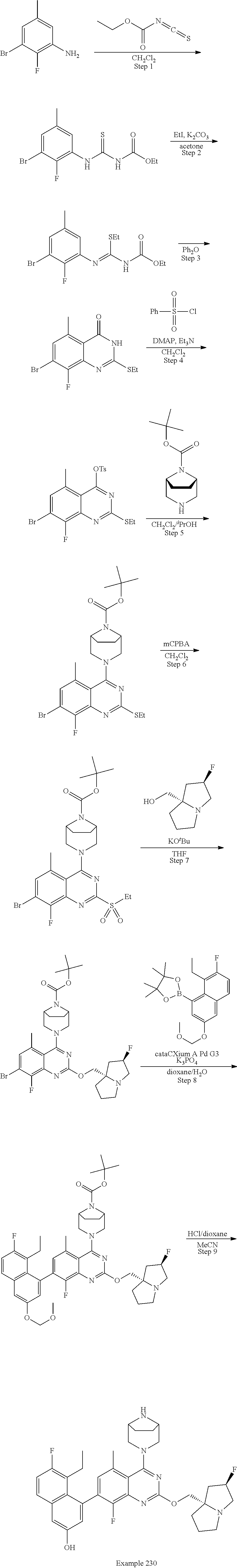

Step 1: To a stirring solution of 3-bromo-2-fluoro-5-methylaniline (1.00 g, 4.90 mmol, eNovation Chemicals LLC) in dichloromethane (8.0 mL) cooled with an ice-bath was added a solution of ethoxycarbonyl isothiocyanate (0.60 g, 4.90 mmol, Sigma-Aldrich Corporation) in dichloromethane (5.0 mL). The resulting mixture was stirred at 0° C. for 5S min and then at rt for 4 h. Volatiles were removed and the crude was taken onto the next step directly. m/z (ESI, +ve ion): 335.0 (M+H)⁺. ¹H NMR (CHLOROFORM-d, 400 MHz) δ 11.53 (br s, 1H), 7.9-8.3 (m, 2H), 7.25 (br d, 1H, J=1.5 Hz), 4.32 (q, 2H, J=7.1 Hz), 2.35 (s, 3H), 1.37 (t, 3H, J=7.1 Hz).

Step 2: To a stirring solution of the above product in acetone (30 mL) at rt was added potassium carbonate (1.00 g, 7.35 mmol, Sigma-Aldrich Corporation), followed by iodoethane (0.90 g, 0.5 mL, 5.88 mmol, Sigma-Aldrich Corporation). The resulting mixture was stirred at rt overnight. The solid was removed by filtration and the filtrate was concentrated under reduced pressure. The residue was purified by column chromatography on silica gel, eluting with a gradient of 0-40% EtOAc in heptane to give the desired product (1.60 g, 4.32 mmol, 88% combined yield over 2 steps) as a colorless liquid. m/z (ESI, +ve ion): 363.0 (M+H)⁺.

Step 3: A solution of the above product (1.70 g, 4.87 mmol) in diphenyl ether (8.0 mL) was subjected to microwave irradiation at 210° C. for 3 h. The crude mixture was purified by column chromatography on silica gel, eluting with a gradient of 0-60% EtOAc in heptane to give 7-bromo-2-(ethylthio)-8-fluoro-5-methylquinazolin-4(3H)-one (0.33 g, 1.03 mmol, 21% yield) as a white solid. m/z (ESI, +ve ion): 317.0 (M+H)⁺. ¹H NMR (CHLOROFORM-d, 400 MHz) δ 9.28 (br s, 1H), 7.29 (dd, 1H, J=0.7, 6.2 Hz), 3.33 (q, 2H, J=7.4 Hz), 2.77 (d, 3H, J=0.8 Hz), 1.47 (t, 3H, J=7.4 Hz).

Step 4: To a stirring solution of 7-bromo-2-(ethylthio)-8-fluoro-5-methylquinazolin-4(3H)-one (0.10 g, 0.32 mmol), triethylamine (80 mg, 0.1 mL, 0.79 mmol, Sigma-Aldrich Corporation), and 4-(dimethylamino)pyridine (3.9 mg, 0.03 mmol, Sigma-Aldrich Corporation) in DCM (2.5 mL) was added p-toluenesulfonyl chloride (0.12 mg, 0.63 mmol, Sigma-Aldrich Corporation) at rt. The resulting mixture was stirred at rt for 2 h. The crude mixture was purified by column chromatography on silica gel, eluting with a gradient of 0-50% EtOAc in heptane to give 7-bromo-2-(ethylthio)-8-fluoro-5-methylquinazolin-4-yl 4-methylbenzenesulfonate (99 mg, 0.21 mmol, 67% yield) as a colorless film. m/z (ESI, +ve ion): 470.8 (M+H)⁺.

Step 5: To a mixture of 7-bromo-2-(ethylthio)-8-fluoro-5-methylquinazolin-4-yl 4-methylbenzenesulfonate (0.10 g, 0.21 mmol) and tert-butyl (1R,5S)-3,8-diazabicyclo[3.2.1]octane-8-carboxylate (54 mg, 0.26 mmol, PharmaBlocks) was added at rt isopropanol (2.0 mL) and DCM (0.20 mL). The resulting mixture was stirred at rt for 2.5 h. Additional amine (43 mg) was added and stirring was continued overnight. The crude mixture was purified by column chromatography on silica gel, eluting with a gradient of 0-14% MeOH in DCM to give tert-butyl (1R,5S)-3-(7-bromo-2-(ethylthio)-8-fluoro-5-methylquinazolin-4-yl)-3,8-diazabicyclo[3.2.1]octane-8-carboxylate (0.11 g, 0.21 mmol, 97% yield) as a white solid. m/z (ESI, +ve ion): 511.0 (M+H)⁺.

Step 6: To a stirring solution of tert-butyl (1R,5S)-3-(7-bromo-2-(ethylthio)-8-fluoro-5-methylquinazolin-4-yl)-3,8-diazabicyclo[3.2.1]octane-8-carboxylate (0.11 g, 0.21 mmol) in DCM (3.0 mL) was added 3-chloroperoxybenzoic acid (48 mg, 0.22 mmol, Sigma-Aldrich Corporation) in one portion at 0° C. The resulting mixture was stirred at 0° C. for 1.5 h. The crude mixture was purified by column chromatography on silica gel, eluting with a gradient of 0-100% EtOAc in heptane to give tert-butyl (1R,5S)-3-(7-bromo-2-(ethylsulfonyl)-8-fluoro-5-methylquinazolin-4-yl)-3,8-diazabicyclo[3.2.1]octane-8-carboxylate (62 mg, 0.11 mmol, 56% yield) as a colorless film. m/z (ESI, +ve ion): 543.0 (M+H)⁺.

Step 7: To a stirring solution of ((2R,7aS)-2-fluorotetrahydro-1H-pyrrolizin-7a(5H)-yl)methanol (33 mg, 0.21 mmol, LabNetwork) in THF (1.5 mL) was added dropwise potassium tert-butoxide in THF (1.0 M, 0.2 mL, 0.23 mmol, Sigma-Aldrich Corporation) under nitrogen at 0° C. The resulting mixture was stirred at 0° C. for 15 min and was added slowly to a stirring solution of tert-butyl (1R,5S)-3-(7-bromo-2-(ethylsulfonyl)-8-fluoro-5-methylquinazolin-4-yl)-3,8-diazabicyclo[3.2.1]octane-8-carboxylate (62 mg, 0.11 mmol) in tetrahydrofuran (2.0 mL) under nitrogen at 0° C. The resulting mixture was stirred at 0° C. for 30 min. The crude mixture was purified by column chromatography on silica gel, eluting with a gradient of 0-18% MeOH in DCM to give tert-butyl (1R,5S)-3-(7-bromo-8-fluoro-2-(((2R,7aS)-2-fluorotetrahydro-1H-pyrrolizin-7a(5H)-yl)methoxy)-5-methylquinazolin-4-yl)-3,8-diazabicyclo[3.2.1]octane-8-carboxylate (45 mg, 0.07 mmol, 65% yield) as a colorless film. m/z (ESI, +ve ion): 719.2 (M+H)⁺.

Step 8: In a 5-mL cone-shaped microwave reaction vessel was placed tert-butyl (1R,5S)-3-(7-bromo-8-fluoro-2-(((2R,7aS)-2-fluorotetrahydro-1H-pyrrolizin-7a(5H)-yl)methoxy)-5-methylquinazolin-4-yl)-3,8-diazabicyclo[3.2.1]octane-8-carboxylate (40 mg, 0.07 mmol), 2-(8-ethyl-7-fluoro-3-(methoxymethoxy)naphthalen-1-yl)-4,4,5,5-tetramethyl-1,3,2-dioxaborolane (47 mg, 0.13 mmol, LabNetwork), cataCXium A Pd G3 (9.6 mg, 0.01 mmol, Sigma Aldrich Corporation), and potassium phosphate tribasic (35 mg, 0.16 mmol, Acros Organics) followed by 1,4-dioxane (4.0 mL) and water (0.8 mL). The resulting mixture was purged with nitrogen for 10 min before being sealed and irradiated under microwave at 80° C. for 2 h. Volatiles were removed under reduced pressure. The crude residue was purified by column chromatography on silica gel, eluting with a gradient of 0-17% MeOH in DCM to give tert-butyl (1R,5S)-3-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)-5-methylquinazolin-4-yl)-3,8-diazabicyclo[3.2.1]octane-8-carboxylate as an off-white solid, which was directly taken onto the next step. m/z (ESI, +ve ion): 762.4 (M+H)⁺.

Step 9: To a stirring solution of tert-butyl (1R,5S)-3-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)-5-methylquinazolin-4-yl)-3,8-diazabicyclo[3.2.1]octane-8-carboxylate (55 mg, 0.07 mmol) in MeCN (2.0 mL) was added at rt HCl in dioxane (4.0 M, 0.6 mL, 2.53 mmol, Sigma-Aldrich Corporation). The resulting mixture was stirred at rt for 1 h. Volatiles were removed under reduced pressure. The crude residue was dissolved in MeOH/DCM and cooled in an ice bath before ammonium hydroxide (1.0 mL) was added to neutralize. The resulting mixture was purified by column chromatography on silica gel, eluting with a gradient of 2-20% MeOH with 0.5% ammonium hydroxide in DCM to give 4-(4-((1R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)-5-methylquinazolin-7-yl)-5-ethyl-6-fluoronaphthalen-2-ol (28 mg, 0.05 mmol, 63% yield) as a white solid. m/z (ESI, +ve ion): 618.40 (M+H)⁺. ¹H NMR (METHANOL-d₄, 400 MHz) δ 7.63 (dd, 1H, J=6.0, 9.1 Hz), 7.1-7.3 (m, 2H), 7.04 (br d, 1H, J=6.3 Hz), 6.97 (d, 1H, J=2.5 Hz), 5.2-5.4 (m, 1H), 4.3-4.7 (m, 1H), 4.2-4.3 (m, 1H), 4.2-4.2 (m, 1H), 3.9-4.2 (m, 1H), 3.74 (br d, 1H, J=2.1 Hz), 3.4-3.6 (m, 3H), 3.1-3.3 (m, 3H), 3.00 (dt, 1H, J=6.0, 9.2 Hz), 2.3-2.7 (m, 5H), 1.8-2.3 (m, 6H), 1.2-1.8 (m, 4H), 0.77 (dt, 3H, J=1.5, 7.3 Hz).

### 4-(4-((1R,5S)-3,8-diazabicyclo[3.2.1]octan-3-yl)-5,8-difluoro-2-(((2R,7aS)-2-fluorotetrahydro-1H-pyrrolizin-7a(5H)-yl)methoxy)quinazolin-7-yl)-5-ethyl-6-fluoronaphthalen-2-ol (Example 231

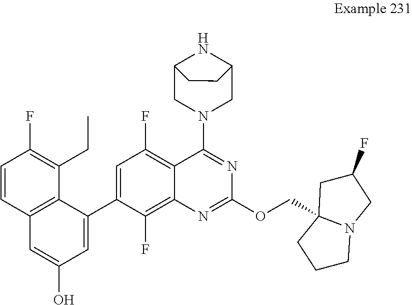 [SMILES: CCc1c(F)ccc2cc(O)cc(-c3cc(F)c4c(N5CC6CCC(C5)N6)nc(OC[C@@]56CCCN5C[C@H](F)C6)nc4c3F)c12]

Synthesized in an analogous manner to Example 230 from 3-bromo-2,5-difluoroaniline (eNovation Chemicals LLC). m/z (ESI, +ve ion): 622.3 (M+H)⁺. ¹H NMR (METHANOL-d₄, 400 MHz) δ 7.64 (dd, 1H, J=5.9, 9.0 Hz), 7.2-7.3 (m, 2H), 6.9-7.0 (m, 2H), 5.1-5.4 (m, 1H), 4.1-4.3 (m, 4H), 3.4-3.6 (m, 4H), 3.1-3.3 (m, 3H), 3.00 (dt, 1H, J=5.7, 9.4 Hz), 2.4-2.6 (m, 2H), 2.1-2.4 (m, 3H), 1.7-2.0 (m, 7H), 0.82 (dt, 3H, J=1.7, 7.3 Hz).

### (M)-4-(4-((1R,5S)-3,8-diazabicyclo[3.2.1]octan-3-yl)-6,8-difluoro-2-(((2R,7aS)-2-fluorotetrahydro-1H-pyrrolizin-7a(5H)-yl)methoxy)quinazolin-7-yl)-5-ethyl-6-fluoronaphthalen-2-ol (Example 232

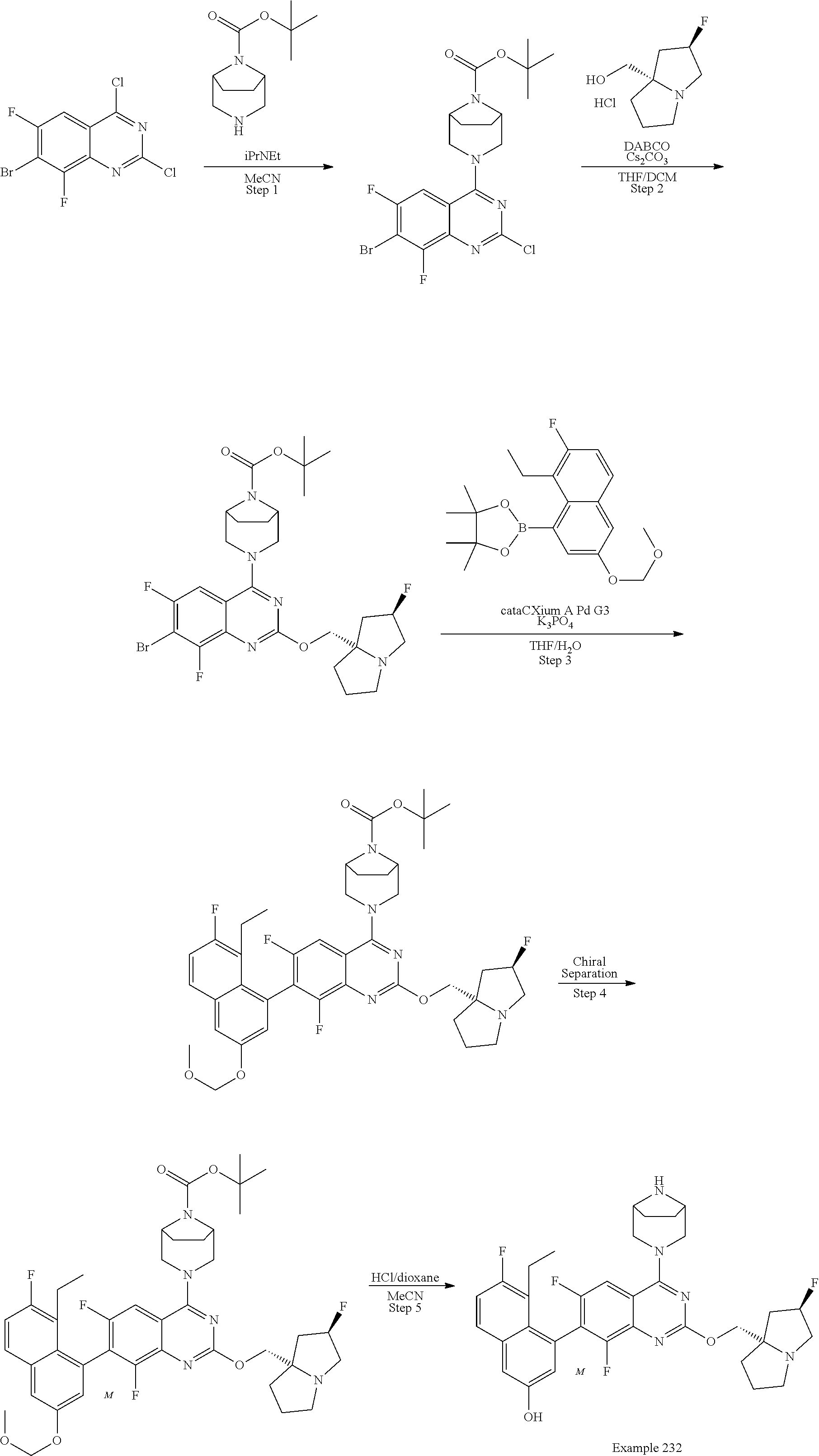

Step 1: tert-Butyl (1R,5S)-3-(7-bromo-2-chloro-6,8-difluoroquinazolin-4-yl)-3,8-diazabicyclo[3.2.1]octane-8-carboxylate. To a stirred solution of 7-bromo-2,4-dichloro-6,8-difluoroquinazoline (1.00 g, 3.19 mmol, Enamine) in acetonitrile (16 mL) was added 8-Boc-3,8-diaza-bicyclo[3.2.1]octane (0.70 g, 3.34 mmol, Chem-Impex International, Inc.) and DIPEA (1.2 g, 1.7 mL, 9.56 mmol, Sigma-Aldrich Corporation). The reaction was then stirred for 1.5 h. The reaction was then diluted with water (50 mL) and brine (50 mL) and the aqueous layer was then extracted with CH₂Cl₂ (3×100 mL). The combined organic layers were dried with sodium sulfate, filtered and concentrated under reduced pressure to provide tert-butyl (1R,5S)-3-(7-bromo-2-chloro-6,8-difluoroquinazolin-4-yl)-3,8-diazabicyclo[3.2.1]octane-8-carboxylate (1.70 g) as a light yellow solid. The product was used without further purification. m/z (ESI, +ve ion): 488.8 (M+H)⁺.

Step 2: tert-Butyl (1R,5S)-3-(7-bromo-6,8-difluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate. To a scintillation vial was added tert-butyl (1R,5S)-3-(7-bromo-2-chloro-6,8-difluoroquinazolin-4-yl)-3,8-diazabicyclo[3.2.1]octane-8-carboxylate (1.90 g, 3.82 mmol), cesium carbonate (3.70 g, 11.5 mmol, Sigma-Aldrich Corporation) and 1,4-diazabicyclo[2.2.2]octane (8.6 mg, 0.76 mmol, Sigma-Aldrich Corporation). The solids were then suspended in N,N-dimethylformamide (10 mL) and tetrahydrofuran (20 mL), and ((2R,7aS)-2-fluorotetrahydro-1H-pyrrolizin-7a(5H)-yl)methanol (0.90 g, 5.35 mmol, PharmaBlock) was added. The reaction mixture was then stirred at 35° C. After stirring for 22 h the reaction was diluted with water (50 mL) and transferred to a separatory funnel. The layers were separated and the aqueous layer was extracted with EtOAc (3×50 mL). The organic layers were combined, dried with sodium sulfate, filtered, and concentrated under reduced pressure. The crude reaction mixture was then purified by column chromatography on silica gel, eluting with a gradient of 0-50% 3:1 EtOAc/EtOH with 2% triethylamine in heptane to provide tert-butyl (1R,5S)-3-(7-bromo-6,8-difluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate (1.90 g, 3.16 mmol, 83% yield) as a yellow solid. m/z (ESI, +ve ion): 611.8 (M+H)⁺.

Step 3: tert-Butyl (1R,5S)-3-(7-(8-ethyl-7-fluoro-3-(methoxymethoxy)naphthalen-1-yl)-6,8-difluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate. A vial was charged with tert-butyl (1R,5S)-3-(7-bromo-6,8-difluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate (1.40 g, 2.20 mmol), 2-(8-ethyl-7-fluoro-3-(methoxymethoxy)naphthalen-1-yl)-4,4,5,5-tetramethyl-1,3,2-dioxaborolane (1.60 g, 4.41 mmol, LabNetwork), potassium phosphate (1.40 g, 6.61 mmol), and cataCXium A Pd G3 (0.3 g, 0.44 mmol, Sigma Aldrich Corporation). The vial was purged with nitrogen and the reactants were suspended in degassed tetrahydrofuran (20 mL) and water (2.0 mL). The reaction was then sealed and heated to 70° C. After stirring overnight, the reaction was cooled to room temperature and concentrated under reduced pressure to afford a crude black oil. The oil was then purified by column chromatography on silica gel, eluting with a gradient of 0-50% 3:1 EtOAc/EtOH (with 2% triethylamine) in heptane to provide tert-butyl (1R,5S)-3-(7-(8-ethyl-7-fluoro-3-(methoxymethoxy)naphthalen-1-yl)-6,8-difluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate (1.50 g, 1.90 mmol, 86% yield) as an off-white foam. m/z (ESI, +ve ion): 766.2 (M+H)⁺.

Step 4: (M)-tert-Butyl (1R,5S)-3-(7-(8-ethyl-7-fluoro-3-(methoxymethoxy)naphthalen-1-yl)-6,8-difluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate. tert-Butyl (1R,5S)-3-(7-(8-ethyl-7-fluoro-3-(methoxymethoxy)naphthalen-1-yl)-6,8-difluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate (1.00 g) was purified by SFC using a ChromegaChiral CCC column, 21×150 mm, mobile phase of 80% CO₂ and 20% isopropanol w/0.2% diethylamine using a flow rate of 80 ml/min to generate 362 mg of peak 1 with ee of >99% and 386 mg of peak 2 with ee of >96%. Peak assignment determined by SFC using ChiralChromega CCC column, mobile phase 80% CO2 and 20% methanol w/0.2% diethylamine. Peak 2 was assigned (M)-tert-butyl (1R,5S)-3-(7-(8-ethyl-7-fluoro-3-(methoxymethoxy)naphthalen-1-yl)-6,8-difluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate.

Step 5: (M)-4-(4-((1R,5S)-3,8-diazabicyclo[3.2.1]octan-3-yl)-6,8-difluoro-2-(((2R,7aS)-2-fluorotetrahydro-1H-pyrrolizin-7a(5H)-yl)methoxy)quinazolin-7-yl)-5-ethyl-6-fluoronaphthalen-2-ol. A vial was charged with (M)-tert-butyl (1R,5S)-3-(7-(8-ethyl-7-fluoro-3-(methoxymethoxy)naphthalen-1-yl)-6,8-difluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate (42 mg, 0.05 mmol) and the solid was dissolved in acetonitrile (1.7 mL). The solution was cooled to 0° C. and HCl in dioxane (4 M, 0.3 mL, 1.36 mmol) was added. The reaction was stirred for 15 min, and then warmed to rt. After stirring for an additional 15 min, the reaction was concentrated under reduced pressure and purified via reverse phase HPLC to provide (A4)-4-(4-((1R,5S)-3,8-diazabicyclo[3.2.1]octan-3-yl)-6,8-difluoro-2-(((2R,7aS)-2-fluorotetrahydro-1H-pyrrolizin-7a(5H)-yl)methoxy)quinazolin-7-yl)-5-ethyl-6-fluoronaphthalen-2-ol as bis(2,2,2-trifluoroacetate) (31 mg, 0.04 mmol, 68% yield) as a yellow solid. m/z (ESI, +ve ion): 622.2 (M+H)⁺. ¹H NMR (400 MHz, METHANOL-d₄) δ ppm 7.71 (s, 2H) 7.34 (d, J=2.51 Hz, 1H) 7.28 (s, 1H) 6.94-7.03 (m, 1H) 5.44-5.71 (m, 1H) 4.70 (d, J=4.81 Hz, 4H) 4.24-4.34 (m, 2H) 3.82-4.11 (m, 5H) 3.43-3.57 (m, 1H) 2.53-2.82 (m, 3H) 2.30-2.51 (m, 4H) 2.20 (br s, 5H) 0.82 (t, J=7.42 Hz, 3H).

### (M)-4-(4-((1S,4S)-2,5-Diazabicyclo[2.2.2]octan-2-yl)-6,8-difluoro-2-(((2R,7aS)-2-fluorotetrahydro-1H-pyrrolizin-7a(5H)-yl)methoxy)quinazolin-7-yl)-5,6-difluoronaphthalen-2-ol (Example 233

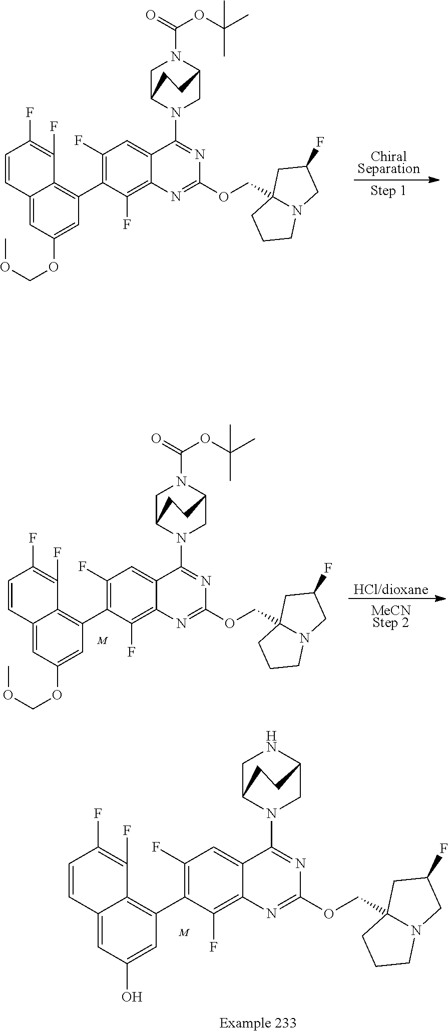

Step 1: (M)-tert-Butyl (1S,4S)-5-(7-(7,8-difluoro-3-(methoxymethoxy)naphthalen-1-yl)-6,8-difluoro-2-(((2R,7aS)-2-fluorotetrahydro-1H-pyrrolizin-7a(5H)-yl)methoxy)quinazolin-4-yl)-2,5-diazabicyclo[2.2.2]octane-2-carboxylate. tert-Butyl (1S,4S)-5-(7-(7,8-difluoro-3-(methoxymethoxy)naphthalen-1-yl)-6,8-difluoro-2-(((2R,7aS)-2-fluorotetrahydro-1H-pyrrolizin-7a(5H)-yl)methoxy)quinazolin-4-yl)-2,5-diazabicyclo[2.2.2]octane-2-carboxylate (60 mg, synthesized in an analogous manner to Example 233) was purified by SFC using a (S,S) Whelk-O1 21×500, 5 micron, mobile phase of 35% methanol with 0.2% triethylamine using a flowrate of 80 mL/min to generate 22 mg of peak 1 with ee of >99% and 18 mg of peak 2 with ee of >99%. Peak assignment determined by SFC with (S,S) Whelk-O1 and 40% methanol with 0.2% triethylamine. Peak 2 was assigned (M)-tert-butyl (1S,4S)-5-(7-(7,8-difluoro-3-(methoxymethoxy)naphthalen-1-yl)-6,8-difluoro-2-(((2R,7aS)-2-fluorotetrahydro-1H-pyrrolizin-7a(5H)-yl)methoxy)quinazolin-4-yl)-2,5-diazabicyclo[2.2.2]octane-2-carboxylate.

Step 2: (M)-4-(4-((1S,4S)-2,5-Diazabicyclo[2.2.2]octan-2-yl)-6,8-difluoro-2-(((2R,7aS)-2-fluorotetrahydro-1H-pyrrolizin-7a(5H)-yl)methoxy)quinazolin-7-yl)-5,6-difluoronaphthalen-2-ol. A vial was charged with (M)-tert-butyl (1S,4S)-5-(7-(7,8-difluoro-3-(methoxymethoxy)naphthalen-1-yl)-6,8-difluoro-2-(((2R,7aS)-2-fluorotetrahydro-1H-pyrrolizin-7a(5H)-yl)methoxy)quinazolin-4-yl)-2,5-diazabicyclo[2.2.2]octane-2-carboxylate (18 mg, 0.02 mmol) and the solid was dissolved in acetonitrile (0.8 mL). The solution was cooled to 0° C. and HCl in dioxane (4 M, 0.2 mL, 0.60 mmol) was added. The reaction was stirred for 15 min, and then warmed to room temperature. After 40 min, the reaction was concentrated under reduced pressure. The crude yellow solid was then purified via reverse phase HPLC to provide (M)-4-(4-((1S,4S)-2,5-diazabicyclo[2.2.2]octan-2-yl)-6,8-difluoro-2-(((2R,7aS)-2-fluorotetrahydro-1H-pyrrolizin-7a(5H)-yl)methoxy)quinazolin-7-yl)-5,6-difluoronaphthalen-2-ol as tris(2,2,2-trifluoroacetate) salt (5.6 mg, 5.87 μmol, 24% yield) as a yellow solid. m/z (ESI, +ve ion): 611.8 (M+H)⁺. ¹H NMR (400 MHz, METHANOL-d₄) δ ppm 7.81-7.87 (m, 1H) 7.60-7.69 (m, 1H) 7.38-7.47 (m, 1H) 7.34-7.38 (m, 1H) 7.13-7.22 (m, 1H) 5.45-5.71 (m, 1H) 5.06-5.24 (m, 1H) 4.63-4.75 (m, 2H) 4.33-4.59 (m, 2H) 3.78-4.12 (m, 5H) 3.57-3.68 (m, 1H)3.43-3.54 (m, 1H)2.56-2.79 (m, 2H)2.30-2.56 (m, 4H) 2.03-2.30 (m, 4H).

### (M)-4-((1R,5S)-3,8-Diazabicyclo[3.2.1]octan-3-yl)-7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)quinazolin-6-ol. (Example 234

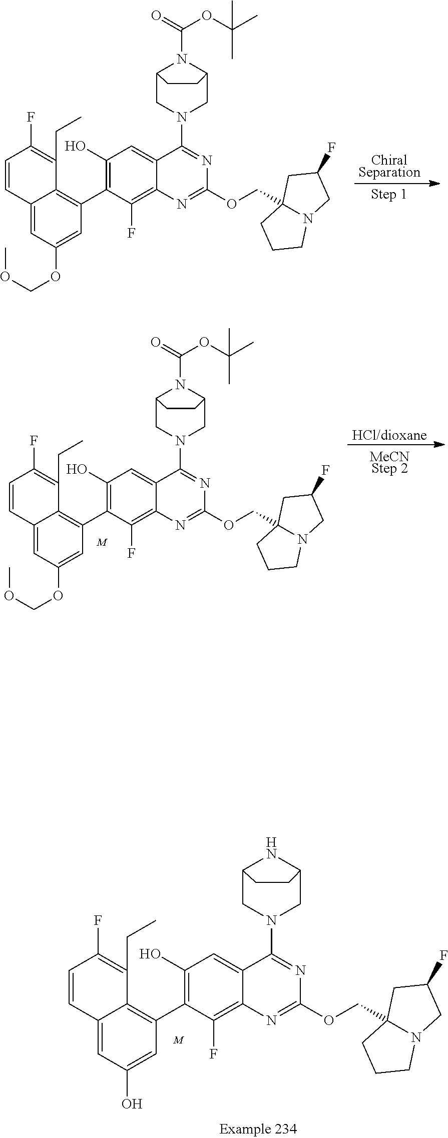

Step 1: (M)-tert-Butyl (1R,5S)-3-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)-6-hydroxyquinazolin-4-yl)-3,8-diazabicyclo[3.2.1]octane-8-carboxylate. tert-Butyl (1R,5S)-3-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)-6-hydroxyquinazolin-4-yl)-3,8-diazabicyclo[3.2.1]octane-8-carboxylate (0.12 g, synthesized in an analogous manner to Example 199) was purified by SFC using a Chiralpak AD, 21×250 mm 5 μm, column with a mobile phase of 30% 2-propanol with 0.2% triethylamine using a flowrate of 80 mL/min to generate 48.2 mg of peak 1 with an ee of >99% and 45.5 mg of peak 2 with an ee of >99%. Peak assignment determined by SFC with Chiralpak AD column with 30% 2-propanol with 0.2% triethylamine. Peak 1 was assigned (M)-tert-Butyl (1R,5S)-3-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)-6-hydroxyquinazolin-4-yl)-3,8-diazabicyclo[3.2.1]octane-8-carboxylate.

Step 2: (M)-4-((1R,5S)-3,8-Diazabicyclo[3.2.1]octan-3-yl)-7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)quinazolin-6-ol. A vial was charged with (M)-tert-Butyl (1R,5S)-3-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)-6-hydroxyquinazolin-4-yl)-3,8-diazabicyclo[3.2.1]octane-8-carboxylate (48 mg, 0.06 mmol), HCl in dioxane (4 M, 0.3 mL, 1.26 mmol) and MeOH (0.3 mL). The reaction was stirred at rt for 1 h. The reaction was concentrated under reduced pressure. The crude product was then purified via reverse phase HPLC to provide (M)-4-((1R,5S)-3,8-Diazabicyclo[3.2.1]octan-3-yl)-7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)quinazolin-6-ol as bis(2,2,2-trifluoroacetate) salt (36 mg, 0.04 mmol, 67% yield) as light-yellow solid. m/z (ESI, +ve ion): 620.0 (M+H)⁺. ¹H NMR (400 MHz, METHANOL-d₄) δ ppm 7.60-7.72 (m, 1H), 7.27-7.33 (m, 2H), 7.20-7.27 (m, 1H), 6.92 (d, J=2.5 Hz, 1H), 5.46-5.66 (m, 1H), 4.52-4.73 (m, 4H), 4.23-4.35 (m, 2H), 3.71-4.07 (m, 5H), 3.42-3.54 (m, 1H), 2.10-2.79 (m, 13H), 0.77-0.87 (m, 3H).

### Biological Evolution

Provided in this section is the biological evaluation of the specific examples provided herein.

KRAS G12D TR-FRET Assay

Compounds of interest were prepared in a dose-response titration in DMSO, and 80 nL were added via Labcyte Echo to each well of a 384-well plate (Perkin Elmer 6008280). The His-tagged KRAS G12D protein (Amgen) was diluted to 20 nM in Assay Buffer (20 mM HEPES, pH 7.4, 10 mM MgCl₂, 50 mM NaCl, 0.1% BSA, 0.01% Tween-20, 10 μM GDP) and 2 uL was added to the appropriate wells of the 384-well plate. The plate was incubated for 30 minutes at room temperature. Biotinylated KRPep-2d substrate (Amgen) was diluted to 20 nM in Assay Buffer and 2 μL was added to all wells and incubated for 1 h at room temperature. Detection Reagent (0.4 nM LANCE Eu-W1024 Anti-6xHis (Perkin Elmer AD0401), 5 nM streptavidin-d2 (Cisbio 610SADLA)) was prepared in Assay Buffer, then 4 μL was added to the plate and incubated for 1 h at room temperature. Plates were read using PerkinElmer EnVision (ex: 320 nm, em1: 665 nm, em2: 615 nm) and em1/em2 data was used to generate curve fits using a 4-parameter logistic model to calculate IC50 values.

### KRAS G12D Coupled Nucleotide Exchange Assay

Purified GDP-bound KRAS protein (aa 1-169), containing both G12D and C118A amino acid substitutions and an N-terminal His-tag, was pre-incubated in assay buffer (25 mM HEPES pH 7.4, 10 mM MgCl₂, and 0.01% Triton X-100) with a compound dose-response titration for 2 h. Following compound pre-incubation, purified SOS protein (aa 564-1049) and GTP (Roche 10106399001) were added to the assay wells and incubated for an additional 30 min. To determine the extent of inhibition of SOS-mediated nucleotide exchange, purified GST-tagged cRAF (aa 1-149), nickel chelate AlphaLISA acceptor beads (PerkinElmer AL108R), and AlphaScreen glutathione donor beads (PerkinElmer 6765302) were added to the assay wells and incubated for 10 minutes. The assay plates were then read on a PerkinElmer EnVision Multilabel Reader, using AlphaScreen® technology, and data were analyzed using a 4-parameter logistic model to calculate IC₅₀ values.

### Phospho-ERK1/2 MSD Assay

A-427 (ATCC® HTB-53™) cells were cultured in RPMI 1640 Medium (ThermoFisher Scientific 11875093) containing 10% fetal bovine serum (ThermoFisher Scientific 16000044) and 1× penicillin-streptomycin-glutamine (ThermoFisher Scientific 10378016). Sixteen hours prior to compound treatment, A-427 cells were seeded in 96-well cell culture plates at a density of 25,000 cells/well and incubated at 37° C., 5% CO₂. A compound dose-response titration was diluted in growth media, added to appropriate wells of a cell culture plate, and then incubated at 37° C., 5% CO₂ for 2 h. Following compound treatment, cells were washed with ice-cold Dulbecco's phosphate-buffered saline, no Ca²⁺ or Mg²⁺ (ThermoFisher Scientific 14190144), and then lysed in RIPA buffer (50 mM Tris-HCl pH 7.5, 1% Igepal, 0.5% sodium deoxycholate, 150 mM NaCl, and 0.5% sodium dodecyl sulfate) containing protease inhibitors (Roche 4693132001) and phosphatase inhibitors (Roche 4906837001). Phosphorylation of ERK1/2 in compound-treated lysates was assayed using Phospho-ERK1/2 Whole Cell Lysate kits (Meso Scale Discovery K151DWD) according to the manufacturer's protocol. Assay plates were read on a Meso Scale Discovery Sector Imager 6000, and data were analyzed using a 4-parameter logistic model to calculate IC₅₀ values.

### Phospho-ERK1/2 MSD Assay

AsPC-1 (ATCC® CRL-1682™) cells were cultured in RPMI 1640 Medium (ThermoFisher Scientific 11875093) containing 10% fetal bovine serum (ThermoFisher Scientific 16000044) and 1× penicillin-streptomycin-glutamine (ThermoFisher Scientific 10378016). Sixteen hours prior to compound treatment, AsPC-1 cells were seeded in 96-well cell culture plates at a density of 25,000 cells/well and incubated at 37° C., 5% CO₂. A compound dose-response titration was diluted in growth media, added to appropriate wells of a cell culture plate, and then incubated at 37° C., 5% CO₂ for 2 hours. Following compound treatment, cells were washed with ice-cold Dulbecco's phosphate-buffered saline, no Ca²⁺ or Mg²⁺ (ThermoFisher Scientific 14190144), and then lysed in RIPA buffer (50 mM Tris-HCl pH 7.5, 1% Igepal, 0.5% sodium deoxycholate, 150 mM NaCl, and 0.5% sodium dodecyl sulfate) containing protease inhibitors (Roche 4693132001) and phosphatase inhibitors (Roche 4906837001). Phosphorylation of ERK1/2 in compound-treated lysates was assayed using Phospho-ERK1/2 Whole Cell Lysate kits (Meso Scale Discovery K151DWD) according to the manufacturer's protocol. Assay plates were read on a Meso Scale Discovery Sector Imager 6000, and data were analyzed using a 4-parameter logistic model to calculate IC₅₀ values.

TABLE 10
Biochemical and cellular activity of examples.

| | | Avg 2 h | p-ERK | p-ERK |
|---|---|---|---|---|
| | TR-FRET | Coupled | (A427 | (AsPC- |
| | KRAS | Exchange | cells) | 1 cells) |
| | G12D IC₅₀ | IC₅₀ | IC₅₀ | IC₅₀ |
| Ex. # | (μM) | (μM) | (μM) | (μM) |
| 1 | 0.004 | 0.003 | 0.171 | 0.42 |
| 2 | 0.007 | 0.004 | 0.222 | 0.721 |
| 3 | 0.01 | 0.005 | 0.65 | 0.812 |
| 4 | 0.005 | 0.007 | 0.294 | 1.23 |
| 5 | 0.043 | 0.019 | 2.15 | 2.97 |
| 6 | 0.049 | 0.022 | 2.31 | 5.13 |
| 7 | 0.075 | 0.035 | 3.51 | 3.3 |
| 8 | 0.092 | 0.087 | 7.88 | 6.94 |
| 9 | 0.456 | 0.125 | >25 | >25 |
| 10 | 0.106 | 0.132 | 5.24 | 6.27 |
| 11 | 0.021 | 0.009 | 2.51 | 1.53 |
| 12 | 0.005 | 0.005 | 0.09 | 0.293 |
| 13 | 0.007 | 0.006 | 0.238 | 1.73 |
| 14 | 0.009 | 0.008 | 0.274 | 0.762 |
| 15 | 0.020 | 0.014 | 1.55 | 3.22 |
| 16 | 0.020 | 0.007 | NT | 4.82 |
| 17 | 0.026 | 0.017 | 1.29 | 0.949 |
| 18 | 0.06 | 0.018 | 1.59 | 2.96 |
| 19 | 0.058 | 0.028 | 1.79 | 2.09 |
| 20 | 0.087 | 0.04 | 7.28 | 5.56 |
| 21 | 0.314 | 0.078 | 18.1 | 9.08 |
| 22 | 0.017 | 0.014 | 2.66 | 2.35 |
| 23 | 0.044 | 0.019 | 10.3 | 2.6 |
| 24 | 0.031 | 0.016 | 3.91 | 1.39 |
| 25 | 0.058 | 0.017 | 5.05 | 14.8 |
| 26 | 0.034 | 0.018 | 1.11 | 0.692 |
| 27 | 0.047 | 0.021 | 0.865 | 5.23 |
| 28 | 0.09 | 0.044 | >10 | 3.61 |
| 29 | 0.161 | 0.08 | >10 | 10 |
| 30 | 0.559 | 0.158 | 17.7 | 21.5 |
| 31 | 0.546 | 0.259 | NT | NT |
| 32 | 0.005 | 0.002 | 0.341 | 0.712 |
| 33 | 0.008 | 0.003 | 0.291 | 0.509 |
| 34 | 0.047 | 0.022 | 2.57 | 2.98 |
| 35 | 4.7 | 1.82 | NT | NT |
| 36 | 0.483 | 0.082 | >10 | >10 |
| 37 | 0.193 | 0.073 | >10 | 6.7 |
| 38 | 0.195 | 0.042 | >10 | 6.31 |
| 39 | 0.02 | 0.009 | 0.569 | 1.89 |
| 40 | 0.011 | 0.006 | 0.686 | 2.39 |
| 41 | 0.002 | 0.002 | 0.074 | 0.182 |
| 42 | 0.003 | 0.003 | 0.077 | 0.223 |
| 43 | 0.007 | 0.002 | 0.016 | 0.09 |
| 44 | 0.002 | 0.002 | 0.019 | 0.114 |
| 45 | 0.004 | 0.002 | 0.025 | 0.19 |
| 46 | 0.002 | 0.002 | 0.006 | 0.039 |
| 47 | 0.004 | 0.003 | 0.011 | 0.031 |
| 48 | 0.004 | 0.004 | 0.043 | 0.157 |
| 49 | 0.018 | 0.009 | 0.652 | 3.6 |
| 50 | 0.011 | 0.007 | 1.01 | 4.47 |
| 51 | 0.003 | 0.001 | 0.136 | 0.185 |
| 52 | 0.003 | 0.003 | 0.041 | 0.107 |
| 53 | 0.016 | 0.003 | 0.608 | 0.919 |
| 54 | 0.004 | 0.003 | 0.795 | 1.22 |
| 55 | 0.008 | 0.007 | 1.15 | 1.66 |
| 56 | 0.005 | 0.004 | 0.023 | 0.109 |
| 57 | 0.005 | 0.003 | 0.222 | 0.213 |
| 58 | 0.006 | 0.005 | 4.19 | 2.46 |
| 59 | 0.008 | 0.011 | 3.38 | 4.16 |
| 60 | 0.008 | 0.004 | 0.53 | 0.418 |
| 61 | 2.56 | 1.67 | NT | NT |
| 62 | 2.65 | 1.17 | NT | NT |
| 63 | >25 | 0.555 | NT | NT |
| 64 | 0.051 | 0.017 | 5.05 | 7.03 |
| 65 | 0.007 | 0.008 | 0.699 | 0.547 |
| 66 | 0.94 | 0.3 | NT | NT |
| 67 | 0.512 | 0.199 | NT | NT |
| 68 | 0.004 | 0.003 | 0.732 | 0.658 |
| 69 | 0.004 | 0.002 | 1 | 0.514 |
| 70 | 0.003 | 0.007 | 0.012 | 0.049 |
| 71 | 0.038 | 0.05 | 4.79 | 3.36 |
| 72 | 0.023 | 0.018 | 2.68 | 2.6 |
| 73 | 0.015 | 0.017 | 0.422 | 0.715 |
| 74 | 0.007 | 0.007 | 0.148 | 0.386 |
| 75 | 0.005 | 0.003 | 0.139 | 0.297 |
| 76 | 0.01 | 0.008 | 0.191 | 0.674 |
| 77 | 0.003 | 0.002 | 0.038 | 0.062 |
| 78 | 0.226 | 0.090 | >10 | 6.03 |
| 79 | 0.026 | 0.026 | >10 | 3.43 |
| 80 | 0.010 | 0.004 | 0.649 | 0.607 |
| 81 | 0.003 | 0.004 | 0.03 | 0.099 |
| 82 | 0.002 | 0.002 | 0.042 | 0.138 |
| 83 | 0.003 | 0.002 | 0.028 | 0.077 |
| 84 | 0.004 | 0.004 | 0.005 | 0.022 |
| 85 | 0.004 | 0.001 | 0.064 | 0.273 |
| 86 | 0.004 | 0.002 | 0.054 | 0.149 |
| 87 | 0.007 | 0.010 | 0.006 | 0.016 |
| 88 | 0.004 | 0.004 | 0.003 | 0.009 |
| 89 | 0.004 | 0.004 | 0.080 | 0.137 |
| 90 | 0.004 | 0.003 | 0.454 | 0.79 |
| 91 | 0.019 | 0.013 | 4.47 | 1.78 |
| 92 | 0.024 | 0.033 | 0.880 | 0.829 |
| 93 | 0.005 | 0.006 | 0.006 | 0.009 |
| 94 | 0.005 | 0.004 | 0.110 | 0.152 |
| 95 | 0.010 | 0.010 | 0.199 | 0.23 |
| 96 | 0.007 | 0.005 | 0.095 | 0.213 |
| 97 | 0.006 | 0.006 | 0.069 | 0.178 |
| 98 | 0.013 | 0.013 | 0.587 | 1.05 |
| 99 | 0.003 | 0.003 | 0.005 | 0.019 |
| 100 | 0.008 | 0.006 | 0.945 | 0.663 |
| 101 | 0.07 | 0.035 | 2.92 | 1.71 |
| 102 | 0.144 | 0.055 | NT | 10.1 |
| 103 | 1.08 | 0.517 | NT | NT |
| 104 | 0.544 | 2.3 | NT | NT |
| 105 | 0.005 | 0.006 | 0.068 | 0.368 |
| 106 | 0.005 | 0.002 | 0.033 | 0.127 |
| 107 | 0.004 | 0.003 | 0.041 | 0.167 |
| 108 | 11.7 | 0.671 | NT | 0.42 |
| 109 | 0.004 | 0.003 | 0.017 | 0.029 |
| 110 | 0.004 | 0.003 | 0.01 | 0.026 |
| 111 | 0.002 | 0.001 | 0.028 | 0.051 |
| 112 | 0.002 | 0.002 | 0.037 | 0.09 |
| 113 | 0.003 | 0.002 | 0.102 | 0.453 |
| 114 | 0.016 | 0.004 | 0.985 | 1.42 |
| 115 | 0.032 | 0.023 | 2.74 | 2.79 |
| 116 | 0.03 | 0.005 | 0.254 | 0.454 |
| 117 | 0.013 | 0.007 | 0.411 | 0.899 |
| 118 | 0.14 | 0.039 | 6.78 | 4.95 |
| 119 | 0.005 | 0.004 | 0.156 | 0.276 |
| 120 | 0.008 | 0.004 | 1.16 | 1.17 |
| 121 | 0.026 | 0.008 | 5.53 | 2.85 |
| 122 | 0.005 | 0.005 | 0.167 | 0.282 |
| 123 | 0.012 | 0.012 | 1.99 | 2.24 |
| 124 | 0.034 | 0.019 | 4.32 | 2.41 |
| 125 | 0.114 | 0.039 | >10 | >10 |
| 126 | 0.006 | 0.002 | 0.388 | 1.22 |
| 127 | 0.012 | 0.003 | 3.3 | 7.5 |
| 128 | 0.003 | 0.004 | 0.308 | 0.243 |
| 129 | 0.004 | 0.005 | 0.029 | 0.083 |
| 130 | 0.003 | 0.005 | 0.052 | 0.102 |
| 131 | 0.009 | 0.005 | 0.682 | 0.926 |
| 132 | 0.016 | 0.006 | 1.69 | 1.2 |
| 133 | 0.007 | 0.006 | 2.43 | 2.04 |
| 134 | 0.004 | 0.007 | 0.057 | 0.074 |
| 135 | 0.01 | 0.009 | 2.12 | 2.27 |
| 136 | 0.056 | 0.021 | 5.35 | 4.16 |
| 137 | 0.037 | 0.034 | 0.411 | 1.71 |
| 138 | 0.003 | 0.004 | 0.072 | 0.216 |
| 139 | 0.005 | 0.004 | 0.008 | 0.044 |
| 140 | 0.002 | 0.001 | 0.011 | 0.07 |
| 141 | 0.002 | 0.001 | 0.01 | 0.093 |
| 142 | 0.002 | 0.002 | 0.006 | 0.019 |
| 143 | 0.003 | 0.003 | 0.013 | 0.046 |
| 144 | 0.003 | 0.003 | 0.023 | 0.041 |
| 145 | 0.002 | 0.003 | 0.011 | 0.039 |
| 146 | 0.001 | 0.003 | 0.083 | 0.129 |
| 147 | 0.003 | 0.002 | 0.003 | 0.025 |
| 148 | 0.002 | 0.002 | 0.083 | 0.143 |
| 149 | 0.005 | 0.002 | 0.939 | 0.68 |
| 150 | 0.003 | 0.003 | 0.03 | 0.094 |
| 151 | 0.004 | 0.003 | 0.065 | 0.123 |
| 152 | 0.005 | 0.004 | 0.073 | 0.076 |
| 153 | 0.006 | 0.003 | 0.349 | 0.419 |
| 154 | 0.013 | 0.012 | 0.879 | 1.03 |
| 155 | 0.005 | 0.003 | 0.639 | 0.667 |
| 156 | 0.007 | 0.003 | 1.18 | 0.881 |
| 157 | 0.004 | 0.004 | 0.009 | 0.046 |
| 158 | 0.007 | 0.004 | 0.228 | 0.511 |
| 159 | 0.003 | 0.005 | 0.013 | 0.077 |
| 160 | 0.005 | 0.006 | 0.41 | 0.754 |
| 161 | 0.075 | 0.079 | 10 | 9.75 |
| 162 | 0.026 | 0.015 | 2.12 | 2.38 |
| 163 | 0.042 | 0.016 | 3.17 | 3.15 |
| 164 | 0.007 | 0.006 | 0.862 | 0.725 |
| 165 | 0.027 | 0.011 | 4.43 | 3.28 |
| 166 | 0.094 | 0.082 | 4.2 | 1.53 |
| 168 | 0.004 | 0.008 | 0.078 | 0.243 |
| 169 | 0.005 | 0.005 | 0.009 | 0.072 |
| 170 | 0.003 | 0.003 | 0.086 | 0.126 |
| 171 | 0.006 | 0.003 | 0.042 | 0.169 |
| 172 | 0.085 | 0.036 | 5.43 | 2.92 |
| 173 | 0.002 | 0.003 | 0.153 | 0.366 |
| 174 | 0.004 | 0.005 | 0.057 | 0.064 |
| 175 | 0.006 | 0.004 | 0.448 | 1.56 |
| 176 | 0.011 | 0.007 | 0.545 | 2.65 |
| 178 | 0.003 | 0.005 | NT | 0.143 |
| 179 | 0.002 | 0.004 | NT | 0.046 |
| 180 | 0.003 | 0.004 | 0.003 | 0.018 |
| 181 | 0.001 | 0.001 | 0.003 | 0.018 |
| 182 | 0.001 | 0.002 | 0.004 | 0.033 |
| 183 | 0.002 | 0.004 | 1.48 | 5.4 |
| 184 | 0.001 | 0.001 | 0.002 | 0.011 |
| 185 | 0.001 | 0.001 | 0.001 | 0.016 |
| 186 | 0.003 | 0.003 | 0.001 | 0.005 |
| 187 | 0.002 | 0.001 | 0.005 | 0.016 |
| 188 | 0.002 | 0.002 | 0.002 | 0.016 |
| 189 | 0.004 | 0.003 | 0.001 | 0.002 |
| 190 | 0.004 | 0.005 | 0.001 | 0.004 |
| 191 | 0.003 | 0.004 | 0.004 | 0.038 |
| 192 | 0.004 | 0.002 | 0.15 | 0.532 |
| 193 | 0.015 | 0.01 | 0.482 | 1.08 |
| 194 | 0.014 | 0.01 | 0.843 | 0.815 |
| 195 | 0.04 | 0.017 | 2.38 | 2.67 |
| 196 | 0.037 | 0.02 | 1.36 | 2.31 |
| 197 | 0.108 | 0.054 | 4.03 | >10 |
| 198 | 0.042 | 0.018 | 5.27 | 2.93 |
| 199 | 0.003 | 0.004 | 0.009 | 0.07 |
| 200 | 0.004 | 0.004 | 0.011 | 0.054 |
| 201 | 0.003 | 0.006 | 0.016 | 0.045 |
| 202 | 0.004 | 0.004 | 0.009 | 0.175 |
| 203 | 0.006 | 0.002 | 0.062 | 0.171 |
| 204 | 0.002 | 0.003 | 0.026 | 0.098 |
| 205 | 0.006 | 0.007 | 0.071 | 0.293 |
| 206 | 0.03 | 0.042 | 0.116 | 0.705 |
| 207 | 0.007 | 0.007 | 0.202 | 0.348 |
| 208 | 0.018 | 0.008 | 0.484 | 0.843 |
| 209 | 0.068 | 0.024 | 1.62 | 2.57 |
| 210 | 0.044 | 0.069 | NT | NT |
| 211 | 0.018 | 0.029 | 6.29 | 8.75 |
| 212 | 0.002 | 0.003 | 0.514 | 0.784 |
| 213 | 0.011 | 0.006 | 1.46 | 2.3 |
| 214 | 0.013 | 0.005 | 1.7 | 1.21 |
| 216 | 0.005 | 0.009 | NT | 0.834 |
| 217 | 0.004 | 0.004 | 0.218 | 0.414 |
| 218 | 0.006 | 0.006 | 1.46 | 1.14 |
| 219 | 0.001 | 0.002 | 0.092 | 0.531 |
| 220 | 0.06 | 0.022 | 2.86 | 4.4 |
| 221 | 0.001 | 0.001 | 0.139 | 0.325 |
| 222 | 0.255 | 0.074 | >10 | >10 |
| 223 | 0.044 | 0.015 | 0.894 | 3.03 |
| 224 | 0.274 | 0.041 | 7.62 | >10 |
| 225 | 0.057 | 0.044 | 0.079 | 0.256 |
| 226 | 0.044 | 0.007 | >10 | >10 |
| 227 | 0.003 | 0.004 | 0.048 | 0.308 |
| 228 | 0.002 | 0.002 | 0.077 | 0.302 |
| 229 | 0.007 | 0.004 | 0.222 | 0.486 |
| 230 | 0.004 | 0.001 | 0.033 | 0.068 |
| 231 | 0.004 | 0.005 | 0.015 | 0.068 |
| 232 | 0.001 | 0.002 | 0.003 | 0.008 |
| 233 | 0.001 | 0.001 | 0.005 | 0.027 |
| 234 | 0.001 | 0.002 | NT | 0.022 |

NT: not tested.

### REFERENCES

All references, for example, a scientific publication or patent application publication, cited herein are incorporated herein by reference in their entirety and for all purposes to the same extent as if each reference was specifically and individually indicated to be incorporated by reference in its entirety for all purposes.

What is claimed is:

1. A compound of Formula (I): 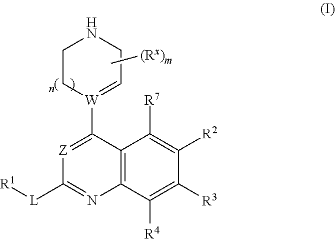
or a pharmaceutically acceptable salt of said compound, wherein; [symbol]
is a single bond or a double bond;
W is C, CH or N;
n is 0, 1, 2, or 3;
m is 0, 1, 2, 3 or 4;
each R^{x} is hydroxyl, halogen, oxo, cyano, C₁₋₄ alkyl, C₁₋₄ alkoxy, C₁₋₄ haloalkyl, —T—R^{y} or two R^{x} taken together can form a bridged ring, wherein the bridge is selected from one of the following: —C₁₋₄ alkylene, —O—C₁₋₄ alkylene, —C₁₋₄ alkylene-O—C₁₋₄ alkylene- or —C₁₋₄ alkylene-S—C₁₋₄ alkylene-;
Z is CH, CR′ or N;
R′ is halogen, cyano or C₁₋₄ alkyl;
L is a bond, C₁₋₆ alkylene, C₁₋₆ alkenylene, —O—C₁₋₆ alkylene, —S—C₁₋₆ alkylene, NR^{z}, O or S;
R¹ is hydroxyl, aryl, heteroaryl, C₃₋₅ cycloalkyl or heterocycloalkyl optionally substituted with 0-3 occurrences of R⁵;
R² is hydrogen, hydroxyl, halogen, amino, C₁₋₄ alkyl, C₁₋₄ alkoxy, C₁₋₄ haloalkyl, C₂₋₄ alkenyl, C₂₋₄ alkynyl, C₃₋₈ cycloalkyl or cyano;
R³ is aryl or heteroaryl optionally substituted with 0-4 occurrences of R⁶;
R⁴ is hydrogen, hydroxyl, halogen, C₁₋₄ alkyl, C₁₋₄ alkoxy, C₁₋₄ haloalkyl, C₂₋₄ alkenyl, C₂₋₄ alkynyl, C₃₋₈ cycloalkyl or cyano;
each R⁵ is halogen, hydroxyl, oxo, amino, C₁₋₄ alkyl or —T—R^{y};
each R⁶ is halogen, hydroxyl, amino, cyano, C₁₋₄ alkyl, C₂₋₄ alkenyl, C₂₋₄ alkynyl, C₁₋₄ haloalkyl or C₃₋₇ cycloalkyl;
R⁷ is hydrogen, halogen or C₁₋₄ alkyl;
T is C₁₋₄ alkylene, —O—, —S— or —C₁₋₄ alkylene-C(O)—;
R^{y} is halogen, hydroxyl, cyano or amino; and
R^{z} is hydrogen or C₁₋₄ alkyl;
wherein when W is N, [symbol] is a single bond, m is 2, 3, or 4 and two R^{x} are taken together to form a bridged ring, wherein the bridge is selected from one of the following: —C₁₋₄ alkylene, —C₁₋₄ alkylene-O—C₁₋₄ alkylene- or —C₁₋₄ alkylene-S—C₁₋₄ alkylene-.

2. The compound of claim 1, wherein R³ is not benzo[d]thiazole.

3. The compound of claim 1, wherein Z is CH or CR′.

4. The compound of claim 3, wherein R′ is fluorine, chlorine, methyl, ethyl or cyano.

5. The compound of claim 1, wherein Z is N.

6. The compound of claim 1, wherein W is N.

7. The compound of claim 1, wherein n is 1.

8. The compound of claim 1, wherein m is 2.

9. The compound of claim 8, wherein two R^{x} taken together form a bridged ring, wherein the bridge is selected from one of the following: —C₁₋₄ alkylene, —C₁₋₄ alkylene-O—, —C₁₋₄ alkylene-O—C₁₋₄ alkylene-, —C₁₋₄ alkylene-S—C₁₋₄ alkylene- or —C₁₋₄ alkylene-S—.

10. The compound of claim 9, wherein two R^{x} taken together form a bridged ring, wherein the bridge is selected from methylene, ethylene, propylene or -methylene-O-methylene-.

11. The compound of claim 1, wherein m is 3.

12. The compound of claim 11, wherein one R^{x} is halogen, C₁₋₄ alkyl, cyano, oxo or —T—R^{y} and the other two R^{x} are taken together to form a bridged ring, wherein the bridge is —C₁₋₄ alkylene or —O—C₁₋₄ alkylene or —C₁₋₄ alkylene-O—C₁₋₄ alkylene-.

13. The compound of claim 12, wherein one R^{x} is fluorine, methyl, ethyl, cyano, oxo, —CH₂OH or —CH₂CN and the other two R^{x} are taken together to form a bridged ring, wherein the bridge is methylene, ethylene, propylene or -methylene-O-methylene-.

14. The compound of claim 1, wherein m is 4.

15. The compound of claim 14, wherein two R^{x} are each independently C₁₋₄ alkyl or halogen and the other two R^{x} are taken together to form a bridged ring, wherein the bridge is C₁₋₄ alkylene or —C₁₋₄ alkylene-O—C₁₋₄ alkylene-.

16-19. (canceled)

20. The compound of claim 1, wherein

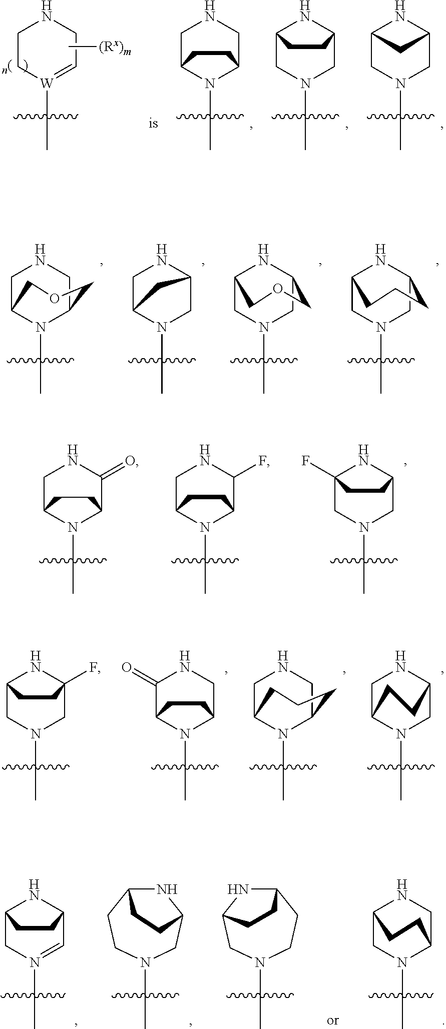

21. (canceled)

22. The compound of claim 1, wherein L is C₁₋₆ alkylene, —O—C₁₋₄ alkylene or C₁₋₆ alkenylene.

23. The compound of claim 22, wherein L is —O-methylene-, —O-ethylene-, —O-isopentanylene-, —O-n-propylene, —O—(2-methylpropylene)-, —O—(2-methylbutylene)-, —O—(2-ethylbutylene)-, —O—(1,2-dimethylpropylene)- or —O-(3-methylbutylene)-.

24. The compound of claim 1, wherein R¹ is hydroxyl, heterocycloalkyl or C₃₋₈ cycloalkyl, wherein each heterocycloalkyl or C₃₋₈ cycloalkyl is optionally substituted with 0-3 occurrences of R⁵.

25. The compound of claim 24, wherein R¹ is hydroxyl, 7-(hexahydro-1H-pyrrolizine), 2-pyrrolidine, 2-tetrahydrofuranyl, 2-imidazolyl, cyclopropyl, cyclobutyl or cyclopentyl.

26. The compound of claim 24, wherein each R⁵ is independently halogen, oxo, C₁₋₄ alkyl or hydroxyl.

27. The compound of claim 1, wherein —L—R¹ is

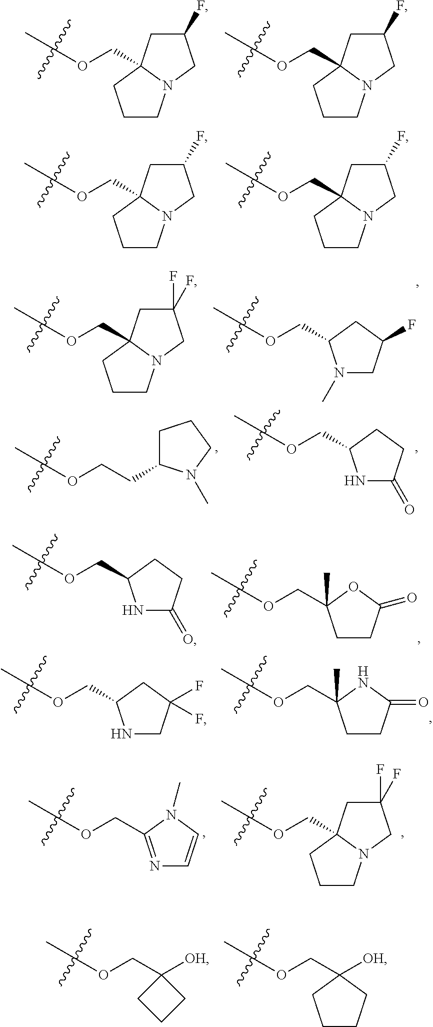

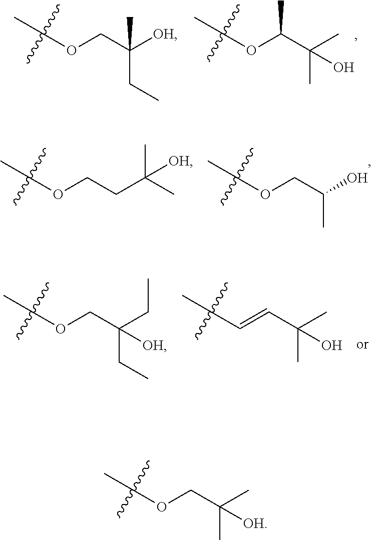

28. (canceled)

29. The compound of claim 1, wherein R³ is aryl or heteroaryl (e.g., phenyl or naphthyl) substituted with 0-3 occurrences of R⁶.

30. The compound of claim 29, wherein R³ is phenyl or naphthyl substituted with 0-3 occurrences of R⁶.

31. The compound of claim 29, wherein R³ is 8-quinolinyl, 5-quinolinyl, 4-isoquinolinyl, 1-isoquinolinyl, 8-isoquinolinyl, 4-(1H-indazolyl) or 7-(1H-indazolyl) substituted with 0-3 occurrences of R⁶.

32. The compound of claim 30 or 31, where each R⁶ is independently chlorine, fluorine, amino, cyano, methyl, ethyl, hydroxyl, ethenyl or ethynyl.

33. The compound of claim 1, wherein R³ is

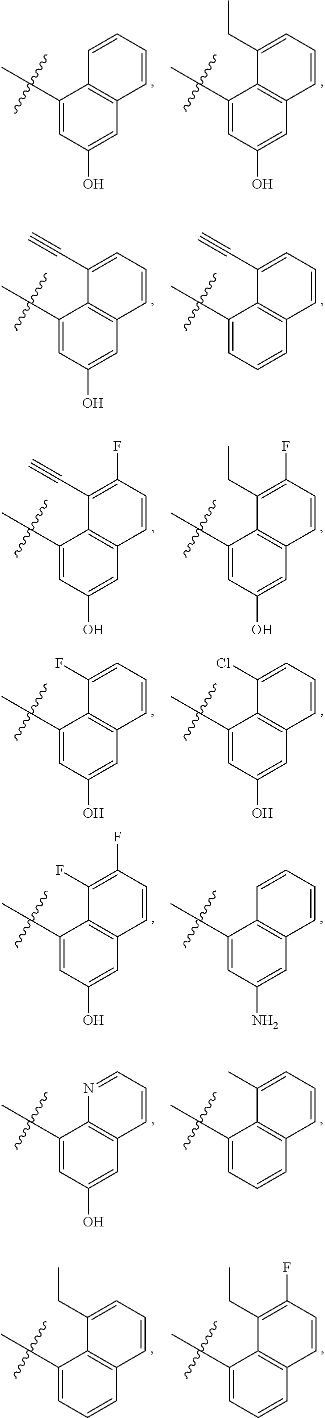

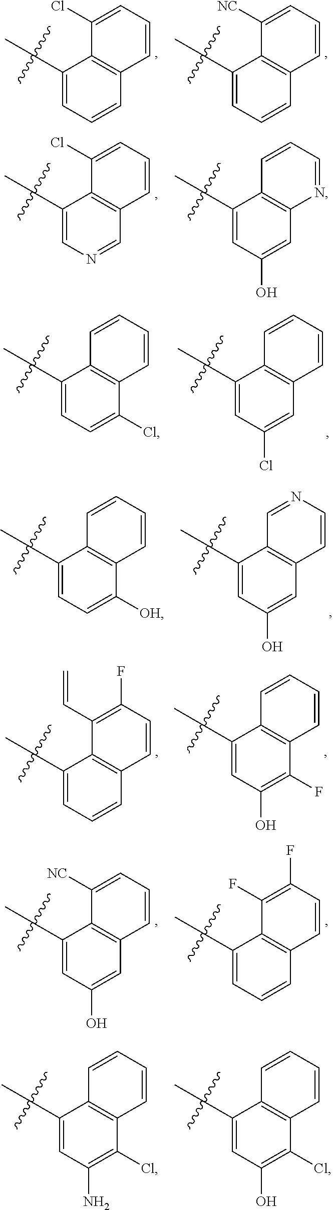

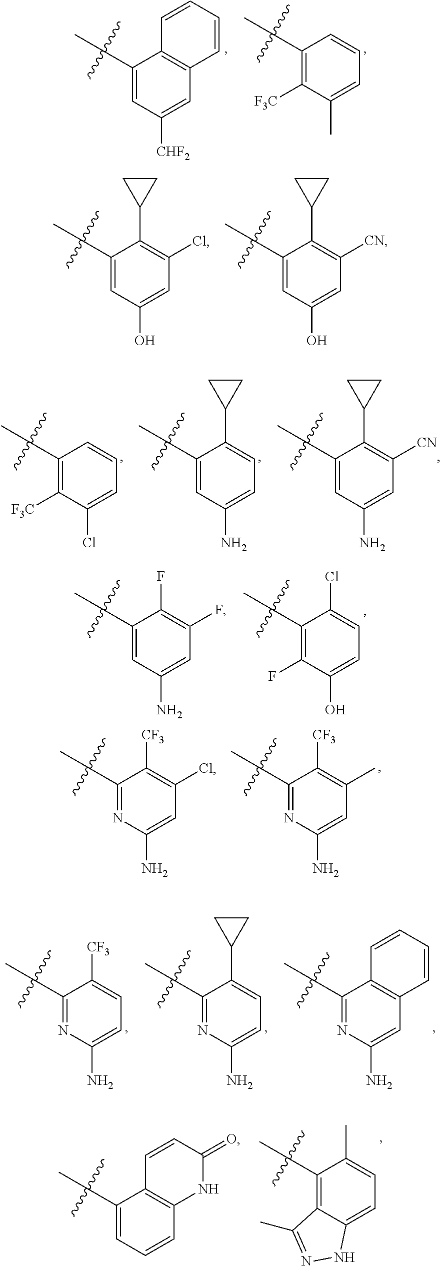

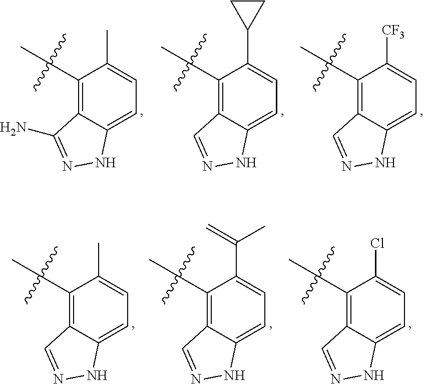 [SMILES: *c1c(C)ccc2[nH]nc(N)c12 ; *c1c(C2CC2)ccc2[nH]ncc12 ; *c1c(C(F)(F)F)ccc2[nH]ncc12 ; *c1c(C)ccc2[nH]ncc12 ; *c1c(C(=C)C)ccc2[nH]ncc12 ; *c1c(Cl)ccc2[nH]ncc12]

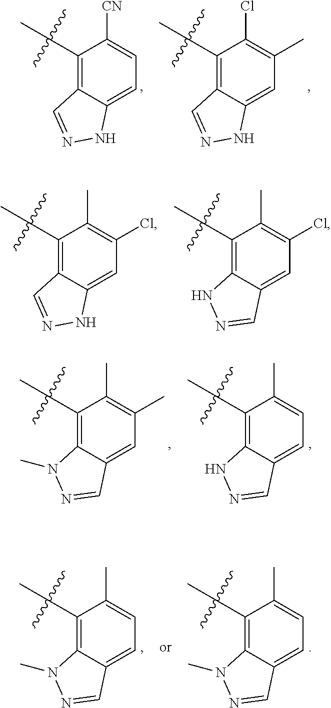

34. (canceled)

35. The compound of claim 1, wherein R² is hydrogen, fluorine, chlorine, cyano, amino, methyl, ethyl or ethenyl.

36. The compound of claim 1, wherein R⁴ is hydrogen, fluorine, methyl or hydroxyl.

37. The compound of claim 1, wherein R⁷ is hydrogen, methyl or fluorine.

38. The compound of claim 1, wherein the compound is selected from one of the following compounds:
4-(4-((1R,5S)-3,8-diazabicyclo[3.2.1]octan-3-yl)-6,8-difluoro-2-(((2R,7aS)-2-fluorotetrahydro-1H-pyrrolizin-7a(5H)-yl)methoxy)quinazolin-7-yl)-5-ethynyl-6-fluoronaphthalen-2-ol;
4-(4-((1R,5S)-3,8-diazabicyclo[3.2.1]octan-3-yl)-6,8-difluoro-2-(((2R,7aS)-2-fluorotetrahydro-1H-pyrrolizin-7a(5H)-yl)methoxy)quinazolin-7-yl)-5-ethynylnaphthalen-2-ol;
4-(4-((1R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)quinazolin-7-yl)-5-ethynyl-6-fluoronaphthalen-2-ol;
4-(4-((1R,5S)-3,8-diazabicyclo[3.2.1]octan-3-yl)-6,8-difluoro-2-(((2R,7aS)-2-fluorotetrahydro-1H-pyrrolizin-7a(5H)-yl)methoxy)quinazolin-7-yl)-5-ethyl-6-fluoronaphthalen-2-ol;
4-(4-((1S,4S)-2,5-diazabicyclo[2.2.2]octan-2-yl)-6,8-difluoro-2-(((2R,7aS)-2-fluorotetrahydro-1H-pyrrolizin-7a(5H)-yl)methoxy)quinazolin-7-yl)-5-ethyl-6-fluoronaphthalen-2-ol;
4-(4-((1S,4S)-2,5-diazabicyclo[2.2.2]octan-2-yl)-8-fluoro-2-(((2R,7aS)-2-fluorotetrahydro-1H-pyrrolizin-7a(5H)-yl)methoxy)quinazolin-7-yl)-5-ethynyl-6-fluoronaphthalen-2-ol;
4-(4-((1R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)quinazolin-7-yl)-5-ethyl-6-fluoronaphthalen-2-ol;
4-(4-((1R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)quinazolin-7-yl)-5-ethynylnaphthalen-2-ol;
4-(4-((1R,5S)-3,8-diazabicyclo[3.2.1]octan-8-yl)-8-fluoro-2-(((2R,7aS)-2-fluorotetrahydro-1H-pyrrolizin-7a(5H)-yl)methoxy)quinazolin-7-yl)-5-ethynyl-6-fluoronaphthalen-2-ol;
4-(4-((1S,4S)-2,5-diazabicyclo[2.2.2]octan-2-yl)-6,8-difluoro-2-(((2R,7aS)-2-fluorotetrahydro-1H-pyrrolizin-7a(5H)-yl)methoxy)quinazolin-7-yl)-5-ethynyl-6-fluoronaphthalen-2-ol;
4-(4-((1S,4S)-2,5-diazabicyclo[2.2.2]octan-2-yl)-6,8-difluoro-2-(((2R,7aS)-2-fluorotetrahydro-1H-pyrrolizin-7a(5H)-yl)methoxy)quinazolin-7-yl)-5-ethynylnaphthalen-2-ol;
4-(4-((1R,5S)-3,8-diazabicyclo[3.2.1]octan-3-yl)-6-fluoro-2-(((2R,7aS)-2-fluorotetrahydro-1H-pyrrolizin-7a(5H)-yl)methoxy)quinazolin-7-yl)-5-ethynyl-6-fluoronaphthalen-2-ol;
4-(4-((1R,5S)-3,8-diazabicyclo[3.2.1]octan-8-yl)-8-fluoro-2-(((2R,7aS)-2-fluorotetrahydro-1H-pyrrolizin-7a(5H)-yl)methoxy)quinazolin-7-yl)-5-ethynylnaphthalen-2-ol;
4-(4-((1R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)quinazolin-7-yl)-5,6-difluoronaphthalen-2-ol;
4-(4-((1R,5S)-3,8-diazabicyclo[3.2.1]octan-3-yl)-6,8-difluoro-2-(((2R,7aS)-2-fluorotetrahydro-1H-pyrrolizin-7a(5H)-yl)methoxy)quinazolin-7-yl)-5,6-difluoronaphthalen-2-ol;
4-(4-((1R,5S)-3,8-diazabicyclo[3.2.1]octan-3-yl)-6,8-difluoro-2-(((2R,7aS)-2-fluorotetrahydro-1H-pyrrolizin-7a(5H)-yl)methoxy)quinazolin-7-yl)-5-ethylnaphthalen-2-ol;
4-(4-((1S,4S)-2,5-diazabicyclo[2.2.2]octan-2-yl)-8-fluoro-2-(((2R,7aS)-2-fluorotetrahydro-1H-pyrrolizin-7a(5H)-yl)methoxy)quinazolin-7-yl)-5-ethylnaphthalen-2-ol;
4-(4-((1S,4S)-2,5-diazabicyclo[2.2.2]octan-2-yl)-6,8-difluoro-2-(((2R,7aS)-2-fluorotetrahydro-1H-pyrrolizin-7a(5H)-yl)methoxy)quinazolin-7-yl)-5,6-difluoronaphthalen-2-ol;
4-(4-((1S,4S)-2,5-diazabicyclo[2.2.2]octan-2-yl)-6,8-difluoro-2-(((2R,7aS)-2-fluorotetrahydro-1H-pyrrolizin-7a(5H)-yl)methoxy)quinazolin-7-yl)-5-ethylnaphthalen-2-ol;
4-(4-((1R,5S)-3,8-diazabicyclo[3.2.1]octan-3-yl)-6,8-difluoro-2-(((2R,7aS)-2-fluorotetrahydro-1H-pyrrolizin-7a(5H)-yl)methoxy)quinazolin-7-yl)naphthalen-2-ol;
4-(4-((1R,5S)-3,8-diazabicyclo[3.2.1]octan-3-yl)-6-fluoro-2-(((2R,7aS)-2-fluorotetrahydro-1H-pyrrolizin-7a(5H)-yl)methoxy)quinazolin-7-yl)-5-ethynylnaphthalen-2-ol;
4-(4-((1R,5S)-3,8-diazabicyclo[3.2.1]octan-3-yl)-2-(((2R,7aS)-2-fluorotetrahydro-1H-pyrrolizin-7a(5H)-yl)methoxy)-8-methylquinazolin-7-yl)-5-ethynylnaphthalen-2-ol;
4-(4-((1R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)quinazolin-7-yl)naphthalen-2-ol;
3-(4-((1R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)quinazolin-7-yl)-5-chloro-4-cyclopropylphenyl;
3-(4-((1R,5S)-3,8-diazabicyclo[3.2.1]octan-3-yl)-6,8-difluoro-2-(((2R,7aS)-2-fluorotetrahydro-1H-pyrrolizin-7a(5H)-yl)methoxy)quinazolin-7-yl)-5-chloro-4-cyclopropylphenyl;
4-(4-((1S,4S)-2,5-diazabicyclo[2.2.2]octan-2-yl)-8-fluoro-2-(((2R,7aS)-2-fluorotetrahydro-1H-pyrrolizin-7a(5H)-yl)methoxy)quinazolin-7-yl)-5-ethyl-6-fluoronaphthalen-2-ol;
4-((1R,5S)-3,8-diazabicyclo[3.2.1]octan-3-yl)-7-(8-ethyl-7-fluoro-3-hydroxynaphthalen-1-yl)-2-(((2R,7aS)-2-fluorotetrahydro-1H-pyrrolizin-7a(5H)-yl)methoxy)quinazolin-6-ol;
3-(4-((1S,4S)-2,5-diazabicyclo[2.2.2]octan-2-yl)-6,8-difluoro-2-(((2R,7aS)-2-fluorotetrahydro-1H-pyrrolizin-7a(5H)-yl)methoxy)quinazolin-7-yl)-5-chloro-4-cyclopropylphenyl;
4-(4-((1S,4S)-2,5-diazabicyclo[2.2.2]octan-2-yl)-6,8-difluoro-2-(((2R,7aS)-2-fluorotetrahydro-1H-pyrrolizin-7a(5H)-yl)methoxy)quinazolin-7-yl)-5,6-difluoronaphthalen-2-ol;
5-ethyl-6-fluoro-4-(8-fluoro-4-(1-fluoro-3,8-diazabicyclo[3.2.1]octan-3-yl)-2-(((2R,7aS)-2-fluorotetrahydro-1H-pyrrolizin-7a(5H)-yl)methoxy)quinazolin-7-yl)naphthalen-2-ol;
4-(4-((1R,5S)-3,8-diazabicyclo[3.2.1]octan-3-yl)-6,8-difluoro-2-(((2R,7aS)-2-fluorotetrahydro-1H-pyrrolizin-7a(5H)-yl)methoxy)quinazolin-7-yl)-5-chloronaphthalen-2-ol;
4-(4-((1R,5S)-3,8-diazabicyclo[3.2.1]octan-3-yl)-2-(((2R,7aS)-2-fluorotetrahydro-1H-pyrrolizin-7a(5H)-yl)methoxy)quinazolin-7-yl)-5-ethyl-6-fluoronaphthalen-2-ol;
4-((1R,5S)-3,8-diazabicyclo[3.2.1]octan-8-yl)-7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)quinazolin-6-ol;
4-(4-((1R,5S)-3,8-diazabicyclo[3.2.1]octan-3-yl)-2-(((2R,7aS)-2-fluorotetrahydro-1H-pyrrolizin-7a(5H)-yl)methoxy)quinazolin-7-yl)-5,6-difluoronaphthalen-2-ol;
4-(4-((1S,4S)-2,5-diazabicyclo[2.2.2]octan-2-yl)-6-fluoro-2-(((2R,7aS)-2-fluorotetrahydro-1H-pyrrolizin-7a(5H)-yl)methoxy)quinazolin-7-yl)-5-ethyl-6-fluoronaphthalen-2-ol;
4-(4-((1R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)-5-methylquinazolin-7-yl)-5-ethyl-6-fluoronaphthalen-2-ol;
4-(4-((1R,5S)-3,8-diazabicyclo[3.2.1]octan-3-yl)-5,8-difluoro-2-(((2R,7aS)-2-fluorotetrahydro-1H-pyrrolizin-7a(5H)-yl)methoxy)quinazolin-7-yl)-5-ethyl-6-fluoronaphthalen-2-ol;
4-(4-((1S,4S)-2,5-diazabicyclo[2.2.2]octan-2-yl)-8-fluoro-2-(((2R,7aS)-2-fluorotetrahydro-1H-pyrrolizin-7a(5H)-yl)methoxy)quinazolin-7-yl)-5,6-difluoronaphthalen-2-ol;
4-((1R,5S)-3,8-diazabicyclo[3.2.1]octan-3-yl)-7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)quinazolin-6-ol;
4-(4-((1R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)quinazolin-7-yl)-5-ethylnaphthalen-2-ol;
4-(4-((1R,5S)-3,8-diazabicyclo[3.2.1]octan-3-yl)-6-chloro-8-fluoro-2-(((2R,7aS)-2-fluorotetrahydro-1H-pyrrolizin-7a(5H)-yl)methoxy)quinazolin-7-yl)-5-ethyl-6-fluoronaphthalen-2-ol;
4-(4-((1R,5S)-3-oxa-7,9-diazabicyclo[3.3.1]nonan-9-yl)-8-fluoro-2-(((2R,7aS)-2-fluorotetrahydro-1H-pyrrolizin-7a(5H)-yl)methoxy)quinazolin-7-yl)-5-ethyl-6-fluoronaphthalen-2-ol;
4-(4-((1R,5S)-3,8-diazabicyclo[3.2.1]octan-3-yl)-6-fluoro-2-(((2R,7aS)-2-fluorotetrahydro-1H-pyrrolizin-7a(5H)-yl)methoxy)quinazolin-7-yl)-5-ethyl-6-fluoronaphthalen-2-ol;
4-(4-((1R,5S)-3,8-diazabicyclo[3.2.1]octan-3-yl)-6,8-difluoro-2-(((2R,7aS)-2-fluorotetrahydro-1H-pyrrolizin-7a(5H)-yl)methoxy)quinazolin-7-yl)-5-fluoronaphthalen-2-ol;
4-(4-((1S,4S)-2,5-diazabicyclo[2.2.2]octan-2-yl)-6-chloro-8-fluoro-2-(((2R,7aS)-2-fluorotetrahydro-1H-pyrrolizin-7a(5H)-yl)methoxy)quinazolin-7-yl)-5-ethyl-6-fluoronaphthalen-2-ol;
4-(4-((1R,5S)-3,8-diazabicyclo[3.2.1]octan-3-yl)-6,8-difluoro-2-(((2R,7aR)-2-fluorotetrahydro-1H-pyrrolizin-7a(5H)-yl)methoxy)quinazolin-7-yl)naphthalen-2-ol;
4-(4-((1S,4S)-2,5-diazabicyclo[2.2.2]octan-2-yl)-2-(((2R,7aS)-2-fluorotetrahydro-1H-pyrrolizin-7a(5H)-yl)methoxy)quinazolin-7-yl)-5-ethyl-6-fluoronaphthalen-2-ol;
4-(4-((1R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)quinazolin-7-yl)-5-fluoronaphthalen-2-ol;
4-(4-((1R,5S)-3,8-diazabicyclo[3.2.1]octan-8-yl)-6,8-difluoro-2-(((2R,7aS)-2-fluorotetrahydro-1H-pyrrolizin-7a(5H)-yl)methoxy)quinazolin-7-yl)-5-ethyl-6-fluoronaphthalen-2-ol; or
8-(4-((1R,5S)-3,8-diazabicyclo[3.2.1]octan-3-yl)-6,8-difluoro-2-(((2R,7aS)-2-fluorotetrahydro-1H-pyrrolizin-7a(5H)-yl)methoxy)quinazolin-7-yl)-6-hydroxy-1-naphthonitrile.

39. A pharmaceutical composition comprising the compound according to claim 1 or a pharmaceutically acceptable salt of said compound, and a pharmaceutically acceptable excipient.

40-46. (canceled)

47. A method of treating cancer in a subject in need thereof, the method comprising administering to the subject a therapeutically effective amount of the compound according to claim 1 or a pharmaceutically acceptable salt thereof or a pharmaceutical composition according to claim 39.

48-49. (canceled)

50. The method according to claim 47, wherein the cancer is non-small cell lung cancer, colorectal cancer, pancreatic cancer, appendiceal cancer, endometrial cancer, esophageal cancer, cancer of unknown primary, ampullary cancer, gastric cancer, small bowel cancer, sinonasal cancer, bile duct cancer, or melanoma.

51. The method according to claim 50, wherein the cancer is non-small cell lung cancer.

52. The method according to claim 50, wherein the cancer is colorectal cancer.

53. The method according to claim 50, wherein the cancer is pancreatic cancer.

54. (canceled)
